(19) United States
(12) Patent Application Publication
(10) Pub. No.: US 2024/0294613 A1
(43) Pub. Date: Sep. 5, 2024

(54) CROSS-NEUTRALIZING SARS-COV2 ANTIBODIES

(71) Applicants: The Broad Institute, Inc., Cambridge, MA (US); The General Hospital Corporation, Boston, MA (US); California Institute of Technology, Pasadena, CA (US)

(72) Inventors: Ramnik Xavier, Cambridge, MA (US); Johannes Scheid, Boston, MA (US); Christopher Barnes, Pasadena, CA (US); Pamela Bjorkman, Pasadena, CA (US); Basak Eraslan, Cambridge, MA (US)

(21) Appl. No.: 18/270,131

(22) PCT Filed: Dec. 30, 2021
(86) PCT No.: PCT/US21/65737

§ 371 Date: Jun. 28, 2023

Related U.S. Application Data
(60) Provisional application No. 63/132,695, filed on Dec. 31, 2020.
(60) Provisional application No. 63/155,220, filed on Mar. 1, 2021.

(51) Int. Cl. C07K 16/10; G01N 33/569

(52) U.S. Cl. CPC C07K 16/1003; G01N 33/56983; C07K 2317/21; C07K 2317/33; C07K 2317/34; C07K 2317/51; C07K 2317/55; C07K 2317/76; C07K 2317/92; G01N 2333/165; G01N 2469/20

(57) ABSTRACT

The present invention provides for cross-neutralizing SARS-COV2 antibodies. The antibodies can be used to treat SARS-CoV2 and variants thereof.

### CROSS-REFERENCE TO RELATED APPLICATIONS

This application claims the benefit of U.S. Provisional Application No. 63/132,695 filed Dec. 31, 2020 and 63/155,220 filed Mar. 1, 2021. The entire contents of the above-identified applications are hereby fully incorporated herein by reference.

### STATEMENT REGARDING FEDERALLY SPONSORED RESEARCH

This invention was made with government support under Grant Nos. DK43351, AI138938 and AI150464 awarded by the National Institutes of Health. The government has certain rights in the invention.

### REFERENCE TO AN ELECTRONIC SEQUENCE LISTING

The contents of the electronic sequence listing (BROD-5305WP_ST25.txt”; Size is 7,287,042 bytes and it was created on Dec. 29, 2021) is herein incorporated by reference in its entirety.

### TECHNICAL FIELD

The subject matter disclosed herein is generally directed to cross-neutralizing SARS-CoV2 antibodies.

### BACKGROUND

SARS-COV-2 is the third zoonotic betacoronavirus to cause a human outbreak after SARS-COV in 2002 and Middle East respiratory syndrome coronavirus (MERS-COV) in 2012 (de Wit et al., 2016). After the SARS-COV and MERS-COV outbreaks, limited numbers of neutralizing antibodies were isolated using phage display library techniques (Prabakaran et al., 2006; Sui et al., 2004) and Epstein-Barr virus transformation of B cells from recovered patients (Corti et al., 2015; Traggiai et al., 2004). The ability to characterize specific antibody responses in humans was advanced for pathogens such as human immunodeficiency virus 1 (HIV-1) (Scheid et al., 2009b; Wardemann et al., 2003), malaria (Triller et al., 2017), Zika virus (Robbiani et al., 2017), influenza (Wrammert et al., 2008) and viral hepatitis (Wang et al., 2020a) through the use of single-cell sorting followed by immunoglobulin gene sequencing. Since then, high-throughput single-cell RNA-seq (scRNA-seq) of virus-specific B cells has allowed simultaneous characterization of the clonal landscape of such cells and their associated transcriptional profiles (Neu et al., 2019). When combined with functional testing and structural characterization of selected monoclonal antibodies, this integrated approach should allow us to learn more about transcriptional pathways involved in the generation of efficient antiviral antibody responses and the roles of different B cell subpopulations (Horns et al., 2020; Mathew et al., 2020; Neu et al., 2019; Waickman et al., 2020).

Given the recent promising clinical results of therapeutic monoclonal antibodies against Ebola (Mulangu et al., 2019) and HIV-1 (Caskey et al., 2019; Scheid et al., 2016) numerous efforts have been undertaken to develop protective and potentially therapeutic antibodies against SARS-COV-2 (Baum et al., 2020; Brouwer et al., 2020; Cao et al., 2020b; Chen et al., 2020; Chi et al., 2020; Hurlburt et al., 2020; Ju et al., 2020; Liu et al., 2020; Lv et al., 2020; Pinto et al., 2020b; Robbiani et al., 2020; Rogers et al., 2020; Shi et al., 2020; Wan et al., 2020; Zost et al., 2020). These efforts were aided by structures that have revealed how the SARS-COV-2 spike binds to its angiotensin-converting enzyme 2 (ACE2) receptor (Yan et al., 2020), specificities of polyclonal antibody responses in COVID-19 convalescent individuals (Barnes et al., 2020b), and commonalities among receptor-binding domain (RBD)-binding monoclonal neutralizing antibodies that prevent infection (Barnes et al., 2020a; Tortorici, 2020; Yuan et al., 2020). Collectively, these structures provide foundations for potential therapeutic benefits by guiding choices of monoclonal antibody pairs for treatment cocktails, while informing structure-based engineering experiments to improve antibody potencies and/or create antibodies resistant to viral mutations. Furthermore, recent mapping of neutralizing SARS-COV-2 antibodies that target conserved spike epitopes (Lv et al., 2020; Piccoli et al., 2020) has the potential to guide structure-based immunogen design to elicit cross-reactive antibodies against zoonotic coronaviruses with spillover potential.

Citation or identification of any document in this application is not an admission that such a document is available as prior art to the present invention.

### SUMMARY

In one aspect, the present invention provides for an antibody or antigen binding fragment thereof that binds to the SARS-COV-2 receptor-binding domain (RBD) and/or SARS-CoV-2 spike trimer comprising: a complementarity-determining region 3 (CDR3) heavy chain and light chain pair that shares at least 90% identity with the heavy chain and/or light chain of a pair selected from the group consisting of: BG1-1, BG1-3 to BG1-28, BG4-1 to BG4-27, BG7-1 to BG7-7, BG7-9 to BG7-20, and BG10-1 to BG10-19 (SEQ ID NOS: 1-184); or a heavy chain and light chain pair that shares at least 90% identity with the heavy chain and/or light chain of a pair selected from the group consisting of: BG1-1, BG1-3 to BG1-28, BG4-1 to BG4-27, BG7-1 to BG7-7, BG7-9 to BG7-20, and BG10-1 to BG10-19 (SEQ ID NOS: 185-368). In certain embodiments, said antibody or antigen binding fragment is an IgG antibody or antigen binding fragment. In certain embodiments, said antibody or antigen binding fragment is a monoclonal antibody.

In certain embodiments, said CDR3 heavy chain and light chain pair or heavy chain and light chain pair is selected from the group consisting of: BG1-6, BG1-12, BG1-14, BG1-17, BG1-22, BG1-23, BG1-24, BG1-25, BG1-26, BG1-28, BG4-10, BG4-11, BG4-14, BG4-16, BG4-17, BG4-24, BG4-25, BG4-26, BG7-14, BG7-15, BG7-16, BG7-18, BG7-19, BG7-20, BG10-10, BG1014, and BG10-19. In certain embodiments, said CDR3 heavy chain and light chain pair or heavy chain and light chain pair is selected from the group consisting of: BG1-22, BG1-24, BG4-25, BG7-15, BG7-20, and BG10-19.

In certain embodiments, said heavy chain and light chain pair is BG7-15. In certain embodiments, said antibody or antigen binding fragment thereof binds the two “down”/one “up” RBD conformation on the SARS COV2 spike trimer with no glycan or interprotomer contacts. In certain embodiments, said antibody or antigen binding fragment recognizes an epitope in proximity to RBD residues 439-450. In certain embodiments, said antibody or antigen binding fragment blocks RBD-ACE2 interactions.

In certain embodiments, said heavy chain and light chain pair is BG7-20. In certain embodiments, said antibody or antigen binding fragment thereof binds a 2d/1u or 1d/2u binding state in the SARS COV2 spike trimer.

In certain embodiments, said heavy chain and light chain pair is BG10-19. In certain embodiments, said antibody or antigen binding fragment thereof binds to an all down RBD conformation in the SARS COV2 spike trimer. In certain embodiments, said antibody or antigen binding fragment recognizes an epitope in proximity to the N343-glycan. In certain embodiments, said antibody or antigen binding fragment light chain makes secondary contacts with an adjacent RBD. In certain embodiments, said antibody or antigen binding fragment heavy chain makes contacts with an epitope in proximity to the N343-glycan and the light chain contacts adjacent down RBDs and interacts with residues that overlap with the ACE2 receptor binding motif.

In certain embodiments, said heavy chain and light chain pair is BG1-24. In certain embodiments, said antibody or antigen binding fragment thereof binds both the up/down RBD conformation on the SARS COV2 spike trimer.

In certain embodiments, said heavy chain and light chain pair is BG1-22. In certain embodiments, said antibody or antigen binding fragment thereof binds only the up RBD conformation on the SARS COV2 spike trimer.

In certain embodiments, said antibody or antigen binding fragment is a VH3-53/3-66 antibody. In certain embodiments, said antibody or antigen binding fragment comprises a CDR3 having a consensus sequence according to FIG. 29. In certain embodiments, said heavy chain and light chain pair is BG4-25.

In another aspect, the present invention provides for an antibody or antigen binding fragment thereof that binds to the SARS, MERS and CoV2 spike trimers outside of the receptor-binding domain (RBD) comprising a complementarity-determining region 3 (CDR3) heavy chain and light chain pair that shares at least 90% identity with the heavy chain and/or light chain of a pair selected from the group consisting of LKA-1-17 (Table 7).

In certain embodiments, the antibody or antigen binding fragment is modified to enhance stabilization, in vivo half-life, neutralizing activity and/or dimerization. In certain embodiments, the antibody or antigen binding fragment is a fusion protein. In certain embodiments, the antibody or antigen binding fragment is fused to another antibody or antibody fragment, Fc domain, antigen binding domain, glutathione S-transferase (GST), and/or serum albumin. In certain embodiments, the antibody or antigen binding fragment comprises amino acid substitutions that increase antibody binding and/or viral neutralization activity.

In another aspect, the present invention provides for a method of treating a coronavirus infection comprising administering to a subject in need thereof one or more antibodies or antigen binding fragments of any embodiment herein. In certain embodiments, the coronavirus is selected from the group consisting of SARS-COV-2, SARS and MERS. In certain embodiments, the SARS-CoV-2 is a SARS-COV-2 variant. In certain embodiments, the SARS-COV-2 variant is selected from the group consisting of B.1.1.7 and B.1.351. In certain embodiments, BG10-19 is administered. In certain embodiments, BG10-19 and BG4-25 are administered.

In another aspect, the present invention provides for a method of identifying neutralizing antibodies from a subject infected with a virus comprising selecting antibodies expressed in B cells further expressing a transcriptional program, wherein the transcriptional program comprises one or more genes selected from the group consisting of: GRAMD1C, TMEM156, PDE4D, NFKBIA, S100A10, TKT, CAPG, CXCR3, CFLAR, HMGA1, MARCKSL1, PIM3, RHOF, MIF, ZFP36L1, NME2, MGAT4A, COCH, HOPX, ITGB2-AS1, BASP1, CD80, PAPSS1, CD70, LYPLAL1, LMNA, and FAS; or TKT, ARHGDIB, CFL1, CNN2, S100A10, HSPA8, HMGA1, PPIA, RAC2, CLIC1, SLC25A5, ARPC1B, SELL, PPP1CA, CAPZB, PPP1R18, CAPG, LDHB, S100A4, VIM, LTB, ANXA2, LCP1, TUBB, ACTG1, GAPDH, ACTB, PFN1, CORO1A, and TMSB10; or CD27, CD80, CD46 and CD86.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIGS. 1A-1E—SARS-COV-2 binding B cell populations in 14 recovered subjects; Representative FACS plots with the gating strategy for sorting of SARS-COV-2 S (FIG. 1A) and RBD binding (FIG. 1B) B cells. Pre-enriched CD20 cells were gated on live cells based on their appearance in side scatter (SSC) and forward scatter (FSC). Of these, CD19-FITC and SARS-CoV-2 S-APC or SARS-COV-2 RBD-APC double-positive cells were selected for sorting and sequencing. (FIG. 1C) Bar diagram showing the number of cells for which transcriptional profile and matching VDJ sequences were obtained in each subject against SARS-COV-2 S (turquoise) and SARS-COV-2 RBD (yellow). (FIG. 1D) Pie charts showing the total number of B cells sorted from each subject in the center of each pie and expanded B cell clones represented by differently colored pie slices. The white areas in each pie chart represent singlets. (FIG. 1E) Bar diagram showing the fraction (in %) of VH3-53 and VH3-66 genes combined among SARS-COV-2 S trimer (left), RBD binding cells (center) and all cells combined (right) in each donor compared to historic control memory B cells (Rubelt et al., 2012). Red stars indicate fractions with statistically significant difference (P value<0.005) to historic control memory B cells (Rubelt et al., 2012).

FIGS. 2A-2G—Antibody neutralization, cross-binding and cross-neutralization. (FIG. 2A) Dot plots showing ELISA binding of all expressed antibodies against SARS-COV-2 S (y-axis) and RBD (x-axis) (Table 3) expressed as area under the curve (AUC) for subjects BG1, BG4, BG7 and BG10, respectively. Red dots represent neutralizers and black dots non-neutralizers in a SARS-COV-2 pseudovirus assay (Tables 2 and 3). Green dots represent negative control antibody mGO53 (Wardemann et al., 2003). Two tailed P values were generated by calculating Spearman correlation coefficient. As in Table 3 high binding was defined as AUC>1, intermediate binding as an AUC 0.25-1 and low binding as an AUC<0.25. (FIG. 2B) Bar diagram showing the IC₅₀ values in μg/ml (y-axis) against SARS-COV-2 pseudovirus in red and authentic SARS-COV-2 virus in black for all antibodies that showed neutralizing activity against SARS-COV-2 pseudovirus (Table 2). Pearson correlation coefficient (r) and P value were calculated comparing results from both assays. (FIG. 2C) Bar diagram showing the ELISA AUC for all antibodies that showed neutralizing activity in a SARS-COV-2 pseudovirus assay (Table 2) against SARS-COV-2 S (black), SARS-COV-2 RBD (red), SARS-COV S (orange), SARS-COV RBD (magenta), MERS-COV S (dark grey) and MERS-COV RBD (light grey). The two antibodies showing cross reactivity, BG1-28 and BG10-19, are indicated with red stars. (FIG. 2D) In vitro pseudovirus neutralization assay comparing the potencies of antibodies BG1-28, BG10-19 and S309 against SARS-COV (Pinto et al., 2020a). Data points represent the average of duplicates, with error bars representing the standard deviation. (FIG. 2E) Normalized relative luminescence values in cell lysates after infection with SARS-COV-2 pseudoviruses harboring WT-D614G (black), B.1.1.7 (hot pink), or B.1.351 (green) sequences in the presence of indicated antibodies. (FIG. 2F) Bar diagram showing the ELISA AUC for all antibodies that showed neutralizing activity in a SARS-CoV-2 pseudovirus assay (Table 2) against SARS-COV-2 S (red), SARS-COV-2 RBD (brown), SARS-COV S (dark blue), SARS-COV RBD (light blue), MERS-COV S (dark grey) and MERS-CoV RBD (light grey). The three antibodies showing strong cross reactivity, BG1-28, BG10-14 and BG10-19, are indicated with red stars. (FIG. 2G) In vitro pseudovirus neutralization assay comparing the potencies of antibodies BG1-28, BG10-19 and S309 (left panel) or several SARS-CoV reactive antibodies and CR3022 (right panel) against SARS-COV. Data points for neutralization assays represent the average of duplicates, with error bars representing the standard deviation.

FIGS. 3A-3H—Cryo-EM structure of the BG10-19-S complex illustrates a distinct binding mode. (FIG. 3A) 3.3 Å cryo-EM density for the BG10-19-S trimer complex revealing three BG10-19 Fabs bound to a closed spike conformation. Side view (left panel) illustrates orientation with respect to the viral membrane (dashed line). Red circle (right panel) highlights quaternary epitope of BG10-19. (FIG. 3B) Close-up view of quaternary epitope highlighted in panel A (red circle) of BG10-19 (blue) involving bridging interactions that span neighboring protomers. (FIG. 3C) BG10-19 CDR loops (cartoon) mapped on the RBD surface. (FIG. 3D) Surface representation of BG10-19 quaternary epitope colored for BG10-19 heavy chain (steel blue) and light chain (light steel blue) interactions. (FIG. 3E) BG10-19 CDRH3 (sticks) buries into a concave pocket formed by core RBD residues near the N343-glycan (teal spheres). (FIG. 3F) BG10-19 CDRH2 (steel blue) interactions with RBD residue (light gray) R346. Potential cation-pi and hydrogen bond interactions are illustrated by dashed black lines. (FIG. 3G) Cryo-EM density map contoured at 5σ around the N343_{RBD}-glycan (teal sticks), which interfaces with the BG10-19 CDRH3 (steel blue) and CDRL2 (light steel blue) loops. Inset shows schematic of N343_{RBD}-glycan (Blue square: N-acetylglucosamine (GlcNAc), Green circle: Mannose (Man), Red triangle: Fucose (Fuc)). (FIG. 3H) SPR experiment to assess whether BG10-19 Fab blocks ACE2 binding on S trimer through inducing the closed prefusion state. SARS-COV-2 S trimer or RBD (black curves, Fab-S, or Fab-RBD complexes (colored curves) were injected over immobilized ACE2-CH3 (Tada et al., 2020).

FIGS. 4A-4E—B cell transcriptome analysis. (FIG. 4A) Heatmap displaying the relative expression levels of the top 15 marker genes of the 6 clusters (rows) across cells (columns) (Z-score normalization, row normalized). Marker genes that are known to be differentially expressed in particular B cell developmental stages are indicated (Table 4 for a full list of marker genes). (FIG. 4B) Uniform Manifold Approximation and Projection (UMAP) plot displaying the cells defined by their single-cell transcriptome measurements. The cells are colored based on their cluster assignments by the Louvain clustering algorithm. (FIG. 4C) Same UMAP plot as in (B) but with cells colored based on their heavy chain isotypes: IGHD in blue, IGHM in yellow, IGHA in red and IGHG in green. (FIG. 4D) Same UMAP plot as in (B) but cells colored based on antibody binding and neutralization: cells with non-tested antibodies in grey, highly potent SARS-COV-2 neutralizers (IC50<0.1 mg/ml in pseudovirus neutralization assay as either IgG1 or IgA monomer) in red, low neutralizers (IC_{50≥0.1} mg/ml) in yellow, non-neutralizing high binders in green and non-neutralizing low binders in blue (FIG. 9L, Tables 2 and 3). (FIG. 4E) Same UMAP plot as in (B) but cells with antibody CDRH3 shorter than 14 amino acids based on the IMGT definition (Lefranc et al., 2015) and VH3-53 or VH3-66 are shown in red and blue respectively. All other sorted and sequenced cells are shown in grey.

FIGS. 5A-5E—Cryo-EM structure of BG1-22-S provides insight into the VH3-53/VH3-66/long CDRH3 antibody binding mode. (FIG. 5A) Cryo-EM density for the BG1-22-S trimer complex. (FIG. 5B) Left panel: Close-up view of BG1-22 (orange) recognition of RBD epitope (gray surface). Right panels: Overlay of VH3-53 antibodies BG4-25 (green, this study), COVA2-39 (purple, PDB 7JMP), and C144 (blue, PDB 7K90) with BG1-22 (orange). (FIG. 5C) BG1-22 (orange) and BG4-25 (green) CDR loops mapped on the RBD (gray surface). Disordered residues in the BG1-22 CDRH3 are denoted by a black dashed line. (FIG. 5D) Flattened surface representation of BG1-22 (orange) and BG4-25 (green) with CDRH3 loops highlighted. The CDRH3 of BG1-22 is displaced from the canonical binding pocket along the RBD surface adopted by C102-like antibodies. (FIG. 5E) Surface representation of the BG1-22 RBD epitope (orange). Underlying residues are shown as sticks.

FIGS. 6A-6G—Cryo-EM structures of BG7-15, BG7-20, and BG1-24-S trimer complexes. (FIG. 6A) Cryo-EM density for the BG7-15-S trimer complex. (FIG. 6B) BG7-15 (HC: dark red, LC: salmon) targeting of its RBD (gray surface) epitope. Glycans are depicted as teal spheres. Color coding will be the same throughout the remaining panels. (FIG. 6C) BG7-15 CDR loops mapped on the RBD. (FIG. 6D) Surface representation of BG7-15 epitope. (FIG. 6E) Overlay of BG7-15 (red), REGN-10987 (gold, PDB 6XDG), and hACE2 (green, PDB 6M0J) on the RBD (gray surface). Despite non-overlapping epitopes, BG7-15 and REGN-10987 binding would sterically-hinder hACE2 interactions with the RBD. (FIG. 6F) Top down view of BG7-20 (HC: magenta, LC; pink) bound to S trimer. Inset: cryo-EM density of BG7-20-S trimer complex. (FIG. 6G) Close-up view of BG1-24 (HC: burnt orange, LC: wheat) bound to RBD. Interactions with the adjacent up RBD and N165_{NTD}-glycan are shown. Inset: cryo-EM density of BG1-24-S trimer complex.

FIGS. 7A-7R-Effects of RBD substitutions on monoclonal antibody binding and neutralization. (FIG. 7A) Heat map showing the log fold change in ELISA AUC of antibody Fabs binding to indicated SARS-COV-2 RBD mutants relative to wild-type SARS-COV-2 RBD binding. Mutations that lead to non-binding or decreases >20-fold relative to wild-type are shown as dark red. (FIG. 7B) Surface representation of the SARS-COV-2 RBD with monoclonal antibody footprints (BG1-22, orange; BG7-15, red; BG1-24, magenta; and BG10-19, blue) shown as dashed lines. Sites of individual RBD substitutions are shown as red spheres. (FIGS. 7C-7H) Normalized relative luminescence values in cell lysates after infection with SARS-COV-2 pseudoviruses harboring wild-type or RBD substitutions in the presence of indicated 6 antibodies. (FIG. 71) Summary table of IC₅₀ and IC₉₀ values measured for 6 different antibodies against pseudoviruses harboring wild-type or mutant RBDs. (FIG. 7J) Heat map showing the log fold change in ELISA AUC of antibody Fabs binding to indicated SARS-COV-2 RBD mutants relative to wild-type SARS-COV-2 RBD binding. Mutations that lead to non-binding or decreases >20-fold relative to wild-type are shown as dark red. (FIG. 7K-7P) Normalized relative luminescence values in cell lysates after infection with SARS-COV-2 pseudoviruses harboring wild-type or RBD substitutions in the presence of indicated 6 antibodies. (FIG. 7Q) Summary table of IC₅₀, IC₈₀ and IC50_{eff} values measured for 6 different antibodies and antibody combinations against SARS-COV-2 pseudoviruses. IC50eff values were calculated based on synergistic models previously described (West et al., 2010). (FIG. 7R) Infection of 293T/ACE2cl.22 cells by rVSV/SARS-COV-2/GFP encoding the indicated spike mutations in the presence of increasing amounts of BG10-19. Infection was quantified by FACS after 16 h, with mean and SD from two technical replicates shown.

FIGS. 8A-8P-Serum Neutralization against SARS-COV-2 pseudovirus and B cell repertoire characteristics (related to FIG. 1). (FIG. 8A) Serum ID₅₀ titers of all 14 study subjects against SARS-COV-2 pseudovirus. The blue dots represent subjects that were selected for monoclonal antibody isolation. (FIG. 8B) Correlation between ID₅₀ titers of all 14 study subjects against SARS-COV-2 pseudovirus and days between COVID-19 diagnosis and blood draw. P value was calculated based on the Pearson correlation coefficient (R). (FIG. 8C) Correlation between ID₅₀ titers of all 14 study subjects against SARS-COV-2 pseudovirus and age of the subjects in years. P value was calculated based on the Pearson correlation coefficient (R). (FIG. 8D) Serum ID₅₀ titers of all 14 study subjects against SARS-COV-2 pseudovirus grouped based on subject gender. P value was calculated using an unpaired two-tailed t-test. (FIG. 8E) Pie charts show the total number of B cells sorted from each subject in the center of each pie and differentially expanded pie slices represent the fraction of IgM/IgD (black), IgG (white) and IgA (grey) cells among all sorted cells. (FIG. 8F) Box plots showing absolute numbers of inferred nucleotide mutations per heavy chain V segment for all sorted cells (red bar), S-binding B cells (blue bar), RBD-binding B cells (green bar) and historic control memory B cells (brown bar) (Rubelt et al., 2012). The box limits are at the lower and upper quartiles and the center line indicates the median. (FIG. 8G) Box plots showing absolute numbers of inferred nucleotide mutations per heavy chain V segment for all sorted cells grouped by immunoglobulin isotypes IgD (pink), IgM (purple), IgA (green) and IgG (blue). The box limits are at the lower and upper quartiles, the center line indicates the median, the whiskers indicate 1.5× interquartile range and the dots represent outliers. P values were calculated using two tailed t-test and red brackets indicate statistical significance of the difference between samples with *P<=0.05, **P<=0.01, ***P<=0.001, ****P<=0.0001 and n/s indicating no statistical significance. P values were calculated using a two-tailed t-test. (FIG. 8H) Bar diagram showing the frequency of kappa and lambda light chains among all sorted cells (red), S-binding B cells (blue), RBD-binding B cells (green), historic control memory B cells (brown) (Rubelt et al., 2012). (FIG. 8I) Box plots showing the CDRH3 length in amino acids for all sorted cells (red), S-binding B cells (blue), RBD-binding B cells (green), historic control memory B cells (brown) (Rubelt et al., 2012). (FIG. 8J) Box plots showing the CDRH3 amino acid charges for all sorted cells (red), S-binding B cells (blue), RBD-binding B cells (green), historic control memory B cells (brown) (Rubelt et al., 2012). (FIG. 8K) Box plots showing the CDRH3 amino acid hydrophobicity scores for all sorted cells (red), S-binding B cells (blue), RBD-binding B cells (green), historic control memory B cells (brown) (Rubelt et al., 2012). (FIG. 8L) Box plots showing the CDRH3 length in amino acids for historic control memory B cells (brown) (Rubelt et al., 2012), IgD⁺ SARS COV2-binding B cells (pink), IgM⁺ SARS COV2-binding B cells (purple), IgA⁺ SARS CoV2-binding B cells (green), IgG⁺ SARS COV2-binding B cells (blue). (FIG. 8M) Box plots showing the CDRH3 amino acid charges for historic control memory B cells (brown) (Rubelt et al., 2012), IgD⁺ SARS COV2-binding B cells (pink), IgM⁺ SARS COV2-binding B cells (purple), IgA⁺ SARS COV2-binding B cells (green), IgG⁺ SARS COV2-binding B cells (blue). CDRH3 amino acid charges were calculated as in (FIG. 8J). (FIG. 8N) Box plots showing the CDRH3 amino acid hydrophobicity scores for historic control memory B cells (brown) (Rubelt et al., 2012), IgD⁺ SARS COV2-binding B cells (pink), IgM⁺ SARS COV2-binding B cells (purple), IgA⁺ SARS CoV2-binding B cells (green), IgG⁺ SARS COV2-binding B cells (blue). Hydrophobicity scores were calculated as in (FIG. 8K). For all box plots the box limits are at the lower and upper quartiles, the center line indicates the median, the whiskers indicate 1.5× interquartile range and the dots represent outliers. P values were calculated using two tailed t-test and red brackets indicate statistical significance of the difference between samples with stars indicating P values as in (FIG. 8G). (FIG. 8O) Distribution of clone sizes for each donor stratified by the clone isotype. Clones that contain cells from multiple isotypes are included as a data point in each of the respective groups. Numbers at the top of the violin plots display the percentage of cells from each isotype. Red brackets indicate statistical significance of the difference between isotypes with *P<=0.05, **P<=0.01, ***P<=0.001, ****P<=0.0001 and n/s indicating no statistical significance. P values were calculated using a two-tailed t-test. (FIG. 8P) Scatterplot displaying the relationship between clone size (number of cells, x-axis) and number of inferred mutations in the heavy chain V segment (y-axis). Each dot represents a cell or a group of cells within a clone with the same heavy chain isotype and number of inferred heavy chain mutations. Clonally related cells with different number of inferred heavy chain mutations are shown with the same color and grouped with violin plots. Pearson correlation coefficients and the p-values of each sub-plot are shown on separately.

FIGS. 9A-9M—Selection and testing of representative antibodies (related to FIG. 2). Venn diagrams summarizing all expanded clones, their immunoglobulin isotypes and selection of representative antibodies for donors BG1 (FIG. 9A), BG4 (FIG. 9B), BG7 (FIG. 9C) and BG10 (FIG. 9D). Colored circles show expanded B cell clones with the diameter of the circle corresponding to the size of the clone except for the largest clone in BG10 with 36 members. The numbers in the circles indicate the number of clonal members for all clones with more than 2 members. Red circles show expanded clones of which one representative antibody was produced and tested and blue circles show expanded clones of which no representative antibodies were tested. Red triangles represent singlets that were produced and tested. (FIG. 9E) Heatmap summarizing the polyreactivity ELISA results for all antibodies produced as IgG1 isolated from BG1, BG4, BG7 and BG10 against double strand DNA (dsDNA), single strand DNA (ssDNA), lipopolysaccharide (LPS), insulin (Ins) and streptavidin-APC (SA-APC) (Methods). OD⁴¹⁵ values less than 0.5 at an antibody concentration of 1 μg/ml are considered non-reactive and are indicated in white, OD⁴¹⁵ values between 0.5 and 1.9 at an antibody concentration of 1 μg/ml are considered moderately reactive and are indicated in orange, and OD⁴¹⁵ values of 2 and above at an antibody concentration of 1 μg/ml are considered strongly reactive and are indicated in red (Tiller et al., 2007). Results for highly-, moderately- and non-polyreactive control antibodies ED38, JB40 and mGO53 are included as indicated (Wardemann et al., 2003). (FIG. 9F) Dot plots summarizing IC₈₀ values from SARS-COV-2 pseudovirus neutralization assay in μg/ml (y-axis) for all antibodies isolated from subjects BG1, BG4, BG7 and BG10 and produced as IgG1 (Table 2). Pink dots represent antibodies isolated from IgG⁺ B cells, bright blue dots represent antibodies isolated from IgA⁺ B cells and black dots show antibodies isolated from IgM⁺ B cells. (FIG. 9G) Bar diagram showing the percent of neutralizing antibodies in a SARS-COV-2 pseudovirus neutralization assay among antibodies derived from IgM/IgD+ B cells (black), IgG⁺ B cells (white) and IgA⁺ B cells (grey) (Table 2). (FIG. 9H) Bar diagram showing the IC₈₀ values in μg/ml (y-axis) against SARS-CoV-2 pseudovirus in red and authentic SARS-COV-2 virus in black for all antibodies that showed neutralizing activity against SARS-COV-2 pseudovirus (Table 2). Pearson correlation coefficient (R) and P value were calculated comparing results from both assays. (FIG. 9I) Table summarizing neutralizing activity of antibodies BG10-19, BG1-22, BG4-25 and BG7-15 against SARS-COV-2 D614G, B.1.1.7 and B.1.351. Shown are IC50 and IC80 values in mg/ml as indicated. (FIG. 9J) Representative image from size exclusion chromatography for purification of IgA dimers for antibody BG1-20 (Tables 2 and 3). Y-axis displays absorption at 280 nm (mAU) and x-axis displays elution volume (in ml). (FIG. 9K) Representative Criterion™ TGX™ precast gel under non-reducing conditions after Coomassie Blue staining. Ladders with bands at indicated molecular weights are shown on the left and right end of the gel and adjacent to these are the pre-SEC sample and a monomeric IgA as indicated. The 22 lanes in the middle show consecutive elution fractions from SEC covering the multimer, dimer and monomer peaks as indicated and seen in (J). (FIG. 9L) Summary table for IgA neutralization of SARS-COV-2 pseudovirus. Shown are the IC50 and IC80 values in mg/ml (left two columns) followed by IC50 and IC80 values in nmol and IC50 and IC80 values in nmol corrected for the number of binding sites (Corr.) and the molar neutralization ratio (MNR) as defined by the IC50 of monomeric IgA divided by the IC50 for the dimeric version (Klein and Bjorkman, 2010). G-IgG1, mIgA=monomeric IgA, dIgA=dimeric IgA. (FIG. 9M) Bar graph summarizing the data from (L). Shown are all IgA antibodies which showed some level of neutralization in SARS-COV-2 pseudovirus neutralization assay in monomeric or dimeric form. Y-axis depicts IC50 (in nmol) corrected for the number of binding sites, x-axis indicates the different antibodies as indicated. Black bars show neutralization in IgG1 form (IgG), red bars in monomeric IgA (mIgA) and orange bars in dimeric IgA (dIgA) form.

FIGS. 10A-10H—BG10-19 cryo-EM validation, epitope mapping, and cross-reactive ELISA binding data (related to FIG. 3). (FIG. 10A) Representative micrograph and 2D class averages for BG10-19-S cryo-EM data collection. (FIG. 10B) Gold-standard FSC plot showing an overall resolution of 3.3 Å at FSC-0.143. (FIG. 10C) cryo-EM. (FIG. 10D) Local resolution estimation for the final cryo-EM reconstruction. (FIG. 10E) Structure of ACE2, RBD and BG10-19. (FIG. 10F) Sequence alignment of SARS-COV-2, SARS-COV, WIV1-COV, and SCH014-COV. RBD residues on the primary BG10-19 epitope are shaded blue. Residues contacted on the adjacent RBD are shaded orange. S309 epitope residues are also shown. (FIGS. 10G-10J) ELISA binding data for BG10-19, BG1-28, S309 (Pinto et al., 2020a), CR3022 (Tian et al., 2020), and the HIV-1 antibody IOMA (Gristick et al., 2016) against several sarbecoviruses.

FIGS. 11A-11H—Transcriptional Characteristics (related to FIG. 4). (FIG. 11A) Bar diagram showing the percentage of cells from individual donors contributing to all cells within each cluster as indicated. (FIG. 11B) Uniform Manifold Approximation and Projection (UMAP) plot displaying the cells defined by their single-cell transcriptome measurements. The cells are colored based on whether they were isolated with SARS-COV-2 S trimer (red) or RBD (turquoise) during cell sorting. (FIG. 11C) Same UMAP as in (FIG. 11B), but cells are colored based on their cell cycle phase G1 (red), G2M (green) or S (blue) as predicted by the Seurat cell cycle scoring function (Butler et al., 2018; Stuart et al., 2019). (FIG. 11D) Violin plots displaying the distribution of the expression levels of CD44, CXCR4 and CD38 (log 2) within each single-cell transcriptome cluster. (FIG. 11E) Bar plot displaying the percentage of each heavy chain isotype usage within each transcriptome cluster. (FIG. 11F) Box plots displaying the distribution of the number of inferred mutations in the heavy chains within each of the 6 transcriptome clusters. Mean values are displayed above each box. Significance of the difference between the mean values of each cluster with the mean of cluster 1 was calculated using one-tailed t-test. (FIG. 11G) Box plots displaying the distribution of the percentage of donor clonal cells within each transcriptome cluster (i.e. fraction of cells that are part of a B cell clone with 2 or more members across the entire donor). Significance of the difference between the mean values of clusters 3 and 4 with the mean of cluster 1 was calculated with one-tailed t-test. (FIG. 11H) Shannon entropy values calculated based on the general form of the diversity index proposed by (Hill, 1973), improved by resampling strategies in (Chao et al., 2014; Chao et al., 2015) and implemented in the Alakazam R package (Gupta et al., 2015). Each dot represents the Shannon entropy value of a specific donor among the 14 donors in the specified cluster. Shannon entropy values are reversely related with the clonal expansion within the clusters. Thus, according to the displayed comparisons TC1 contains the smallest clonal expansion. Significance values of pairwise comparisons are computed with two-tailed t-test. For (FIG. 11F), (FIG. 11G) and (FIG. 11H) red brackets indicate statistical significance of the difference between samples. *P<=0.05, ***P<=0.001, ****P<=0.0001 and n/s indicates no statistical significance.

FIGS. 12A-12D—TC3 and TC4 characterization (related to FIG. 4). (FIG. 12A) Violin plots displaying the distribution of the expression levels of various genes across single-cell transcriptome clusters. The significance of the mean value comparisons between different clusters was calculated using a two-tailed t-test. (FIG. 12B) Box plots displaying the percentage of VH3-53 or VH3-66 (with CDRH3 length smaller than 14 aa by IMGT definition (Lefranc et al., 2015)) cells of the 14 donors within each transcriptome cluster. Mean values of cluster 3 and 4 were compared with the mean values of other clusters using a two-tailed t-test. For (FIG. 12A) and (FIG. 12B) red brackets indicate statistical significance of the difference between samples. *P<=0.05, ***P<=0.001, ****P<=0.0001, and n/s indicates no statistical significance. (FIG. 12C) Scatter plots displaying the relationship between the percent of cells within each donor coming from TC0-5 with the neutralization titer values measured for each donor. Spearman correlation values with corresponding P values are shown on each subplot. Dots are colored based on the donor ID, as indicated. (FIG. 12D) Distribution of the percent of B-cell clones across 6 transcriptome clusters, stratified by neutralizing activity of the tested antibody. Each dot displays the percentage of cells from a specific clone (y-axis) that belongs to the specified transcriptome cluster (x-axis). Numbers on top of each violin plot show the median value of the distribution.

FIGS. 13A-13H—BG4-25-RBD structure and BG1-22-S trimer cryoEM validation (related to FIG. 5). (FIG. 13A) X-ray structure overview of BG4-25 (green) in complex with the SARS-COV-2 RBD (gray surface) and antibody CR3022 (yellow) (Tian et al., 2020). (FIG. 13B) BG4-25 CDR loops mapped on the surface of the RBD. Epitope residues are colored green and the ACE2 footprint shown (orange dotted line). (FIG. 13C) Bar diagram showing the mutation frequency of all inferred mutations that occurred in 10% or more of 93 isolated IgG VH3-53/VH3-66 antibodies with CDRH3 less than 14 amino acids (IMGT definition; (Lefranc et al., 2015)) from the collection of 6,113 SARS-COV-2 binding B cells and a reference group of 13,900 VH3-53/VH3-66 sequences from a human Ig gene repertoire set (see Methods and (Rubelt et al., 2012)). The frequencies of mutations in the antibodies from the SARS-COV-2 binding B cells are shown above the dotted line and the frequencies of mutations in the reference group are shown below the dotted line. (FIG. 13D, FIG. 13E) Residue level contacts between BG4-25 (green) and RBD (light gray). Potential H-bonds are shown as dashed lines. (FIG. 13F) Representative micrograph and 2D class averages for BG1-22-S cryo-EM data collection. (FIG. 13G) Gold-standard FSC plot showing an overall resolution of 3.7 Å at FSC-0.143. (FIG. 13H) Local resolution estimation for the final cryo-EM reconstruction.

FIGS. 14A-14I—Cryo-EM validation of BG7-15, BG7-20, and BG1-24-S trimer complexes (related to FIG. 6). Representative micrograph (scale bar=100 nm) selected from total dataset, 2D class averages, gold-standard FSC plots, and local resolution estimations for (FIGS. 14A-14C) BG7-15-S complex, (FIGS. 14D-14F) BG7-20-S complex, and (FIGS. 14G-14I) BG1-24-S complex.

FIG. 15—Schematic and results illustrating sorting of the receptor-binding domain and -trimer specific B cells from 24 individuals with likely SARS-COV-2 infection.

FIG. 16—There is a positive correlation between plasma neutralization titers and the fraction of IgG+ B cells in the SARS-COV-2-specific repertoire in 14 donors, consistent with the finding that most neutralizers stem from IgG+ B cells.

FIG. 17—Data showing that neutralization does not correlate with mutation levels but correlates with binding to SARS-COV-2 trimer in ELISA.

FIG. 18—Graphs showing that almost all neutralizing antibodies bind to the SARS-CoV-2 receptor-binding domain (RBD).

FIG. 19—Graphs showing that almost all neutralizing antibodies bind to the SARS-CoV-2 RBD.

FIG. 20—Graph showing that BG10-19 and BG1-28 show equal binding to SARS-CoV-2 and trimer/RBD.

FIG. 21—Table showing selected antibodies for structural characterization.

FIG. 22—Shows a 4.0 Å cryoEM structure of BG7-20-SARS-COV-2-S 6P complex. Dataset has preferential orientation issues, which will limit the ability to push the resolution much higher without collecting more data.

FIG. 23—Shows a 4.0 Å cryoEM structure of BG7-20-SARS-COV-2-S 6P complex. Data show two binding states: 2d/1u and 1d/2u bound by Fabs. The data look very similar to previously described C121-S structure (C121 is VH1-2/VL2-23).

FIG. 24—Shows a 3.5 Å cryoEM structure of BG10-19-SARS-COV-2-S 6P complex. Shows one main class that is symmetrical.

FIG. 25—Shows a 3.5 Å cryoEM structure of BG10-19-SARS-COV-2-S 6P complex. BG10-19 binds to an all down RBD conformation, with a main epitope focused near the N343-glycan. Interestingly, BG10-19 LC seems to make secondary contacts with adjacent RBD.

FIG. 26—Shows a 3.5 Å cryoEM structure of BG10-19-SARS-COV-2-S 6P complex . . . bridging RBDs on S trimers. BG10-19 HC makes contacts with primary epitope near N343-glycan, while the LC bridges adjacent down RBDs and interacts with residues that overlap with ACE2 receptor binding motif.

FIG. 27—Shows cryoEM reconstructions of BG1-22 and BG1-24-S complexes.

FIG. 28—Pie charts showing the total number of B cells sorted from each subject in the center of each pie and expanded B cell clones represented by pie slices. The white areas in each pie chart represent singlets. SARS-COV-2 surface trimer specific repertoire includes expanded B cell clones.

FIG. 29—Illustrates that SARS-COV-2 surface trimer-specific repertoire from 14 donors includes heavily expanded class of VH3-53/VH3-66 antibodies. These show a highly conserved sequence.

FIG. 30—UMAP plots showing that SARS-COV-2 surface trimer-specific repertoire from 14 donors falls into 5 distinct transcriptional clusters.

FIG. 31—Heat map showing that SARS-COV-2 surface trimer-specific repertoire from 14 donors falls into 5 distinct transcriptional clusters.

FIG. 32—Graphs showing that clusters 3 and 4 have higher levels of B cell activation markers such as CD80, CD40, and CD86.

FIG. 33—Graphs showing that VH3-53/VH3-66 with HC CDR3<14 amino acids are enriched in clusters 3 and 4.

FIG. 34—Pie chart and schematic illustrating the search for SARS/CoV-2/MERS cross-binding antibodies.

FIG. 35—Pie charts showing the total number of B cells sorted from each subject in the center of each pie and expanded B cell clones represented by pie slices. The white areas in each pie chart represent singlets. When used as sorting bait all 6 proteins yield differently expanded B cell clones.

FIG. 36—Pie charts showing that there is considerable clonal overlap between B cells sorted with the 6 different proteins.

FIG. 37—Venn diagrams summarizing all expanded clones and their immunoglobulin isotypes. The majority of expanded clones are IgM+ but some expanded clones include class-switched B cells.

FIG. 38—Graph illustrating that sorting with SARS/MERS/COV-2 trimers selects for antibodies that bind outside of the RBD (see, also, Table 7).

FIG. 39—Flow cytometry charts showing a pilot experiment for simultaneous staining with multiple coronavirus trimer single stains.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide cross-neutralizing coronavirus antibodies, in particular, cross-neutralizing SARS-COV2 antibodies capable of neutralizing a broad range of SARS-COV2 variants (e.g., BG10-19). SARS-COV2 poses a significant threat worldwide and monoclonal antibodies have been a focus in vaccine and therapeutic design to counteract the threat of SARS-COV-2. Here, Applicants combined SARS-COV-2-specific B cell sorting with single-cell VDJ and RNA-seq and structures of monoclonal antibodies to characterize B cell responses against SARS-COV-2. Applicants show that the SARS-COV-2-specific B cell repertoire consists of transcriptionally distinct B cell populations with potently neutralizing antibodies localized in two clusters that resemble memory and activated B cells. Of 92 isolated antibodies, 27 neutralize SARS-COV-2 with IC₈₀ values as low as 7 ng/ml Cryo-electron microscopy structures of potently-neutralizing antibody Fabs complexed with SARS-COV-2 spike trimers show recognition of various receptor-binding domain (RBD) epitopes, including one antibody (BG10-19) that locks the spike trimer in a completely closed conformation to potently neutralize SARS-COV-2, including lineages B.1.1.7 and B.1.351, SARS-COV and cross-reacts with heterologous RBDs. In certain embodiments, due to the epitopes targeted, the antibodies neutralize variants harboring mutations in the spike protein and RBD domains. Together, the results characterize transcriptional differences among SARS-COV-2-specific B cells and can inform vaccine and therapeutic development of cross-reactive antibodies, as well as, inform design of immunogens to elicit cross-reactive protection.

### Therapeutic Antibodies or Binding Fragments of an Antibody

### Antibodies

In certain embodiments, the present invention provides antibodies, antibody fragments, binding fragments of an antibody, or antigen binding fragments capable of binding to an antigen of interest (e.g., the receptor binding domain of SARS-COV-2 or the spike protein of SARS-COV-2). Applicants have identified antibodies from B cells that bound to specific bait proteins, i.e., trimers and RBD proteins from SARS-COV-2, SARS and MERS (see, Tables 2, 7 and 8). Table 8 provides a list of all nucleotide sequences identified in isolated B cells from 14 donors. The antibodies are not limited to the nucleotide sequences. The invention also includes antibodies comprising the amino acid sequences encoded for by the nucleotide sequences. One skilled in the art routinely converts nucleotide sequences to amino acid sequences. The disclosed antibodies may be cross-reactive to any coronavirus, thus providing for cross-reactive therapeutic antibodies. In certain embodiments, the antibodies provide epitopes for generating cross-reactive antibodies. In certain embodiments, the antibodies can be administered to treat a coronavirus infection, in particular SARS-COV-2 and variants thereof. In certain embodiments, the therapeutic antibodies described herein can be used as an initial treatment upon diagnosis or suspicion of a coronavirus infection, in particular a novel coronavirus infection. As used herein, “coronavirus” refers to enveloped viruses with a positive-sense single-stranded RNA genome and a nucleocapsid of helical symmetry that constitute the subfamily Orthocoronavirinae, in the family Coronaviridae (see, e.g., Woo P C, Huang Y, Lau S K, Yuen K Y. Coronavirus genomics and bioinformatics analysis. Viruses. 2010; 2(8):1804-1820).

In certain embodiments, the antibodies disclosed herein are cross-reactive to SARS-CoV-2 variants. As used herein, the term “variant” refers to any virus having one or more mutations as compared to a known virus. A strain is a genetic variant or subtype of a virus. The terms ‘strain’, ‘variant’, and ‘isolate’ may be used interchangeably. In certain embodiments, a variant has developed a “specific group of mutations” that causes the variant to behave differently than that of the strain it originated from.

While there are many thousands of variants of SARS-COV-2, (Koyama, Takahiko Koyama; Platt, Daniela; Parida, Laxmi (June 2020). “Variant analysis of SARS-COV-2 genomes”. Bulletin of the World Health Organization. 98: 495-504) there are also much larger groupings called clades. Several different clade nomenclatures for SARS-COV-2 have been proposed. As of December 2020, GISAID, referring to SARS-COV-2 as hCoV-19 identified seven clades (O, S, L, V, G, GH, and GR) (Alm E, Broberg E K, Connor T, et al. Geographical and temporal distribution of SARS-COV-2 clades in the WHO European Region, January to June 2020 [published correction appears in Euro Surveill. 2020 August; 25(33):]. Euro Surveill. 2020; 25(32):2001410). Also as of December 2020, Nextstrain identified five (19A, 19B, 20A, 20B, and 20C) (Cited in Alm et al. 2020). Guan et al. identified five global clades (G614, S84, V251, I378 and D392) (Guan Q, Sadykov M, Mfarrej S, et al. A genetic barcode of SARS-COV-2 for monitoring global distribution of different clades during the COVID-19 pandemic. Int J Infect Dis. 2020; 100:216-223). Rambaut et al. proposed the term “lineage” in a 2020 article in Nature Microbiology; as of December 2020, there have been five major lineages (A, B, B.1, B.1.1, and B.1.777) identified (Rambaut, A.; Holmes, E. C.; O'Toole, Á.; et al. “A dynamic nomenclature proposal for SARS-COV-2 lineages to assist genomic epidemiology”. 5: 1403-1407).

Genetic variants of SARS-COV-2 have been emerging and circulating around the world throughout the COVID-19 pandemic (see, e.g., The US Centers for Disease Control and Prevention; www.cdc.gov/coronavirus/2019-ncov/variants/variant-info.html). Exemplary, non-limiting variants applicable to the present disclosure include variants of SARS-COV-2, particularly those having substitutions of therapeutic concern. Table A shows exemplary, non-limiting genetic substitutions in SARS-COV-2 variants.

TABLE A

| | Common Pango Lineages with Spike |
|---|---|
| Spike Protein Substitution | Protein Substitutions |
| L452R | A.2.5, B.1, B.1.429, B.1.427, B.1.617.1, |
| | B.1.526.1, B.1.617.2, C.36.3 |
| E484K | B.1.1.318, B.1.1.7, B.1.351, B.1.525, |
| | B.1.526, B.1.621, B.1.623, P.1, P.1.1, |
| | P.1.2, R.1 |
| K417N, E484K, N501Y | B.1.351, B.1.351.3 |
| K417T, E484K, N501Y | P.1, P.1.1, P.1.2 |
| A67V, del69-70, T95I, del142-144, Y145D, del211, | B.1.1.529 and BA lineages |
| L212I, ins214EPE, G339D, S371L, S373P, S375F, | |
| K417N, N440K, G446S, S477N, T478K, E484A, | |
| Q493R, G496S, Q498R, N501Y, Y505H, T547K, | |
| D614G, H655Y, N679K, P681H, N764K, D796Y, | |
| N856K, Q954H, N969K, L981F | |

Phylogenetic Assignment of Named Global Outbreak (PANGO) Lineages is software tool developed by members of the Rambaut Lab. The associated web application was developed by the Centre for Genomic Pathogen Surveillance in South Cambridgeshire and is intended to implement the dynamic nomenclature of SARS-COV-2 lineages, known as the PANGO nomenclature. It is available at cov-lineages.org.

In some embodiments, the SARS-COV-2 variant is and/or includes: B.1.1.7, also known as Alpha (WHO) or UK variant, having the following spike protein substitutions: 69del, 70del, 144del, (E484K*), (S494P*), N501Y, A570D, D614G, P681H, T716I, S982A, and D1118H (K1191N*); B.1.351, also known as Beta (WHO) or South Africa variant, having the following spike protein substitutions: D80A, D215G, 241del, 242del, 243del, K417N, E484K, N501Y, D614G, and A701V; B.1.427, also known as Epsilon (WHO) or US California variant, having the following spike protein substitutions: L452R, and D614G; B.1.429, also known as Epsilon (WHO) or US California variant, having the following spike protein substitutions: S13I, W152C, L452R, and D614G; B.1.617.2, also known as Delta (WHO) or India variant, having the following spike protein substitutions: T19R, (G142D), 156del, 157del, R158G, L452R, T478K, D614G, P681R, and D950N; P.1, also known as Gamma (WHO) or Japan/Brazil variant, having the following spike protein substitutions: L18F, T20N, P26S, D138Y, R190S, K417T, E484K, N501Y, D614G, H655Y, and T1027I; and B.1.1.529 also known as Omicron (WHO), having the following spike protein substitutions: A67V, del69-70, T95I, del142-144, Y145D, del211, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, L981F, or any combination thereof.

In some embodiments, the SARS-COV-2 variant is classified and/or otherwise identified as a Variant of Concern (VOC) by the World Health Organization and/or the U.S. Centers for Disease Control. A VOC is a variant for which there is evidence of an increase in transmissibility, more severe disease (e.g., increased hospitalizations or deaths), significant reduction in neutralization by antibodies generated during previous infection or vaccination, reduced effectiveness of treatments or vaccines, or diagnostic detection failures.

In some embodiments, the SARS-Cov-2 variant is classified and/or otherwise identified as a Variant of High Consequence (VHC) by the World Health Organization and/or the U.S. Centers for Disease Control. A variant of high consequence has clear evidence that prevention measures or medical countermeasures (MCMs) have significantly reduced effectiveness relative to previously circulating variants.

In some embodiments, the SARS-Cov-2 variant is classified and/or otherwise identified as a Variant of Interest (VOI) by the World Health Organization and/or the U.S. Centers for Disease Control. A VOI is a variant with specific genetic markers that have been associated with changes to receptor binding, reduced neutralization by antibodies generated against previous infection or vaccination, reduced efficacy of treatments, potential diagnostic impact, or predicted increase in transmissibility or disease severity.

In some embodiments, the SARS-Cov-2 variant is classified and/or is otherwise identified as a Variant of Note (VON). As used herein, VON refers to both “variants of concern” and “variants of note” as the two phrases are used and defined by Pangolin (cov-lineages.org) and provided in their available “VOC reports” available at cov-lineages.org.

In some embodiments the SARS-Cov-2 variant is a VOC. In some embodiments, the SARS-COV-2 variant is or includes an Alpha variant (e.g., Pango lineage B.1.1.7), a Beta variant (e.g., Pango lineage B.1.351, B.1.351.1, B.1.351.2, and/or B.1.351.3), a Delta variant (e.g., Pango lineage B.1.617.2, AY.1, AY.2, AY.3 and/or AY.3.1); a Gamma variant (e.g., Pango lineage P.1, P.1.1, P.1.2, P.1.4, P.1.6, and/or P.1.7), a Omicon variant (B.1.1.529) or any combination thereof.

In some embodiments the SARS-Cov-2 variant is a VOI. In some embodiments, the SARS-COV-2 variant is or includes an Eta variant (e.g., Pango lineage B.1.525 (Spike protein substitutions A67V, 69del, 70del, 144del, E484K, D614G, Q677H, F888L)); an Iota variant (e.g., Pango lineage B.1.526 (Spike protein substitutions L5F, (D80G*), T95I, (Y144-*), (F157S*), D253G, (L452R*), (S477N*), E484K, D614G, A701V, (T859N*), (D950H*), (Q957R*))); a Kappa variant (e.g., Pango lineage B.1.617.1 (Spike protein substitutions (T95I), G142D, E154K, L452R, E484Q, D614G, P681R, Q1071H)); Pango lineage variant B.1.617.2 (Spike protein substitutions T19R, G142D, L452R, E484Q, D614G, P681R, D950N)), Lambda (e.g., Pango lineage C.37); or any combination thereof.

In some embodiments SARS-Cov-2 variant is a VON. In some embodiments, the SARS-Cov-2 variant is or includes Pango lineage variant P.1 (alias, B.1.1.28.1.) as described in Rambaut et al. 2020. Nat. Microbiol. 5:1403-1407) (spike protein substitutions: T20N, P26S, D138Y, R190S, K417T, E484K, N501Y, H655Y, TI027I)); an Alpha variant (e.g., Pango lineage B.1.1.7); a Beta variant (e.g., Pango lineage B.1.351, B.1.351.1, B.1.351.2, and/or B.1.351.3); Pango lineage variant B.1.617.2 (Spike protein substitutions T19R, G142D, L452R, E484Q, D614G, P681R, D950N)); an Eta variant (e.g., Pango lineage B.1.525); Pango lineage variant A.23.1 (as described in Bugembe et al. medRxiv. 2021. doi: doi.org/10.1101/2021.02.08.21251393) (spike protein substitutions: F157L, V367F, Q613H, P681R); or any combination thereof.

Structures have been determined for SARS-COV-2 variants and provide a structural framework for describing the impact of individual mutations on immune evasion (see, e.g., McCallum, et al., 2021, Molecular basis of immune evasion by the delta and kappa SARS-COV-2 variants, bioRxiv 2021.08.11.455956; doi.org/10.1101/2021.08.11.455956). The antibodies described herein can bind to epitopes on the structures of the variants described herein.

The term “antibody” is used interchangeably with the term “immunoglobulin” herein, and includes intact antibodies, fragments of antibodies, e.g., Fab, F(ab′)2 fragments, and intact antibodies and fragments that have been mutated either in their constant and/or variable region (e.g., mutations to produce chimeric, partially humanized, or fully humanized antibodies, as well as to produce antibodies with a desired trait, e.g., enhanced binding and/or reduced FcR binding). The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)2, Fabc, Fd, dAb, VHH and scFv and/or Fv fragments.

The term “antigen-binding fragment” refers to a polypeptide fragment of an immunoglobulin or antibody that binds antigen or competes with intact antibody (i.e., with the intact antibody from which they were derived) for antigen binding (i.e., specific binding). As such these antibodies or fragments thereof are included in the scope of the invention, provided that the antibody or fragment binds specifically to a target molecule.

In certain embodiments, the antibody or antibody fragment is a therapeutic antibody. In certain embodiments, the antibody is a neutralizing antibody. As used herein, “neutralizing antibody” refers to an antibody that is capable of neutralizing a pathogen or reducing infectivity, such as a viral pathogen (e.g., SARS-COV-2).

Applicants have identified specific antibodies capable of neutralizing SARS-COV-2, as well as neutralizing antibodies capable of binding SARS, MERS and CoV2 spike protein (Table 2). The antibodies identified specifically bind to the receptor binding domain (RBD) of SARS-CoV-2 spike or a region in the spike protein outside of the RBD. The antibodies neutralize pseudotyped virus expressing the spike protein. The antibodies include “complementarity determining regions” or “CDRs” interspersed among “frame regions” or “FRs”, as defined herein. As used herein CDRs refer to variable regions in an antibody that provide for antigen specificity. In certain embodiments, specific CDRs identified can be used in any antibody framework described further herein. In certain embodiments, one, two, or all three CDRs are used in a framework. In certain embodiments, CDR3 for the light and heavy chains are used in a framework. As used herein, framework can refer to an entire antibody where one or more variable chains or CDR sequences are substituted. It is common for therapeutic antibodies to use a common antibody constant region substituted with the identified variable chains or CDR sequences. In certain embodiments, frame region (FR) refers to the non-CDR regions or constant regions in the antibody.

In certain embodiments, antibodies prepared according to the present invention are substantially free of non-antibody protein. As used herein, a preparation of antibody protein having less than about 50% of non-antibody protein (also referred to herein as a “contaminating protein”), or of chemical precursors, is considered to be “substantially free.” 40%, 30%, 20%, 10% and more preferably 5% (by dry weight), of non-antibody protein, or of chemical precursors is considered to be substantially free. When the antibody protein or biologically active portion thereof is recombinantly produced, it is also preferably substantially free of culture medium, i.e., culture medium represents less than about 30%, preferably less than about 20%, more preferably less than about 10%, and most preferably less than about 5% of the volume or mass of the protein preparation.

In preferred embodiments, the antibodies of the present invention are monoclonal antibodies. As used herein, the term “monoclonal antibody” refers to a single antibody produced by any means, such as recombinant DNA technology. As used herein, the term “monoclonal antibody” also refers to an antibody derived from a clonal population of antibody-producing cells (e.g., B lymphocytes or B cells) which is homogeneous in structure and antigen specificity. The term “polyclonal antibody” refers to a plurality of antibodies originating from different clonal populations of antibody-producing cells which are heterogeneous in their structure and epitope specificity but which recognize a common antigen. Monoclonal and polyclonal antibodies may exist within bodily fluids, as crude preparations, or may be purified, as described herein.

The term “binding portion” of an antibody (or “antibody portion”) includes one or more complete domains, e.g., a pair of complete domains, as well as fragments of an antibody that retain the ability to specifically bind to a target molecule. It has been shown that the binding function of an antibody can be performed by fragments of a full-length antibody. Binding fragments are produced by recombinant DNA techniques, or by enzymatic or chemical cleavage of intact immunoglobulins. Binding fragments include Fab, Fab′, F(ab′)2, Fabc, Fd, dAb, Fv, single chains, VHH, single-chain antibodies, e.g., scFv, and single domain antibodies.

In certain embodiments, the antibodies described herein are humanized. In certain embodiments, “humanized” forms of non-human antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, FR residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin.

Examples of portions of antibodies or epitope-binding proteins encompassed by the present definition include: (i) the Fab fragment, having VL, CL, VH and CH1 domains; (ii) the Fab′ fragment, which is a Fab fragment having one or more cysteine residues at the C-terminus of the CH1 domain; (iii) the Fd fragment having VH and CH1 domains; (iv) the Fd′ fragment having VH and CH1 domains and one or more cysteine residues at the C-terminus of the CH1 domain; (v) the Fv fragment having the VL and VH domains of a single arm of an antibody; (vi) the dAb fragment (Ward et al., 341 Nature 544 (1989)) which consists of a VH domain or a VL domain that binds antigen; (vii) isolated CDR regions or isolated CDR regions presented in a functional framework; (viii) F(ab′)2 fragments which are bivalent fragments including two Fab′ fragments linked by a disulphide bridge at the hinge region; (ix) single chain antibody molecules (e.g., single chain Fv; scFv) (Bird et al., 242 Science 423 (1988); and Huston et al., 85 PNAS 5879 (1988)); (x) “diabodies” with two antigen binding sites, comprising a heavy chain variable domain (VH) connected to a light chain variable domain (VL) in the same polypeptide chain (see, e.g., EP 404,097; WO 93/11161; Hollinger et al., 90 PNAS 6444 (1993)); (xi) “linear antibodies” comprising a pair of tandem Fd segments (VH-Ch1-VH-Ch1) which, together with complementary light chain polypeptides, form a pair of antigen binding regions (Zapata et al., Protein Eng. 8(10): 1057-62 (1995); and U.S. Pat. No. 5,641,870).

In certain embodiments, the antibodies and CDRs of the present invention can be transferred to another antibody type to generate chimeric antibodies. It is intended that the term “antibody type” encompass any Ig class or any Ig subclass (e.g. the IgG1, IgG2, IgG3, and IgG4 subclassess of IgG) obtained from any source (e.g., humans and non-human primates, and in rodents, lagomorphs, caprines, bovines, equines, ovines, etc.).

The term “Ig class” or “immunoglobulin class”, as used herein, refers to the five classes of immunoglobulin that have been identified in humans and higher mammals, IgG, IgM, IgA, IgD, and IgE. The term “Ig subclass” refers to the two subclasses of IgM (H and L), three subclasses of IgA (IgA1, IgA2, and secretory IgA), and four subclasses of IgG (IgG1, IgG2, IgG3, and IgG4) that have been identified in humans and higher mammals. The antibodies can exist in monomeric or polymeric form; for example, IgM antibodies exist in pentameric form, and IgA antibodies exist in monomeric, dimeric or multimeric form.

The term “IgG subclass” refers to the four subclasses of immunoglobulin class IgG-IgG1, IgG2, IgG3, and IgG4 that have been identified in humans and higher mammals by the heavy chains of the immunoglobulins, V1-γ4, respectively. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen. The term “domain” refers to a globular region of a heavy or light chain polypeptide comprising peptide loops (e.g., comprising 3 to 4 peptide loops) stabilized, for example, by ß pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant” domains of an antibody light chain are referred to interchangeably as “light chain constant regions”, “light chain constant domains”, “CL” regions or “CL” domains. The “constant” domains of an antibody heavy chain are referred to interchangeably as “heavy chain constant regions”, “heavy chain constant domains”, “CH” regions or “CH” domains). The “variable” domains of an antibody light chain are referred to interchangeably as “light chain variable regions”, “light chain variable domains”, “VL” regions or “VL” domains). The “variable” domains of an antibody heavy chain are referred to interchangeably as “heavy chain constant regions”, “heavy chain constant domains”, “VH” regions or “VH” domains).

The term “region” can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “frame regions” or “FRs”, as defined herein.

The term “conformation” refers to the tertiary structure of a protein or polypeptide (e.g., an antibody, antibody chain, domain or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof.

“Specific binding” of an antibody means that the antibody exhibits appreciable affinity for a particular antigen or epitope and, generally, does not exhibit significant cross reactivity. “Appreciable” binding includes binding with an affinity of at least 25 μM. Antibodies with affinities greater than 1×10⁷ M⁻¹ (or a dissociation coefficient of 1 μM or less or a dissociation coefficient of 1 nm or less) typically bind with correspondingly greater specificity. Values intermediate of those set forth herein are also intended to be within the scope of the present invention and antibodies of the invention bind with a range of affinities, for example, 100 nM or less, 75 nM or less, 50 nM or less, 25 nM or less, for example 10 nM or less, 5 nM or less, 1 nM or less, or in embodiments 500 pM or less, 100 pM or less, 50 pM or less or 25 pM or less. An antibody that “does not exhibit significant crossreactivity” is one that will not appreciably bind to an entity other than its target (e.g., a different epitope or a different molecule). For example, an antibody that specifically binds to a target molecule will appreciably bind the target molecule but will not significantly react with non-target molecules or peptides. An antibody specific for a particular epitope will, for example, not significantly crossreact with remote epitopes on the same protein or peptide. Specific binding can be determined according to any art-recognized means for determining such binding. Preferably, specific binding is determined according to Scatchard analysis and/or competitive binding assays.

As used herein, the term “affinity” refers to the strength of the binding of a single antigen-combining site with an antigenic determinant. Affinity depends on the closeness of stereochemical fit between antibody combining sites and antigen determinants, on the size of the area of contact between them, on the distribution of charged and hydrophobic groups, etc. Antibody affinity can be measured by equilibrium dialysis or by the kinetic BIACORE™ method. The dissociation constant, Kd, and the association constant, Ka, are quantitative measures of affinity.

In certain embodiments, the antibodies described herein or identified according to the methods described herein are blocking antibodies. As used herein, a “blocking” antibody or an antibody “antagonist” is one which inhibits or reduces biological activity of the antigen(s) it binds. In certain embodiments, the blocking antibodies or antagonist antibodies or portions completely inhibit the biological activity of the antigen(s). For example, the present invention includes antibodies which disrupt receptor/ligand interactions either partially or fully.

### Therapeutic Antibody Modifications

In certain example embodiments, the therapeutic antibodies of the present invention may be modified, such that they acquire advantageous properties for therapeutic use (e.g., stability and specificity), but maintain their biological activity. Therapeutic antibodies may be modified to increase stability or to provide characteristics that improve efficacy of the antibody when administered to a subject in vivo. As used herein in reference to therapeutic antibodies, the terms “modified”, “modification” and the like refer to one or more changes that enhance a desired property of the therapeutic antibody. “Modification” includes a covalent chemical modification that does not alter the primary amino acid sequence of the therapeutic antibody itself. Such desired properties include, for example, prolonging the in vivo half-life, increasing the stability, reducing the clearance, altering the immunogenicity or allergenicity, or cellular targeting. Changes to a therapeutic antibody that may be carried out include, but are not limited to, conjugation to a carrier protein, conjugation to a ligand, conjugation to another antibody, PEGylation, polysialylation HESylation, recombinant PEG mimetics, Fc fusion, albumin fusion, nanoparticle attachment, nanoparticulate encapsulation, cholesterol fusion, iron fusion, acylation, amidation, glycosylation, side chain oxidation, phosphorylation, biotinylation, the addition of a surface active material, the addition of amino acid mimetics, or the addition of unnatural amino acids. Modified therapeutic antibodies also include analogs. By “analog” is meant a molecule that is not identical, but has analogous functional or structural features. For example, a therapeutic antibody analog retains the biological activity of a corresponding antibody, while having certain biochemical modifications that enhance the analog's function relative to another antibody. Such biochemical modifications could increase the analog's protease resistance, membrane permeability, or half-life, without altering, for example, antigen binding. An analog may include an unnatural amino acid.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Modified antibodies (e.g., fusion proteins) may include a spacer or a linker. The terms “spacer” or “linker” as used in reference to a fusion protein refers to a peptide that joins the proteins comprising a fusion protein. Generally, a spacer has no specific biological activity other than to join or to preserve some minimum distance or other spatial relationship between the proteins. However, in certain embodiments, the constituent amino acids of a spacer may be selected to influence some property of the molecule such as the folding, net charge, or hydrophobicity of the molecule. Suitable linkers for use in an embodiment of the present invention are well known to those of skill in the art and include, but are not limited to, straight or branched-chain carbon linkers, heterocyclic carbon linkers, or peptide linkers. The linker is used to separate two peptides by a distance sufficient to ensure that, in a preferred embodiment, each peptide properly folds. Preferred peptide linker sequences adopt a flexible extended conformation and do not exhibit a propensity for developing an ordered secondary structure. Typical amino acids in flexible protein regions include Gly, Asn and Ser. Virtually any permutation of amino acid sequences containing Gly, Asn and Ser would be expected to satisfy the above criteria for a linker sequence. Other near neutral amino acids, such as Thr and Ala, also may be used in the linker sequence. Still other amino acid sequences that may be used as linkers are disclosed in Maratea et al. (1985), Gene 40: 39-46; Murphy et al. (1986) Proc. Nat'l. Acad. Sci. USA 83: 8258-62; U.S. Pat. Nos. 4,935,233; and 4,751,180.

The clinical effectiveness of protein therapeutics (e.g., antibodies) is often limited by short plasma half-life and susceptibility to protease degradation. Studies of various therapeutic proteins (e.g., filgrastim) have shown that such difficulties may be overcome by various modifications, including conjugating or linking the polypeptide sequence to any of a variety of non-proteinaceous polymers, e.g., polyethylene glycol (PEG), polypropylene glycol, or polyoxyalkylenes (see, for example, typically via a linking moiety covalently bound to both the protein and the nonproteinaceous polymer, e.g., a PEG).

It is well known that the properties of certain proteins can be modulated by attachment of polyethylene glycol (PEG) polymers, which increases the hydrodynamic volume of the protein and thereby slows its clearance by kidney filtration. (See, e.g., Clark et al., J. Biol. Chem. 271: 21969-21977 (1996)). Such PEG-conjugated biomolecules have been shown to possess clinically useful properties, including better physical and thermal stability, protection against susceptibility to enzymatic degradation, increased solubility, longer in vivo circulating half-life and decreased clearance, reduced immunogenicity and antigenicity, and reduced toxicity. Therefore, it is envisioned that certain agents can be PEGylated (e.g., on peptide residues) to provide enhanced therapeutic benefits such as, for example, increased efficacy by extending half-life in vivo. In certain embodiments, PEGylation of the agents may be used to extend the serum half-life of the agents and allow for particular agents to be capable of crossing the blood-brain barrier. Thus, in one embodiment, PEGylating antibodies improve the pharmacokinetics and pharmacodynamics of the antibodies.

In regards to peptide PEGylation methods, reference is made to Lu et al., Int. J. Pept. Protein Res. 43: 127-38 (1994); Lu et al., Pept. Res. 6: 140-6 (1993); Felix et al., Int. J. Pept. Protein Res. 46: 253-64 (1995); Gaertner et al., Bioconjug. Chem. 7: 38-44 (1996); Tsutsumi et al., Thromb. Haemost. 77: 168-73 (1997); Francis et al., hit. J. Hematol. 68: 1-18 (1998); Roberts et al., J. Pharm. Sci. 87: 1440-45 (1998); and Tan et al., Protein Expr. Purif. 12: 45-52 (1998). Polyethylene glycol or PEG is meant to encompass any of the forms of PEG that have been used to derivatize other proteins, including, but not limited to, mono-(C1-10) alkoxy or aryloxy-polyethylene glycol. Suitable PEG moieties include, for example, 40 kDa methoxy poly(ethylene glycol) propionaldehyde (Dow, Midland, Mich.); 60 kDa methoxy poly(ethylene glycol) propionaldehyde (Dow, Midland, Mich.); 40 kDa methoxy poly(ethylene glycol) maleimido-propionamide (Dow, Midland, Mich.); 31 kDa alpha-methyl-w-(3-oxopropoxy), polyoxyethylene (NOF Corporation, Tokyo); mPEG2-NHS-40k (Nektar); mPEG2-MAL-40k (Nektar), SUNBRIGHT GL2-400MA ((PEG)240 kDa) (NOF Corporation, Tokyo), SUNBRIGHT ME-200MA (PEG20 kDa) (NOF Corporation, Tokyo). The PEG groups are generally attached to the peptide (e.g., RBD) via acylation or alkylation through a reactive group on the PEG moiety (for example, a maleimide, an aldehyde, amino, thiol, or ester group) to a reactive group on the peptide (for example, an aldehyde, amino, thiol, a maleimide, or ester group).

The PEG molecule(s) may be covalently attached to any Lys, Cys, or K(CO(CH2)2SH) residues at any position in a peptide. In certain embodiments, the antibodies described herein can be PEGylated directly to any amino acid at the N-terminus by way of the N-terminal amino group. A “linker arm” may be added to a peptide to facilitate PEGylation. PEGylation at the thiol sidechain of cysteine has been widely reported (see, e.g., Caliceti & Veronese, Adv. Drug Deliv. Rev. 55: 1261-77 (2003)). If there is no cysteine residue in the peptide, a cysteine residue can be introduced through substitution or by adding a cysteine to the N-terminal amino acid. In certain embodiments, proteins are PEGylated through the side chains of a cysteine residue added to the N-terminal amino acid.

In exemplary embodiments, the PEG molecule(s) may be covalently attached to an amide group in the C-terminus of a peptide. In certain embodiments, the PEG molecule used in modifying an agent of the present invention is branched while in other embodiments, the PEG molecule may be linear. In particular aspects, the PEG molecule is between 1 kDa and 100 kDa in molecular weight. In further aspects, the PEG molecule is selected from 10, 20, 30, 40, 50, 60, and 80 kDa. In further still aspects, it is selected from 20, 40, or 60 kDa. Where there are two PEG molecules covalently attached to the agent of the present invention, each is 1 to 40 kDa and in particular aspects, they have molecular weights of 20 and 20 kDa, 10 and 30 kDa, 30 and 30 kDa, 20 and 40 kDa, or 40 and 40 kDa. In particular aspects, the antibodies contain mPEG-cysteine. The mPEG in mPEG-cysteine can have various molecular weights. The range of the molecular weight is preferably 5 kDa to 200 kDa, more preferably 5 kDa to 100 kDa, and further preferably 20 kDa to 60 kDA. The mPEG can be linear or branched.

The present disclosure also contemplates the use of PEG Mimetics. Recombinant PEG mimetics have been developed that retain the attributes of PEG (e.g., enhanced serum half-life) while conferring several additional advantageous properties. By way of example, simple polypeptide chains (comprising, for example, Ala, Glu, Gly, Pro, Ser and Thr) capable of forming an extended conformation similar to PEG can be produced recombinantly already fused to the antibodies (e.g., Amunix' XTEN technology; Mountain View, CA). This obviates the need for an additional conjugation step during the manufacturing process. Moreover, established molecular biology techniques enable control of the side chain composition of the polypeptide chains, allowing optimization of immunogenicity and manufacturing properties.

Glycosylation can dramatically affect the physical properties of proteins and can also be important in protein stability, secretion, and subcellular localization (see, e.g., Solá and Griebenow, Glycosylation of Therapeutic Proteins: An Effective Strategy to Optimize Efficacy. BioDrugs. 2010; 24(1): 9-21). Proper glycosylation can be essential for biological activity. In fact, some genes from eukaryotic organisms, when expressed in bacteria (e.g., E. coli) which lack cellular processes for glycosylating proteins, yield proteins that are recovered with little or no activity by virtue of their lack of glycosylation. For purposes of the present disclosure, “glycosylation” is meant to broadly refer to the enzymatic process that attaches glycans to proteins, lipids or other organic molecules. The use of the term “glycosylation” in conjunction with the present disclosure is generally intended to mean adding or deleting one or more carbohydrate moieties (either by removing the underlying glycosylation site or by deleting the glycosylation by chemical and/or enzymatic means), and/or adding one or more glycosylation sites that may or may not be present in the original sequence.

Addition of glycosylation sites can be accomplished by altering the amino acid sequence. The alteration to the polypeptide may be made, for example, by the addition of, or substitution by, one or more serine or threonine residues (for O-linked glycosylation sites) or asparagine residues (for N-linked glycosylation sites). The structures of N-linked and O-linked oligosaccharides and the sugar residues found in each type may be different. One type of sugar that is commonly found on both is N-acetylneuraminic acid (hereafter referred to as sialic acid). Sialic acid is usually the terminal residue of both N-linked and O-linked oligosaccharides and, by virtue of its negative charge, may confer acidic properties to the glycoprotein. A particular embodiment of the present disclosure comprises the generation and use of N-glycosylation variants.

The present disclosure also contemplates the use of polysialylation, the conjugation of peptides and proteins to the naturally occurring, biodegradable a-(2→8) linked polysialic acid (“PSA”) in order to improve their stability and in vivo pharmacokinetics. PSA is a biodegradable, non-toxic natural polymer that is highly hydrophilic, giving it a high apparent molecular weight in the blood which increases its serum half-life. In addition, polysialylation of a range of peptide and protein therapeutics has led to markedly reduced proteolysis, retention of activity in vivo activity, and reduction in immunogenicity and antigenicity (see, e.g., G. Gregoriadis et al., Int. J. Pharmaceutics 300(1-2): 125-30). As with modifications with other conjugates (e.g., PEG), various techniques for site-specific polysialylation are available (see, e.g., T. Lindhout et al., PNAS 108(18)7397-7402 (2011)).

Additional suitable components and molecules for conjugation include, for example, thyroglobulin; albumins such as human serum albumin (HAS); tetanus toxoid; Diphtheria toxoid; polyamino acids such as poly(D-lysine:D-glutamic acid); VP6 polypeptides of rotaviruses; influenza virus hemaglutinin, influenza virus nucleoprotein; Keyhole Limpet Hemocyanin (KLH); and hepatitis B virus core protein and surface antigen; or any combination of the foregoing.

Fusion of albumin to one or more antibodies of the present disclosure can, for example, be achieved by genetic manipulation, such that the DNA coding for HSA, or a fragment thereof, is joined to the DNA coding for the one or more antibodies. Albumin itself may be modified to extend its circulating half-life. Fusion of the modified albumin to one or more polypeptides can be attained by the genetic manipulation techniques described above or by chemical conjugation; the resulting fusion molecule has a half-life that exceeds that of fusions with non-modified albumin. (See WO2011/051489).

Several albumin-binding strategies have been developed as alternatives for direct fusion, including albumin binding through a conjugated fatty acid chain (acylation). Because serum albumin is a transport protein for fatty acids, these natural ligands with albumin-binding activity have been used for half-life extension of small protein therapeutics. For example, insulin determir (LEVEMIR), an approved product for diabetes, comprises a myristyl chain conjugated to a genetically-modified insulin, resulting in a long-acting insulin analog.

Another type of modification is to conjugate (e.g., link) one or more additional components or molecules at the N- and/or C-terminus of a polypeptide sequence, such as another protein, or a carrier molecule. Thus, an exemplary polypeptide sequence can be provided as a conjugate with another component or molecule. A conjugate modification may result in a polypeptide sequence that retains activity with an additional or complementary function or activity of the second molecule. For example, a polypeptide sequence may be conjugated to a molecule, e.g., to facilitate solubility, storage, in vivo or shelf half-life or stability, reduction in immunogenicity, delayed or controlled release in vivo, etc. Other functions or activities include a conjugate that reduces toxicity relative to an unconjugated polypeptide sequence, a conjugate that targets a type of cell or organ more efficiently than an unconjugated polypeptide sequence, or a drug to further counter the causes or effects associated with a disorder or disease as set forth herein.

The present disclosure contemplates the use of other modifications, currently known or developed in the future, of the polypeptides to improve one or more properties. One such method for prolonging the circulation half-life, increasing the stability, reducing the clearance, or altering the immunogenicity or allergenicity of a polypeptide of the present disclosure involves modification of the polypeptide sequences by hesylation, which utilizes hydroxyethyl starch derivatives linked to other molecules in order to modify the molecule's characteristics. Various aspects of hesylation are described in, for example, U.S. Patent Appln. Nos. 2007/0134197 and 2006/0258607.

In particular embodiments, the antibodies include a protecting group covalently joined to the N-terminal amino group. In exemplary embodiments, a protecting group covalently joined to the N-terminal amino group of the proteins reduces the reactivity of the amino terminus under in vivo conditions. Amino protecting groups include —C1-10 alkyl, —C1-10 substituted alkyl, —C2-10 alkenyl, —C2-10 substituted alkenyl, aryl, —C1-6 alkyl aryl, —C(O)—(CH2)1-6-COOH, —C(O)—C1-6 alkyl, —C(O)-aryl, —C(O)—O-C1-6 alkyl, or —C(O)—O-aryl. In particular embodiments, the amino terminus protecting group is selected from the group consisting of acetyl, propyl, succinyl, benzyl, benzyloxycarbonyl, and t-butyloxycarbonyl. In other embodiments, deamination of the N-terminal amino acid is another modification that may be used for reducing the reactivity of the amino terminus under in vivo conditions.

Chemically modified compositions of the antibodies wherein the antibody is linked to a polymer are also included within the scope of the present invention. The polymer selected is usually modified to have a single reactive group, such as an active ester for acylation or an aldehyde for alkylation, so that the degree of polymerization may be controlled. Included within the scope of polymers is a mixture of polymers. Preferably, for therapeutic use of the end-product preparation, the polymer will be pharmaceutically acceptable. The polymer or mixture thereof may include but is not limited to polyethylene glycol (PEG), monomethoxy-polyethylene glycol, dextran, cellulose, or other carbohydrate-based polymers, poly-(N-vinyl pyrrolidone) polyethylene glycol, propylene glycol homopolymers, a polypropylene oxide/ethylene oxide co-polymer, polyoxyethylated polyols (for example, glycerol), and polyvinyl alcohol.

In other embodiments, the antibodies are modified by PEGylation, cholesterylation, or palmitoylation. The modification can be to any amino acid residue. In preferred embodiments, the modification is to the N-terminal amino acid of the antibodies, either directly to the N-terminal amino acid or by way coupling to the thiol group of a cysteine residue added to the N-terminus or a linker added to the N-terminus such as trimesoyl tris(3,5-dibromosalicylate (Ttds). In certain embodiments, the N-terminus of the antibodies comprise a cysteine residue to which a protecting group is coupled to the N-terminal amino group of the cysteine residue and the cysteine thiolate group is derivatized with N-ethylmaleimide, PEG group, cholesterol group, or palmitoyl group. In other embodiments, an acetylated cysteine residue is added to the N-terminus of the agents, and the thiol group of the cysteine is derivatized with N-ethylmaleimide, PEG group, cholesterol group, or palmitoyl group. In certain embodiments, the antibodies of the present invention consist of an amino acid sequence which is bound with a methoxypolyethylene glycol(s) via a linker.

Substitutions of amino acids may be used to modify an antibody of the present invention. The phrase “substitution of amino acids” as used herein encompasses substitution of amino acids that are the result of both conservative and non-conservative substitutions. Conservative substitutions are the replacement of an amino acid residue by another similar residue in a polypeptide. Typical but not limiting conservative substitutions are the replacements, for one another, among the aliphatic amino acids Ala, Val, Leu and Ile; interchange of Ser and Thr containing hydroxy residues, interchange of the acidic residues Asp and Glu, interchange between the amide-containing residues Asn and Gln, interchange of the basic residues Lys and Arg, interchange of the aromatic residues Phe and Tyr, and interchange of the small-sized amino acids Ala, Ser, Thr, Met, and Gly. Non-conservative substitutions are the replacement, in a polypeptide, of an amino acid residue by another residue which is not biologically similar. For example, the replacement of an amino acid residue with another residue that has a substantially different charge, a substantially different hydrophobicity, or a substantially different spatial configuration.

In certain embodiments, antibodies of the present invention are modified by affinity maturation. Affinity maturation refers to the introduction of random mutations across the full length of selected CDR or variable chains and screening for increased affinity or neutralization activity. In certain embodiments, phage display or ribosome display is used to produce antibodies with increased affinity for the antigen. The in vitro affinity maturation has successfully been used to optimize antibodies, antibody fragments or other peptide molecules like antibody mimetics. Random mutations inside the CDRs are introduced using radiation, chemical mutagens or error-prone PCR. In addition, the genetic diversity can be increased by chain shuffling. Two or three rounds of mutation and selection using display methods like phage display usually results in antibody fragments with affinities in the low nanomolar range (see, e.g., Roskos L.; Klakamp S.; Liang M.; Arends R.; Green L. (2007). Stefan Dübel (ed.). Handbook of Therapeutic Antibodies. Weinheim: Wiley-VCH. pp. 145-169).

One of skill in the art from this disclosure and the knowledge in the art will appreciate that there are a variety of ways in which to produce such therapeutic antibodies. In general, such therapeutic antibodies may be produced either in vitro or in vivo. Therapeutic antibodies may be produced in vitro as peptides or polypeptides, which may then be formulated into a pharmaceutical composition and administered to a subject. Such in vitro production may occur by a variety of methods known to one of skill in the art such as, for example, peptide synthesis or expression of a peptide/polypeptide from a DNA or RNA molecule in any of a variety of bacterial, eukaryotic, or viral recombinant expression systems, followed by purification of the expressed antibodies (e.g., with protein A or G). Alternatively, antibodies may be produced in vivo by introducing molecules (e.g., DNA, RNA, viral expression systems, and the like) that encode antibodies into a subject, whereupon the encoded therapeutic antibodies are expressed.

In certain embodiments, the antibodies as defined for the present invention include derivatives that are modified, i.e., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

### Administration of Therapeutic Antibodies

For therapeutic uses, the antibodies described herein may be administered systemically, for example, formulated in a pharmaceutically-acceptable buffer such as physiological saline. Preferable routes of administration include, for example, subcutaneous, intravenous, interperitoneal, intramuscular, or intradermal injections that provide continuous, sustained levels of the antibody in the patient. Treatment of human patients or other animals will be carried out using a therapeutically effective amount of a therapeutic identified herein in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of the neoplasia. Generally, amounts will be in the range of those used for other agents used in the treatment of other diseases associated with neoplasia, although in certain instances lower amounts will be needed because of the increased specificity of the compound. For example, a therapeutic compound is administered at a dosage that is cytotoxic to a neoplastic cell.

Human dosage amounts can initially be determined by extrapolating from the amount of antibody used in mice, as a skilled artisan recognizes it is routine in the art to modify the dosage for humans compared to animal models. Of course, this dosage amount may be adjusted upward or downward, as is routinely done in such treatment protocols, depending on the results of the initial clinical trials and the needs of a particular patient.

The therapeutic regimens disclosed herein comprise administration of antibodies of the invention or pharmaceutical compositions thereof to the patient in a single dose or in multiple doses (e.g., 1, 2, 3, 4, 5, 6, 7, 8, 10, 15, 20, or more doses). In one aspect, the therapeutic regimens comprise administration of the antibodies of the invention or pharmaceutical compositions thereof in multiple doses. When administered in multiple doses, the antibodies are administered with a frequency and in an amount sufficient to treat SARS-COV-2. For example, the frequency of administration ranges from once a day up to about four times a day. In another example, the frequency of administration ranges from about once a week up to about once every six weeks.

Significant progress has been made in understanding pharmacokinetics (PK), pharmacodynamics (PD), as well as toxicity profiles of therapeutic antibodies in animals and humans, which have been in commercial development for more than three decades (see, e.g., Vugmeyster et al., Pharmacokinetics and toxicology of therapeutic proteins: Advances and challenges, World J Biol Chem. 2012 Apr. 26; 3(4): 73-92). In certain embodiments, therapeutic antibodies are administered by parenteral routes, such as intravenous (IV), subcutaneous (SC) or intramuscular (IM) injection. Molecular size, hydrophilicity, and gastric degradation are the main factors that preclude gastrointestinal (GI) absorption of therapeutic proteins (see, e.g., Keizer, et al., Clinical pharmacokinetics of therapeutic monoclonal antibodies. Clin Pharmacokinet. 2010 August; 49(8):493-507). Pulmonary delivery with aerosol formulations or dry powder inhalers has been used for selected proteins, e.g., exubera™ (see, e.g., Scheuch and Siekmeier, Novel approaches to enhance pulmonary delivery of proteins and peptides. J Physiol Pharmacol. 2007 November; 58 Suppl 5(Pt 2):615-25). Intravitreal injections have been used for peptides and proteins that require only local activity (see, e.g., Suresh, et al., Ocular Delivery of Peptides and Proteins. In: Van Der Walle C., editor. Peptide and Protein Delivery. London: Academic Press; 2011. pp. 87-103). In certain embodiments, SC administration of therapeutic antibodies is often a preferred route. In particular, the suitability of SC dosing for self-administration translates into significantly reduced treatment costs.

The pharmaceutical composition may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

As indicated above, the pharmaceutical compositions according to the invention may be in the form suitable for sterile injection. To prepare such a composition, the suitable antibodies are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate).

### Vector Delivery

The invention also provides a delivery system comprising one or more vectors or one or more polynucleotide molecules, the one or more vectors or polynucleotide molecules comprising one or more polynucleotide molecules encoding an antibody of the present invention.

In general, and throughout this specification, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Vectors for and that result in expression in a eukaryotic cell can be referred to herein as “eukaryotic expression vectors.” Common expression vectors of utility in recombinant DNA techniques are often in the form of plasmids.

### Identification of Neutralizing Antibodies

Applicants identified specific gene signatures present in B cells that produced neutralizing antibodies (clusters 3 and 4 of Table 4). In certain embodiments, neutralizing antibodies are identified by first enriching for B cells expressing the signature. In certain embodiments, cell sorting using antibodies specific for surface markers in the signatures are used to enrich for the cell types. In certain embodiments, fluorescence-activated cell sorting (FACS) can be used to enrich for the B cells. In certain embodiments, magnetic beads specific for surface markers can be used to enrich for the B cells. In certain embodiments, B cells are enriched from a subject infected with the pathogen (e.g., virus). Antibodies for any pathogen may be identified by enriching for B cells having the disclosed signatures followed by sequencing of the antibody sequences from the enriched cells.

In certain embodiments, B cells are obtained from a unit of blood collected from a subject using any number of techniques known to the skilled artisan, such as Ficoll separation. In one preferred embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). B cells can be further isolated by positive or negative selection. A preferred method is cell sorting and/or selection via magnetic immunoadherence or flow cytometry that uses a cocktail of monoclonal antibodies directed to cell surface markers present on the cells positively or negatively selected. Such separation can be performed using standard methods available in the art. For example, any magnetic separation methodology may be used including a variety of which are commercially available, (e.g., DYNAL® Magnetic Particle Concentrator (DYNAL MPC®)).

In certain embodiments, the method includes assays capable of validating antibody binding and neutralization activity. In certain embodiments, the validation assay is an immunoassay. Immunoassay methods are based on the reaction of an antibody to its corresponding target or analyte and can detect the analyte in a sample depending on the specific assay format. Immunoassays have been designed for use with a wide range of biological sample matrices Immunoassay formats have been designed to provide qualitative, semi-quantitative, and quantitative results. Quantitative results may be generated by determining the concentration of analyte detected by an antibody.

Numerous immunoassay formats have been designed. Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. ELISA or EIA can be quantitative for the detection of an analyte/biomarker. This method relies on attachment of a label to either the analyte or the antibody and the label component includes, either directly or indirectly, an enzyme. ELISA tests may be formatted for direct, indirect, competitive, or sandwich detection of the analyte. Additional techniques include, for example, agglutination, nephelometry, turbidimetry, Western blot, immunoprecipitation, immunocytochemistry, immunohistochemistry, flow cytometry, Luminex assay, and others (see ImmunoAssay: A Practical Guide, edited by Brian Law, published by Taylor & Francis, Ltd., 2005 edition).

Viral neutralizing assays can be used to validate identified antibody frameworks. In certain embodiments, the assay uses live virus. In certain embodiments, the assay uses pseudotyped virus particles (see, e.g., Gentili, 2015). In certain embodiments, the neutralizing assay is performed without live virus (see, e.g., Tan, C. W., Chia, W. N., Qin, X. et al. A SARS-CoV-2 surrogate virus neutralization test based on antibody-mediated blockage of ACE2-spike protein-protein interaction. Nat Biotechnol 38, 1073-1078 (2020).

Further embodiments are illustrated in the following Examples which are given for illustrative purposes only and are not intended to limit the scope of the invention.

### EXAMPLES

### Example 1—B Cell Genomics and Structural Studies Uncover Cross-Neutralizing SARS-COV-2 Antibodies

Applicants used scRNA-Seq to investigate SARS-COV-2 spike-specific B cell responses in 14 subjects who had recovered from COVID-19. By matching the VDJ sequence and transcriptional profile with neutralization and binding studies from 92 monoclonal antibodies produced from single B cells, Applicants identified two transcriptional clusters that are enriched in B cells that produce binding and neutralizing antibodies. Six of the most potently neutralizing antibodies in the collection derived from B cells in these two transcriptional clusters were structurally characterized. Applicants found that they adopt distinct RBD-binding orientations, including the BG10-19 antibody that reaches between adjacent RBDs on the protomers of a single spike trimer, resulting in a locked spike trimer conformation that cannot bind ACE2. BG10-19 binds adjacent RBDs in a manner distinct from previously-described antibodies that lock the trimer into a closed conformation (Barnes et al., 2020a; Tortorici, 2020). It recognizes SARS-COV-2, SARS-COV, and zoonotic RBDs, and demonstrated potent neutralization of SARS-COV-2, the UK variant B.1.1.7 (Davies et al., 2021), the South African variant B.1.351 (Tegally et al., 2020) as well as the heterologous SARS-COV pseudotyped viruses. Furthermore, characterization of antibodies belonging to the VH3-53/VH3-66-encoded class (Barnes et al., 2020b; Wu et al., 2020a; Yuan et al., 2020) showed common binding modes for antibodies with short (<14 amino acids) and long (>15 amino acids) CDRH3 loops, providing new insights into this recurring class of SARS-COV-2 neutralizing antibodies.

A cohort of recently recovered COVID-19 patients shows serum neutralizing activity. To understand the development of B cell responses after SARS-COV-2 infection, Applicants enrolled 14 subjects who had recently recovered from COVID-19. All subjects were diagnosed in March 2020, none required hospitalization, and the time between diagnosis and enrollment of subjects ranged between 31 and 61 days with a median of 51 days (Table 1). 12 of 14 subjects were diagnosed with COVID-19 using PCR-based testing at the time of presentation. The remaining two subjects were diagnosed based on serum reactivity to RBD in ELISA, clinical symptoms and history of recent exposure (Table 1). All subjects had serum antibodies against RBD in ELISA (Table 1).

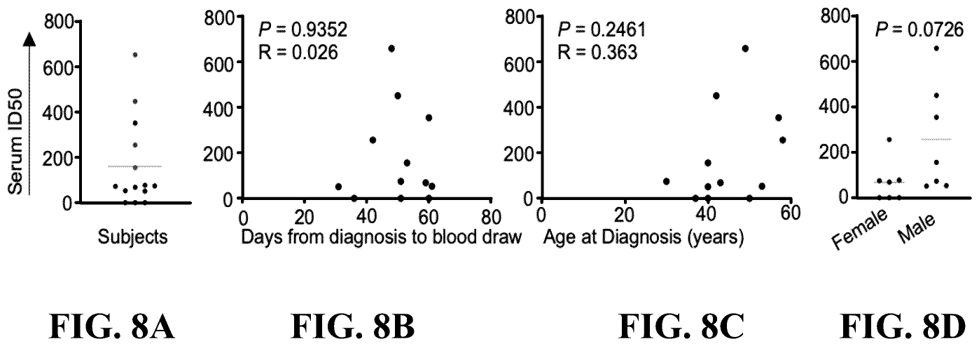

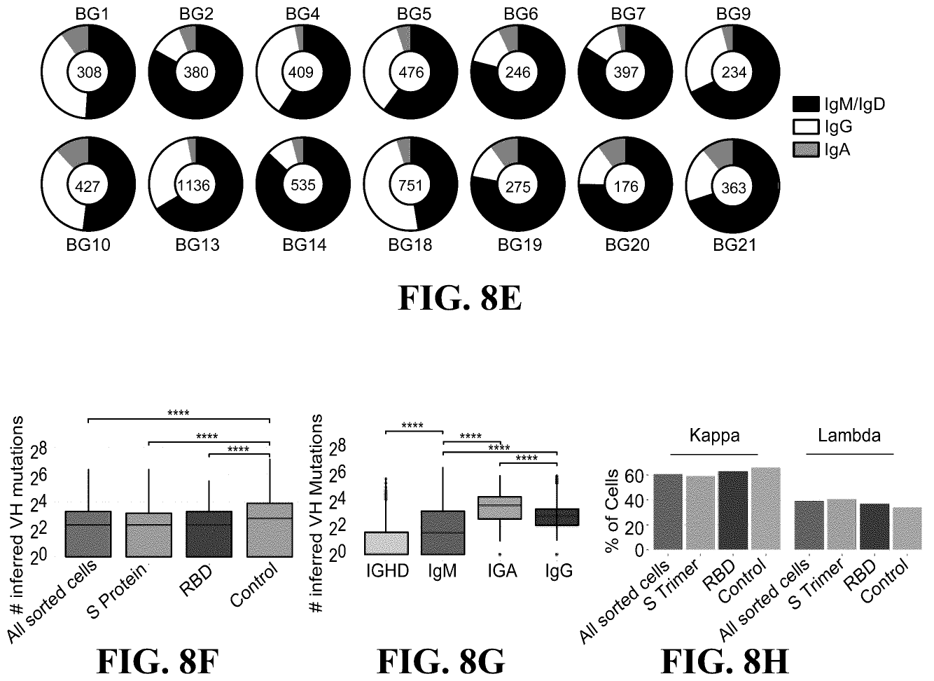

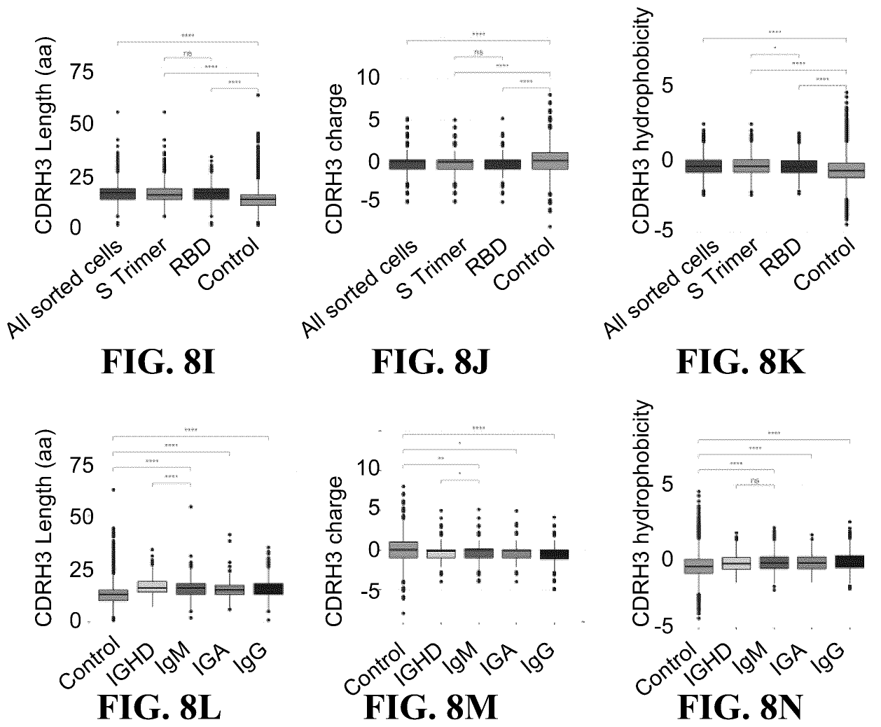

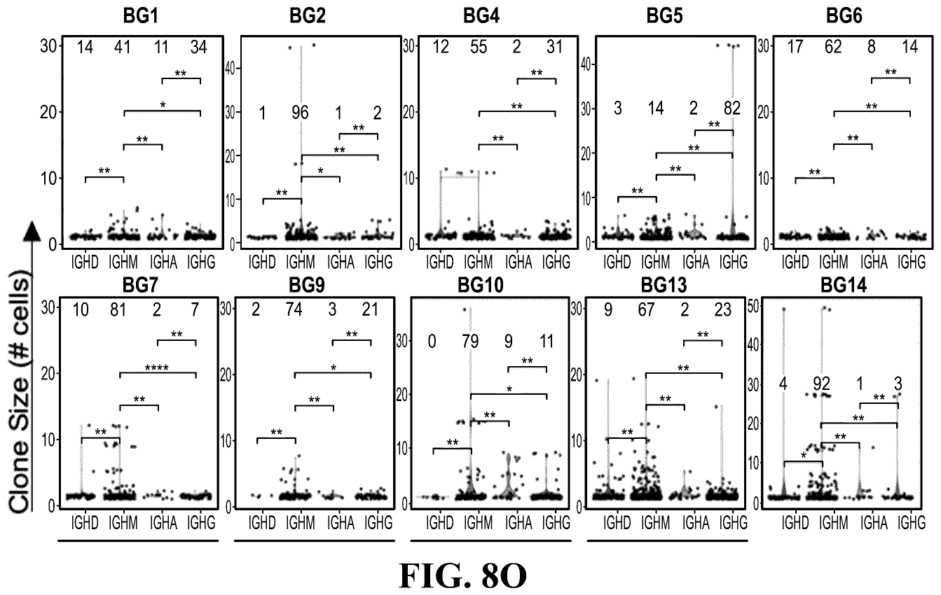

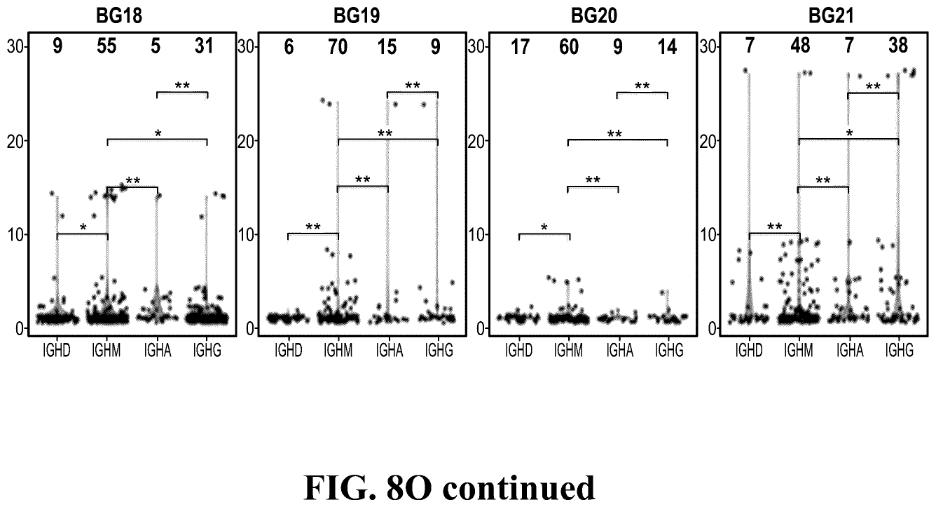

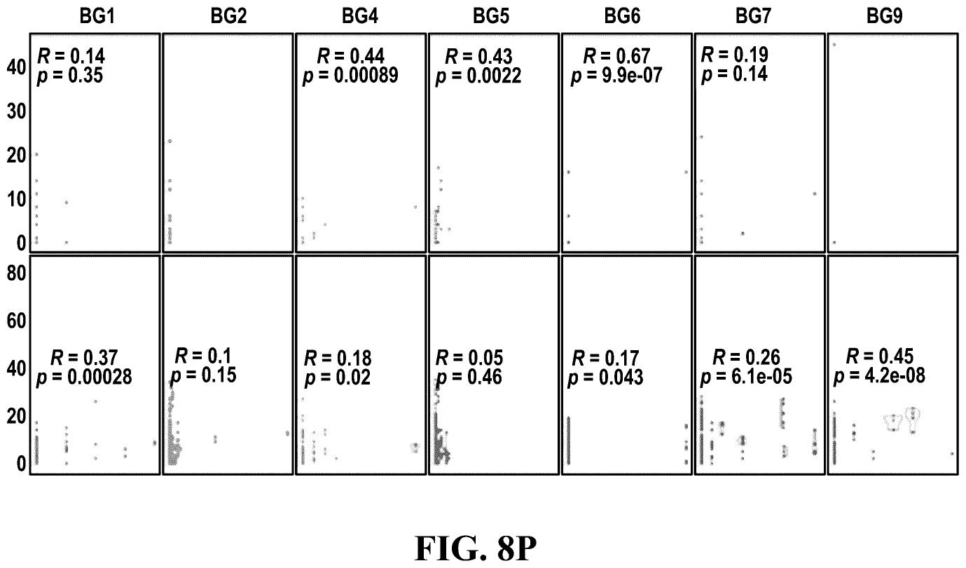

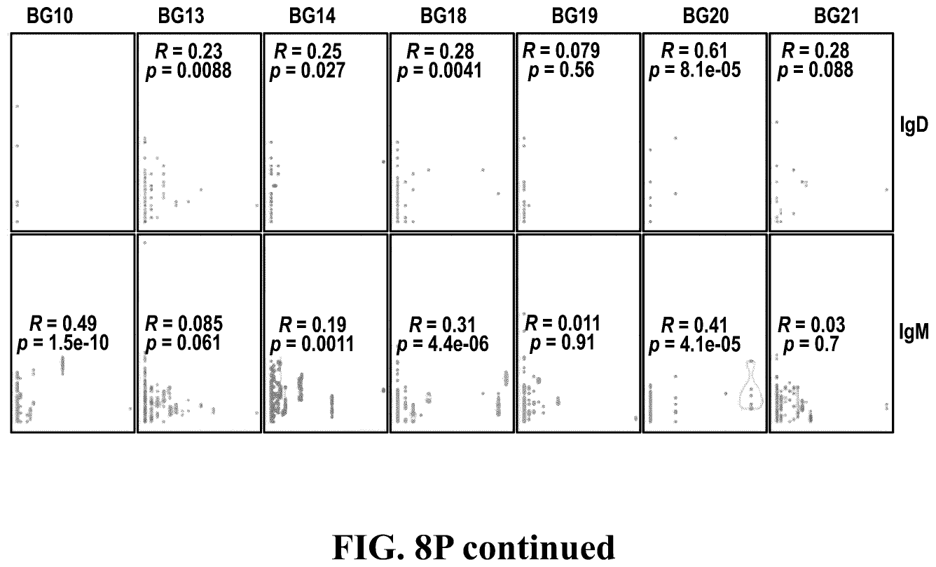

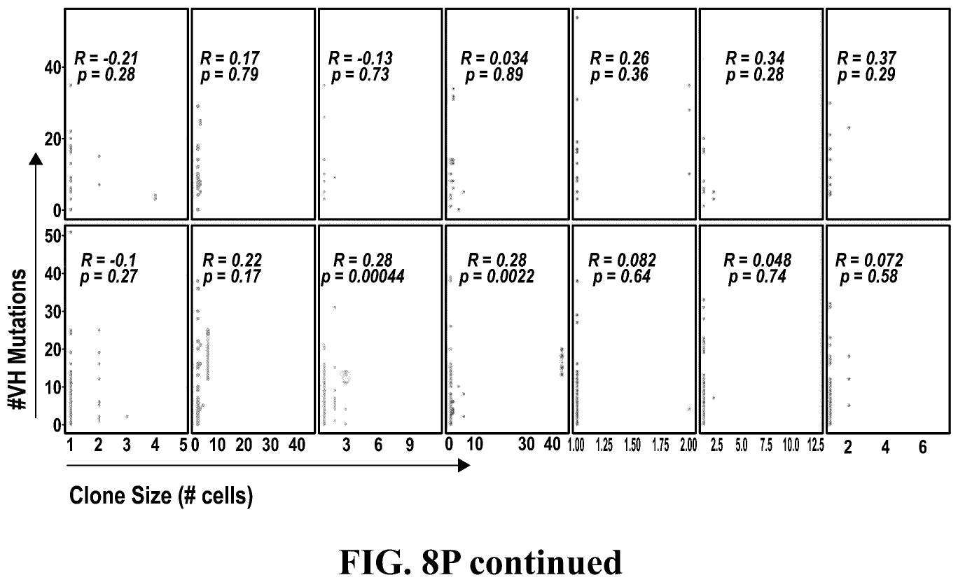

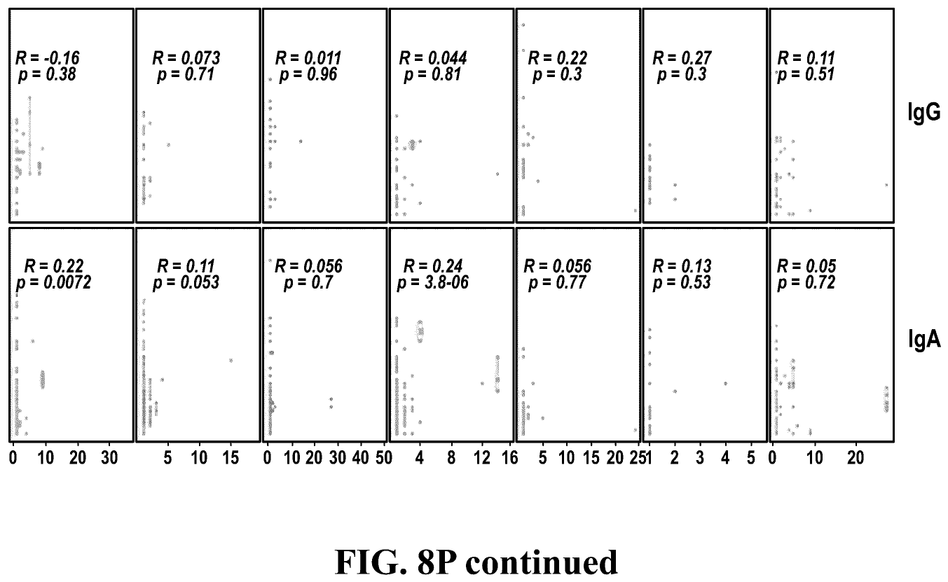

To evaluate serum neutralizing activity, Applicants used a pseudotyped virus with SARS-COV-2 spike (S) protein (Robbiani et al., 2020; Schmidt et al., 2020)(STAR Methods). Applicants detected serum neutralization in 11 of 14 (79%) subjects (FIG. 8A, Table 1). ID₅₀ titers ranged from 51 to 655, and no correlation was seen between serum neutralization and time since diagnosis or age of the subjects (FIG. 8B, C, Table 1). Although 3 of the top 4 neutralizing sera were obtained from male subjects, there was no statistically significant difference between serum neutralizing activity in female versus male subjects (FIG. 8D, Table 1). Applicants conclude that serum neutralizing activity was detected in a majority of subjects in this cohort of recently recovered non-hospitalized SARS-COV-2 patients.

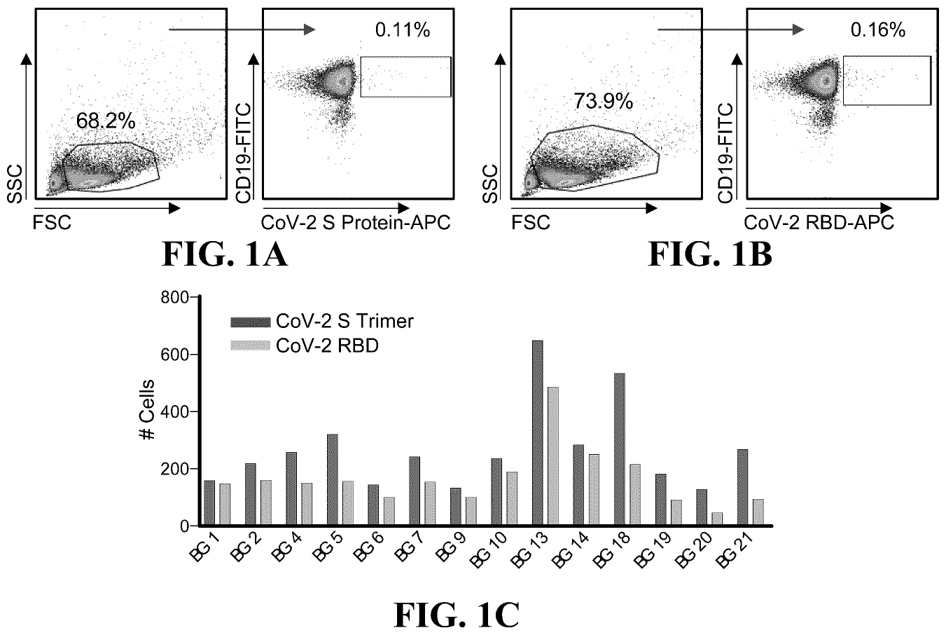

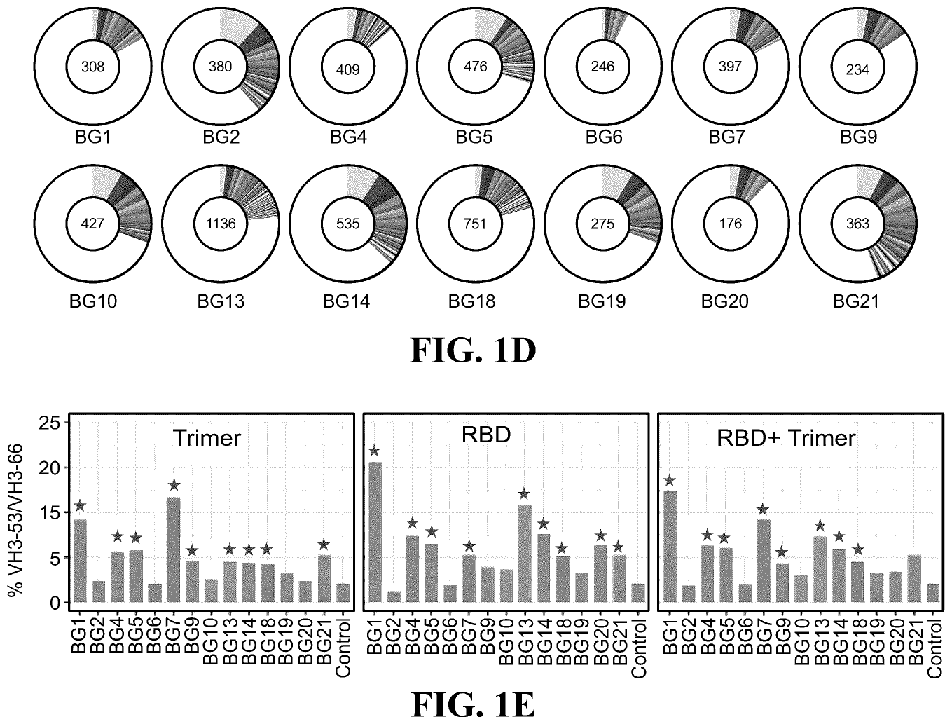

SARS-COV-2 binding B cell repertoires from recovered subjects are enriched for class 1 neutralizing antibodies against RBD. To characterize the B cell response against the SARS-COV-2 spike, Applicants sorted a total of 6,113 B cells from 14 subjects that bound to the SARS-COV-2 receptor binding domain (RBD) or SARS-COV-2 S trimer (Wrapp et al., 2020) using fluorescence-activated cell sorting (FACS) (FIGS. 1A, B). The frequency of SARS-COV-2 S- or RBD-binding B cells ranged from 0.05-0.3% of CD19+ B cells (FIGS. 1A, B). Applicants profiled sorted cells by 5′ directed scRNA-Seq for both mRNA and paired VDJ profiling, recovering matched single-cell VDJ and transcriptome profiles in 129-649 cells for SARS-COV-2 S and 47-487 cells for RBD per donor (FIG. 1C). Applicants identified clonally related B cells of each donor, by accounting for V and J gene similarities as well as sequence similarity between the heavy chain complementarity-determining region (CDRH3) amino acid sequences (Nouri and Kleinstein, 2018). Expanded B cell clones, which Applicants define as the clones with at least 2 clonally related cells in the samples, accounted for 7-45% of sorted B cells in the 14 subjects (FIG. 1D).

Applicants tested if the sorted B cell repertoires include cells that have undergone somatic hypermutation and class switch recombination, two steps in the pathway to generate high affinity B cell responses (Victora and Nussenzweig, 2012). Assessing immunoglobulin isotypes, Applicants found that the fractions of IgG⁺, IgM/IgD⁺ and IgA⁺ B cells varied substantially among subjects (9-48%, 48-87% and 3-12% respectively) (FIG. 8E). When Applicants investigated clonal expansion within each antibody isotype, Applicants found that in 8 of 14 subjects IgM+ clones were statistically significantly more expanded than either IgA+ clones or IgG+ clones or both (FIG. 8O). Inferring levels of somatic mutations (from RNA, Methods), the total number of inferred heavy chain somatic mutations was lower in SARS-COV-2 S- and RBD-binding B cells than in historic control memory B cells (Rubelt et al., 2012) (FIG. 8F). IgM⁺ and IgD⁺ SARS-CoV-2 binding B cells showed significantly lower levels of inferred heavy chain somatic mutations compared to IgG⁺ and IgA⁺ B cells (FIG. 8G). Lower levels of somatic mutations were especially observed in IgM+ B cells with low clonal expansion as 9/14 subjects showed a statistically significant positive correlation between clone size and number of inferred heavy chain mutations in their IgM+ repertoire as opposed to IgG+ and IgA+ cells where this positive correlation was only observed in 4 and 0 subjects respectively (FIG. 8P).

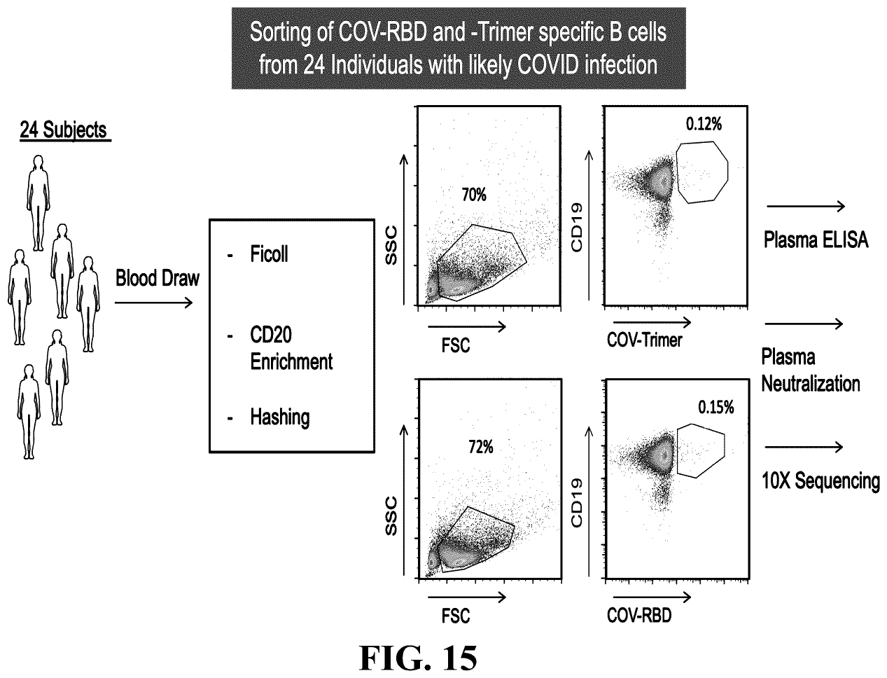

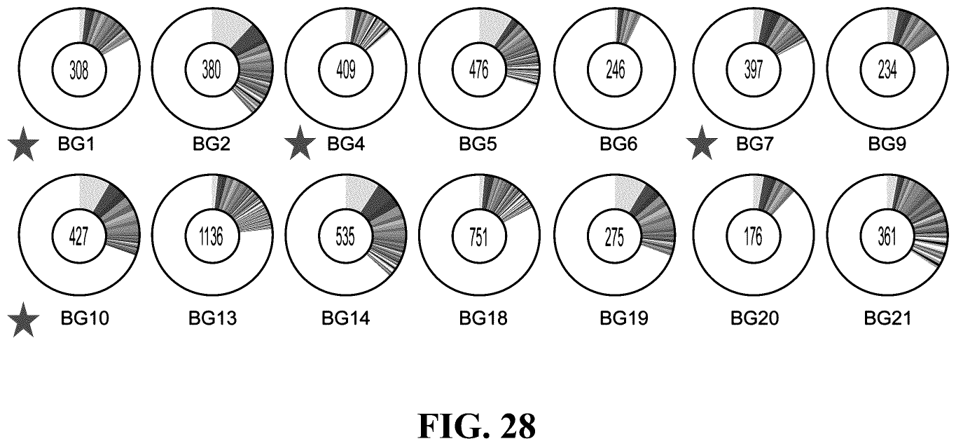

Applicants next tested if the sorted B cells were enriched for immunoglobulin genes VH3-53 and VH3-66, as class 1 antibodies that block ACE2 binding to the RBD show a preference for VH3-53 and VH3-66 heavy chain genes (Barnes et al., 2020b; Wu et al., 2020a; Yuan et al., 2020). Several class 1 antibodies have been isolated from different subjects, most of which carry CDRH3 regions that are shorter than 14 amino acids (IMGT definition; (Lefranc et al., 2015)) (Barnes et al., 2020b; Wu et al., 2020a; Yuan et al., 2020). Applicants thus compared the frequency of these two VH genes to combined historic control memory B cells from 14 healthy donors (Rubelt et al., 2012). 13 of the 14 recovered donors showed a higher fraction of combined VH3-53/VH3-66 in B cell repertoires against SARS-COV-2 S, RBD or both compared to historic memory B cell controls. This difference was statistically significant in 10 of 14 (71%, FDR<0.1, Two-proportions z-test) subjects (FIG. 1E) and remained significant in 10 of 14 (71%, FDR<0.1, Two-proportions z-test) subjects when considering only antibodies with CDRH3 regions shorter than 14 amino acids (not shown). Among other antibody features, the kappa and lambda light chain ratios were comparable to historic controls (FIG. 8H), whereas CDRH3 length and hydrophobicity were slightly increased and CDRH3 charge was decreased, and these differences existed for both S- and RBD-binding B cells (FIGS. 8I-K) and across Ig isotypes (FIGS. 8L-N). Thus, the B cell repertoire that binds to SARS-COV-2 S and SARS-COV-2 RBD in recovered individuals includes class switched IgG and IgA affinity maturated B cells and is enriched for heavy chain genes that are associated with class 1 neutralizing antibodies. See, also, FIGS. 15, 28 and 29.

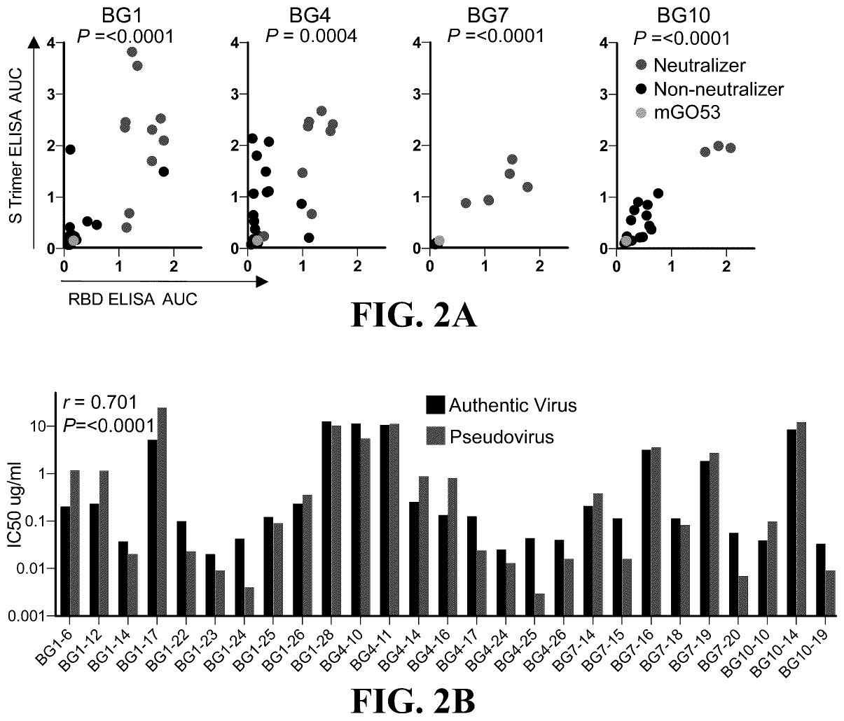

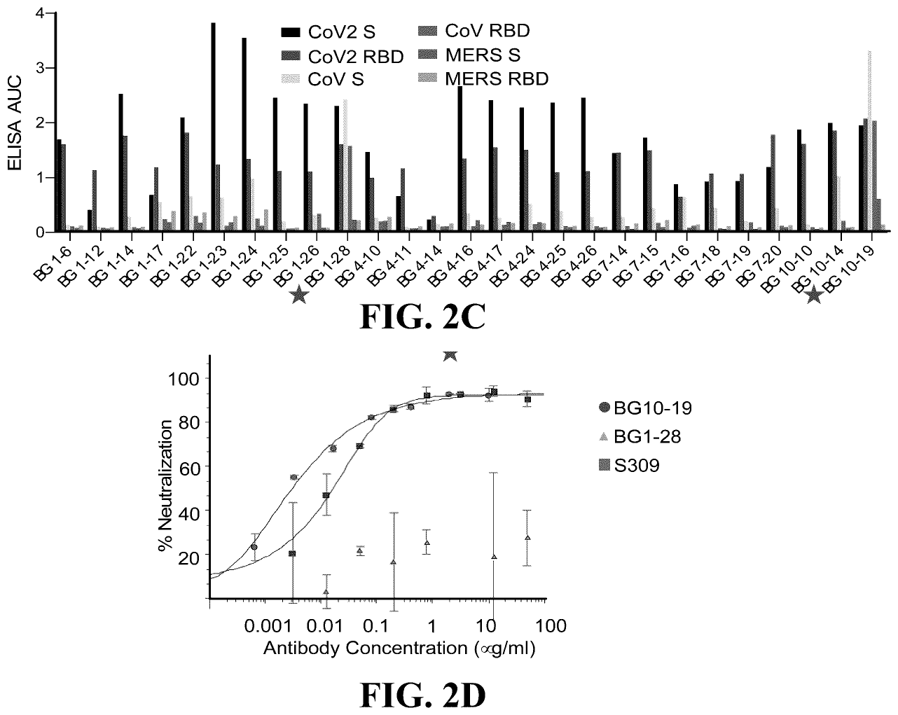

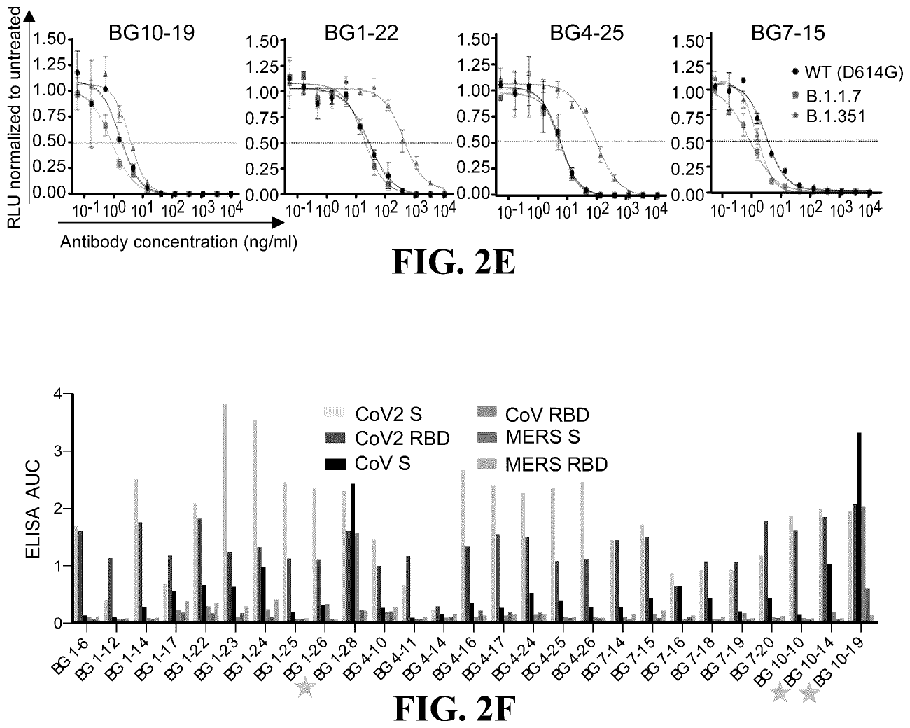

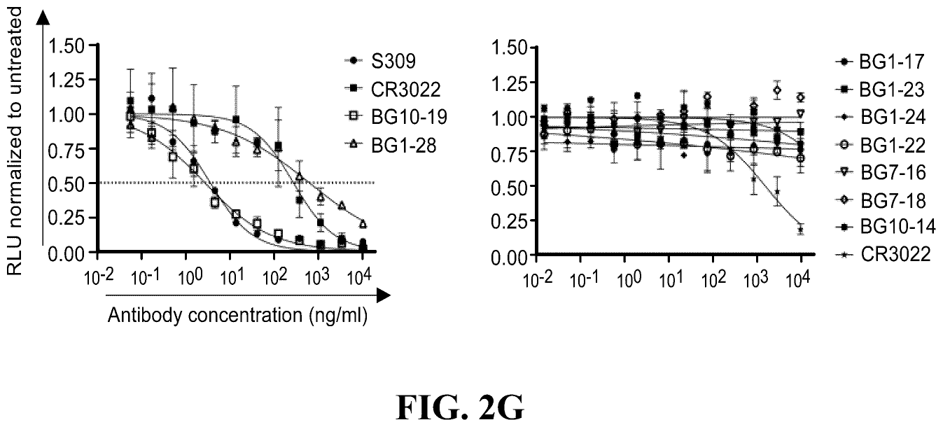

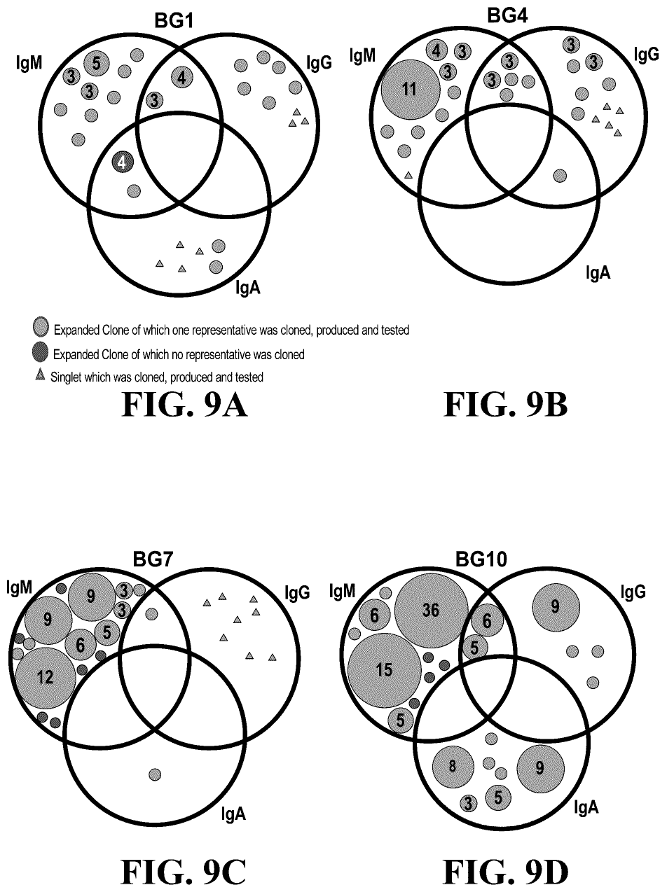

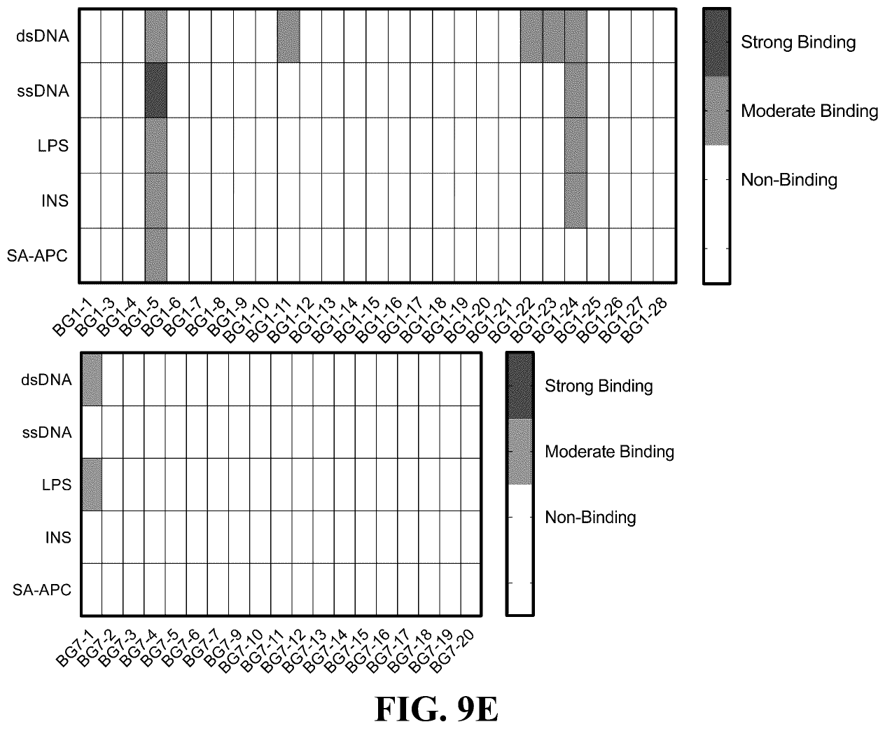

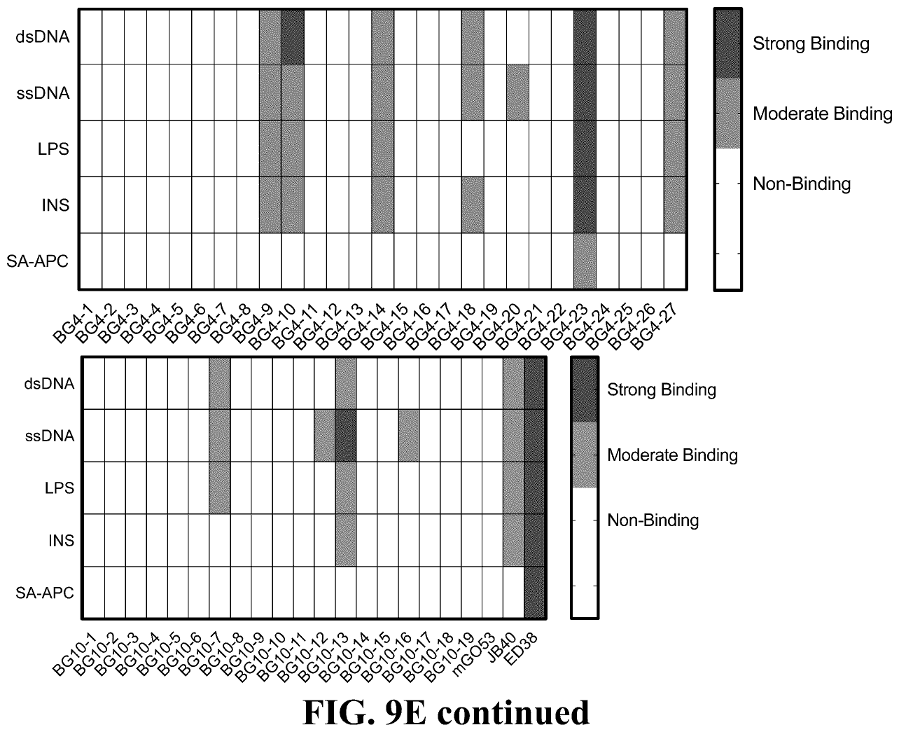

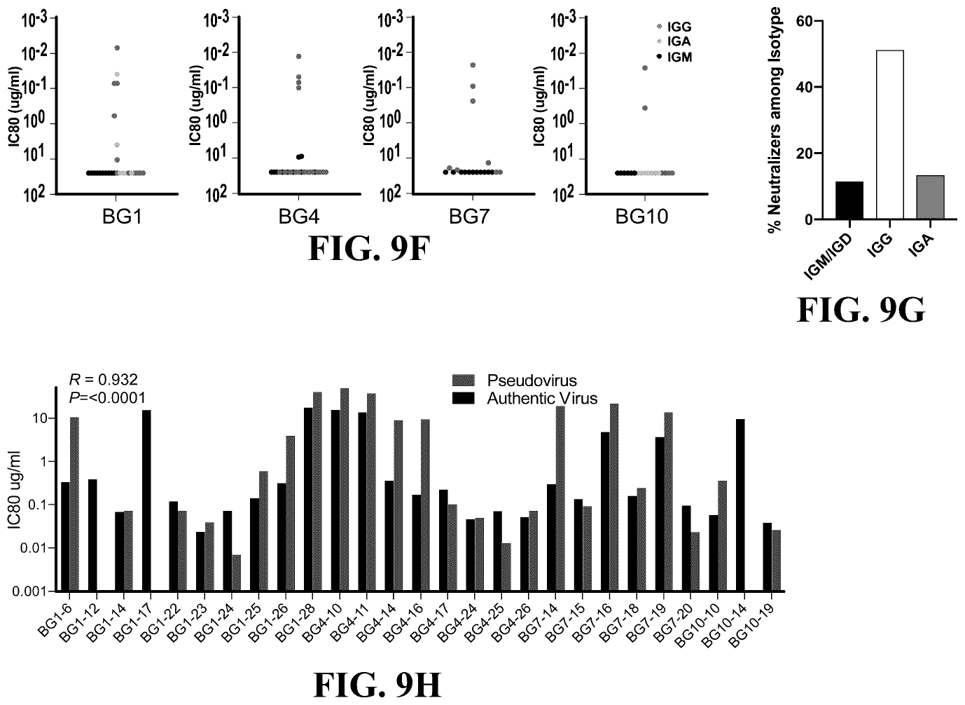

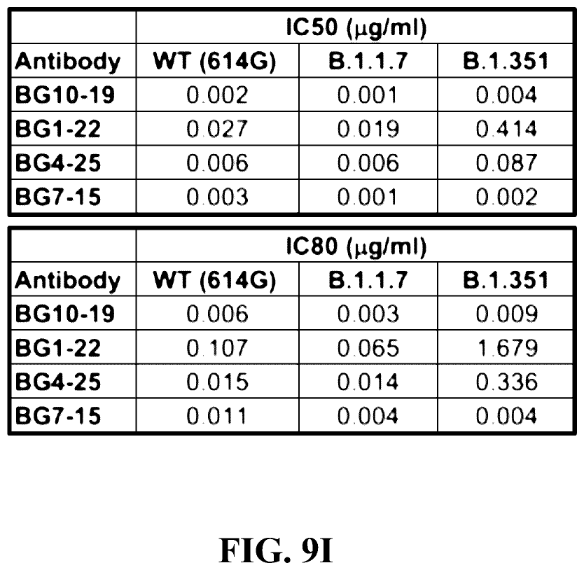

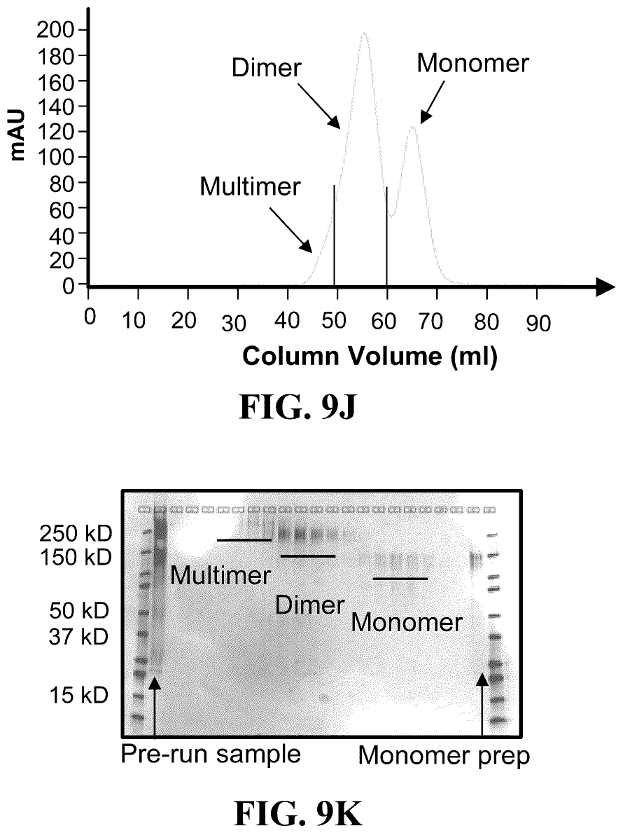

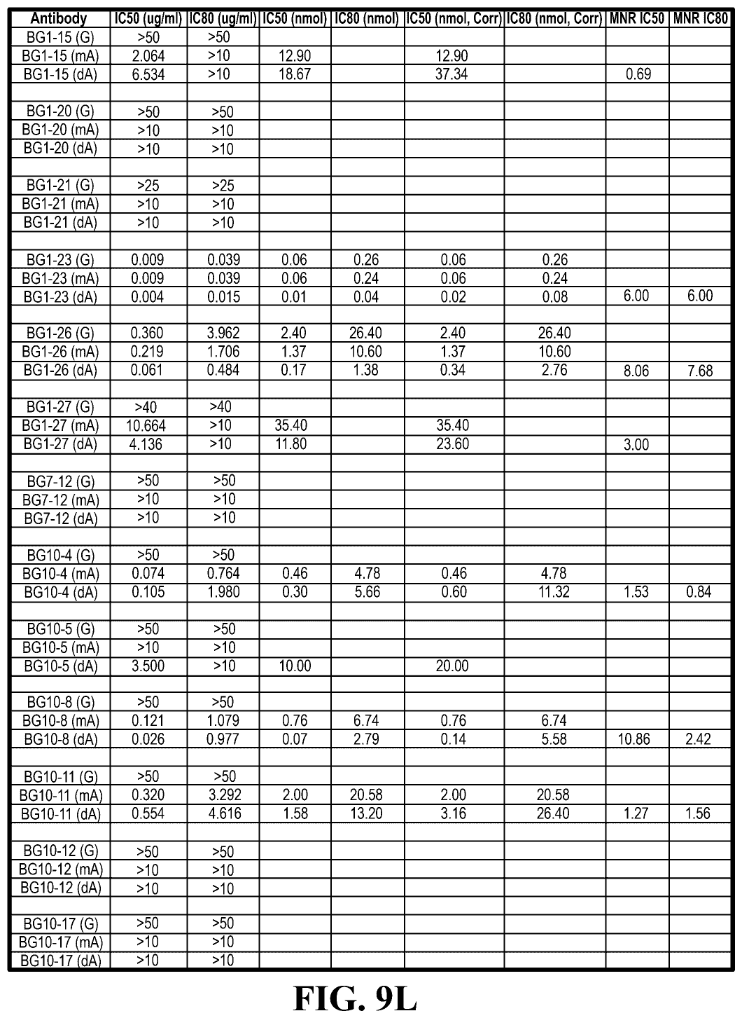

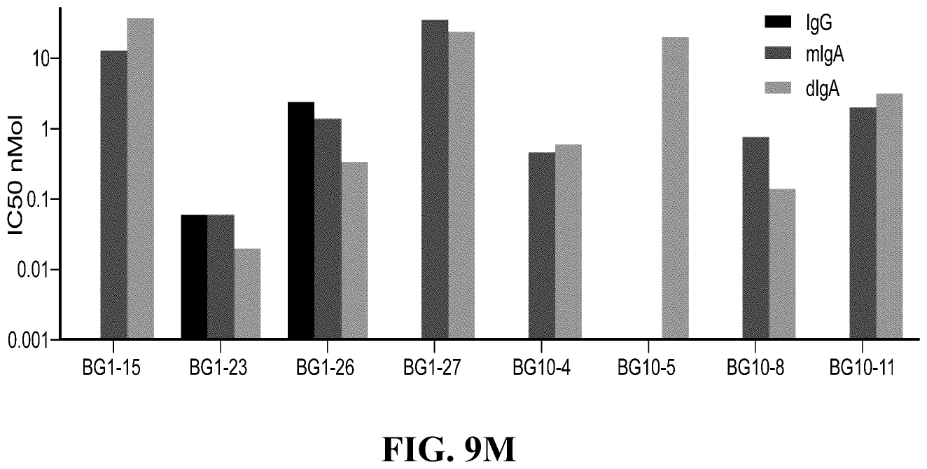

SARS-COV-2 spike binding monoclonal antibodies are directed against RBD and targets outside of the RBD. To functionally evaluate selected antibodies, Applicants chose 4 of the 14 subjects with the highest serum neutralization titers (FIG. 8A and Table 1) and produced a total of 92 antibodies from these donors (Table 2). The selection of antibodies consisted of 72 representatives from most of the expanded B cell clones in these subjects and 20 singlets (FIGS. 9A-D). All of the selected antibodies selected were expressed as IgG1s in order to allow for direct comparison of binding and neutralization and were initially evaluated for binding by ELISA. 56 of 92 antibodies (61%) showed strong or intermediate binding to either SARS-COV-2 S, RBD, or both in ELISA (FIG. 2A and Table 3). 42 of 56 strong or intermediate binders (75%) bound to both SARS-COV-2 S and RBD, 9 of 56 strong or intermediate binders (16%) only bound to SARS-CoV-2 S, and 5 of 56 (9%) only to RBD (FIG. 2A and Table 3). Applicants conclude that most antibodies isolated from SARS-COV-2 S- and RBD-binding B cells target either the RBD or epitopes on the spike outside of the RBD.

Table 2A-2B. Selected Antibodies for functional evaluation (related to FIGS. 2 and 9). All neutralization results in this table are from antibodies expressed as IgG1. A. Shown are isotype, VH gene, CDRH3 amino acid sequence, light chain kappa or lambda light chain use, V kappa/V lambda gene, light chain CDRL3 amino acid sequence, sorting bait used to identify the cell, antibody name, IC50 in μg/ml in SARS-COV-2 pseudovirus neutralization assay (PV), IC80 in μg/ml in SARS-COV-2 pseudovirus neutralization assay, IC50 in μg/ml in SARS-COV-2 authentic virus neutralization assay (LV), IC80 in μg/ml in SARS-COV-2 authentic virus neutralization assay for antibodies from subject BG1, BG4, BG7 and BG10 as indicated by the antibody names. (SEQ ID NOS: 1-184). B. Shown are the heavy chain sequences (HC), kappa light chain (KC) sequences and third lambda light chain (LC) sequences for the indicated antibodies tested (SEQ ID NOS: 185-368).

TABLE 2A

| | | | | | CDR3 | | | PV | PV | LV | LV |
|---|---|---|---|---|---|---|---|---|---|---|---|
| | VH | CDR3 | LIGHT | VK/VL | LIGHT | SORT | | IC50 | IG80 | IC50 | IG80 |
| ISOTYPE | GENE | HEAVY CHAIN | CHAIN | GENE | CHAIN | BAIT | Name | (ug/ml) | (ug/ml) | (ug/ml) | (ug/ml) |
| IGH | IGHV6- | CAKQWLANWFDPW | IGK | IGKV3- | CQQSESSPY | RBD | BG1- | >50 | >50 | | |
| M | 1 | | | 20 | TF | | 1 | | | | |
| IGHG | IGHV1- | CARGRAGYYDFWSTSS | IGL | IGLV1- | CAAWDDSL | RBD | BG1- | >50 | >50 | | |
| 1 | 8 | LDNGGMDVW | | 47 | SGVVF | | 3 | | | | |
| IGH | IGHV3- | CVKDVPSMYYIFWSGQ | IGK | IGKV1- | CQQYNTYS | RBD | BG1- | >50 | >50 | | |
| M | 23 | GTYW | | 5 | PITF | | 4 | | | | |
| IGH | IGHV3- | CAKDLPSRYYDFWSG | IGK | IGKV1- | CQQYNSYS | RBD | BG1- | >50 | >50 | | |
| M | 23 | QGNYW | | 5 | SITF | | 5 | | | | |
| IGHG | IGHV2- | CAREMATITGLDYW | IGK | IGKV1- | CQQSYSTP | RBD | BG1- | 1.178 | 10.581 | 0.2037 | 0.3314 |
| 1 | 70 | | | 39; | RTF | | 6 | | | | |
| | | | | IGK | | | | | | | |
| | | | | V1D-39 | | | | | | | |
| IGHA | IGHV2- | CARVPPTDYHFDSW | IGK | IGKV3- | CQHYGSSP | 2P | BG1- | >45 | >45 | | |
| 1 | 70 | | | 20 | PLTF | | 7 | | | | |
| IGH | IGHV1- | CARDTSGYRNPGYGM | IGK | IGKV3- | CQQRSNWP | 2P | BG1- | >50 | >50 | | |
| M | 18 | DVW | | 11 | PWTF | | 8 | | | | |
| IGHG | IGHV5- | CATGTFLAVPGFDFW | IGL | IGLV6-57; | CQSYDSGR | RBD | BG1- | >25 | >25 | | |
| 1 | 51 | | | IGLV6-57 | QGVF | | 9 | | | | |
| IGH | IGHV3- | CARDLQRYGSGSSLGP | IGK | IGKV1- | CQQLDTYP | RBD | BG1- | >50 | >50 | | |
| M | 48 | NYFDYW | | 9 | RF | | 10 | | | | |
| IGH | IGHV4- | CARRAQYYYDSSGYY | IGK | IGKV3- | CQQYNSYS | RBD | BG1- | >35 | >35 | | |
| M | 39 | DAFDIW | | 20 | TF | | 11 | | | | |
| IGHG | IGHV3- | CARDVFRYDSSGRPRY | IGK | IGKV2- | CMQALQTP | RBD | BG1- | 1.151 | >50 | 0.2338 | 0.3866 |
| 1 | 13 | AFDIW | | 28; | LTF | | 12 | | | | |
| | | | | IGKV2D- | | | | | | | |
| | | | | 28 | | | | | | | |
| IGHG | IGHV4- | CARETRWNWIDSW | IGK | IGKV4- | CQQYHNTP | 2P | BG1- | >30 | >30 | | |
| 1 | 59; | | | 1 | WTF | | 13 | | | | |
| | IGHV4- | | | | | | | | | | |
| | 59 | | | | | | | | | | |
| IGHG | IGHV3- | CARDLVDYGMDVW | IGK | IGKV1- | CQQLNSYP | RBD | BG1- | 0.020 | 0.072 | 0.03716 | 0.06751 |
| 1 | 53 | | | 9 | PYTF | | 14 | | | | |
| IGHA | IGHV3- | CAKDGRRDGYNHALD | IGK | IGKV1- | CQQYSSYP | RBD | BG1- | >50 | >50 | | |
| 1 | 43D | NW | | 5 | TF | | 15 | | | | |
| IGH | IGHV3- | CAKGRESTSIGGWFDP | IGK | IGKV1- | CQHYNAYP | 2P | BG1- | >37 | >37 | | |
| M | 30; | W | | 16 | DHL | | 16 | | | | |
| | IGHV3- | | | | | | | | | | |
| | 30-5 | | | | | | | | | | |
| IGH | IGHV | CARTIVGGTNWYFDL | IGL | IGLV1- | CASWDDSV | RBD | BG1- | 24.883 | >50 | 5.174 | 15.25 |
| M | 2-70 | W | | 44 | NGVF | | 17 | | | | |
| IGH | IGHV | CARRVMGSGWLDSW | IGL | IGLV2- | CSSYTSSST | 2P | BG1- | >50 | >50 | | |
| M | 4-39 | | | 14 | WVF | | 18 | | | | |
| IGHG | IGHV | CAKDMGNGFYYYYGL | IGK | IGKV2D- | CMQTIHLP | RBD | BG1- | >30 | >30 | | |
| 1 | 3-9 | DVW | | 29 | RVTF | | 19 | | | | |
| IGHA | IGHV3- | CAKDAPDVVLVPVPY | IGK | IGKV3- | CQHYGSSR | 2P | BG1- | >50 | >50 | | |
| 2 | 23; | YHYGLEVW | | 20 | RTF | | 20 | | | | |
| | IGHV3- | | | | | | | | | | |
| | 23D | | | | | | | | | | |
| IGHA | IGHV3- | CARERSGSYYARVELG | IGL | IGLV2- | CSSYTSSIT | RB | BG1- | >25 | >25 | | |
| 1 | 30 | YW | | 14 | GVF | D | 21 | | | | |
| IGHG | IGHV3- | CASSRPPIGQLVPGLDL | IGL | IGLV1- | CQSYDSRL | RBD | BG1- | 0.023 | 0.072 | 0.09962 | 0.1181 |
| 1 | 66; | DWFDPW | | 40 | SGWVF | | 22 | | | | |
| | IGHV3- | | | | | | | | | | |
| | 66 | | | | | | | | | | |
| IGHA | IGHV | CARDSYRGGGIW | IGK | IGKV1- | CQQYDNLP | RBD | BG1- | 0.009 | 0.039 | 0.02004 | 0.02348 |
| 1 | 3-53 | | | 33; | QTF | | 23 | | | | |
| | | | | IGKV1D- | | | | | | | |
| | | | | 33 | | | | | | | |
| IGHG | IGHV1- | CARGPYYYDSGGYYL | IGK | IGKV3- | CQQYAGSP | RBD | BG1- | 0.004 | 0.007 | 0.04294 | 0.07228 |
| 1 | 69; | DYW | | 20 | RTF | | 24 | | | | |
| | IGHV1- | | | | | | | | | | |
| | 69 | | | | | | | | | | |
| IGHG | IGHV3- | CARDMGGDVFDIW | IGK | IGKV1- | CQQLNSSPP | RBD | BG1- | 0.091 | 0.59 | 0.1224 | 0.14601 |
| 1 | 53 | | | 9 | KLTF | | 25 | | | | |
| IGHA | IGHV4- | CAAHITVFGIITGNWFD | IGL | IGLV1- | CQSYASSLS | RBD | BG1- | 0.360 | 3.962 | 0.235 | 0.3127 |
| 1 | 39 | PW | | 40 | VHVVF | | 26 | | | | |
| IGHA | IGHV3- | CARESAAAWGWLDPW | IGK | IGKV139; | CQQSYSMP | RBD | BG1- | >40 | >40 | | |
| 1 | 53 | | | IGKV1D- | YTF | | 27 | | | | |
| | | | | 39 | | | | | | | |
| IGHG | IGHV3- | CAKDLGYSYGSEIFDY | IGK | IGKV1- | CQQSYSTP | RBD | BG1- | 10.398 | 39.842 | 12.75 | 17.54 |
| 1 | 23 | W | | 39; | VF | | 28 | | | | |
| | | | | IGKV1D- | | | | | | | |
| | | | | 39 | | | | | | | |
| IGH | IGHV3- | CARARSGSYYSAIDYW | IGK | IGKV1- | CQQYDYLP | 2P | BG4- | >50 | >50 | | |
| M | 30-3 | | | 33; | LTF | | 1 | | | | |
| | | | | IGKV1D- | | | | | | | |
| | | | | 33 | | | | | | | |
| IGH | IGHV3- | CAKEAYFTAWLHFPFD | IGK | IGKV3- | CQQYGSSL | 2P | BG4- | >50 | >50 | | |
| M | 23; | PW | | 20 | TF | | 2 | | | | |
| | IGHV3- | | | | | | | | | | |
| | 23D | | | | | | | | | | |
| IGHG | IGHV3- | CARDRDDYGDPYGMD | IGL | IGLV3- | CYSVADNM | 2P | BG4- | >50 | >50 | | |
| 3 | 30; | VW | | 27 | GVF | | 3 | | | | |
| | IGHV3- | | | | | | | | | | |
| | 30-3 | | | | | | | | | | |
| IGHG | IGHV3- | CTTGPTVRGVSVYW | IGL | IGLV7- | CLLSYSGA | 2P | BG4- | >50 | >50 | | |
| 2 | 15 | | | 46 | WVF | | 4 | | | | |
| IGHG | IGHV1- | CARSVGYDAFDVW | IGL | IGLV1- | CAAWDDSL | RBD | BG4- | >45 | >45 | | |
| 1 | 2 | | | 47 | FVVF | | 5 | | | | |
| IGH | IGHV3- | CARDLMDYDILTGYYP | IGK | IGKV4- | CQQYFSTPP | RBD | BG4- | >50 | >50 | | |
| M | 21 | AGYW | | 1 | STF | | 6 | | | | |
| IGH | IGHV1- | CARGPPPPAITMIVVTT | IGK | IGKV22 | CMQALQTP | RBD | BG4- | >35 | >35 | | |
| M | 2 | VWTS | | 28; | YTF | | 7 | | | | |
| | | | | IGKV2D- | | | | | | | |
| | | | | 28 | | | | | | | |
| IGHG | IGHV5- | CARLETTVTTDWFDP | IGL | IGLV2- | CSSYTRGN | RBD | BG4- | >50 | >50 | | |
| 1 | 10-1 | W | | 14 | YVF | | 8 | | | | |
| IGHG | IGHV3- | CTIGNWHGSGNNIMHD | IGK | IGKV4- | CHQYYLVP | RBD | BG4- | >50 | >50 | | |
| 3 | 49 | YW | | 1 | FTF | | 9 | | | | |
| IGHG | IGHV1- | CARDRWFGKLEGSFD | IGL | IGLV2- | CCSYATGS | RBD | BG4- | 5.53 | 48.924 | 11.48 | 15.58 |
| 1 | 2 | VW | | 23; | SVVF | | 10 | | | | |
| | | | | IGLV2- | | | | | | | |
| | | | | 23; | | | | | | | |
| | | | | IGLV2- | | | | | | | |
| | | | | 23 | | | | | | | |
| IGH | IGHV4- | CARDTVVVVGAHAFDI | IGK | IGKV4- | CQQYYTTP | RBD | BG4- | 11.335 | 36.996 | 10.7 | 13.55 |
| M | 34 | W | | 1 | LTF | | 11 | | | | |
| IGHG | IGHV4- | CARPQVLYYYGSGSYS | IGL | IGLV1- | CQSYDSSLS | 2P | BG4- | >50 | >50 | | |
| 1 | 39 | HWFDPW | | 40 | AVWVF | | 12 | | | | |
| IGH | IGHV3- | CARGSGWYVGFDYW | IGL | IGLV1- | CAAWDDSL | RBD | BG4- | >50 | >50 | | |
| M | 48 | | | 47 | SALVF | | 13 | | | | |
| IGH | IGHV1- | CARVTTIPQYDYGDYY | IGL | IGLV2- | CCSYAGSS | 2P | BG4- | 0.878 | 8.971 | 0.2553 | 0.3563 |
| M | 2 | YYYGMDVW | | 23 | TLVF | | 14 | | | | |
| IGHG | IGHV4- | CARDLGQDW | IGK | IGKV3- | CQQYGSSP | 2P | BG4- | >50 | >50 | | |
| 3 | 59 | | | 20 | WTF | | 15 | | | | |
| IGH | IGHV4- | CARDIFGSGSPPSGVAF | IGL | IGLV2- | CSSHTSSSS | RBD | BG4- | 0.806 | 9.388 | 0.1342 | 0.1703 |
| M | 61 | DYW | | 14 | PHVVF | | 16 | | | | |
| IGHG | IGHV3- | CARESYGMDVW | IGK | IGKV3- | CQQYGSSP | 2P | BG4- | 0.024 | 0.102 | 0.1276 | 0.2202 |
| 1 | 53 | | | 20 | GTF | | 17 | | | | |
| IGHG | IGHV1- | CARVKEWGSYYSDSG | IGL | IGLV2- | CCSYAGTY | 2P | BG4- | >50 | >50 | | |
| 1 | 69; | SHGKNYFDYW | | 11 | TVIF | | 18 | | | | |
| | IGHV1- | | | | | | | | | | |
| | 69D | | | | | | | | | | |
| IGH | IGHV1- | CARDGYSSSFIPGPRNY | IGK | IGKV3- | CQQFNNWP | RBD | BG4- | >50 | >50 | | |
| M | 2 | YGMDVW | | 15 | RGTF | | 19 | | | | |
| IGHG | IGHV1- | CARPRGRLITFGGLIVT | IGK | IGKV2- | CMQALQTP | 2P | BG4- | >50 | >50 | | |
| 1 | 18 | TLDYYYGMGVW | | 28; | VTF | | 20 | | | | |
| | | | | IGKV2D- | | | | | | | |
| | | | | 28 | | | | | | | |
| IGHG | IGHV1- | CATDKADTSWWEFDP | IGL | IGLV7- | CLLSYSGV | RBD | BG4- | >35 | >35 | | |
| 1 | 2 | W | | 46 | VVF | | 21 | | | | |
| IGHG | IGHV3- | CARFSKLYQWSFDIW | IGL | IGLV2- | CCSYAGTY | 2P | BG4- | >50 | >50 | | |
| 1 | 53 | | | 11 | TPVVF | | 22 | | | | |
| IGH | IGHV3- | CARGTHYGSGSYYHW | IGK | IGKV3- | CQQYNNW | RBD | BG4- | >50 | >50 | | |
| M | 53 | | | 15 | PPGLKITF | | 23 | | | | |
| IGHG | IGHV3- | CVRDLDYYGMDVW | IGK | IGKV1- | CQQLNSSP | RBD | BG4- | 0.013 | 0.05 | 0.02494 | 0.04633 |
| 1 | 53 | | | 9 | GSLTF | | 24 | | | | |
| IGHG | IGHV3- | CARDLEVVGAMDVW | IGK | IGKV3- | CQQYGSPM | 2P | BG4- | 0.003 | 0.013 | 0.04379 | 0.0706 |
| 1 | 53 | | | 20 | YTF | | 25 | | | | |
| IGHG | IGHV3- | CAREGPHGMDVW | IGK | IGKV1- | CQQYDNLP | RBD | BG4- | 0.016 | 0.072 | 0.04001 | 0.05179 |
| 1 | 53 | | | 33; | PTF | | 26 | | | | |
| | | | | IGK | | | | | | | |
| | | | | V1D-33 | | | | | | | |
| IGHG | IGHV3- | CARGPVRGVITHFDYW | IGL | IGLV2- | CSSYTSSST | 2P | BG4- | >50 | >50 | | |
| 3 | 53 | | | 14 | SSVF | | 27 | | | | |
| IGHD | IGHV3- | CVKSSSGWYLRLGYW | IGK | IGKV2- | CMQALQTP | 2P | BG7- | >50 | >50 | | |
| | 30; | | | 28; | TF | | 1 | | | | |
| | IGHV3- | | | IGKV2D- | | | | | | | |
| | 30-5 | | | 28 | | | | | | | |
| IGH | IGHV3- | CAREGRRGQWSWFDP | IGK | IGKV3- | CQQFGSSPL | 2P | BG7- | >50 | >50 | | |
| M | 53 | W | | 20 | YTF | | 2 | | | | |
| IGH | IGHV3- | CAGSPVRGVITHFDYW | IGL | IGLV2- | CSSYTSSST | 2P | BG7- | >50 | >50 | | |
| M | 53 | | | 14 | QVF | | 3 | | | | |
| IGH | IGHV4- | CARVDRRYCIITSCYIF | IGK | IGKV4- | CQQYYSTPI | RBD | BG7- | >50 | >50 | | |
| M | 61 | DYW | | 1 | TF | | 4 | | | | |
| IGH | IGHV3- | CARGRDGYYYGMDV | IGK | IGKV1- | CQQLNSLF | 2P | BG7- | >50 | >50 | | |
| M | 30-3 | W | | 9 | TF | | 5 | | | | |
| IGH | IGHV1- | CARAPPFSSSSIGVDYW | IGK | IGKV1- | CQQYNSYP | 2P | BG7- | >50 | >50 | | |
| M | 2 | | | 16 | LTF | | 6 | | | | |
| IGH | IGHV1- | CAKDHCATTNCFEYLY | IGK | IGKV2- | CMQSLQTR | 2P | BG7- | >50 | >50 | | |
| M | 2 | FGMGVW | | 28; | TF | | 7 | | | | |
| | | | | IGKV2D- | | | | | | | |
| | | | | 28 | | | | | | | |
| IGH | IGHV4- | CARDLTYYGSNGYTN | IGK | IGKV1- | CQQYNTYF | 2P | BG7- | >50 | >50 | | |
| M | 4 | W | | 5 | WTF | | 9 | | | | |
| IGH | IGHV3- | CASSPVRGVITQFDYW | IGL | IGLV2- | CSSYTSRSP | 2P | BG7- | >50 | >50 | | |
| M | 53 | | | 14 | WVF | | 10 | | | | |
| IGHG | IGHV5- | CARHDGSKVTTNDYW | IGK | IGKV3- | CQQRRDWP | 2P | BG7- | >50 | >50 | | |
| 1 | 51 | | | 11 | GTF | | 11 | | | | |
| IGHA | IGHV3- | CARDRSGSYYRPGYW | IGK | IGKV1-33; | CQQYGNLP | 2P | BG7- | >50 | >50 | | |
| 1 | 30-3 | | | IGKV1D- | PLTF | | 12 | | | | |
| | | | | 33 | | | | | | | |
| IGH | IGHV3- | CAQTGGSYFGPFDYW | IGL | IGLV2- | CYSYTTSST | 2P | BG7- | >50 | >50 | | |
| M | 30; | | | 14 | PYVF | | 13 | | | | |
| | IGHV3- | | | | | | | | | | |
| | 30; | | | | | | | | | | |
| | IGHV3- | | | | | | | | | | |
| | 30-5 | | | | | | | | | | |
| IGHG | IGHV3- | CAKDILYGDYVGGFAY | IGL | IGLV2- | CSSYTTSST | RBD | BG7- | 0.385 | 19.178 | 0.2084 | 0.2981 |
| 1 | 9 | W | | 14 | LVF | | 14 | | | | |
| IGHG | IGHV1- | CAIPYSSVTFDCW | IGK | IGKV3- | CQQYGSSR | RBD | BG7- | 0.016 | 0.092 | 0.1139 | 0.1345 |
| 1 | 18 | | | 20 | PTF | | 15 | | | | |
| IGHG | IGHV3- | CARDSQPHYYYGVDV | IGL | IGLV2- | CSSYTKTRT | RBD | BG7- | 3.5992 | 21.792 | 3.172 | 4.817 |
| 1 | 53 | W | | 14 | LYVF | | 16 | | | | |
| IGHG | IGHV5- | CAKKKDDRAEAVFDY | IGL | IGLV1- | CATWDDSL | RBD | BG7- | >50 | >50 | | |
| 1 | 51 | W | | 44 | NTYVF | | 17 | | | | |
| IGHG | IGHV3- | CARGAAMVKELFDYW | IGK | IGKV4- | CQQYYSTP | RBD | BG7- | 0.083 | 0.244 | 0.1152 | 0.1601 |
| 1 | 53 | | | 1 | LTF | | 18 | | | | |
| IGHG | IGHV3- | CARVRWLQGGIDYW | IGL | IGLV6- | CQSYDSGS | RBD | BG7- | 2.735 | 13.72 | 1.854 | 3.653 |
| 1 | 7 | | | 57 | WVF | | 19 | | | | |
| IGHG | IGHV1- | CARGGRYCSSTTCYSG | IGL | IGLV1- | CQSYDSSLS | RBD | BG7- | 0.007 | 0.023 | 0.05679 | 0.0942 |
| 1 | 8 | VGMDVW | | 40 | GWVF | | 20 | | | | |
| IGH | IGHV3- | CAKSNSSGLTPFDHW | IGK | IGKV1- | CQQSYSNP | RBD | BG10- | >50 | >50 | | |
| M | 9 | | | 39; | FTF | | 1 | | | | |
| | | | | IGKV1D- | | | | | | | |
| | | | | 39 | | | | | | | |
| IGH | IGHV4- | CARRDGSYFDYW | IGL | IGLV3- | CQAGDSDT | RBD | BG10- | >50 | >50 | | |
| M | 39 | | | 1 | VLF | | 2 | | | | |
| IGHG | IGHV4- | CARHVSDGQSIGWLYY | IGL | IGLV3- | CQVWDRSS | RBD | BG10- | >50 | >50 | | |
| 1 | 39 | FDCW | | 21 | VF | | 3 | | | | |
| IGHA | IGHV4- | CARLAPFGERRVDQYY | IGK | IGKV2D- | CMQSTQLP | RBD | BG10- | >50 | >50 | | |
| 1 | 31 | LMDVW | | 29 | WTF | | 4 | | | | |
| IGHA | IGHV4- | CARAYYYYYMDVW | IGL | IGLV1- | CSAWDDSL | RBD | BG10- | >50 | >50 | | |
| 1 | 4 | | | 47 | RGPVF | | 5 | | | | |
| IGH | IGHV4- | CARADRGYCTITDCFIF | IGK | IGKV4- | CQQYYSTP | RBD | BG10- | >50 | >50 | | |
| M | 61 | NYW | | 1 | LTF | | 6 | | | | |
| IGHG | IGHV3- | CTRGAHYDFWRGYYT | IGL | IGLV1- | CQSYDSRL | RBD | BG10- | >50 | >50 | | |
| 1 | 13 | DDHHYMDVW | | 40 | SAWVF | | 7 | | | | |
| IGHA | IGHV3- | CVRGEGGPHDAFDIW | IGL | IGLV3- | CQVWDSSR | RBD | BG10- | >50 | >50 | | |
| 1 | 11 | | | 21 | DLRVF | | 8 | | | | |
| IGH | IGHV3- | CVRCGYDDWYFDVW | IGL | IGLV3- | CQAWDSST | 2P | BG10- | >50 | >50 | | |
| M | 7 | | | 1 | GVF | | 9 | | | | |
| IGHG | IGHV1- | CGRGPTTAKSGVEWFD | IGL | IGLV1- | CQSFDSSLS | RBD | BG10- | 0.099 | 0.359 | 0.03891 | 0.05747 |
| 1 | 8 | PW | | 40 | GFYVF | | 10 | | | | |
| IGHA | IGHV3- | CAKDMGWNVFQGLD | IGL | IGLV2- | CTSYRSSSL | RBD | BG10- | >50 | >50 | | |
| 1 | 9 | VW | | 14 | QGVF | | 11 | | | | |
| IGHA | IGHV1- | CARSWSAYYNWFDPW | IGK | IGKV3- | CQHYGSSP | 2P | BG10- | >50 | >50 | | |
| 1 | 18 | | | 20 | PVTF | | 12 | | | | |
| IGH | IGHV3- | CTTDRHVRGTYWGAY | IGL | IGLV1- | CAAWDDSL | RBD | BG10- | >50 | >50 | | |
| M | 15; | YYYGMDVW | | 44 | NGLYVF | | 13 | | | | |
| | IGHV3- | | | | | | | | | | |
| | 15 | | | | | | | | | | |
| IGHG | IGHV3- | CARAVRISSGYGLYGD | IGL | IGLV3- | CQVWDSSS | 2P | BG10- | 12.318 | >50 | 8.683 | 9.542 |
| 1 | 74 | SDYFDYW | | 21 | DPWVF | | 14 | | | | |
| IGH | IGHV3- | CARARGDAFDIW | IGL | IGLV1- | CGTWDNSL | RBD | BG10- | >50 | >50 | | |
| M | 48 | | | 51 | SVGVF | | 15 | | | | |
| IGHG | IGHV5- | CARPLFYCSGGSCLTG | IGL | IGLV2- | CSSYAGSD | 2P | BG10- | >50 | >50 | | |
| 1 | 51 | YW | | 8 | NWVF | | 16 | | | | |
| IGHA | IGHV3- | CARGRRDGNDLAPDY | IGL | IGLV3- | CYSTDSSD | 2P | BG10- | >50 | >50 | | |
| 1 | 74; | W | | 10 | NHRVF | | 17 | | | | |
| | IGHV3- | | | | | | | | | | |
| | 74 | | | | | | | | | | |
| IGHA | IGHV3- | CVKDLGAYASGRLAD | IGK | IGKV1- | CQQSYTTP | RBD | BG10- | >50 | >50 | | |
| 1 | 30; | W | | 39; | YTF | | 18 | | | | |
| | IGHV3- | | | IGK39 | | | | | | | |
| | 30-5 | | | | | | | | | | |
| IGHG | IGHV5- | CARTQWGYNYGSHFF | IGL | IGLV1- | CAAWDASL | RBD | BG10- | 0.009 | 0.026 | 0.03334 | 0.03846 |
| 1 | 51 | YMDVW | | 47 | SGYVF | | 19 | | | | |

TABLE 2B
Amino acid sequences of the variable regions of the antibodies produced and tested
(heavy chain sequences (HC), kappa light chain (KC) sequences and lambda light chain (LC)
sequences). (SEQ ID NOS: 185-368).

| | |
|---|---|
| HCBG1-1 | TGVHSQVQLQQSGPGLVKPSQTLSLTCAISGDSVSSKNAAWNWIRQSPSRGLEWLGTTYYRSKWYN |
| | DYAVSVKGRIAINPDTSKNQFSLQLHSVTPEDTAVYYCAKQWLANWFDPWGQGTQVTVSSAST |
| HCBG1-3 | TGVHSQVQLVQSGAEVKKPGASVKVSCKASGYTFTSYDINWVRQATGQGLEWMGWMNPNSGNTG |
| | YAQKFQGRVTMTRNTSISTAYMELSSLTSEDTAVYYCARGRAGYYDFWSTSSLDNGGMDVWGQGT |
| | TVTVSSAST |
| HCBG1-4 | TGVHSEVHLVESGGDLVHPGGSLRLSCVASGFTFTDYAMTWVRQAPGKGLEWVSTISGNGGTTNTA |
| | DSVKGRFTISRDNSKNTLYLEMSSLSDEDTAIYYCVKDVPSMYYIFWSGQGTYWGQGTLVTVSSAST |
| HCBG1-5 | TGVHSEVQLVESGGGLVQPGGSLRLSCAASGFTFSSYAMSWVRQAPGKGLEWVSTISNSGGTTNYA |
| | DSVKGRFTISRDNSKNTLFLQMNSLRAEDTAVYYCAKDLPSRYYDFWSGQGNYWGQGTLVTVSSAS |
| | T |
| HCBG1-6 | TGVHSQVTLRESGPALVKPTQTLTLTCTFSGFSLSTSGMCVSWIRQPPGKALEWLARIDWDDDKYYS |
| | TSLRTRLTISKDTSKNQVVLTMTNMDPVDTATYYCAREMATITGLDYWGQGTLVTVSSAST |
| HCBG1-7 | TGVHSQVTLRESGPALVKPTQTLTLTCTFSGFSLSTSGMCVSWIRQPPGKALEWLALIDWTDDKYYS |
| | TSLKTRLTISKDSSKNEVVLTMTNMDPVDTATYYCARVPPTDYHFDSWGQGTLVTVSSAST |
| HCBG1-8 | TGVHSQVQLVQSGAEVKKPGASVKVSCKASGYTFTSYGISWVRQAPGQGLEWMGWISAYNGNTNY |
| | AQKLQGRVTMTTDTSTSTAYMELRSLRSDDTAVYYCARDTSGYRNPGYGMDVWGQGTTVTVSSAS |
| | T |
| HCBG1-9 | TGVHSEVQLVQSGAEVKKPGESLKISCKTSGYSFASYWIGWVRQMPGKGLEWMGIIYPGDSDTIYTP |
| | PFQGQVTISADTSTRTASLEWRSLRASDSAIYYCATGTFLAVPGFDFWGQGTLVSVSSAST |
| HCBG1-10 | TGVHSEVQLVESGGGLAQPGGSLRLSCAASGFTFSTYSMNWVRQAPGKGLEWVSFISRSSNTIYYAD |
| | SVKGRFTISRDNAKNSLYLQMNSLRDEDTAVYYCARDLQRYGSGSSLGPNYFDYWGQGTVVTVSSA |
| | ST |
| HCBG1-11 | TGVHSQLHLQESGPGLVKPSETLSLTCTASGGSISSSSYYWGWIRQPPGKGLEWIGSVYYSGRTYYNP |
| | SLKSRVTISVDTSKNQFSLKLSSVTAADTAVYYCARRAQYYYDSSGYYDAFDIWGQGTTVTVSSAST |
| HCBG1-12 | TGVHSEVQLVESGGGLVQPGGSLRLSCAASGFTFSTYDMHWVRQATGKGLEWVSAIGTAGDTYYP |
| | GSVQGRFTISRENAKNSLYLQMNSLRAGDTAVYYCARDVFRYDSSGRPRYAFDIWGQGTMVTVSSA |
| | ST |
| HCBG1-13 | TGVHSQVQLQESGPGLVKPSETLSLTCTVSGGSMSSDYWSWIRQPPGKGLEWIGYIYYRGSTNLNPS |
| | LQSRLTMSVDTSKNQFSLKLSSVTAADTAVYYCARETRWNWIDSWGQGTPVTVSSAST |
| HCBG1-14 | TGVHSEVQLVESGGGLIQPGGSLRLSCAASGLTVNSNYMTWVRQAPGKGLEWVSVIYSGGSTFYAD |
| | SVKGRFTISRDNSKNTLYLQMNSLRAEDTAVYYCARDLVDYGMDVWGQGTTVTVSSAST |
| HCBG1-15 | TGVHSDVQLVESGGVVVQPGGSLRLACAASGFTFDDYAMHWVRQAPGKGLEWVSLITRDGGSTHY |
| | ADSVKGRFTISRDNSKNSLYLQMNSLRLEDTAFYYCAKDGRRDGYNHALDNWGQGTLVTVSSAST |
| HCBG1-16 | TGVHSQVQLVESGGGVVQPGRSLRLSCAASGFSFSSHVMHWVRQAPGKGLEWVAVISYDGSNKFY |
| | ADSVKGRFTISRDSSKNTVFLQMNSLRDEDTAMYYCAKGRESTSIGGWFDPWGQGTLVTVSSAST |
| HCBG1-17 | TGVHSQVTLRESGPSLVKPTQTLTLTCTFSGFSLSTSGMCVSWIRQPPGKALEWLARIDWDDDKFYST |
| | SLKTRLTISKGTSRNQVVLTMTNMGPVDTATYYCARTIVGGTNWYFDLWGRGTLVTVSSAST |
| HCBG1-18 | TGVHSQLQLQESGPGLVKPSETLSLTCTVSGGSISSSSYYWGWIRQPPGKGLEWIGNFYYIGSTNYNP |
| | SLKSRVTISVDTSRNQFSLKLTSVTAADTAVYYCARRVMGSGWLDSWGQGTLVTVSSAST |
| HCBG1-19 | TGVHSEVQLVESGGGLVQPGGSLRLACAVSGLILDDYALHWVRLAPGKGLEWVSGINWNSGTIHYA |
| | DSVRGRFTTSRDNAKRSLYLQMNSLRPEDTALYYCAKDMGNGFYYYYGLDVWGQGTSVTVSSAST |
| HCBG1-20 | TGVHSEVLLLESGGGLVQPGGSLRLSCAASGFPFGSYAMSWVRQAPGKGLEWVSVISGSADTTYYA |
| | DSVKGRFTISRDNSKNTLYLQMNSLGAEDTAVYYCAKDAPDVVLVPVPYYHYGLEVWGQGTTVTV |
| | SSAST |
| HCBG1-21 | TGVHSQVQLVESGGGVVQPGRSLRLSCAASGFTFRTYDMHWVRQAPGKGLEWVAVISYDGSIKYFA |
| | DSVKDRFTISRDNSQSTLYLQMNSLRAEDTAVYYCARERSGSYYARVELGYWGQGTLVTVSSAST |
| HCBG1-22 | TGVHSEVQLVESGGGLVQPGGSLRLSCSVSGFTVSSNYMNWVRQAPGKGLEWVSVIYPGGSTFYPD |
| | SVKGRFTISRDDSKNTLYLQMNSLRPEDTAAYYCASSRPPIGQLVPGLDLDWFDPWGQGTLVIVSSA |
| | ST |
| HCBG1-23 | TGVHSEVQLVESGGGLIQPGGSLRLSCAASGLTVSSNYMSWVRQAPGKGLEWVSVMYPGGSTYYA |
| | DSVKGRFTISRDNSKNTLYLQMNSLRAEDTAVYYCARDSYRGGGIWGQGTMVTVSSAST |
| HCBG1-24 | TGVHSQVQLVQSGAEVKKPGSSVKVSCKASGGTFSSYAISWVRQAPGQGLEWMGRIISMFGIANNA |
| | QKFQGRLTITADTSTSTAYMELSSLRSEDTAVYYCARGPYYYDSGGYYLDYWGQGTLVTVSSAST |
| HCBG1-25 | TGVHSEVQLVESGGGLIQPGGSLRLSCAASGFIVSRNYMSWVRQAPGKGLEWVSVIYSGGSTFYADA |
| | VKGRFTISRDNSKNTLYLQMNSLRAEDTAVYYCARDMGGDVFDIWGQGTMVTVSSAST |
| HCBG1-26 | TGVHSQLQLQESGPGLVKPSETLSLTCTVSGGSIYSSDYYWGWIRQPPGKGLEWIGSMHYSGSTYHS |
| | PSLNSRVTISVDPSTNQLSLRLSSATAADTAVYYCAAHITVFGIITGNWFDPWGQGTLVTVSSAST |
| HCBG1-27 | TGVHSEVQLVESGGGLIQPGGSLRLSCAASGFNLSGNYMSWVRQAPGQGLEWVSLIYAGGYAYYA |
| | DSVKGRFTISRDNSKNMFYLQMNSLRAEDTAIYYCARESAAAWGWLDPWGQGTLVTVSSAST |
| HCBG1-28 | TGVHSEVQLVESGGGLVQPGGSQRLSCAASGFTFSSYVINWVRQAPGKGLEWVSAISGSGDITYYAD |
| | SVKGRFTMSRDNSKKTLYLQMNSLRAEDTAVYYCAKDLGYSYGSEIFDYWGQGTLVTVSSAST |
| HCBG4-1 | ATGVHSQVRLVESGGGVVQPGRSLRLSCAVSGFTFSSYAMNWVRQAPGKGLEWVTVISYDGSNKY |
| | YADSVKGRFTISRDNSKNTLYLQMNSLRPDDTAVYYCARARSGSYYSAIDYWGQGTLVTVSSAST |
| HCBG4-2 | ATGVHSEVQLLESGGSLVQPGGSLRLSCAASGFTFSSYAMSWVRQAPGKGLEWVSAISGSGGSTYY |
| | ADSVKGRFTISRDNSKNTLYLQMNSLRAEDTAVYYCAKEAYFTAWLHFPFDPWGQGTLVTVSSAST |
| HCBG4-3 | ATGVHSHVQLVESGGGVVQPGRSLRISCAASGFTFSSYTMHWVRQAPGKGLEWVAVISKEADISYD |
| | GNNKYYADSVKGRFTISRDNSKNTLYLQLNSLRTEDTAVYYCARDRDDYGDPYGMDVWGQGTTV |
| | TVSSAST |
| HCBG4-4 | ATGVHSEVQLVESGGGLVKPGGSLRLSCAASEFTFSNVWMSWVRQAPGKGLEWVGRIKSKTDGGT |
| | TDYAAPVKGRFTISRDDSKNTLYLQMNSLKTEDTGVYYCTTGPTVRGVSVYWGQGTLVTVSSAST |
| HCBG4-5 | ATGVHSQVQLMQSGAEVKKPGASVKVSCKASGDSFTDYYVHWVRQAPGQGLEWMGWISPYSGGT |
| | NYAPKFRGRVTMTRDTSITTAHMELGRLRSDDTAVYFCARSVGYDAFDVWGPGTMVTVSSAST |
| HCBG4-6 | ATGVHSEVQLVESGGGLVKPGGSLRLSCAASGFTFSSYTMNWVRQAPGKGLEWVSSISGAPNYIYY |
| | ADSVKGRFTISRDNAKNSLSLQMNSLRAEDTAVYYCARDLMDYDILTGYYPAGYWGQGTLVTVSS |
| | AST |
| HCBG4-7 | ATGVHSQVQLVQSGAEVKKPGASVKVSCKASGYTFTGYYMHWVRQAPGQGLEWMGWINLNSGGT |
| | NYAQKFQGRVTMTRDTSISTAYMELSRLRSDDTAVYYCARGPPPPAITMIVVTTVWTSWGQGTTVT |
| | VSSAST |
| HCBG4-8 | ATGVHSEVQLVQSGAEVKKPGESLRISCKGSGYSFTSYWISWVRQMPGKGLEWMGRIDPSDSYTNY |
| | SPSFQGHVTISADKSISTAYLQWSSLKASDTAMYYCARLETTVTTDWFDPWGQGTLVTVSSAST |
| HCBG4-9 | ATGVHSEVQLVESGGGLAQPGRSLRLSCRCSGFRFGDYAVNWVRQFPGKGPEWIGFIRTKTFGAATQ |
| | YAASVKGRFTISRDDSQSVAYLQMNRLITDDTAVYYCTIGNWHGSGNNIMHDYWGQGTLVSVSSAS |
| | T |
| HCBG4-10 | ATGVHSQVQLVQSGAEVKKPGASVKVSCKASGYIFTGYYIHWVRQAPGQGLEWMGWINPISGGTN |
| | YAQKFQGRVTATRDTSITTAYMELTSLTSDDTAVFYCARDRWFGKLEGSFDVWGQGTMVTVSSAST |
| HCBG4-11 | ATGVHSQVQLQQWGAGLLKPSETLSLTCAVYDESFSDYYWTWIRQPPGKGLEWIGEINHSGSTNYN |
| | PSLKSRVTISVDTSKNQFSLRLSSVTAADTAVYYCARDTVVVVGAHAFDIWGQGTMVTVSSAST |
| HCBG4-12 | ATGVHSQLQLQESGPGLVKPSETLSLTCIVSGGSISSSSYYWGWIRQPPGKGLEWIGTIYNSGSTHYNP |
| | SLRSRVTISVDTSKSHFSLKLSSVTAADTAVYYCARPQVLYYYGSGSYSHWFDPWGQGTLVTVSSAS |
| | T |
| HCBG4-13 | ATGVHSEVQLVESGGGLVQPGGSLRLSCAASGFTFSSYEMNWVRQAPGKGLEWVSYISSSGSTIYYA |
| | DSVKGRFTISRDNAKNSLYLQVNSLRAEDTAVYYCARGSGWYVGFDYWGQGTLVTVSSAST |
| HCBG4-14 | ATGVHSQVQLVQSGAEVKKPGASVKVSCKASGYTFTGYYIHWVRQAPGQGLEWMGWINPNSGGT |
| | NYAQKFQGRVTMTRDTSISTAYMELSRLRSDDTAVYFCARVTTIPQYDYGDYYYYYGMDVWGQGT |
| | TVTVSSAST |
| HCBG4-15 | ATGVHSQVQLQESGPGLVKPSETLSLTCTVSGGSISSYYWSWIRQPPGKGLDWIGYIYYSGSTNYNPS |
| | LKSRVTISVDTSKNQFSLKLSSVTAADTAVYYCARDLGQDWGQGTLVTVSSAST |
| HCBG4-16 | ATGVHSQVQLQESGPGLVKPSQTLSLTCTVSGGSISSGSSYWSWIRQPAGKGLEWIGRIYTSGSTNYN |
| | PSLKSRVTISVDTSKNQFSLKLSSVTAADTAVYYCARDIFGSGSPPSGVAFDYWGQGTLVTVSSAST |
| HCBG4-17 | ATGVHSEVQLVESGGGLIQPGGFLRLSCAASGLTVSSNYMNWVRQAPGKGLEWVSVIYSGGSTYYA |
| | DSVKGRFTISRDNSKNTLYLQMNSLRAEDTAVYYCARESYGMDVWGQGTTVTVSSAST |
| HCBG4-18 | ATGVHSQVQLVQSGAEVKKPGSSVKVSCKASGGTFSNYAVSWVRQAPGQGLEWMGGIISIFATANY |
| | AQKFQGRVTITADESTSTAYMELSSLRSEDTAVYYCARVKEWGSYYSDSGSHGKNYFDYWGQGTL |
| | VTVSSAST |
| HCBG4-19 | ATGVHSQVQLVQSGAEVKKPGASVKVSCKASGYTFTGYSMHWVRQAPGQGLEWMGWINPNSGGT |
| | NYAQKFQGRVTMTRDTSISTTYMELSRLRSDDTAVYYCARDGYSSSFIPGPRNYYGMDVWGQGTTV |
| | TVSSAST |
| HCBG4-20 | ATGVHSQVHLVQSGAEVKKPGASVKVSCKASGYTFTSYGINWVRQAPGQGLEWMGWISAYNGNT |
| | NYAQKLQGRVTMTTDTSTSTAYMELRSLRSDDTAVYYCARPRGRLITFGGLIVTTLDYYYGMGVW |
| | GQGTTVTVSSAST |
| HCBG4-21 | ATGVHSQVQLVQSGPEVKKPGASVRVSCKTSGHTFTDYWIQWVRQAPGQGLEWMGWIELSTGGTN |
| | YAPKFQGRVAMSSDTSIRTAYMDLWSLTSDDTALYYCATDKADTSWWEFDPWGQGTLVTVSSAST |
| HCBG4-22 | ATGVHSEVQLVESGGGLVQPGASLRLSCAASGFTVSNNYMSWVRQAPGKGLEWVSVIYSGGSTYY |
| | ADSVKGRFTISRHNSENTLYLQMNSLRAEDTAVYYCARFSKLYQWSFDIWGQGTMVTVSSAST |
| HCBG4-23 | ATGVHSEVQLVESGGGLVQPGGSLRLSCAASGFTVSSNYMSWVRQAPGKGLEWVSVIYSGGSTYYA |
| | DSVKGRFTISRHNSKNTLYLQMNSLRAEDTAVYYCARGTHYGSGSYYHWGQGTLVTVSSAST |
| HCBG4-24 | ATGVHSEVQLVESGGGLIQPGGSLRLSCAASGLTVSSNYMNWVRQAPGKGLEWVSVIYSGGSTFYA |
| | DSVKGRFTISRDNSKNTLFLQMNSLRAEDTAVYYCVRDLDYYGMDVWGQGTTVTVSSAST |
| HCBG4-25 | ATGVHSEVQLVESGGGLIQPGGSLRLSCAASGITVVRNYMTWVRQAPGKGLEWVSVIYSGGTTYYA |
| | DSVKGRFTISRDNSKNTMYLQMNSLRAEDTAIYYCARDLEVVGAMDVWGQGTTVTVSSAST |
| HCBG4-26 | ATGVHSEVQLVESGGGLIQPGGSLRLSCAASGVIVSSNYMSWVRQAPGKGLEWVSVIYSGGSTYYA |
| | DSVKGRFTISRDKSKNTLYLQMNSLRAEDTAVYYCAREGPHGMDVWGQGTTVTVSSAST |
| HCBG4-27 | ATGVHSEVQLVESGGGLVQPGGSLRLSCAASGFTILSNYMSWVRQAPGKGLEWVSVIYSGGSTYYA |
| | DSVKGRFTISRHNSKNTLFLQMNSLRVEDTAVYYCARGPVRGVITHFDYWGQGTLVTVSSAST |
| HCBG7-1 | ATGVHSQVQLVESGGGVVRPGRSLRLSCAASGFTFSTYGMHWVRQAPGKGLEWVAIISYDGNNKY |
| | YTDSVKGRFTISRDNSKNMVYLQMNSLRAEDTAVYYCVKSSSGWYLRLGYWGRGTLVTVSSAST |
| HCBG7-2 | ATGVHSEVQLVESGGGLIQAGGSLRLSCTASGFSVSGNYMNWVRQAPGQGLEWVSVIYTDDTTYYP |
| | DSVKGRFTISRDNSKNTLYLQMNSLRAEDTAVYYCAREGRRGQWSWFDPWGQGTLVTVSSAST |
| HCBG7-3 | ATGVHSEVQLVESGGGLIQPGGSLRLSCTASTFTVSSNYMSWVRQAPGKGLEWVSVIYTGGSTYYA |
| | DSVKGRFTISRDNSKNTLYLQVNSLRAEDTAVYYCAGSPVRGVITHFDYWGQGTLVTVSSAST |
| HCBG7-4 | ATGVHSQVQLQESGPGLVKPSETLSLTCIVSGGSVSSSSYYWSWIRQPPGKGLEWIGYVDYSGSTNY |
| | NPSLKSRVTMSVDTSKNQFSLRLSSVTAADTAMYYCARVDRRYCIITSCYIFDYWGQGTLVTVSSAS |
| | T |
| HCBG7-5 | ATGVHSQVQLVESGGGVVQPGRSLRLSCAASGFTFSSYAMNWVRQAPGKGLEWVAVISYDGGNKY |
| | YADSVKGRFTISRDNSKNTLYLQMNSLRAEDTAVYYCARGRDGYYYGMDVWGQGTTVTVSSAST |
| HCBG7-6 | ATGVHSQVQLVQSGAEVKKPGASVKVSCKASGYTFTGYSIHWVRQAPGQGLEWMGWINPYTGDTS |
| | YVQKFQGCVTMTRDTSISTAYMELSRLTSDDTAVYYCARAPPFSSSSIGVDYWGQGTLVTVSSAST |
| HCBG7-7 | ATGVHSQVQLVQSGAEVKKPGASVKVSCKASGYSFSDYYLQWVRQAPGQGLEWMGWINPKSGGT |
| | HYAQKFQGRVTVTRDTSISTTYMELSSLRSDDTAVYYCAKDHCATTNCFEYLYFGMGVWGQGTTV |
| | TVSSAST |
| HCBG7-9 | ATGVHSQVQLQESGPKQVKPSETLSLTCTVSGASMSSYYWFWLRQPAGKGLEWIGRIYSGGSTNYN |
| | PSFKSRVTMSVDTSKNQFFLELSSVTAADTAVYYCARDLTYYGSNGYTNWGQGTLVTVSSAST |
| HCBG7-10 | ATGVHSEVQLVESGGGLIHPGGSLRLSCAASGFTVTSNYMSWVRQAPGKGLEWVSVIYSGGSTYYA |
| | DSVKGRFTISRDNSKNTLYLQMNSLRAEDTAVYYCASSPVRGVITQFDYWGQGTLVTVSSAST |
| HCBG7-11 | ATGVHSEVQLVQSGAEVKKPGESLKISCKGSGYTFTNYWIAWVRQMPGKGLEWMGAIYPGDSDTR |
| | YSPSFQGQVTISADKSISTAYLQWSSLKASDTAMYFCARHDGSKVTTNDYWGQGTLVTVSSAST |
| HCBG7-12 | ATGVHSQVQLVESGGGVVQPGRSLRLSCAASGFTFSIYAMHWVRQAPGKGLEWVAVISYDGSNKY |
| | YADSVKGRFTISRDNSKNTLYLQMNSLRVEDTAVYYCARDRSGSYYRPGYWGQGTLVTVSSAST |
| HCBG7-13 | ATGVHSQVQLVESGGGVVQPGRSLRLSCAASGFTFSSYGMHWVRQAPGKGLEWVAVISYDGNNKY |
| | YADSVKGRFTISRDNSKNTLYLQMNSLRPEDTAVYYCAQTGGSYFGPFDYWGQGTLVTVSSAST |
| HCBG7-14 | ATGVHSEVQLVESGGGLVQPGRSLRLSCAASGFTFEDYAMHWVRQTPGKGLEWVSGISWNGGIIGY |
| | ADSVKGRFTISRDNAKNSLYLQMIRLRAEDTALYYCAKDILYGDYVGGFAYWGQGTLVTVSSAST |
| HCBG7-15 | ATGVHSQVQLVQSGAEVKKPGASVKVSCKASGYTFTSYAISWVRQAPGQGLEWMGWVSAYNGNT |
| | NYAQKLQGRVTMTTDTSTNTAYMELRSLRSDDTAIYYCAIPYSSVTFDCWGQGTLVTVSSAST |
| HCBG7-16 | ATGVHSEVQLVESGGGLVQPGGSLRLSCVASGFTVTNNFMNWIRQAPGMGLEWVAVVYPGGGSDY |
| | SDSVKGRFIISRDNSKNTVYLQMNSLRSEDTAIYYCARDSQPHYYYGVDVWGQGATVTVSSAST |
| HCBG7-17 | ATGVHSEVQLVQSGAEVKKPGESLKISCKVSGYRFTSYWIGWVRQMPGKGLEWMGMIYLSDSDSR |
| | VGPSFRGQVTISADKSISTAYLQWSSLKTSDSAVYYCAKKKDDRAEAVFDYWGQGTLVTVSSAST |
| HCBG7-18 | ATGVHSEVQLMESGGGLIQPGGSLRLSCAASGFIVSSNYMSWVRQAPGKGLEWVSVIYSGGGTDYA |
| | DSVKGRFTISRDNSKNTLFLQMNSLRAEDTAVYYCARGAAMVKELFDYWGQGTQVTVSSAST |
| HCBG7-19 | ATGVHSEVQLVESGGGLVQPGGGSLRLSCAASGFTFSTYWMSWVRQAPGKGLEWVANIKEDGSEK |
| | YYVDSVKGRVTISRDNAKNSLYLQMNSLRVEDTAVYYCARVRWLQGGIDYWGQGTLVTVSSAST |
| HCBG7-20 | ATGVHSQVQLVQSGAEVKKAGASVKVSCKASGYTFTSYDINWVRQASGQGLEWMGWMNPISGNT |
| | GYAQKFQGRVTMTRNTSITTAYMELSSLRSEDTAVYFCARGGRYCSSTTCYSGVGMDVWGQGTTV |
| | TVPSAST |
| HCBG10-1 | ATGVHSEVQLVESGGGLVQPGRSLRLSCAASGFTFDEYAMHWVRQAPGKGLEWVSGISWSSVSIAY |
| | ADSVKGRFTISRDKAKNSLYLQMNSLRAEDTALYYCAKSNSSGLTPFDHWGQGLLVTVSSAST |
| HCBG10-2 | ATGVHSQLQLQESGPGLVKPSETLSLTCTVSGGSISGSSYQWGWVRQPPGKGLAWIAGIHYSGSTYY |
| | NPSLKSRVTISVETSKNQFSLRLYSVTATDTSLYYCARRDGSYFDYWGQGALVTVSSAST |
| HCBG10-3 | ATGVHSQLQLQESGPGLVKPSETLSLTCTVSGDSMSSRSYYWGWIRQPPGKGLEWIGSIYYSGDTYY |
| | DPSLKSRVTISVDTSKIQFTLNLSSVTAADTAVYYCARHVSDGQSIGWLYYFDCWGQGTLVTVSSAS |
| | T |
| HCBG10-4 | ATGVHSQVQLQESGPGFVKPSQTLSLTCTVSGGSLSSGGYYWTWIRQHPGKGLEWIGYISSSGSTFYN |
| | PSLKSRITISIDTSENQFSLKVSSVTAADTAVYYCARLAPFGERRVDQYYLMDVWGQGTTVTVSSAST |
| HCBG10-5 | ATGVHSQVQLQESGPGLVRPSGTLSLTCGVSGGSISSNNWWSWVRQPPGKGLEWIGEIHYSGGTNY |
| | NPSLKSRVTISLDRSTNQFSLRLTSVTAADTAVYYCARAYYYYYMDVWAEGTTVTVASAST |
| HCBG10-6 | ATGVHSQVQLQESGPGLVKPSETLSLTCTVSGGSISSTSYYWSWVRQPPGKGLEWIGYIYYSGNTNY |
| | NPSLKSRVTMSVDTSKNQFSLKLSSVTAADTAVYYCARADRGYCTITDCFIFNYWGQGTLVTVSSAS |
| | T |
| HCBG10-7 | ATGVHSEVQLVESGGGLVQPGESLRLSCTASAFSVNTYDMYWVRQPTGKGLEWVSTIRVTGDTYYP |
| | DSVKGRFTISRESATNSLYLQMNSLRAGDTAIYYCTRGAHYDFWRGYYTDDHHYMDVWGKGTTVT |
| | VSSAST |
| HCBG10-8 | ATGVHSQMELVESGGGLVKPGGSLRLSCASSGFSLGDYYMTWIRQAPGKGLEWVSYISRSDGKTFY |
| | ADSVKGRFTLSTDDGKKSLYLQMNSLRAEDTAVYYCVRGEGGPHDAFDIWGQGTMVTVSSAST |
| HCBG10-9 | ATGVHSEVQLVESGGGLVQPGGSLRLSCAASGFTFRNYWMSWVRQAPGKGLEWVANIKQDGSEKY |
| | YVDSVKGRFTISRDNAKNSLYLQMNSLRAEDTAVYYCVRCGYDDWYFDVWGRGTLVTVSSAST |
| HCBG10-10 | ATGVHSQVQLVQSGAEVKKPGASVKVSCKASGYIFTNYDINWVRQATGQGLEWMGWMNPNSGNA |
| | GYAQKFQGRVTMTRNTSITTAYMELSSLRSEDTAVYYCGRGPTTAKSGVEWFDPWGQGTLVTVSSA |
| | ST |
| HCBG10-11 | ATGVHSEVQLEESGGGLVQPGRSLRLSCVASGFSFDDYAVHWVRQAPGKGLEWVSGINWNSDSTD |
| | YADSVEGRFTISRDNAKKSVYLQMRSLRAEDSALYYCAKDMGWNVFQGLDVWGQGTTVTVSSAST |
| HCBG10-12 | ATGVHSQVQLVQSGAEVKKPGASVKVSCMASGFSFTSFVISWVRQAPGQGLEWMGWISAYNGNTD |
| | YAQKFQGRVTMTTDTSTSTAYMELRSLRSDDTAIYYCARSWSAYYNWFDPWGQGTLVTVSSAST |
| HCBG10-13 | ATGVHSEVQLVESGGDLVKPGGSLRLSCTVSRFTFKNAWMSWVRQAPGKGLEWVGRIKSKADGGT |
| | TDYAAPVRTRFTISRDDSKNTLYLQMNSLTSEDTAVYYCTTDRHVRGTYWGAYYYYGMDVWGQG |
| | TTVTVSSAST |
| HCBG10-14 | ATGVHSEVQLVESGGGLVQPGGSLRLSCAASGFTFSSYWMHWVRQAPGKGLVWVSRINNDGSSTS |
| | YADSVKGRFTISRDNAKNTLYLQMNSLRAEDTAVYYCARAVRISSGYGLYGDSDYFDYWGQGTLV |
| | TVSSAST |
| HCBG10-15 | ATGVHSEVQLVESGGGSVQPGGSLRLSCAASGFTFSPYSMTWVRQAPGKGLEWVSYISSSSSTIHYA |
| | ESVKGRFTISRDNARDSLYLQMNSLKDEDTAVYYCARARGDAFDIWGQGTMVTVSSAST |
| HCBG10-16 | ATGVHSEVQLVQSGAEVKKPGESLRISCKGFGYYFPSYWIAWVRQMPGKGLEWMGIIYPGDSGTRY |
| | SPSFRGQVTISADRSISTAYLQWSSLKASDTAIYYCARPLFYCSGGSCLTGYWGQGTLVTVSSAST |
| HCBG10-17 | ATGVHSEVQLVESGGGLVQPGGSLRLSCAASGFTFSSYCMHWVRQAPGKGLVWVSRIKSDGSGTTY |
| | ADSVQGRFTISRDNAKNALFLQMNSLRAEDTAVYYCARGRRDGNDLAPDYWGQGTLVTVSSAST |
| HCBG10-18 | ATGVHSQVQLVESGGGVVQPGRSLRLSCAASGFPFIISGMHWVRQAPGKGLEWVAVLSFDGSNEYY |
| | ADSVKGRFTISRDNSKNTLYLQIDSLGPEDTAVYYCVKDLGAYASGRLADWGQGTLVTVSSAST |
| HCBG10-19 | ATGVHSEVQLVQSGAEVKKPGESLKISCKGSGYSFTSYWIGWVRQMPGKGLEWMGVIYPGDSDTRY |
| | SPSFQGQVTISADKSISTAYLQWSSLKASDTAMYYCARTQWGYNYGSHFFYMDVWGKGTTVTVSS |
| | AST |
| KCBG1-1 | TGVHSEIVLTQSPGTLSLSPGEGTTLSCRASQSVSSSYLAWYQQKPGQAPRLLIYGASGRATGIPDRFS |
| | GSGSGTDFTLTISRLEPEDFAVYYCQQSESSPYTFGQGTKLEIKRT |
| KCBG1-4 | TGVHSDIQMTQSPSTMSASVGDRVTITCRASQSISNWLAWYQQKPGKAPNLLIYKASSLESGVPSRFS |
| | GSGSGTEFTLTISSLQPDDFATYYCQQYNTYSPITFGQGTRLEIKRT |
| KCBG1-5 | TGVHSDIQMTQSPSTLSASVGDRVTITCRASQNINSRLAWYQQKSGKAPKPLIYKASSLESGVPSRFS |
| | GGGSGTEFTLTISSLQPDDFATYYCQQYNSYSSITFGQGTRLEIKRT |
| KCBG1-6 | TGVHSDIQMTQSPSSLSASVGDRVTITCRASQNIRRYLNWYQQKPGNAPKLLIYAASSLQSGVPSRFS |
| | GSGSGTDFTLTISSLQPDDFATYSCQQSYSTPRTFGQGTKVEIKRT |
| KCBG1-7 | TGVHSEIVLTQSPGTLSLSPGERAALSCRASLPISARYLAWYQQKPGQTPRLLIYGASSRATGIPDRFS |
| | GSGSGTDFTLTISRLDPEDFAVYYCQHYGSSPPLTFGGGTKVEIKRT |
| KCBG1-8 | TGVHSEIVLTQSPATLSLSPGERATLSCRASQSVSSYLAWYQQKPGQAPRLLIYDASNRATGIPARFSG |
| | SGSGTDFTLTISSLEPEDFAVYYCQQRSNWPPWTFGQGTKVEIKRT |
| KCBG1-10 | TGVHSDIQLTQSPSFLSASVGDRVTITCRASQGISSYLAWYQQKPGKAPKLLIYAASTLQSGVPSRFSG |
| | SGSGTEFTLTISSLQPEDFATYYCQQLDTYPRFGGGTKVDIQRT |
| KCBG1-11 | TGVHSEIVLTQSPGTLSLSPGERATLSCRASQSVNSRFLAWYQQKPGQAPRLLIYAASSRATGIPDRFS |
| | GSGSGTDFTLTISSLQPDDFATYYCQQYNSYSTFGQGTKVEIKRT |
| KCBG1-12 | TGVHGDIVMTQSPLSLPVTPGEPASISCRSSQSLLHSNGYNYLDWYLQKPGQSPQLLIYLGSNRASGV |
| | PDRFSGSGSGTDFTLKISRVEAEDVGVYYCMQALQTPLTFGPGTKVDIKRT |
| KCBG1-13 | TGVHSDIVMTQSPDSLAVSLGERATISCKSSQSVSYTSNNKSYLAWYQQKAGQPPKLLIYWASTRES |
| | GVPDRFSGSGSGTDFSLTISSLQAEDVAVYYCQQYHNTPWTFGQGTKVEIKRT |
| KCBG1-14 | TGVHSDIQLTQSPSFLSASVGDRVTITCRASQGISSYLAWYQQKPGKAPKLLIYAASTLQSGVPSRFSG |
| | SGSGTEFTLTIRSLQPEDFATYYCQQLNSYPPYTFGQGTKLEIKRT |
| KCBG1-15 | TGVHSDIQMTQSPSTLSASVGDRVTITCRASQSISSWLAWYQQKPGKAPKLLINRASSLESGVPSRFSG |
| | SGSGTEFTLTISSLQPDDFATYYCQQYSSYPTFGQGTKVEIKRT |
| KCBG1-16 | TGVHSDIQMTQSPSSLSASVGDRVTITCRASQGIGNYLAWFQQKPGKAPKSLIYVASSLQSGVPSKFS |
| | GSGSGTDFILSISSLQPEDFATYYCQHYNAYPITFGQGTRLEIKRTV |
| KCBG1-19 | TGVHGDVVLTQTPLSLSVTPGQPASISCKSNQSLLHSDGKTYLYWYFQRPGQPPQLLIYEVSNPFSGV |
| | PYRFSGSGSGTDFTLKISRVEAEDVGVYYCMQTIHLPRVTFGQGTRLDIKRT |
| KCBG1-20 | TGVHSEIVLTQSPGTLSLSPGERATLSCRASQSVTGNFLSWYQQKPGQAPRLLIYGASSRATGIPDRFS |
| | GSGSGTDFTLTISRLEPEDFAVYYCQHYGSSRRTFGQGTKVEIKRT |
| KCBG1-23 | TGVHSDIQMTQSPSSLSASVGDRVTITCQASQDISNYLNWYQQKPGKAPKLLIYDASNLQIGVPSRFS |
| | GSGSGTDFTFTISSLQPEDIATYYCQQYDNLPQTFGGGTKVEIKRT |
| KCBG1-24 | TGVHSEIVLTQSPGTLSLSPGERATLSCRASQSVGSGYLAWYQQRPGQAPRLLIYGASSRATGIPDRFS |
| | GSGSGTDFTLTISRLEPEDFAVYYCQQYAGSPRTFGQGTRLEIKRT |
| KCBG1-25 | TGVHSDIQLTQSPSFLSASVGDRVTITCRASQGISSYLAWYQQKPGKAPKLLIYAASTLQSGVPSRFSG |
| | SGSGTEFTLTISSLQPEDFATYYCQQLNSSPPKLTFGGGTKVEIKRT |
| KCBG1-27 | TGVHSDIQMTQSPSSLSASVGDRVTITCRASQSMSTYLNWYQQKAEKAPKLLIYAASSLQSGVPSRFS |
| | GSGSGTDFTLTISSLQPEDFATYYCQQSYSMPYTFGQGTKLEVKRT |
| KCBG1-28 | TGVHSDIQMTQSPSSLSASVGDRVTITCRASQSISGYLNWYQHKAGKAPKLLIYAASSLQSGVPSRFS |
| | GSGSGTDFTLTISSLQPEDFATYYCQQSYSTPVFGQGTKLEIKRT |
| KCBG4-1 | TGVHSDIQMTQSPSSLSASVGDRVTITCQASQDISNYLNWYQQKPGKAPKLLIYDASNLETGVPSRFS |
| | GSGSGTDFTFTISSLQPEDIATYYCQQYDYLPLTFGGGTKVEIKRT |
| KCBG4-2 | TGVHSEIVLTQSPGTLSLSPGERATLSCRASQSVSSSYFAWYQQRPGQAPRLLIYGASSRATGIPDRFS |
| | GSGSGTDFTLTISRLEPEDFAVYYCQQYGSSLTFGGGTKVEIKRT |
| KCBG4-6 | TGVHSDIVMTQSPDSLAVSLGERATINCKSSQSVLYSSNNKNYLAWYQQKPGQPPKLLIYWASTRES |
| | GVPDRFSGSGSGTDFTLTISRLQAEDVAVYYCQQYFSTPPSTFGGGTKVEIKRT |
| KCBG4-7 | TGVHGDIVMTQSPLSLPVTPGEPASISCRSSQSLLHSNGYNYLDWYLQKPGQSPQLLIYLGSNRASGV |
| | PDRFSGSGSGTDFTLKISRVEAEDVGVYYCMQALQTPYTFGQGTKLEIKRT |
| KCBG4-9 | TGVHSDIVLTQSPDSLGVSLGERATINCRATETVLYSPHNRNYLAWYQQKAGQPPKLLFYWATTREN |
| | GVPDRFSATGSATDFTLTVNSLQAEDVAVYYCHQYYLVPFTFGPGTRVDIKRT |
| KCBG4-11 | TGVHSDIVMTQSPDSLAVSLGERATINCKSSQSVLYSSNNKNYLGWYQQKPGQPPKLLIYWASTRES |
| | GVPDRFSGSGSGTDFTLTISSLQAEDVAVYYCQQYYTTPLTFGGGTKVEIKRT |
| KCBG4-15 | TGVHSEIVLTQSPGTLSLSPGERATLSCRASQSVSTIYLAWYQQKPGQAPRLLIYAASSRATGIPDRFS |
| | GSGSGTDFTLTISRLEPEDFAVYYCQQYGSSPWTFGQGTKVEIKRT |
| KCBG4-17 | TGVHSEIVLTQSPGTLSLSPGERATLSCRASQSISSTYLAWYQQKLGQAPRLLMYGASSRATGIPDRFS |
| | GSGSGSDFTLTISRLEPEDFAVYFCQQYGSSPGTFGQGTKLEIKRT |
| KCBG4-19 | TGVHSEIVMTQSPAALSVSPGERATLSCRASQSVTSNLAWYQQKPGQAPRLLIYGASTRATGIPARFS |
| | GSGSGTEFTLTISSLQSEDFAVYYCQQFNNWPRGTFGQGTKVEVKRT |
| KCBG4-20 | TGVHGDIVMTQSPLSLPVTPGEPASISCRSSQSLLHSNGYNYLDWYLQKPGQSPQLLIYLGSDRAFGV |
| | PDRFSGSGSGTDFTLKISRVEAEDVGVYYCMQALQTPVTFGGGTKVEIKRT |
| KCBG4-23 | TGVHSEIVMTQSPATLSVSPGERATLSCRASQSVSSNLAWYQQKPGQAPRLLIYGASTRATGIPARFS |
| | GSGSGTEFTLTISSLQSEDFAVYYCQQYNNWPPGLKITFGQGTRLEIKRT |
| KCBG4-24 | TGVHSDIQLTQSPAFLSASVGDRVTITCRASQGISSYLAWYQQRPGKAPKLLIYAASTLQSGVPSRFSG |
| | SGSGTEFTLTISSLQPEDFATYYCQQLNSSPGSLTFGGGTKVEIKRT |
| KCBG4-25 | TGVHSEIVLTQSPGTLSLSPGERATLSCRASQSVPSSYLAWYQQKPGQAPRLLIYGASSRATGIPDRFS |
| | GSGSGTDFTLTISRLEPEDFAVYYCQQYGSPMYTFGQGTKLEIKRT |
| KCBG4-26 | TGVHSDIQMTQSPSSLSASVGDRVTITCQASQDISNYLNWYQQKPGKAPKLLIFDASNSETGVPSRFS |
| | GSGSGTDFTFTISSLQPEDIATYYCQQYDNLPPTFGQGTKLQIKRT |
| KCBG7-1 | ATGVHGDIVMTQSPLSLPVTPGEPASISCRSSQSLLQSNGYNYLDWYLQKPGQSPQLLIYLGSNRASG |
| | VPDRFSGSGSGTDFTLKISRVEADDVGVYYCMQALQTPTFGGGTKVEIKRT |
| KCBG7-2 | ATGVHSEIVLTQSPGTLSLSPGERATLSCRASQSVSSTYLAWYQQKPGQAPSLLIYGASHRATGIPDRF |
| | SGSGSGTDFTLTISRLEPEDFAVYYCQQFGSSPLYTFGQGTKLEIKRT |
| KCBG7-4 | ATGVHSDIVMTQSPDSLAVSLGERATINCKSSLSVLYSSNNKNYLAWYQQKPGQPPKLLIYWASTRE |
| | SGVPDRFSGSGSGTDFTLTISGLQAEDVAVYYCQQYYSTPITFGQGTRLEIKRT |
| KCBG7-5 | ATGVHSDIQLTQSPSFLSASVGDRVTITCRASQGINSYLAWYQQKPGKAPKLLIYAASTLQSGVPSRFS |
| | GSGSGTEFTLTISSLQPEDFATYYCQQLNSLFTFGPGTKVDIKRT |
| KCBG7-6 | ATGVHSDIQMTQSPSSLSASVGDRVTITCRASQAISNYLAWFQQKPGKAPKSLIYAASSLQSGVPSKF |
| | SGSGSGTDFTLTISSLQPEDFATYYCQQYNSYPLTFGGGTKVEIKRT |
| KCBG7-7 | ATGVHGDIVVTQSPLSLPVAPGEPASISCRSSQSLLHSNGYNYLDWYLQKPGQSPQLLIYLGSNRASG |
| | VPDRFSGSGSGTDFTLKISRVEAEDVGIYYCMQSLQTRTFGQGTKVEIKRT |
| KCBG7-9 | ATGVHSDIQMTQSPSTLSASVGDRVTITCRASQSISRWLAWYQQTPGKAPKLLIYKASSLESGVPSRF |
| | SGSGSGTEFTLTISSLQPDDFATYYCQQYNTYFWTFGQGTKVEVKRT |
| KCBG7-11 | ATGVHSEIVLTQSPATLSLSPGERATLSCRASQSVPTYLAWYQQKPGQAPRLLIYDASNRATGVPARF |
| | SGSGSGTDFTLTISSLEPEDFAVYYCQQRRDWPGTFGQGTKLEIKRT |
| KCBG7-12 | ATGVHSDIQMTQSPSSLSASVGDRVTITCQASQDISNYLNWYQQKPGKAPKLLIYDASNLETGVPSRF |
| | SGSGSGTDFTFTISSLQPEDIATYYCQQYGNLPPLTFGQGTRLEIKRT |
| KCBG7-15 | ATGVHSEIVLTQSPGTLSLSPGERATLSCRASQSVSGTFLAWYQQKPGQAPRLLISGASSRATGIPDRF |
| | SGSGSGTDFTLTISRLEPEDFAVYYCQQYGSSRPTFGQGTKLEIKRT |
| KCBG7-18 | ATGVHSDIVMTQSPDSLAVSLGERATINCKSSQSVLYSSNNKNYLAWYQQKPGQPPKLLIYWASTRE |
| | SGVPDRFSGSGSGTDFTLTITSLQAEDVAVYYCQQYYSTPLTFGPGTKVDIKRT |
| KCBG10-1 | ATGVHSDIQMTQSPSSLSASVGDRVTITCRASHSISDYLNWYQQKPGKAPKLLIYAASSLQSGVPSRF |
| | SGSGSGTDFTLTISSLQPKDFATYYCQQSYSNPFTFGPGTKVDIKRT |
| KCBG10-4 | ATGVHGDIVMTQTPLSLSVTPGQPASFSCRSSESLLYSDGKTYLHWYLRKPGQPPQLLIYEVSKRFSG |
| | VPDRFSGSGSGTDFTLKISRVEAEDVGVYYCMQSTQLPWTFGQGTKVEIQRT |
| KCBG10-6 | ATGVHSDIVMTQSPDSLAVSLGDRATINCKSSQNLLFSSNNKNYLAWYQQKPGQPPKLLIYWASTRE |
| | SGVPDRFSGSGSGTDFTLTITSLQAEDVAVYYCQQYYSTPLTFGGGTKVEIKRT |
| KCBG10-12 | ATGVHSEIVLTQSPGTLSLSPGERATLSCRASQSVSSIYLAWYQQKPGQAPRLLIYGASSRATGIPDRF |
| | SGSGSGTDFTLTISRLEPEDFAVYYCQHYGSSPPVTFGQGTKLEIKRT |
| KCBG10-18 | ATGVHSDIQMTQTPSSLSASVGDRVSITCRASQNIHTFLIWYLQRPGKAPNFLIYAASSLQSGVPSRFS |
| | GSGSETDFTLTISSLQPEDFGTYYCQQSYTTPYTFGQGTKLEIKRT |
| LCBG1-3 | TGSWAQSVLTQPPSASGTPGQRVTISCSGSSSNIGSNYVYWYQQLPGTAPKLLIYRNNQRPSGVPDRF |
| | SGSKSGTSASLAISGLRSEDEADYYCAAWDDSLSGVVFGGGTKLTVLGQPKAAPSVTLFPPS |
| LCBG1-9 | TGSWANFMLTQPHSVSDSPGRTVTISCTRSSGDIASSYVQWYQQRPGSVPTTVIHENSERPSGVPDRF |
| | SGSIDTSSNSASLTISGLQTEDEAVYYCQSYDSGRQGVFGGGTKLTVLGQPKAAPSVTLFPPS |
| LCBG1-17 | TGSWAQSVLTQSPSASGTPGQRVTISCSGSSSNIGGNTVNWYQQLPGTAPKLLIYSNNQRPSGVPDRF |
| | SGSKSGTSASLAISGLQSEDEADYYCASWDDSVNGVFGGGTKLTVLGQPKAAPSVTLFPPS |
| LCBG1-18 | TGSWAQSALTQPASVSGSPGQSITISCTGTSSDVGGYNYVSWYQQHPGKAPKVMIYDVRNRPSGVSN |
| | RFSGSKSGNTASLTISGLQAEDEADYYCSSYTSSSTWVFGGGTKLTVLGQPKAAPSVTLFPPS |
| LCBG1-21 | TGSWAQSALTQPASVSGSPGQSITISCTGTSSDVGGYNYVSWYQQHPGKAPKLMIYEVSNRPSGVSN |
| | RFSGSKSGNTASLTISGLQAEDEADYYCSSYTSSITGVFGGGTKLTVLGQPKAAPSVTLFPPS |
| LCBG1-22 | TGSWAQSVLTQPPSVSGAPGQRVTISCTGSSSNIGAGYYVHWYQQHPGTAPKLLIYGNSNRPSGVPD |
| | RFSGSKSGTSASLAITGLQAEDEADYYCQSYDSRLSGWVFGGGTKLTVLGQPKAAPSVTLFPPS |
| LCBG1-26 | TGSWAQSVLTQPPSVSGAPGQRVTISCTGSSSNIGAGYDVHWYQQLPGTAPKLLIYGNNNRPSGVPD |
| | RFSGSKSGTSASLAITGLQAEDEADYYCQSYASSLSVHVVFGGGTKLTVLGQPKAAPSVTLFPPS |
| LCBG4-3 | TGSVTSFELTQPSSVSVSPGQTVTITCSGDVLEKKYVRWFQQRPGQAPILVIYKDTERPSRIPERFSGSS |
| | SGTTVSLIITGAQVEDEADYYCYSVADNMGVFGGGTKLTVLGQPKAAPSVTLFPPS |
| LCBG4-4 | TGSNSQAVVTQEPSLTVSPGGTVTLTCGSTTGAVTSGHYPYWFQQKPGQAPRTLIYDTNNKHSWTPA |
| | RFSGSLLGGKAALTLSGAQPEDEAEYYCLLSYSGAWVFGGGTKLTVLGQPKAAPSVTLFPPS |
| LCBG4-5 | TGSWAQSVLTQPPSASGTPGQRVTVSCSGSSSNILNNYVYWYQQLPGTAPKLLIYRNNQRPSGVPDR |
| | FSGSKSGTSASLAISGLRSEDEADYYCAAWDDSLFVVFGGGTKLTVLGQPKAAPSVTLFPPS |
| LCBG4-8 | TGSWAQSALTQPASVSGSPGQSITISCTGTSSDVGGYNYVSWYQQHPGKAPKLMIYDVSNRPSGVSN |
| | RFSGSKSGNTASLTISGLQAEDEADYYCSSYTRGNYVFGTGTKVTVLGQPKANPTVTLFPPS |
| LCBG4-10 | TGSWAQSALTQPASVSGSPGQSITISCTGTSDDVGGYNLVSWYQQHPSKAPKLMIYAANKRPSGVSN |
| | RFSGSKSGNTASLTISGLQAEDEAHYYCCSYATGSSVVFGGGTKLTVLGQPKAAPSVTLFPPS |
| LCBG4-12 | TGSWAQSVLTQPPSVSGAPGQRITISCTGSTSNIGAGYDVHWYQQLPGTAPKLLIYANSNRPSGVPDR |
| | FSGSKSGTSASLAITGLQAEDEADYYCQSYDSSLSAVWVFGTGTKVTVLRQPKANPTVTLFPPS |
| LCBG4-13 | TGSWAQSVLTQPPSASGTPGQRVTISCSGSNSNIGSNYVYWYQQLPGTAPKLLIYRNNQRPSGVPDRF |
| | SGSKSGTSASLAISGLRSEDEADYYCAAWDDSLSALVFGGGTKLTVLGQPKAAPSVTLFPPS |
| LCBG4-14 | TGSWAQSALTQPASVSGSPGQSITISCTGTSSDVGSYNLVSWYQHHPGKAPKLMIYEVSKRPSGVSNR |
| | FSGSKSGNTASLTISGLQAEDETDYYCCSYAGSSTLVFGGGTKLTVLGQPKAAPSVTLFPPS |
| LCBG4-16 | TGSWAQSALTQPASVSGSPGQSITISCTGTSSDVGGYNYVSWYQQHPGKAPKIMIYDVSNRPSGVSN |
| | RFSGSKSGNTASLTISGLQAEDEADYYCSSHTSSSSPHVVFGGGTKLTVLGQPKAAPSVTLFPPS |
| LCBG4-18 | TGSWAQSALTQPRSVSGSPGQSVTISCTGTSSDVGGYNYVSWYQQHPGKAPKLMIYDVSQRPSGVP |
| | DRFSGSKSGNTASLTISGLQAEDEADYYCCSYAGTYTVIFGGGTKLTVLGQPKAAPSVTLFPPS |
| LCBG4-21 | TGSNSQAVVTQEPSLTVSPGGTVTLTCGSSTGTVTSYHYAYWFQQKPGQVPRVLIYDTSDKPSWTPA |
| | RFSGFLLGDKAALTLSGAQPEDEADYYCLLSYSGVVVFGGGTKLTVLGQPRSAPSVTLFPPS |
| LCBG4-22 | TGSWAQSALTQPRSVSGSPGQSVTISCTGSSTDVGVYNYVSWYQQHPGNAPKLMIFDVSKRPSGVPD |
| | RFSGSKSGNTASLTISGLQAEDEADYYCCSYAGTYTPVVFGGGTKLTVLGQPKAAPSVTLFPPS |
| LCBG4-27 | TGSWAQSALTQPASVSGSPGQSITISCTGTSSDVGGYDFVSWYQQHPGKAPKLMIYDDINRPSGVSNR |
| | FSGSKSGNTASLTISGLQAEDEADYYCSSYTSSSTSSVFGGGTKLTVLGQPKAAPSVTLFPPS |
| LCBG7-3 | ATGSWAQSALTQPASVSGSPGQSITISCTGTSSDVGGYNYVSWYQQHPGKAPKLMIYDVSNRPSGVS |
| | NRFSGSKSGNTASLTISGLQAEDEADYYCSSYTSSSTQVFGGGTKLTVLGQPKAAPSVTLFPPS |
| LCBG7-10 | ATGSWAQSALTQPASVSGSPGQSITISCTGTSSDVGGYNYVSWYQQHPGKAPKLMIYDVSNRPSGVS |
| | NRFSGSKSGNTASLTISGLQAEDEADYYCSSYTSRSPWVFGGGTKLTVLGQPKAAPSVTLFPPS |
| LCBG7-13 | ATGSWAQSALTQPASVSGSPGQSITISCTGTSSDVGGYNYVSWYQQHPGKAPKLMIYEVSNRPSGVS |
| | SRFSGSKSGNTASLTISGLQAEDEADYYCYSYTTSSTPYVFGTGTKVTVLGQPKANPTVTLFPPS |
| LCBG7-14 | ATGSWAQSALTQPASVSGSPGQSITISCTGTSSDVGGYNYVSWYQQHPGKAPKLMIYDVSNRPSGVS |
| | NRFSGSKSGNTASLTISGLQAEDEADYYCSSYTTSSTLVFGTGTKVTVLGQPKANPTVTLFPPS |
| LCBG7-16 | ATGSWAQSGPTQPASVSGSPGQSITISCTGTSTDVGGYHYVSWHQQHPGEAPKLIIYEVSNRPSGVSH |
| | RFSGSKSGNTASLTISGLQAEDEADYYCSSYTKTRTLYVFGTGTMVTVLGQPKANPTVTLFPPS |
| LCBG7-17 | ATGSWAQSVLTQPPSASGTPGQRVTISCSGSSSNIGGNTVTWYRQLPGTAPKLLISTNNQRPSGVPDR |
| | FSGSKSGTSASLAISGLQSEDEADYYCATWDDSLNTYVFGTGTKVTVLGQPKANPTVTLFPPS |
| LCBG7-19 | ATGSWANFMLTQPRSVSESPGKTVTISCTRTSGSIAINYVQWYQQRPGSSPTTVIYEDSQRPSGVPDRF |
| | SGSIDSSSNSASLTISGLKTEDEADYYCQSYDSGSWVFGGGTKLTVLGQPKAAPSVTLFPPS |
| LCBG7-20 | ATGSWAQSVLTQPPSVSGAPGQRVTISCTGSSSNIGAGYDVHWYHQLPGTAPKFLIYGNSNRPSGVP |
| | DRFSGSKSGTSASLAITRLQAEDEADYYCQSYDSSLSGWVFGGGTKLTVLGQPKAAPSVTLFPPS |
| LCBG10-2 | ATGSVTSYELTQPPSVSVSPGQTATITCSGDKLGDQFVSWYRQRPGQSPVVVIYQDIKRPSGIPERFSG |
| | SNSGNRATLTIRGTQALDEADYYCQAGDSDTVLFGGGTQLTVLGQPKAAPSVTLFPPS |
| LCBG10-3 | ATGSVTSYVLTQPPSVSVAPGQTARISCGGDKIGSKNVHWYQQKPGQAPVLVVYDDSDRPSGIPERF |
| | SGSNSGNTATLTITRVEARDEADYYCQVWDRSSVFGTGTKVSVLGQPKANPTVTLFPPS |
| LCBG10-5 | ATGSWAQSLLTQPPSASGTPGQRVTISCSGGSSNIGSNSVCWYQQLPGTAPKLLIYRSNQRPSGVPDR |
| | FSGSKSGTSASLAISGLWSEDEADYYCSAWDDSLRGPVFGGGTKLTVLGQPKAAPSVTLFPPS |
| LCBG10-7 | ATGSWAQSVLTQPPSVSGAPGQRVTISCTGSTSNIGAGYDVHWYQQLPGTAPKLLIYANTNRPSGVP |
| | DRFSGSKSGTSASLAISGLQAEDEADYFCQSYDSRLSAWVFGGGTKLTVLGQPKAAPSVTLFPPS |
| LCBG10-8 | ATGSVTSYVLTQPPSVSVAPGQTARISCGGNNIESKTVHWYQQKPGQAPILVVYDDNDRPSGIPERFS |
| | GSNSGNTATLSISRAEAGDEADYYCQVWDSSRDLRVFGGGTKLSVLGQPKAAPSVTLFPPS |
| LCBG10-9 | ATGSVTSYELTQPPSVSVSPGQTASITCSVDKLGNKYVCWYQQKPGQSPVLVIYQDSKRPSGIPERFS |
| | GSNSGNTATLTISGTQAMDEADYYCQAWDSSTGVFGGGTKLTVLGQPKAAPSVTLFPPS |
| LCBG10-10 | ATGSWAQSVLTQPPSVSGAPGQRVTISCTGSSSDIGAGYNVHWYQQLPGTAPKLLIYGNNNRPSGVP |
| | DRFSGSKSGASASLAISGLQAEDEADYYCQSFDSSLSGFYVFGTGTKVTVLGQPKANPTVTLFPPS |
| LCBG10-11 | ATGSWAQSALTQPASVSGSPGQSITISCTGTTSDVGGYNYVSWYQQHPGKAPKLMIFEVSNRPSGVS |
| | DRFSGSKSGNTASLTISGLQAEDEADYYCTSYRSSSLQGVFGTGTKVTVLGQPKANPTVTLFPPS |
| LCBG10-13 | ATGSWAQSVLTQPPSASGTPGQRVTISCSGSSSNIGSNTVNWYQQLPGTAPKLLIYTNNQRPSGVPDR |
| | FSGSKSGTSASLAISGLQSEDEADYYCAAWDDSLNGLYVFGTGTKVTVLGQPKANPTVTLFPPS |
| LCBG10-14 | ATGSVTSYVLTQPPSVSVAPGQTARITCGGNNIGSKSVHWYQQKPGQAPVLVVYDDSDRPSGIPERFS |
| | GSNSGNTATLTISRVEAGDEADYYCQVWDSSSDPWVFGGGTKVTVLGQPKAAPSVTLFPPS |
| LCBG10-15 | ATGSWAQSVLTQPPSVSAAPGQKVTISCSGNSSNIGNNYVSWYHQLPGTAPKLLIYDNNKRPSGIPDR |
| | FSGSKSGTSATLGITGLQTGDEADYYCGTWDNSLSVGVFGGGTKLTVLGQPKAAPSVTLFPPS |
| LCBG10-16 | ATGSWAQSALTQPPSASGSPGQSVTISCTGTSSDIGAYNYVSWYQQHPDKAPKLMIYEVNNRPSGVS |
| | DRFSGSKSGNTASLTVSGLQAEDEADYYCSSYAGSDNWVFGGGTKLAVLGQPKAAPSVTLFPPS |
| LCBG10-17 | ATGSVTSYALTQPPSVSVSPGQTARITCSGDALPRRYAYWYQQKSGQAPVLVIYEDTKRPSGIPERFS |
| | GSSSGTMATLTISGAQVEDEADYYCYSTDSSDNHRVFGGGTKLTVLGQPKAAPSVTLFPPS |
| LCBG10-19 | ATGSWAQSVLTQPPSASGTPGQRVTISCSGSSSNIGSDHVYWYQQLPGTAPKLFIYRNNQRPSGVPDR |
| | FSGSKSGTSASLAISGLRSEDEADYYCAAWDASLSGYVFGTGTKVTVLGQPKANPTVTLFPPS |

Antibodies isolated from SARS-COV-2 S- and RBD-binding B cells are typically non-polyreactive. Polyreactivity is a feature of antibodies that is selected against in several checkpoints throughout B cell development (Wardemann et al., 2003), but can be generated during affinity maturation (Tiller et al., 2007). Antibody responses to HIV-1 envelope trimer, for example, display elevated levels of polyreactivity, which was suggested to be a means to increase antibody avidity in the setting of a low density of envelope spikes on the HIV surface (Mouquet et al., 2010) and to allow for better binding to divergent HIV-1 envelope strains (Prigent et al., 2018). At the same time, polyreactivity negatively affects the half-life and therefore clinical utility of monoclonal antibodies when considered for clinical use in humans (Horwitz et al., 2013; Shingai et al., 2014).

To assess if antibodies isolated from SARS-COV-2 S- and RBD-binding B cells have an increased frequency of polyreactivity, Applicants tested all 92 isolated antibodies for binding to dsDNA, insulin, bacterial lipopolysaccharide (LPS) and streptavidin-APC by ELISA (Wardemann et al., 2003). Applicants included streptavidin-APC as an antigen to additionally assess any potential off-target binding of B cells to the reagent used to stain the baits used in cell sorting (Methods). 11 of 92 (12%) of all antibodies and 6 of 56 (11%) intermediate or strong binders to SARS-COV-2 S or RBD showed reactivity against two or more of the four polyyreactivity antigens in ELISA (FIG. 9E and Table 3), significantly less than polyreactivity frequencies detected in healthy memory B cells (22.7%, (Tiller et al., 2007)) or HIV-specific B cells (75% (Mouquet et al., 2010)). In addition, Applicants tested all 92 antibodies in an ELISA assay developed to screen for off-target antibody binding against baculovirus lysate in the presence of bovine serum albumin (BSA) (Hotzel et al., 2012) (Methods). None of the antibodies were found to be reactive against baculovirus lysate in this assay (Table 3). Applicants conclude that most antibodies isolated from SARS-COV-2 S- and RBD-binding B cells are not polyreactive when expressed as IgG1 and that intermediate or strong binding to the SARS-COV-2 surface trimer does not require polyreactivity.

Neutralizing antibodies against SARS-COV-2 mostly arise from IgG+ B cells and target the RBD. Applicants next screened all 92 antibodies for neutralizing activity in the pseudotyped virus neutralization assay used on the serum samples (Robbiani et al., 2020; Schmidt et al., 2020) (above and Methods). 27 of 92 antibodies (29%) showed neutralizing activity when tested up to a concentration of at least 25 μg/ml, and neutralizing antibodies were identified from all 4 selected subjects (FIG. 9F and Table 2). IC50 values ranged from 3 ng/ml to 25 μg/ml with a median of 99 ng/ml (FIG. 9F and Table 2). Neutralizers showed at least intermediate binding in ELISA to both SARS-COV-2 S trimer and RBD, with the exception of one antibody, BG4-14, a weak neutralizer with binding by ELISA only to RBD (Tables 2 and 3).

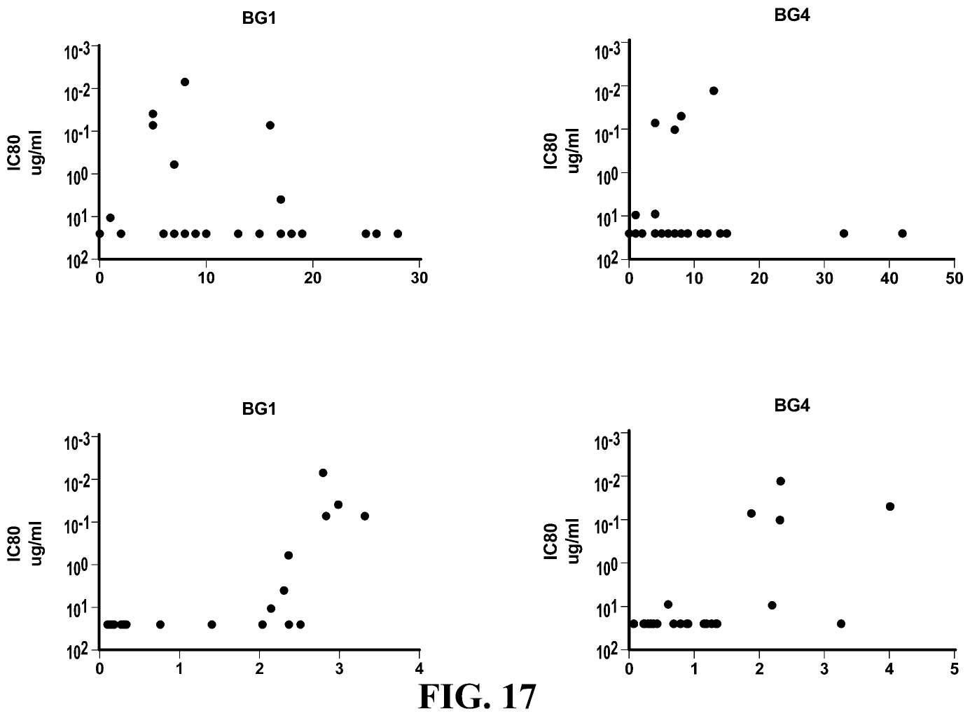

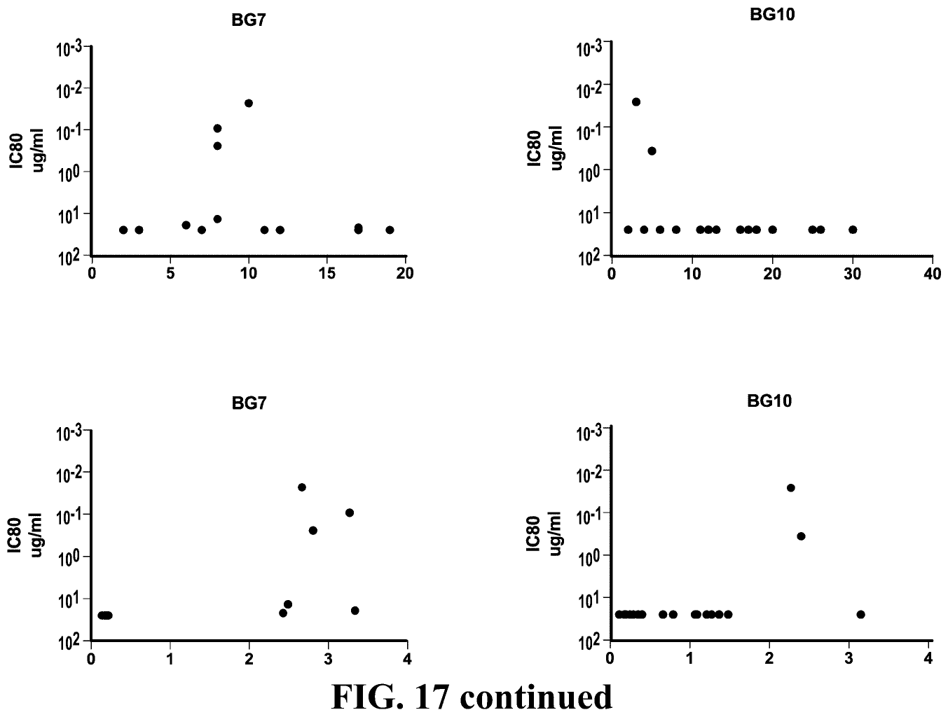

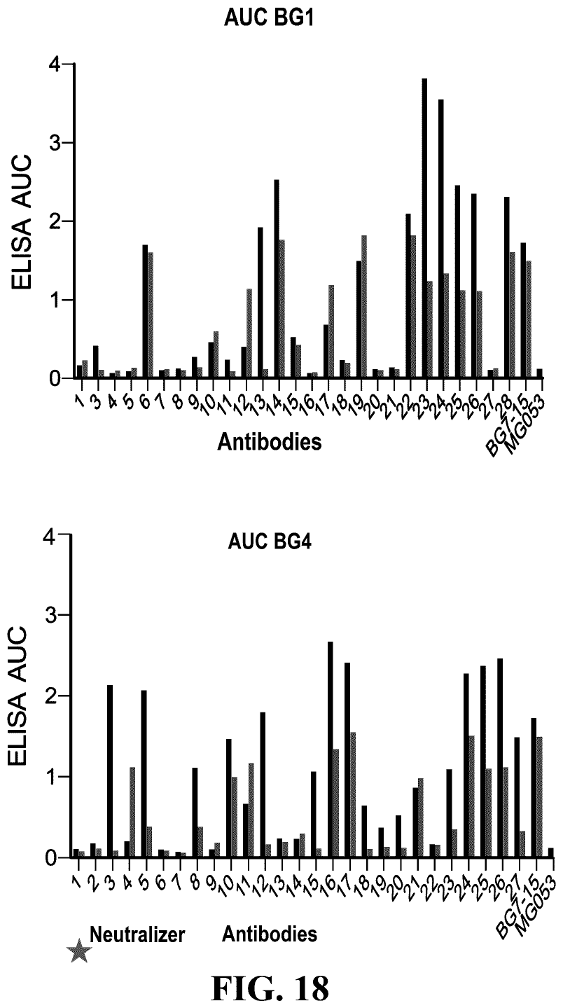

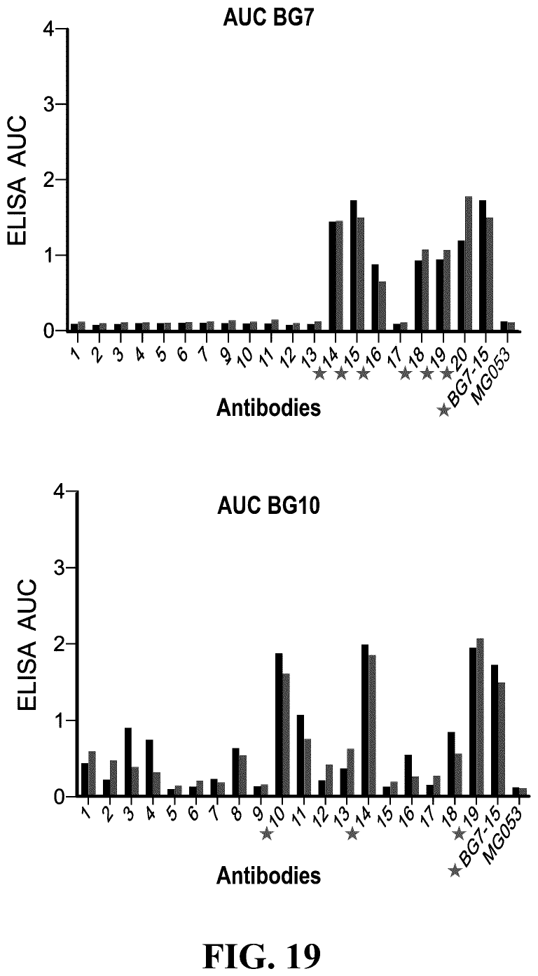

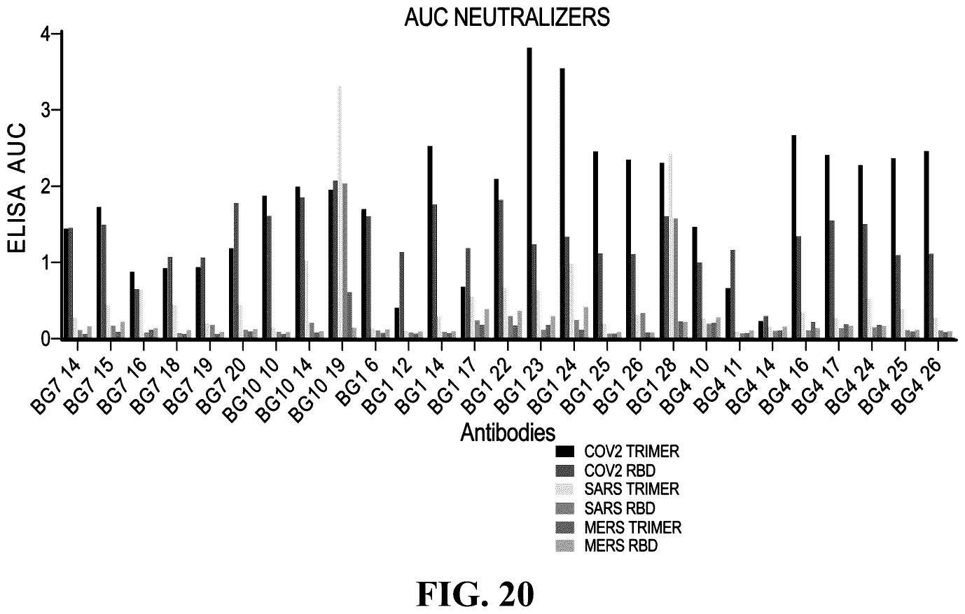

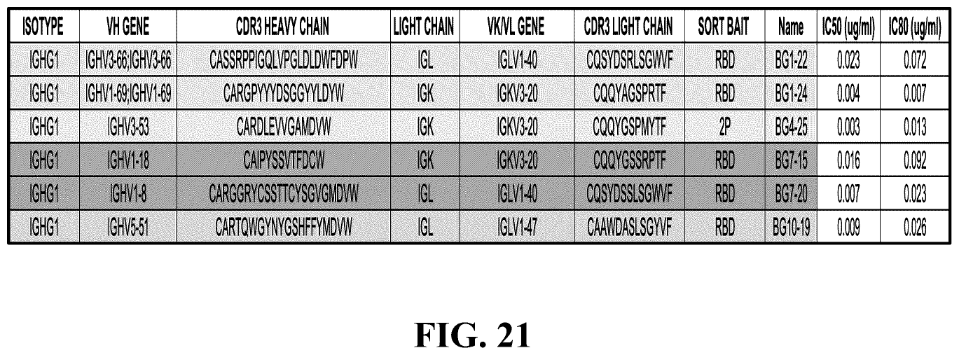

Neutralizing activity was detected for 21 of 42 antibodies (50%) selected from IgG cells compared with 4 of 35 (11%) from IgM/D cells and 2 of 15 (13%) from IgA cells (×2=15.9, P value 0.0004) (FIG. 9G and Table 2). To evaluate if these 27 neutralizers were similarly potent against authentic SARS-COV-2 virus compared with pseudovirus, Applicants tested their activities in a neutralization assay against authentic SARS-COV-2 and found the results between both SARS-COV-2 neutralization assays to be comparable (correlation coefficient (r) for comparison between IC₅₀ values 0.701, P value <0.0001) (FIG. 2B, FIG. 9H and Table 2). Applicants conclude that neutralizing antibodies targeting the RBD of SARS-COV-2 were present in all four selected subjects, mostly arise from IgG⁺ B cells, and are active against both a pseudotyped virus and against authentic SARS-COV-2. See, also, FIGS. 17-20.

Select antibodies retain potent neutralizing activity against circulating variants B.1.1.7 and B.1.351. Several circulating variants of SARS-COV-2, including B.1.1.7 and B.1.351 (Tegally et al., 2020) show decreased sensitivity to some SARS-COV-2 monoclonal antibodies, polyclonal sera from recovered COVID-19 patients (Wibmer et al., 2021) and sera from individuals who have received SARS-COV-2 mRNA vaccines (Wang et al., 2021) (Wu et al., 2021) (Liu et al., 2021). To test if the antibodies described herein retain neutralizing activity against B.1.1.7 and B.1.351 Applicants produced pseudotyped SARS-COV-2 viruses carrying the reported spike mutations in these variants ((Davies et al., 2021) (Tegally et al., 2020) and Methods) and tested antibodies BG10-19, BG1-22, BG4-25 and BG7-15 in a pseudovirus neutralization assay (FIGS. 2E and 9I). All three antibodies neutralized B.1.1.7 with similar potency as WT (D614G) and both BG10-19 and BG7-15 retained neutralization potency against B.1.351 whereas both BG1-22 and BG4-25 showed a 15-fold increase in IC50 values against this variant respectively (FIGS. 2E and 9I). Applicants conclude that BG10-19 and BG7-15 retain potent neutralizing activity against the recently identified circulating variants B.1.1.7 and B.1.351.

Antibody BG10-19 potently neutralizes both SARS-COV-2 and SARS-COV. Antibodies that target conserved epitopes among different betacoronaviruses are the subject of intense investigation given their potential utility in future coronavirus outbreaks. A small set of antibodies that were isolated from SARS-COV-infected individuals have been shown to cross-react with SARS-COV-2 (Pinto et al., 2020a) and vice versa (Robbiani et al., 2020).

To evaluate potential cross-reactivity of the antibodies to other coronaviruses, Applicants tested all 92 neutralizing antibodies for binding to SARS-COV and MERS-COV spike (S) protein and RBD in ELISA (FIG. 2C, 2F and Table 3). Two SARS-COV-2 non-neutralizing antibodies showed strong cross binding to MERS (BG4-23 and BG1-13), one of which (BG4-23) also showed strong cross binding to SARS-COV (Table 3). Three SARS-COV-2 neutralizers (BG1-28, BG10-14, BG10-19) showed strong cross-binding to SARS-COV (Table 3), and BG10-19 also showed weak binding to MERS S (FIG. 2C). Applicants next tested if any of the SARS-COV-2 neutralizers that cross-bind SARS-COV also show neutralizing activity against SARS-COV using a pseudovirus neutralization assay. BG10-19 neutralized SARS-COV potently with an IC50 value of 3 ng/ml compared with 20 ng/ml for antibody S309, a cross neutralizing antibody that was originally isolated from a SARS-COV patient (FIG. 2D, 2G) (Pinto et al., 2020a). BG10-19 also neutralized SARS-COV-2 more potently than S309 with an IC₅₀ of 9 ng/ml compared to 79 ng/ml (Table 2 and (Pinto et al., 2020a)). BG1-28 and BG10-14 did not neutralize SARS-COV and neither did a selection of SARS-COV-2 neutralizers that showed intermediate cross binding to SARS-COV in ELISA (FIG. 2D, 2G and Table 3).

IgA dimerization increases neutralization of SARS-COV-2. Secreted IgA and IgM antibodies can, if expressed with J chain, multimerize and therefore increase overall antibody avidity depending on the density and accessibility of the antigen binding sites (Klein and Bjorkman, 2010). Consistent with this, some IgA dimers of SARS-COV-2 neutralizing antibodies show increased neutralizing potency when compared to their monomeric versions (Wang et al., 2020b). To test the effect of dimerization on neutralizing activity in our antibodies Applicants expressed and purified 13/15 antibodies which were derived from IgA+ B cells (Tables 2 and 3) as IgA monomers and dimers (FIG. 9J, K) and tested them for neutralization of SARS-COV-2 in a pseudotyped neutralization assay. Both antibodies that showed neutralizing activity against SARS-COV-2 when expressed as IgG1 (BG1-23 and BG1-26, Tables 2 and 3) showed similar neutralization potency when expressed as IgA monomers. In dimeric form, their neutralizing activity increased with molar neuralization ratios (MNRs) (Klein and Bjorkman, 2010) of 6 and 8 respectively suggesting an added avidity effect through dimerization (FIGS. 9L, M). 6 of the remaining 11 antibodies that had not shown any neutralizing activity when expressed as IgG1 neutralized SARS-COV-2 either weakly only as dimeric IgA (BG10-5) or both as IgA monomer and dimer with IC50 values ranging from 26 ng/ml (dimeric BG10-8) to 6.5 μg/ml (dimeric BG1-15) (FIGS. 9L, M). MNRs>2 were observed for weakly neutralizing IgA dimer BG1-27 (MNR=3) and potently neutralizing IgA dimer BG10-8 (MNR=10.86) suggesting that in these cases dimerization leads to increased overall avidity of the antibody (Klein and Bjorkman, 2010) (FIGS. 9L, M). Applicants conclude that expression as IgA can increase the neutralizing activity of some SARS-COV-2 antibodies against SARS-COV-2 pseudovirus and that in some cases dimerization can further increase potency by increasing overall antibody avidity.

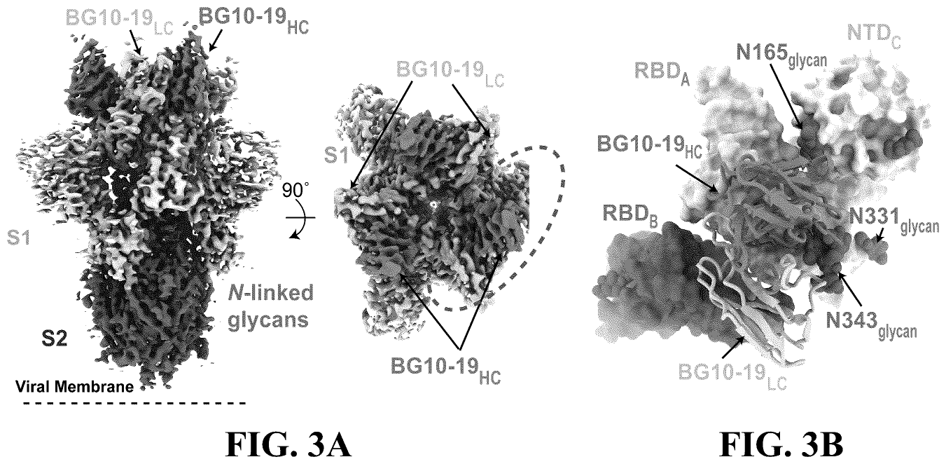

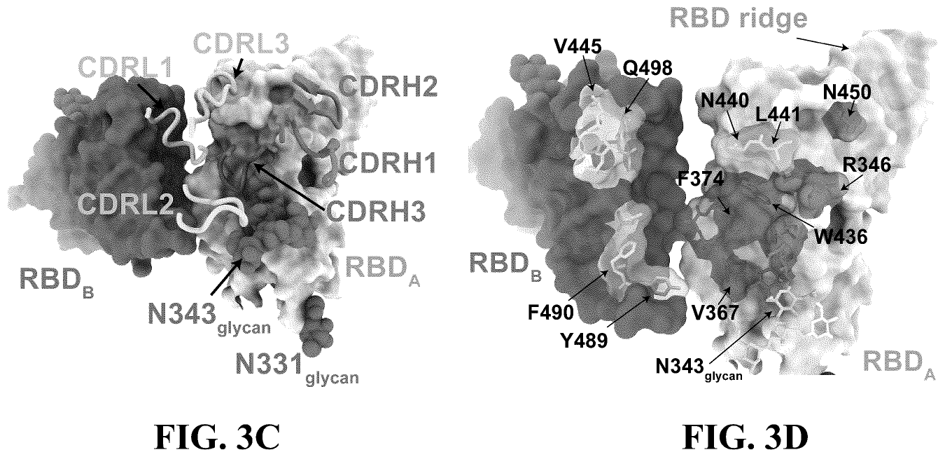

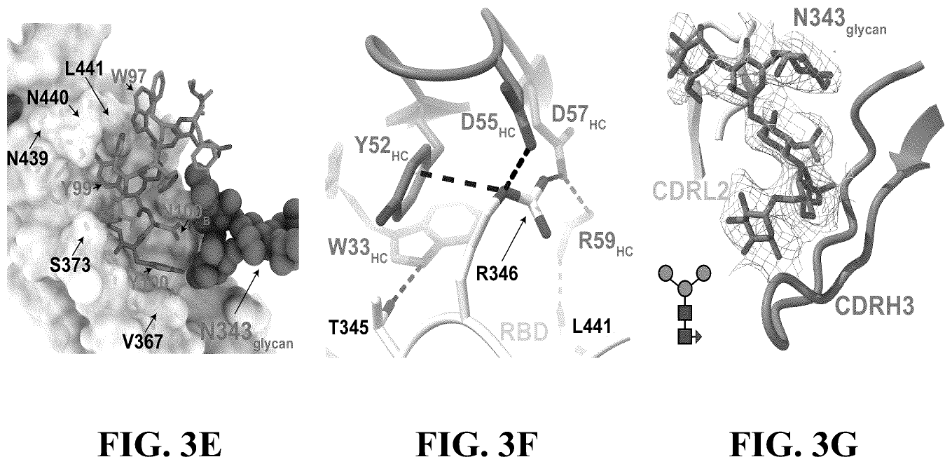

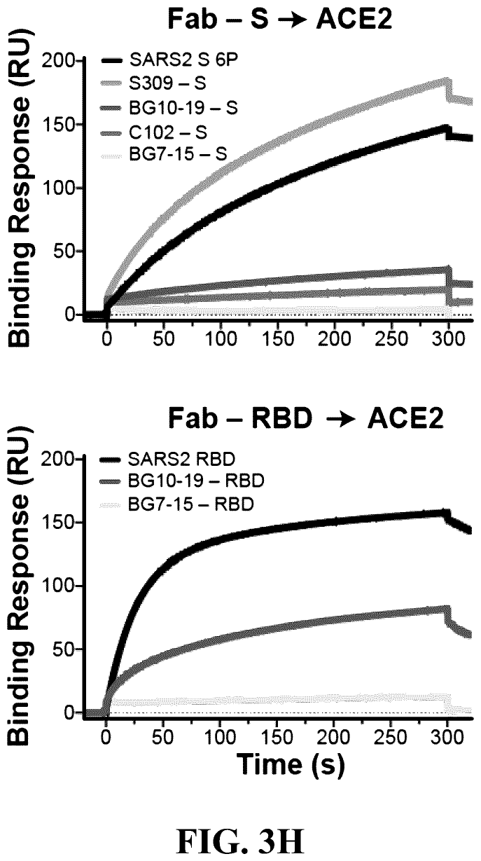

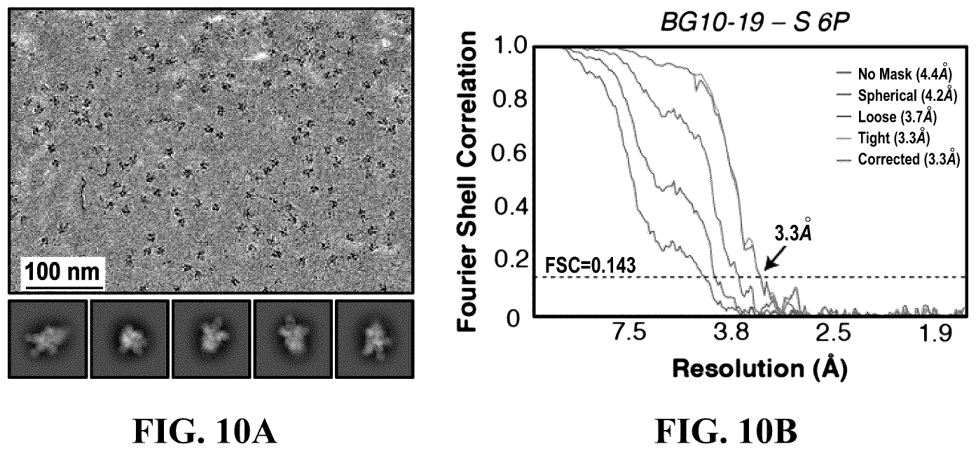

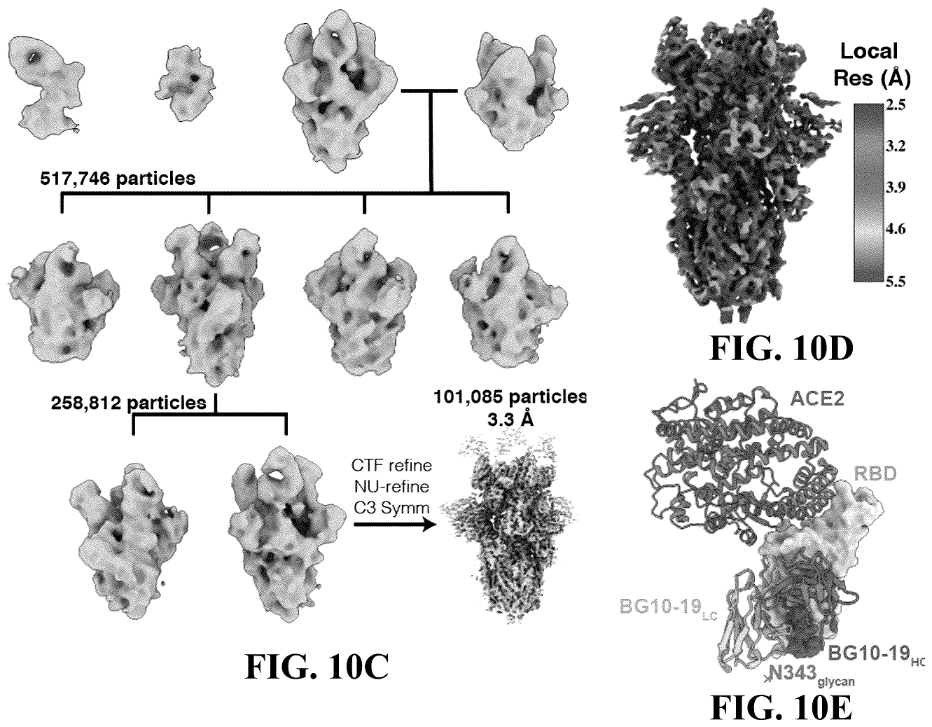

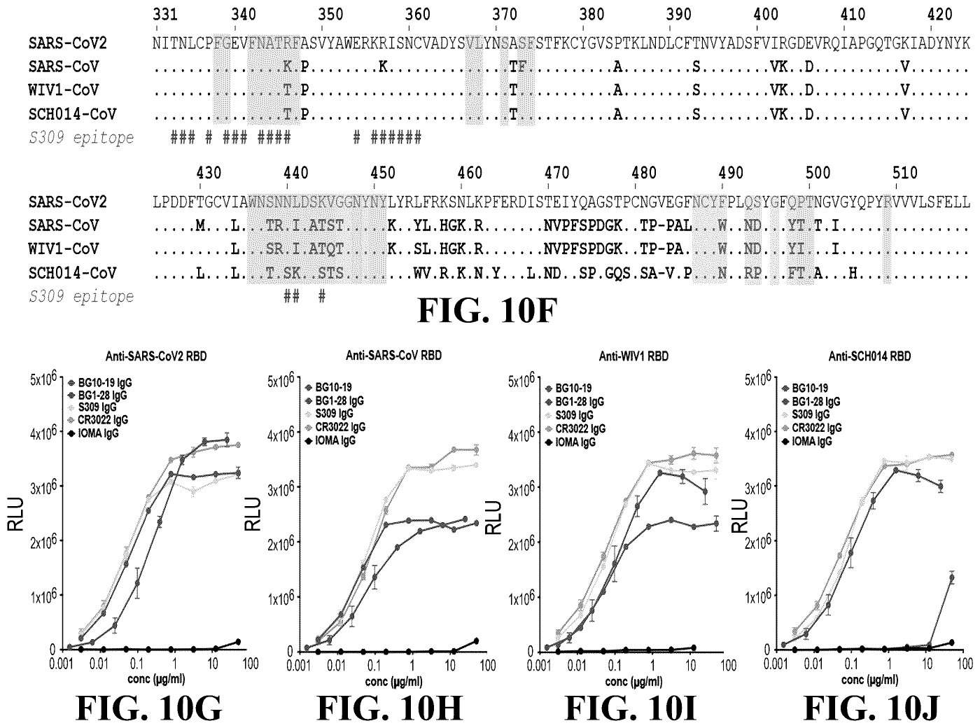

A cryo-electron microscopy structure of a complex between the SARS-COV-2 S trimer and the Fab fragment of cross-neutralizing antibody BG10-19. To understand the mechanism of potent BG10-19-mediated neutralization of both SARS-COV-2 and SARS-COV, Applicants determined a 3.3 Å single-particle cryo-electron microscopy (cryo-EM) structure of a complex between a soluble, stabilized SARS-COV-2 S (Hsieh et al., 2020) and the BG10-19 Fab (FIG. 3, FIG. 10 and Table 5).

The BG10-19-S structure revealed S trimers adopting a closed conformation bound to three BG10-19 Fab fragments (FIGS. 3A and 10A-C). BG10-19 recognizes a quaternary epitope comprising distinct regions from all three protomers, including interactions that bridge two neighboring RBDs and the N165_{NTD}-glycan (FIG. 3B). Specifically, BG10-19 uses five of its six complementarity-determining region (CDR) loops to interact with a proteoglycan epitope focused atop the RBD α1 (residues 338-347) and α2 (residues 364-374) helices, with additional contacts with residues 436-450 (FIG. 3C-G). The CDRH2 and CDRH3 loops mediate the majority of RBD contacts (˜760 Å² of ˜1090 Å² total paratope buried surface area (BSA)), establishing extensive hydrogen bonding and van der Waals interactions with RBD residues (FIG. 3E, G). The N343_{RBD}-glycan (modeled as a complex-type pentasaccharide) at the base of the al helix interfaces with both CDRH3 and CDRL2 loops, including contacts with the core fucose moiety in a manner similar to the cross-reactive SARS-COV monoclonal antibody S309 (FIG. 3F) (Pinto et al., 2020a). The CDRH2 and CDRH3 loops mediate the majority of RBD contacts (˜760 Å² of ˜1090 Å² total paratope buried surface area (BSA)), establishing extensive hydrogen bonding and van der Waals interactions with RBD residues (FIG. 3E,F). Collectively, interactions mediated by CDRH1-3 and CDRL2 loops establish the primary epitope recognized by BG10-19, which does not overlap with the ACE2 receptor-binding motif (RBM) (FIG. 10) and accounts for 87% of BSA on the S trimer (˜925 Å² of peptide BSA and ˜220 Å² glycan BSA).

However, unlike the binding mode of S309, BG10-19 adopts a pose that positions the light chain CDRL1 loop and FWR3 atop the neighboring “down” RBD to lock the S trimer into a closed conformation (FIG. 3A-D and FIG. 10C). This binding orientation is distinct from previously-described neutralizing antibodies that utilize interactions of long CDRH3s to mediate bridging interactions to stabilize the S trimer in a closed, prefusion conformation state (Barnes et al., 2020a; Tortorici, 2020). Additional BG10-19 interactions on the neighboring RBD involve residues 490-498 (FIG. 3D), which overlap with the ACE2 RBM and contribute ˜150 Å² BSA to the total quaternary epitope. Such interactions prevent sampling of the “up” RBD conformation to expose the ACE2 RBM, thus blocking the SARS-COV-2 S 6P trimer binding to immobilized sACE2-CH3 (Tada et al., 2020) in SPR experiments (FIG. 3H). These data suggest that BG10-19 utilizes a neutralization mechanism that inhibits exposure of the ACE2 RBM.

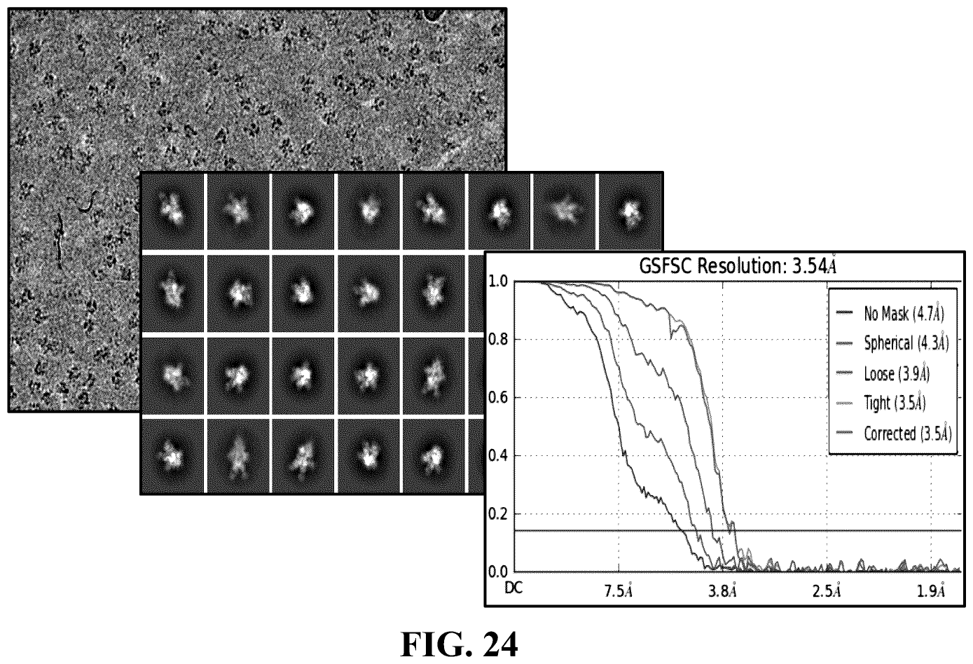

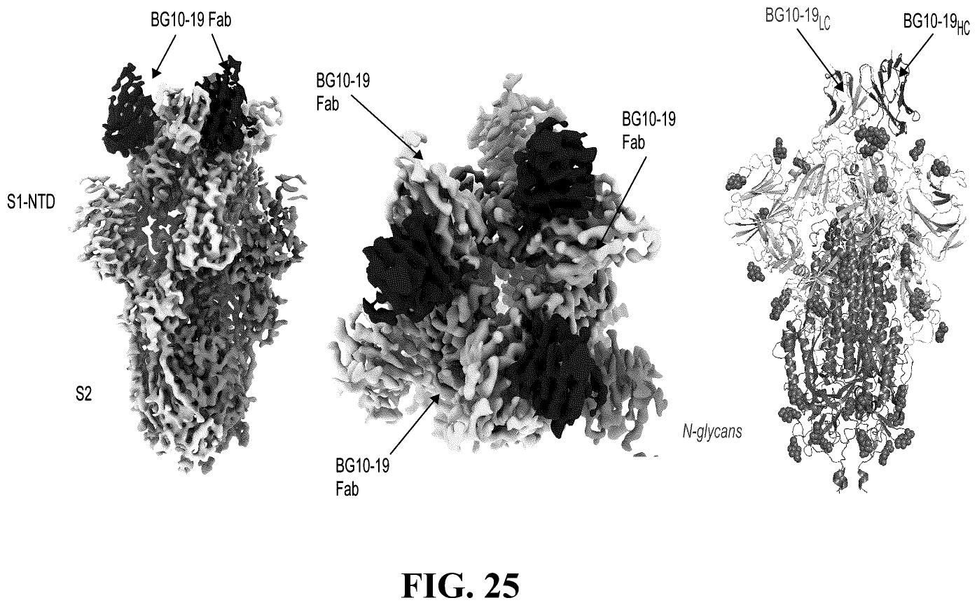

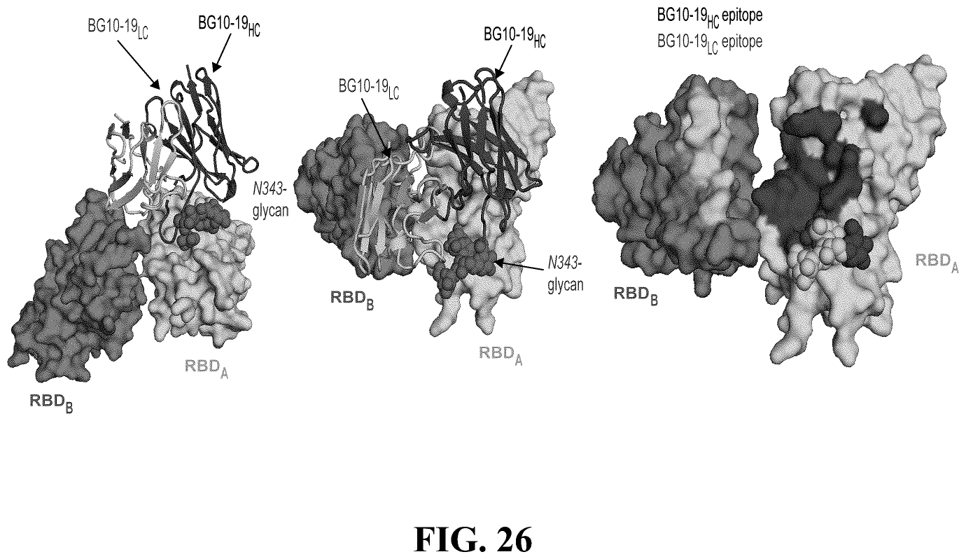

Furthermore, sequence conservation at the BG10-19 epitope explains the potent cross-neutralizing activity against SARS-COV (FIG. 2D, G). The primary binding site for BG10-19 comprises 23 of 29 residues strictly or conservatively substituted between SARS-COV-2 and SARS-COV RBDs (FIG. 10E). Given the highly conserved nature of the BG10-19 epitope, Applicants further characterized the potential of BG10-19 to cross-react with heterologous sarbecoviruses. Bat coronavirus strains WIV1-CoV and SCH014-CoV belong to the clade 1 family of sarbecoviruses and are ACE2-tropic (Li et al., 2003). When Applicants assessed BG10-19 cross-reactivity to these viruses, Applicants observed the binding to WIV1-COV RBD at the same levels as SARS-COV-2 and SARS-COV, but no neutralizing activity (FIG. 10G-J). The lack of neutralizing activity against WIV1-CoV was surprising, given its 95% amino acid sequence identity with SARS-COV (Cohen et al., 2020; Pinto et al., 2020a). Interestingly, key sequence differences occur at RBD positions 346, 439, 440, and 445, and mutations at these positions have been shown to provide immune escape against class 3 anti-SARS-COV-2 neutralizing antibodies and polyclonal sera (Thomson et al., 2020; Weisblum et al., 2020). For example, substitutions at RBD residue R346 have the potential to disrupt cation-pi and hydrogen bond interactions at the antibody-antigen interface (FIGS. 3E, F and 10D). Overall, the findings suggest that the SARS-CoV-2 trimer-specific antibody response includes rare antibodies such as BG10-19 with highly potent cross-neutralizing activity against SARS-COV. See, also, FIGS. 24-26.

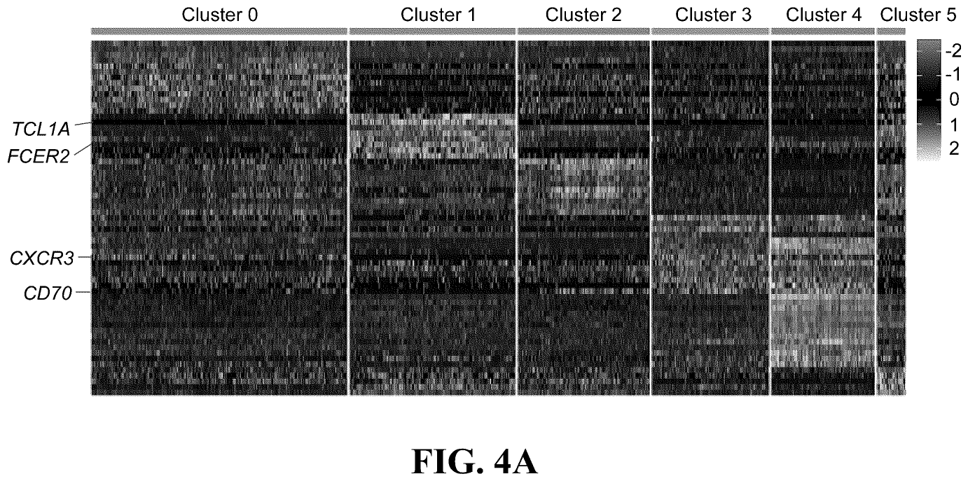

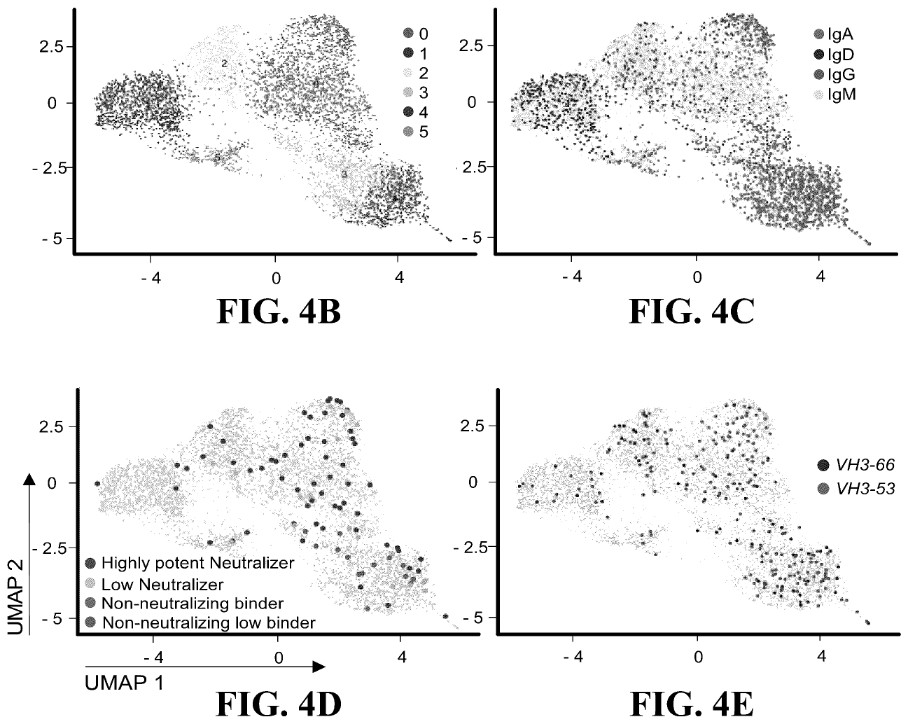

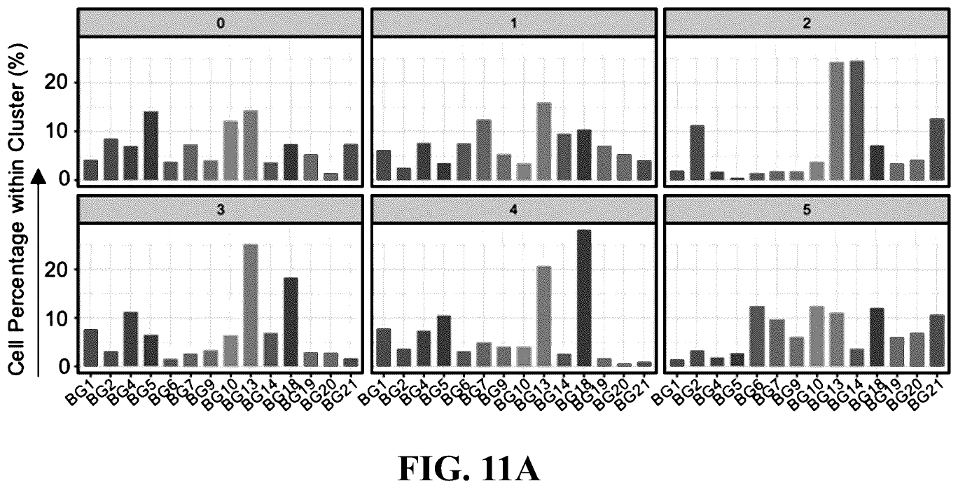

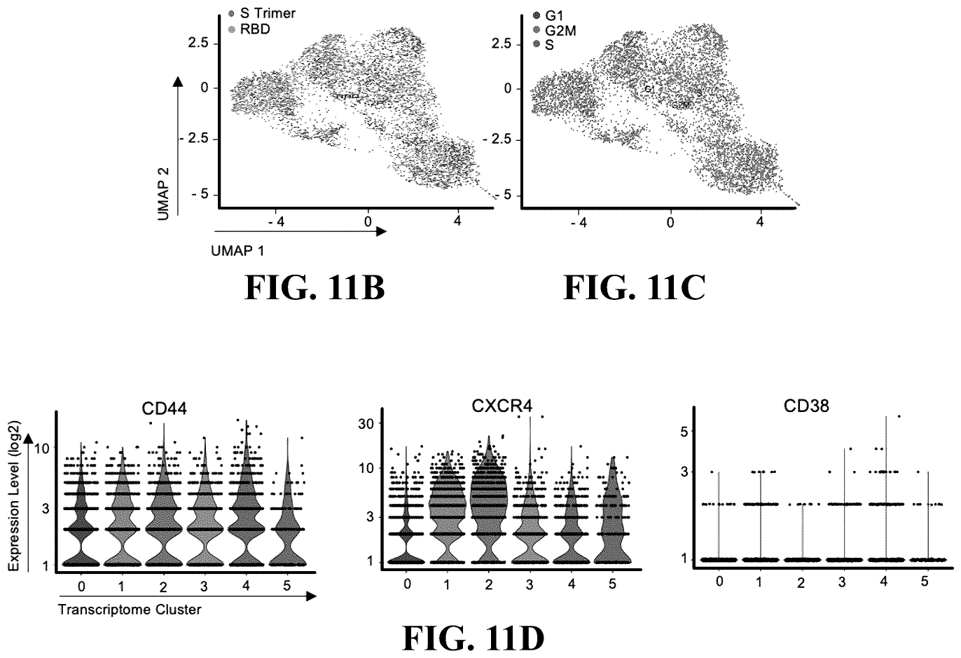

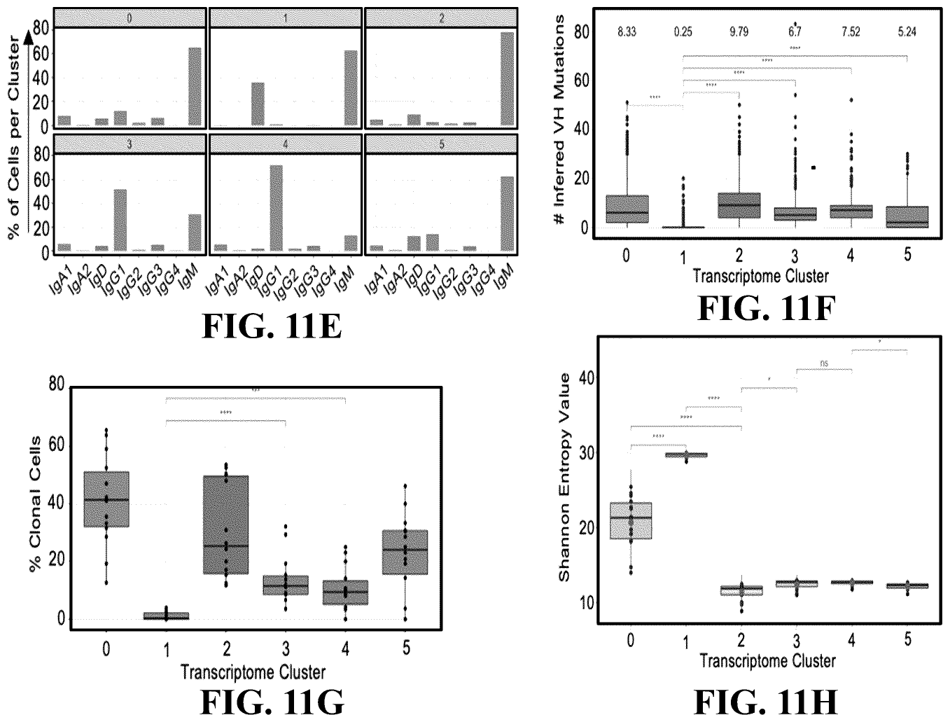

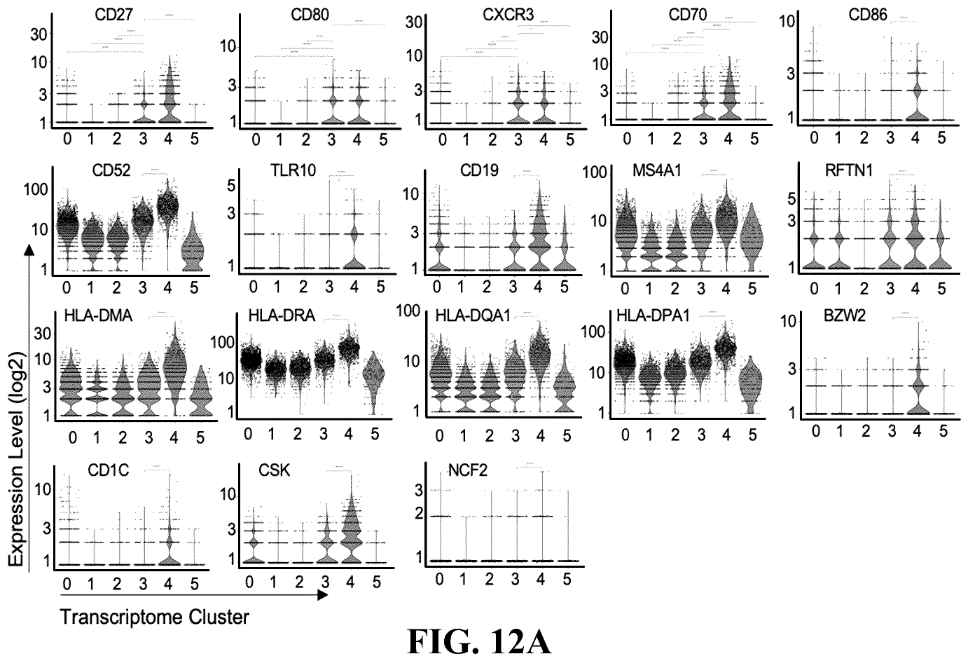

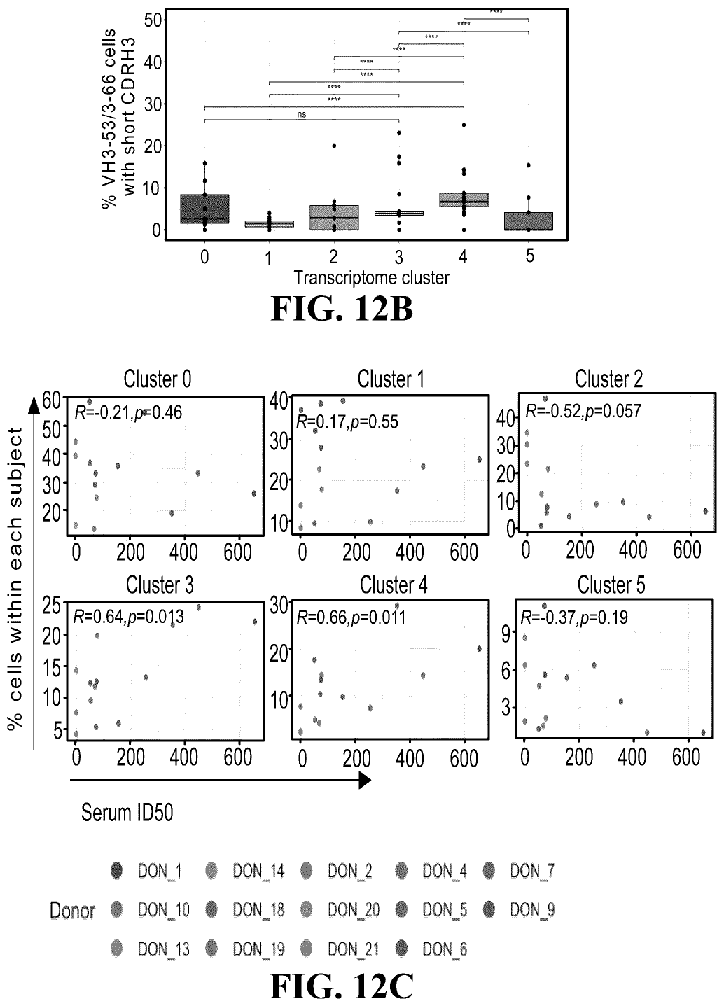

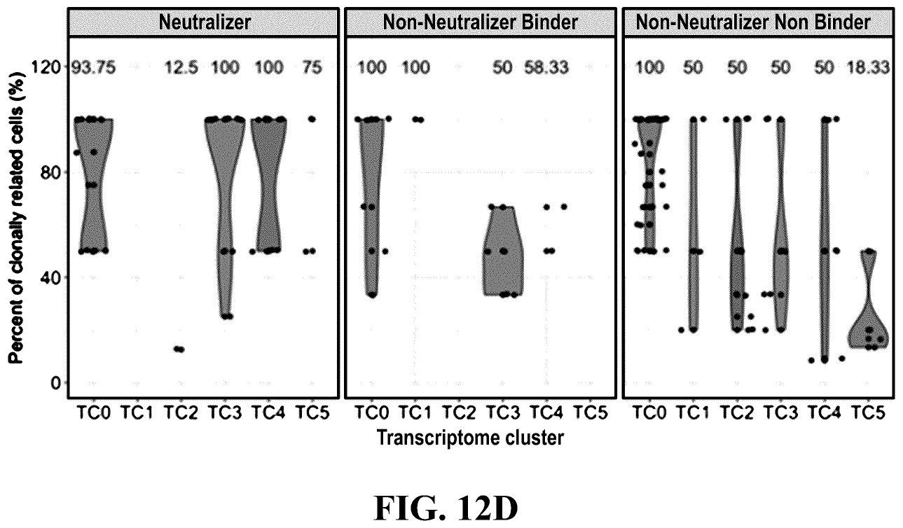

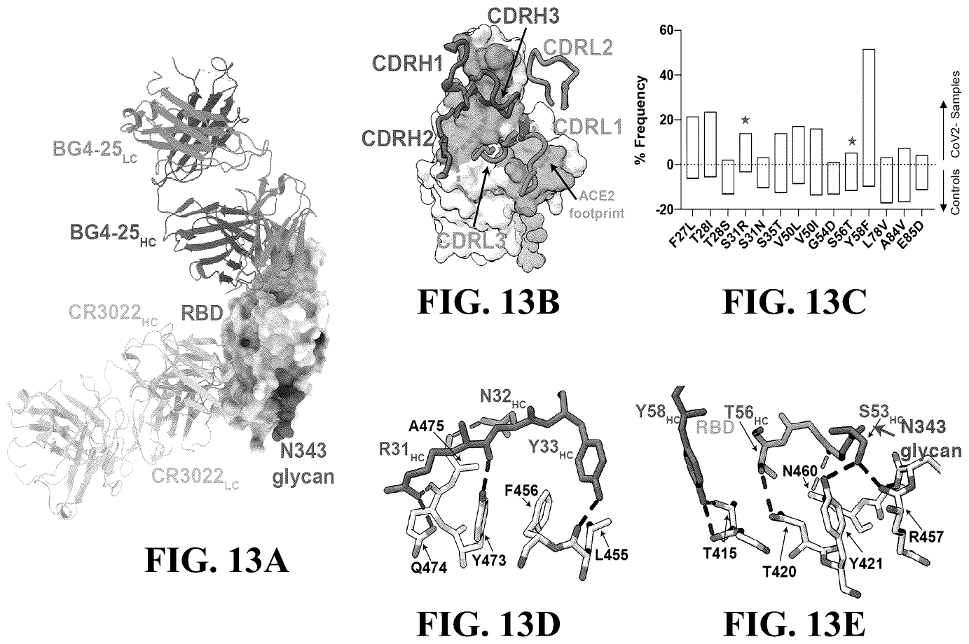

SARS-COV-2 S- and RBD-binding B cell repertoires include different B cell populations. To characterize the transcriptional properties SARS-COV2 and RBD-binding B cells, Applicants performed scRNA-seq paired with VDJ sequencing of B cell populations sorted from the 14 convalescent subjects. These B cells were selected based on expression of CD20, CD19 and SARS-COV-2 S or RBD binding alone without selection for other surface markers or immunoglobulin isotype expression (Methods and above). Applicants profiled 6,113 sorted B cells, revealing 6 distinct transcriptional cell clusters (TCs) (FIGS. 4A, B) that had representation from all donors, though with enrichment of some donors in specific clusters. (FIG. 11A and see below). These clusters were neither dependent on binding to SARS-COV-2 S or RBD (FIG. 11B) nor on cell cycle phase (FIG. 11C). As expected, CD44 and CXCR4 were abundantly expressed throughout all TCs, consistent with their frequent expression among mature B cells (FIG. 11D) (Kremmidiotis and Zola, 1995; Nagasawa et al., 1996; Nie et al., 2004). CD38 expression levels were low throughout all clusters, in keeping with a low frequency of antibody-secreting cells which was expected as B cells were selected based on binding of surface immunoglobulin to SARS-COV-2 S or RBD (Ellebedy et al., 2016) (FIG. 11D). The cell clusters were distinguished by increased expression of specific marker genes (FIG. 4A), such as genes associated with different B cell populations, including naïve and memory B cells, consistent with isotype, mutation status and clonal expansion features of these cells. Specifically, the TC1 marker genes FCER2 (CD23) and TCL1A are known to be highly expressed in mature naïve B cells (FIG. 4A, Table 4) (Horns et al., 2020), consistent with an enrichment in TC1 of IgD and IgM B cells (36% and 63% of cells, respectively, FIG. 11E) with low levels of inferred somatic mutations (FIG. 11F) and minimal clonal expansion (FIG. 11G, H). In TC3 and TC4, CD27 and CD80, both expressed in memory B cells (Moroney et al., 2020; Zuccarino-Catania et al., 2014) were highly expressed (FIG. 12A), and the majority of cells in these clusters were IgG1+(53% and 73% of cells, respectively, FIG. 11E), somatically mutated (FIG. 11F) and more clonally expanded than cells in TC1 (FIG. 11G, H). Cells in TC3 and TC4 are also highly expressed CXCR3, a chemokine receptor found on some class switched B cells that is believed to facilitate migration to sites of inflammation (Moroney et al., 2020; Muehlinghaus et al., 2005) and CD70, which is upregulated on stimulated B cells where its interaction with CD27 on effector T cells plays an important role in antiviral T cell responses (Izawa et al., 2017; van Gisbergen et al., 2011) (FIG. 4A and FIG. 12A).

In the weeks following vaccination or infection with influenza or infection with Ebola, antigen specific B cells can present as antibody secreting cells, memory B cells or “activated B cells” (ABCs), which wane after several weeks and show relatively higher expression levels of CD52, TLR10, CD19 and CD20 (Ellebedy et al., 2016). To test if either TC3 or TC4 include ABCs, Applicants examined the distribution of expression levels in each cell cluster of CD52, TLR10, CD19 and CD20 and 10 other genes that are expressed at higher levels in ABCs compared to memory B cells (FIG. 12A) (Ellebedy et al., 2016). All 14 genes were significantly higher expressed (one-tailed t-test, FDR<0.01) in TC4 than in TC3 (FIG. 12A). Thus, SARS-COV-2 S- and RBD-binding B cell repertoires in recently recovered subjects include ABCs (TC4) and memory B cells (TC3) that are both mostly IgG1⁺, show expansion of B cell clones and somatic hypermutation. Applicants also detected IgD⁺ and IgM⁺ mature naïve B cells (TC1) with low levels of somatic hypermutation, minimal clonal expansion and high expression of CD23 and TCLA1.

TABLE 4
Transcriptome cluster marker genes (related to FIG. 4 and FIG. 11).
For each of the 6 transcriptome clusters the top 30 genes that are
differentially upregulated are displayed. Genes are included only
if their minimum log fold change is greater than 0.25 with a
false discovery rate (FDR) < 0.05. Columns show the gene
name, transcriptome cluster, average log fold change
(avg_logFC) and BH adjusted P values (p_val_adj)
respectively.

| gene | cluster | avg_logFC | p_val_adj |
|---|---|---|---|
| EMP3 | 0 | 0.742674 | 7.4E−231 |
| CIB1 | 0 | 0.704189 | 5.3E−174 |
| LITAF | 0 | 0.696586 | 1.1E−144 |
| LGALS1 | 0 | 0.648787 | 6.46E−87 |
| RHOB | 0 | 0.591643 | 1.06E−85 |
| COTL1 | 0 | 0.586451 | 4.6E−187 |
| SRGN | 0 | 0.570442 | 3.11E−24 |
| PSAP | 0 | 0.528844 | 6.22E−96 |
| YWHAH | 0 | 0.52012 | 1.77E−44 |
| TNFRSF1B | 0 | 0.51046 | 6.22E−57 |
| ITM2C | 0 | 0.509568 | 1.57E−77 |
| IFITM2 | 0 | 0.450464 | 9.91E−88 |
| FCRLA | 0 | 0.444861 | 1.77E−91 |
| SYK | 0 | 0.436202 | 1.16E−85 |
| THEMIS2 | 0 | 0.40288 | 1.43E−83 |
| VOPP1 | 0 | 0.401409 | 6.67E−78 |
| CNFN | 0 | 0.387881 | 2.94E−19 |
| S100A6 | 0 | 0.38559 | 1.87E−38 |
| CBLB | 0 | 0.384918 | 1.32E−81 |
| RGS2 | 0 | 0.381306 | 1.48E−47 |
| GPR183 | 0 | 0.380305 | 2.76E−66 |
| NEAT1 | 0 | 0.379959 | 4.52E−59 |
| KLK1 | 0 | 0.373715 | 5.25E−20 |
| LY6E | 0 | 0.368067 | 7.06E−75 |
| MAP1LC3B | 0 | 0.363525 | 4.22E−38 |
| CLECL1 | 0 | 0.359972 | 4.62E−21 |
| ARL4C | 0 | 0.359298 | 8.06E−19 |
| FCGR2B | 0 | 0.359257 | 1.82E−75 |
| ENC1 | 0 | 0.357722 | 2.84E−59 |
| MYADM | 1 | 1.243403 | 2.3E−225 |
| TCL1A | 1 | 1.225935 | 3.7E−199 |
| IGLL5 | 1 | 1.046009 | 2.42E−69 |
| TUBA1A | 1 | 1.03559 | 2.7E−164 |
| BACH2 | 1 | 0.901778 | 1E−144 |
| CXCR4 | 1 | 0.864938 | 8.9E−189 |
| FCER2 | 1 | 0.827531 | 8.04E−86 |
| CA5B | 1 | 0.783617 | 9.16E−76 |
| FOS | 1 | 0.762213 | 1.7E−99 |
| YBX3 | 1 | 0.73925 | 4.63E−44 |
| TAGAP | 1 | 0.705204 | 4.6E−109 |
| TSC22D3 | 1 | 0.700201 | 2.2E−176 |
| TXNIP | 1 | 0.684033 | 5.7E−154 |
| IQGAP1 | 1 | 0.68283 | 8.19E−82 |
| ITM2B | 1 | 0.682475 | 1.7E−113 |
| IL4R | 1 | 0.673833 | 1.02E−36 |
| CFAP20 | 1 | 0.669236 | 6.18E−48 |
| PIK3IP1 | 1 | 0.666655 | 5.62E−36 |
| APLP2 | 1 | 0.602008 | 3.22E−48 |
| DUSP1 | 1 | 0.601203 | 6.7E−111 |
| ARL4A | 1 | 0.57224 | 7.76E−46 |
| FOXP1 | 1 | 0.559891 | 1.4E−105 |
| SNX9 | 1 | 0.550919 | 2.06E−56 |
| FAM129C | 1 | 0.54711 | 2.8E−55 |
| KLF3 | 1 | 0.541581 | 2.6E−32 |
| SNX29 | 1 | 0.530698 | 5.2E−35 |
| JUN | 1 | 0.514755 | 7.63E−56 |
| TMEM123 | 1 | 0.510121 | 3.92E−29 |
| H1FX | 1 | 0.507778 | 1.98E−11 |
| LINC00926 | 1 | 0.495411 | 2.6E−63 |
| LINC01857 | 2 | 0.89658 | 6.2E−141 |
| LY9 | 2 | 0.805187 | 1.06E−55 |
| ZNF331 | 2 | 0.793354 | 1.3E−112 |
| NR4A2 | 2 | 0.716041 | 8.47E−46 |
| ZFP36 | 2 | 0.683502 | 8.91E−92 |
| JUNB | 2 | 0.621796 | 5.86E−87 |
| CXCR4 | 2 | 0.61256 | 3.1E−119 |
| H3F3B | 2 | 0.5396 | 4.45E−84 |
| RGS2 | 2 | 0.48302 | 2.18E−31 |
| SELENOK | 2 | 0.449154 | 1.37E−49 |
| CD83 | 2 | 0.437254 | 1.85E−45 |
| SAT1 | 2 | 0.429319 | 2.81E−36 |
| BTG1 | 2 | 0.421039 | 6.36E−76 |
| EZR | 2 | 0.415967 | 7.78E−86 |
| IDI1 | 2 | 0.411112 | 1.24E−23 |
| HSPA5 | 2 | 0.401979 | 3.29E−29 |
| FTH1 | 2 | 0.397222 | 1.3E−58 |
| C7orf50 | 2 | 0.388294 | 6.36E−20 |
| ZFP36L2 | 2 | 0.376236 | 1.65E−23 |
| ATP6V0C | 2 | 0.36337 | 3.38E−34 |
| PTP4A1 | 2 | 0.356091 | 4.39E−16 |
| GPR183 | 2 | 0.350688 | 1.52E−24 |
| SELENOM | 2 | 0.347283 | 1.93E−07 |
| RPS3 | 2 | 0.334121 | 3.2E−182 |
| PDCD4 | 2 | 0.333819 | 2.22E−18 |
| PDE4B | 2 | 0.328615 | 8.84E−26 |
| DDIT4 | 2 | 0.328236 | 5.39E−06 |
| RPL3 | 2 | 0.325632 | 8.5E−151 |
| ZFAS1 | 2 | 0.322384 | 3.31E−41 |
| SNX9 | 2 | 0.322189 | 2.42E−32 |
| PDE4D | 3 | 0.617391 | 1.4E−74 |
| GRAMD1C | 3 | 0.5433 | 7.13E−80 |
| TMEM156 | 3 | 0.48219 | 2.27E−75 |
| NFKBIA | 3 | 0.404666 | 4.09E−61 |
| CXCR3 | 3 | 0.390844 | 1.47E−37 |
| CFLAR | 3 | 0.346347 | 7.76E−37 |
| PIM3 | 3 | 0.331141 | 5.81E−32 |
| TKT | 3 | 0.328569 | 3.22E−51 |
| CD70 | 3 | 0.315321 | 1.15E−18 |
| RHOF | 3 | 0.312104 | 2.01E−29 |
| MGAT4A | 3 | 0.304847 | 1.51E−26 |
| HOPX | 3 | 0.298667 | 3.67E−23 |
| CAPG | 3 | 0.296711 | 1.13E−45 |
| MARCKSL1 | 3 | 0.295157 | 1.35E−35 |
| S100A10 | 3 | 0.289821 | 7.32E−59 |
| CD80 | 3 | 0.285129 | 1.57E−19 |
| ZFP36L1 | 3 | 0.28247 | 3.44E−27 |
| COCH | 3 | 0.280678 | 1.25E−25 |
| HMGA1 | 3 | 0.279839 | 9.8E−37 |
| FAS | 3 | 0.27515 | 2.3E−11 |
| BASP1 | 3 | 0.27404 | 6.05E−23 |
| PAPSS1 | 3 | 0.261811 | 1.77E−19 |
| ITGB2-AS1 | 3 | 0.258236 | 4.33E−23 |
| LYPLAL1 | 3 | 0.258037 | 1.67E−18 |
| NME2 | 3 | 0.256528 | 7.04E−27 |
| MIF | 3 | 0.253228 | 2.95E−27 |
| LMNA | 3 | 0.252637 | 6.57E−15 |
| ACTG1 | 4 | 2.187489 | 0 |
| GAPDH | 4 | 1.415694 | 0 |
| S100A10 | 4 | 1.383357 | 7.2E−274 |
| ACTB | 4 | 1.243456 | 0 |
| TKT | 4 | 1.199748 | 2E−301 |
| PFN1 | 4 | 1.193543 | 0 |
| CORO1A | 4 | 1.132533 | 0 |
| HSPA8 | 4 | 1.082111 | 3.2E−263 |
| S100A4 | 4 | 1.056015 | 3.6E−181 |
| HMGA1 | 4 | 1.043888 | 1.8E−253 |
| CNN2 | 4 | 1.042658 | 3.6E−276 |
| SELL | 4 | 1.018083 | 5.4E−218 |
| TMSB10 | 4 | 0.991662 | 0 |
| ARPC1B | 4 | 0.991293 | 4.7E−219 |
| CLIC1 | 4 | 0.952471 | 6.8E−232 |
| RAC2 | 4 | 0.938414 | 1.2E−246 |
| ARHGDIB | 4 | 0.90303 | 1.3E−289 |
| SLC25A5 | 4 | 0.889603 | 6.1E−229 |
| VIM | 4 | 0.859558 | 7.6E−175 |
| PPP1R18 | 4 | 0.84057 | 1.9E−201 |
| PPP1CA | 4 | 0.833401 | 4E−215 |
| CAPZB | 4 | 0.814333 | 3.8E−206 |
| ANXA2 | 4 | 0.803902 | 2.7E−162 |
| CAPG | 4 | 0.793994 | 6.6E−194 |
| CFL1 | 4 | 0.778148 | 1.4E−279 |
| LDHB | 4 | 0.752417 | 7.2E−191 |
| LTB | 4 | 0.736497 | 1.6E−162 |
| TUBB | 4 | 0.725367 | 1E−110 |
| PPIA | 4 | 0.721525 | 1.9E−250 |
| LCP1 | 4 | 0.719491 | 1.6E−135 |
| PTPRC | 5 | 1.302366 | 3.5E−69 |
| MALAT1 | 5 | 1.155559 | 9.5E−118 |
| TMEM123 | 5 | 1.08055 | 0.000249 |
| B4GALT1 | 5 | 1.077235 | 3.42E−24 |
| NFKBID | 5 | 1.057845 | 7.32E−09 |
| CEMIP2 | 5 | 1.021713 | 2.23E−06 |
| LINC00926 | 5 | 1.016311 | 7.84E−33 |
| VPS13C | 5 | 1.015257 | 0.003396 |
| MTRNR2L12 | 5 | 1.012071 | 7.3E−48 |
| SMCHD1 | 5 | 1.00159 | 2.43E−26 |
| NSF | 5 | 0.997369 | 1.08E−09 |
| BACH1 | 5 | 0.983251 | 1.45E−12 |
| PARP14 | 5 | 0.975469 | 3.07E−05 |
| EML4 | 5 | 0.971648 | 1.41E−31 |
| CD69 | 5 | 0.967358 | 0.000444 |
| CASP8 | 5 | 0.954006 | 1.86E−11 |
| BTG2 | 5 | 0.953929 | 3.32E−09 |
| NCOA3 | 5 | 0.949408 | 7.56E−17 |
| SF1 | 5 | 0.949164 | 4.3E−55 |
| REL | 5 | 0.941262 | 1.26E−27 |
| MS4A1 | 5 | 0.940841 | 1.15E−49 |
| AKAP13 | 5 | 0.936896 | 3.65E−06 |
| TMEM131L | 5 | 0.931662 | 0.000402 |
| PRDM2 | 5 | 0.921625 | 7.83E−18 |
| PNN | 5 | 0.918618 | 6.3E−23 |
| STK4 | 5 | 0.914263 | 1.76E−21 |
| PELI1 | 5 | 0.90293 | 0.000183 |
| SRSF4 | 5 | 0.899508 | 2.23E−07 |

TABLE 5
Cryo-EM data collection and refinement statistics (related to FIGS. 3, 5, 6 and. FIGS. 10, 13, and 14).

| | BG10-19 Fab | BG1-22 Fab | BG7-15 Fab | BG7-20 Fab | BG1-24 Fab |
|---|---|---|---|---|---|
| | SARS-COV-2 S 6P | SARS-COV-2 S 6P | SARS-COV-2 S 6P | SARS-COV-2 S 6P | SARS-COV-2 S 6P |
| PDB | XXXX | XXXX | XXXX | XXXX | XXXX |
| EMD | xxxx | xxxx | xxxx | xxxx | xxxx |

Data collection conditions

| | | | | | |
|---|---|---|---|---|---|
| Microscope | Talos Arctica | Talos Artica | Talos Arctica | Talos Artica | Talos Artica |
| Camera | Gatan K3 Summit | Gatan K3 Summit | Gatan K3 Summit | Gatan K3 Summit | Gatan K3 Summit |
| Magnification | 45,000x | 45,000x | 45,000x | 45,000x | 45,000x |
| Voltage (kV) | 200 | 200 | 200 | 200 | 200 |
| Recording mode | counting | counting | counting | counting | counting |
| Dose rate (e⁻/pixel/s) | 13.5 | 13.3 | 13.8 | 13.8 | 13.3 |
| Electron dose (e⁻/Å²) | 60 | 60 | 60 | 60 | 60 |
| Defocus range (μm) | 0.7-2.0 | 0.7-2.0 | 0.7-2.0 | 0.7-2.0 | 0.7-2.0 |
| Pixel size (Å) | 0.8689 | 0.8689 | 0.8689 | 0.8689 | 0.8689 |
| Micrographs collected | 3,807 | 3,386 | 3,065 | 3,717 | 2,609 |
| Micrographs used | 2,860 | 2,720 | 2,464 | 2,830 | 2,060 |
| Total extracted particles | 856,326 | 756,649 | 791,968 | 809,834 | 556,346 |
| Refined particles | 162,522 | 188,436 | 209,924 | 230,863 | 105,245 |
| Particles in final refinement | 101,085 | 71,104 | 130,297 | 135,666 | 84,059 |
| Symmetry imposed | C3 | C3 | C1 | C1 | C1 |
| Nominal Resolution (Å) | | | | | |
| FSC 0.5 (unmasked/masked) | 7.5/3.7 | 7.6/4.1 | 7.8/4.0 | 8.0/4.3 | 8.3/4.5 |
| FSC 0.143 (unmasked/masked) | 4.4/3.3 | 5.9/3.7 | 5.2/3.7 | 6.7/3.8 | 6.7/4.0 |

Refinement and Validation

| | | | | | |
|---|---|---|---|---|---|
| Initial model used | 7K43 | 7K4N | 6XKL | 6XKL | 6XKL |
| Number of atoms | | | | | |
| Protein | 28,466 | 27,798 | 25,662 | 25,238 | 28,871 |
| Ligand | 715 | 873 | 837 | 675 | 779 |
| MapCC (global/local) | 0.81/0.79 | 0.80/0.75 | 0.74/0.73 | 0.79/0.76 | 0.86/0.81 |
| Map sharpening B-factor | 55.7 | 75.3 | 80.7 | 92.4 | 79.1 |
| R.m.s. deviations | | | | | |
| Bond lengths (Å) | 0.002 | 0.005 | 0.01 | 0.015 | 0.001 |
| Bond angles (°) | 0.6 | 0.89 | 0.92 | 0.73 | 0.9 |
| MolProbity score | 2.37 | 2.26 | 2.46 | 2.19 | 2.14 |
| Clashscore (all atom) | 19.7 | 14.8 | 18.1 | 19.24 | 15.2 |
| Poor rotamers (%) | 1.3 | 1.1 | 2.4 | 0.32 | 0 |
| Ramachandran plot | | | | | |
| Favored (%) | 94 | 89.9 | 93.7 | 96.63 | 92.9 |
| Allowed (%) | 5.6 | 10.1 | 6.3 | 3.24 | 6.5 |
| Disallowed (%) | 0.4 | 0 | 0 | 0.13 | 0.6 |

Memory B cells and activated B cells are enriched for cells that generate binding and neutralizing antibodies. To test if antibodies with high levels of binding to or neutralization of SARS-COV-2 were preferentially isolated from B cells in certain TCs, Applicants identified the clusters from which the 92 antibodies Applicants produced and tested are derived. Given that antibodies were selected for production and testing only based on representation of expanded clones (72/92) or randomly selected mostly IgG+ or IgA+ singlets (20/92) all transcriptional clusters are not equally represented among the 92 antibodies (Table 3). However, 20 of 33 (60%) neutralizers, 14 of 15 (94%) potent neutralizers (IC50≤0.1 μg/ml) and 5 of 10 (50%) non-neutralizing high binders were derived from cells in TCs 3 and 4 (FIGS. 4D and 9L, Tables 2 and 3), whereas 36 of 49 (73%) of the low binders belonged to TCs 0 and 2 (FIG. 4D and Table 3). The majority of neutralizers from TCs 3 and 4 were derived from singlets or small clones and did not have clonal members in other TCs among the cells Applicants sampled (FIG. 12D, Table 3). Applicants further tested the clusters for enrichment in class 1 neutralizers (VH3-53/VH3-66 antibodies with short CDRH3 regions (Barnes et al., 2020a) and see above). VH3-53/VH3-66 antibodies were overall significantly enriched in TC3 and TC4 compared to TCs 1, 2 and 5 (FIGS. 4E and 12B, two-tailed t-test, P<=0.0001), and all (8/9) VH3-53/VH3-66 neutralizing antibodies with CDRH3 regions shorter than 14 amino acids according to IMGT definition (Lefranc et al., 2015) were derived from B cells that belonged to either TC3 or TC4 (Tables 2 and 3). Note that the difference between TC3 and TC0 did not reach statistical significance. Applicants conclude that both strongly binding and neutralizing antibodies against SARS-COV-2 are frequently isolated from cells with a transcriptional profile consistent with memory B cells and ABCs.

Frequency of SARS-COV-2 S- and RBD-binding ABCs and memory B cells correlates with serum neutralizing activity. While memory B cells and antibody secreting plasma cells are two separate B cell compartments that are regulated differently (Leyendeckers et al., 1999), memory B cells and plasma cells can originate from the same germinal center reaction, memory B cells can become antibody secreting plasma cells upon antigen binding and stimulation (Victora and Nussenzweig, 2012) and antibodies derived from memory B cells against HIV have been detected in serum from matched patients using mass spectrometry (Scheid et al., 2009a). Whether the frequency of memory B cells among SARS-COV-2 S- and RBD-binding B cells correlates with plasma neutralizing activity in recently recovered subjects is not known. To test this, Applicants measured if the frequency of cells from any TC among SARS-COV-2 S- and RBD-binding B cells correlated with serum neutralization in the 14 study subjects and found that the frequency of only TC3 and TC4 cells correlated positively with serum neutralization against SARS-COV-2 in a pseudovirus assay (Pearson's r=0.64 and 0.66, P=0.013 and 0.011, respectively) (FIG. 12C) and this correlation was not significantly affected by correcting for time since COVID-19 diagnosis (Pearson's r=0.69 and 0.76, P=0.017 and 0.007, respectively). Applicants conclude that expansion of both SARS-COV-2 binding memory B cells and ABCs correlate with serum neutralization titers against SARS-COV-2 in convalescent COVID-19 patients.

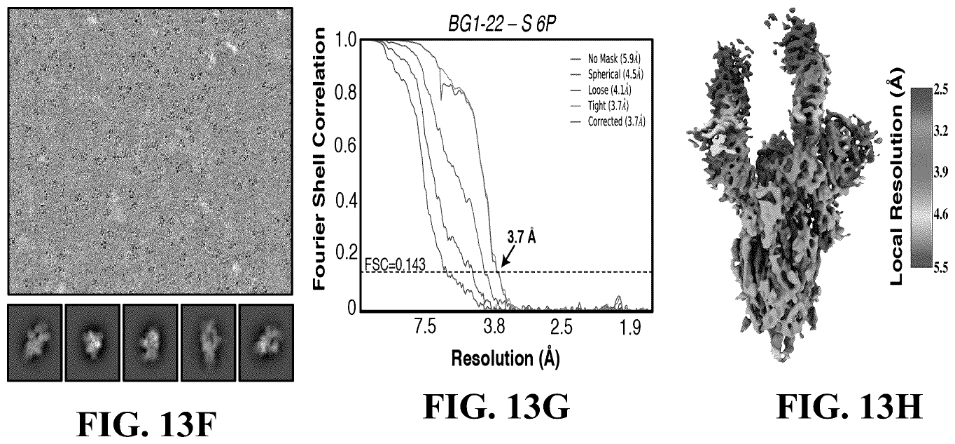

VH3-53/VH3-66 antibodies with long CDRH3s can adapt class 1 antibody structural poses. Given the enrichment of VH3-53/VH3-66-encoded antibodies in TC3 and TC4 that correlated with strong binding and potent neutralization of SARS-COV-2, Applicants selected two antibodies which were coded by cells in these clusters with distinct CDRH3 lengths for structural characterization of their Fab fragments complexed with SARS-COV-2 RBD or stabilized S trimer. Applicants solved a 3.0 Å crystal structure of Fabs from BG4-25 (VH3-53-encoded with 12 aa CDRH3, IC₈₀=13 ng/ml) and the SARS-COV antibody CR3022 (Tian et al., 2020) in complex with SARS-COV-2 RBD (FIG. 13A and Table 6). Consistent with the binding mode of class 1 neutralizing antibodies (Barnes et al., 2020b; Yuan et al., 2020), BG4-25 recognizes a RBD epitope that overlaps with >90% of residues in the ACE2 RBM (FIG. 13B), which is only fully accessible with “up” RBD conformations. Two V-gene encoded regions in class 1 neutralizing antibodies prominently contribute to epitope recognition; the ³¹SNY³³ CDRH1 and ⁵³SGGS⁵⁶ CDRH2 sequence motifs, which take part in extensive hydrogen bond interactions at the RBD interface (Yuan et al., 2020; Tan et al., 2021; Wu et al., 2020b). Analysis of inferred somatic mutations in 93 VH3-53/VH3-66 antibodies coded in the collection of SARS-COV-2 binding B cells with CDRH3 less than 14 amino acids (IMGT definition; (Lefranc et al., 2015) revealed frequent inferred mutations in these motifs, including S31R and S56T in the antibody heavy chain positions 31 and 56, respectively, which are observed in BG4-25 (FIG. 13C-E). VH3-53/VH3-66-encoded antibodies from SARS-COV-2 binding B cells with short CDRH3s were also enriched for inferred mutations Y58F, F27L, and T28I, with the latter two mutations reported to increase neutralization potency in this class of antibodies (Hurlburt et al., 2020). Despite lacking these specific mutations, BG4-25 shows exceptional neutralizing activity, which is potentially explained by the F27I and S31R CDRH1 loop mutations that provide increased van der Waals interactions along the antibody-RBD interface (FIG. 13D, E).

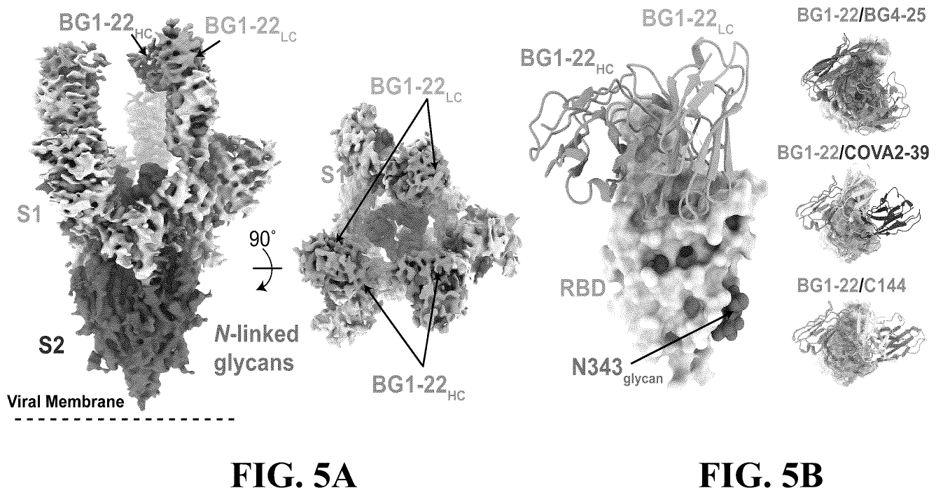

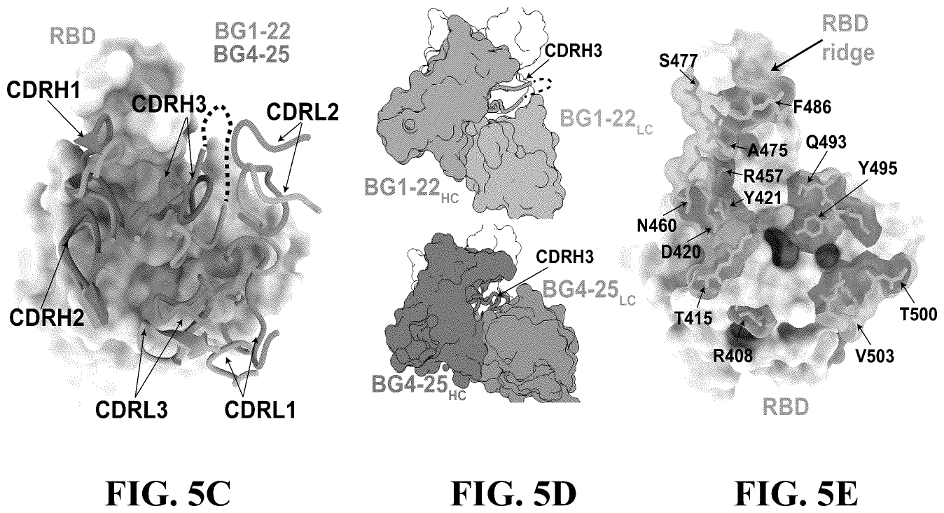

In addition to the recurrent, class 1 neutralizing antibodies defined by VH3-53/VH3-66-encoded gene segments and short CDRH3s (e.g., BG4-25), a subset of VH3-53/VH3-66-encoded neutralizing antibodies have been described with CDRH3 lengths >15 amino acids which would seemingly be incompatible with the binding mode of the recurring class 1 antibodies. A 3.7 Å cryo-EM structure of BG1-22 (VH3-53-encoded with 21 aa CDRH3, IC₈₀=72 ng/ml, Table 2) Fab complexed with stabilized S trimers revealed binding of BG1-22 to “up” RBD conformations (FIGS. 5A and 13F-H). Surprisingly, the binding orientation of BG1-22 was consistent with class 1 antibody binding modes (FIG. 5B), which contrasts with previously-characterized VH3-53/VH3-66-long CDRH3 antibodies (Barnes et al., 2020a) (Wu et al., 2020a). To accommodate this binding mode, the 21-residue long CDRH3 of BG1-22 flips outward towards RBD residue Q493 resulting in a slight rotation of the antibody light chain relative to the canonical class 1 RBD-binding antibodies (FIG. 5C-E). The displaced CDRH3 conformation makes few sidechain interactions with the RBD, such that 6 residues of the CDRH3 loop are disordered (FIG. 5C, D).

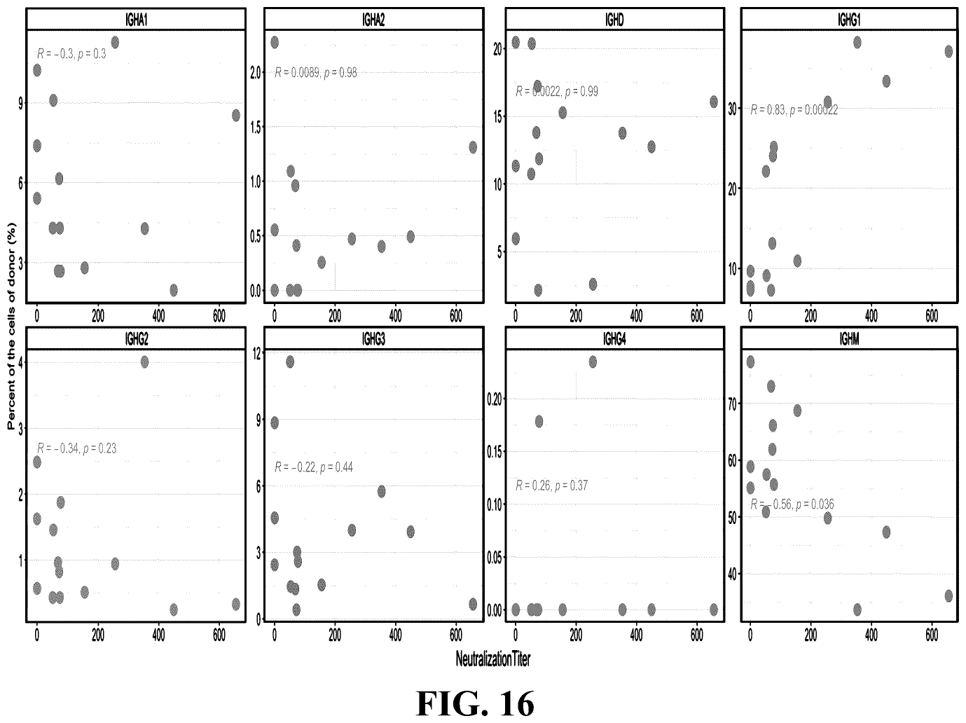

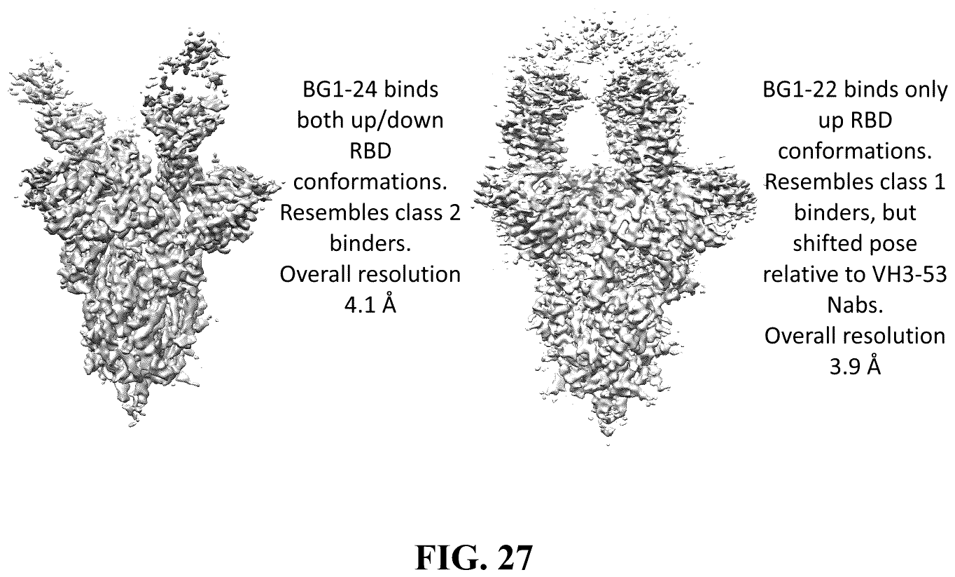

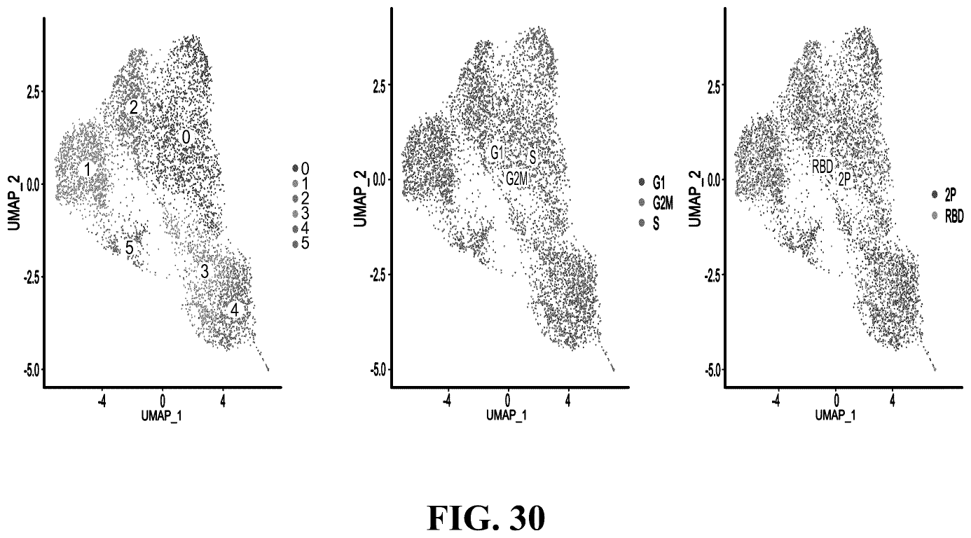

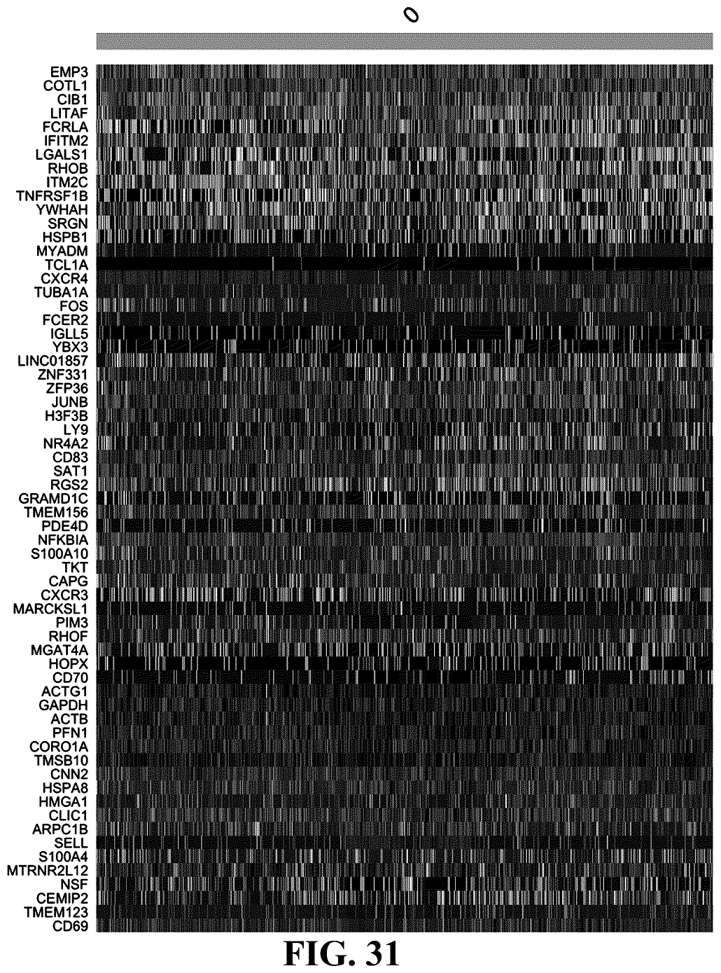

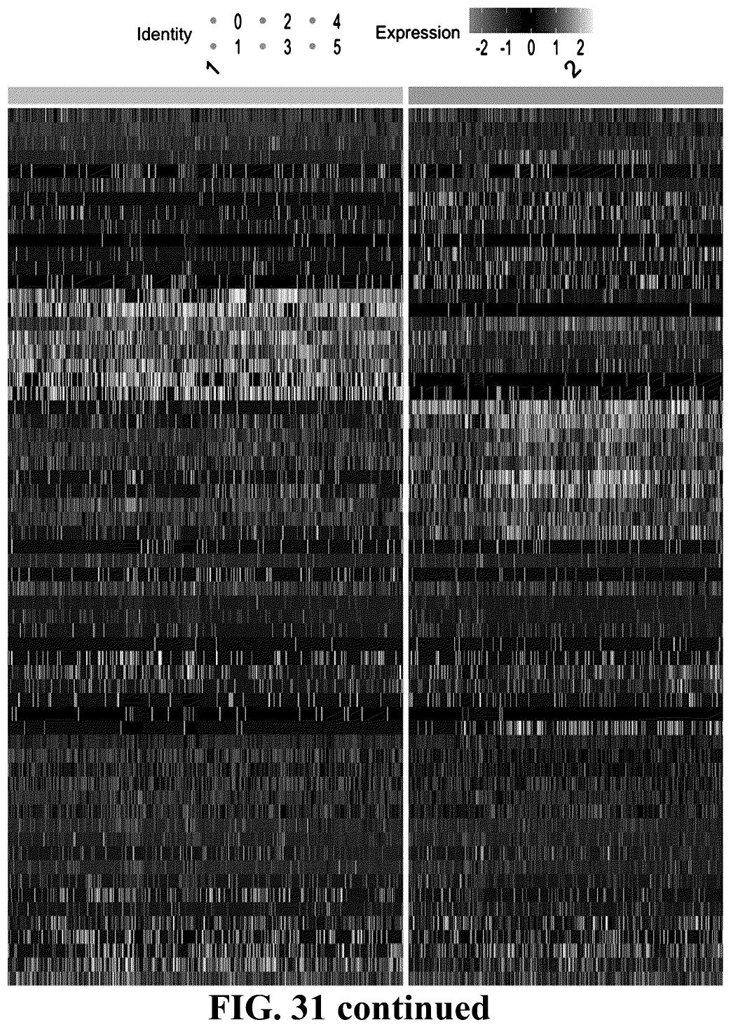

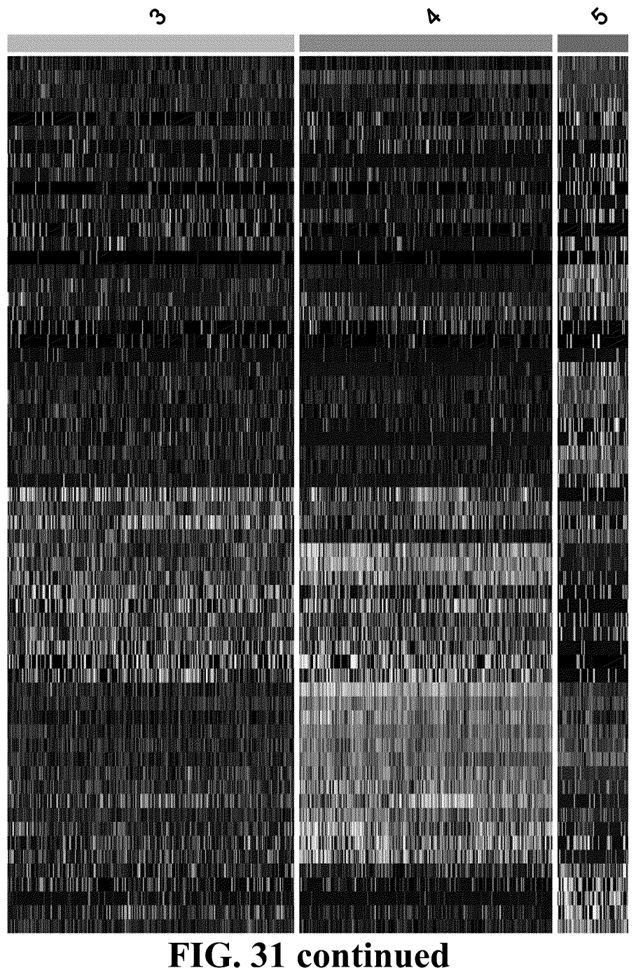

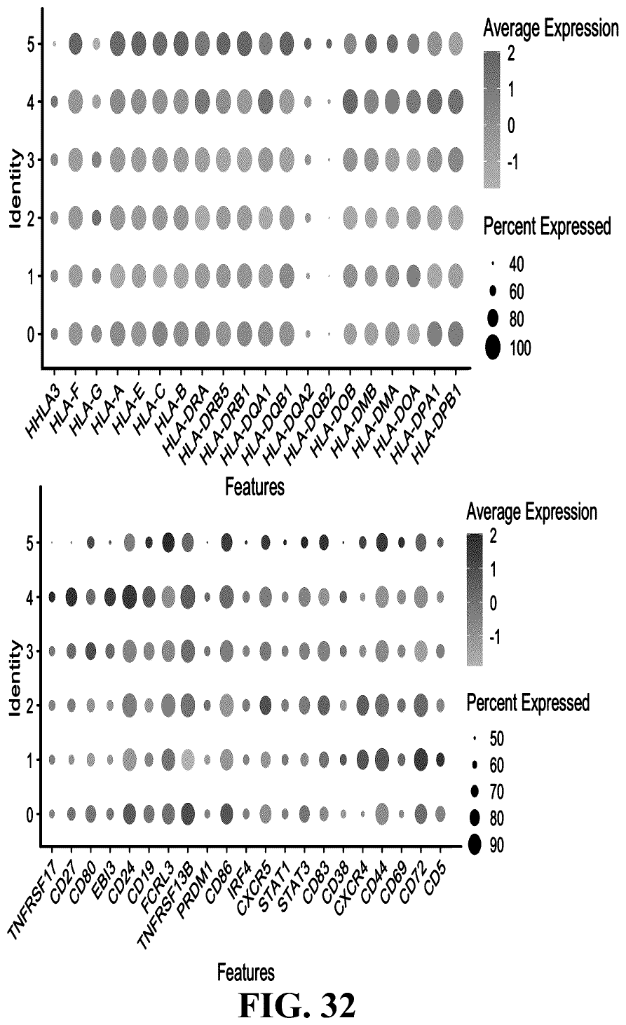

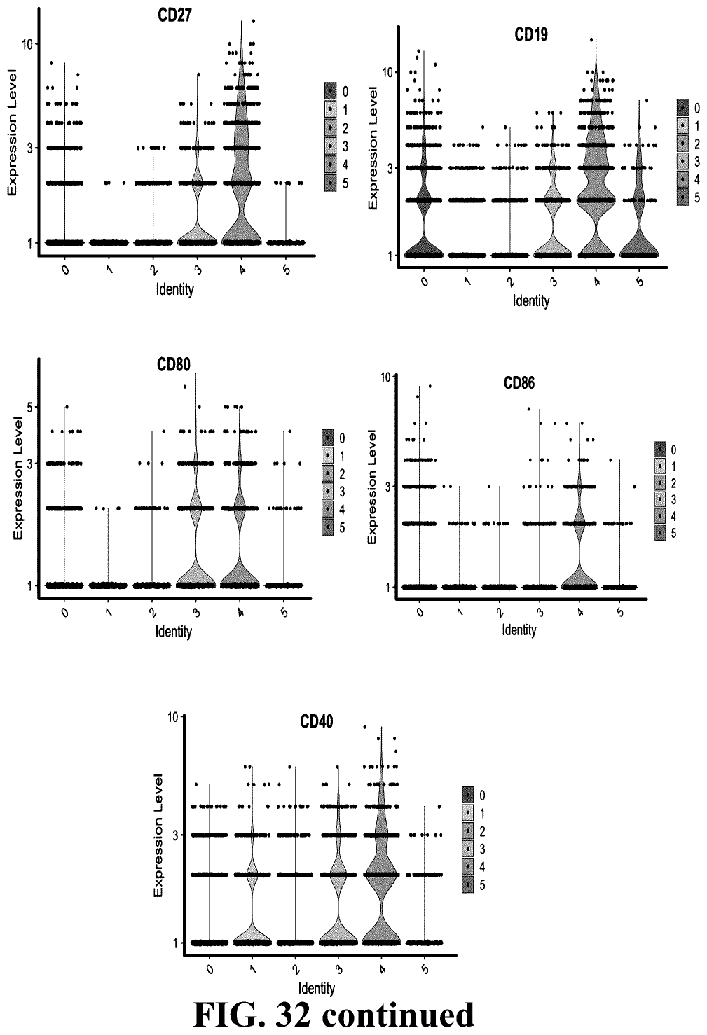

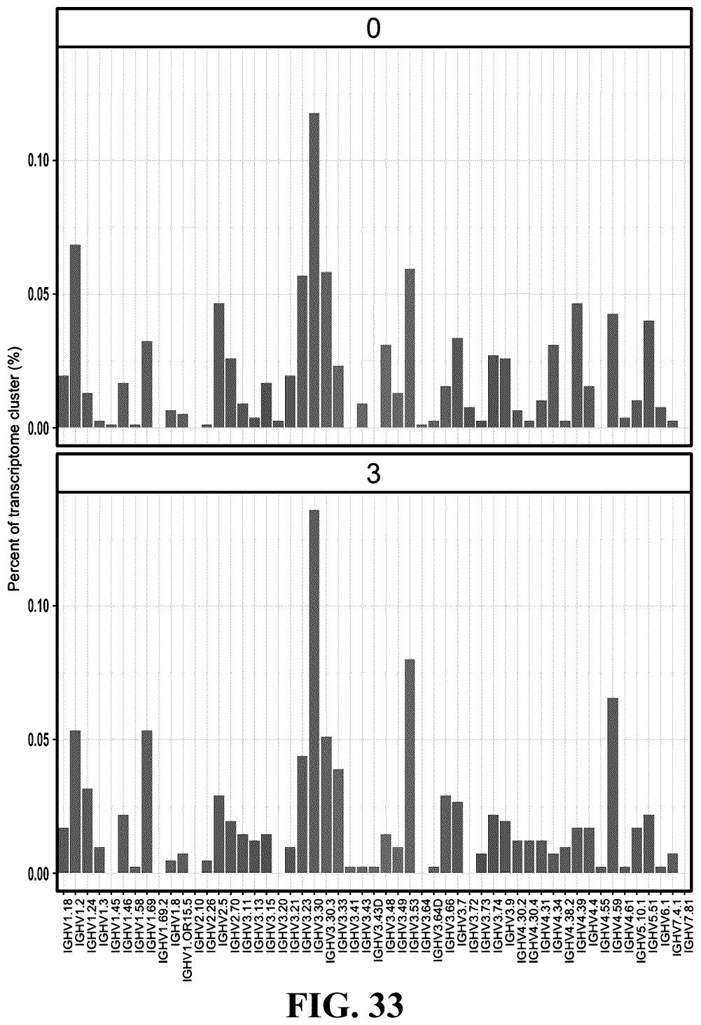

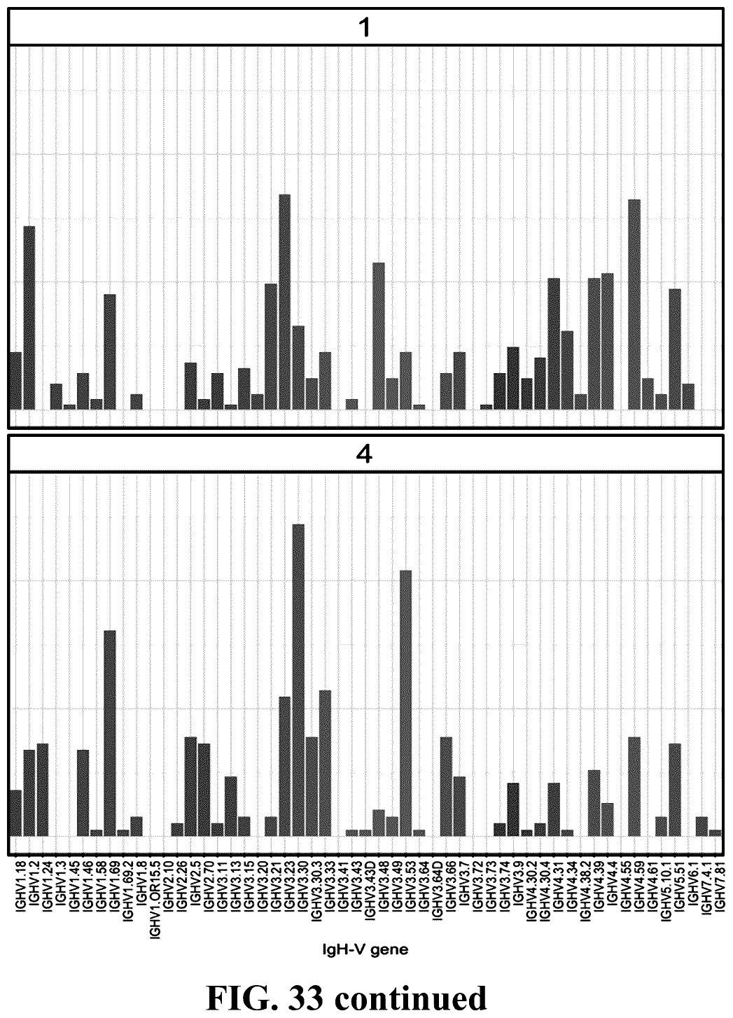

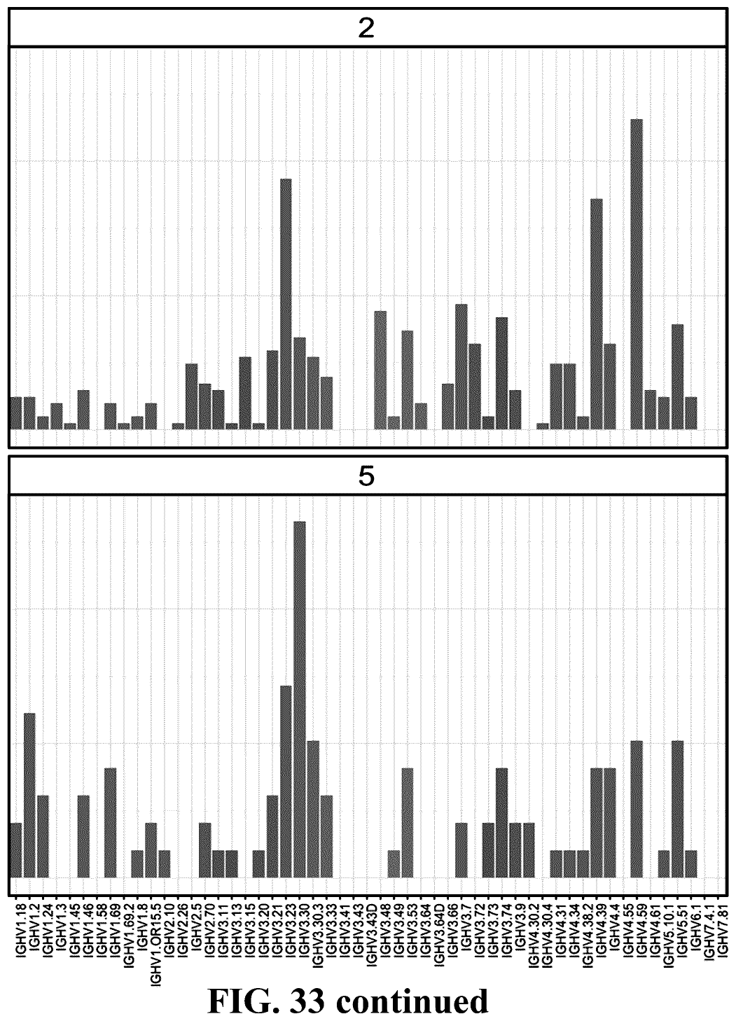

Although unliganded Fab structures often exhibit a disordered CDRH3 (e.g., 1RZI, 1RZF), it is unusual for an antibody bound to an antigen to exhibit a disordered CDRH3. From an examination of 731 antibody-antigen structures with resolutions of 3.5 Å or better in the Structural Antibody Database (SABDab; (Dunbar et al., 2014)), Applicants found only 6 with missing residue numbers between heavy chain residues 95 to 107, implying a disordered CDRH3 (PDB Codes 3LH2, 4JDT, 7JWB, 5ANM, 4M8Q, 3LHP). None of these complexes involved conventional antibody-antigen pairs; instead, they were germline forms of antibodies, the epitope was presented in a scaffold, or only the VH domain was involved in binding. This suggests that the orientation adopted by BG1-22 is not one that promotes CDRH3-mediated interactions with the antigen, as is classically observed in antibody-antigen structures, but instead simply accommodates the longer CDRH3 length by displacing much of the loop to outside the antibody-antigen interface. Taken together, these results provide further insight into the recurring VH3-53/VH3-66 neutralizing antibody class and suggests that longer CDRH3s, while infrequent, are not a restriction to V-gene mediated interactions at the RBD interface for VH3-53/VH3-66 antibodies. See, also, FIGS. 16, 27, 29 and 30-33.

TABLE 6
X-ray crystallographic data collection and refinement
statistics (related to FIGS. 5 and 13).

| | |
|---|---|
| | BG4-25 - SARS2-RBD |
| PDB ID | (12-2, SSRL) |

Data collection^{a}

| Space group | P2₁2₁2₁ | |
|---|---|---|
| Unit cell (Å) | 56.0, 104.3, 268.8 | |
| α, β, γ (°) | 90, 90, 90 | |
| Wavelength (Å) | 1.0 | |
| Resolution (Å) | 38.8-3.0 | (3.16-3.0) |
| Unique Reflections | 31,903 | (4079) |
| Completeness (%) | 97.9 | (88.1) |
| Redundancy | 6.1 | (4.2) |
| CC_{1/2} (%) | 97.2 | (25.9) |
| <I/σI> | 4.0 | (0.7) |
| Mosaicity (°) | 0.23 | |
| R_{merge} (%) | 27.1 | (135.8) |
| Rₚᵢₘ (%) | 12.5 | (88.6) |

| | |
|---|---|
| Wilson B-factor | 60.9 |

Refinement and Validation

| Resolution (Å) | 38.8-3.0 |
|---|---|
| Number of atoms | |
| Protein | 8,183 |
| Ligand | 0 |
| Waters | 0 |
| R_{work}/R_{free} (%) | 22.8/27.6 |
| R.m.s. deviations | |
| Bond lengths (Å) | 0.01 |
| Bond angles (°) | 1.6 |
| MolProbity score | 1.89 |
| Clashscore (all atom) | 18.9 |
| Poor rotamers (%) | 0.9 |
| Ramachandran plot | |

| | |
|---|---|
| Favored (%) | 86.4 |
| Allowed (%) | 11.1 |
| Disallowed (%) | 0.7 |
| Average B-factor (Å) | 80.5 |

^{a}Numbers in parentheses correspond to the highest resolution shell

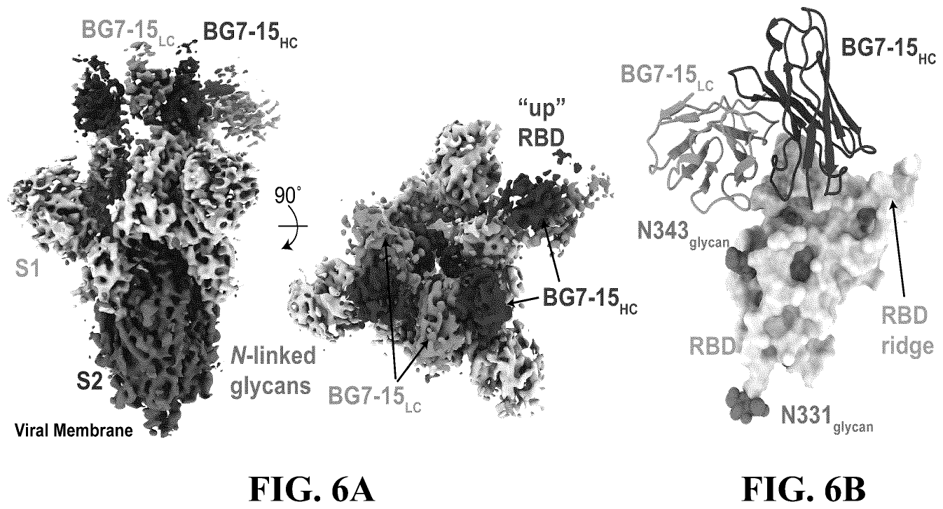

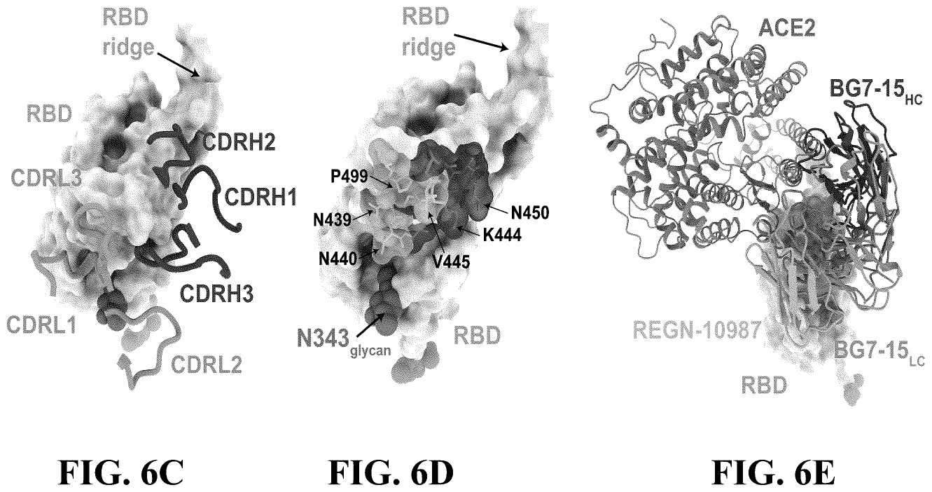

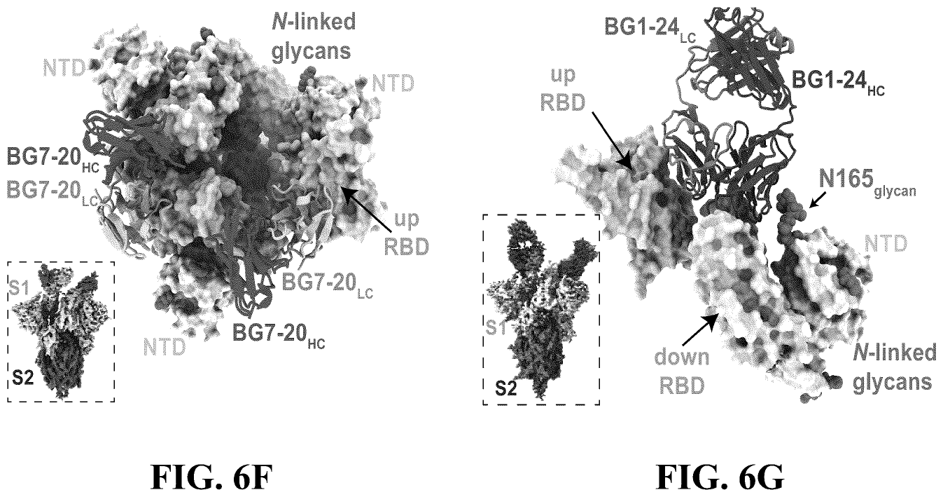

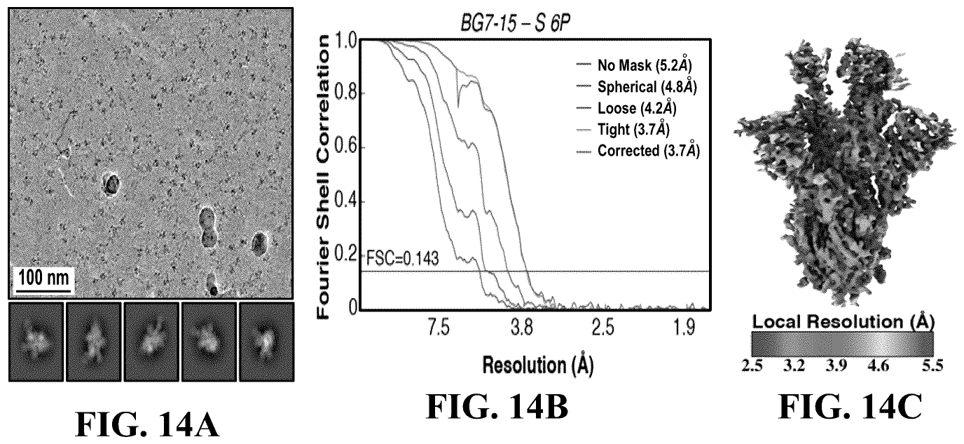

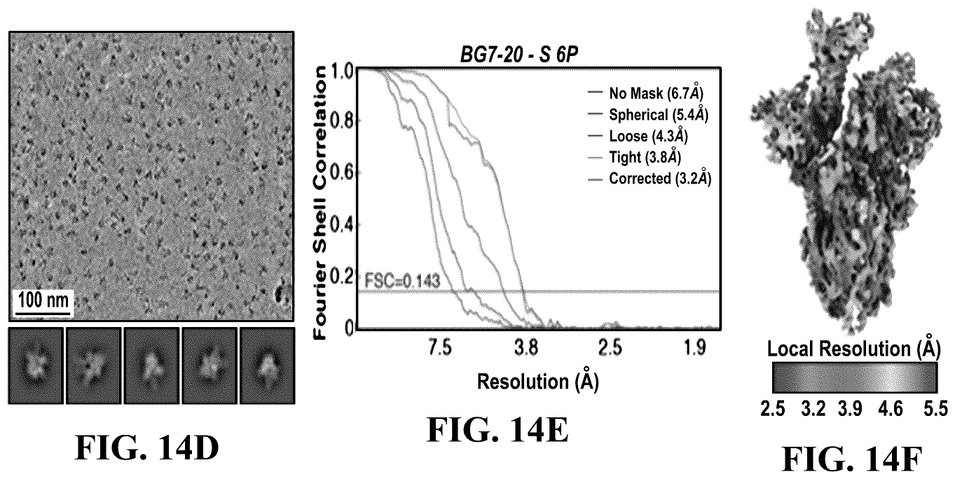

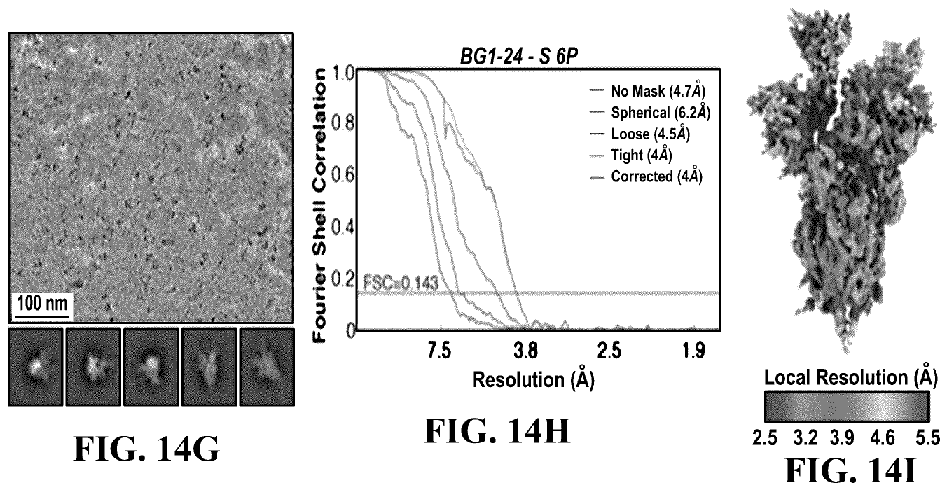

Structural characterization of neutralizing antibodies with distinct epitope recognition and mutational escape patterns. To further understand the specificity of RBD-targeting, Applicants determined cryo-EM structures of Fab-S complexes for three additional potently neutralizing antibodies: BG7-15 (VH1-18-encoded, 11 aa CDRH3, IC₈₀=92 ng/ml), BG7-20 (VH1-8-encoded, 20 aa CDRH3, IC₈₀=23 ng/ml) and BG1-24 (VH1-69-encoded, 16 aa CDRH3, IC₈₀=7 ng/ml), to resolutions of 3.7, 4.0, and 3.9 Å, respectively (FIGS. 6 and 14, Table 5). In all cases, Fabs were bound to either up or down RBD conformations, demonstrating recognition of antibody epitopes in either state. Analysis of the BG7-15-S structure revealed an antibody epitope focused on RBD residues 439-451, mainly mediated by contacts with CDRH3 and CDRL3 loops, which is non-overlapping with the ACE2 RBM (FIG. 6B-D). Overlay of BG7-15 and REGN-10987 (Hansen et al., 2020) showed a shared antibody footprint that binds outside the ACE2 RBM but would sterically hinder ACE2 receptor binding to RBD (FIG. 3H and FIG. 6E) while allowing binding of class 1 neutralizing antibodies (e.g., BG4-25, C102, REGN-10933).

Cryo-EM structures of BG7-20-S and BG1-24-S complexes revealed RBD-targeting similar to human neutralizing antibodies that belong to the class 2 binding mode (Barnes et al., 2020a). This class of SARS-COV-2 neutralizing antibodies is defined by potent neutralizers capable of recognizing up and down RBD conformations, an epitope that overlaps with the ACE2 RBM, has secondary interactions with neighboring “up” RBDs, and has the potential for intra-protomer avidity effects. Despite preferred VH gene usages yet to be established for class 2 antibodies (BG7-20 and BG1-24 are VH1-8 and VH1-69, respectively), BG7-20 and BG1-24 show a similar epitope focused along the RBD ridge that overlaps with residues involved in ACE2 binding and includes contacts with E484, F486, and Q493. The binding pose of BG1-24 promotes stabilization of the N165_{NTD} glycan, adding to the observation that class 2 antibodies can involve interprotomer glycan contacts (Cao et al., 2020a). Interestingly, the N-glycan interaction is mediated by a hydrophobic Met-Phe sequence at the tip of CDRH2, a common feature of VH1-69 antibodies (Chen et al., 2019). This feature has been attributed to facilitating broad neutralization by antibodies against influenza and Hepatitis C (Guthmiller et al., 2020) (Chen et al., 2019) and likely explains BG1-24's polyreactivity (FIG. 9E and Table 3).

With the use of monoclonal antibodies as therapeutic options for SARS-COV-2 infection, understanding possible RBD mutations selected under monoclonal antibody pressure and the frequency of SARS-COV-2 isolates harboring RBD mutations that confer immune escape is critical. While deep mutational scanning and in vitro selection experiments have facilitated the choice of therapeutic antibody cocktails, these experiments have also illustrated that single-point mutations are sufficient for viral escape (Greaney et al., 2020; Weisblum et al., 2020). Indeed, reports of the SARS-COV-2 spike variant N439K, which now accounts for 3-4% of global isolates, could potentially limit the use of REGN-10987 as a therapy in SARS-COV-2 infection (Barnes et al., 2020b).

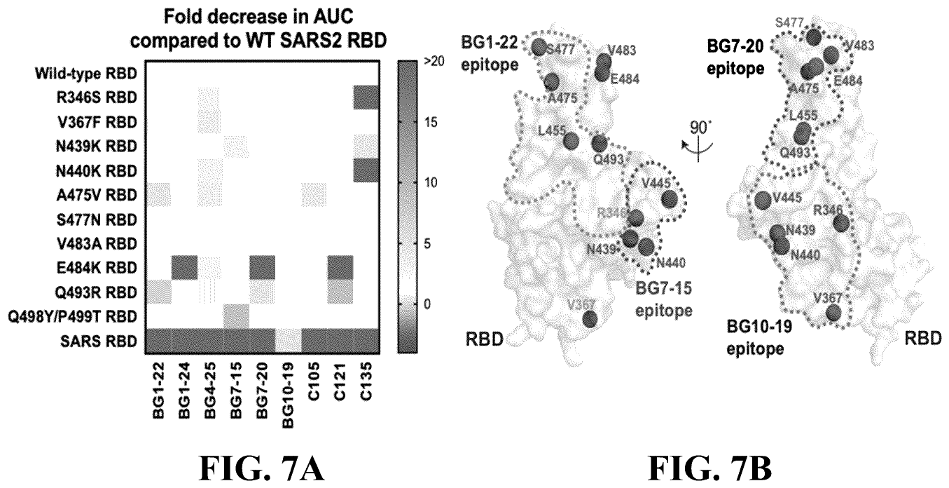

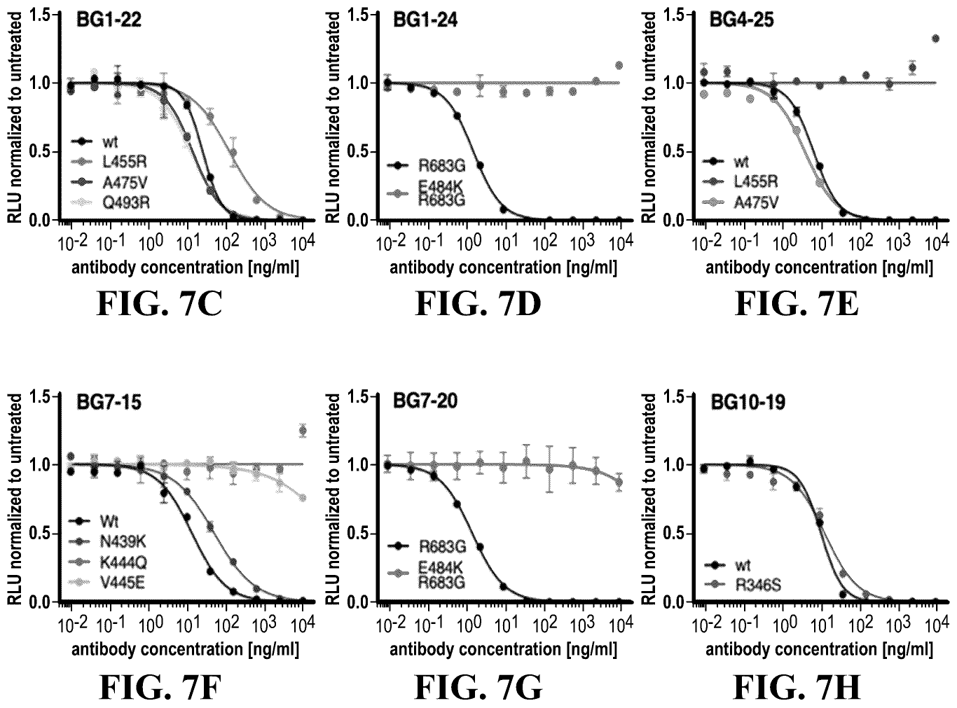

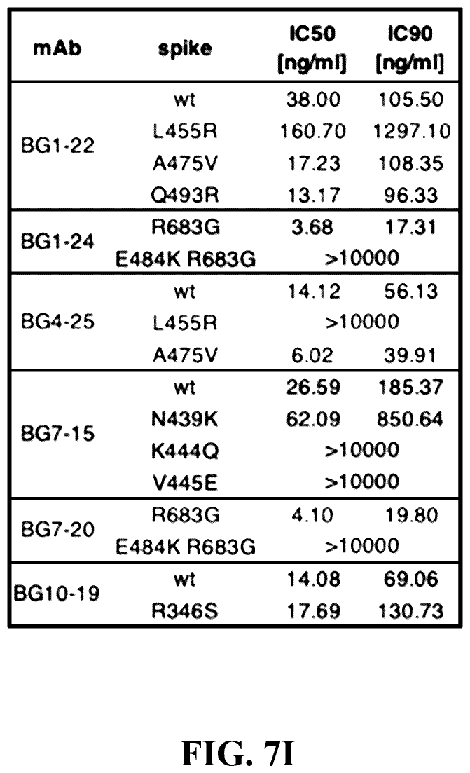

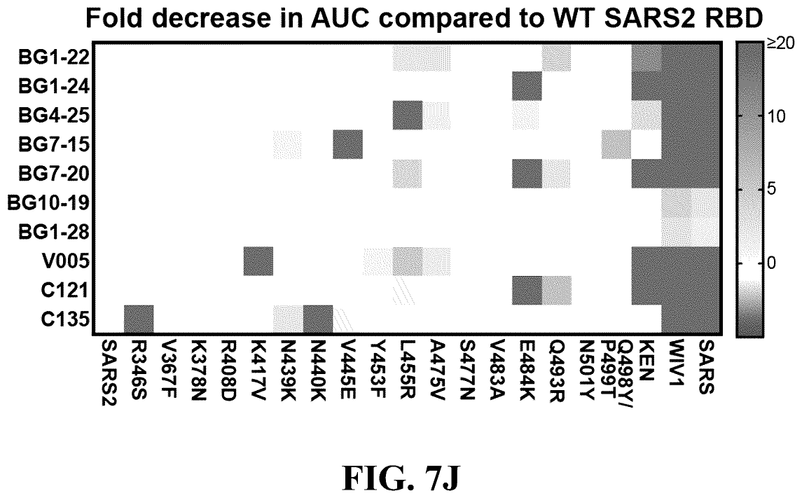

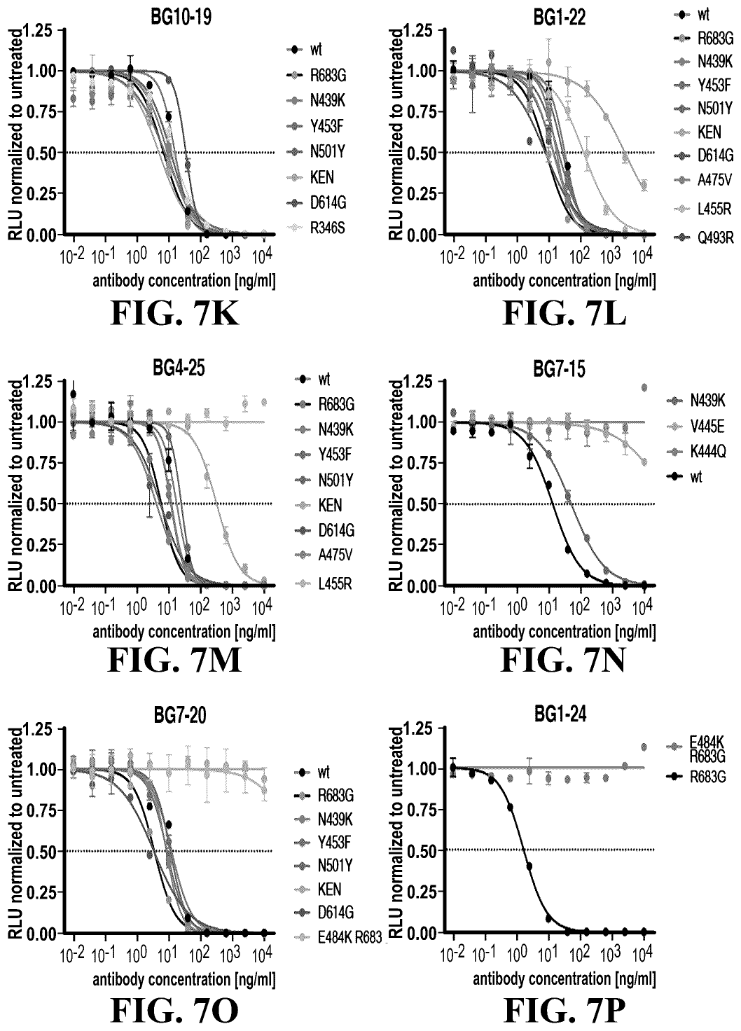

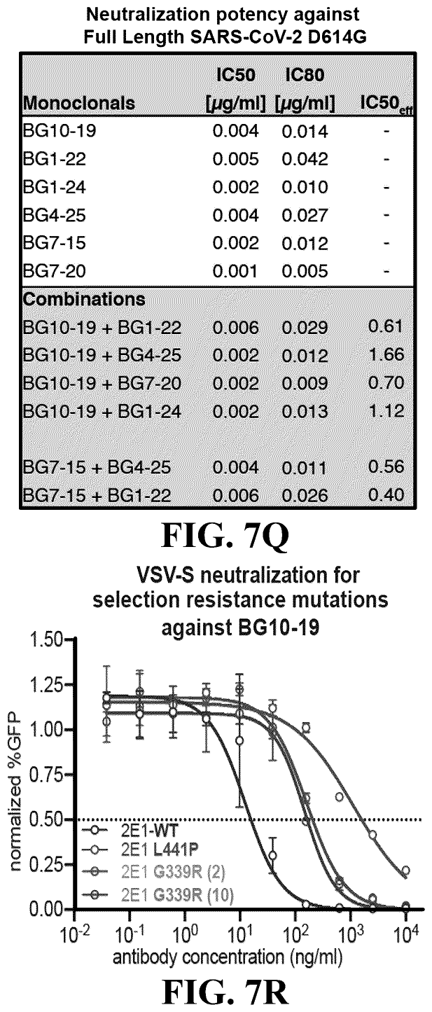

To assess the effects of RBD substitutions, Applicants assayed ELISA binding and SARS-COV-2 pseudovirus neutralization for 6 of the 7 antibodies reported in this study against a panel of RBD point mutations by ELISA (FIG. 7A-P). Decreased binding and neutralization potency was observed for most antibodies when RBD substitutions occurred within the antibody epitope and were consistent with RBD substitutions known to decrease binding of antibodies in the same class (FIG. 7C-Q) (Barnes et al., 2020a; Greaney et al., 2020). Notable exceptions included BG7-15, which showed neutralizing activity against the RBD N439K mutant that confers escape against REGN-10987, and BG4-25, which neutralized the class 1 escape variant A475V (FIG. 7M, N). In general, decreased binding affinity correlated with RBD mutations that conferred viral escape in pseudotyped viral neutralization assays and were consistent with the observation that RBD mutations that affect antibodies from one class do not affect antibodies in a different RBD-targeting class (FIGS. 7C-H and K-P). Given that several potently neutralizing antibodies were non-competitive, Applicants assessed antibody cocktails for synergistic neutralization effects (IC50_{eff}>1) against SARS-COV-2 pseudovirus using a previously described synergistic model (West et al., 2010). Results suggest that only the BG10-19+ BG4-25 antibody combination demonstrated synergistic neutralizing activity (FIG. 7Q) with other pairs showing no synergistic effects, consistent with published results for similarly classed antibody pairs (Hansen et al., 2020).

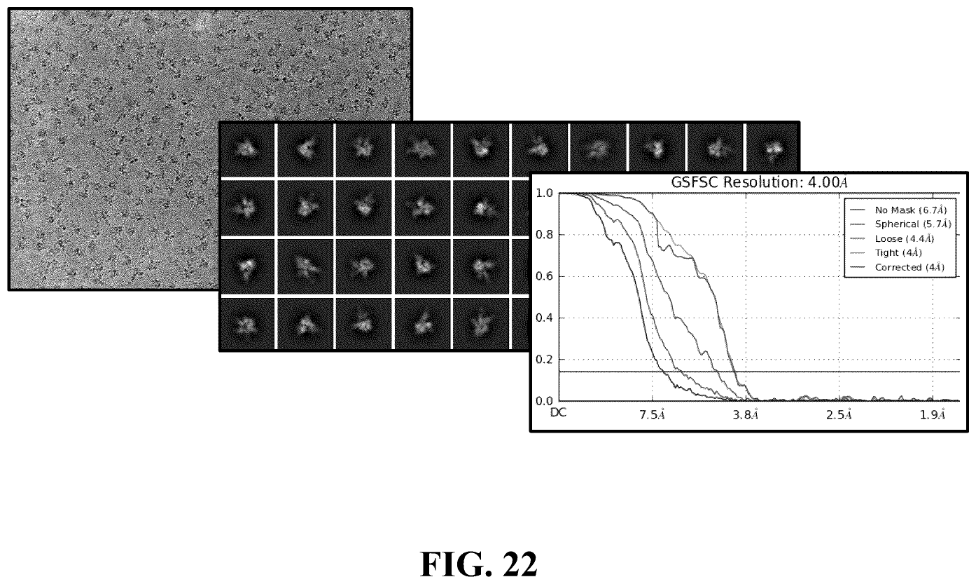

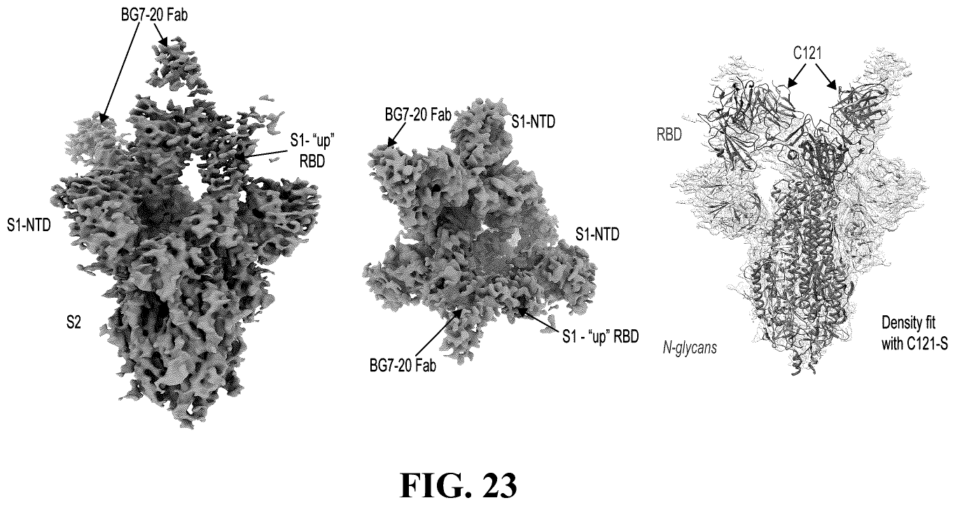

Finally, given the broad binding and neutralization activity of BG10-19 against a panel of SARS-COV-2 RBD substitutions (FIG. 7J, K), Applicants utilized a rVSV/SARS-COV-2 chimeric virus to determine spike variants that escape neutralization BG10-19 ((Weisblum et al., 2020) Methods). Initial attempts at selection experiments for BG10-19 showed no viral escape variants, consistent with observations for potently neutralizing convalescent plasma, antibody cocktails, and some monoclonal antibodies (Weisblum et al., 2020). However, after several passages, plaque purified viruses harboring the G339R and L441P mutations showed partial escape from BG10-19 (FIG. 7R). Thus, Applicants conclude that BG10-19's SARS-COV-2/SARS-CoV cross neutralization and ability to tolerate single RBD mutations within its epitope makes it an attractive therapeutic candidate as Applicants observe increasing point mutations among different SARS-COV-2 isolates (FIG. 10D). See, also, FIGS. 22, 23 and 27.

Discussion Antibodies play an indispensable role in antiviral immune responses both through their ability to neutralize viruses via interactions with their Fabs (Corti and Lanzavecchia, 2013) and by engaging other components of the immune system through interactions with their Fc regions (Bournazos et al., 2020). Characteristic features of different viruses influence the dynamics of how antiviral B cell and antibody responses are generated. HIV-1, for example, constrains efficient antibody responses through narrow structural pathways to broad neutralization (Scheid et al., 2011) and by causing B cell exhaustion (Moir et al., 2008).

In this study, Applicants show from a comprehensive in-depth analysis of SARS-COV-2 binding B cells from convalescent individuals 4-8 weeks post SARS-COV-2 infection that the repertoire of SARS-COV-2 binding B cells includes clonally expanded memory B cells and ABCs, as well as mature naïve B cells that are mostly observed as singlets, IgM⁺ or IgD⁺ and non-mutated. Interestingly, Applicants found clonal cells to be present in TC0, which is enriched in IgM⁺ B cells with inferred somatic mutations and low expression of CD27. Despite showing clonal expansion and somatic mutations, antibodies produced from this cluster were mostly low binding and non-neutralizing. An important point to consider in this context is that the comparatively low affinity of these antibodies could potentially be overcome in vivo if expressed as pentamers in the presence of J chain, similar to the observation of increased neutralization of some IgA antibodies when expressed as dimers. Applicants speculate that this cluster might contain cells from an early extrafollicular B cell response as also observed in influenza infection (Lam and Baumgarth, 2019). On the other hand, high binding and potent neutralizing activity were mostly detected in antibodies isolated from ABCs and memory B cells that shared transcriptional phenotypes across different individuals.

Immunologic correlates for protection from SARS-COV-2 after vaccination or prior exposure are not yet defined, but studies of other respiratory viruses suggest that serum neutralization could play an important role in protective immunity against SARS-COV-2 (Kulkarni et al., 2018; Verschoor et al., 2015). Consistent with other SARS-COV-2 studies, Applicants did not detect any intra-donor correlation between serum neutralization and the potency of monoclonal antibodies (Robbiani et al., 2020), but Applicants found a strong correlation between serum neutralization and the relative size of the ABC and memory B cell populations. This underscores the close relationship between high affinity memory B cell responses and serum antibody activity in SARS-COV-2. It is important to investigate if a similar correlation exists in individuals who have been vaccinated against SARS-COV-2 in an effort to delineate different responses to SARS-CoV-2 vaccines. Given its limited size, entry into the memory B cell compartment is tightly regulated by selection for high affinity of the immunoglobulin receptor (Victora and Nussenzweig, 2012). If neutralizing activity can also be selected for in the germinal center reaction is not known. Applicants did not find that to be the case, as both non-neutralizing high binders and neutralizers were found in TC3 and TC4 which are consistent with post germinal center B cells. This highlights the importance of integrating antibody structural and functional information in antiviral vaccine design, as neutralization cannot be independently selected for in the immune response (Chen et al., 2018).

The structural analysis revealed new insights into commonalities and differences among RBD-specific monoclonal antibodies. For example, in common with potently neutralizing antibodies C144 (Barnes et al., 2020a) and S2M11 (Tortorici, 2020), BG10-19 bridges between adjacent RBDs to lock the S trimer into a closed, prefusion conformation. However, in contrast to most previously described antibodies, BG10-19 recognizes a conserved epitope within the RBD core that is accessible in up/down RBD conformations on the spike trimer of both SARS-COV-2 and SARS-COV (unlike the conserved, cryptic CR3022 epitope only accessible on up RBD conformations), which may allow design of immunogens that elicit cross-reactive protection against future emerging coronaviruses. BG10-19's unique binding/neutralization properties and resistance to all RBD mutations identified in circulating isolates with a frequency >0.01%, including those found within the B.1.1.7 and B.1.351 lineages, make this an ideal therapeutic candidate in the arsenal against SARS-COV-2.

Additionally, high-resolution structures of VH3-53/VH3-66-class antibodies provided further understanding of the rules that govern potent neutralization and viral escape from this recurring antibody class and showed that CDRH3 length may not be a limitation to VH gene segment-mediated interactions at the RBD interface. Collectively, these structures and insights into the cellular processes behind the induction of potent, cross-reactive, neutralizing antibodies will not only aid in the battle to control the current COVID-19 pandemic through the use of safe and effective antibody treatments but will also provide additional criteria for the evaluation of humoral immune responses elicited from candidate vaccines against emerging zoonotic viruses with pandemic potential.

### Example 2-SARS/COV2/MERS Cross Binding Antibodies

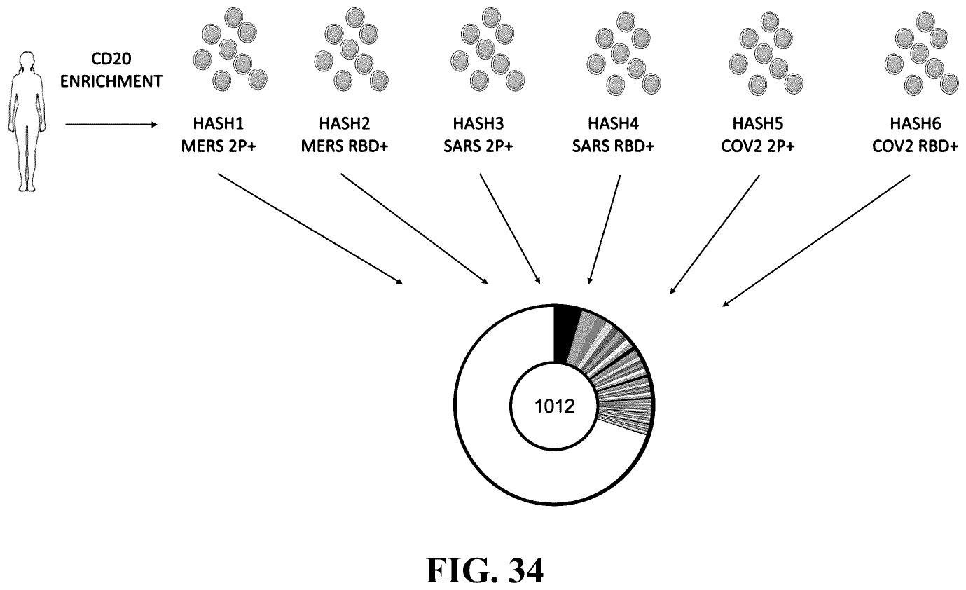

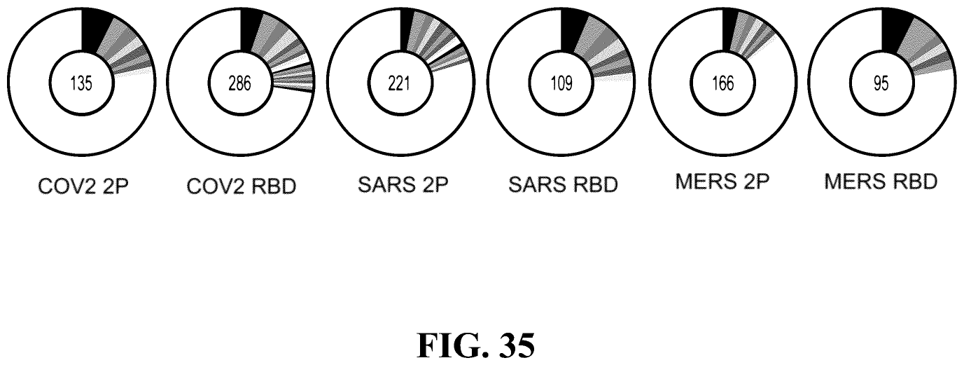

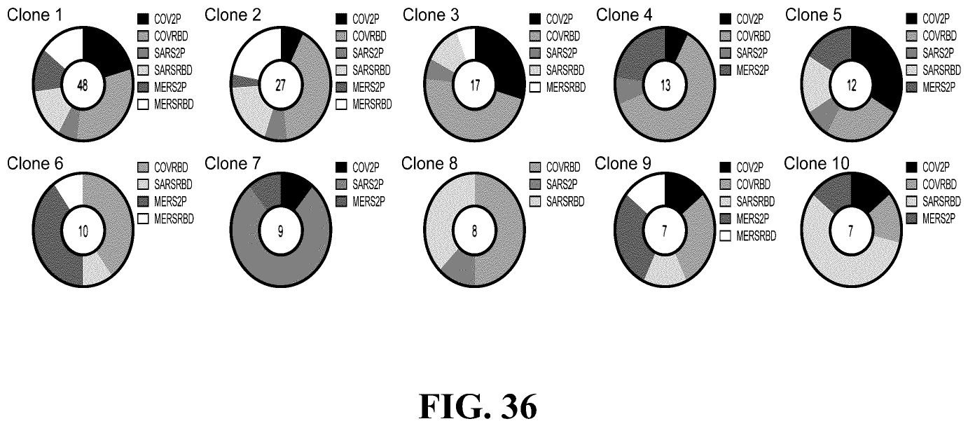

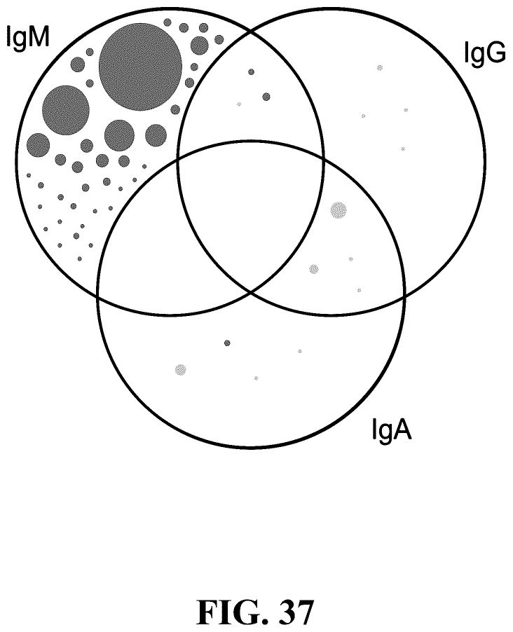

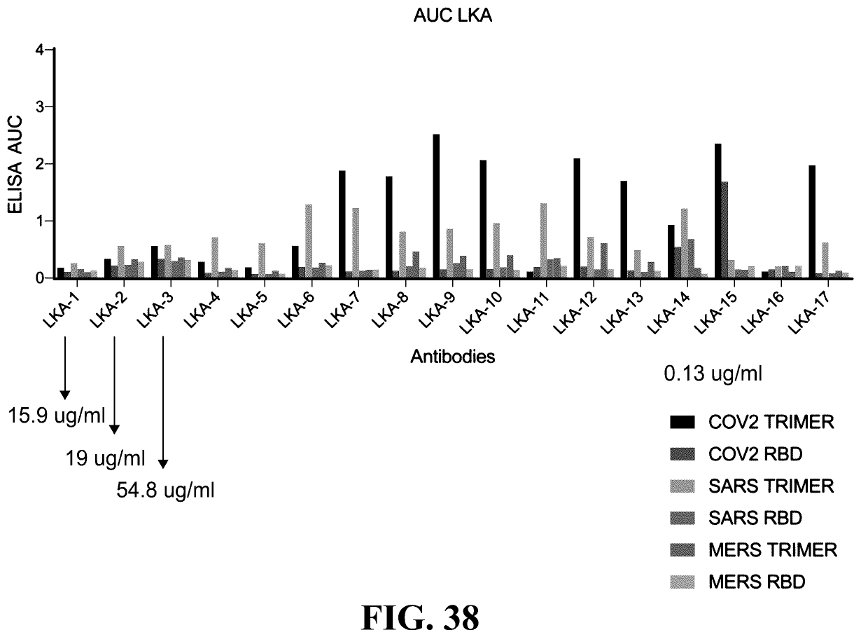

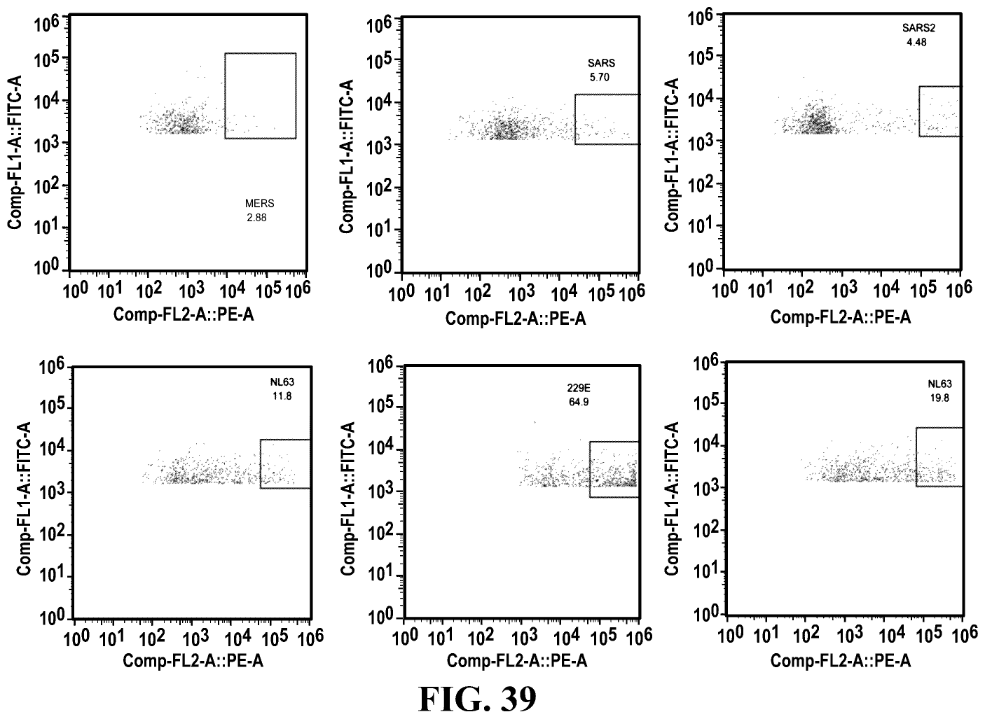

Applicants also identified antibodies that bind to SARS, COV2 and MERS (FIGS. 34-39 and Table 7). Applicants used Hash tags for cells specific to MERS, SARS and COV2 and sequenced the antibodies together (FIG. 34). When used as sorting bait all 6 proteins yielded differently expanded B cell clones (FIG. 35). There was considerable clonal overlap between B cells sorted with the 6 different proteins (FIG. 36). The majority of expanded clones were IgM+, but some expanded clones include class switched B cells (FIG. 37). Sorting with SARS/MERS/COV2 trimers selects for antibodies that bind outside of the RBD region of the spike protein (FIG. 38). Thus, the antibodies may be specific to non-mutated virus as most mutations occur in the RBD domain. Thus, the antibodies may be used for identifying immunogenic epitopes outside of the RBD domain. FIG. 39 shows staining with multiple coronavirus trimers for use in simultaneous staining.

TABLE 7
SARS/MERS/CoV2 Antibodies (SEQ ID NOS: 369-446).

| Iso- | VH | Heavy | CDR3 | Light | VK/VL | | CDR3 | | |
|---|---|---|---|---|---|---|---|---|---|
| type | Gene | Chain | Heavy Chain | Chain | Gene | | Light Chain | Virus | Name |
| IGHGI | IGHV4- | IGHJ6 | CARHWRDCSGGVC | IGL | IGLVI-40 | IGLJ2, | CQSYDGSL | MERS | LKA1 |
| | 59 | | QASQYSSMDVW | | | IGLJ3, | SESAVF | 2P | |
| | | | | | | IGLJ3 | | | |
| IGHGI | IGHV4- | IGHJ6 | CARHWRDCTGGVC | IGL | IGLVI-40 | IGLJ2, | CHSYDGSL | SARS | LKA2 |
| | 59 | | RASQYSSMDVW | | | IGLJ3, | SESAVF | 2P | |
| | | | | | | IGLJ3 | | | |
| IGHA2 | IGHV4- | IGHJ6 | CARHWRDCSGGVC | IGL | IGLVI-40 | IGLJ2, | CQSYDGSL | SARS | |
| | 59 | | RASQYSSMDVW | | | IGLJ3, | SESAVF | 2P | |
| | | | | | | IGLJ3 | | | |
| IGHA2 | IGHV4- | IGHJ6 | CARHWRDCSGGVC | IGL | IGLVI-40 | IGLJ2, | CQSYDGSL | SARS | |
| | 59 | | RASQYSSMDVW | | | IGLJ3, | SESAVF | 2P | |
| | | | | | | IGLJ3 | | | |
| IGHA1 | IGHV4- | IGHJ6 | CARHWRDCNRGVC | IGL | IGLVI-40 | IGLJ2, | CHSYDGTL | SARS | |
| | 59 | | RASQYSSMDVW | | | IGLJ3, | SESAVF | 2P | |
| | | | | | | IGLJ3 | | | |
| IGHGI | IGHV4- | IGHJ6 | CARHWRECSGGVC | IGL | IGLVI-40 | IGLJ2, | CQSYDGSL | SARS | |
| | 59 | | RASQYSSMDVW | | | IGLJ3, | SESAVF | 2P | |
| | | | | | | IGLJ3 | | | |
| IGHGI | IGHV4- | IGHJ6 | CARHWRDCNGGVC | IGL | IGLVI-40 | IGLJ2, | CQSYDGSL | SARS | |
| | 59 | | RASQYASMDVW | | | IGLJ3, | SESAVF | 2P | |
| | | | | | | IGLJ3 | | | |
| IGHGI | IGHV4- | IGHJ6 | CARHWRECSGGVC | IGL | IGLVI-40 | IGLJ2, | CQSYDGSL | SARS | |
| | 59 | | RTSQYSSMDVW | | | IGLJ3, | SESAVF | 2P | |
| | | | | | | IGLJ3 | | | |
| IGHGI | IGHV4- | IGHJ6 | CARHWRDCSGGVC | IGL | IGLVI-40 | IGLJ3 | CHSYDGSL | COVID | LKA3 |
| | 59 | | RASQYSAMDVW | | | | SESAVF | 2P | |
| IGHA1 | IGHV3- | IGHJ3 | CAHVRPLTGYHVGH | IGL | GLVI-51 | IGLJ2, | CGTWDTSL | MERS | LKA4 |
| | 30 | | DPFDIW | | | IGLJ3, | SIGVF | 2P | |
| | | | | | | IGLJ3 | | | |
| IGHA1 | IGHV3- | IGHJ3 | CAHVRPLTGYHVGH | IGL | IGLVI-51 | IGLJ2, | CGTWDTSL | MERS | |
| | 30 | | DPFDIW | | | IGLJ3, | RIGVF | 2P | |
| | | | | | | IGLJ3 | | | |
| IGHA1 | IGHV3- | IGHJ3 | CAHVRPLTGYHVGH | IGL | IGLVI-51 | IGLJ3 | CGTWDTSL | SARS | LKA5 |
| | 30 | | DPFDIW | | | | SIGVF | 2P | |
| IGHA1 | IGHV3- | IGHJ3 | CAHVRPLTGYHVGH | IGL | IGLVI-51 | IGLJ2, | CGTWDTSL | SARS | |
| | 30 | | DPFDIW | | | IGLJ3, | SAGVF | 2P | |
| | | | | | | IGLJ3 | | | |
| IGHA1 | IGHV3- | IGHJ3 | CAHVRPLTGYHVGR | IGL | IGLVI-51 | IGLJ2, | CGTWDTSL | SARS | |
| | 30 | | DPFDIW | | | IGLJ3, | SAGVF | 2P | |
| | | | | | | IGLJ3 | | | |
| IGHA1 | IGHV3- | IGHJ3 | CAHVRPLTGYHVGR | IGL | IGLV3-1 | IGLJ2, | CQAWNN | SARS | |
| | 30 | | DPFDVW | | | IGLJ3 | NNVVF | 2P | |
| IGHA1 | IGHV4- | IGHJ4 | CARGFDYW | IGK | IGKV3- | IGKJI | CQQYGNS | MERS | LKA6 |
| | 59 | | | | 20 | | PWTF | 2P | |
| IGHGI | IGHV4- | IGHJ4 | CARGFDYW | IGK | IGKV3- | IGKJI | CLQYGGSG | SARS | LKA7 |
| | 59 | | | | 20 | | WTF | 2P | |
| IGHGI | IGHV4- | IGHJ4 | CARGFDYW | IGK | IGKV3- | IGKJI | CQQYGSSP | SARS | |
| | 59 | | | | 20 | | WTF | 2P | |
| IGHG3 | IGHV4- | IGHJ4 | CARGFDYW | IGK | IGKV3- | IGKJI | CQQYGSSP | SARS | |
| | 59 | | | | 20 | | WTF | 2P | |
| IGHGI | IGHV4- | IGHJ4 | CARGFDYW | IGK | IGKV3- | IGKJI | CHQYGSSP | SARS | |
| | 59 | | | | 20 | | WTF | 2P | |
| IGHGI | IGHV4- | IGHJ3 | CARRIYGGDGFDIW | IGL | IGKV3- | IGLJI | CQVWHGT | SARS | LKA8 |
| | 59 | | | | 21 | | GDQYVF | 2P | |
| IGHGI | IGHV4- | IGHJ3 | CARRIYGGDGFDIW | IGL | IGKV3- | IGLJI | CQVWPGS | SARS | |
| | 59 | | | | 21 | | SDHYVF | 2P | |
| IGHGI | IGHV4- | IGHJ3 | CARRIYGGDGFDIW | IGL | IGKV3- | IGLJI | CQVWHGT | SARS | |
| | 59 | | | | 21 | | SDHYVF | 2P | |
| IGHA1 | IGHV3- | IGHJ6 | CARSRGGSYYYGM | IGK | IGKV1- | IGKJ2, | CQQSYSTL | SARS | LKA9 |
| | 30 | | DVW | | 39 | IGKJ2 | ETF | 2P | |
| IGHGI | IGHV3- | IGHJ6 | CARSRGGSYYYGM | IGK | IGKV1- | IGKJI | CQHYNSFS | SARS | |
| | 30 | | DVW | | 5 | | PTWTF | 2P | |
| IGHGI | IGHV3- | IGHJ6 | CARAAGGGYYYGM | IGL | IGLV2- | IGLJI | CCSYAGGY | SARS | LKA10 |
| | 30 | | DVW | | 23 | | VF | 2P | |
| IGHGI | IGHV3- | IGHJ6 | CARAAGGNYYYGM | IGK | IGKV1- | IGKJ4 | CQQSYGSP | SARS | |
| | 30 | | DVW | | 39 | | LTF | 2P | |
| IGHA1 | IGHV4- | IGHJ3 | CARSVAALTWELNK | IGK | IGKV3- | IGKJI | CQQYGSPT | COVID | LKA11 |
| | 59 | | LAFDVW | | 20 | | WTF | 2P | |
| IGHA1 | IGHV4- | IGHJ3 | CARSVAALTWELNK | IGK | IGKV3- | IGKJI | CQQYGSPT | COVID | |
| | 59 | | LAFDVW | | 20 | | WTF | RBD | |
| IGHA1 | IGHV1- | IGHJ4 | CAQTTPYDSSGYYRS | IGK | IGKV3- | IGKJ4 | CQQRTNW | MERS | LKA12 |
| | 69 | | YW | | 11 | | PPRLTF | 2P | |
| IGHA1 | IGHV1- | IGHJ4 | CAETTPYDSSGYYRS | IGK | IGKV3- | IGKJ4 | CQQRTNW | SARS | LKA13 |
| | 69 | | YW | | 11 | | PPRLTF | 2P | |
| IGHGI | IGHVS- | IGHJ4 | CARWEAGGTSPLDY | IGK | IGKVI-5 | IGKJ2 | CQRYNSYS | SARS | LKA14 |
| | 51 | | W | | | | QNTF | 2P | |
| IGHGI | IGHVS- | IGHJ4 | CARWEAGGTSPLDS | IGK | IGKVI-5 | IGKJ2, | CQQYSSYS | SARS | |
| | 51 | | W | | | IGKJ2 | QITF | 2P | |
| IGHA1 | IGHV3- | IGHJ4 | CARGYGDYYFDYW | IGK | IGKV3- | IGKJ2, | CQHFGSSP | SARS | LKA15 |
| | 66 | | | | 20 | IGKJ2, | LF | 2P | |
| | | | | | | IGKJ2 | | | |
| IGHGI | IGHV3- | IGHJ4 | CARAYGDYYFDYW | IGK | IGKV3- | IGKJ2, | CQQYRTF | SARS | |
| | 66 | | | | 20 | IGKJ2 | | 2P | |
| IGHG3 | IGHVS- | IGHJ5 | CARHPKGYGLRLPN | IGL | IGLV2- | IGLJI | CSSYTSSST | SARS | LKA16 |
| | 51 | | WFDPW | | 14 | | FF | 2P | |
| IGHG3 | IGHVS- | IGHJ5 | CARHPKGYGLRLPN | IGL | IGLV2- | IGLJI | CSSYTTSST | SARS | |
| | 51 | | WFDPW | | 14 | | FF | 2P | |
| IGHGI | IGHV3- | IGHJ4 | CAKDLLPYRCSGGS | IGL | IGLVI- | IGLJ2, | CSAWDDS | SARS | LKA17 |
| | 23 | | CFGLYFDYW | | 44 | IGLJ3 | LNGVVF | 2P | |
| IGHM | IGHV3- | IGHJ4 | CAKDLLPYRCSGGS | IGL | IGLVI- | IGLJ2, | CAAWDDS | SARS | |
| | 23 | | CFGFYFDYW | | 44 | IGLJ3 | LNGVVF | 2P | |

### Example 3—Methods

Experimental model and subject details. All work with human samples was performed in accordance with approved Institutional Review Board protocols (IRB) which were reviewed by the IRB at Brigham and Women's Hospital, Boston. Subjects who had recovered from COVID-19 (Table 1) were recruited through a patient cohort that has been created in collaboration between The Broad Institute of MIT and Harvard, Cambridge (MA, USA) and Brigham and Women's Hospital, Boston (MA, USA) under IRB protocol 2020P000849, “Biorepository for Samples from those at increased risk for or infected with SARS-COV-2”. Blood draws were performed at Brigham and Women's Hospital.

Serum RBD Enzyme-linked immunosorbent assay (ELISA). Serum ELISAs against SARS-COV-2 RBD were performed in a protocol modified from (Roy et al., 2020). MaxiSorp 384-well microplates (Sigma) were coated with 50 μl/well of 2,500 ng/ml of SARS-COV-2 RBD in coating buffer (1 packet BupH carbonate-bicarbonate (ThermoFisher) in 500 ml of Milli-Q H20) overnight at 4° C. Plates were then washed 3 times with 100 μl/well of wash buffer (0.05% Tween-20, 400 mM NaCl, and 50 mM Tris-HCl [pH 8.0] in Milli-Q H20) using a BioTek 406 plate washer. Plates were blocked by adding 100 μl/well of blocking buffer (1% BSA, 140 mM NaCl, and 50 mM Tris-HCl (pH 8.0)) for 30 min at room temperature. Plates were then washed as described above. 50 μl of 1:100 diluted serum samples in dilution buffer (1% BSA, 0.05% Tween-20, 140 mM NaCl, and 50 mM Tris-HCl (pH 8.0)) were added to the wells and incubated for 30 min at 37° C. Plates were then washed 7 times as described above. 50 μl/well of 1:25,000 diluted detection antibody solution (HRP-anti human IgG and IgM, Bethyl Laboratory #A80-104P, A80-100P) was added to the wells and incubated for 30 min at room temperature. Plates were then washed 7 times as described above. 40 μl/well of Pierce TMB peroxidase substrate (ThermoFisher) was then added to the wells and incubated at room temperature for 3 min (IgG) or 5 min (IgM). The reaction was then stopped by adding 40 μl/well of stop solution (0.5M H2S04 in Milli-Q H20) to each well. The OD was read after 15 min at 450 nm and 570 nm on a BioTek Synergy HT. For control antibodies CR3022 (Tian et al., 2020) IgG1 and IgM (Absolute Antibody #Ab01680-10.0, Ab01680-15.0) dilution curves, the antibodies were diluted to a concentration of 1 μg/ml in dilution buffer and duplicate 12 two-fold serial dilution curves were generated. One known positive and two known negative samples were included on each plate as controls.

Serum ELISA analysis. A standard curve based on absorbances from the monoclonal antibody CR3022 (Tian et al., 2020) dilution series included with each plate was used to estimate antibody abundance in test samples and allow for comparison of results across batches. Estimated antibody abundance in test samples was compared to the background signal from a cohort of pre-pandemic serum samples that served as negative controls. Serum samples with antibody abundance greater than 3 standard deviations (SD) above the mean of the pre-pandemic serum samples were considered to be positive and samples with antibody abundance less than 3 SD above the mean of the pre-pandemic serum samples were considered negative.

SARS-COV-2 and SARS-COV pseudovirus neutralization assay. Neutralizing activity against SARS-COV-2 pseudovirus was measured using a single-round infection assay in human ACE2-expressing target cells. Convalescent patient serum samples were tested against pseudotyped virus particles produced in 293T/17 cells (American Type Culture Collection) by co-transfection of plasmids encoding codon-optimized S (containing D at position 614) with a partially deleted cytoplasmic tail (provided by Dr. Dan Barouch, Beth Israel Deaconess Medical Center), and the HIV-1 backbone vector SG3 Δ Env (NIH AIDS Reagent Program). This pseudovirus strain was used for infecting TZM.bl/ACE2 target cells which encode an integrated luciferase reporter gene under control of an HIV-1 LTR. Subsequently, a second pseudovirus assay platform was implemented and used for testing neutralizing activity of purified monoclonal antibodies. This assay platform utilized pseudovirus produced in 293T/17 cells by co-transfection of plasmids encoding codon-optimized SARS-COV-2 full-length S (containing G at position 614), packaging plasmid pCMV ΔR8.2 expressing HIV-1 gag and pol, and luciferase reporter plasmid pHR′ CMV-Luc. Plasmids were kindly provided by Dr. Barney Graham (NIH, Vaccine Research Center). This pseudovirus strain was used for infecting 293/ACE2 target cells. The 293T and TZM.bl cell lines stably overexpressing the human ACE2 cell surface receptor protein were kindly provided by Drs. Michael Farzan and Huihui Ma (The Scripps Research Institute). For neutralization assays, serial dilutions of patient serum samples (primary 1:20 with 3-fold dilution series) or antibodies (up to 50 μg/ml with 5-fold dilution series) were performed in duplicate followed by addition of pseudovirus. Plates were incubated for 1 hour at 37° C. followed by addition of 293T/ACE2 or TZM.bl/ACE2 target cells (1×10⁴/well). Wells containing cells+pseudovirus (without sample) or cells alone acted as positive and negative infection controls, respectively. Assays were harvested on either day 2 (TZM.bl/ACE2 target cells) or day 3 (293/ACE2 target cells) using Promega Bright-Glo luciferase reagent and luminescence detected with a Perkin-Elmer Victor luminometer. Titers were determined as the serum dilution or antibody concentration that inhibited 50% or 80% virus infection (serum ID₅₀/ID₈₀ or antibody IC₅₀/IC₈₀ titers, respectively).

Similarly, neutralizing activity of monoclonal antibodies against SARS-COV pseudovirus as well as SARS-COV-2 variants B.1.1.7 and B.1.351 was determined using HIV-based lentiviral particles pseudotyped with either SARS-COV S lacking the C-terminal 21 amino acids of the cytoplasmic tail or SARS-COV-2 S (containing G at position 614) carrying the reported spike mutations in B.1.1.7 and B.1.351 (Tegally et al., 2020) (Davies et al., 2021). Pseudotyped particles were generated and neutralization assays were performed as previously described (Crawford et al., 2020a) (Crawford et al., 2020b). Briefly, the genes encoding the respective spike proteins were co-transfected with Env-deficient HIV backbone to create pseudotyped lentiviral particles. For neutralization assays, 4- or 5-fold serially diluted purified IgG was incubated with SARS-COV pseudotyped virus for 1 hour at 37° C. The virus/antibody mixture was added to 293TACE2 target cells and incubated for 48 hours at 37° C., then cells were lysed and luciferase activity was measured using Britelite Plus (Perkin Elmer). Relative luminescence units (RLUs) were normalized to values derived from cells infected with pseudotyped virus in the absence of antibody. Data were fit to a 5-parameter nonlinear regression in AntibodyDatabase (West et al., 2013).

Blood sample processing. 60-80 ml of blood from each donor were processed using Ficoll Paque Plus (GE Healthcare) in order to isolate peripheral blood mononuclear cells (PBMCs) according to the manufacturer's instructions. After PBMC isolation Applicants immediately proceeded with isolation of CD20 B cells using magnetic cell separation (MACS). In brief, cells were resuspended in MACS buffer (phosphate buffered saline (PBS) pH 7.2, 0.5% bovine serum albumin (BSA) and 2 mM EDTA) and stained with mouse anti-human CD20 IgG1 antibody coupled to magnetic beads (Miltenyi Biotec, #130-091-104). After washing and resuspending in MACS buffer cells were added to LS columns (Miltenyi Biotec) placed on a magnetic stand and the columns washed 3 times with 3 ml MACS buffer before removing the columns from the magnet and elution of cells according to the manufacturer's instructions. Cells were then washed and resuspended in Cell Staining Buffer (Biolegend, #420201).

Cell staining and sorting. CD20 enriched cells were stained with DNA-barcoded Totalseq C antibodies (Biolegend) according to the manufacturer's instructions. In brief, cells underwent Fc receptor blocking with Human TruStain FcX™ Fc (Biolegend) for 10 minutes at 4° C., after which cells were washed and resuspended in Cell Staining Buffer (Biolegend, #420201). Cells from different subjects were then stained with different Totalseq C antibodies (Biolegend): 1 (TotalSeq™-C0251 anti-human Hashtag 1 Antibody), 2 (TotalSeq™-C0252 anti-human Hashtag 2 Antibody), 3 (TotalSeq™-C0253 anti-human Hashtag 3 Antibody), 4 (TotalSeq™-C0254 anti-human Hashtag 4 Antibody), 6 (TotalSeq™-C0256 anti-human Hashtag 6 Antibody), 7 (TotalSeq™-C0257 anti-human Hashtag 7 Antibody), 8 (TotalSeq™-C0258 anti-human Hashtag 8 Antibody), 10 (TotalSeq™-C0260 anti-human Hashtag 10 Antibody). After staining with hashing antibodies, cells were washed 3 times in Cell Staining Buffer (Biolegend, #420201) and then up to 7 separate samples were combined for staining of antigen binding B cells. For this, the combined samples were stained with either 1 μg/ml biotinylated SARS-COV-2 spike trimer or 1 μg/ml biotinylated SARS-COV-2 RBD (See below for protein expression; biotinylation was performed using avitag technology (Avidity) following the manufacturer's instructions). Cells were simultaneously stained with FITC mouse anti-human CD19 antibody (BD, 340864) and incubated for 20 minutes at 4° C. before they were washed and resuspended in PBS with 5% fetal bovine serum (FBS). Cells were then stained with streptavidin-coupled APC (Biolegend #405207) for 5 minutes at 4° C. and washed and resuspended in PBS with 5% FBS. Antigen binding B cells were then sorted using a Sony MA900 cell sorter by gating on live cells in the forward scatter and side scatter (FIGS. 1A and 1B) and on CD19-FITC and SARS-COV-2 S-APC or RBD-APC double-positive cells. After sorting, cells were washed and counted using a hemocytometer and microscopy, before resuspending up to 10,000 cells in a volume of 32 μl for 5′ single cell RNA-Seq (see below).

5′ scRNA-seq library generation. Cells were separated into droplet emulsions using the Chromium Next GEM Single-cell 5′ Solution (v1.1) and the 10× Chromium Controller. 5,000-10,000 cells were loaded per channel of the Chromium Next GEM single-cell 5′ (v1.1) Chip G. Following lysis of cells, barcoded mRNA reverse transcription, and cDNA amplification, a 0.6× SPRI cleanup was performed and the supernatant was set aside for Feature Barcoding library construction as instructed by the Chromium NextGEM single-cell V(D)J v1.1 protocol. A final elution of 45 μL was saved for further construction of libraries. Using the saved supernatant of the 0.6× cDNA cleanup, Feature Barcoding libraries were completed according to the 5′ Next GEM (v1.1) Feature Barcoding library construction methods provided by 10× Genomics. Gene expression and V(D)J libraries were created according to manufacturer's instruction (10× Genomics), which includes enzymatic fragmentation, adaptor ligation, and sample index barcoding steps. The V(D)J libraries were created from the original cDNA after it was enriched for human B cells, following the Chromium NextGEM single-cell V(D)J v1.1 protocol.

sc RNA-seq library sequencing. Gene expression, feature barcoding libraries, and BCR enriched V(D)J libraries were sequenced on a Nextseq500 (Illumina) using a high output 150 cycle flowcell, with the read configuration Read 1: 28 cycles, Read 2: 96 cycles, Index read 1: 8 cycles or sequenced on a HiSeq X (Illumina), using a 150 cycle flowcell with the read configuration: Read 1: 28 cycles, Read 2: 96 cycles, Index read 1: 8 cycles. Feature Barcoding libraries were spiked into the gene expression libraries (at 10-20% of the sample pool) prior to sequencing. All BCR enriched V(D)J libraries were pooled together and sequenced on a NextSeq500 (Illumina) using the same parameters as previously mentioned.

Antibody production. Antibody VDJ heavy chain and VJ light chain sequences of selected antibodies were produced as minigenes and cloned into IgG1 heavy chain, IgA1 heavy chain, IgA2 heavy chain, kappa light chain or lambda light chain expression vectors as previously described (Wardemann et al., 2003) (Wang et al., 2020b). After cloning into expression vectors, matching antibody heavy chain and light chain plasmids were co-transfected into Expi293F cells following the manufacturer's instructions. In brief, heavy chain plasmid DNA and light chain plasmid DNA were diluted in 1.5 ml Opti-Plex Complexation Buffer (Invitrogen) before mixing with 80 μl ExpiFectamine 293 Reagent (Invitrogen) diluted in 1.4 ml Opti-Plex Complexation Buffer (Invitrogen). For dimeric IgA production equal amounts of heavy chain plasmid DNA, light chain plasmid DNA and J chain plasmid DNA were used in the transfection. Mixture was incubated for 15 minutes at room temperature before adding to 25 ml of Expi293F cells at a density of 3.0×10⁶ viable cells/ml and incubation in a shaker incubator according to the manufacturer's instructions. ExpiFectamine 293 Transfection Enhancer 1 and 2 (150 μl and 1.5 ml, respectively) were added 18 hours post-transfection. Antibody containing supernatants were harvested after 7 days by centrifugation of cells at 3,000g for 20 minutes and transfer of supernatants into 50 ml falcon tubes (Fisher Scientific). Prior to size exclusion chromatography (see below) IgA dimers were purified from transfection supernatants using peptide M coupled agarose beads (Invivogen) according to the manufacturer's instructions.

IgA dimer purification through size exclusion chromatography. A pre-packed HiLoad™ 16/600 Superdex™ 200 pg (Cytiva, #28-9893-35) column on the Cytiva “ÄKTA pure Chromatography System” was equilibrated with PBS. After equilibration of the column with PBS each IgA preparation was applied onto the column and the column was run at a flow rate of 1.25 ml/min. The total column volume was 120 ml. Fractions were collected in a volume of 1.5 ml per fraction. Peaks consistent with IgA multimers, IgA dimers and IgA monomers were detected at 0.3-0.4, 0.4-0.5 and 0.5-0.6 column volumes respectively (FIG. 9J). Fractions spanning all three peaks were collected and evaluated individually by running Criterion™ TGX™ precast gels (Biorad 5671095) under non-reducing conditions with a Precision Plus Protein™ Kaleidoscope™ Prestained Protein Standard (Biorad #1610375) (FIG. 9K).

Antibody ELISA testing. Antibody concentrations were determined measuring absorbance at 280 nm using Nanodrop 2000c (Thermo Scientific) or IgG specific ELISA as previously described (Tiller et al., 2008). Antibody reactivities to SARS-COV-2 S, SARS-COV-2 RBD, SARS-COV S, SARS-COV RBD, MERS-COV S and MERS-COV RBD were determined using the same protocol with the following modifications: Antigens were coated on Corning™ Costar™ Brand 96-Well EIA/RIA Plates at a concentration of 5 μg/ml. Antibody binding was then assessed at starting concentrations of 1 μg/ml, 1.1 μg/ml and 1.2 μg/ml for IgG, monomeric IgA and dimeric IgA respectively in order to achieve equal Fab molar concentrations. 3 consecutive 1/4 dilutions were performed. Positive control antibodies used in these assays included antibodies BG10-19 for SARS-COV-2 S and SARS-COV-2 RBD (see above), 3B12 (Absolute Antibody #Ab01673-10.0) for MERS-COV S and MERS-COV RBD (Tang et al., 2014) and S227.14 (Absolute Antibody Ab00263-10.0) for SARS COV S and SARS COV RBD (Rockx et al., 2008). Antigen specific ELISA results are expressed as AUC using Graphpad PRISM software. Polyreactivity ELISAs were performed as previously described (Tiller et al., 2008) with the following modification: In addition to the antigens ssDNA, dsDNA, LPS and insulin (Tiller et al., 2008), streptavidin-coupled APC (Biolegend #405207) was used as a fifth antigen in order to assess potential off target binding against this reagent used for cell sorting (see above). As described previously (Tiller et al., 2008), polyreactivity is defined as reactivity to 2 or more antigens among the antigens single stranded DNA, double stranded DNA, lipopolysaccharide (LPS) or insulin. As previously described, antibodies ED38, JB40 and mGO53 (Wardemann et al., 2003) were used as strongly polyreactive, intermediately polyreactive and non-polyreactive control antibodies respectively. In ELISA assays including IgG, IgA monomers and IgA dimers, HRP-conjugated goat anti-human kappa and lambda chain antibodies (Biorad #STAR127P and #STAR129P) were used as secondary antibodies at a 1/5000 dilution.

Off-target antibody binding to baculovirus (BV) particles generated in Sf9 insect cells was tested as previously described (Hotzel et al., 2012). A solution of 1% baculovirus in 100 mM sodium bicarbonate buffer pH 9.6 was adsorbed to a 384-well ELISA plate (Nunc Maxisorp) using a Tecan Freedom Evo2 liquid handling robot and the plate was incubated overnight at 4° C. Following blocking with 0.5% BSA in PBS, 1 μg/ml of IgG was added to the blocked assay. Plates were incubated for 3 hours at room temperature. Antibody binding was detected using an HRP-conjugated anti-Human IgG (H&L) secondary antibody (Genscript). ELISA was developed using SuperSignal ELISA Femto Maximum Sensitivity Substrate (Thermo Scientific). Anti-HIV antibodies NIH45-46 (Scheid et al., 2011) and 45-46m2 (Diskin et al., 2013) were used as positive controls and antibody 3BNC117 (Scheid et al., 2011) was used as negative control. Measurements were performed in quadruplicate and OD values within 1.5-fold the negative control were considered to be negative.

Neutralization activity of antibodies against authentic SARS-COV-2. Vero E6-TMPRSS2 were seeded at 10,000 per well the day prior to infection in CellCarrier-384 ultra microplate (Perkin Elmer). Monoclonal antibody samples were tested in 4-fold 9-point dilution spots starting at a highest concentration of 100 μg/mL. Serial diluted antibodies were mixed separately with diluted SARS-COV-2 virus and incubated at 37 C with 5% CO₂ for 1 hour. Antibody-virus complexes were added to the cells in triplicate. Plates were incubated at 37 C with 5% CO₂ for 48 hours. After that, plates were fixed and inactivated using 4% paraformaldehyde in PBS for 2 hours at room temperature. Plates were then washed and incubated with diluted anti-SARS-COV/SARS-COV-2 nucleoprotein mouse antibody (Sino) for 1.5 hours at room temperature. Plates were subsequently incubated with Alexa488-conjugated goat anti-mouse (JacksonImmuno) for 45 mins at room temperature, followed by nuclear staining with Hoechst 33342 (ThermoFisher). The fluorescence images were recorded and analyzed using Opera Phenix™ High Content Screening System. The half-maximal inhibitory concentrations (I₅₀) were determined using four parameters logistic regression (GraphPad Prism 8.0).

Protein expression and purification. Expression and purification of SARS-COV-2 6P stabilized S trimers (Hsieh et al., 2020) and constructs encoding the sarbecovirus RBDs were conducted as previously described (Cohen et al., 2020). Briefly, constructs were purified from supernatants of transiently transfected Expi293F cells (Gibco) by Ni2⁺-NTA affinity and size exclusion chromatography (SEC). Peak fractions were identified by SDS-PAGE, pooled, and stored at 4° C. IgGs were expressed, purified, and stored as described (Barnes et al., 2020b). Fabs were generated by papain digestion using crystallized papain (Sigma-Aldrich) in 50 mM sodium phosphate, 2 mM EDTA, 10 mM L-cysteine, pH 7.4 for 30-60 min at 37° C. at a 1:100 enzyme:IgG ratio. To remove undigested IgGs and Fc fragments, digested products were applied to a 1-mL HiTrap MabSelect SuRe column (GE Healthcare Life Sciences) and the flow-through containing cleaved Fabs was collected. Fabs were further purified by SEC using a Superdex 200 Increase 10/300 column (GE Healthcare Life Sciences) in TBS before concentrating and storage at 4° C.

Cryo-EM sample preparation. Purified Fab and S 6P trimer were incubated at a 1.1:1 molar ratio per protomer on ice for 30 minutes prior to deposition on a freshly glow-discharged 300 mesh, 1.2/1.3 UltrAuFoil grid. Immediately before 3 μl of complex was applied to the grid, fluorinated octyl-malotiside was added to the Fab-S complex to a final detergent concentration of 0.02% w/v, resulting in a final complex concentration of 3 mg/ml. Samples were vitrified in 100% liquid ethane using a Mark IV Vitrobot after blotting for 3 s with Whatman No. 1 filter paper at 22° C. and 100% humidity.

Cryo-EM data collection and processing. Data collection and processing followed a similar workflow to what has been previously described in detail (Barnes et al., 2020a). Briefly, micrographs were collected on a Talos Arctica transmission electron microscope (Thermo Fisher) operating at 200 kV for all Fab-S complexes. Data were collected using SerialEM automated data collection software (Mastronarde, 2005) and movies were recorded with K3 camera (Gatan). Data collections parameters are summarized in Table 5. For all data sets, cryo-EM movies were patch motion corrected for beam-induced motion including dose-weighting within cryoSPARC v2.15 (Punjani et al., 2017) after binning super resolution movies. The non-dose-weighted images were used to estimate CTF parameters using cryoSPARC implementation of the Patch CTF job. Processing for all datasets was carried out in a similar fashion. Briefly, an initial set of particles was picked based on templates from 2D classification of blob picked particles on a small sub-set of images. This set was pared down through several rounds of 3D classification. An ab initio job on a small good subset of these particles revealed distinct states and junk particles. Full set of particles was heterogeneously refined against distinct states, as well as a junk class acting as a trap for bad particles. Particles from each were separately refined using non-uniform refinement in C1 symmetry. Particles from distinct states were re-extracted without binning and were further refined separately in several rounds of 3D classification. Particles were subdivided into groups based on beam-tilt, refined separately for CTF parameters and aberration correction. For all states, a soft mask (3-pixel extension, 6-pixel soft edge) was generated for the S1 subunits and Fab variable domains. The resolution at the Fab interface was modestly improved, with overall reported resolutions reported based on gold standard FSC calculations.

Cryo-EM Structure Modeling and Refinement. Coordinates for initial complexes were generated by docking individual chains from reference structures into cryo-EM density using UCSF Chimera (Goddard et al., 2018) (see Table 5 for PDB coordinates). Models were then refined into cryo-EM maps rigid body and real space refinement with morphing in Phenix (Terwilliger et al., 2018). Sequence-updated models were built manually in Coot (Emsley et al., 2010) and then refined using iterative rounds of real-space refinement in Phenix and Coot. Glycans were modeled at potential N-linked glycosylation sites (PNGSs) in Coot using ‘blurred’ maps processed with a variety of B-factors generated in cryoSPARC v2.15. Validation of model coordinates was performed using MolProbity (Chen et al., 2010) (Table 5).

BG4-25-RBD X-ray crystallography experiments. Crystallization trials for a stoichiometric complex of BG4-25-SARS-COV2 RBD-CR3022 were carried out at room temperature using the sitting drop vapor diffusion method by mixing equal volumes of the Fab-RBD complex and reservoir using a TTP LabTech Mosquito robot and commercially-available screens (Hampton Research). Crystals were obtained in 0.05 M Citric acid, 0.05 M BIS-TRIS propane pH 5.0 and 16% polyethylene glycol 3350 and quickly cryo-protected in a solution matching the reservoir+20% glycerol. X-ray diffraction data were collected for Fab-RBD complex at the Stanford Synchrotron Radiation Lightsource (SSRL) beamline 12-2 on a Pilatus 6M pixel detector (Dectris). Data from a single crystal were indexed and integrated in XDS (Kabsch, 2010) and merged using AIMLESS in CCP4 (Winn et al., 2011) (Table 6). The Fab-RBD complex structure was determined by molecular replacement in PHASER (McCoy et al., 2007) using Fab and RBD coordinates from individual components of PDB 6XC3 (CC12.1 Fab-CR3022 Fab-SARS-COV2 RBD) as search models after trimming heavy chain and light chain CDR loops for CC12.1 Fab. Coordinates were refined using rigid body and B-group refinement in Phenix (Adams et al., 2010) followed by cycles of manual building in Coot (Emsley et al., 2010) (Table 6).

Structural Analyses. CDR lengths and Kabot numbering were calculated based on IMGT definitions (Lefranc et al., 2015). Structure figures were made with UCSF ChimeraX. Local resolution maps were calculated using cryoSPARC v 2.15. Buried surface areas were calculated using PDBePISA (Krissinel and Henrick, 2007) and a 1.4 Å probe. Potential hydrogen bonds were assigned as interactions that were <4.0 Å and with A-D-H angle >90°. Potential van der Waals interactions between atoms were assigned as interactions that were <4.0 Å. Hydrogen bond and van der Waals interaction assignments are tentative due to resolution limitations.

Surface Plasmon Resonance (SPR) binding experiments. SPR experiments were performed using a Biacore T200 instrument (GE Healthcare). ACE2 microbody (Tada et al., 2020) was immobilized on a CM5 chip by primary amine chemistry at pH 4.5 to a final response level of ˜1000 resonance units (RUS). Fabs were complexed with 100 nM SARS2 S 6P or SARS2 RBD at a 10:1 molar ratio and incubated for a minimum of 1 hour. Antigen or Fab-antigen complex was injected over immobilized ACE2 microbody surface at a flow rate of 30 μL/min for a contact time of 300 seconds. Sensorgrams were buffer corrected using an injection of 0.01 M HEPES pH 7.4, 0.15 M NaCl, 3 mM EDTA, 0.005% v/v surfactant P20 buffer.

SARS-COV-2 mutant RBD and sarbecovirus RBD ELISA binding assay. Binding of Fabs to SARS-COV-2 RBDs containing single mutations or to sarbecovirus RBDs was evaluated by ELISA. RBD antigens (mutant or wild-type) were adsorbed to 384-well Nunc MaxiSorp plates (Sigma) at a concentration of 2 μg/mL overnight. Plates were blocked with 3% BSA in TBS-T (TBS with 0.05% Tween20) for 1 h at room temperature, then 5-fold serial dilutions starting at 10 μg/mL of Fab were added. Plates were washed with TBS-T and bound Fab was detected using an HRP-conjugated secondary Ab (Genscript) and SuperSignal ELISA Femto Substrate (Thermo Scientific). AUC for each Fab-antigen pair was calculated using Graphpad PRISM software. Fold decrease in AUC was calculated relative to SARS-COV2 RBD AUC for the same Fab. Data shown are representative of two independent experiments.

SARS-COV-2 mutant pseudotyped reporter virus and mutant pseudotyped virus neutralization assay. SARS-COV-2 pseudotyped particles were generated as previously described (Robbiani et al., 2020; Schmidt et al., 2020). Briefly, 293T cells were transfected with pNL4-3 Env-nanoluc and pSARS-COV-2-S_{Δ19}. For generation of RBD-mutant pseudoviruses, pSARS-CoV-2-S_{Δ19} carrying indicated spike mutations was used instead. Particles were harvested 48 hpt, filtered and stored at −80° C.

Fourfold serially diluted monoclonal antibodies were incubated with SARS-COV-2 pseudotyped virus for 1 h at 37° C. The mixture was subsequently incubated with HT1080_{Ace2} cl14 cells (Schmidt et al., 2020) for 48 h after which cells were washed with PBS and lysed with Luciferase Cell Culture Lysis 5× reagent (Promega). Nanoluc Luciferase activity in lysates was measured using the Nano-Glo Luciferase Assay System (Promega) with the Glomax Navigator (Promega). The obtained relative luminescence units were normalized to those derived from cells infected with SARS-COV-2 pseudotyped virus in the absence of monoclonal antibodies. The half-maximal and 90% inhibitory concentrations (IC50 and IC90) were determined using four-parameter nonlinear regression (least squares regression method without weighting; constraints: top=1, bottom=0) (GraphPad Prism).

Antibody resistance selection experiments. 293T/ACE2.cl22 (Schmidt et al., 2020) were cultured in Dulbecco's Modified Eagle Medium (DMEM) supplemented with 10% fetal bovine serum (FBS) at 37° C. and 5% CO₂. Cells have been tested negative for contamination with mycoplasma. rVSV/SARS-COV-2/GFP chimeric virus stocks were generated by infecting 293T/ACE2.cl22 cells. Supernatant was harvested 1 day post infection (dpi), cleared from cellular debris, aliquoted and stored at −80° C. Two plaque purified variants designated rVSV/SARS-COV-2/GFP1D7 and rVSV/SARS-COV-2/GFP2E1 that encode F157S/R685M (1D7) and D215G/R683G (2E1) substitutions were used in these studies (Schmidt et al., 2020). For BG10-19 selection experiments, the virus was passaged multiple times in the presence of 1 μg/ml or 5 μg/ml antibody. At passage 4, virus was allowed to replicate in the presence of antibodies until all the cells were infected then harvested. The virus was then passaged in the presence of antibodies for a fifth and final time before supernatant was harvested for further analysis. To isolate individual mutants, the supernatants of passage 5 were serially diluted and individual viral foci were isolated by limiting dilution in 96-well plates.

For the identification of putative antibody resistance mutations, RNA was isolated from aliquots of supernatant containing selected viral populations and isolates using NucleoSpin 96 Virus Core Kit (Macherey-Nagel). The purified RNA was subjected to reverse transcription using SuperScript VILO cDNA Synthesis Kit (Thermo Fisher Scientific). The cDNA was amplified using KOD Xtreme Hot Start DNA Polymerase (Millipore Sigma) and primers flanking the entire S-encoding sequence. The PCR products were purified and sequenced using Sanger-sequencing.

To measure neutralizing activity, 40 μg/ml antibody was five-fold serially diluted in 96-well plates over 7 dilutions. Thereafter, approximately 5×10⁴ infectious units of rVSV/SARS-CoV-2/GFP WT or mutant isolates were mixed with BG10-19 at a 1:1 ratio and incubated for 1 hr at 37° C. in a 96-well plate. The mixture was then added to 293T/ACE2.cl22 target cells plated at 1×10⁴ cells/well in 100 μl medium in 96-well plates the previous day. Thus, the final starting dilution was 10 μg/ml. Cells were then cultured for 16 hr, then harvested for flow cytometry.

scRNA-seq analysis. mRNA and VDJ sequence reads were mapped to the reference human genome GRCh38-3.0.0 with the cloud-based Cumulus workflows (Li et al., 2020), using the CellRanger 3.0.2 software pipeline. Cells with both high-quality VDJ sequence and transcriptome information were kept for the downstream analysis by filtering out the cells which had less than 300 detected genes or which had poor quality VDJ contig information defined by i) being non-productive by 10× standards, ii) having more than four productive VDJ contigs, iii) having less than three filtered UMIs. For the transcriptome analysis, batch effects were removed with the ComBat algorithm (Johnson et al., 2007) implemented in SVA R Package version 3.38.0. For the transcriptome mRNA count normalization, dimensionality reduction, clustering, cell cycle scoring, cluster marker genes detection and differential gene expression analysis steps Seurat R package (Butler et al., 2018) (Stuart et al., 2019) was employed. For the normalization step gene expression counts for each cell were divided by the total counts for that cell and multiplied by 1e6, which was then log-transformed using log1p. Dimensionality reduction was done by PCA with selecting 50 first principal components.

For clustering of the cells into transcriptome clusters, first the k-nearest neighbor (kNN) graph of the cells was constructed. Second, this kNN graph was used to generate the shared nearest neighbor (sNN) graph by calculating Jaccard index between every cell and its k nearest neighbors. Third, the leiden algorithm (Traag et al., 2019) was used to find the clusters of the cells based on the generated sNN graph.

Cell cycle scoring was done by calculating the module scores of the cell cycle genes defined in (Tirosh et al., 2016). Positive cluster marker genes and differentially expressed genes were detected with a log-fold change threshold of 0.25, where only the genes that were detected in a minimum fraction of 20% in either of the six transcriptome populations were considered. Expression levels of immunoglobulin genes were discarded during the clustering and differential gene expression analysis steps.

Antibody Repertoire Analysis. 10×V(D)J contig assembly algorithm takes many forms of noise specific to scRNA-seq data into account while generating the assembled V(D)J sequences (support. 10×genomics.com/single-cell-vdj/software/pipelines/latest/algorithms/assembly). Nevertheless, only the cells with high-quality V(D)J contig sequences were selected and V(D)J gene annotations were assigned by using IGBLAST (version 1.14.0) software with the Change-O R package (Gupta et al., 2015). Donor specific B cell clones were identified by the Change-O R package (Gupta et al., 2015), where the appropriate threshold for trimming the hierarchical clustering into B cell clones was found by inspecting the bimodal distribution of the distance between each sequence in the data and its nearest-neighbor.

Mutation inference based on the scRNA-seq VDJ sequences of the donor and the control cells (Rubelt et al., 2012) was performed by the Shazam R Package (Yaari et al., 2012) where the region definition parameter was set to be “IMGT_V_BY_SEGMENTS” which provides no subdivisons and treats the entire V segment as a single region.

CDRH3 length was defined based on IMGT definition (Lefranc et al., 2015) with the addition of two conserved amino acid residues that were added to assist in clonal analysis (Nouri and Kleinstein, 2018). This addition was corrected for all analyses involving specific CDRH3 length, such as selection of VH3-53/3-66 antibodies with CDRH3 shorter than 14 amino acid length.

CDRH3 amino acid charges were calculated by the Alakazam R package (Gupta et al., 2015) using the method of Moore (Moore, 1985) excluding the N-terminus and C-terminus charges, and normalizing by the number of informative positions.

Hydrophobicity scores were calculated with the Alakazam R package using the method of Kyte and Doolittle (Kyte and Doolittle, 1982).

Shannon entropy values were calculated using Alakazam R package. For each donor the transcriptome cluster specific Hill diversity index, proposed in (Hill, 1973), improved by (Chao et al., 2014; Chao et al., 2015) was calculated by setting the diversity order equal to 1 with Alakazam R package. For each run the number of bootstrap realizations is set to be 400, and the minimum number of observations to sample is set to be 10.

Mutational analysis of VH3-53/3-66 antibodies. Inferred somatic mutations in the V gene segment of VH3-53/VH3-66 antibodies were counted for the IgG⁺ antibodies with CDRH3 lengths less than 14 amino acids from SARS-COV-2 binding B cells across 14 subjects, listing only those sites where the frequency was >10% in either the IgG⁺ set or in the repertoire comparison. The repertoire comparison set of human VH3-53/VH3-66 sequences was taken from the sequence read archive of (Rubelt et al., 2012).

Table 8A-8B. The Table lists the nucleotide sequence (SEQ ID NOS: 447-12672), the bait that was used to sort the respective cell and the sequence name. The sequence name includes the donor (e.g., BG1, BG10) and whether the sequence is a light chain or heavy chain (e.g., LC, HC). The nucleotide sequences can be converted to amino acid sequence by one skilled in the art. 8A. Full list of nucleotide sequences for heavy and light chains identified in B cells bound to bait proteins from the 14 donors. 8B. For patients BG1, BG4, BG7 and BG10 there is an additional column that shows the antibody names for the antibodies produced from the nucleotide sequences in that line.

TABLE 8A

| SEQ | | |
|---|---|---|
| ID NO | BAIT | SEQUENCE NAME |
| 447 | RBD | BG1NUC-HC1 |
| 448 | 2F | BG1NUC-HC2 |
| 449 | RBD | BG1NUC-HC3 |
| 450 | 2F | BG1NUC-HC4 |
| 451 | RBD | BG1NUC-HC5 |
| 452 | 2F | BG1NUC-HC6 |
| 453 | RBD | BG1NUC-HC7 |
| 454 | RBD | BG1NUC-HC8 |
| 455 | RBD | BG1NUC-HC9 |
| 456 | RBD | BG1NUC-HC10 |
| 457 | RBD | BG1NUC-HC11 |
| 458 | 2F | BG1NUC-HC12 |
| 459 | RBD | BG1NUC-HC13 |
| 460 | RBD | BG1NUC-HC14 |
| 461 | RBD | BG1NUC-HC15 |
| 462 | RBD | BG1NUC-HC16 |
| 463 | RBD | BG1NUC-HC17 |
| 464 | 2P | BG1NUC-HC18 |
| 465 | 2P | BG1NUC-HC19 |
| 466 | RBD | BG1NUC-HC20 |
| 467 | 2P | BG1NUC-HC21 |
| 468 | 2P | BG1NUC-HC22 |
| 469 | 2P | BG1NUC-HC23 |
| 470 | 2P | BG1NUC-HC24 |
| 471 | RBD | BG1NUC-HC25 |
| 472 | 2P | BG1NUC-HC26 |
| 473 | RBD | BG1NUC-HC27 |
| 474 | 2P | BG1NUC-HC28 |
| 475 | RBD | BG1NUC-HC29 |
| 476 | 2P | BG1NUC-HC30 |
| 477 | RBD | BG1NUC-HC31 |
| 478 | RBD | BG1NUC-HC32 |
| 479 | 2P | BG1NUC-HC33 |
| 480 | 2P | BG1NUC-HC34 |
| 481 | RBD | BG1NUC-HC35 |
| 482 | 2P | BG1NUC-HC36 |
| 483 | RBD | BG1NUC-HC37 |
| 484 | RBD | BG1NUC-HC38 |
| 485 | 2P | BG1NUC-HC39 |
| 486 | RBD | BG1NUC-HC40 |
| 487 | RBD | BG1NUC-HC41 |
| 488 | 2P | BG1NUC-HC42 |
| 489 | 2P | BG1NUC-HC43 |
| 490 | RBD | BG1NUC-HC44 |
| 491 | RBD | BG1NUC-HC45 |
| 492 | RBD | BG1NUC-HC46 |
| 493 | 2P | BG1NUC-HC47 |
| 494 | RBD | BG1NUC-HC48 |
| 495 | RBD | BG1NUC-HC49 |
| 496 | RBD | BG1NUC-HC50 |
| 497 | 2P | BG1NUC-HC51 |
| 498 | 2P | BG1NUC-HC52 |
| 499 | RBD | BG1NUC-HC53 |
| 500 | 2P | BG1NUC-HC54 |
| 501 | 2P | BG1NUC-HC55 |
| 502 | RBD | BG1NUC-HC56 |
| 503 | 2P | BG1NUC-HC57 |
| 504 | 2P | BG1NUC-HC58 |
| 505 | 2P | BG1NUC-HC59 |
| 506 | RBD | BG1NUC-HC60 |
| 507 | RBD | BG1NUC-HC61 |
| 508 | RBD | BG1NUC-HC62 |
| 509 | 2P | BG1NUC-HC63 |
| 510 | 2P | BG1NUC-HC64 |
| 511 | 2P | BG1NUC-HC65 |
| 512 | 2P | BG1NUC-HC66 |
| 513 | RBD | BG1NUC-HC67 |
| 514 | RBD | BG1NUC-HC68 |
| 515 | 2P | BG1NUC-HC69 |
| 516 | 2P | BG1NUC-HC70 |
| 517 | 2P | BG1NUC-HC71 |
| 518 | RBD | BG1NUC-HC72 |
| 519 | RBD | BG1NUC-HC73 |
| 520 | RBD | BG1NUC-HC74 |
| 521 | 2P | BG1NUC-HC75 |
| 522 | RBD | BG1NUC-HC76 |
| 523 | 2P | BG1NUC-HC77 |
| 524 | 2P | BG1NUC-HC78 |
| 525 | 2P | BG1NUC-HC79 |
| 526 | RBD | BG1NUC-HC80 |
| 527 | RBD | BG1NUC-HC81 |
| 528 | 2P | BG1NUC-HC82 |
| 529 | RBD | BG1NUC-HC83 |
| 530 | 2P | BG1NUC-HC84 |
| 531 | 2P | BG1NUC-HC85 |
| 532 | 2P | BG1NUC-HC86 |
| 533 | RBD | BG1NUC-HC87 |
| 534 | 2P | BG1NUC-HC88 |
| 535 | RBD | BG1NUC-HC89 |
| 536 | RBD | BG1NUC-HC90 |
| 537 | RBD | BG1NUC-HC91 |
| 538 | RBD | BG1NUC-HC92 |
| 539 | RBD | BG1NUC-HC93 |
| 540 | RBD | BG1NUC-HC94 |
| 541 | RBD | BG1NUC-HC95 |
| 542 | RBD | BG1NUC-HC96 |
| 543 | 2P | BG1NUC-HC97 |
| 544 | 2P | BG1NUC-HC98 |
| 545 | 2P | BG1NUC-HC99 |
| 546 | RBD | BG1NUC-HC100 |
| 547 | 2P | BG1NUC-HC101 |
| 548 | RBD | BG1NUC-HC102 |
| 549 | 2P | BG1NUC-HC103 |
| 550 | 2P | BG1NUC-HC104 |
| 551 | 2F | BG1NUC-HC105 |
| 552 | 2P | BG1NUC-HC106 |
| 553 | RBD | BG1NUC-HC107 |
| 554 | RBD | BG1NUC-HC108 |
| 555 | 2P | BG1NUC-HC109 |
| 556 | 2P | BG1NUC-HC110 |
| 557 | 2P | BG1NUC-HC111 |
| 558 | 2P | BG1NUC-HC112 |
| 559 | 2P | BG1NUC-HC113 |
| 560 | RBD | BG1NUC-HC114 |
| 561 | RBD | BG1NUC-HC115 |
| 562 | RBD | BG1NUC-HC116 |
| 563 | RBD | BG1NUC-HC117 |
| 564 | 2P | BG1NUC-HC118 |
| 565 | 2P | BG1NUC-HC119 |
| 566 | RBD | BG1NUC-HC120 |
| 567 | 2P | BG1NUC-HC121 |
| 568 | 2P | BG1NUC-HC122 |
| 569 | 2P | BG1NUC-HC123 |
| 570 | RBD | BG1NUC-HC124 |
| 571 | RBD | BG1NUC-HC125 |
| 572 | RBD | BG1NUC-HC126 |
| 573 | RBD | BG1NUC-HC127 |
| 574 | RBD | BG1NUC-HC128 |
| 575 | RBD | BG1NUC-HC129 |
| 576 | RBD | BG1NUC-HC130 |
| 577 | RBD | BG1NUC-HC131 |
| 578 | RBD | BG1NUC-HC132 |
| 579 | RBD | BG1NUC-HC133 |
| 580 | 2P | BG1NUC-HC134 |
| 581 | 2P | BG1NUC-HC135 |
| 582 | RBD | BG1NUC-HC136 |
| 583 | RBD | BG1NUC-HC137 |
| 584 | RBD | BG1NUC-HC138 |
| 585 | RBD | BG1NUC-HC139 |
| 586 | 2P | BG1NUC-HC140 |
| 587 | 2P | BG1NUC-HC141 |
| 588 | 2P | BG1NUC-HC142 |
| 589 | 2P | BG1NUC-HC143 |
| 590 | 2P | BG1NUC-HC144 |
| 591 | 2P | BG1NUC-HC145 |
| 592 | 2P | BG1NUC-HC146 |
| 593 | 2P | BG1NUC-HC147 |
| 594 | 2P | BG1NUC-HC148 |
| 595 | 2P | BG1NUC-HC149 |
| 596 | RBD | BG1NUC-HC150 |
| 597 | 2P | BG1NUC-HC151 |
| 598 | RBD | BG1NUC-HC152 |
| 599 | RBD | BG1NUC-HC153 |
| 600 | 2P | BG1NUC-HC154 |
| 601 | 2P | BG1NUC-HC155 |
| 602 | RBD | BG1NUC-HC156 |
| 603 | 2P | BG1NUC-HC157 |
| 604 | 2P | BG1NUC-HC158 |
| 605 | 2F | BG1NUC-HC159 |
| 606 | 2P | BG1NUC-HC160 |
| 607 | RBD | BG1NUC-HC161 |
| 608 | 2P | BG1NUC-HC162 |
| 609 | RBD | BG1NUC-HC163 |
| 610 | RBD | BG1NUC-HC164 |
| 611 | RBD | BG1NUC-HC165 |
| 612 | RBD | BG1NUC-HC166 |
| 613 | 2P | BG1NUC-HC167 |
| 614 | RBD | BG1NUC-HC168 |
| 615 | RBD | BG1NUC-HC169 |
| 616 | 2P | BG1NUC-HC170 |
| 617 | 2P | BG1NUC-HC171 |
| 618 | 2P | BG1NUC-HC172 |
| 619 | RBD | BG1NUC-HC173 |
| 620 | RBD | BG1NUC-HC174 |
| 621 | RBD | BG1NUC-HC175 |
| 622 | 2P | BG1NUC-HC176 |
| 623 | RBD | BG1NUC-HC177 |
| 624 | RBD | BG1NUC-HC178 |
| 625 | 2P | BG1NUC-HC179 |
| 626 | RBD | BG1NUC-HC180 |
| 627 | RBD | BG1NUC-HC181 |
| 628 | RBD | BG1NUC-HC182 |
| 629 | RBD | BG1NUC-HC183 |
| 630 | 2P | BG1NUC-HC184 |
| 631 | RBD | BG1NUC-HC185 |
| 632 | RBD | BG1NUC-HC186 |
| 633 | RBD | BG1NUC-HC187 |
| 634 | RBD | BG1NUC-HC188 |
| 635 | 2P | BG1NUC-HC189 |
| 636 | RBD | BG1NUC-HC190 |
| 637 | 2P | BG1NUC-HC191 |
| 638 | 2P | BG1NUC-HC192 |
| 639 | 2P | BG1NUC-HC193 |
| 640 | 2P | BG1NUC-HC194 |
| 641 | 2P | BG1NUC-HC195 |
| 642 | 2P | BG1NUC-HC196 |
| 643 | 2P | BG1NUC-HC197 |
| 644 | 2P | BG1NUC-HC198 |
| 645 | 2P | BG1NUC-HC199 |
| 646 | RBD | BG1NUC-HC200 |
| 647 | RBD | BG1NUC-HC201 |
| 648 | RBD | BG1NUC-HC202 |
| 649 | 2P | BG1NUC-HC203 |
| 650 | 2P | BG1NUC-HC204 |
| 651 | 2P | BG1NUC-HC205 |
| 652 | RBD | BG1NUC-HC206 |
| 653 | 2P | BG1NUC-HC207 |
| 654 | 2P | BG1NUC-HC208 |
| 655 | 2P | BG1NUC-HC209 |
| 656 | RBD | BG1NUC-HC210 |
| 657 | RBD | BG1NUC-HC211 |
| 658 | 2P | BG1NUC-HC212 |
| 659 | 2P | BG1NUC-HC213 |
| 660 | 2P | BG1NUC-HC214 |
| 661 | 2P | BG1NUC-HC215 |
| 662 | RBD | BG1NUC-HC216 |
| 663 | 2P | BG1NUC-HC217 |
| 664 | 2P | BG1NUC-HC218 |
| 665 | RBD | BG1NUC-HC219 |
| 666 | 2P | BG1NUC-HC220 |
| 667 | 2P | BG1NUC-HC221 |
| 668 | 2P | BG1NUC-HC222 |
| 669 | 2P | BG1NUC-HC223 |
| 670 | RBD | BG1NUC-HC224 |
| 671 | RBD | BG1NUC-HC225 |
| 672 | 2P | BG1NUC-HC226 |
| 673 | 2P | BG1NUC-HC227 |
| 674 | 2P | BG1NUC-HC228 |
| 675 | RBD | BG1NUC-HC229 |
| 676 | RBD | BG1NUC-HC230 |
| 677 | RBD | BG1NUC-HC231 |
| 678 | 2P | BG1NUC-HC232 |
| 679 | RBD | BG1NUC-HC233 |
| 680 | RBD | BG1NUC-HC234 |
| 681 | 2P | BG1NUC-HC235 |
| 682 | 2P | BG1NUC-HC236 |
| 683 | 2P | BG1NUC-HC237 |
| 684 | 2P | BG1NUC-HC238 |
| 685 | RBD | BG1NUC-HC239 |
| 686 | RBD | BG1NUC-HC240 |
| 687 | 2P | BG1NUC-HC241 |
| 688 | 2P | BG1NUC-HC242 |
| 689 | 2P | BG1NUC-HC243 |
| 690 | RBD | BG1NUC-HC244 |
| 691 | RBD | BG1NUC-HC245 |
| 692 | RBD | BG1NUC-HC246 |
| 693 | 2P | BG1NUC-HC247 |
| 694 | 2P | BG1NUC-HC248 |
| 695 | 2P | BG1NUC-HC249 |
| 696 | RBD | BG1NUC-HC250 |
| 697 | RBD | BG1NUC-HC251 |
| 698 | 2P | BG1NUC-HC252 |
| 699 | RBD | BG1NUC-HC253 |
| 700 | RBD | BG1NUC-HC254 |
| 701 | RBD | BG1NUC-HC255 |
| 702 | 2P | BG1NUC-HC256 |
| 703 | 2P | BG1NUC-HC257 |
| 704 | 2P | BG1NUC-HC258 |
| 705 | 2P | BG1NUC-HC259 |
| 706 | RBD | BG1NUC-HC260 |
| 707 | RBD | BG1NUC-HC261 |
| 708 | 2P | BG1NUC-HC262 |
| 709 | 2P | BG1NUC-HC263 |
| 710 | 2P | BG1NUC-HC264 |
| 711 | RBD | BG1NUC-HC265 |
| 712 | 2P | BG1NUC-HC266 |
| 713 | 2P | BG1NUC-HC267 |
| 714 | 2P | BG1NUC-HC268 |
| 715 | RBD | BG1NUC-HC269 |
| 716 | RBD | BG1NUC-HC270 |
| 717 | RBD | BG1NUC-HC271 |
| 718 | RBD | BG1NUC-HC272 |
| 719 | RBD | BG1NUC-HC273 |
| 720 | 2P | BG1NUC-HC274 |
| 721 | 2P | BG1NUC-HC275 |
| 722 | 2P | BG1NUC-HC276 |
| 723 | RBD | BG1NUC-HC277 |
| 724 | 2P | BG1NUC-HC278 |
| 725 | 2P | BG1NUC-HC279 |
| 726 | 2P | BG1NUC-HC280 |
| 727 | 2P | BG1NUC-HC281 |
| 728 | 2P | BG1NUC-HC282 |
| 729 | 2P | BG1NUC-HC283 |
| 730 | RBD | BG1NUC-HC284 |
| 731 | 2P | BG1NUC-HC285 |
| 732 | 2P | BG1NUC-HC286 |
| 733 | 2P | BG1NUC-HC287 |
| 734 | 2P | BG1NUC-HC288 |
| 735 | 2P | BG1NUC-HC289 |
| 736 | 2P | BG1NUC-HC290 |
| 737 | 2P | BG1NUC-HC291 |
| 738 | RBD | BG1NUC-HC292 |
| 739 | RBD | BG1NUC-HC293 |
| 740 | RBD | BG1NUC-HC294 |
| 741 | RBD | BG1NUC-HC295 |
| 742 | RBD | BG1NUC-HC296 |
| 743 | RBD | BG1NUC-HC297 |
| 744 | 2P | BG1NUC-HC298 |
| 745 | 2P | BG1NUC-HC299 |
| 746 | RBD | BG1NUC-HC300 |
| 747 | RBD | BG1NUC-HC301 |
| 748 | RBD | BG1NUC-HC302 |
| 749 | RBD | BG1NUC-HC303 |
| 750 | RBD | BG1NUC-HC304 |
| 751 | 2P | BG1NUC-HC305 |
| 752 | RBD | BG1NUC-HC306 |
| 753 | RBD | BG1NUC-HC307 |
| 754 | 2P | BG1NUC-HC308 |
| 755 | RBD | BG1NUC-LC1 |
| 756 | 2P | BG1NUC-LC2 |
| 757 | RBD | BG1NUC-LC3 |
| 758 | 2P | BG1NUC-LC4 |
| 759 | RBD | BG1NUC-LC5 |
| 760 | 2P | BG1NUC-LC6 |
| 761 | RBD | BG1NUC-LC7 |
| 762 | RBD | BG1NUC-LC8 |
| 763 | RBD | BG1NUC-LC9 |
| 764 | RBD | BG1NUC-LC10 |
| 765 | RBD | BG1NUC-LC11 |
| 766 | 2P | BG1NUC-LC12 |
| 767 | RBD | BG1NUC-LC13 |
| 768 | RBD | BG1NUC-LC14 |
| 769 | RBD | BG1NUC-LC15 |
| 770 | RBD | BG1NUC-LC16 |
| 771 | RBD | BG1NUC-LC17 |
| 772 | 2P | BG1NUC-LC18 |
| 773 | 2P | BG1NUC-LC19 |
| 774 | RBD | BG1NUC-LC20 |
| 775 | 2P | BG1NUC-LC21 |
| 776 | 2P | BG1NUC-LC22 |
| 777 | 2P | BG1NUC-LC23 |
| 778 | 2P | BG1NUC-LC24 |
| 779 | RBD | BG1NUC-LC25 |
| 780 | 2P | BG1NUC-LC26 |
| 781 | RBD | BG1NUC-LC27 |
| 782 | 2P | BG1NUC-LC28 |
| 783 | RBD | BG1NUC-LC29 |
| 784 | 2P | BG1NUC-LC30 |
| 785 | RBD | BG1NUC-LC31 |
| 786 | RBD | BG1NUC-LC32 |
| 787 | 2P | BG1NUC-LC33 |
| 788 | 2P | BG1NUC-LC34 |
| 789 | RBD | BG1NUC-LC35 |
| 790 | 2P | BG1NUC-LC36 |
| 791 | RBD | BG1NUC-LC37 |
| 792 | RBD | BG1NUC-LC38 |
| 793 | 2P | BG1NUC-LC39 |
| 794 | RBD | BG1NUC-LC40 |
| 795 | RBD | BG1NUC-LC41 |
| 796 | 2P | BG1NUC-LC42 |
| 797 | 2P | BG1NUC-LC43 |
| 798 | RBD | BG1NUC-LC44 |
| 799 | RBD | BG1NUC-LC45 |
| 800 | RBD | BG1NUC-LC46 |
| 801 | 2P | BG1NUC-LC47 |
| 802 | RBD | BG1NUC-LC48 |
| 803 | RBD | BG1NUC-LC49 |
| 804 | RBD | BG1NUC-LC50 |
| 805 | 2P | BG1NUC-LC51 |
| 806 | 2P | BG1NUC-LC52 |
| 807 | RBD | BG1NUC-LC53 |
| 808 | 2P | BG1NUC-LC54 |
| 809 | 2P | BG1NUC-LC55 |
| 810 | RBD | BG1NUC-LC56 |
| 811 | 2P | BG1NUC-LC57 |
| 812 | 2P | BG1NUC-LC58 |
| 813 | 2P | BG1NUC-LC59 |
| 814 | RBD | BG1NUC-LC60 |
| 815 | RBD | BG1NUC-LC61 |
| 816 | RBD | BG1NUC-LC62 |
| 817 | 2P | BG1NUC-LC63 |
| 818 | 2P | BG1NUC-LC64 |
| 819 | 2P | BG1NUC-LC65 |
| 820 | 2P | BG1NUC-LC66 |
| 821 | RBD | BG1NUC-LC67 |
| 822 | RBD | BG1NUC-LC68 |
| 823 | 2P | BG1NUC-LC69 |
| 824 | 2P | BG1NUC-LC70 |
| 825 | 2P | BG1NUC-LC71 |
| 826 | RBD | BG1NUC-LC72 |
| 827 | RBD | BG1NUC-LC73 |
| 828 | RBD | BG1NUC-LC74 |
| 829 | 2P | BG1NUC-LC75 |
| 830 | RBD | BG1NUC-LC76 |
| 831 | 2P | BG1NUC-LC77 |
| 832 | 2P | BG1NUC-LC78 |
| 833 | 2P | BG1NUC-LC79 |
| 834 | RBD | BG1NUC-LC80 |
| 835 | RBD | BG1NUC-LC81 |
| 836 | 2P | BG1NUC-LC82 |
| 837 | RBD | BG1NUC-LC83 |
| 838 | 2P | BG1NUC-LC84 |
| 839 | 2P | BG1NUC-LC85 |
| 840 | 2P | BG1NUC-LC86 |
| 841 | RBD | BG1NUC-LC87 |
| 842 | 2P | BG1NUC-LC88 |
| 843 | RBD | BG1NUC-LC89 |
| 844 | RBD | BG1NUC-LC90 |
| 845 | RBD | BG1NUC-LC91 |
| 846 | RBD | BG1NUC-LC92 |
| 847 | RBD | BG1NUC-LC93 |
| 848 | RBD | BG1NUC-LC94 |
| 849 | RBD | BG1NUC-LC95 |
| 850 | RBD | BG1NUC-LC96 |
| 851 | 2P | BG1NUC-LC97 |
| 852 | 2P | BG1NUC-LC98 |
| 853 | 2P | BG1NUC-LC99 |
| 854 | RBD | BG1NUC-LC100 |
| 855 | 2P | BG1NUC-LC101 |
| 856 | RBD | BG1NUC-LC102 |
| 857 | 2P | BG1NUC-LC103 |
| 858 | 2P | BG1NUC-LC104 |
| 859 | 2P | BG1NUC-LC105 |
| 860 | 2P | BG1NUC-LC106 |
| 861 | RBD | BG1NUC-LC107 |
| 862 | RBD | BG1NUC-LC108 |
| 863 | 2P | BG1NUC-LC109 |
| 864 | 2P | BG1NUC-LC110 |
| 865 | 2P | BG1NUC-LC111 |
| 866 | 2P | BG1NUC-LC112 |
| 867 | 2P | BG1NUC-LC113 |
| 868 | RBD | BG1NUC-LC114 |
| 869 | RBD | BG1NUC-LC115 |
| 870 | RBD | BG1NUC-LC116 |
| 871 | RBD | BG1NUC-LC117 |
| 872 | 2P | BG1NUC-LC118 |
| 873 | 2P | BG1NUC-LC119 |
| 874 | RBD | BG1NUC-LC120 |
| 875 | 2P | BG1NUC-LC121 |
| 876 | 2P | BG1NUC-LC122 |
| 877 | 2P | BG1NUC-LC123 |
| 878 | RBD | BG1NUC-LC124 |
| 879 | RBD | BG1NUC-LC125 |
| 880 | RBD | BG1NUC-LC126 |
| 881 | RBD | BG1NUC-LC127 |
| 882 | RBD | BG1NUC-LC128 |
| 883 | RBD | BG1NUC-LC129 |
| 884 | RBD | BG1NUC-LC130 |
| 885 | RBD | BG1NUC-LC131 |
| 886 | RBD | BG1NUC-LC132 |
| 887 | RBD | BG1NUC-LC133 |
| 888 | 2P | BG1NUC-LC134 |
| 889 | 2P | BG1NUC-LC135 |
| 890 | RBD | BG1NUC-LC136 |
| 891 | RBD | BG1NUC-LC137 |
| 892 | RBD | BG1NUC-LC138 |
| 893 | RBD | BG1NUC-LC139 |
| 894 | 2P | BG1NUC-LC140 |
| 895 | 2P | BG1NUC-LC141 |
| 896 | 2P | BG1NUC-LC142 |
| 897 | 2P | BG1NUC-LC143 |
| 898 | 2P | BG1NUC-LC144 |
| 899 | 2P | BG1NUC-LC145 |
| 900 | 2P | BG1NUC-LC146 |
| 901 | 2P | BG1NUC-LC147 |
| 902 | 2P | BG1NUC-LC148 |
| 903 | 2P | BG1NUC-LC149 |
| 904 | RBD | BG1NUC-LC150 |
| 905 | 2F | BG1NUC-LC151 |
| 906 | RBD | BG1NUC-LC152 |
| 907 | RBD | BG1NUC-LC153 |
| 908 | 2P | BG1NUC-LC154 |
| 909 | 2F | BG1NUC-LC155 |
| 910 | RBD | BG1NUC-LC156 |
| 911 | 2P | BG1NUC-LC157 |
| 912 | 2P | BG1NUC-LC158 |
| 913 | 2P | BG1NUC-LC159 |
| 914 | 2P | BG1NUC-LC160 |
| 915 | RBD | BG1NUC-LC161 |
| 916 | 2P | BG1NUC-LC162 |
| 917 | RBD | BG1NUC-LC163 |
| 918 | RBD | BG1NUC-LC164 |
| 919 | RBD | BG1NUC-LC165 |
| 920 | RBD | BG1NUC-LC166 |
| 921 | 2P | BG1NUC-LC167 |
| 922 | RBD | BG1NUC-LC168 |
| 923 | RBD | BG1NUC-LC169 |
| 924 | 2P | BG1NUC-LC170 |
| 925 | 2P | BG1NUC-LC171 |
| 926 | 2P | BG1NUC-LC172 |
| 927 | RBD | BG1NUC-LC173 |
| 928 | RBD | BG1NUC-LC174 |
| 929 | RBD | BG1NUC-LC175 |
| 930 | 2P | BG1NUC-LC176 |
| 931 | RBD | BG1NUC-LC177 |
| 932 | RBD | BG1NUC-LC178 |
| 933 | 2P | BG1NUC-LC179 |
| 934 | RBD | BG1NUC-LC180 |
| 935 | RBD | BG1NUC-LC181 |
| 936 | RBD | BG1NUC-LC182 |
| 937 | RBD | BG1NUC-LC183 |
| 938 | 2F | BG1NUC-LC184 |
| 939 | RBD | BG1NUC-LC185 |
| 940 | RBD | BG1NUC-LC186 |
| 941 | RBD | BG1NUC-LC187 |
| 942 | RBD | BG1NUC-LC188 |
| 943 | 2P | BG1NUC-LC189 |
| 944 | RBD | BG1NUC-LC190 |
| 945 | 2P | BG1NUC-LC191 |
| 946 | 2P | BG1NUC-LC192 |
| 947 | 2P | BG1NUC-LC193 |
| 948 | 2P | BG1NUC-LC194 |
| 949 | 2P | BG1NUC-LC195 |
| 950 | 2P | BG1NUC-LC196 |
| 951 | 2P | BG1NUC-LC197 |
| 952 | 2P | BG1NUC-LC198 |
| 953 | 2P | BG1NUC-LC199 |
| 954 | RBD | BG1NUC-LC200 |
| 955 | RBD | BG1NUC-LC201 |
| 956 | RBD | BG1NUC-LC202 |
| 957 | 2P | BG1NUC-LC203 |
| 958 | 2P | BG1NUC-LC204 |
| 959 | 2P | BG1NUC-LC205 |
| 960 | RBD | BG1NUC-LC206 |
| 961 | 2P | BG1NUC-LC207 |
| 962 | 2P | BG1NUC-LC208 |
| 963 | 2P | BG1NUC-LC209 |
| 964 | RBD | BG1NUC-LC210 |
| 965 | RBD | BG1NUC-LC211 |
| 966 | 2P | BG1NUC-LC212 |
| 967 | 2P | BG1NUC-LC213 |
| 968 | 2P | BG1NUC-LC214 |
| 969 | 2F | BG1NUC-LC215 |
| 970 | RBD | BG1NUC-LC216 |
| 971 | 2P | BG1NUC-LC217 |
| 972 | 2P | BG1NUC-LC218 |
| 973 | RBD | BG1NUC-LC219 |
| 974 | 2P | BG1NUC-LC220 |
| 975 | 2P | BG1NUC-LC221 |
| 976 | 2P | BG1NUC-LC222 |
| 977 | 2P | BG1NUC-LC223 |
| 978 | RBD | BG1NUC-LC224 |
| 979 | RBD | BG1NUC-LC225 |
| 980 | 2P | BG1NUC-LC226 |
| 981 | 2P | BG1NUC-LC227 |
| 982 | 2P | BG1NUC-LC228 |
| 983 | RBD | BG1NUC-LC229 |
| 984 | RBD | BG1NUC-LC230 |
| 985 | RBD | BG1NUC-LC231 |
| 986 | 2P | BG1NUC-LC232 |
| 987 | RBD | BG1NUC-LC233 |
| 988 | RBD | BG1NUC-LC234 |
| 989 | 2P | BG1NUC-LC235 |
| 990 | 2P | BG1NUC-LC236 |
| 991 | 2P | BG1NUC-LC237 |
| 992 | 2P | BG1NUC-LC238 |
| 993 | RBD | BG1NUC-LC239 |
| 994 | RBD | BG1NUC-LC240 |
| 995 | 2P | BG1NUC-LC241 |
| 996 | 2P | BG1NUC-LC242 |
| 997 | 2P | BG1NUC-LC243 |
| 998 | RBD | BG1NUC-LC244 |
| 999 | RBD | BG1NUC-LC245 |
| 1000 | RBD | BG1NUC-LC246 |
| 1001 | 2P | BG1NUC-LC247 |
| 1002 | 2P | BG1NUC-LC248 |
| 1003 | 2P | BG1NUC-LC249 |
| 1004 | RBD | BG1NUC-LC250 |
| 1005 | RBD | BG1NUC-LC251 |
| 1006 | 2P | BG1NUC-LC252 |
| 1007 | RBD | BG1NUC-LC253 |
| 1008 | RBD | BG1NUC-LC254 |
| 1009 | RBD | BG1NUC-LC255 |
| 1010 | 2P | BG1NUC-LC256 |
| 1011 | 2P | BG1NUC-LC257 |
| 1012 | 2P | BG1NUC-LC258 |
| 1013 | 2P | BG1NUC-LC259 |
| 1014 | RBD | BG1NUC-LC260 |
| 1015 | RBD | BG1NUC-LC261 |
| 1016 | 2P | BG1NUC-LC262 |
| 1017 | 2P | BG1NUC-LC263 |
| 1018 | 2P | BG1NUC-LC264 |
| 1019 | RBD | BG1NUC-LC265 |
| 1020 | 2P | BG1NUC-LC266 |
| 1021 | 2P | BG1NUC-LC267 |
| 1022 | 2P | BG1NUC-LC268 |
| 1023 | RBD | BG1NUC-LC269 |
| 1024 | RBD | BG1NUC-LC270 |
| 1025 | RBD | BG1NUC-LC271 |
| 1026 | RBD | BG1NUC-LC272 |
| 1027 | RBD | BG1NUC-LC273 |
| 1028 | 2P | BG1NUC-LC274 |
| 1029 | 2P | BG1NUC-LC275 |
| 1030 | 2P | BG1NUC-LC276 |
| 1031 | RBD | BG1NUC-LC277 |
| 1032 | 2P | BG1NUC-LC278 |
| 1033 | 2P | BG1NUC-LC279 |
| 1034 | 2P | BG1NUC-LC280 |
| 1035 | 2P | BG1NUC-LC281 |
| 1036 | 2P | BG1NUC-LC282 |
| 1037 | 2P | BG1NUC-LC283 |
| 1038 | RBD | BG1NUC-LC284 |
| 1039 | 2P | BG1NUC-LC285 |
| 1040 | 2P | BG1NUC-LC286 |
| 1041 | 2P | BG1NUC-LC287 |
| 1042 | 2P | BG1NUC-LC288 |
| 1043 | 2P | BG1NUC-LC289 |
| 1044 | 2P | BG1NUC-LC290 |
| 1045 | 2P | BG1NUC-LC291 |
| 1046 | RBD | BG1NUC-LC292 |
| 1047 | RBD | BG1NUC-LC293 |
| 1048 | RBD | BG1NUC-LC294 |
| 1049 | RBD | BG1NUC-LC295 |
| 1050 | RBD | BG1NUC-LC296 |
| 1051 | RBD | BG1NUC-LC297 |
| 1052 | 2P | BG1NUC-LC298 |
| 1053 | 2P | BG1NUC-LC299 |
| 1054 | RBD | BG1NUC-LC300 |
| 1055 | RBD | BG1NUC-LC301 |
| 1056 | RBD | BG1NUC-LC302 |
| 1057 | RBD | BG1NUC-LC303 |
| 1058 | RBD | BG1NUC-LC304 |
| 1059 | 2P | BG1NUC-LC305 |
| 1060 | RBD | BG1NUC-LC306 |
| 1061 | RBD | BG1NUC-LC307 |
| 1062 | 2P | BG1NUC-LC308 |
| 1063 | RBD | BG2NUC-HC1 |
| 1064 | RBD | BG2NUC-HC2 |
| 1065 | 2P | BG2NUC-HC3 |
| 1066 | RBD | BG2NUC-HC4 |
| 1067 | RBD | BG2NUC-HC5 |
| 1068 | RBD | BG2NUC-HC6 |
| 1069 | RBD | BG2NUC-HC7 |
| 1070 | RBD | BG2NUC-HC8 |
| 1071 | 2P | BG2NUC-HC9 |
| 1072 | RBD | BG2NUC-HC10 |
| 1073 | 2P | BG2NUC-HC11 |
| 1074 | RBD | BG2NUC-HC12 |
| 1075 | RBD | BG2NUC-HC13 |
| 1076 | RBD | BG2NUC-HC14 |
| 1077 | RBD | BG2NUC-HC15 |
| 1078 | RBD | BG2NUC-HC16 |
| 1079 | RBD | BG2NUC-HC17 |
| 1080 | RBD | BG2NUC-HC18 |
| 1081 | RBD | BG2NUC-HC19 |
| 1082 | RBD | BG2NUC-HC20 |
| 1083 | RBD | BG2NUC-HC21 |
| 1084 | 2P | BG2NUC-HC22 |
| 1085 | RBD | BG2NUC-HC23 |
| 1086 | RBD | BG2NUC-HC24 |
| 1087 | RBD | BG2NUC-HC25 |
| 1088 | RBD | BG2NUC-HC26 |
| 1089 | RBD | BG2NUC-HC27 |
| 1090 | RBD | BG2NUC-HC28 |
| 1091 | RBD | BG2NUC-HC29 |
| 1092 | 2P | BG2NUC-HC30 |
| 1093 | RBD | BG2NUC-HC31 |
| 1094 | RBD | BG2NUC-HC32 |
| 1095 | RBD | BG2NUC-HC33 |
| 1096 | RBD | BG2NUC-HC34 |
| 1097 | 2P | BG2NUC-HC35 |
| 1098 | RBD | BG2NUC-HC36 |
| 1099 | 2P | BG2NUC-HC37 |
| 1100 | RBD | BG2NUC-HC38 |
| 1101 | RBD | BG2NUC-HC39 |
| 1102 | RBD | BG2NUC-HC40 |
| 1103 | RBD | BG2NUC-HC41 |
| 1104 | RBD | BG2NUC-HC42 |
| 1105 | RBD | BG2NUC-HC43 |
| 1106 | RBD | BG2NUC-HC44 |
| 1107 | RBD | BG2NUC-HC45 |
| 1108 | RBD | BG2NUC-HC46 |
| 1109 | 2P | BG2NUC-HC47 |
| 1110 | 2P | BG2NUC-HC48 |
| 1111 | RBD | BG2NUC-HC49 |
| 1112 | RBD | BG2NUC-HC50 |
| 1113 | 2P | BG2NUC-HC51 |
| 1114 | RBD | BG2NUC-HC52 |
| 1115 | RBD | BG2NUC-HC53 |
| 1116 | 2P | BG2NUC-HC54 |
| 1117 | RBD | BG2NUC-HC55 |
| 1118 | RBD | BG2NUC-HC56 |
| 1119 | 2P | BG2NUC-HC57 |
| 1120 | RBD | BG2NUC-HC58 |
| 1121 | 2P | BG2NUC-HC59 |
| 1122 | RBD | BG2NUC-HC60 |
| 1123 | RBD | BG2NUC-HC61 |
| 1124 | 2P | BG2NUC-HC62 |
| 1125 | 2F | BG2NUC-HC63 |
| 1126 | 2F | BG2NUC-HC64 |
| 1127 | 2P | BG2NUC-HC65 |
| 1128 | 2P | BG2NUC-HC66 |
| 1129 | 2P | BG2NUC-HC67 |
| 1130 | 2P | BG2NUC-HC68 |
| 1131 | 2P | BG2NUC-HC69 |
| 1132 | 2P | BG2NUC-HC70 |
| 1133 | RBD | BG2NUC-HC71 |
| 1134 | RBD | BG2NUC-HC72 |
| 1135 | RBD | BG2NUC-HC73 |
| 1136 | 2F | BG2NUC-HC74 |
| 1137 | RBD | BG2NUC-HC75 |
| 1138 | RBD | BG2NUC-HC76 |
| 1139 | 2P | BG2NUC-HC77 |
| 1140 | 2P | BG2NUC-HC78 |
| 1141 | RBD | BG2NUC-HC79 |
| 1142 | 2P | BG2NUC-HC80 |
| 1143 | RBD | BG2NUC-HC81 |
| 1144 | 2P | BG2NUC-HC82 |
| 1145 | 2P | BG2NUC-HC83 |
| 1146 | RBD | BG2NUC-HC84 |
| 1147 | RBD | BG2NUC-HC85 |
| 1148 | 2P | BG2NUC-HC86 |
| 1149 | RBD | BG2NUC-HC87 |
| 1150 | 2P | BG2NUC-HC88 |
| 1151 | RBD | BG2NUC-HC89 |
| 1152 | RBD | BG2NUC-HC90 |
| 1153 | 2P | BG2NUC-HC91 |
| 1154 | 2P | BG2NUC-HC92 |
| 1155 | 2P | BG2NUC-HC93 |
| 1156 | 2F | BG2NUC-HC94 |
| 1157 | 2P | BG2NUC-HC95 |
| 1158 | 2P | BG2NUC-HC96 |
| 1159 | RBD | BG2NUC-HC97 |
| 1160 | 2P | BG2NUC-HC98 |
| 1161 | 2P | BG2NUC-HC99 |
| 1162 | RBD | BG2NUC-HC100 |
| 1163 | RBD | BG2NUC-HC101 |
| 1164 | RBD | BG2NUC-HC102 |
| 1165 | RBD | BG2NUC-HC103 |
| 1166 | RBD | BG2NUC-HC104 |
| 1167 | 2P | BG2NUC-HC105 |
| 1168 | RBD | BG2NUC-HC106 |
| 1169 | RBD | BG2NUC-HC107 |
| 1170 | RBD | BG2NUC-HC108 |
| 1171 | 2P | BG2NUC-HC109 |
| 1172 | RBD | BG2NUC-HC110 |
| 1173 | RBD | BG2NUC-HC111 |
| 1174 | RBD | BG2NUC-HC112 |
| 1175 | 2P | BG2NUC-HC113 |
| 1176 | 2P | BG2NUC-HC114 |
| 1177 | RBD | BG2NUC-HC115 |
| 1178 | RBD | BG2NUC-HC116 |
| 1179 | 2P | BG2NUC-HC117 |
| 1180 | 2P | BG2NUC-HC118 |
| 1181 | 2F | BG2NUC-HC119 |
| 1182 | 2F | BG2NUC-HC120 |
| 1183 | RBD | BG2NUC-HC121 |
| 1184 | RBD | BG2NUC-HC122 |
| 1185 | 2P | BG2NUC-HC123 |
| 1186 | 2P | BG2NUC-HC124 |
| 1187 | RBD | BG2NUC-HC125 |
| 1188 | RBD | BG2NUC-HC126 |
| 1189 | 2P | BG2NUC-HC127 |
| 1190 | 2P | BG2NUC-HC128 |
| 1191 | 2P | BG2NUC-HC129 |
| 1192 | 2P | BG2NUC-HC130 |
| 1193 | 2P | BG2NUC-HC131 |
| 1194 | 2P | BG2NUC-HC132 |
| 1195 | 2P | BG2NUC-HC133 |
| 1196 | 2P | BG2NUC-HC134 |
| 1197 | RBD | BG2NUC-HC135 |
| 1198 | RBD | BG2NUC-HC136 |
| 1199 | RBD | BG2NUC-HC137 |
| 1200 | 2P | BG2NUC-HC138 |
| 1201 | 2P | BG2NUC-HC139 |
| 1202 | 2P | BG2NUC-HC140 |
| 1203 | 2P | BG2NUC-HC141 |
| 1204 | 2P | BG2NUC-HC142 |
| 1205 | 2P | BG2NUC-HC143 |
| 1206 | 2P | BG2NUC-HC144 |
| 1207 | RBD | BG2NUC-HC145 |
| 1208 | RBD | BG2NUC-HC146 |
| 1209 | 2P | BG2NUC-HC147 |
| 1210 | RBD | BG2NUC-HC148 |
| 1211 | 2P | BG2NUC-HC149 |
| 1212 | 2P | BG2NUC-HC150 |
| 1213 | 2P | BG2NUC-HC151 |
| 1214 | 2P | BG2NUC-HC152 |
| 1215 | 2P | BG2NUC-HC153 |
| 1216 | 2P | BG2NUC-HC154 |
| 1217 | 2P | BG2NUC-HC155 |
| 1218 | 2P | BG2NUC-HC156 |
| 1219 | RBD | BG2NUC-HC157 |
| 1220 | 2P | BG2NUC-HC158 |
| 1221 | 2P | BG2NUC-HC159 |
| 1222 | RBD | BG2NUC-HC160 |
| 1223 | 2P | BG2NUC-HC161 |
| 1224 | 2P | BG2NUC-HC162 |
| 1225 | RBD | BG2NUC-HC163 |
| 1226 | 2P | BG2NUC-HC164 |
| 1227 | RBD | BG2NUC-HC165 |
| 1228 | RBD | BG2NUC-HC166 |
| 1229 | 2P | BG2NUC-HC167 |
| 1230 | 2P | BG2NUC-HC168 |
| 1231 | 2P | BG2NUC-HC169 |
| 1232 | 2P | BG2NUC-HC170 |
| 1233 | RBD | BG2NUC-HC171 |
| 1234 | 2P | BG2NUC-HC172 |
| 1235 | 2P | BG2NUC-HC173 |
| 1236 | 2P | BG2NUC-HC174 |
| 1237 | RBD | BG2NUC-HC175 |
| 1238 | 2P | BG2NUC-HC176 |
| 1239 | RBD | BG2NUC-HC177 |
| 1240 | RBD | BG2NUC-HC178 |
| 1241 | RBD | BG2NUC-HC179 |
| 1242 | 2P | BG2NUC-HC180 |
| 1243 | RBD | BG2NUC-HC181 |
| 1244 | RBD | BG2NUC-HC182 |
| 1245 | 2P | BG2NUC-HC183 |
| 1246 | 2P | BG2NUC-HC184 |
| 1247 | RBD | BG2NUC-HC185 |
| 1248 | 2P | BG2NUC-HC186 |
| 1249 | RBD | BG2NUC-HC187 |
| 1250 | 2P | BG2NUC-HC188 |
| 1251 | 2P | BG2NUC-HC189 |
| 1252 | 2P | BG2NUC-HC190 |
| 1253 | 2P | BG2NUC-HC191 |
| 1254 | RBD | BG2NUC-HC192 |
| 1255 | 2P | BG2NUC-HC193 |
| 1256 | RBD | BG2NUC-HC194 |
| 1257 | 2P | BG2NUC-HC195 |
| 1258 | 2P | BG2NUC-HC196 |
| 1259 | RBD | BG2NUC-HC197 |
| 1260 | RBD | BG2NUC-HC198 |
| 1261 | 2P | BG2NUC-HC199 |
| 1262 | 2P | BG2NUC-HC200 |
| 1263 | RBD | BG2NUC-HC201 |
| 1264 | 2P | BG2NUC-HC202 |
| 1265 | RBD | BG2NUC-HC203 |
| 1266 | 2P | BG2NUC-HC204 |
| 1267 | RBD | BG2NUC-HC205 |
| 1268 | 2P | BG2NUC-HC206 |
| 1269 | RBD | BG2NUC-HC207 |
| 1270 | 2P | BG2NUC-HC208 |
| 1271 | 2P | BG2NUC-HC209 |
| 1272 | 2P | BG2NUC-HC210 |
| 1273 | RBD | BG2NUC-HC211 |
| 1274 | 2P | BG2NUC-HC212 |
| 1275 | RBD | BG2NUC-HC213 |
| 1276 | 2P | BG2NUC-HC214 |
| 1277 | RBD | BG2NUC-HC215 |
| 1278 | 2F | BG2NUC-HC216 |
| 1279 | RBD | BG2NUC-HC217 |
| 1280 | RBD | BG2NUC-HC218 |
| 1281 | RBD | BG2NUC-HC219 |
| 1282 | 2P | BG2NUC-HC220 |
| 1283 | RBD | BG2NUC-HC221 |
| 1284 | RBD | BG2NUC-HC222 |
| 1285 | RBD | BG2NUC-HC223 |
| 1286 | 2P | BG2NUC-HC224 |
| 1287 | RBD | BG2NUC-HC225 |
| 1288 | 2P | BG2NUC-HC226 |
| 1289 | 2P | BG2NUC-HC227 |
| 1290 | 2P | BG2NUC-HC228 |
| 1291 | RBD | BG2NUC-HC229 |
| 1292 | 2P | BG2NUC-HC230 |
| 1293 | RBD | BG2NUC-HC231 |
| 1294 | 2P | BG2NUC-HC232 |
| 1295 | 2P | BG2NUC-HC233 |
| 1296 | 2P | BG2NUC-HC234 |
| 1297 | RBD | BG2NUC-HC235 |
| 1298 | RBD | BG2NUC-HC236 |
| 1299 | 2P | BG2NUC-HC237 |
| 1300 | RBD | BG2NUC-HC238 |
| 1301 | 2P | BG2NUC-HC239 |
| 1302 | 2P | BG2NUC-HC240 |
| 1303 | 2P | BG2NUC-HC241 |
| 1304 | RBD | BG2NUC-HC242 |
| 1305 | 2P | BG2NUC-HC243 |
| 1306 | RBD | BG2NUC-HC244 |
| 1307 | RBD | BG2NUC-HC245 |
| 1308 | 2P | BG2NUC-HC246 |
| 1309 | RBD | BG2NUC-HC247 |
| 1310 | RBD | BG2NUC-HC248 |
| 1311 | 2P | BG2NUC-HC249 |
| 1312 | 2P | BG2NUC-HC250 |
| 1313 | 2P | BG2NUC-HC251 |
| 1314 | RBD | BG2NUC-HC252 |
| 1315 | 2P | BG2NUC-HC253 |
| 1316 | RBD | BG2NUC-HC254 |
| 1317 | 2P | BG2NUC-HC255 |
| 1318 | RBD | BG2NUC-HC256 |
| 1319 | 2P | BG2NUC-HC257 |
| 1320 | 2P | BG2NUC-HC258 |
| 1321 | 2P | BG2NUC-HC259 |
| 1322 | 2P | BG2NUC-HC260 |
| 1323 | 2P | BG2NUC-HC261 |
| 1324 | RBD | BG2NUC-HC262 |
| 1325 | 2P | BG2NUC-HC263 |
| 1326 | RBD | BG2NUC-HC264 |
| 1327 | 2P | BG2NUC-HC265 |
| 1328 | 2P | BG2NUC-HC266 |
| 1329 | 2P | BG2NUC-HC267 |
| 1330 | 2P | BG2NUC-HC268 |
| 1331 | RBD | BG2NUC-HC269 |
| 1332 | 2P | BG2NUC-HC270 |
| 1333 | 2P | BG2NUC-HC271 |
| 1334 | 2P | BG2NUC-HC272 |
| 1335 | 2P | BG2NUC-HC273 |
| 1336 | 2P | BG2NUC-HC274 |
| 1337 | 2P | BG2NUC-HC275 |
| 1338 | 2P | BG2NUC-HC276 |
| 1339 | 2P | BG2NUC-HC277 |
| 1340 | 2P | BG2NUC-HC278 |
| 1341 | 2P | BG2NUC-HC279 |
| 1342 | RBD | BG2NUC-HC280 |
| 1343 | RBD | BG2NUC-HC281 |
| 1344 | RBD | BG2NUC-HC282 |
| 1345 | 2P | BG2NUC-HC283 |
| 1346 | 2P | BG2NUC-HC284 |
| 1347 | 2P | BG2NUC-HC285 |
| 1348 | 2P | BG2NUC-HC286 |
| 1349 | RBD | BG2NUC-HC287 |
| 1350 | 2P | BG2NUC-HC288 |
| 1351 | 2P | BG2NUC-HC289 |
| 1352 | RBD | BG2NUC-HC290 |
| 1353 | 2P | BG2NUC-HC291 |
| 1354 | 2P | BG2NUC-HC292 |
| 1355 | 2P | BG2NUC-HC293 |
| 1356 | 2P | BG2NUC-HC294 |
| 1357 | RBD | BG2NUC-HC295 |
| 1358 | 2P | BG2NUC-HC296 |
| 1359 | 2P | BG2NUC-HC297 |
| 1360 | RBD | BG2NUC-HC298 |
| 1361 | 2P | BG2NUC-HC299 |
| 1362 | 2P | BG2NUC-HC300 |
| 1363 | 2P | BG2NUC-HC301 |
| 1364 | 2P | BG2NUC-HC302 |
| 1365 | 2P | BG2NUC-HC303 |
| 1366 | 2P | BG2NUC-HC304 |
| 1367 | RBD | BG2NUC-HC305 |
| 1368 | 2P | BG2NUC-HC306 |
| 1369 | 2P | BG2NUC-HC307 |
| 1370 | 2P | BG2NUC-HC308 |
| 1371 | 2P | BG2NUC-HC309 |
| 1372 | RBD | BG2NUC-HC310 |
| 1373 | RBD | BG2NUC-HC311 |
| 1374 | RBD | BG2NUC-HC312 |
| 1375 | RBD | BG2NUC-HC313 |
| 1376 | RBD | BG2NUC-HC314 |
| 1377 | RBD | BG2NUC-HC315 |
| 1378 | 2P | BG2NUC-HC316 |
| 1379 | RBD | BG2NUC-HC317 |
| 1380 | 2P | BG2NUC-HC318 |
| 1381 | 2P | BG2NUC-HC319 |
| 1382 | 2P | BG2NUC-HC320 |
| 1383 | 2P | BG2NUC-HC321 |
| 1384 | RBD | BG2NUC-HC322 |
| 1385 | RBD | BG2NUC-HC323 |
| 1386 | RBD | BG2NUC-HC324 |
| 1387 | RBD | BG2NUC-HC325 |
| 1388 | RBD | BG2NUC-HC326 |
| 1389 | 2P | BG2NUC-HC327 |
| 1390 | 2P | BG2NUC-HC328 |
| 1391 | 2P | BG2NUC-HC329 |
| 1392 | 2P | BG2NUC-HC330 |
| 1393 | 2P | BG2NUC-HC331 |
| 1394 | 2P | BG2NUC-HC332 |
| 1395 | 2P | BG2NUC-HC333 |
| 1396 | RBD | BG2NUC-HC334 |
| 1397 | 2P | BG2NUC-HC335 |
| 1398 | 2P | BG2NUC-HC336 |
| 1399 | RBD | BG2NUC-HC337 |
| 1400 | 2P | BG2NUC-HC338 |
| 1401 | 2P | BG2NUC-HC339 |
| 1402 | 2P | BG2NUC-HC340 |
| 1403 | 2P | BG2NUC-HC341 |
| 1404 | 2P | BG2NUC-HC342 |
| 1405 | 2P | BG2NUC-HC343 |
| 1406 | 2P | BG2NUC-HC344 |
| 1407 | RBD | BG2NUC-HC345 |
| 1408 | 2P | BG2NUC-HC346 |
| 1409 | 2P | BG2NUC-HC347 |
| 1410 | 2P | BG2NUC-HC348 |
| 1411 | RBD | BG2NUC-HC349 |
| 1412 | RBD | BG2NUC-HC350 |
| 1413 | 2P | BG2NUC-HC351 |
| 1414 | RBD | BG2NUC-HC352 |
| 1415 | RBD | BG2NUC-HC353 |
| 1416 | 2P | BG2NUC-HC354 |
| 1417 | 2P | BG2NUC-HC355 |
| 1418 | 2P | BG2NUC-HC356 |
| 1419 | 2P | BG2NUC-HC357 |
| 1420 | 2P | BG2NUC-HC358 |
| 1421 | 2P | BG2NUC-HC359 |
| 1422 | 2P | BG2NUC-HC360 |
| 1423 | 2P | BG2NUC-HC361 |
| 1424 | 2P | BG2NUC-HC362 |
| 1425 | 2P | BG2NUC-HC363 |
| 1426 | RBD | BG2NUC-HC364 |
| 1427 | 2P | BG2NUC-HC365 |
| 1428 | 2P | BG2NUC-HC366 |
| 1429 | 2P | BG2NUC-HC367 |
| 1430 | RBD | BG2NUC-HC368 |
| 1431 | 2P | BG2NUC-HC369 |
| 1432 | 2P | BG2NUC-HC370 |
| 1433 | 2P | BG2NUC-HC371 |
| 1434 | 2P | BG2NUC-HC372 |
| 1435 | 2P | BG2NUC-HC373 |
| 1436 | 2P | BG2NUC-HC374 |
| 1437 | 2P | BG2NUC-HC375 |
| 1438 | 2P | BG2NUC-HC376 |
| 1439 | 2P | BG2NUC-HC377 |
| 1440 | 2P | BG2NUC-HC378 |
| 1441 | 2P | BG2NUC-HC379 |
| 1442 | 2P | BG2NUC-HC380 |
| 1443 | RBD | BG2NUC-LC1 |
| 1444 | RBD | BG2NUC-LC2 |
| 1445 | 2P | BG2NUC-LC3 |
| 1446 | RBD | BG2NUC-LC4 |
| 1447 | RBD | BG2NUC-LC5 |
| 1448 | RBD | BG2NUC-LC6 |
| 1449 | RBD | BG2NUC-LC7 |
| 1450 | RBD | BG2NUC-LC8 |
| 1451 | 2P | BG2NUC-LC9 |
| 1452 | RBD | BG2NUC-LC10 |
| 1453 | 2P | BG2NUC-LC11 |
| 1454 | RBD | BG2NUC-LC12 |
| 1455 | RBD | BG2NUC-LC13 |
| 1456 | RBD | BG2NUC-LC14 |
| 1457 | RBD | BG2NUC-LC15 |
| 1458 | RBD | BG2NUC-LC16 |
| 1459 | RBD | BG2NUC-LC17 |
| 1460 | RBD | BG2NUC-LC18 |
| 1461 | RBD | BG2NUC-LC19 |
| 1462 | RBD | BG2NUC-LC20 |
| 1463 | RBD | BG2NUC-LC21 |
| 1464 | 2P | BG2NUC-LC22 |
| 1465 | RBD | BG2NUC-LC23 |
| 1466 | RBD | BG2NUC-LC24 |
| 1467 | RBD | BG2NUC-LC25 |
| 1468 | RBD | BG2NUC-LC26 |
| 1469 | RBD | BG2NUC-LC27 |
| 1470 | RBD | BG2NUC-LC28 |
| 1471 | RBD | BG2NUC-LC29 |
| 1472 | 2P | BG2NUC-LC30 |
| 1473 | RBD | BG2NUC-LC31 |
| 1474 | RBD | BG2NUC-LC32 |
| 1475 | RBD | BG2NUC-LC33 |
| 1476 | RBD | BG2NUC-LC34 |
| 1477 | 2P | BG2NUC-LC35 |
| 1478 | RBD | BG2NUC-LC36 |
| 1479 | 2P | BG2NUC-LC37 |
| 1480 | RBD | BG2NUC-LC38 |
| 1481 | RBD | BG2NUC-LC39 |
| 1482 | RBD | BG2NUC-LC40 |
| 1483 | RBD | BG2NUC-LC41 |
| 1484 | RBD | BG2NUC-LC42 |
| 1485 | RBD | BG2NUC-LC43 |
| 1486 | RBD | BG2NUC-LC44 |
| 1487 | RBD | BG2NUC-LC45 |
| 1488 | RBD | BG2NUC-LC46 |
| 1489 | 2P | BG2NUC-LC47 |
| 1490 | 2P | BG2NUC-LC48 |
| 1491 | RBD | BG2NUC-LC49 |
| 1492 | RBD | BG2NUC-LC50 |
| 1493 | 2P | BG2NUC-LC51 |
| 1494 | RBD | BG2NUC-LC52 |
| 1495 | RBD | BG2NUC-LC53 |
| 1496 | 2P | BG2NUC-LC54 |
| 1497 | RBD | BG2NUC-LC55 |
| 1498 | RBD | BG2NUC-LC56 |
| 1499 | 2P | BG2NUC-LC57 |
| 1500 | RBD | BG2NUC-LC58 |
| 1501 | 2P | BG2NUC-LC59 |
| 1502 | RBD | BG2NUC-LC60 |
| 1503 | RBD | BG2NUC-LC61 |
| 1504 | 2P | BG2NUC-LC62 |
| 1505 | 2P | BG2NUC-LC63 |
| 1506 | 2P | BG2NUC-LC64 |
| 1507 | 2P | BG2NUC-LC65 |
| 1508 | 2P | BG2NUC-LC66 |
| 1509 | 2P | BG2NUC-LC67 |
| 1510 | 2P | BG2NUC-LC68 |
| 1511 | 2P | BG2NUC-LC69 |
| 1512 | 2P | BG2NUC-LC70 |
| 1513 | RBD | BG2NUC-LC71 |
| 1514 | RBD | BG2NUC-LC72 |
| 1515 | RBD | BG2NUC-LC73 |
| 1516 | 2P | BG2NUC-LC74 |
| 1517 | RBD | BG2NUC-LC75 |
| 1518 | RBD | BG2NUC-LC76 |
| 1519 | 2P | BG2NUC-LC77 |
| 1520 | 2P | BG2NUC-LC78 |
| 1521 | RBD | BG2NUC-LC79 |
| 1522 | 2P | BG2NUC-LC80 |
| 1523 | RBD | BG2NUC-LC81 |
| 1524 | 2P | BG2NUC-LC82 |
| 1525 | 2P | BG2NUC-LC83 |
| 1526 | RBD | BG2NUC-LC84 |
| 1527 | RBD | BG2NUC-LC85 |
| 1528 | 2P | BG2NUC-LC86 |
| 1529 | RBD | BG2NUC-LC87 |
| 1530 | 2P | BG2NUC-LC88 |
| 1531 | RBD | BG2NUC-LC89 |
| 1532 | RBD | BG2NUC-LC90 |
| 1533 | 2P | BG2NUC-LC91 |
| 1534 | 2P | BG2NUC-LC92 |
| 1535 | 2P | BG2NUC-LC93 |
| 1536 | 2P | BG2NUC-LC94 |
| 1537 | 2P | BG2NUC-LC95 |
| 1538 | 2P | BG2NUC-LC96 |
| 1539 | RBD | BG2NUC-LC97 |
| 1540 | 2P | BG2NUC-LC98 |
| 1541 | 2P | BG2NUC-LC99 |
| 1542 | RBD | BG2NUC-LC100 |
| 1543 | RBD | BG2NUC-LC101 |
| 1544 | RBD | BG2NUC-LC102 |
| 1545 | RBD | BG2NUC-LC103 |
| 1546 | RBD | BG2NUC-LC104 |
| 1547 | 2P | BG2NUC-LC105 |
| 1548 | RBD | BG2NUC-LC106 |
| 1549 | RBD | BG2NUC-LC107 |
| 1550 | RBD | BG2NUC-LC108 |
| 1551 | 2P | BG2NUC-LC109 |
| 1552 | RBD | BG2NUC-LC110 |
| 1553 | RBD | BG2NUC-LC111 |
| 1554 | RBD | BG2NUC-LC112 |
| 1555 | 2P | BG2NUC-LC113 |
| 1556 | 2P | BG2NUC-LC114 |
| 1557 | RBD | BG2NUC-LC115 |
| 1558 | RBD | BG2NUC-LC116 |
| 1559 | 2P | BG2NUC-LC117 |
| 1560 | 2P | BG2NUC-LC118 |
| 1561 | 2P | BG2NUC-LC119 |
| 1562 | 2P | BG2NUC-LC120 |
| 1563 | RBD | BG2NUC-LC121 |
| 1564 | RBD | BG2NUC-LC122 |
| 1565 | 2P | BG2NUC-LC123 |
| 1566 | 2P | BG2NUC-LC124 |
| 1567 | RBD | BG2NUC-LC125 |
| 1568 | RBD | BG2NUC-LC126 |
| 1569 | 2P | BG2NUC-LC127 |
| 1570 | 2P | BG2NUC-LC128 |
| 1571 | 2P | BG2NUC-LC129 |
| 1572 | 2P | BG2NUC-LC130 |
| 1573 | 2P | BG2NUC-LC131 |
| 1574 | 2P | BG2NUC-LC132 |
| 1575 | 2P | BG2NUC-LC133 |
| 1576 | 2P | BG2NUC-LC134 |
| 1577 | RBD | BG2NUC-LC135 |
| 1578 | RBD | BG2NUC-LC136 |
| 1579 | RBD | BG2NUC-LC137 |
| 1580 | 2P | BG2NUC-LC138 |
| 1581 | 2P | BG2NUC-LC139 |
| 1582 | 2P | BG2NUC-LC140 |
| 1583 | 2P | BG2NUC-LC141 |
| 1584 | 2P | BG2NUC-LC142 |
| 1585 | 2P | BG2NUC-LC143 |
| 1586 | 2P | BG2NUC-LC144 |
| 1587 | RBD | BG2NUC-LC145 |
| 1588 | RBD | BG2NUC-LC146 |
| 1589 | 2P | BG2NUC-LC147 |
| 1590 | RBD | BG2NUC-LC148 |
| 1591 | 2P | BG2NUC-LC149 |
| 1592 | 2P | BG2NUC-LC150 |
| 1593 | 2P | BG2NUC-LC151 |
| 1594 | 2P | BG2NUC-LC152 |
| 1595 | 2P | BG2NUC-LC153 |
| 1596 | 2P | BG2NUC-LC154 |
| 1597 | 2P | BG2NUC-LC155 |
| 1598 | 2P | BG2NUC-LC156 |
| 1599 | RBD | BG2NUC-LC157 |
| 1600 | 2P | BG2NUC-LC158 |
| 1601 | 2P | BG2NUC-LC159 |
| 1602 | RBD | BG2NUC-LC160 |
| 1603 | 2P | BG2NUC-LC161 |
| 1604 | 2P | BG2NUC-LC162 |
| 1605 | RBD | BG2NUC-LC163 |
| 1606 | 2P | BG2NUC-LC164 |
| 1607 | RBD | BG2NUC-LC165 |
| 1608 | RBD | BG2NUC-LC166 |
| 1609 | 2P | BG2NUC-LC167 |
| 1610 | 2P | BG2NUC-LC168 |
| 1611 | 2P | BG2NUC-LC169 |
| 1612 | 2P | BG2NUC-LC170 |
| 1613 | RBD | BG2NUC-LC171 |
| 1614 | 2P | BG2NUC-LC172 |
| 1615 | 2P | BG2NUC-LC173 |
| 1616 | 2P | BG2NUC-LC174 |
| 1617 | RBD | BG2NUC-LC175 |
| 1618 | 2P | BG2NUC-LC176 |
| 1619 | RBD | BG2NUC-LC177 |
| 1620 | RBD | BG2NUC-LC178 |
| 1621 | RBD | BG2NUC-LC179 |
| 1622 | 2P | BG2NUC-LC180 |
| 1623 | RBD | BG2NUC-LC181 |
| 1624 | RBD | BG2NUC-LC182 |
| 1625 | 2P | BG2NUC-LC183 |
| 1626 | 2P | BG2NUC-LC184 |
| 1627 | RBD | BG2NUC-LC185 |
| 1628 | 2P | BG2NUC-LC186 |
| 1629 | RBD | BG2NUC-LC187 |
| 1630 | 2P | BG2NUC-LC188 |
| 1631 | 2P | BG2NUC-LC189 |
| 1632 | 2P | BG2NUC-LC190 |
| 1633 | 2P | BG2NUC-LC191 |
| 1634 | RBD | BG2NUC-LC192 |
| 1635 | 2P | BG2NUC-LC193 |
| 1636 | RBD | BG2NUC-LC194 |
| 1637 | 2P | BG2NUC-LC195 |
| 1638 | 2P | BG2NUC-LC196 |
| 1639 | RBD | BG2NUC-LC197 |
| 1640 | RBD | BG2NUC-LC198 |
| 1641 | 2P | BG2NUC-LC199 |
| 1642 | 2P | BG2NUC-LC200 |
| 1643 | RBD | BG2NUC-LC201 |
| 1644 | 2P | BG2NUC-LC202 |
| 1645 | RBD | BG2NUC-LC203 |
| 1646 | 2P | BG2NUC-LC204 |
| 1647 | RBD | BG2NUC-LC205 |
| 1648 | 2P | BG2NUC-LC206 |
| 1649 | RBD | BG2NUC-LC207 |
| 1650 | 2P | BG2NUC-LC208 |
| 1651 | 2P | BGZNUC-LC209 |
| 1652 | 2P | BG2NUC-LC210 |
| 1653 | RBD | BG2NUC-LC211 |
| 1654 | 2P | BG2NUC-LC212 |
| 1655 | RBD | BG2NUC-LC213 |
| 1656 | 2P | BG2NUC-LC214 |
| 1657 | RBD | BG2NUC-LC215 |
| 1658 | 2P | BG2NUC-LC216 |
| 1659 | RBD | BG2NUC-LC217 |
| 1660 | RBD | BG2NUC-LC218 |
| 1661 | RBD | BG2NUC-LC219 |
| 1662 | 2P | BG2NUC-LC220 |
| 1663 | RBD | BG2NUC-LC221 |
| 1664 | RBD | BG2NUC-LC222 |
| 1665 | RBD | BG2NUC-LC223 |
| 1666 | 2P | BG2NUC-LC224 |
| 1667 | RBD | BG2NUC-LC225 |
| 1668 | 2P | BG2NUC-LC226 |
| 1669 | 2P | BG2NUC-LC227 |
| 1670 | 2P | BG2NUC-LC228 |
| 1671 | RBD | BG2NUC-LC229 |
| 1672 | 2P | BG2NUC-LC230 |
| 1673 | RBD | BG2NUC-LC231 |
| 1674 | 2P | BG2NUC-LC232 |
| 1675 | 2P | BG2NUC-LC233 |
| 1676 | 2P | BG2NUC-LC234 |
| 1677 | RBD | BG2NUC-LC235 |
| 1678 | RBD | BG2NUC-LC236 |
| 1679 | 2P | BG2NUC-LC237 |
| 1680 | RBD | BG2NUC-LC238 |
| 1681 | 2P | BG2NUC-LC239 |
| 1682 | 2P | BG2NUC-LC240 |
| 1683 | 2P | BG2NUC-LC241 |
| 1684 | RBD | BG2NUC-LC242 |
| 1685 | 2P | BG2NUC-LC243 |
| 1686 | RBD | BG2NUC-LC244 |
| 1687 | RBD | BG2NUC-LC245 |
| 1688 | 2P | BG2NUC-LC246 |
| 1689 | RBD | BG2NUC-LC247 |
| 1690 | RBD | BG2NUC-LC248 |
| 1691 | 2P | BG2NUC-LC249 |
| 1692 | 2P | BG2NUC-LC250 |
| 1693 | 2P | BG2NUC-LC251 |
| 1694 | RBD | BG2NUC-LC252 |
| 1695 | 2P | BG2NUC-LC253 |
| 1696 | RBD | BG2NUC-LC254 |
| 1697 | 2P | BG2NUC-LC255 |
| 1698 | RBD | BG2NUC-LC256 |
| 1699 | 2P | BG2NUC-LC257 |
| 1700 | 2P | BG2NUC-LC258 |
| 1701 | 2P | BG2NUC-LC259 |
| 1702 | 2P | BG2NUC-LC260 |
| 1703 | 2P | BG2NUC-LC261 |
| 1704 | RBD | BG2NUC-LC262 |
| 1705 | 2P | BG2NUC-LC263 |
| 1706 | RBD | BG2NUC-LC264 |
| 1707 | 2P | BG2NUC-LC265 |
| 1708 | 2P | BG2NUC-LC266 |
| 1709 | 2P | BG2NUC-LC267 |
| 1710 | 2P | BG2NUC-LC268 |
| 1711 | RBD | BG2NUC-LC269 |
| 1712 | 2P | BG2NUC-LC270 |
| 1713 | 2P | BG2NUC-LC271 |
| 1714 | 2P | BG2NUC-LC272 |
| 1715 | 2P | BG2NUC-LC273 |
| 1716 | 2P | BG2NUC-LC274 |
| 1717 | 2P | BG2NUC-LC275 |
| 1718 | 2P | BG2NUC-LC276 |
| 1719 | 2P | BG2NUC-LC277 |
| 1720 | 2P | BG2NUC-LC278 |
| 1721 | 2P | BG2NUC-LC279 |
| 1722 | RBD | BG2NUC-LC280 |
| 1723 | RBD | BG2NUC-LC281 |
| 1724 | RBD | BG2NUC-LC282 |
| 1725 | 2P | BG2NUC-LC283 |
| 1726 | 2P | BG2NUC-LC284 |
| 1727 | 2P | BG2NUC-LC285 |
| 1728 | 2P | BG2NUC-LC286 |
| 1729 | RBD | BG2NUC-LC287 |
| 1730 | 2P | BG2NUC-LC288 |
| 1731 | 2P | BG2NUC-LC289 |
| 1732 | RBD | BG2NUC-LC290 |
| 1733 | 2P | BG2NUC-LC291 |
| 1734 | 2P | BG2NUC-LC292 |
| 1735 | 2P | BG2NUC-LC293 |
| 1736 | 2P | BG2NUC-LC294 |
| 1737 | RBD | BG2NUC-LC295 |
| 1738 | 2P | BG2NUC-LC296 |
| 1739 | 2F | BG2NUC-LC297 |
| 1740 | RBD | BG2NUC-LC298 |
| 1741 | 2P | BG2NUC-LC299 |
| 1742 | 2P | BG2NUC-LC300 |
| 1743 | 2P | BG2NUC-LC301 |
| 1744 | 2P | BG2NUC-LC302 |
| 1745 | 2P | BG2NUC-LC303 |
| 1746 | 2P | BG2NUC-LC304 |
| 1747 | RBD | BG2NUC-LC305 |
| 1748 | 2P | BG2NUC-LC306 |
| 1749 | 2P | BG2NUC-LC307 |
| 1750 | 2P | BG2NUC-LC308 |
| 1751 | 2P | BG2NUC-LC309 |
| 1752 | RBD | BG2NUC-LC310 |
| 1753 | RBD | BG2NUC-LC311 |
| 1754 | RBD | BG2NUC-LC312 |
| 1755 | RBD | BG2NUC-LC313 |
| 1756 | RBD | BG2NUC-LC314 |
| 1757 | RBD | BG2NUC-LC315 |
| 1758 | 2P | BG2NUC-LC316 |
| 1759 | RBD | BG2NUC-LC317 |
| 1760 | 2P | BG2NUC-LC318 |
| 1761 | 2P | BG2NUC-LC319 |
| 1762 | 2P | BG2NUC-LC320 |
| 1763 | 2P | BG2NUC-LC321 |
| 1764 | RBD | BG2NUC-LC322 |
| 1765 | RBD | BG2NUC-LC323 |
| 1766 | RBD | BG2NUC-LC324 |
| 1767 | RBD | BG2NUC-LC325 |
| 1768 | RBD | BG2NUC-LC326 |
| 1769 | 2P | BG2NUC-LC327 |
| 1770 | 2P | BG2NUC-LC328 |
| 1771 | 2P | BG2NUC-LC329 |
| 1772 | 2P | BG2NUC-LC330 |
| 1773 | 2P | BG2NUC-LC331 |
| 1774 | 2P | BG2NUC-LC332 |
| 1775 | 2P | BG2NUC-LC333 |
| 1776 | RBD | BG2NUC-LC334 |
| 1777 | 2P | BG2NUC-LC335 |
| 1778 | 2P | BG2NUC-LC336 |
| 1779 | RBD | BG2NUC-LC337 |
| 1780 | 2P | BG2NUC-LC338 |
| 1781 | 2P | BG2NUC-LC339 |
| 1782 | 2P | BG2NUC-LC340 |
| 1783 | 2P | BG2NUC-LC341 |
| 1784 | 2P | BG2NUC-LC342 |
| 1785 | 2P | BG2NUC-LC343 |
| 1786 | 2P | BG2NUC-LC344 |
| 1787 | RBD | BG2NUC-LC345 |
| 1788 | 2P | BG2NUC-LC346 |
| 1789 | 2P | BG2NUC-LC347 |
| 1790 | 2P | BG2NUC-LC348 |
| 1791 | RBD | BG2NUC-LC349 |
| 1792 | RBD | BG2NUC-LC350 |
| 1793 | 2P | BG2NUC-LC351 |
| 1794 | RBD | BG2NUC-LC352 |
| 1795 | RBD | BG2NUC-LC353 |
| 1796 | 2P | BG2NUC-LC354 |
| 1797 | 2P | BG2NUC-LC355 |
| 1798 | 2P | BG2NUC-LC356 |
| 1799 | 2P | BG2NUC-LC357 |
| 1800 | 2P | BG2NUC-LC358 |
| 1801 | 2P | BG2NUC-LC359 |
| 1802 | 2P | BG2NUC-LC360 |
| 1803 | 2P | BG2NUC-LC361 |
| 1804 | 2P | BG2NUC-LC362 |
| 1805 | 2P | BG2NUC-LC363 |
| 1806 | RBD | BG2NUC-LC364 |
| 1807 | 2P | BG2NUC-LC365 |
| 1808 | 2P | BG2NUC-LC366 |
| 1809 | 2P | BG2NUC-LC367 |
| 1810 | RBD | BG2NUC-LC368 |
| 1811 | 2P | BG2NUC-LC369 |
| 1812 | 2P | BG2NUC-LC370 |
| 1813 | 2P | BG2NUC-LC371 |
| 1814 | 2P | BG2NUC-LC372 |
| 1815 | 2P | BG2NUC-LC373 |
| 1816 | 2P | BG2NUC-LC374 |
| 1817 | 2P | BG2NUC-LC375 |
| 1818 | 2P | BG2NUC-LC376 |
| 1819 | 2P | BG2NUC-LC377 |
| 1820 | 2P | BG2NUC-LC378 |
| 1821 | 2P | BG2NUC-LC379 |
| 1822 | 2P | BG2NUC-LC380 |
| 1823 | 2P | BG4NUC-HC1 |
| 1824 | 2P | BG4NUC-HC2 |
| 1825 | 2P | BG4NUC-HC3 |
| 1826 | 2P | BG4NUC-HC4 |
| 1827 | 2P | BG4NUC-HC5 |
| 1828 | 2P | BG4NUC-HC6 |
| 1829 | 2P | BG4NUC-HC7 |
| 1830 | 2P | BG4NUC-HC8 |
| 1831 | 2P | BG4NUC-HC9 |
| 1832 | 2P | BG4NUC-HC10 |
| 1833 | 2P | BG4NUC-HC11 |
| 1834 | 2P | BG4NUC-HC12 |
| 1835 | 2P | BG4NUC-HC13 |
| 1836 | 2P | BG4NUC-HC14 |
| 1837 | RBD | BG4NUC-HC15 |
| 1838 | 2P | BG4NUC-HC16 |
| 1839 | 2P | BG4NUC-HC17 |
| 1840 | 2P | BG4NUC-HC18 |
| 1841 | RBD | BG4NUC-HC19 |
| 1842 | 2P | BG4NUC-HC20 |
| 1843 | 2P | BG4NUC-HC21 |
| 1844 | RBD | BG4NUC-HC22 |
| 1845 | RBD | BG4NUC-HC23 |
| 1846 | 2P | BG4NUC-HC24 |
| 1847 | RBD | BG4NUC-HC25 |
| 1848 | RBD | BG4NUC-HC26 |
| 1849 | RBD | BG4NUC-HC27 |
| 1850 | RBD | BG4NUC-HC28 |
| 1851 | RBD | BG4NUC-HC29 |
| 1852 | RBD | BG4NUC-HC30 |
| 1853 | RBD | BG4NUC-HC31 |
| 1854 | RBD | BG4NUC-HC32 |
| 1855 | RBD | BG4NUC-HC33 |
| 1856 | RBD | BG4NUC-HC34 |
| 1857 | 2P | BG4NUC-HC35 |
| 1858 | RBD | BG4NUC-HC36 |
| 1859 | RBD | BG4NUC-HC37 |
| 1860 | RBD | BG4NUC-HC38 |
| 1861 | 2P | BG4NUC-HC39 |
| 1862 | 2P | BG4NUC-HC40 |
| 1863 | 2P | BG4NUC-HC41 |
| 1864 | RBD | BG4NUC-HC42 |
| 1865 | 2P | BG4NUC-HC43 |
| 1866 | 2P | BG4NUC-HC44 |
| 1867 | RBD | BG4NUC-HC45 |
| 1868 | 2P | BG4NUC-HC46 |
| 1869 | 2P | BG4NUC-HC47 |
| 1870 | RBD | BG4NUC-HC48 |
| 1871 | RBD | BG4NUC-HC49 |
| 1872 | 2P | BG4NUC-HC50 |
| 1873 | RBD | BG4NUC-HC51 |
| 1874 | 2P | BG4NUC-HC52 |
| 1875 | 2P | BG4NUC-HC53 |
| 1876 | RBD | BG4NUC-HC54 |
| 1877 | 2P | BG4NUC-HC55 |
| 1878 | 2P | BG4NUC-HC56 |
| 1879 | 2P | BG4NUC-HC57 |
| 1880 | RBD | BG4NUC-HC58 |
| 1881 | RBD | BG4NUC-HC59 |
| 1882 | 2P | BG4NUC-HC60 |
| 1883 | 2P | BG4NUC-HC61 |
| 1884 | 2P | BG4NUC-HC62 |
| 1885 | RBD | BG4NUC-HC63 |
| 1886 | RBD | BG4NUC-HC64 |
| 1887 | RBD | BG4NUC-HC65 |
| 1888 | 2P | BG4NUC-HC66 |
| 1889 | 2P | BG4NUC-HC67 |
| 1890 | 2P | BG4NUC-HC68 |
| 1891 | RBD | BG4NUC-HC69 |
| 1892 | 2P | BG4NUC-HC70 |
| 1893 | RBD | BG4NUC-HC71 |
| 1894 | 2P | BG4NUC-HC72 |
| 1895 | RBD | BG4NUC-HC73 |
| 1896 | 2P | BG4NUC-HC74 |
| 1897 | RBD | BG4NUC-HC75 |
| 1898 | 2P | BG4NUC-HC76 |
| 1899 | 2P | BG4NUC-HC77 |
| 1900 | 2P | BG4NUC-HC78 |
| 1901 | 2P | BG4NUC-HC79 |
| 1902 | RBD | BG4NUC-HC80 |
| 1903 | RBD | BG4NUC-HC81 |
| 1904 | RBD | BG4NUC-HC82 |
| 1905 | RBD | BG4NUC-HC83 |
| 1906 | 2P | BG4NUC-HC84 |
| 1907 | 2P | BG4NUC-HC85 |
| 1908 | 2P | BG4NUC-HC86 |
| 1909 | 2P | BG4NUC-HC87 |
| 1910 | 2P | BG4NUC-HC88 |
| 1911 | RBD | BG4NUC-HC89 |
| 1912 | RBD | BG4NUC-HC90 |
| 1913 | 2P | BG4NUC-HC91 |
| 1914 | 2P | BG4NUC-HC92 |
| 1915 | 2P | BG4NUC-HC93 |
| 1916 | RBD | BG4NUC-HC94 |
| 1917 | 2P | BG4NUC-HC95 |
| 1918 | RBD | BG4NUC-HC96 |
| 1919 | RBD | BG4NUC-HC97 |
| 1920 | RBD | BG4NUC-HC98 |
| 1921 | 2P | BG4NUC-HC99 |
| 1922 | 2P | BG4NUC-HC100 |
| 1923 | 2P | BG4NUC-HC101 |
| 1924 | RBD | BG4NUC-HC102 |
| 1925 | 2P | BG4NUC-HC103 |
| 1926 | 2P | BG4NUC-HC104 |
| 1927 | 2P | BG4NUC-HC105 |
| 1928 | 2P | BG4NUC-HC106 |
| 1929 | 2P | BG4NUC-HC107 |
| 1930 | 2P | BG4NUC-HC108 |
| 1931 | 2P | BG4NUC-HC109 |
| 1932 | 2P | BG4NUC-HC110 |
| 1933 | 2P | BG4NUC-HC111 |
| 1934 | 2P | BG4NUC-HC112 |
| 1935 | RBD | BG4NUC-HC113 |
| 1936 | RBD | BG4NUC-HC114 |
| 1937 | RBD | BG4NUC-HC115 |
| 1938 | 2P | BG4NUC-HC116 |
| 1939 | RBD | BG4NUC-HC117 |
| 1940 | 2P | BG4NUC-HC118 |
| 1941 | 2P | BG4NUC-HC119 |
| 1942 | 2P | BG4NUC-HC120 |
| 1943 | RBD | BG4NUC-HC121 |
| 1944 | 2P | BG4NUC-HC122 |
| 1945 | RBD | BG4NUC-HC123 |
| 1946 | 2P | BG4NUC-HC124 |
| 1947 | RBD | BG4NUC-HC125 |
| 1948 | RBD | BG4NUC-HC126 |
| 1949 | 2P | BG4NUC-HC127 |
| 1950 | RBD | BG4NUC-HC128 |
| 1951 | RBD | BG4NUC-HC129 |
| 1952 | 2P | BG4NUC-HC130 |
| 1953 | 2P | BG4NUC-HC131 |
| 1954 | 2P | BG4NUC-HC132 |
| 1955 | 2P | BG4NUC-HC133 |
| 1956 | RBD | BG4NUC-HC134 |
| 1957 | 2P | BG4NUC-HC135 |
| 1958 | 2P | BG4NUC-HC136 |
| 1959 | 2P | BG4NUC-HC137 |
| 1960 | 2P | BG4NUC-HC138 |
| 1961 | 2P | BG4NUC-HC139 |
| 1962 | 2P | BG4NUC-HC140 |
| 1963 | 2P | BG4NUC-HC141 |
| 1964 | 2P | BG4NUC-HC142 |
| 1965 | 2P | BG4NUC-HC143 |
| 1966 | RBD | BG4NUC-HC144 |
| 1967 | 2P | BG4NUC-HC145 |
| 1968 | 2P | BG4NUC-HC146 |
| 1969 | 2P | BG4NUC-HC147 |
| 1970 | 2P | BG4NUC-HC148 |
| 1971 | 2P | BG4NUC-HC149 |
| 1972 | 2P | BG4NUC-HC150 |
| 1973 | 2P | BG4NUC-HC151 |
| 1974 | 2P | BG4NUC-HC152 |
| 1975 | 2P | BG4NUC-HC153 |
| 1976 | 2P | BG4NUC-HC154 |
| 1977 | 2P | BG4NUC-HC155 |
| 1978 | RBD | BG4NUC-HC156 |
| 1979 | 2P | BG4NUC-HC157 |
| 1980 | RBD | BG4NUC-HC158 |
| 1981 | 2P | BG4NUC-HC159 |
| 1982 | RBD | BG4NUC-HC160 |
| 1983 | RBD | BG4NUC-HC161 |
| 1984 | RBD | BG4NUC-HC162 |
| 1985 | 2P | BG4NUC-HC163 |
| 1986 | RBD | BG4NUC-HC164 |
| 1987 | RBD | BG4NUC-HC165 |
| 1988 | RBD | BG4NUC-HC166 |
| 1989 | RBD | BG4NUC-HC167 |
| 1990 | 2P | BG4NUC-HC168 |
| 1991 | 2P | BG4NUC-HC169 |
| 1992 | 2P | BG4NUC-HC170 |
| 1993 | RBD | BG4NUC-HC171 |
| 1994 | 2P | BG4NUC-HC172 |
| 1995 | 2P | BG4NUC-HC173 |
| 1996 | RBD | BG4NUC-HC174 |
| 1997 | 2P | BG4NUC-HC175 |
| 1998 | RBD | BG4NUC-HC176 |
| 1999 | 2P | BG4NUC-HC177 |
| 2000 | 2P | BG4NUC-HC178 |
| 2001 | 2P | BG4NUC-HC179 |
| 2002 | RBD | BG4NUC-HC180 |
| 2003 | 2P | BG4NUC-HC181 |
| 2004 | RBD | BG4NUC-HC182 |
| 2005 | 2P | BG4NUC-HC183 |
| 2006 | 2P | BG4NUC-HC184 |
| 2007 | 2P | BG4NUC-HC185 |
| 2008 | 2P | BG4NUC-HC186 |
| 2009 | 2P | BG4NUC-HC187 |
| 2010 | 2P | BG4NUC-HC188 |
| 2011 | 2P | BG4NUC-HC189 |
| 2012 | 2P | BG4NUC-HC190 |
| 2013 | 2P | BG4NUC-HC191 |
| 2014 | 2P | BG4NUC-HC192 |
| 2015 | RBD | BG4NUC-HC193 |
| 2016 | 2P | BG4NUC-HC194 |
| 2017 | RBD | BG4NUC-HC195 |
| 2018 | RBD | BG4NUC-HC196 |
| 2019 | RBD | BG4NUC-HC197 |
| 2020 | 2P | BG4NUC-HC198 |
| 2021 | 2P | BG4NUC-HC199 |
| 2022 | 2P | BG4NUC-HC200 |
| 2023 | 2P | BG4NUC-HC201 |
| 2024 | 2P | BG4NUC-HC202 |
| 2025 | 2P | BG4NUC-HC203 |
| 2026 | 2P | BG4NUC-HC204 |
| 2027 | 2P | BG4NUC-HC205 |
| 2028 | 2P | BG4NUC-HC206 |
| 2029 | 2P | BG4NUC-HC207 |
| 2030 | 2P | BG4NUC-HC208 |
| 2031 | 2P | BG4NUC-HC209 |
| 2032 | RBD | BG4NUC-HC210 |
| 2033 | RBD | BG4NUC-HC211 |
| 2034 | RBD | BG4NUC-HC212 |
| 2035 | 2P | BG4NUC-HC213 |
| 2036 | 2P | BG4NUC-HC214 |
| 2037 | 2P | BG4NUC-HC215 |
| 2038 | 2P | BG4NUC-HC216 |
| 2039 | RBD | BG4NUC-HC217 |
| 2040 | 2P | BG4NUC-HC218 |
| 2041 | RBD | BG4NUC-HC219 |
| 2042 | RBD | BG4NUC-HC220 |
| 2043 | 2P | BG4NUC-HC221 |
| 2044 | RBD | BG4NUC-HC222 |
| 2045 | RBD | BG4NUC-HC223 |
| 2046 | 2P | BG4NUC-HC224 |
| 2047 | RBD | BG4NUC-HC225 |
| 2048 | RBD | BG4NUC-HC226 |
| 2049 | 2P | BG4NUC-HC227 |
| 2050 | RBD | BG4NUC-HC228 |
| 2051 | 2P | BG4NUC-HC229 |
| 2052 | 2P | BG4NUC-HC230 |
| 2053 | RBD | BG4NUC-HC231 |
| 2054 | 2P | BG4NUC-HC232 |
| 2055 | 2P | BG4NUC-HC233 |
| 2056 | 2P | BG4NUC-HC234 |
| 2057 | RBD | BG4NUC-HC235 |
| 2058 | RBD | BG4NUC-HC236 |
| 2059 | 2P | BG4NUC-HC237 |
| 2060 | 2P | BG4NUC-HC238 |
| 2061 | 2P | BG4NUC-HC239 |
| 2062 | 2P | BG4NUC-HC240 |
| 2063 | 2P | BG4NUC-HC241 |
| 2064 | 2P | BG4NUC-HC242 |
| 2065 | 2P | BG4NUC-HC243 |
| 2066 | 2P | BG4NUC-HC244 |
| 2067 | 2P | BG4NUC-HC245 |
| 2068 | 2P | BG4NUC-HC246 |
| 2069 | RBD | BG4NUC-HC247 |
| 2070 | RBD | BG4NUC-HC248 |
| 2071 | RBD | BG4NUC-HC249 |
| 2072 | RBD | BG4NUC-HC250 |
| 2073 | 2P | BG4NUC-HC251 |
| 2074 | 2P | BG4NUC-HC252 |
| 2075 | 2F | BG4NUC-HC253 |
| 2076 | RBD | BG4NUC-HC254 |
| 2077 | 2P | BG4NUC-HC255 |
| 2078 | RBD | BG4NUC-HC256 |
| 2079 | RBD | BG4NUC-HC257 |
| 2080 | RBD | BG4NUC-HC258 |
| 2081 | 2P | BG4NUC-HC259 |
| 2082 | RBD | BG4NUC-HC260 |
| 2083 | 2F | BG4NUC-HC261 |
| 2084 | RBD | BG4NUC-HC262 |
| 2085 | RBD | BG4NUC-HC263 |
| 2086 | RBD | BG4NUC-HC264 |
| 2087 | 2F | BG4NUC-HC265 |
| 2088 | RBD | BG4NUC-HC266 |
| 2089 | 2P | BG4NUC-HC267 |
| 2090 | 2P | BG4NUC-HC268 |
| 2091 | RBD | BG4NUC-HC269 |
| 2092 | RBD | BG4NUC-HC270 |
| 2093 | RBD | BG4NUC-HC271 |
| 2094 | 2P | BG4NUC-HC272 |
| 2095 | RBD | BG4NUC-HC273 |
| 2096 | 2P | BG4NUC-HC274 |
| 2097 | 2P | BG4NUC-HC275 |
| 2098 | RBD | BG4NUC-HC276 |
| 2099 | RBD | BG4NUC-HC277 |
| 2100 | RBD | BG4NUC-HC278 |
| 2101 | 2P | BG4NUC-HC279 |
| 2102 | RBD | BG4NUC-HC280 |
| 2103 | RBD | BG4NUC-HC281 |
| 2104 | 2P | BG4NUC-HC282 |
| 2105 | 2P | BG4NUC-HC283 |
| 2106 | 2P | BG4NUC-HC284 |
| 2107 | RBD | BG4NUC-HC285 |
| 2108 | 2P | BG4NUC-HC286 |
| 2109 | 2P | BG4NUC-HC287 |
| 2110 | RBD | BG4NUC-HC288 |
| 2111 | 2P | BG4NUC-HC289 |
| 2112 | 2P | BG4NUC-HC290 |
| 2113 | RBD | BG4NUC-HC291 |
| 2114 | 2P | BG4NUC-HC292 |
| 2115 | RBD | BG4NUC-HC293 |
| 2116 | 2P | BG4NUC-HC294 |
| 2117 | RBD | BG4NUC-HC295 |
| 2118 | 2P | BG4NUC-HC296 |
| 2119 | RBD | BG4NUC-HC297 |
| 2120 | 2P | BG4NUC-HC298 |
| 2121 | 2P | BG4NUC-HC299 |
| 2122 | 2P | BG4NUC-HC300 |
| 2123 | 2P | BG4NUC-HC301 |
| 2124 | 2P | BG4NUC-HC302 |
| 2125 | 2P | BG4NUC-HC303 |
| 2126 | 2F | BG4NUC-HC304 |
| 2127 | 2P | BG4NUC-HC305 |
| 2128 | RBD | BG4NUC-HC306 |
| 2129 | 2P | BG4NUC-HC307 |
| 2130 | 2P | BG4NUC-HC308 |
| 2131 | 2P | BG4NUC-HC309 |
| 2132 | 2P | BG4NUC-HC310 |
| 2133 | RBD | BG4NUC-HC311 |
| 2134 | 2P | BG4NUC-HC312 |
| 2135 | 2F | BG4NUC-HC313 |
| 2136 | 2P | BG4NUC-HC314 |
| 2137 | 2P | BG4NUC-HC315 |
| 2138 | 2F | BG4NUC-HC316 |
| 2139 | RBD | BG4NUC-HC317 |
| 2140 | RBD | BG4NUC-HC318 |
| 2141 | 2P | BG4NUC-HC319 |
| 2142 | 2P | BG4NUC-HC320 |
| 2143 | 2P | BG4NUC-HC321 |
| 2144 | RBD | BG4NUC-HC322 |
| 2145 | 2P | BG4NUC-HC323 |
| 2146 | RBD | BG4NUC-HC324 |
| 2147 | 2P | BG4NUC-HC325 |
| 2148 | RBD | BG4NUC-HC326 |
| 2149 | RBD | BG4NUC-HC327 |
| 2150 | RBD | BG4NUC-HC328 |
| 2151 | 2P | BG4NUC-HC329 |
| 2152 | 2P | BG4NUC-HC330 |
| 2153 | RBD | BG4NUC-HC331 |
| 2154 | 2P | BG4NUC-HC332 |
| 2155 | 2P | BG4NUC-HC333 |
| 2156 | 2P | BG4NUC-HC334 |
| 2157 | 2P | BG4NUC-HC335 |
| 2158 | RBD | BG4NUC-HC336 |
| 2159 | 2P | BG4NUC-HC337 |
| 2160 | RBD | BG4NUC-HC338 |
| 2161 | 2P | BG4NUC-HC339 |
| 2162 | RBD | BG4NUC-HC340 |
| 2163 | 2P | BG4NUC-HC341 |
| 2164 | RBD | BG4NUC-HC342 |
| 2165 | RBD | BG4NUC-HC343 |
| 2166 | RBD | BG4NUC-HC344 |
| 2167 | 2P | BG4NUC-HC345 |
| 2168 | RBD | BG4NUC-HC346 |
| 2169 | 2P | BG4NUC-HC347 |
| 2170 | RBD | BG4NUC-HC348 |
| 2171 | 2P | BG4NUC-HC349 |
| 2172 | 2P | BG4NUC-HC350 |
| 2173 | 2P | BG4NUC-HC351 |
| 2174 | 2P | BG4NUC-HC352 |
| 2175 | RBD | BG4NUC-HC353 |
| 2176 | RBD | BG4NUC-HC354 |
| 2177 | RBD | BG4NUC-HC355 |
| 2178 | 2P | BG4NUC-HC356 |
| 2179 | 2P | BG4NUC-HC357 |
| 2180 | RBD | BG4NUC-HC358 |
| 2181 | RBD | BG4NUC-HC359 |
| 2182 | RBD | BG4NUC-HC360 |
| 2183 | RBD | BG4NUC-HC361 |
| 2184 | RBD | BG4NUC-HC362 |
| 2185 | 2P | BG4NUC-HC363 |
| 2186 | 2P | BG4NUC-HC364 |
| 2187 | RBD | BG4NUC-HC365 |
| 2188 | 2P | BG4NUC-HC366 |
| 2189 | 2P | BG4NUC-HC367 |
| 2190 | 2P | BG4NUC-HC368 |
| 2191 | 2P | BG4NUC-HC369 |
| 2192 | 2P | BG4NUC-HC370 |
| 2193 | 2P | BG4NUC-HC371 |
| 2194 | 2P | BG4NUC-HC372 |
| 2195 | 2P | BG4NUC-HC373 |
| 2196 | 2P | BG4NUC-HC374 |
| 2197 | RBD | BG4NUC-HC375 |
| 2198 | 2P | BG4NUC-HC376 |
| 2199 | 2P | BG4NUC-HC377 |
| 2200 | 2P | BG4NUC-HC378 |
| 2201 | RBD | BG4NUC-HC379 |
| 2202 | 2P | BG4NUC-HC380 |
| 2203 | 2P | BG4NUC-HC381 |
| 2204 | 2P | BG4NUC-HC382 |
| 2205 | 2P | BG4NUC-HC383 |
| 2206 | 2P | BG4NUC-HC384 |
| 2207 | 2P | BG4NUC-HC385 |
| 2208 | RBD | BG4NUC-HC386 |
| 2209 | RBD | BG4NUC-HC387 |
| 2210 | 2P | BG4NUC-HC388 |
| 2211 | 2P | BG4NUC-HC389 |
| 2212 | 2P | BG4NUC-HC390 |
| 2213 | 2P | BG4NUC-HC391 |
| 2214 | 2P | BG4NUC-HC392 |
| 2215 | 2P | BG4NUC-HC393 |
| 2216 | RBD | BG4NUC-HC394 |
| 2217 | 2P | BG4NUC-HC395 |
| 2218 | 2P | BG4NUC-HC396 |
| 2219 | 2P | BG4NUC-HC397 |
| 2220 | 2P | BG4NUC-HC398 |
| 2221 | 2P | BG4NUC-HC399 |
| 2222 | RBD | BG4NUC-HC400 |
| 2223 | RBD | BG4NUC-HC401 |
| 2224 | 2P | BG4NUC-HC402 |
| 2225 | 2P | BG4NUC-HC403 |
| 2226 | 2F | BG4NUC-HC404 |
| 2227 | RBD | BG4NUC-HC405 |
| 2228 | 2P | BG4NUC-HC406 |
| 2229 | RBD | BG4NUC-HC407 |
| 2230 | 2P | BG4NUC-HC408 |
| 2231 | 2P | BG4NUC-HC409 |
| 2232 | 2P | BG4NUC-LC1 |
| 2233 | 2P | BG4NUC-LC2 |
| 2234 | 2P | BG4NUC-LC3 |
| 2235 | 2P | BG4NUC-LC4 |
| 2236 | 2P | BG4NUC-LC5 |
| 2237 | 2P | BG4NUC-LC6 |
| 2238 | 2P | BG4NUC-LC7 |
| 2239 | 2F | BG4NUC-LC8 |
| 2240 | 2F | BG4NUC-LC9 |
| 2241 | 2P | BG4NUC-LC10 |
| 2242 | 2P | BG4NUC-LC11 |
| 2243 | 2P | BG4NUC-LC12 |
| 2244 | 2F | BG4NUC-LC13 |
| 2245 | 2P | BG4NUC-LC14 |
| 2246 | RBD | BG4NUC-LC15 |
| 2247 | 2P | BG4NUC-LC16 |
| 2248 | 2P | BG4NUC-LC17 |
| 2249 | 2P | BG4NUC-LC18 |
| 2250 | RBD | BG4NUC-LC19 |
| 2251 | 2P | BG4NUC-LC20 |
| 2252 | 2P | BG4NUC-LC21 |
| 2253 | RBD | BG4NUC-LC22 |
| 2254 | RBD | BG4NUC-LC23 |
| 2255 | 2P | BG4NUC-LC24 |
| 2256 | RBD | BG4NUC-LC25 |
| 2257 | RBD | BG4NUC-LC26 |
| 2258 | RBD | BG4NUC-LC27 |
| 2259 | RBD | BG4NUC-LC28 |
| 2260 | RBD | BG4NUC-LC29 |
| 2261 | RBD | BG4NUC-LC30 |
| 2262 | RBD | BG4NUC-LC31 |
| 2263 | RBD | BG4NUC-LC32 |
| 2264 | RBD | BG4NUC-LC33 |
| 2265 | RBD | BG4NUC-LC34 |
| 2266 | 2P | BG4NUC-LC35 |
| 2267 | RBD | BG4NUC-LC36 |
| 2268 | RBD | BG4NUC-LC37 |
| 2269 | RBD | BG4NUC-LC38 |
| 2270 | 2P | BG4NUC-LC39 |
| 2271 | 2P | BG4NUC-LC40 |
| 2272 | 2P | BG4NUC-LC41 |
| 2273 | RBD | BG4NUC-LC42 |
| 2274 | 2P | BG4NUC-LC43 |
| 2275 | 2P | BG4NUC-LC44 |
| 2276 | RBD | BG4NUC-LC45 |
| 2277 | 2P | BG4NUC-LC46 |
| 2278 | 2P | BG4NUC-LC47 |
| 2279 | RBD | BG4NUC-LC48 |
| 2280 | RBD | BG4NUC-LC49 |
| 2281 | 2P | BG4NUC-LC50 |
| 2282 | RBD | BG4NUC-LC51 |
| 2283 | 2P | BG4NUC-LC52 |
| 2284 | 2P | BG4NUC-LC53 |
| 2285 | RBD | BG4NUC-LC54 |
| 2286 | 2P | BG4NUC-LC55 |
| 2287 | 2P | BG4NUC-LC56 |
| 2288 | 2P | BG4NUC-LC57 |
| 2289 | RBD | BG4NUC-LC58 |
| 2290 | RBD | BG4NUC-LC59 |
| 2291 | 2P | BG4NUC-LC60 |
| 2292 | 2P | BG4NUC-LC61 |
| 2293 | 2P | BG4NUC-LC62 |
| 2294 | RBD | BG4NUC-LC63 |
| 2295 | RBD | BG4NUC-LC64 |
| 2296 | RBD | BG4NUC-LC65 |
| 2297 | 2P | BG4NUC-LC66 |
| 2298 | 2P | BG4NUC-LC67 |
| 2299 | 2P | BG4NUC-LC68 |
| 2300 | RBD | BG4NUC-LC69 |
| 2301 | 2P | BG4NUC-LC70 |
| 2302 | RBD | BG4NUC-LC71 |
| 2303 | 2P | BG4NUC-LC72 |
| 2304 | RBD | BG4NUC-LC73 |
| 2305 | 2P | BG4NUC-LC74 |
| 2306 | RBD | BG4NUC-LC75 |
| 2307 | 2P | BG4NUC-LC76 |
| 2308 | 2P | BG4NUC-LC77 |
| 2309 | 2P | BG4NUC-LC78 |
| 2310 | 2P | BG4NUC-LC79 |
| 2311 | RBD | BG4NUC-LC80 |
| 2312 | RBD | BG4NUC-LC81 |
| 2313 | RBD | BG4NUC-LC82 |
| 2314 | RBD | BG4NUC-LC83 |
| 2315 | 2P | BG4NUC-LC84 |
| 2316 | 2P | BG4NUC-LC85 |
| 2317 | 2P | BG4NUC-LC86 |
| 2318 | 2P | BG4NUC-LC87 |
| 2319 | 2P | BG4NUC-LC88 |
| 2320 | RBD | BG4NUC-LC89 |
| 2321 | RBD | BG4NUC-LC90 |
| 2322 | 2P | BG4NUC-LC91 |
| 2323 | 2P | BG4NUC-LC92 |
| 2324 | 2P | BG4NUC-LC93 |
| 2325 | RBD | BG4NUC-LC94 |
| 2326 | 2P | BG4NUC-LC95 |
| 2327 | RBD | BG4NUC-LC96 |
| 2328 | RBD | BG4NUC-LC97 |
| 2329 | RBD | BG4NUC-LC98 |
| 2330 | 2P | BG4NUC-LC99 |
| 2331 | 2P | BG4NUC-LC100 |
| 2332 | 2P | BG4NUC-LC101 |
| 2333 | RBD | BG4NUC-LC102 |
| 2334 | 2P | BG4NUC-LC103 |
| 2335 | 2P | BG4NUC-LC104 |
| 2336 | 2P | BG4NUC-LC105 |
| 2337 | 2P | BG4NUC-LC106 |
| 2338 | 2P | BG4NUC-LC107 |
| 2339 | 2P | BG4NUC-LC108 |
| 2340 | 2P | BG4NUC-LC109 |
| 2341 | 2P | BG4NUC-LC110 |
| 2342 | 2P | BG4NUC-LC111 |
| 2343 | 2P | BG4NUC-LC112 |
| 2344 | RBD | BG4NUC-LC113 |
| 2345 | RBD | BG4NUC-LC114 |
| 2346 | RBD | BG4NUC-LC115 |
| 2347 | 2P | BG4NUC-LC116 |
| 2348 | RBD | BG4NUC-LC117 |
| 2349 | 2P | BG4NUC-LC118 |
| 2350 | 2P | BG4NUC-LC119 |
| 2351 | 2P | BG4NUC-LC120 |
| 2352 | RBD | BG4NUC-LC121 |
| 2353 | 2P | BG4NUC-LC122 |
| 2354 | RBD | BG4NUC-LC123 |
| 2355 | 2P | BG4NUC-LC124 |
| 2356 | RBD | BG4NUC-LC125 |
| 2357 | RBD | BG4NUC-LC126 |
| 2358 | 2P | BG4NUC-LC127 |
| 2359 | RBD | BG4NUC-LC128 |
| 2360 | RBD | BG4NUC-LC129 |
| 2361 | 2P | BG4NUC-LC130 |
| 2362 | 2P | BG4NUC-LC131 |
| 2363 | 2P | BG4NUC-LC132 |
| 2364 | 2P | BG4NUC-LC133 |
| 2365 | RBD | BG4NUC-LC134 |
| 2366 | 2P | BG4NUC-LC135 |
| 2367 | 2P | BG4NUC-LC136 |
| 2368 | 2P | BG4NUC-LC137 |
| 2369 | 2P | BG4NUC-LC138 |
| 2370 | 2P | BG4NUC-LC139 |
| 2371 | 2P | BG4NUC-LC140 |
| 2372 | 2P | BG4NUC-LC141 |
| 2373 | 2P | BG4NUC-LC142 |
| 2374 | 2P | BG4NUC-LC143 |
| 2375 | RBD | BG4NUC-LC144 |
| 2376 | 2P | BG4NUC-LC145 |
| 2377 | 2P | BG4NUC-LC146 |
| 2378 | 2P | BG4NUC-LC147 |
| 2379 | 2P | BG4NUC-LC148 |
| 2380 | 2P | BG4NUC-LC149 |
| 2381 | 2P | BG4NUC-LC150 |
| 2382 | 2P | BG4NUC-LC151 |
| 2383 | 2P | BG4NUC-LC152 |
| 2384 | 2P | BG4NUC-LC153 |
| 2385 | 2P | BG4NUC-LC154 |
| 2386 | 2P | BG4NUC-LC155 |
| 2387 | RBD | BG4NUC-LC156 |
| 2388 | 2P | BG4NUC-LC157 |
| 2389 | RBD | BG4NUC-LC158 |
| 2390 | 2P | BG4NUC-LC159 |
| 2391 | RBD | BG4NUC-LC160 |
| 2392 | RBD | BG4NUC-LC161 |
| 2393 | RBD | BG4NUC-LC162 |
| 2394 | 2P | BG4NUC-LC163 |
| 2395 | RBD | BG4NUC-LC164 |
| 2396 | RBD | BG4NUC-LC165 |
| 2397 | RBD | BG4NUC-LC166 |
| 2398 | RBD | BG4NUC-LC167 |
| 2399 | 2P | BG4NUC-LC168 |
| 2400 | 2P | BG4NUC-LC169 |
| 2401 | 2P | BG4NUC-LC170 |
| 2402 | RBD | BG4NUC-LC171 |
| 2403 | 2P | BG4NUC-LC172 |
| 2404 | 2P | BG4NUC-LC173 |
| 2405 | RBD | BG4NUC-LC174 |
| 2406 | 2P | BG4NUC-LC175 |
| 2407 | RBD | BG4NUC-LC176 |
| 2408 | 2P | BG4NUC-LC177 |
| 2409 | 2P | BG4NUC-LC178 |
| 2410 | 2P | BG4NUC-LC179 |
| 2411 | RBD | BG4NUC-LC180 |
| 2412 | 2P | BG4NUC-LC181 |
| 2413 | RBD | BG4NUC-LC182 |
| 2414 | 2P | BG4NUC-LC183 |
| 2415 | 2P | BG4NUC-LC184 |
| 2416 | 2P | BG4NUC-LC185 |
| 2417 | 2P | BG4NUC-LC186 |
| 2418 | 2P | BG4NUC-LC187 |
| 2419 | 2P | BG4NUC-LC188 |
| 2420 | 2P | BG4NUC-LC189 |
| 2421 | 2P | BG4NUC-LC190 |
| 2422 | 2P | BG4NUC-LC191 |
| 2423 | 2P | BG4NUC-LC192 |
| 2424 | RBD | BG4NUC-LC193 |
| 2425 | 2P | BG4NUC-LC194 |
| 2426 | RBD | BG4NUC-LC195 |
| 2427 | RBD | BG4NUC-LC196 |
| 2428 | RBD | BG4NUC-LC197 |
| 2429 | 2P | BG4NUC-LC198 |
| 2430 | 2P | BG4NUC-LC199 |
| 2431 | 2P | BG4NUC-LC200 |
| 2432 | 2P | BG4NUC-LC201 |
| 2433 | 2P | BG4NUC-LC202 |
| 2434 | 2P | BG4NUC-LC203 |
| 2435 | 2P | BG4NUC-LC204 |
| 2436 | 2P | BG4NUC-LC205 |
| 2437 | 2P | BG4NUC-LC206 |
| 2438 | 2P | BG4NUC-LC207 |
| 2439 | 2P | BG4NUC-LC208 |
| 2440 | 2P | BG4NUC-LC209 |
| 2441 | RBD | BG4NUC-LC210 |
| 2442 | RBD | BG4NUC-LC211 |
| 2443 | RBD | BG4NUC-LC212 |
| 2444 | 2P | BG4NUC-LC213 |
| 2445 | 2P | BG4NUC-LC214 |
| 2446 | 2P | BG4NUC-LC215 |
| 2447 | 2P | BG4NUC-LC216 |
| 2448 | RBD | BG4NUC-LC217 |
| 2449 | 2P | BG4NUC-LC218 |
| 2450 | RBD | BG4NUC-LC219 |
| 2451 | RBD | BG4NUC-LC220 |
| 2452 | 2P | BG4NUC-LC221 |
| 2453 | RBD | BG4NUC-LC222 |
| 2454 | RBD | BG4NUC-LC223 |
| 2455 | 2P | BG4NUC-LC224 |
| 2456 | RBD | BG4NUC-LC225 |
| 2457 | RBD | BG4NUC-LC226 |
| 2458 | 2P | BG4NUC-LC227 |
| 2459 | RBD | BG4NUC-LC228 |
| 2460 | 2P | BG4NUC-LC229 |
| 2461 | 2P | BG4NUC-LC230 |
| 2462 | RBD | BG4NUC-LC231 |
| 2463 | 2P | BG4NUC-LC232 |
| 2464 | 2P | BG4NUC-LC233 |
| 2465 | 2P | BG4NUC-LC234 |
| 2466 | RBD | BG4NUC-LC235 |
| 2467 | RBD | BG4NUC-LC236 |
| 2468 | 2P | BG4NUC-LC237 |
| 2469 | 2P | BG4NUC-LC238 |
| 2470 | 2P | BG4NUC-LC239 |
| 2471 | 2P | BG4NUC-LC240 |
| 2472 | 2P | BG4NUC-LC241 |
| 2473 | 2P | BG4NUC-LC242 |
| 2474 | 2P | BG4NUC-LC243 |
| 2475 | 2P | BG4NUC-LC244 |
| 2476 | 2P | BG4NUC-LC245 |
| 2477 | 2P | BG4NUC-LC246 |
| 2478 | RBD | BG4NUC-LC247 |
| 2479 | RBD | BG4NUC-LC248 |
| 2480 | RBD | BG4NUC-LC249 |
| 2481 | RBD | BG4NUC-LC250 |
| 2482 | 2P | BG4NUC-LC251 |
| 2483 | 2P | BG4NUC-LC252 |
| 2484 | 2P | BG4NUC-LC253 |
| 2485 | RBD | BG4NUC-LC254 |
| 2486 | 2P | BG4NUC-LC255 |
| 2487 | RBD | BG4NUC-LC256 |
| 2488 | RBD | BG4NUC-LC257 |
| 2489 | RBD | BG4NUC-LC258 |
| 2490 | 2P | BG4NUC-LC259 |
| 2491 | RBD | BG4NUC-LC260 |
| 2492 | 2P | BG4NUC-LC261 |
| 2493 | RBD | BG4NUC-LC262 |
| 2494 | RBD | BG4NUC-LC263 |
| 2495 | RBD | BG4NUC-LC264 |
| 2496 | 2P | BG4NUC-LC265 |
| 2497 | RBD | BG4NUC-LC266 |
| 2498 | 2P | BG4NUC-LC267 |
| 2499 | 2P | BG4NUC-LC268 |
| 2500 | RBD | BG4NUC-LC269 |
| 2501 | RBD | BG4NUC-LC270 |
| 2502 | RBD | BG4NUC-LC271 |
| 2503 | 2P | BG4NUC-LC272 |
| 2504 | RBD | BG4NUC-LC273 |
| 2505 | 2P | BG4NUC-LC274 |
| 2506 | 2P | BG4NUC-LC275 |
| 2507 | RBD | BG4NUC-LC276 |
| 2508 | RBD | BG4NUC-LC277 |
| 2509 | RBD | BG4NUC-LC278 |
| 2510 | 2P | BG4NUC-LC279 |
| 2511 | RBD | BG4NUC-LC280 |
| 2512 | RBD | BG4NUC-LC281 |
| 2513 | 2P | BG4NUC-LC282 |
| 2514 | 2P | BG4NUC-LC283 |
| 2515 | 2P | BG4NUC-LC284 |
| 2516 | RBD | BG4NUC-LC285 |
| 2517 | 2P | BG4NUC-LC286 |
| 2518 | 2P | BG4NUC-LC287 |
| 2519 | RBD | BG4NUC-LC288 |
| 2520 | 2P | BG4NUC-LC289 |
| 2521 | 2P | BG4NUC-LC290 |
| 2522 | RBD | BG4NUC-LC291 |
| 2523 | 2F | BG4NUC-LC292 |
| 2524 | RBD | BG4NUC-LC293 |
| 2525 | 2P | BG4NUC-LC294 |
| 2526 | RBD | BG4NUC-LC295 |
| 2527 | 2P | BG4NUC-LC296 |
| 2528 | RBD | BG4NUC-LC297 |
| 2529 | 2P | BG4NUC-LC298 |
| 2530 | 2P | BG4NUC-LC299 |
| 2531 | 2P | BG4NUC-LC300 |
| 2532 | 2P | BG4NUC-LC301 |
| 2533 | 2P | BG4NUC-LC302 |
| 2534 | 2P | BG4NUC-LC303 |
| 2535 | 2P | BG4NUC-LC304 |
| 2536 | 2P | BG4NUC-LC305 |
| 2537 | RBD | BG4NUC-LC306 |
| 2538 | 2P | BG4NUC-LC307 |
| 2539 | 2P | BG4NUC-LC308 |
| 2540 | 2P | BG4NUC-LC309 |
| 2541 | 2P | BG4NUC-LC310 |
| 2542 | RBD | BG4NUC-LC311 |
| 2543 | 2P | BG4NUC-LC312 |
| 2544 | 2P | BG4NUC-LC313 |
| 2545 | 2P | BG4NUC-LC314 |
| 2546 | 2P | BG4NUC-LC315 |
| 2547 | 2P | BG4NUC-LC316 |
| 2548 | RBD | BG4NUC-LC317 |
| 2549 | RBD | BG4NUC-LC318 |
| 2550 | 2P | BG4NUC-LC319 |
| 2551 | 2P | BG4NUC-LC320 |
| 2552 | 2P | BG4NUC-LC321 |
| 2553 | RBD | BG4NUC-LC322 |
| 2554 | 2P | BG4NUC-LC323 |
| 2555 | RBD | BG4NUC-LC324 |
| 2556 | 2P | BG4NUC-LC325 |
| 2557 | RBD | BG4NUC-LC326 |
| 2558 | RBD | BG4NUC-LC327 |
| 2559 | RBD | BG4NUC-LC328 |
| 2560 | 2P | BG4NUC-LC329 |
| 2561 | 2P | BG4NUC-LC330 |
| 2562 | RBD | BG4NUC-LC331 |
| 2563 | 2P | BG4NUC-LC332 |
| 2564 | 2P | BG4NUC-LC333 |
| 2565 | 2P | BG4NUC-LC334 |
| 2566 | 2F | BG4NUC-LC335 |
| 2567 | RBD | BG4NUC-LC336 |
| 2568 | 2P | BG4NUC-LC337 |
| 2569 | RBD | BG4NUC-LC338 |
| 2570 | 2P | BG4NUC-LC339 |
| 2571 | RBD | BG4NUC-LC340 |
| 2572 | 2P | BG4NUC-LC341 |
| 2573 | RBD | BG4NUC-LC342 |
| 2574 | RBD | BG4NUC-LC343 |
| 2575 | RBD | BG4NUC-LC344 |
| 2576 | 2P | BG4NUC-LC345 |
| 2577 | RBD | BG4NUC-LC346 |
| 2578 | 2P | BG4NUC-LC347 |
| 2579 | RBD | BG4NUC-LC348 |
| 2580 | 2P | BG4NUC-LC349 |
| 2581 | 2P | BG4NUC-LC350 |
| 2582 | 2P | BG4NUC-LC351 |
| 2583 | 2P | BG4NUC-LC352 |
| 2584 | RBD | BG4NUC-LC353 |
| 2585 | RBD | BG4NUC-LC354 |
| 2586 | RBD | BG4NUC-LC355 |
| 2587 | 2P | BG4NUC-LC356 |
| 2588 | 2P | BG4NUC-LC357 |
| 2589 | RBD | BG4NUC-LC358 |
| 2590 | RBD | BG4NUC-LC359 |
| 2591 | RBD | BG4NUC-LC360 |
| 2592 | RBD | BG4NUC-LC361 |
| 2593 | RBD | BG4NUC-LC362 |
| 2594 | 2P | BG4NUC-LC363 |
| 2595 | 2P | BG4NUC-LC364 |
| 2596 | RBD | BG4NUC-LC365 |
| 2597 | 2P | BG4NUC-LC366 |
| 2598 | 2P | BG4NUC-LC367 |
| 2599 | 2P | BG4NUC-LC368 |
| 2600 | 2P | BG4NUC-LC369 |
| 2601 | 2P | BG4NUC-LC370 |
| 2602 | 2P | BG4NUC-LC371 |
| 2603 | 2P | BG4NUC-LC372 |
| 2604 | 2P | BG4NUC-LC373 |
| 2605 | 2P | BG4NUC-LC374 |
| 2606 | RBD | BG4NUC-LC375 |
| 2607 | 2P | BG4NUC-LC376 |
| 2608 | 2P | BG4NUC-LC377 |
| 2609 | 2P | BG4NUC-LC378 |
| 2610 | RBD | BG4NUC-LC379 |
| 2611 | 2P | BG4NUC-LC380 |
| 2612 | 2P | BG4NUC-LC381 |
| 2613 | 2P | BG4NUC-LC382 |
| 2614 | 2P | BG4NUC-LC383 |
| 2615 | 2P | BG4NUC-LC384 |
| 2616 | 2P | BG4NUC-LC385 |
| 2617 | RBD | BG4NUC-LC386 |
| 2618 | RBD | BG4NUC-LC387 |
| 2619 | 2P | BG4NUC-LC388 |
| 2620 | 2P | BG4NUC-LC389 |
| 2621 | 2P | BG4NUC-LC390 |
| 2622 | 2P | BG4NUC-LC391 |
| 2623 | 2P | BG4NUC-LC392 |
| 2624 | 2P | BG4NUC-LC393 |
| 2625 | RBD | BG4NUC-LC394 |
| 2626 | 2P | BG4NUC-LC395 |
| 2627 | 2P | BG4NUC-LC396 |
| 2628 | 2P | BG4NUC-LC397 |
| 2629 | 2P | BG4NUC-LC398 |
| 2630 | 2P | BG4NUC-LC399 |
| 2631 | RBD | BG4NUC-LC400 |
| 2632 | RBD | BG4NUC-LC401 |
| 2633 | 2P | BG4NUC-LC402 |
| 2634 | 2P | BG4NUC-LC403 |
| 2635 | 2P | BG4NUC-LC404 |
| 2636 | RBD | BG4NUC-LC405 |
| 2637 | 2P | BG4NUC-LC406 |
| 2638 | RBD | BG4NUC-LC407 |
| 2639 | 2P | BG4NUC-LC408 |
| 2640 | 2P | BG4NUC-LC409 |
| 2641 | 2P | BG5NUC-HC1 |
| 2642 | RBD | BG5NUC-HC2 |
| 2643 | RBD | BG5NUC-HC3 |
| 2644 | 2P | BG5NUC-HC4 |
| 2645 | RBD | BG5NUC-HC5 |
| 2646 | 2P | BG5NUC-HC6 |
| 2647 | RBD | BG5NUC-HC7 |
| 2648 | 2P | BG5NUC-HC8 |
| 2649 | RBD | BG5NUC-HC9 |
| 2650 | 2P | BG5NUC-HC10 |
| 2651 | RBD | BG5NUC-HC11 |
| 2652 | 2P | BG5NUC-HC12 |
| 2653 | 2P | BG5NUC-HC13 |
| 2654 | 2P | BG5NUC-HC14 |
| 2655 | 2P | BG5NUC-HC15 |
| 2656 | RBD | BG5NUC-HC16 |
| 2657 | RBD | BG5NUC-HC17 |
| 2658 | 2P | BG5NUC-HC18 |
| 2659 | RBD | BG5NUC-HC19 |
| 2660 | RBD | BG5NUC-HC20 |
| 2661 | 2P | BG5NUC-HC21 |
| 2662 | RBD | BG5NUC-HC22 |
| 2663 | 2P | BG5NUC-HC23 |
| 2664 | RBD | BG5NUC-HC24 |
| 2665 | RBD | BG5NUC-HC25 |
| 2666 | 2F | BG5NUC-HC26 |
| 2667 | RBD | BG5NUC-HC27 |
| 2668 | RBD | BG5NUC-HC28 |
| 2669 | RBD | BG5NUC-HC29 |
| 2670 | RBD | BG5NUC-HC30 |
| 2671 | RBD | BG5NUC-HC31 |
| 2672 | 2P | BG5NUC-HC32 |
| 2673 | RBD | BG5NUC-HC33 |
| 2674 | RBD | BG5NUC-HC34 |
| 2675 | 2P | BG5NUC-HC35 |
| 2676 | RBD | BG5NUC-HC36 |
| 2677 | RBD | BG5NUC-HC37 |
| 2678 | 2P | BG5NUC-HC38 |
| 2679 | 2P | BG5NUC-HC39 |
| 2680 | 2P | BG5NUC-HC40 |
| 2681 | 2P | BG5NUC-HC41 |
| 2682 | 2P | BG5NUC-HC42 |
| 2683 | 2P | BG5NUC-HC43 |
| 2684 | RBD | BG5NUC-HC44 |
| 2685 | 2P | BG5NUC-HC45 |
| 2686 | 2P | BG5NUC-HC46 |
| 2687 | RBD | BG5NUC-HC47 |
| 2688 | RBD | BG5NUC-HC48 |
| 2689 | RBD | BG5NUC-HC49 |
| 2690 | RBD | BG5NUC-HC50 |
| 2691 | 2P | BG5NUC-HC51 |
| 2692 | 2P | BG5NUC-HC52 |
| 2693 | 2P | BG5NUC-HC53 |
| 2694 | 2P | BG5NUC-HC54 |
| 2695 | 2P | BG5NUC-HC55 |
| 2696 | 2P | BG5NUC-HC56 |
| 2697 | 2P | BG5NUC-HC57 |
| 2698 | 2P | BG5NUC-HC58 |
| 2699 | 2P | BG5NUC-HC59 |
| 2700 | 2P | BG5NUC-HC60 |
| 2701 | RBD | BG5NUC-HC61 |
| 2702 | RBD | BG5NUC-HC62 |
| 2703 | RBD | BG5NUC-HC63 |
| 2704 | RBD | BG5NUC-HC64 |
| 2705 | RBD | BG5NUC-HC65 |
| 2706 | RBD | BG5NUC-HC66 |
| 2707 | RBD | BG5NUC-HC67 |
| 2708 | RBD | BG5NUC-HC68 |
| 2709 | RBD | BG5NUC-HC69 |
| 2710 | RBD | BG5NUC-HC70 |
| 2711 | 2P | BG5NUC-HC71 |
| 2712 | RBD | BG5NUC-HC72 |
| 2713 | 2P | BG5NUC-HC73 |
| 2714 | 2P | BG5NUC-HC74 |
| 2715 | 2P | BG5NUC-HC75 |
| 2716 | 2P | BG5NUC-HC76 |
| 2717 | 2P | BG5NUC-HC77 |
| 2718 | 2P | BG5NUC-HC78 |
| 2719 | 2P | BG5NUC-HC79 |
| 2720 | 2P | BG5NUC-HC80 |
| 2721 | 2P | BG5NUC-HC81 |
| 2722 | 2P | BG5NUC-HC82 |
| 2723 | 2P | BG5NUC-HC83 |
| 2724 | 2P | BG5NUC-HC84 |
| 2725 | 2P | BG5NUC-HC85 |
| 2726 | 2P | BG5NUC-HC86 |
| 2727 | 2P | BG5NUC-HC87 |
| 2728 | 2P | BG5NUC-HC88 |
| 2729 | 2P | BG5NUC-HC89 |
| 2730 | 2P | BG5NUC-HC90 |
| 2731 | 2P | BG5NUC-HC91 |
| 2732 | 2P | BG5NUC-HC92 |
| 2733 | 2P | BG5NUC-HC93 |
| 2734 | 2P | BG5NUC-HC94 |
| 2735 | 2P | BG5NUC-HC95 |
| 2736 | 2P | BG5NUC-HC96 |
| 2737 | RBD | BG5NUC-HC97 |
| 2738 | RBD | BG5NUC-HC98 |
| 2739 | RBD | BG5NUC-HC99 |
| 2740 | RBD | BG5NUC-HC100 |
| 2741 | 2P | BG5NUC-HC101 |
| 2742 | 2P | BG5NUC-HC102 |
| 2743 | 2P | BG5NUC-HC103 |
| 2744 | 2P | BG5NUC-HC104 |
| 2745 | RBD | BG5NUC-HC105 |
| 2746 | RBD | BG5NUC-HC106 |
| 2747 | 2P | BG5NUC-HC107 |
| 2748 | RBD | BG5NUC-HC108 |
| 2749 | 2P | BG5NUC-HC109 |
| 2750 | RBD | BG5NUC-HC110 |
| 2751 | 2P | BG5NUC-HC111 |
| 2752 | RBD | BG5NUC-HC112 |
| 2753 | 2P | BG5NUC-HC113 |
| 2754 | 2P | BG5NUC-HC114 |
| 2755 | 2P | BG5NUC-HC115 |
| 2756 | 2P | BG5NUC-HC116 |
| 2757 | RBD | BG5NUC-HC117 |
| 2758 | 2P | BG5NUC-HC118 |
| 2759 | 2P | BG5NUC-HC119 |
| 2760 | 2P | BG5NUC-HC120 |
| 2761 | RBD | BG5NUC-HC121 |
| 2762 | RBD | BG5NUC-HC122 |
| 2763 | 2P | BG5NUC-HC123 |
| 2764 | 2P | BG5NUC-HC124 |
| 2765 | 2P | BG5NUC-HC125 |
| 2766 | 2P | BG5NUC-HC126 |
| 2767 | 2P | BG5NUC-HC127 |
| 2768 | 2P | BG5NUC-HC128 |
| 2769 | RBD | BG5NUC-HC129 |
| 2770 | 2P | BG5NUC-HC130 |
| 2771 | RBD | BG5NUC-HC131 |
| 2772 | 2P | BG5NUC-HC132 |
| 2773 | RBD | BG5NUC-HC133 |
| 2774 | RBD | BG5NUC-HC134 |
| 2775 | 2P | BG5NUC-HC135 |
| 2776 | 2P | BG5NUC-HC136 |
| 2777 | 2P | BG5NUC-HC137 |
| 2778 | 2P | BG5NUC-HC138 |
| 2779 | 2P | BG5NUC-HC139 |
| 2780 | 2P | BG5NUC-HC140 |
| 2781 | 2P | BG5NUC-HC141 |
| 2782 | 2P | BG5NUC-HC142 |
| 2783 | 2P | BG5NUC-HC143 |
| 2784 | RBD | BG5NUC-HC144 |
| 2785 | 2P | BG5NUC-HC145 |
| 2786 | 2P | BG5NUC-HC146 |
| 2787 | 2P | BG5NUC-HC147 |
| 2788 | 2P | BG5NUC-HC148 |
| 2789 | 2P | BG5NUC-HC149 |
| 2790 | 2P | BG5NUC-HC150 |
| 2791 | 2P | BG5NUC-HC151 |
| 2792 | 2P | BG5NUC-HC152 |
| 2793 | 2P | BG5NUC-HC153 |
| 2794 | 2P | BG5NUC-HC154 |
| 2795 | 2P | BG5NUC-HC155 |
| 2796 | RBD | BG5NUC-HC156 |
| 2797 | RBD | BG5NUC-HC157 |
| 2798 | 2P | BG5NUC-HC158 |
| 2799 | 2P | BG5NUC-HC159 |
| 2800 | 2P | BG5NUC-HC160 |
| 2801 | 2P | BG5NUC-HC161 |
| 2802 | 2P | BG5NUC-HC162 |
| 2803 | RBD | BG5NUC-HC163 |
| 2804 | 2P | BG5NUC-HC164 |
| 2805 | 2P | BG5NUC-HC165 |
| 2806 | RBD | BG5NUC-HC166 |
| 2807 | 2P | BG5NUC-HC167 |
| 2808 | 2P | BG5NUC-HC168 |
| 2809 | 2P | BG5NUC-HC169 |
| 2810 | RBD | BG5NUC-HC170 |
| 2811 | RBD | BG5NUC-HC171 |
| 2812 | RBD | BG5NUC-HC172 |
| 2813 | 2P | BG5NUC-HC173 |
| 2814 | 2P | BG5NUC-HC174 |
| 2815 | 2P | BG5NUC-HC175 |
| 2816 | 2P | BG5NUC-HC176 |
| 2817 | 2P | BG5NUC-HC177 |
| 2818 | 2P | BG5NUC-HC178 |
| 2819 | RBD | BG5NUC-HC179 |
| 2820 | RBD | BG5NUC-HC180 |
| 2821 | 2P | BG5NUC-HC181 |
| 2822 | 2P | BG5NUC-HC182 |
| 2823 | 2P | BG5NUC-HC183 |
| 2824 | 2P | BG5NUC-HC184 |
| 2825 | 2P | BG5NUC-HC185 |
| 2826 | 2P | BG5NUC-HC186 |
| 2827 | 2P | BG5NUC-HC187 |
| 2828 | 2P | BG5NUC-HC188 |
| 2829 | 2P | BG5NUC-HC189 |
| 2830 | 2P | BG5NUC-HC190 |
| 2831 | 2P | BG5NUC-HC191 |
| 2832 | 2P | BG5NUC-HC192 |
| 2833 | 2P | BG5NUC-HC193 |
| 2834 | 2P | BG5NUC-HC194 |
| 2835 | 2P | BG5NUC-HC195 |
| 2836 | 2P | BG5NUC-HC196 |
| 2837 | RBD | BG5NUC-HC197 |
| 2838 | 2P | BG5NUC-HC198 |
| 2839 | 2P | BG5NUC-HC199 |
| 2840 | RBD | BG5NUC-HC200 |
| 2841 | 2P | BG5NUC-HC201 |
| 2842 | RBD | BG5NUC-HC202 |
| 2843 | 2P | BG5NUC-HC203 |
| 2844 | 2P | BG5NUC-HC204 |
| 2845 | 2P | BG5NUC-HC205 |
| 2846 | RBD | BG5NUC-HC206 |
| 2847 | 2P | BG5NUC-HC207 |
| 2848 | RBD | BG5NUC-HC208 |
| 2849 | 2P | BG5NUC-HC209 |
| 2850 | 2P | BG5NUC-HC210 |
| 2851 | RBD | BG5NUC-HC211 |
| 2852 | 2P | BG5NUC-HC212 |
| 2853 | 2P | BG5NUC-HC213 |
| 2854 | 2P | BG5NUC-HC214 |
| 2855 | 2P | BG5NUC-HC215 |
| 2856 | RBD | BG5NUC-HC216 |
| 2857 | RBD | BG5NUC-HC217 |
| 2858 | 2P | BG5NUC-HC218 |
| 2859 | RBD | BG5NUC-HC219 |
| 2860 | 2P | BG5NUC-HC220 |
| 2861 | 2P | BG5NUC-HC221 |
| 2862 | 2P | BG5NUC-HC222 |
| 2863 | RBD | BG5NUC-HC223 |
| 2864 | 2P | BG5NUC-HC224 |
| 2865 | 2P | BG5NUC-HC225 |
| 2866 | 2P | BG5NUC-HC226 |
| 2867 | RBD | BG5NUC-HC227 |
| 2868 | 2P | BG5NUC-HC228 |
| 2869 | 2P | BG5NUC-HC229 |
| 2870 | RBD | BG5NUC-HC230 |
| 2871 | RBD | BG5NUC-HC231 |
| 2872 | 2P | BG5NUC-HC232 |
| 2873 | 2P | BG5NUC-HC233 |
| 2874 | 2P | BG5NUC-HC234 |
| 2875 | 2P | BG5NUC-HC235 |
| 2876 | 2P | BG5NUC-HC236 |
| 2877 | 2P | BG5NUC-HC237 |
| 2878 | 2P | BG5NUC-HC238 |
| 2879 | 2P | BG5NUC-HC239 |
| 2880 | RBD | BG5NUC-HC240 |
| 2881 | RBD | BG5NUC-HC241 |
| 2882 | RBD | BG5NUC-HC242 |
| 2883 | RBD | BG5NUC-HC243 |
| 2884 | 2P | BG5NUC-HC244 |
| 2885 | 2P | BG5NUC-HC245 |
| 2886 | 2P | BG5NUC-HC246 |
| 2887 | RBD | BG5NUC-HC247 |
| 2888 | RBD | BG5NUC-HC248 |
| 2889 | RBD | BG5NUC-HC249 |
| 2890 | 2P | BG5NUC-HC250 |
| 2891 | 2P | BG5NUC-HC251 |
| 2892 | 2P | BG5NUC-HC252 |
| 2893 | 2P | BG5NUC-HC253 |
| 2894 | 2P | BG5NUC-HC254 |
| 2895 | 2P | BG5NUC-HC255 |
| 2896 | RBD | BG5NUC-HC256 |
| 2897 | 2P | BG5NUC-HC257 |
| 2898 | 2P | BG5NUC-HC258 |
| 2899 | 2P | BG5NUC-HC259 |
| 2900 | 2P | BG5NUC-HC260 |
| 2901 | 2P | BG5NUC-HC261 |
| 2902 | 2P | BG5NUC-HC262 |
| 2903 | 2P | BG5NUC-HC263 |
| 2904 | 2P | BG5NUC-HC264 |
| 2905 | 2P | BG5NUC-HC265 |
| 2906 | 2F | BG5NUC-HC266 |
| 2907 | RBD | BG5NUC-HC267 |
| 2908 | 2P | BG5NUC-HC268 |
| 2909 | RBD | BG5NUC-HC269 |
| 2910 | RBD | BG5NUC-HC270 |
| 2911 | 2P | BG5NUC-HC271 |
| 2912 | 2P | BG5NUC-HC272 |
| 2913 | RBD | BG5NUC-HC273 |
| 2914 | 2P | BG5NUC-HC274 |
| 2915 | 2P | BG5NUC-HC275 |
| 2916 | 2P | BG5NUC-HC276 |
| 2917 | RBD | BG5NUC-HC277 |
| 2918 | 2P | BG5NUC-HC278 |
| 2919 | RBD | BG5NUC-HC279 |
| 2920 | RBD | BG5NUC-HC280 |
| 2921 | 2P | BG5NUC-HC281 |
| 2922 | 2P | BG5NUC-HC282 |
| 2923 | RBD | BG5NUC-HC283 |
| 2924 | RBD | BG5NUC-HC284 |
| 2925 | RBD | BG5NUC-HC285 |
| 2926 | 2P | BG5NUC-HC286 |
| 2927 | 2P | BG5NUC-HC287 |
| 2928 | 2P | BG5NUC-HC288 |
| 2929 | 2P | BG5NUC-HC289 |
| 2930 | 2P | BG5NUC-HC290 |
| 2931 | 2P | BG5NUC-HC291 |
| 2932 | 2P | BG5NUC-HC292 |
| 2933 | 2P | BG5NUC-HC293 |
| 2934 | 2P | BG5NUC-HC294 |
| 2935 | 2P | BG5NUC-HC295 |
| 2936 | RBD | BG5NUC-HC296 |
| 2937 | RBD | BG5NUC-HC297 |
| 2938 | 2P | BG5NUC-HC298 |
| 2939 | RBD | BG5NUC-HC299 |
| 2940 | RBD | BG5NUC-HC300 |
| 2941 | 2P | BG5NUC-HC301 |
| 2942 | 2P | BG5NUC-HC302 |
| 2943 | RBD | BG5NUC-HC303 |
| 2944 | 2P | BG5NUC-HC304 |
| 2945 | RBD | BG5NUC-HC305 |
| 2946 | RBD | BG5NUC-HC306 |
| 2947 | RBD | BG5NUC-HC307 |
| 2948 | 2P | BG5NUC-HC308 |
| 2949 | 2P | BG5NUC-HC309 |
| 2950 | RBD | BG5NUC-HC310 |
| 2951 | 2P | BG5NUC-HC311 |
| 2952 | 2P | BG5NUC-HC312 |
| 2953 | 2P | BG5NUC-HC313 |
| 2954 | 2P | BG5NUC-HC314 |
| 2955 | RBD | BG5NUC-HC315 |
| 2956 | 2P | BG5NUC-HC316 |
| 2957 | RBD | BG5NUC-HC317 |
| 2958 | RBD | BG5NUC-HC318 |
| 2959 | 2P | BG5NUC-HC319 |
| 2960 | RBD | BG5NUC-HC320 |
| 2961 | RBD | BG5NUC-HC321 |
| 2962 | RBD | BG5NUC-HC322 |
| 2963 | 2P | BG5NUC-HC323 |
| 2964 | 2P | BG5NUC-HC324 |
| 2965 | 2P | BG5NUC-HC325 |
| 2966 | RBD | BG5NUC-HC326 |
| 2967 | 2P | BG5NUC-HC327 |
| 2968 | 2P | BG5NUC-HC328 |
| 2969 | 2P | BG5NUC-HC329 |
| 2970 | 2P | BG5NUC-HC330 |
| 2971 | 2P | BG5NUC-HC331 |
| 2972 | RBD | BG5NUC-HC332 |
| 2973 | RBD | BG5NUC-HC333 |
| 2974 | RBD | BG5NUC-HC334 |
| 2975 | RBD | BG5NUC-HC335 |
| 2976 | RBD | BG5NUC-HC336 |
| 2977 | RBD | BG5NUC-HC337 |
| 2978 | 2P | BG5NUC-HC338 |
| 2979 | 2P | BG5NUC-HC339 |
| 2980 | 2P | BG5NUC-HC340 |
| 2981 | 2P | BG5NUC-HC341 |
| 2982 | 2P | BG5NUC-HC342 |
| 2983 | 2P | BG5NUC-HC343 |
| 2984 | 2P | BG5NUC-HC344 |
| 2985 | 2P | BG5NUC-HC345 |
| 2986 | RBD | BG5NUC-HC346 |
| 2987 | RBD | BG5NUC-HC347 |
| 2988 | 2P | BG5NUC-HC348 |
| 2989 | 2F | BG5NUC-HC349 |
| 2990 | 2P | BG5NUC-HC350 |
| 2991 | 2P | BG5NUC-HC351 |
| 2992 | RBD | BG5NUC-HC352 |
| 2993 | 2P | BG5NUC-HC353 |
| 2994 | RBD | BG5NUC-HC354 |
| 2995 | RBD | BG5NUC-HC355 |
| 2996 | 2P | BG5NUC-HC356 |
| 2997 | RBD | BG5NUC-HC357 |
| 2998 | 2P | BG5NUC-HC358 |
| 2999 | RBD | BG5NUC-HC359 |
| 3000 | 2P | BG5NUC-HC360 |
| 3001 | 2P | BG5NUC-HC361 |
| 3002 | 2P | BG5NUC-HC362 |
| 3003 | RBD | BG5NUC-HC363 |
| 3004 | 2P | BG5NUC-HC364 |
| 3005 | 2P | BG5NUC-HC365 |
| 3006 | 2P | BG5NUC-HC366 |
| 3007 | 2P | BG5NUC-HC367 |
| 3008 | RBD | BG5NUC-HC368 |
| 3009 | RBD | BG5NUC-HC369 |
| 3010 | 2P | BG5NUC-HC370 |
| 3011 | RBD | BG5NUC-HC371 |
| 3012 | 2P | BG5NUC-HC372 |
| 3013 | RBD | BG5NUC-HC373 |
| 3014 | 2P | BG5NUC-HC374 |
| 3015 | 2P | BG5NUC-HC375 |
| 3016 | 2P | BG5NUC-HC376 |
| 3017 | RBD | BG5NUC-HC377 |
| 3018 | 2P | BG5NUC-HC378 |
| 3019 | 2P | BG5NUC-HC379 |
| 3020 | RBD | BG5NUC-HC380 |
| 3021 | RBD | BG5NUC-HC381 |
| 3022 | RBD | BG5NUC-HC382 |
| 3023 | RBD | BG5NUC-HC383 |
| 3024 | 2P | BG5NUC-HC384 |
| 3025 | 2P | BG5NUC-HC385 |
| 3026 | 2F | BG5NUC-HC386 |
| 3027 | 2P | BG5NUC-HC387 |
| 3028 | 2F | BG5NUC-HC388 |
| 3029 | 2P | BG5NUC-HC389 |
| 3030 | 2P | BG5NUC-HC390 |
| 3031 | 2P | BG5NUC-HC391 |
| 3032 | 2P | BG5NUC-HC392 |
| 3033 | 2P | BG5NUC-HC393 |
| 3034 | RBD | BG5NUC-HC394 |
| 3035 | RBD | BG5NUC-HC395 |
| 3036 | RBD | BG5NUC-HC396 |
| 3037 | 2P | BG5NUC-HC397 |
| 3038 | 2P | BG5NUC-HC398 |
| 3039 | 2P | BG5NUC-HC399 |
| 3040 | 2P | BG5NUC-HC400 |
| 3041 | 2P | BG5NUC-HC401 |
| 3042 | 2P | BG5NUC-HC402 |
| 3043 | 2F | BG5NUC-HC403 |
| 3044 | 2P | BG5NUC-HC404 |
| 3045 | 2P | BG5NUC-HC405 |
| 3046 | 2P | BG5NUC-HC406 |
| 3047 | RBD | BG5NUC-HC407 |
| 3048 | 2P | BG5NUC-HC408 |
| 3049 | RBD | BG5NUC-HC409 |
| 3050 | RBD | BG5NUC-HC410 |
| 3051 | 2P | BG5NUC-HC411 |
| 3052 | 2P | BG5NUC-HC412 |
| 3053 | 2P | BG5NUC-HC413 |
| 3054 | 2P | BG5NUC-HC414 |
| 3055 | 2P | BG5NUC-HC415 |
| 3056 | RBD | BG5NUC-HC416 |
| 3057 | 2P | BG5NUC-HC417 |
| 3058 | RBD | BG5NUC-HC418 |
| 3059 | 2P | BG5NUC-HC419 |
| 3060 | 2P | BG5NUC-HC420 |
| 3061 | 2P | BG5NUC-HC421 |
| 3062 | 2P | BG5NUC-HC422 |
| 3063 | 2P | BG5NUC-HC423 |
| 3064 | 2P | BG5NUC-HC424 |
| 3065 | RBD | BG5NUC-HC425 |
| 3066 | 2P | BG5NUC-HC426 |
| 3067 | RBD | BG5NUC-HC427 |
| 3068 | 2P | BG5NUC-HC428 |
| 3069 | 2P | BG5NUC-HC429 |
| 3070 | 2P | BG5NUC-HC430 |
| 3071 | 2P | BG5NUC-HC431 |
| 3072 | 2P | BG5NUC-HC432 |
| 3073 | 2P | BG5NUC-HC433 |
| 3074 | RBD | BG5NUC-HC434 |
| 3075 | RBD | BG5NUC-HC435 |
| 3076 | 2P | BG5NUC-HC436 |
| 3077 | RBD | BG5NUC-HC437 |
| 3078 | 2P | BG5NUC-HC438 |
| 3079 | 2P | BG5NUC-HC439 |
| 3080 | 2P | BG5NUC-HC440 |
| 3081 | 2P | BG5NUC-HC441 |
| 3082 | RBD | BG5NUC-HC442 |
| 3083 | 2P | BG5NUC-HC443 |
| 3084 | 2P | BG5NUC-HC444 |
| 3085 | 2P | BG5NUC-HC445 |
| 3086 | 2P | BG5NUC-HC446 |
| 3087 | 2P | BG5NUC-HC447 |
| 3088 | RBD | BG5NUC-HC448 |
| 3089 | 2P | BG5NUC-HC449 |
| 3090 | 2P | BG5NUC-HC450 |
| 3091 | 2P | BG5NUC-HC451 |
| 3092 | 2P | BG5NUC-HC452 |
| 3093 | 2P | BG5NUC-HC453 |
| 3094 | 2P | BG5NUC-HC454 |
| 3095 | 2P | BG5NUC-HC455 |
| 3096 | 2P | BG5NUC-HC456 |
| 3097 | 2P | BG5NUC-HC457 |
| 3098 | 2P | BG5NUC-HC458 |
| 3099 | 2P | BG5NUC-HC459 |
| 3100 | 2P | BG5NUC-HC460 |
| 3101 | RBD | BG5NUC-HC461 |
| 3102 | 2P | BG5NUC-HC462 |
| 3103 | 2P | BG5NUC-HC463 |
| 3104 | 2P | BG5NUC-HC464 |
| 3105 | 2P | BG5NUC-HC465 |
| 3106 | RBD | BG5NUC-HC466 |
| 3107 | 2P | BG5NUC-HC467 |
| 3108 | 2P | BG5NUC-HC468 |
| 3109 | 2P | BG5NUC-HC469 |
| 3110 | RBD | BG5NUC-HC470 |
| 3111 | RBD | BG5NUC-HC471 |
| 3112 | 2F | BG5NUC-HC472 |
| 3113 | RBD | BG5NUC-HC473 |
| 3114 | RBD | BG5NUC-HC474 |
| 3115 | RBD | BG5NUC-HC475 |
| 3116 | 2P | BG5NUC-HC476 |
| 3117 | 2P | BG5NUC-LC1 |
| 3118 | RBD | BG5NUC-LC2 |
| 3119 | RBD | BG5NUC-LC3 |
| 3120 | 2P | BG5NUC-LC4 |
| 3121 | RBD | BG5NUC-LC5 |
| 3122 | 2P | BG5NUC-LC6 |
| 3123 | RBD | BG5NUC-LC7 |
| 3124 | 2P | BG5NUC-LC8 |
| 3125 | RBD | BG5NUC-LC9 |
| 3126 | 2P | BG5NUC-LC10 |
| 3127 | RBD | BG5NUC-LC11 |
| 3128 | 2P | BG5NUC-LC12 |
| 3129 | 2P | BG5NUC-LC13 |
| 3130 | 2P | BG5NUC-LC14 |
| 3131 | 2P | BG5NUC-LC15 |
| 3132 | RBD | BG5NUC-LC16 |
| 3133 | RBD | BG5NUC-LC17 |
| 3134 | 2P | BG5NUC-LC18 |
| 3135 | RBD | BG5NUC-LC19 |
| 3136 | RBD | BG5NUC-LC20 |
| 3137 | 2P | BG5NUC-LC21 |
| 3138 | RBD | BG5NUC-LC22 |
| 3139 | 2P | BG5NUC-LC23 |
| 3140 | RBD | BG5NUC-LC24 |
| 3141 | RBD | BG5NUC-LC25 |
| 3142 | 2P | BG5NUC-LC26 |
| 3143 | RBD | BG5NUC-LC27 |
| 3144 | RBD | BG5NUC-LC28 |
| 3145 | RBD | BG5NUC-LC29 |
| 3146 | RBD | BG5NUC-LC30 |
| 3147 | RBD | BG5NUC-LC31 |
| 3148 | 2P | BG5NUC-LC32 |
| 3149 | RBD | BG5NUC-LC33 |
| 3150 | RBD | BG5NUC-LC34 |
| 3151 | 2P | BG5NUC-LC35 |
| 3152 | RBD | BG5NUC-LC36 |
| 3153 | RBD | BG5NUC-LC37 |
| 3154 | 2P | BG5NUC-LC38 |
| 3155 | 2P | BG5NUC-LC39 |
| 3156 | 2P | BG5NUC-LC40 |
| 3157 | 2P | BG5NUC-LC41 |
| 3158 | 2P | BG5NUC-LC42 |
| 3159 | 2P | BG5NUC-LC43 |
| 3160 | RBD | BG5NUC-LC44 |
| 3161 | 2P | BG5NUC-LC45 |
| 3162 | 2P | BG5NUC-LC46 |
| 3163 | RBD | BG5NUC-LC47 |
| 3164 | RBD | BG5NUC-LC48 |
| 3165 | RBD | BG5NUC-LC49 |
| 3166 | RBD | BG5NUC-LC50 |
| 3167 | 2P | BG5NUC-LC51 |
| 3168 | 2P | BG5NUC-LC52 |
| 3169 | 2P | BG5NUC-LC53 |
| 3170 | 2P | BG5NUC-LC54 |
| 3171 | 2P | BG5NUC-LC55 |
| 3172 | 2P | BG5NUC-LC56 |
| 3173 | 2P | BG5NUC-LC57 |
| 3174 | 2P | BG5NUC-LC58 |
| 3175 | 2P | BG5NUC-LC59 |
| 3176 | 2P | BG5NUC-LC60 |
| 3177 | RBD | BG5NUC-LC61 |
| 3178 | RBD | BG5NUC-LC62 |
| 3179 | RBD | BG5NUC-LC63 |
| 3180 | RBD | BG5NUC-LC64 |
| 3181 | RBD | BG5NUC-LC65 |
| 3182 | RBD | BG5NUC-LC66 |
| 3183 | RBD | BG5NUC-LC67 |
| 3184 | RBD | BG5NUC-LC68 |
| 3185 | RBD | BG5NUC-LC69 |
| 3186 | RBD | BG5NUC-LC70 |
| 3187 | 2P | BG5NUC-LC71 |
| 3188 | RBD | BG5NUC-LC72 |
| 3189 | 2P | BG5NUC-LC73 |
| 3190 | 2P | BG5NUC-LC74 |
| 3191 | 2P | BG5NUC-LC75 |
| 3192 | 2P | BG5NUC-LC76 |
| 3193 | 2P | BG5NUC-LC77 |
| 3194 | 2P | BG5NUC-LC78 |
| 3195 | 2P | BG5NUC-LC79 |
| 3196 | 2P | BG5NUC-LC80 |
| 3197 | 2P | BG5NUC-LC81 |
| 3198 | 2P | BG5NUC-LC82 |
| 3199 | 2P | BG5NUC-LC83 |
| 3200 | 2P | BG5NUC-LC84 |
| 3201 | 2P | BG5NUC-LC85 |
| 3202 | 2P | BG5NUC-LC86 |
| 3203 | 2P | BG5NUC-LC87 |
| 3204 | 2P | BG5NUC-LC88 |
| 3205 | 2P | BG5NUC-LC89 |
| 3206 | 2P | BG5NUC-LC90 |
| 3207 | 2P | BG5NUC-LC91 |
| 3208 | 2P | BG5NUC-LC92 |
| 3209 | 2P | BG5NUC-LC93 |
| 3210 | 2P | BG5NUC-LC94 |
| 3211 | 2P | BG5NUC-LC95 |
| 3212 | 2P | BG5NUC-LC96 |
| 3213 | RBD | BG5NUC-LC97 |
| 3214 | RBD | BG5NUC-LC98 |
| 3215 | RBD | BG5NUC-LC99 |
| 3216 | RBD | BG5NUC-LC100 |
| 3217 | 2P | BG5NUC-LC101 |
| 3218 | 2P | BG5NUC-LC102 |
| 3219 | 2P | BG5NUC-LC103 |
| 3220 | 2P | BG5NUC-LC104 |
| 3221 | RBD | BG5NUC-LC105 |
| 3222 | RBD | BG5NUC-LC106 |
| 3223 | 2P | BG5NUC-LC107 |
| 3224 | RBD | BG5NUC-LC108 |
| 3225 | 2P | BG5NUC-LC109 |
| 3226 | RBD | BG5NUC-LC110 |
| 3227 | 2P | BG5NUC-LC111 |
| 3228 | RBD | BG5NUC-LC112 |
| 3229 | 2P | BG5NUC-LC113 |
| 3230 | 2P | BG5NUC-LC114 |
| 3231 | 2P | BG5NUC-LC115 |
| 3232 | 2P | BG5NUC-LC116 |
| 3233 | RBD | BG5NUC-LC117 |
| 3234 | 2P | BG5NUC-LC118 |
| 3235 | 2P | BG5NUC-LC119 |
| 3236 | 2P | BG5NUC-LC120 |
| 3237 | RBD | BG5NUC-LC121 |
| 3238 | RBD | BG5NUC-LC122 |
| 3239 | 2P | BG5NUC-LC123 |
| 3240 | 2P | BG5NUC-LC124 |
| 3241 | 2P | BG5NUC-LC125 |
| 3242 | 2P | BG5NUC-LC126 |
| 3243 | 2P | BG5NUC-LC127 |
| 3244 | 2P | BG5NUC-LC128 |
| 3245 | RBD | BG5NUC-LC129 |
| 3246 | 2P | BG5NUC-LC130 |
| 3247 | RBD | BG5NUC-LC131 |
| 3248 | 2P | BG5NUC-LC132 |
| 3249 | RBD | BG5NUC-LC133 |
| 3250 | RBD | BG5NUC-LC134 |
| 3251 | 2P | BG5NUC-LC135 |
| 3252 | 2P | BG5NUC-LC136 |
| 3253 | 2P | BG5NUC-LC137 |
| 3254 | 2P | BG5NUC-LC138 |
| 3255 | 2P | BG5NUC-LC139 |
| 3256 | 2P | BG5NUC-LC140 |
| 3257 | 2P | BG5NUC-LC141 |
| 3258 | 2P | BG5NUC-LC142 |
| 3259 | 2P | BG5NUC-LC143 |
| 3260 | RBD | BG5NUC-LC144 |
| 3261 | 2P | BG5NUC-LC145 |
| 3262 | 2P | BG5NUC-LC146 |
| 3263 | 2P | BG5NUC-LC147 |
| 3264 | 2P | BG5NUC-LC148 |
| 3265 | 2P | BG5NUC-LC149 |
| 3266 | 2P | BG5NUC-LC150 |
| 3267 | 2P | BG5NUC-LC151 |
| 3268 | 2P | BG5NUC-LC152 |
| 3269 | 2P | BG5NUC-LC153 |
| 3270 | 2P | BG5NUC-LC154 |
| 3271 | 2P | BG5NUC-LC155 |
| 3272 | RBD | BG5NUC-LC156 |
| 3273 | RBD | BG5NUC-LC157 |
| 3274 | 2P | BG5NUC-LC158 |
| 3275 | 2P | BG5NUC-LC159 |
| 3276 | 2P | BG5NUC-LC160 |
| 3277 | 2P | BG5NUC-LC161 |
| 3278 | 2P | BG5NUC-LC162 |
| 3279 | RBD | BG5NUC-LC163 |
| 3280 | 2P | BG5NUC-LC164 |
| 3281 | 2P | BG5NUC-LC165 |
| 3282 | RBD | BG5NUC-LC166 |
| 3283 | 2P | BG5NUC-LC167 |
| 3284 | 2P | BG5NUC-LC168 |
| 3285 | 2P | BG5NUC-LC169 |
| 3286 | RBD | BG5NUC-LC170 |
| 3287 | RBD | BG5NUC-LC171 |
| 3288 | RBD | BG5NUC-LC172 |
| 3289 | 2P | BG5NUC-LC173 |
| 3290 | 2P | BG5NUC-LC174 |
| 3291 | 2P | BG5NUC-LC175 |
| 3292 | 2P | BG5NUC-LC176 |
| 3293 | 2P | BG5NUC-LC177 |
| 3294 | 2P | BG5NUC-LC178 |
| 3295 | RBD | BG5NUC-LC179 |
| 3296 | RBD | BG5NUC-LC180 |
| 3297 | 2P | BG5NUC-LC181 |
| 3298 | 2P | BG5NUC-LC182 |
| 3299 | 2P | BG5NUC-LC183 |
| 3300 | 2P | BG5NUC-LC184 |
| 3301 | 2P | BG5NUC-LC185 |
| 3302 | 2P | BG5NUC-LC186 |
| 3303 | 2P | BG5NUC-LC187 |
| 3304 | 2P | BG5NUC-LC188 |
| 3305 | 2P | BG5NUC-LC189 |
| 3306 | 2P | BG5NUC-LC190 |
| 3307 | 2P | BG5NUC-LC191 |
| 3308 | 2P | BG5NUC-LC192 |
| 3309 | 2P | BG5NUC-LC193 |
| 3310 | 2P | BG5NUC-LC194 |
| 3311 | 2P | BG5NUC-LC195 |
| 3312 | 2P | BG5NUC-LC196 |
| 3313 | RBD | BG5NUC-LC197 |
| 3314 | 2P | BG5NUC-LC198 |
| 3315 | 2P | BG5NUC-LC199 |
| 3316 | RBD | BG5NUC-LC200 |
| 3317 | 2P | BG5NUC-LC201 |
| 3318 | RBD | BG5NUC-LC202 |
| 3319 | 2P | BG5NUC-LC203 |
| 3320 | 2P | BG5NUC-LC204 |
| 3321 | 2P | BG5NUC-LC205 |
| 3322 | RBD | BG5NUC-LC206 |
| 3323 | 2P | BG5NUC-LC207 |
| 3324 | RBD | BG5NUC-LC208 |
| 3325 | 2P | BG5NUC-LC209 |
| 3326 | 2P | BG5NUC-LC210 |
| 3327 | RBD | BG5NUC-LC211 |
| 3328 | 2P | BG5NUC-LC212 |
| 3329 | 2P | BG5NUC-LC213 |
| 3330 | 2P | BG5NUC-LC214 |
| 3331 | 2P | BG5NUC-LC215 |
| 3332 | RBD | BG5NUC-LC216 |
| 3333 | RBD | BG5NUC-LC217 |
| 3334 | 2P | BG5NUC-LC218 |
| 3335 | RBD | BG5NUC-LC219 |
| 3336 | 2P | BG5NUC-LC220 |
| 3337 | 2P | BG5NUC-LC221 |
| 3338 | 2P | BG5NUC-LC222 |
| 3339 | RBD | BG5NUC-LC223 |
| 3340 | 2P | BG5NUC-LC224 |
| 3341 | 2P | BG5NUC-LC225 |
| 3342 | 2P | BG5NUC-LC226 |
| 3343 | RBD | BG5NUC-LC227 |
| 3344 | 2P | BG5NUC-LC228 |
| 3345 | 2P | BG5NUC-LC229 |
| 3346 | RBD | BG5NUC-LC230 |
| 3347 | RBD | BG5NUC-LC231 |
| 3348 | 2P | BG5NUC-LC232 |
| 3349 | 2P | BG5NUC-LC233 |
| 3350 | 2P | BG5NUC-LC234 |
| 3351 | 2P | BG5NUC-LC235 |
| 3352 | 2P | BG5NUC-LC236 |
| 3353 | 2P | BG5NUC-LC237 |
| 3354 | 2P | BG5NUC-LC238 |
| 3355 | 2P | BG5NUC-LC239 |
| 3356 | RBD | BG5NUC-LC240 |
| 3357 | RBD | BG5NUC-LC241 |
| 3358 | RBD | BG5NUC-LC242 |
| 3359 | RBD | BG5NUC-LC243 |
| 3360 | 2P | BG5NUC-LC244 |
| 3361 | 2P | BG5NUC-LC245 |
| 3362 | 2P | BG5NUC-LC246 |
| 3363 | RBD | BG5NUC-LC247 |
| 3364 | RBD | BG5NUC-LC248 |
| 3365 | RBD | BG5NUC-LC249 |
| 3366 | 2P | BG5NUC-LC250 |
| 3367 | 2P | BG5NUC-LC251 |
| 3368 | 2P | BG5NUC-LC252 |
| 3369 | 2P | BG5NUC-LC253 |
| 3370 | 2P | BG5NUC-LC254 |
| 3371 | 2P | BG5NUC-LC255 |
| 3372 | RBD | BG5NUC-LC256 |
| 3373 | 2P | BG5NUC-LC257 |
| 3374 | 2P | BG5NUC-LC258 |
| 3375 | 2P | BG5NUC-LC259 |
| 3376 | 2P | BG5NUC-LC260 |
| 3377 | 2P | BG5NUC-LC261 |
| 3378 | 2P | BG5NUC-LC262 |
| 3379 | 2P | BG5NUC-LC263 |
| 3380 | 2P | BG5NUC-LC264 |
| 3381 | 2P | BG5NUC-LC265 |
| 3382 | 2P | BG5NUC-LC266 |
| 3383 | RBD | BG5NUC-LC267 |
| 3384 | 2P | BG5NUC-LC268 |
| 3385 | RBD | BG5NUC-LC269 |
| 3386 | RBD | BG5NUC-LC270 |
| 3387 | 2P | BG5NUC-LC271 |
| 3388 | 2P | BG5NUC-LC272 |
| 3389 | RBD | BG5NUC-LC273 |
| 3390 | 2P | BG5NUC-LC274 |
| 3391 | 2P | BG5NUC-LC275 |
| 3392 | 2P | BG5NUC-LC276 |
| 3393 | RBD | BG5NUC-LC277 |
| 3394 | 2P | BG5NUC-LC278 |
| 3395 | RBD | BG5NUC-LC279 |
| 3396 | RBD | BG5NUC-LC280 |
| 3397 | 2P | BG5NUC-LC281 |
| 3398 | 2P | BG5NUC-LC282 |
| 3399 | RBD | BG5NUC-LC283 |
| 3400 | RBD | BG5NUC-LC284 |
| 3401 | RBD | BG5NUC-LC285 |
| 3402 | 2P | BG5NUC-LC286 |
| 3403 | 2P | BG5NUC-LC287 |
| 3404 | 2P | BG5NUC-LC288 |
| 3405 | 2P | BG5NUC-LC289 |
| 3406 | 2P | BG5NUC-LC290 |
| 3407 | 2P | BG5NUC-LC291 |
| 3408 | 2P | BG5NUC-LC292 |
| 3409 | 2P | BG5NUC-LC293 |
| 3410 | 2P | BG5NUC-LC294 |
| 3411 | 2P | BG5NUC-LC295 |
| 3412 | RBD | BG5NUC-LC296 |
| 3413 | RBD | BG5NUC-LC297 |
| 3414 | 2P | BG5NUC-LC298 |
| 3415 | RBD | BG5NUC-LC299 |
| 3416 | RBD | BG5NUC-LC300 |
| 3417 | 2P | BG5NUC-LC301 |
| 3418 | 2P | BG5NUC-LC302 |
| 3419 | RBD | BG5NUC-LC303 |
| 3420 | 2P | BG5NUC-LC304 |
| 3421 | RBD | BG5NUC-LC305 |
| 3422 | RBD | BG5NUC-LC306 |
| 3423 | RBD | BG5NUC-LC307 |
| 3424 | 2P | BG5NUC-LC308 |
| 3425 | 2P | BG5NUC-LC309 |
| 3426 | RBD | BG5NUC-LC310 |
| 3427 | 2P | BG5NUC-LC311 |
| 3428 | 2P | BG5NUC-LC312 |
| 3429 | 2P | BG5NUC-LC313 |
| 3430 | 2P | BG5NUC-LC314 |
| 3431 | RBD | BG5NUC-LC315 |
| 3432 | 2P | BG5NUC-LC316 |
| 3433 | RBD | BG5NUC-LC317 |
| 3434 | RBD | BG5NUC-LC318 |
| 3435 | 2P | BG5NUC-LC319 |
| 3436 | RBD | BG5NUC-LC320 |
| 3437 | RBD | BG5NUC-LC321 |
| 3438 | RBD | BG5NUC-LC322 |
| 3439 | 2P | BG5NUC-LC323 |
| 3440 | 2P | BG5NUC-LC324 |
| 3441 | 2P | BG5NUC-LC325 |
| 3442 | RBD | BG5NUC-LC326 |
| 3443 | 2P | BG5NUC-LC327 |
| 3444 | 2P | BG5NUC-LC328 |
| 3445 | 2P | BG5NUC-LC329 |
| 3446 | 2P | BG5NUC-LC330 |
| 3447 | 2P | BG5NUC-LC331 |
| 3448 | RBD | BG5NUC-LC332 |
| 3449 | RBD | BG5NUC-LC333 |
| 3450 | RBD | BG5NUC-LC334 |
| 3451 | RBD | BG5NUC-LC335 |
| 3452 | RBD | BG5NUC-LC336 |
| 3453 | RBD | BG5NUC-LC337 |
| 3454 | 2P | BG5NUC-LC338 |
| 3455 | 2P | BG5NUC-LC339 |
| 3456 | 2P | BG5NUC-LC340 |
| 3457 | 2P | BG5NUC-LC341 |
| 3458 | 2P | BG5NUC-LC342 |
| 3459 | 2P | BG5NUC-LC343 |
| 3460 | 2P | BG5NUC-LC344 |
| 3461 | 2P | BG5NUC-LC345 |
| 3462 | RBD | BG5NUC-LC346 |
| 3463 | RBD | BG5NUC-LC347 |
| 3464 | 2P | BG5NUC-LC348 |
| 3465 | 2P | BG5NUC-LC349 |
| 3466 | 2P | BG5NUC-LC350 |
| 3467 | 2P | BG5NUC-LC351 |
| 3468 | RBD | BG5NUC-LC352 |
| 3469 | 2P | BG5NUC-LC353 |
| 3470 | RBD | BG5NUC-LC354 |
| 3471 | RBD | BG5NUC-LC355 |
| 3472 | 2P | BG5NUC-LC356 |
| 3473 | RBD | BG5NUC-LC357 |
| 3474 | 2P | BG5NUC-LC358 |
| 3475 | RBD | BG5NUC-LC359 |
| 3476 | 2P | BG5NUC-LC360 |
| 3477 | 2P | BG5NUC-LC361 |
| 3478 | 2P | BG5NUC-LC362 |
| 3479 | RBD | BG5NUC-LC363 |
| 3480 | 2P | BG5NUC-LC364 |
| 3481 | 2P | BG5NUC-LC365 |
| 3482 | 2P | BG5NUC-LC366 |
| 3483 | 2P | BG5NUC-LC367 |
| 3484 | RBD | BG5NUC-LC368 |
| 3485 | RBD | BG5NUC-LC369 |
| 3486 | 2P | BG5NUC-LC370 |
| 3487 | RBD | BG5NUC-LC371 |
| 3488 | 2P | BG5NUC-LC372 |
| 3489 | RBD | BG5NUC-LC373 |
| 3490 | 2P | BG5NUC-LC374 |
| 3491 | 2P | BG5NUC-LC375 |
| 3492 | 2P | BG5NUC-LC376 |
| 3493 | RBD | BG5NUC-LC377 |
| 3494 | 2P | BG5NUC-LC378 |
| 3495 | 2P | BG5NUC-LC379 |
| 3496 | RBD | BG5NUC-LC380 |
| 3497 | RBD | BG5NUC-LC381 |
| 3498 | RBD | BG5NUC-LC382 |
| 3499 | RBD | BG5NUC-LC383 |
| 3500 | 2P | BG5NUC-LC384 |
| 3501 | 2P | BG5NUC-LC385 |
| 3502 | 2P | BG5NUC-LC386 |
| 3503 | 2P | BG5NUC-LC387 |
| 3504 | 2P | BG5NUC-LC388 |
| 3505 | 2P | BG5NUC-LC389 |
| 3506 | 2P | BG5NUC-LC390 |
| 3507 | 2P | BG5NUC-LC391 |
| 3508 | 2P | BG5NUC-LC392 |
| 3509 | 2P | BG5NUC-LC393 |
| 3510 | RBD | BG5NUC-LC394 |
| 3511 | RBD | BG5NUC-LC395 |
| 3512 | RBD | BG5NUC-LC396 |
| 3513 | 2P | BG5NUC-LC397 |
| 3514 | 2P | BG5NUC-LC398 |
| 3515 | 2P | BG5NUC-LC399 |
| 3516 | 2P | BG5NUC-LC400 |
| 3517 | 2P | BG5NUC-LC401 |
| 3518 | 2P | BG5NUC-LC402 |
| 3519 | 2P | BG5NUC-LC403 |
| 3520 | 2P | BG5NUC-LC404 |
| 3521 | 2P | BG5NUC-LC405 |
| 3522 | 2P | BG5NUC-LC406 |
| 3523 | RBD | BG5NUC-LC407 |
| 3524 | 2P | BG5NUC-LC408 |
| 3525 | RBD | BG5NUC-LC409 |
| 3526 | RBD | BG5NUC-LC410 |
| 3527 | 2P | BG5NUC-LC411 |
| 3528 | 2P | BG5NUC-LC412 |
| 3529 | 2P | BG5NUC-LC413 |
| 3530 | 2P | BG5NUC-LC414 |
| 3531 | 2P | BG5NUC-LC415 |
| 3532 | RBD | BG5NUC-LC416 |
| 3533 | 2P | BG5NUC-LC417 |
| 3534 | RBD | BG5NUC-LC418 |
| 3535 | 2P | BG5NUC-LC419 |
| 3536 | 2P | BG5NUC-LC420 |
| 3537 | 2P | BG5NUC-LC421 |
| 3538 | 2P | BG5NUC-LC422 |
| 3539 | 2P | BG5NUC-LC423 |
| 3540 | 2P | BG5NUC-LC424 |
| 3541 | RBD | BG5NUC-LC425 |
| 3542 | 2P | BG5NUC-LC426 |
| 3543 | RBD | BG5NUC-LC427 |
| 3544 | 2P | BG5NUC-LC428 |
| 3545 | 2P | BG5NUC-LC429 |
| 3546 | 2P | BG5NUC-LC430 |
| 3547 | 2P | BG5NUC-LC431 |
| 3548 | 2P | BG5NUC-LC432 |
| 3549 | 2P | BG5NUC-LC433 |
| 3550 | RBD | BG5NUC-LC434 |
| 3551 | RBD | BG5NUC-LC435 |
| 3552 | 2P | BG5NUC-LC436 |
| 3553 | RBD | BG5NUC-LC437 |
| 3554 | 2P | BG5NUC-LC438 |
| 3555 | 2P | BG5NUC-LC439 |
| 3556 | 2P | BG5NUC-LC440 |
| 3557 | 2P | BG5NUC-LC441 |
| 3558 | RBD | BG5NUC-LC442 |
| 3559 | 2P | BG5NUC-LC443 |
| 3560 | 2P | BG5NUC-LC444 |
| 3561 | 2P | BG5NUC-LC445 |
| 3562 | 2P | BG5NUC-LC446 |
| 3563 | 2P | BG5NUC-LC447 |
| 3564 | RBD | BG5NUC-LC448 |
| 3565 | 2P | BG5NUC-LC449 |
| 3566 | 2P | BG5NUC-LC450 |
| 3567 | 2P | BG5NUC-LC451 |
| 3568 | 2P | BG5NUC-LC452 |
| 3569 | 2P | BG5NUC-LC453 |
| 3570 | 2P | BG5NUC-LC454 |
| 3571 | 2P | BG5NUC-LC455 |
| 3572 | 2P | BG5NUC-LC456 |
| 3573 | 2P | BG5NUC-LC457 |
| 3574 | 2P | BG5NUC-LC458 |
| 3575 | 2P | BG5NUC-LC459 |
| 3576 | 2P | BG5NUC-LC460 |
| 3577 | RBD | BG5NUC-LC461 |
| 3578 | 2P | BG5NUC-LC462 |
| 3579 | 2P | BG5NUC-LC463 |
| 3580 | 2P | BG5NUC-LC464 |
| 3581 | 2P | BG5NUC-LC465 |
| 3582 | RBD | BG5NUC-LC466 |
| 3583 | 2P | BG5NUC-LC467 |
| 3584 | 2P | BG5NUC-LC468 |
| 3585 | 2P | BG5NUC-LC469 |
| 3586 | RBD | BG5NUC-LC470 |
| 3587 | RBD | BG5NUC-LC471 |
| 3588 | 2P | BG5NUC-LC472 |
| 3589 | RBD | BG5NUC-LC473 |
| 3590 | RBD | BG5NUC-LC474 |
| 3591 | RBD | BG5NUC-LC475 |
| 3592 | 2P | BG5NUC-LC476 |
| 3593 | RBD | BG6NUC-HC1 |
| 3594 | 2P | BG6NUC-HC2 |
| 3595 | RBD | BG6NUC-HC3 |
| 3596 | RBD | BG6NUC-HC4 |
| 3597 | RBD | BG6NUC-HC5 |
| 3598 | RBD | BG6NUC-HC6 |
| 3599 | 2P | BG6NUC-HC7 |
| 3600 | 2P | BG6NUC-HC8 |
| 3601 | RBD | BG6NUC-HC9 |
| 3602 | RBD | BG6NUC-HC10 |
| 3603 | RBD | BG6NUC-HC11 |
| 3604 | 2P | BG6NUC-HC12 |
| 3605 | 2P | BG6NUC-HC13 |
| 3606 | 2P | BG6NUC-HC14 |
| 3607 | 2P | BG6NUC-HC15 |
| 3608 | 2P | BG6NUC-HC16 |
| 3609 | 2P | BG6NUC-HC17 |
| 3610 | 2P | BG6NUC-HC18 |
| 3611 | 2P | BG6NUC-HC19 |
| 3612 | 2P | BG6NUC-HC20 |
| 3613 | 2P | BG6NUC-HC21 |
| 3614 | 2P | BG6NUC-HC22 |
| 3615 | 2P | BG6NUC-HC23 |
| 3616 | 2P | BG6NUC-HC24 |
| 3617 | RBD | BG6NUC-HC25 |
| 3618 | 2P | BG6NUC-HC26 |
| 3619 | 2F | BG6NUC-HC27 |
| 3620 | 2P | BG6NUC-HC28 |
| 3621 | 2F | BG6NUC-HC29 |
| 3622 | RBD | BG6NUC-HC30 |
| 3623 | RBD | BG6NUC-HC31 |
| 3624 | RBD | BG6NUC-HC32 |
| 3625 | RBD | BG6NUC-HC33 |
| 3626 | 2P | BG6NUC-HC34 |
| 3627 | RBD | BG6NUC-HC35 |
| 3628 | RBD | BG6NUC-HC36 |
| 3629 | 2P | BG6NUC-HC37 |
| 3630 | 2P | BG6NUC-HC38 |
| 3631 | 2P | BG6NUC-HC39 |
| 3632 | RBD | BG6NUC-HC40 |
| 3633 | 2P | BG6NUC-HC41 |
| 3634 | RBD | BG6NUC-HC42 |
| 3635 | 2P | BG6NUC-HC43 |
| 3636 | 2P | BG6NUC-HC44 |
| 3637 | RBD | BG6NUC-HC45 |
| 3638 | RBD | BG6NUC-HC46 |
| 3639 | 2P | BG6NUC-HC47 |
| 3640 | 2F | BG6NUC-HC48 |
| 3641 | RBD | BG6NUC-HC49 |
| 3642 | RBD | BG6NUC-HC50 |
| 3643 | 2P | BG6NUC-HC51 |
| 3644 | 2P | BG6NUC-HC52 |
| 3645 | RBD | BG6NUC-HC53 |
| 3646 | 2P | BG6NUC-HC54 |
| 3647 | RBD | BG6NUC-HC55 |
| 3648 | 2P | BG6NUC-HC56 |
| 3649 | 2P | BG6NUC-HC57 |
| 3650 | 2P | BG6NUC-HC58 |
| 3651 | 2P | BG6NUC-HC59 |
| 3652 | 2P | BG6NUC-HC60 |
| 3653 | RBD | BG6NUC-HC61 |
| 3654 | 2P | BG6NUC-HC62 |
| 3655 | RBD | BG6NUC-HC63 |
| 3656 | 2P | BG6NUC-HC64 |
| 3657 | 2P | BG6NUC-HC65 |
| 3658 | RBD | BG6NUC-HC66 |
| 3659 | 2P | BG6NUC-HC67 |
| 3660 | 2P | BG6NUC-HC68 |
| 3661 | RBD | BG6NUC-HC69 |
| 3662 | 2P | BG6NUC-HC70 |
| 3663 | 2F | BG6NUC-HC71 |
| 3664 | 2P | BG6NUC-HC72 |
| 3665 | RBD | BG6NUC-HC73 |
| 3666 | RBD | BG6NUC-HC74 |
| 3667 | RBD | BG6NUC-HC75 |
| 3668 | RBD | BG6NUC-HC76 |
| 3669 | RBD | BG6NUC-HC77 |
| 3670 | RBD | BG6NUC-HC78 |
| 3671 | 2P | BG6NUC-HC79 |
| 3672 | 2P | BG6NUC-HC80 |
| 3673 | 2P | BG6NUC-HC81 |
| 3674 | 2P | BG6NUC-HC82 |
| 3675 | 2P | BG6NUC-HC83 |
| 3676 | RBD | BG6NUC-HC84 |
| 3677 | 2P | BG6NUC-HC85 |
| 3678 | RBD | BG6NUC-HC86 |
| 3679 | 2P | BG6NUC-HC87 |
| 3680 | 2P | BG6NUC-HC88 |
| 3681 | RBD | BG6NUC-HC89 |
| 3682 | 2P | BG6NUC-HC90 |
| 3683 | 2P | BG6NUC-HC91 |
| 3684 | RBD | BG6NUC-HC92 |
| 3685 | RBD | BG6NUC-HC93 |
| 3686 | 2P | BG6NUC-HC94 |
| 3687 | 2P | BG6NUC-HC95 |
| 3688 | 2P | BG6NUC-HC96 |
| 3689 | 2P | BG6NUC-HC97 |
| 3690 | RBD | BG6NUC-HC98 |
| 3691 | 2P | BG6NUC-HC99 |
| 3692 | RBD | BG6NUC-HC100 |
| 3693 | 2P | BG6NUC-HC101 |
| 3694 | 2P | BG6NUC-HC102 |
| 3695 | 2P | BG6NUC-HC103 |
| 3696 | 2P | BG6NUC-HC104 |
| 3697 | 2P | BG6NUC-HC105 |
| 3698 | RBD | BG6NUC-HC106 |
| 3699 | RBD | BG6NUC-HC107 |
| 3700 | RBD | BG6NUC-HC108 |
| 3701 | RBD | BG6NUC-HC109 |
| 3702 | 2P | BG6NUC-HC110 |
| 3703 | 2P | BG6NUC-HC111 |
| 3704 | RBD | BG6NUC-HC112 |
| 3705 | 2P | BG6NUC-HC113 |
| 3706 | RBD | BG6NUC-HC114 |
| 3707 | RBD | BG6NUC-HC115 |
| 3708 | RBD | BG6NUC-HC116 |
| 3709 | RBD | BG6NUC-HC117 |
| 3710 | 2P | BG6NUC-HC118 |
| 3711 | RBD | BG6NUC-HC119 |
| 3712 | 2P | BG6NUC-HC120 |
| 3713 | 2P | BG6NUC-HC121 |
| 3714 | 2P | BG6NUC-HC122 |
| 3715 | 2P | BG6NUC-HC123 |
| 3716 | RBD | BG6NUC-HC124 |
| 3717 | RBD | BG6NUC-HC125 |
| 3718 | 2P | BG6NUC-HC126 |
| 3719 | RBD | BG6NUC-HC127 |
| 3720 | 2P | BG6NUC-HC128 |
| 3721 | 2P | BG6NUC-HC129 |
| 3722 | 2P | BG6NUC-HC130 |
| 3723 | 2P | BG6NUC-HC131 |
| 3724 | RBD | BG6NUC-HC132 |
| 3725 | 2P | BG6NUC-HC133 |
| 3726 | 2P | BG6NUC-HC134 |
| 3727 | RBD | BG6NUC-HC135 |
| 3728 | RBD | BG6NUC-HC136 |
| 3729 | RBD | BG6NUC-HC137 |
| 3730 | 2P | BG6NUC-HC138 |
| 3731 | 2P | BG6NUC-HC139 |
| 3732 | 2P | BG6NUC-HC140 |
| 3733 | 2P | BG6NUC-HC141 |
| 3734 | RBD | BG6NUC-HC142 |
| 3735 | 2P | BG6NUC-HC143 |
| 3736 | 2P | BG6NUC-HC144 |
| 3737 | 2P | BG6NUC-HC145 |
| 3738 | RBD | BG6NUC-HC146 |
| 3739 | RBD | BG6NUC-HC147 |
| 3740 | 2P | BG6NUC-HC148 |
| 3741 | 2P | BG6NUC-HC149 |
| 3742 | RBD | BG6NUC-HC150 |
| 3743 | RBD | BG6NUC-HC151 |
| 3744 | 2P | BG6NUC-HC152 |
| 3745 | 2P | BG6NUC-HC153 |
| 3746 | RBD | BG6NUC-HC154 |
| 3747 | RBD | BG6NUC-HC155 |
| 3748 | RBD | BG6NUC-HC156 |
| 3749 | 2P | BG6NUC-HC157 |
| 3750 | RBD | BG6NUC-HC158 |
| 3751 | 2P | BG6NUC-HC159 |
| 3752 | 2P | BG6NUC-HC160 |
| 3753 | 2P | BG6NUC-HC161 |
| 3754 | 2P | BG6NUC-HC162 |
| 3755 | 2P | BG6NUC-HC163 |
| 3756 | RBD | BG6NUC-HC164 |
| 3757 | 2P | BG6NUC-HC165 |
| 3758 | RBD | BG6NUC-HC166 |
| 3759 | RBD | BG6NUC-HC167 |
| 3760 | 2P | BG6NUC-HC168 |
| 3761 | RBD | BG6NUC-HC169 |
| 3762 | 2P | BG6NUC-HC170 |
| 3763 | 2P | BG6NUC-HC171 |
| 3764 | 2P | BG6NUC-HC172 |
| 3765 | 2P | BG6NUC-HC173 |
| 3766 | RBD | BG6NUC-HC174 |
| 3767 | 2P | BG6NUC-HC175 |
| 3768 | RBD | BG6NUC-HC176 |
| 3769 | RBD | BG6NUC-HC177 |
| 3770 | 2P | BG6NUC-HC178 |
| 3771 | 2P | BG6NUC-HC179 |
| 3772 | 2P | BG6NUC-HC180 |
| 3773 | RBD | BG6NUC-HC181 |
| 3774 | RBD | BG6NUC-HC182 |
| 3775 | 2P | BG6NUC-HC183 |
| 3776 | 2F | BG6NUC-HC184 |
| 3777 | 2P | BG6NUC-HC185 |
| 3778 | 2P | BG6NUC-HC186 |
| 3779 | RBD | BG6NUC-HC187 |
| 3780 | 2P | BG6NUC-HC188 |
| 3781 | RBD | BG6NUC-HC189 |
| 3782 | 2P | BG6NUC-HC190 |
| 3783 | RBD | BG6NUC-HC191 |
| 3784 | RBD | BG6NUC-HC192 |
| 3785 | 2P | BG6NUC-HC193 |
| 3786 | RBD | BG6NUC-HC194 |
| 3787 | 2P | BG6NUC-HC195 |
| 3788 | 2P | BG6NUC-HC196 |
| 3789 | 2P | BG6NUC-HC197 |
| 3790 | 2P | BG6NUC-HC198 |
| 3791 | 2F | BG6NUC-HC199 |
| 3792 | 2P | BG6NUC-HC200 |
| 3793 | RBD | BG6NUC-HC201 |
| 3794 | RBD | BG6NUC-HC202 |
| 3795 | 2P | BG6NUC-HC203 |
| 3796 | RBD | BG6NUC-HC204 |
| 3797 | RBD | BG6NUC-HC205 |
| 3798 | 2P | BG6NUC-HC206 |
| 3799 | 2P | BG6NUC-HC207 |
| 3800 | 2P | BG6NUC-HC208 |
| 3801 | RBD | BG6NUC-HC209 |
| 3802 | 2P | BG6NUC-HC210 |
| 3803 | RBD | BG6NUC-HC211 |
| 3804 | 2P | BG6NUC-HC212 |
| 3805 | 2P | BG6NUC-HC213 |
| 3806 | 2P | BG6NUC-HC214 |
| 3807 | 2P | BG6NUC-HC215 |
| 3808 | 2P | BG6NUC-HC216 |
| 3809 | 2P | BG6NUC-HC217 |
| 3810 | 2P | BG6NUC-HC218 |
| 3811 | RBD | BG6NUC-HC219 |
| 3812 | RBD | BG6NUC-HC220 |
| 3813 | 2P | BG6NUC-HC221 |
| 3814 | 2P | BG6NUC-HC222 |
| 3815 | RBD | BG6NUC-HC223 |
| 3816 | RBD | BG6NUC-HC224 |
| 3817 | RBD | BG6NUC-HC225 |
| 3818 | 2P | BG6NUC-HC226 |
| 3819 | 2P | BG6NUC-HC227 |
| 3820 | RBD | BG6NUC-HC228 |
| 3821 | RBD | BG6NUC-HC229 |
| 3822 | RBD | BG6NUC-HC230 |
| 3823 | RBD | BG6NUC-HC231 |
| 3824 | 2P | BG6NUC-HC232 |
| 3825 | RBD | BG6NUC-HC233 |
| 3826 | 2P | BG6NUC-HC234 |
| 3827 | RBD | BG6NUC-HC235 |
| 3828 | RBD | BG6NUC-HC236 |
| 3829 | RBD | BG6NUC-HC237 |
| 3830 | 2P | BG6NUC-HC238 |
| 3831 | 2P | BG6NUC-HC239 |
| 3832 | RBD | BG6NUC-HC240 |
| 3833 | 2P | BG6NUC-HC241 |
| 3834 | 2P | BG6NUC-HC242 |
| 3835 | RBD | BG6NUC-HC243 |
| 3836 | 2P | BG6NUC-HC244 |
| 3837 | 2P | BG6NUC-HC245 |
| 3838 | 2P | BG6NUC-HC246 |
| 3839 | RBD | BG6NUC-LC1 |
| 3840 | 2P | BG6NUC-LC2 |
| 3841 | RBD | BG6NUC-LC3 |
| 3842 | RBD | BG6NUC-LC4 |
| 3843 | RBD | BG6NUC-LC5 |
| 3844 | RBD | BG6NUC-LC6 |
| 3845 | 2P | BG6NUC-LC7 |
| 3846 | 2P | BG6NUC-LC8 |
| 3847 | RBD | BG6NUC-LC9 |
| 3848 | RBD | BG6NUC-LC10 |
| 3849 | RBD | BG6NUC-LC11 |
| 3850 | 2P | BG6NUC-LC12 |
| 3851 | 2P | BG6NUC-LC13 |
| 3852 | 2P | BG6NUC-LC14 |
| 3853 | 2P | BG6NUC-LC15 |
| 3854 | 2P | BG6NUC-LC16 |
| 3855 | 2P | BG6NUC-LC17 |
| 3856 | 2P | BG6NUC-LC18 |
| 3857 | 2P | BG6NUC-LC19 |
| 3858 | 2P | BG6NUC-LC20 |
| 3859 | 2P | BG6NUC-LC21 |
| 3860 | 2P | BG6NUC-LC22 |
| 3861 | 2P | BG6NUC-LC23 |
| 3862 | 2P | BG6NUC-LC24 |
| 3863 | RBD | BG6NUC-LC25 |
| 3864 | 2P | BG6NUC-LC26 |
| 3865 | 2P | BG6NUC-LC27 |
| 3866 | 2P | BG6NUC-LC28 |
| 3867 | 2P | BG6NUC-LC29 |
| 3868 | RBD | BG6NUC-LC30 |
| 3869 | RBD | BG6NUC-LC31 |
| 3870 | RBD | BG6NUC-LC32 |
| 3871 | RBD | BG6NUC-LC33 |
| 3872 | 2P | BG6NUC-LC34 |
| 3873 | RBD | BG6NUC-LC35 |
| 3874 | RBD | BG6NUC-LC36 |
| 3875 | 2P | BG6NUC-LC37 |
| 3876 | 2P | BG6NUC-LC38 |
| 3877 | 2P | BG6NUC-LC39 |
| 3878 | RBD | BG6NUC-LC40 |
| 3879 | 2P | BG6NUC-LC41 |
| 3880 | RBD | BG6NUC-LC42 |
| 3881 | 2P | BG6NUC-LC43 |
| 3882 | 2P | BG6NUC-LC44 |
| 3883 | RBD | BG6NUC-LC45 |
| 3884 | RBD | BG6NUC-LC46 |
| 3885 | 2P | BG6NUC-LC47 |
| 3886 | 2P | BG6NUC-LC48 |
| 3887 | RBD | BG6NUC-LC49 |
| 3888 | RBD | BG6NUC-LC50 |
| 3889 | 2P | BG6NUC-LC51 |
| 3890 | 2P | BG6NUC-LC52 |
| 3891 | RBD | BG6NUC-LC53 |
| 3892 | 2P | BG6NUC-LC54 |
| 3893 | RBD | BG6NUC-LC55 |
| 3894 | 2F | BG6NUC-LC56 |
| 3895 | 2F | BG6NUC-LC57 |
| 3896 | 2P | BG6NUC-LC58 |
| 3897 | 2P | BG6NUC-LC59 |
| 3898 | 2P | BG6NUC-LC60 |
| 3899 | RBD | BG6NUC-LC61 |
| 3900 | 2F | BG6NUC-LC62 |
| 3901 | RBD | BG6NUC-LC63 |
| 3902 | 2P | BG6NUC-LC64 |
| 3903 | 2P | BG6NUC-LC65 |
| 3904 | RBD | BG6NUC-LC66 |
| 3905 | 2P | BG6NUC-LC67 |
| 3906 | 2P | BG6NUC-LC68 |
| 3907 | RBD | BG6NUC-LC69 |
| 3908 | 2P | BG6NUC-LC70 |
| 3909 | 2P | BG6NUC-LC71 |
| 3910 | 2P | BG6NUC-LC72 |
| 3911 | RBD | BG6NUC-LC73 |
| 3912 | RBD | BG6NUC-LC74 |
| 3913 | RBD | BG6NUC-LC75 |
| 3914 | RBD | BG6NUC-LC76 |
| 3915 | RBD | BG6NUC-LC77 |
| 3916 | RBD | BG6NUC-LC78 |
| 3917 | 2P | BG6NUC-LC79 |
| 3918 | 2P | BG6NUC-LC80 |
| 3919 | 2P | BG6NUC-LC81 |
| 3920 | 2P | BG6NUC-LC82 |
| 3921 | 2P | BG6NUC-LC83 |
| 3922 | RBD | BG6NUC-LC84 |
| 3923 | 2P | BG6NUC-LC85 |
| 3924 | RBD | BG6NUC-LC86 |
| 3925 | 2P | BG6NUC-LC87 |
| 3926 | 2P | BG6NUC-LC88 |
| 3927 | RBD | BG6NUC-LC89 |
| 3928 | 2P | BG6NUC-LC90 |
| 3929 | 2P | BG6NUC-LC91 |
| 3930 | RBD | BG6NUC-LC92 |
| 3931 | RBD | BG6NUC-LC93 |
| 3932 | 2P | BG6NUC-LC94 |
| 3933 | 2P | BG6NUC-LC95 |
| 3934 | 2P | BG6NUC-LC96 |
| 3935 | 2P | BG6NUC-LC97 |
| 3936 | RBD | BG6NUC-LC98 |
| 3937 | 2P | BG6NUC-LC99 |
| 3938 | RBD | BG6NUC-LC100 |
| 3939 | 2P | BG6NUC-LC101 |
| 3940 | 2P | BG6NUC-LC102 |
| 3941 | 2P | BG6NUC-LC103 |
| 3942 | 2P | BG6NUC-LC104 |
| 3943 | 2P | BG6NUC-LC105 |
| 3944 | RBD | BG6NUC-LC106 |
| 3945 | RBD | BG6NUC-LC107 |
| 3946 | RBD | BG6NUC-LC108 |
| 3947 | RBD | BG6NUC-LC109 |
| 3948 | 2P | BG6NUC-LC110 |
| 3949 | 2P | BG6NUC-LC111 |
| 3950 | RBD | BG6NUC-LC112 |
| 3951 | 2F | BG6NUC-LC113 |
| 3952 | RBD | BG6NUC-LC114 |
| 3953 | RBD | BG6NUC-LC115 |
| 3954 | RBD | BG6NUC-LC116 |
| 3955 | RBD | BG6NUC-LC117 |
| 3956 | 2P | BG6NUC-LC118 |
| 3957 | RBD | BG6NUC-LC119 |
| 3958 | 2P | BG6NUC-LC120 |
| 3959 | 2P | BG6NUC-LC121 |
| 3960 | 2P | BG6NUC-LC122 |
| 3961 | 2P | BG6NUC-LC123 |
| 3962 | RBD | BG6NUC-LC124 |
| 3963 | RBD | BG6NUC-LC125 |
| 3964 | 2P | BG6NUC-LC126 |
| 3965 | RBD | BG6NUC-LC127 |
| 3966 | 2P | BG6NUC-LC128 |
| 3967 | 2P | BG6NUC-LC129 |
| 3968 | 2P | BG6NUC-LC130 |
| 3969 | 2P | BG6NUC-LC131 |
| 3970 | RBD | BG6NUC-LC132 |
| 3971 | 2P | BG6NUC-LC133 |
| 3972 | 2P | BG6NUC-LC134 |
| 3973 | RBD | BG6NUC-LC135 |
| 3974 | RBD | BG6NUC-LC136 |
| 3975 | RBD | BG6NUC-LC137 |
| 3976 | 2P | BG6NUC-LC138 |
| 3977 | 2P | BG6NUC-LC139 |
| 3978 | 2P | BG6NUC-LC140 |
| 3979 | 2P | BG6NUC-LC141 |
| 3980 | RBD | BG6NUC-LC142 |
| 3981 | 2P | BG6NUC-LC143 |
| 3982 | 2P | BG6NUC-LC144 |
| 3983 | 2P | BG6NUC-LC145 |
| 3984 | RBD | BG6NUC-LC146 |
| 3985 | RBD | BG6NUC-LC147 |
| 3986 | 2P | BG6NUC-LC148 |
| 3987 | 2P | BG6NUC-LC149 |
| 3988 | RBD | BG6NUC-LC150 |
| 3989 | RBD | BG6NUC-LC151 |
| 3990 | 2P | BG6NUC-LC152 |
| 3991 | 2P | BG6NUC-LC153 |
| 3992 | RBD | BG6NUC-LC154 |
| 3993 | RBD | BG6NUC-LC155 |
| 3994 | RBD | BG6NUC-LC156 |
| 3995 | 2P | BG6NUC-LC157 |
| 3996 | RBD | BG6NUC-LC158 |
| 3997 | 2P | BG6NUC-LC159 |
| 3998 | 2P | BG6NUC-LC160 |
| 3999 | 2P | BG6NUC-LC161 |
| 4000 | 2P | BG6NUC-LC162 |
| 4001 | 2P | BG6NUC-LC163 |
| 4002 | RBD | BG6NUC-LC164 |
| 4003 | 2P | BG6NUC-LC165 |
| 4004 | RBD | BG6NUC-LC166 |
| 4005 | RBD | BG6NUC-LC167 |
| 4006 | 2P | BG6NUC-LC168 |
| 4007 | RBD | BG6NUC-LC169 |
| 4008 | 2P | BG6NUC-LC170 |
| 4009 | 2P | BG6NUC-LC171 |
| 4010 | 2P | BG6NUC-LC172 |
| 4011 | 2P | BG6NUC-LC173 |
| 4012 | RBD | BG6NUC-LC174 |
| 4013 | 2P | BG6NUC-LC175 |
| 4014 | RBD | BG6NUC-LC176 |
| 4015 | RBD | BG6NUC-LC177 |
| 4016 | 2P | BG6NUC-LC178 |
| 4017 | 2P | BG6NUC-LC179 |
| 4018 | 2P | BG6NUC-LC180 |
| 4019 | RBD | BG6NUC-LC181 |
| 4020 | RBD | BG6NUC-LC182 |
| 4021 | 2P | BG6NUC-LC183 |
| 4022 | 2P | BG6NUC-LC184 |
| 4023 | 2P | BG6NUC-LC185 |
| 4024 | 2P | BG6NUC-LC186 |
| 4025 | RBD | BG6NUC-LC187 |
| 4026 | 2F | BG6NUC-LC188 |
| 4027 | RBD | BG6NUC-LC189 |
| 4028 | 2P | BG6NUC-LC190 |
| 4029 | RBD | BG6NUC-LC191 |
| 4030 | RBD | BG6NUC-LC192 |
| 4031 | 2P | BG6NUC-LC193 |
| 4032 | RBD | BG6NUC-LC194 |
| 4033 | 2P | BG6NUC-LC195 |
| 4034 | 2P | BG6NUC-LC196 |
| 4035 | 2P | BG6NUC-LC197 |
| 4036 | 2P | BG6NUC-LC198 |
| 4037 | 2P | BG6NUC-LC199 |
| 4038 | 2P | BG6NUC-LC200 |
| 4039 | RBD | BG6NUC-LC201 |
| 4040 | RBD | BG6NUC-LC202 |
| 4041 | 2P | BG6NUC-LC203 |
| 4042 | RBD | BG6NUC-LC204 |
| 4043 | RBD | BG6NUC-LC205 |
| 4044 | 2P | BG6NUC-LC206 |
| 4045 | 2P | BG6NUC-LC207 |
| 4046 | 2P | BG6NUC-LC208 |
| 4047 | RBD | BG6NUC-LC209 |
| 4048 | 2P | BG6NUC-LC210 |
| 4049 | RBD | BG6NUC-LC211 |
| 4050 | 2P | BG6NUC-LC212 |
| 4051 | 2P | BG6NUC-LC213 |
| 4052 | 2P | BG6NUC-LC214 |
| 4053 | 2P | BG6NUC-LC215 |
| 4054 | 2P | BG6NUC-LC216 |
| 4055 | 2P | BG6NUC-LC217 |
| 4056 | 2P | BG6NUC-LC218 |
| 4057 | RBD | BG6NUC-LC219 |
| 4058 | RBD | BG6NUC-LC220 |
| 4059 | 2P | BG6NUC-LC221 |
| 4060 | 2P | BG6NUC-LC222 |
| 4061 | RBD | BG6NUC-LC223 |
| 4062 | RBD | BG6NUC-LC224 |
| 4063 | RBD | BG6NUC-LC225 |
| 4064 | 2P | BG6NUC-LC226 |
| 4065 | 2P | BG6NUC-LC227 |
| 4066 | RBD | BG6NUC-LC228 |
| 4067 | RBD | BG6NUC-LC229 |
| 4068 | RBD | BG6NUC-LC230 |
| 4069 | RBD | BG6NUC-LC231 |
| 4070 | 2P | BG6NUC-LC232 |
| 4071 | RBD | BG6NUC-LC233 |
| 4072 | 2P | BG6NUC-LC234 |
| 4073 | RBD | BG6NUC-LC235 |
| 4074 | RBD | BG6NUC-LC236 |
| 4075 | RBD | BG6NUC-LC237 |
| 4076 | 2P | BG6NUC-LC238 |
| 4077 | 2P | BG6NUC-LC239 |
| 4078 | RBD | BG6NUC-LC240 |
| 4079 | 2P | BG6NUC-LC241 |
| 4080 | 2P | BG6NUC-LC242 |
| 4081 | RBD | BG6NUC-LC243 |
| 4082 | 2P | BG6NUC-LC244 |
| 4083 | 2P | BG6NUC-LC245 |
| 4084 | 2P | BG6NUC-LC246 |
| 4085 | 2P | BG7NUC-HC1 |
| 4086 | 2P | BG7NUC-HC2 |
| 4087 | 2P | BG7NUC-HC3 |
| 4088 | 2P | BG7NUC-HC4 |
| 4089 | 2P | BG7NUC-HC5 |
| 4090 | 2P | BG7NUC-HC6 |
| 4091 | 2P | BG7NUC-HC7 |
| 4092 | 2P | BG7NUC-HC8 |
| 4093 | 2P | BG7NUC-HC9 |
| 4094 | 2P | BG7NUC-HC10 |
| 4095 | 2P | BG7NUC-HC11 |
| 4096 | 2P | BG7NUC-HC12 |
| 4097 | 2P | BG7NUC-HC13 |
| 4098 | 2P | BG7NUC-HC14 |
| 4099 | 2P | BG7NUC-HC15 |
| 4100 | 2P | BG7NUC-HC16 |
| 4101 | 2P | BG7NUC-HC17 |
| 4102 | 2P | BG7NUC-HC18 |
| 4103 | 2P | BG7NUC-HC19 |
| 4104 | 2P | BG7NUC-HC20 |
| 4105 | 2P | BG7NUC-HC21 |
| 4106 | 2P | BG7NUC-HC22 |
| 4107 | 2P | BG7NUC-HC23 |
| 4108 | 2P | BG7NUC-HC24 |
| 4109 | 2P | BG7NUC-HC25 |
| 4110 | 2P | BG7NUC-HC26 |
| 4111 | 2P | BG7NUC-HC27 |
| 4112 | 2P | BG7NUC-HC28 |
| 4113 | 2P | BG7NUC-HC29 |
| 4114 | 2P | BG7NUC-HC30 |
| 4115 | 2P | BG7NUC-HC31 |
| 4116 | 2P | BG7NUC-HC32 |
| 4117 | RBD | BG7NUC-HC33 |
| 4118 | RBD | BG7NUC-HC34 |
| 4119 | RBD | BG7NUC-HC35 |
| 4120 | RBD | BG7NUC-HC36 |
| 4121 | 2P | BG7NUC-HC37 |
| 4122 | 2P | BG7NUC-HC38 |
| 4123 | 2P | BG7NUC-HC39 |
| 4124 | 2P | BG7NUC-HC40 |
| 4125 | 2P | BG7NUC-HC41 |
| 4126 | 2P | BG7NUC-HC42 |
| 4127 | 2P | BG7NUC-HC43 |
| 4128 | 2P | BG7NUC-HC44 |
| 4129 | 2P | BG7NUC-HC45 |
| 4130 | 2P | BG7NUC-HC46 |
| 4131 | 2P | BG7NUC-HC47 |
| 4132 | 2P | BG7NUC-HC48 |
| 4133 | 2P | BG7NUC-HC49 |
| 4134 | 2P | BG7NUC-HC50 |
| 4135 | 2P | BG7NUC-HC51 |
| 4136 | 2P | BG7NUC-HC52 |
| 4137 | 2P | BG7NUC-HC53 |
| 4138 | 2P | BG7NUC-HC54 |
| 4139 | 2P | BG7NUC-HC55 |
| 4140 | 2P | BG7NUC-HC56 |
| 4141 | 2P | BG7NUC-HC57 |
| 4142 | 2P | BG7NUC-HC58 |
| 4143 | 2P | BG7NUC-HC59 |
| 4144 | 2P | BG7NUC-HC60 |
| 4145 | RBD | BG7NUC-HC61 |
| 4146 | 2P | BG7NUC-HC62 |
| 4147 | 2P | BG7NUC-HC63 |
| 4148 | RBD | BG7NUC-HC64 |
| 4149 | RBD | BG7NUC-HC65 |
| 4150 | RBD | BG7NUC-HC66 |
| 4151 | RBD | BG7NUC-HC67 |
| 4152 | RBD | BG7NUC-HC68 |
| 4153 | 2P | BG7NUC-HC69 |
| 4154 | 2P | BG7NUC-HC70 |
| 4155 | 2P | BG7NUC-HC71 |
| 4156 | 2P | BG7NUC-HC72 |
| 4157 | 2P | BG7NUC-HC73 |
| 4158 | 2P | BG7NUC-HC74 |
| 4159 | 2P | BG7NUC-HC75 |
| 4160 | 2P | BG7NUC-HC76 |
| 4161 | 2P | BG7NUC-HC77 |
| 4162 | 2P | BG7NUC-HC78 |
| 4163 | 2P | BG7NUC-HC79 |
| 4164 | 2P | BG7NUC-HC80 |
| 4165 | 2P | BG7NUC-HC81 |
| 4166 | 2P | BG7NUC-HC82 |
| 4167 | 2P | BG7NUC-HC83 |
| 4168 | 2P | BG7NUC-HC84 |
| 4169 | 2P | BG7NUC-HC85 |
| 4170 | 2P | BG7NUC-HC86 |
| 4171 | 2P | BG7NUC-HC87 |
| 4172 | 2P | BG7NUC-HC88 |
| 4173 | 2P | BG7NUC-HC89 |
| 4174 | 2P | BG7NUC-HC90 |
| 4175 | 2P | BG7NUC-HC91 |
| 4176 | 2P | BG7NUC-HC92 |
| 4177 | 2P | BG7NUC-HC93 |
| 4178 | 2P | BG7NUC-HC94 |
| 4179 | 2P | BG7NUC-HC95 |
| 4180 | 2P | BG7NUC-HC96 |
| 4181 | 2P | BG7NUC-HC97 |
| 4182 | 2P | BG7NUC-HC98 |
| 4183 | 2P | BG7NUC-HC99 |
| 4184 | 2P | BG7NUC-HC100 |
| 4185 | 2P | BG7NUC-HC101 |
| 4186 | 2P | BG7NUC-HC102 |
| 4187 | 2P | BG7NUC-HC103 |
| 4188 | 2P | BG7NUC-HC104 |
| 4189 | 2P | BG7NUC-HC105 |
| 4190 | 2P | BG7NUC-HC106 |
| 4191 | 2P | BG7NUC-HC107 |
| 4192 | 2P | BG7NUC-HC108 |
| 4193 | 2P | BG7NUC-HC109 |
| 4194 | 2P | BG7NUC-HC110 |
| 4195 | 2P | BG7NUC-HC111 |
| 4196 | 2F | BG7NUC-HC112 |
| 4197 | 2P | BG7NUC-HC113 |
| 4198 | 2P | BG7NUC-HC114 |
| 4199 | 2P | BG7NUC-HC115 |
| 4200 | 2P | BG7NUC-HC116 |
| 4201 | 2P | BG7NUC-HC117 |
| 4202 | 2P | BG7NUC-HC118 |
| 4203 | 2P | BG7NUC-HC119 |
| 4204 | 2P | BG7NUC-HC120 |
| 4205 | 2P | BG7NUC-HC121 |
| 4206 | 2P | BG7NUC-HC122 |
| 4207 | 2P | BG7NUC-HC123 |
| 4208 | 2P | BG7NUC-HC124 |
| 4209 | 2P | BG7NUC-HC125 |
| 4210 | 2P | BG7NUC-HC126 |
| 4211 | 2P | BG7NUC-HC127 |
| 4212 | 2P | BG7NUC-HC128 |
| 4213 | 2P | BG7NUC-HC129 |
| 4214 | 2P | BG7NUC-HC130 |
| 4215 | 2P | BG7NUC-HC131 |
| 4216 | 2P | BG7NUC-HC132 |
| 4217 | 2P | BG7NUC-HC133 |
| 4218 | 2P | BG7NUC-HC134 |
| 4219 | 2P | BG7NUC-HC135 |
| 4220 | 2P | BG7NUC-HC136 |
| 4221 | 2P | BG7NUC-HC137 |
| 4222 | 2P | BG7NUC-HC138 |
| 4223 | 2P | BG7NUC-HC139 |
| 4224 | 2P | BG7NUC-HC140 |
| 4225 | 2P | BG7NUC-HC141 |
| 4226 | 2P | BG7NUC-HC142 |
| 4227 | 2P | BG7NUC-HC143 |
| 4228 | 2P | BG7NUC-HC144 |
| 4229 | 2P | BG7NUC-HC145 |
| 4230 | 2P | BG7NUC-HC146 |
| 4231 | 2P | BG7NUC-HC147 |
| 4232 | 2P | BG7NUC-HC148 |
| 4233 | 2P | BG7NUC-HC149 |
| 4234 | 2P | BG7NUC-HC150 |
| 4235 | 2P | BG7NUC-HC151 |
| 4236 | 2P | BG7NUC-HC152 |
| 4237 | 2P | BG7NUC-HC153 |
| 4238 | 2P | BG7NUC-HC154 |
| 4239 | 2P | BG7NUC-HC155 |
| 4240 | 2P | BG7NUC-HC156 |
| 4241 | 2P | BG7NUC-HC157 |
| 4242 | 2P | BG7NUC-HC158 |
| 4243 | 2P | BG7NUC-HC159 |
| 4244 | 2P | BG7NUC-HC160 |
| 4245 | 2P | BG7NUC-HC161 |
| 4246 | 2P | BG7NUC-HC162 |
| 4247 | 2P | BG7NUC-HC163 |
| 4248 | 2P | BG7NUC-HC164 |
| 4249 | 2P | BG7NUC-HC165 |
| 4250 | 2P | BG7NUC-HC166 |
| 4251 | 2P | BG7NUC-HC167 |
| 4252 | 2P | BG7NUC-HC168 |
| 4253 | 2P | BG7NUC-HC169 |
| 4254 | 2P | BG7NUC-HC170 |
| 4255 | 2P | BG7NUC-HC171 |
| 4256 | 2P | BG7NUC-HC172 |
| 4257 | 2P | BG7NUC-HC173 |
| 4258 | 2P | BG7NUC-HC174 |
| 4259 | 2P | BG7NUC-HC175 |
| 4260 | 2F | BG7NUC-HC176 |
| 4261 | 2P | BG7NUC-HC177 |
| 4262 | 2P | BG7NUC-HC178 |
| 4263 | 2P | BG7NUC-HC179 |
| 4264 | 2P | BG7NUC-HC180 |
| 4265 | 2P | BG7NUC-HC181 |
| 4266 | 2P | BG7NUC-HC182 |
| 4267 | 2P | BG7NUC-HC183 |
| 4268 | 2P | BG7NUC-HC184 |
| 4269 | 2P | BG7NUC-HC185 |
| 4270 | 2P | BG7NUC-HC186 |
| 4271 | 2F | BG7NUC-HC187 |
| 4272 | 2P | BG7NUC-HC188 |
| 4273 | 2P | BG7NUC-HC189 |
| 4274 | 2P | BG7NUC-HC190 |
| 4275 | 2P | BG7NUC-HC191 |
| 4276 | 2P | BG7NUC-HC192 |
| 4277 | 2P | BG7NUC-HC193 |
| 4278 | 2P | BG7NUC-HC194 |
| 4279 | 2P | BG7NUC-HC195 |
| 4280 | 2P | BG7NUC-HC196 |
| 4281 | 2P | BG7NUC-HC197 |
| 4282 | 2P | BG7NUC-HC198 |
| 4283 | 2P | BG7NUC-HC199 |
| 4284 | 2P | BG7NUC-HC200 |
| 4285 | 2P | BG7NUC-HC201 |
| 4286 | 2P | BG7NUC-HC202 |
| 4287 | 2P | BG7NUC-HC203 |
| 4288 | 2F | BG7NUC-HC204 |
| 4289 | 2P | BG7NUC-HC205 |
| 4290 | 2P | BG7NUC-HC206 |
| 4291 | 2P | BG7NUC-HC207 |
| 4292 | 2P | BG7NUC-HC208 |
| 4293 | 2F | BG7NUC-HC209 |
| 4294 | 2P | BG7NUC-HC210 |
| 4295 | 2P | BG7NUC-HC211 |
| 4296 | 2P | BG7NUC-HC212 |
| 4297 | 2P | BG7NUC-HC213 |
| 4298 | 2P | BG7NUC-HC214 |
| 4299 | 2P | BG7NUC-HC215 |
| 4300 | 2P | BG7NUC-HC216 |
| 4301 | 2P | BG7NUC-HC217 |
| 4302 | 2P | BG7NUC-HC218 |
| 4303 | 2P | BG7NUC-HC219 |
| 4304 | 2P | BG7NUC-HC220 |
| 4305 | 2F | BG7NUC-HC221 |
| 4306 | 2P | BG7NUC-HC222 |
| 4307 | 2P | BG7NUC-HC223 |
| 4308 | 2P | BG7NUC-HC224 |
| 4309 | 2F | BG7NUC-HC225 |
| 4310 | 2P | BG7NUC-HC226 |
| 4311 | 2P | BG7NUC-HC227 |
| 4312 | 2P | BG7NUC-HC228 |
| 4313 | 2P | BG7NUC-HC229 |
| 4314 | 2P | BG7NUC-HC230 |
| 4315 | 2P | BG7NUC-HC231 |
| 4316 | 2P | BG7NUC-HC232 |
| 4317 | 2P | BG7NUC-HC233 |
| 4318 | 2F | BG7NUC-HC234 |
| 4319 | 2P | BG7NUC-HC235 |
| 4320 | 2P | BG7NUC-HC236 |
| 4321 | 2P | BG7NUC-HC237 |
| 4322 | 2P | BG7NUC-HC238 |
| 4323 | 2P | BG7NUC-HC239 |
| 4324 | 2F | BG7NUC-HC240 |
| 4325 | 2P | BG7NUC-HC241 |
| 4326 | 2P | BG7NUC-HC242 |
| 4327 | 2P | BG7NUC-HC243 |
| 4328 | 2P | BG7NUC-HC244 |
| 4329 | 2P | BG7NUC-HC245 |
| 4330 | 2P | BG7NUC-HC246 |
| 4331 | 2P | BG7NUC-HC247 |
| 4332 | 2P | BG7NUC-HC248 |
| 4333 | 2P | BG7NUC-HC249 |
| 4334 | 2P | BG7NUC-HC250 |
| 4335 | 2P | BG7NUC-HC251 |
| 4336 | 2P | BG7NUC-HC252 |
| 4337 | 2P | BG7NUC-HC253 |
| 4338 | RBD | BG7NUC-HC254 |
| 4339 | RBD | BG7NUC-HC255 |
| 4340 | RBD | BG7NUC-HC256 |
| 4341 | RBD | BG7NUC-HC257 |
| 4342 | RBD | BG7NUC-HC258 |
| 4343 | RBD | BG7NUC-HC259 |
| 4344 | RBD | BG7NUC-HC260 |
| 4345 | RBD | BG7NUC-HC261 |
| 4346 | RBD | BG7NUC-HC262 |
| 4347 | RBD | BG7NUC-HC263 |
| 4348 | RBD | BG7NUC-HC264 |
| 4349 | RBD | BG7NUC-HC265 |
| 4350 | RBD | BG7NUC-HC266 |
| 4351 | RBD | BG7NUC-HC267 |
| 4352 | RBD | BG7NUC-HC268 |
| 4353 | RBD | BG7NUC-HC269 |
| 4354 | RBD | BG7NUC-HC270 |
| 4355 | RBD | BG7NUC-HC271 |
| 4356 | RBD | BG7NUC-HC272 |
| 4357 | RBD | BG7NUC-HC273 |
| 4358 | RBD | BG7NUC-HC274 |
| 4359 | RBD | BG7NUC-HC275 |
| 4360 | RBD | BG7NUC-HC276 |
| 4361 | RBD | BG7NUC-HC277 |
| 4362 | RBD | BG7NUC-HC278 |
| 4363 | RBD | BG7NUC-HC279 |
| 4364 | RBD | BG7NUC-HC280 |
| 4365 | RBD | BG7NUC-HC281 |
| 4366 | RBD | BG7NUC-HC282 |
| 4367 | RBD | BG7NUC-HC283 |
| 4368 | RBD | BG7NUC-HC284 |
| 4369 | RBD | BG7NUC-HC285 |
| 4370 | RBD | BG7NUC-HC286 |
| 4371 | RBD | BG7NUC-HC287 |
| 4372 | RBD | BG7NUC-HC288 |
| 4373 | RBD | BG7NUC-HC289 |
| 4374 | RBD | BG7NUC-HC290 |
| 4375 | RBD | BG7NUC-HC291 |
| 4376 | RBD | BG7NUC-HC292 |
| 4377 | RBD | BG7NUC-HC293 |
| 4378 | RBD | BG7NUC-HC294 |
| 4379 | RBD | BG7NUC-HC295 |
| 4380 | RBD | BG7NUC-HC296 |
| 4381 | RBD | BG7NUC-HC297 |
| 4382 | RBD | BG7NUC-HC298 |
| 4383 | RBD | BG7NUC-HC299 |
| 4384 | RBD | BG7NUC-HC300 |
| 4385 | RBD | BG7NUC-HC301 |
| 4386 | RBD | BG7NUC-HC302 |
| 4387 | RBD | BG7NUC-HC303 |
| 4388 | RBD | BG7NUC-HC304 |
| 4389 | RBD | BG7NUC-HC305 |
| 4390 | RBD | BG7NUC-HC306 |
| 4391 | RBD | BG7NUC-HC307 |
| 4392 | RBD | BG7NUC-HC308 |
| 4393 | RBD | BG7NUC-HC309 |
| 4394 | RBD | BG7NUC-HC310 |
| 4395 | RBD | BG7NUC-HC311 |
| 4396 | RBD | BG7NUC-HC312 |
| 4397 | RBD | BG7NUC-HC313 |
| 4398 | RBD | BG7NUC-HC314 |
| 4399 | RBD | BG7NUC-HC315 |
| 4400 | RBD | BG7NUC-HC316 |
| 4401 | RBD | BG7NUC-HC317 |
| 4402 | RBD | BG7NUC-HC318 |
| 4403 | RBD | BG7NUC-HC319 |
| 4404 | RBD | BG7NUC-HC320 |
| 4405 | RBD | BG7NUC-HC321 |
| 4406 | RBD | BG7NUC-HC322 |
| 4407 | RBD | BG7NUC-HC323 |
| 4408 | RBD | BG7NUC-HC324 |
| 4409 | RBD | BG7NUC-HC325 |
| 4410 | RBD | BG7NUC-HC326 |
| 4411 | RBD | BG7NUC-HC327 |
| 4412 | RBD | BG7NUC-HC328 |
| 4413 | RBD | BG7NUC-HC329 |
| 4414 | RBD | BG7NUC-HC330 |
| 4415 | RBD | BG7NUC-HC331 |
| 4416 | RBD | BG7NUC-HC332 |
| 4417 | RBD | BG7NUC-HC333 |
| 4418 | RBD | BG7NUC-HC334 |
| 4419 | RBD | BG7NUC-HC335 |
| 4420 | RBD | BG7NUC-HC336 |
| 4421 | RBD | BG7NUC-HC337 |
| 4422 | RBD | BG7NUC-HC338 |
| 4423 | RBD | BG7NUC-HC339 |
| 4424 | RBD | BG7NUC-HC340 |
| 4425 | RBD | BG7NUC-HC341 |
| 4426 | RBD | BG7NUC-HC342 |
| 4427 | RBD | BG7NUC-HC343 |
| 4428 | RBD | BG7NUC-HC344 |
| 4429 | RBD | BG7NUC-HC345 |
| 4430 | RBD | BG7NUC-HC346 |
| 4431 | RBD | BG7NUC-HC347 |
| 4432 | RBD | BG7NUC-HC348 |
| 4433 | RBD | BG7NUC-HC349 |
| 4434 | RBD | BG7NUC-HC350 |
| 4435 | RBD | BG7NUC-HC351 |
| 4436 | RBD | BG7NUC-HC352 |
| 4437 | RBD | BG7NUC-HC353 |
| 4438 | RBD | BG7NUC-HC354 |
| 4439 | RBD | BG7NUC-HC355 |
| 4440 | RBD | BG7NUC-HC356 |
| 4441 | RBD | BG7NUC-HC357 |
| 4442 | RBD | BG7NUC-HC358 |
| 4443 | RBD | BG7NUC-HC359 |
| 4444 | RBD | BG7NUC-HC360 |
| 4445 | RBD | BG7NUC-HC361 |
| 4446 | RBD | BG7NUC-HC362 |
| 4447 | RBD | BG7NUC-HC363 |
| 4448 | RBD | BG7NUC-HC364 |
| 4449 | RBD | BG7NUC-HC365 |
| 4450 | RBD | BG7NUC-HC366 |
| 4451 | RBD | BG7NUC-HC367 |
| 4452 | RBD | BG7NUC-HC368 |
| 4453 | RBD | BG7NUC-HC369 |
| 4454 | RBD | BG7NUC-HC370 |
| 4455 | RBD | BG7NUC-HC371 |
| 4456 | RBD | BG7NUC-HC372 |
| 4457 | RBD | BG7NUC-HC373 |
| 4458 | RBD | BG7NUC-HC374 |
| 4459 | RBD | BG7NUC-HC375 |
| 4460 | RBD | BG7NUC-HC376 |
| 4461 | RBD | BG7NUC-HC377 |
| 4462 | RBD | BG7NUC-HC378 |
| 4463 | RBD | BG7NUC-HC379 |
| 4464 | RBD | BG7NUC-HC380 |
| 4465 | RBD | BG7NUC-HC381 |
| 4466 | RBD | BG7NUC-HC382 |
| 4467 | RBD | BG7NUC-HC383 |
| 4468 | RBD | BG7NUC-HC384 |
| 4469 | RBD | BG7NUC-HC385 |
| 4470 | RBD | BG7NUC-HC386 |
| 4471 | RBD | BG7NUC-HC387 |
| 4472 | RBD | BG7NUC-HC388 |
| 4473 | RBD | BG7NUC-HC389 |
| 4474 | RBD | BG7NUC-HC390 |
| 4475 | RBD | BG7NUC-HC391 |
| 4476 | RBD | BG7NUC-HC392 |
| 4477 | RBD | BG7NUC-HC393 |
| 4478 | RBD | BG7NUC-HC394 |
| 4479 | RBD | BG7NUC-HC395 |
| 4480 | RBD | BG7NUC-HC396 |
| 4481 | RBD | BG7NUC-HC397 |
| 4482 | 2P | BG7NUC-LC1 |
| 4483 | 2P | BG7NUC-LC2 |
| 4484 | 2P | BG7NUC-LC3 |
| 4485 | 2P | BG7NUC-LC4 |
| 4486 | 2P | BG7NUC-LC5 |
| 4487 | 2P | BG7NUC-LC6 |
| 4488 | 2P | BG7NUC-LC7 |
| 4489 | 2P | BG7NUC-LC8 |
| 4490 | 2P | BG7NUC-LC9 |
| 4491 | 2P | BG7NUC-LC10 |
| 4492 | 2P | BG7NUC-LC11 |
| 4493 | 2P | BG7NUC-LC12 |
| 4494 | 2P | BG7NUC-LC13 |
| 4495 | 2P | BG7NUC-LC14 |
| 4496 | 2P | BG7NUC-LC15 |
| 4497 | 2P | BG7NUC-LC16 |
| 4498 | 2P | BG7NUC-LC17 |
| 4499 | 2P | BG7NUC-LC18 |
| 4500 | 2P | BG7NUC-LC19 |
| 4501 | 2P | BG7NUC-LC20 |
| 4502 | 2P | BG7NUC-LC21 |
| 4503 | 2P | BG7NUC-LC22 |
| 4504 | 2P | BG7NUC-LC23 |
| 4505 | 2P | BG7NUC-LC24 |
| 4506 | 2P | BG7NUC-LC25 |
| 4507 | 2P | BG7NUC-LC26 |
| 4508 | 2P | BG7NUC-LC27 |
| 4509 | 2P | BG7NUC-LC28 |
| 4510 | 2P | BG7NUC-LC29 |
| 4511 | 2P | BG7NUC-LC30 |
| 4512 | 2P | BG7NUC-LC31 |
| 4513 | 2P | BG7NUC-LC32 |
| 4514 | RBD | BG7NUC-LC33 |
| 4515 | RBD | BG7NUC-LC34 |
| 4516 | RBD | BG7NUC-LC35 |
| 4517 | RBD | BG7NUC-LC36 |
| 4518 | 2P | BG7NUC-LC37 |
| 4519 | 2P | BG7NUC-LC38 |
| 4520 | 2P | BG7NUC-LC39 |
| 4521 | 2P | BG7NUC-LC40 |
| 4522 | 2P | BG7NUC-LC41 |
| 4523 | 2P | BG7NUC-LC42 |
| 4524 | 2P | BG7NUC-LC43 |
| 4525 | 2P | BG7NUC-LC44 |
| 4526 | 2P | BG7NUC-LC45 |
| 4527 | 2P | BG7NUC-LC46 |
| 4528 | 2P | BG7NUC-LC47 |
| 4529 | 2P | BG7NUC-LC48 |
| 4530 | 2P | BG7NUC-LC49 |
| 4531 | 2P | BG7NUC-LC50 |
| 4532 | 2P | BG7NUC-LC51 |
| 4533 | 2P | BG7NUC-LC52 |
| 4534 | 2P | BG7NUC-LC53 |
| 4535 | 2P | BG7NUC-LC54 |
| 4536 | 2P | BG7NUC-LC55 |
| 4537 | 2P | BG7NUC-LC56 |
| 4538 | 2P | BG7NUC-LC57 |
| 4539 | 2P | BG7NUC-LC58 |
| 4540 | 2P | BG7NUC-LC59 |
| 4541 | 2P | BG7NUC-LC60 |
| 4542 | RBD | BG7NUC-LC61 |
| 4543 | 2P | BG7NUC-LC62 |
| 4544 | 2P | BG7NUC-LC63 |
| 4545 | RBD | BG7NUC-LC64 |
| 4546 | RBD | BG7NUC-LC65 |
| 4547 | RBD | BG7NUC-LC66 |
| 4548 | RBD | BG7NUC-LC67 |
| 4549 | RBD | BG7NUC-LC68 |
| 4550 | 2P | BG7NUC-LC69 |
| 4551 | 2P | BG7NUC-LC70 |
| 4552 | 2P | BG7NUC-LC71 |
| 4553 | 2P | BG7NUC-LC72 |
| 4554 | 2P | BG7NUC-LC73 |
| 4555 | 2P | BG7NUC-LC74 |
| 4556 | 2P | BG7NUC-LC75 |
| 4557 | 2P | BG7NUC-LC76 |
| 4558 | 2P | BG7NUC-LC77 |
| 4559 | 2P | BG7NUC-LC78 |
| 4560 | 2P | BG7NUC-LC79 |
| 4561 | 2P | BG7NUC-LC80 |
| 4562 | 2F | BG7NUC-LC81 |
| 4563 | 2P | BG7NUC-LC82 |
| 4564 | 2P | BG7NUC-LC83 |
| 4565 | 2P | BG7NUC-LC84 |
| 4566 | 2P | BG7NUC-LC85 |
| 4567 | 2P | BG7NUC-LC86 |
| 4568 | 2P | BG7NUC-LC87 |
| 4569 | 2P | BG7NUC-LC88 |
| 4570 | 2P | BG7NUC-LC89 |
| 4571 | 2P | BG7NUC-LC90 |
| 4572 | 2P | BG7NUC-LC91 |
| 4573 | 2P | BG7NUC-LC92 |
| 4574 | 2P | BG7NUC-LC93 |
| 4575 | 2P | BG7NUC-LC94 |
| 4576 | 2P | BG7NUC-LC95 |
| 4577 | 2P | BG7NUC-LC96 |
| 4578 | 2P | BG7NUC-LC97 |
| 4579 | 2P | BG7NUC-LC98 |
| 4580 | 2P | BG7NUC-LC99 |
| 4581 | 2P | BG7NUC-LC100 |
| 4582 | 2P | BG7NUC-LC101 |
| 4583 | 2P | BG7NUC-LC102 |
| 4584 | 2P | BG7NUC-LC103 |
| 4585 | 2P | BG7NUC-LC104 |
| 4586 | 2P | BG7NUC-LC105 |
| 4587 | 2P | BG7NUC-LC106 |
| 4588 | 2P | BG7NUC-LC107 |
| 4589 | 2P | BG7NUC-LC108 |
| 4590 | 2P | BG7NUC-LC109 |
| 4591 | 2P | BG7NUC-LC110 |
| 4592 | 2P | BG7NUC-LC111 |
| 4593 | 2P | BG7NUC-LC112 |
| 4594 | 2P | BG7NUC-LC113 |
| 4595 | 2P | BG7NUC-LC114 |
| 4596 | 2P | BG7NUC-LC115 |
| 4597 | 2P | BG7NUC-LC116 |
| 4598 | 2P | BG7NUC-LC117 |
| 4599 | 2P | BG7NUC-LC118 |
| 4600 | 2P | BG7NUC-LC119 |
| 4601 | 2P | BG7NUC-LC120 |
| 4602 | 2P | BG7NUC-LC121 |
| 4603 | 2P | BG7NUC-LC122 |
| 4604 | 2P | BG7NUC-LC123 |
| 4605 | 2P | BG7NUC-LC124 |
| 4606 | 2P | BG7NUC-LC125 |
| 4607 | 2P | BG7NUC-LC126 |
| 4608 | 2F | BG7NUC-LC127 |
| 4609 | 2P | BG7NUC-LC128 |
| 4610 | 2P | BG7NUC-LC129 |
| 4611 | 2P | BG7NUC-LC130 |
| 4612 | 2P | BG7NUC-LC131 |
| 4613 | 2P | BG7NUC-LC132 |
| 4614 | 2P | BG7NUC-LC133 |
| 4615 | 2P | BG7NUC-LC134 |
| 4616 | 2P | BG7NUC-LC135 |
| 4617 | 2P | BG7NUC-LC136 |
| 4618 | 2P | BG7NUC-LC137 |
| 4619 | 2P | BG7NUC-LC138 |
| 4620 | 2P | BG7NUC-LC139 |
| 4621 | 2P | BG7NUC-LC140 |
| 4622 | 2P | BG7NUC-LC141 |
| 4623 | 2P | BG7NUC-LC142 |
| 4624 | 2P | BG7NUC-LC143 |
| 4625 | 2P | BG7NUC-LC144 |
| 4626 | 2P | BG7NUC-LC145 |
| 4627 | 2P | BG7NUC-LC146 |
| 4628 | 2P | BG7NUC-LC147 |
| 4629 | 2P | BG7NUC-LC148 |
| 4630 | 2P | BG7NUC-LC149 |
| 4631 | 2P | BG7NUC-LC150 |
| 4632 | 2P | BG7NUC-LC151 |
| 4633 | 2P | BG7NUC-LC152 |
| 4634 | 2P | BG7NUC-LC153 |
| 4635 | 2P | BG7NUC-LC154 |
| 4636 | 2P | BG7NUC-LC155 |
| 4637 | 2P | BG7NUC-LC156 |
| 4638 | 2P | BG7NUC-LC157 |
| 4639 | 2P | BG7NUC-LC158 |
| 4640 | 2P | BG7NUC-LC159 |
| 4641 | 2P | BG7NUC-LC160 |
| 4642 | 2P | BG7NUC-LC161 |
| 4643 | 2P | BG7NUC-LC162 |
| 4644 | 2P | BG7NUC-LC163 |
| 4645 | 2P | BG7NUC-LC164 |
| 4646 | 2P | BG7NUC-LC165 |
| 4647 | 2P | BG7NUC-LC166 |
| 4648 | 2P | BG7NUC-LC167 |
| 4649 | 2P | BG7NUC-LC168 |
| 4650 | 2P | BG7NUC-LC169 |
| 4651 | 2P | BG7NUC-LC170 |
| 4652 | 2P | BG7NUC-LC171 |
| 4653 | 2P | BG7NUC-LC172 |
| 4654 | 2P | BG7NUC-LC173 |
| 4655 | 2P | BG7NUC-LC174 |
| 4656 | 2P | BG7NUC-LC175 |
| 4657 | 2P | BG7NUC-LC176 |
| 4658 | 2P | BG7NUC-LC177 |
| 4659 | 2P | BG7NUC-LC178 |
| 4660 | 2P | BG7NUC-LC179 |
| 4661 | 2P | BG7NUC-LC180 |
| 4662 | 2P | BG7NUC-LC181 |
| 4663 | 2P | BG7NUC-LC182 |
| 4664 | 2P | BG7NUC-LC183 |
| 4665 | 2P | BG7NUC-LC184 |
| 4666 | 2P | BG7NUC-LC185 |
| 4667 | 2P | BG7NUC-LC186 |
| 4668 | 2P | BG7NUC-LC187 |
| 4669 | 2P | BG7NUC-LC188 |
| 4670 | 2P | BG7NUC-LC189 |
| 4671 | 2P | BG7NUC-LC190 |
| 4672 | 2P | BG7NUC-LC191 |
| 4673 | 2P | BG7NUC-LC192 |
| 4674 | 2P | BG7NUC-LC193 |
| 4675 | 2P | BG7NUC-LC194 |
| 4676 | 2P | BG7NUC-LC195 |
| 4677 | 2P | BG7NUC-LC196 |
| 4678 | 2P | BG7NUC-LC197 |
| 4679 | 2P | BG7NUC-LC198 |
| 4680 | 2P | BG7NUC-LC199 |
| 4681 | 2P | BG7NUC-LC200 |
| 4682 | 2P | BG7NUC-LC201 |
| 4683 | 2P | BG7NUC-LC202 |
| 4684 | 2P | BG7NUC-LC203 |
| 4685 | 2P | BG7NUC-LC204 |
| 4686 | 2P | BG7NUC-LC205 |
| 4687 | 2P | BG7NUC-LC206 |
| 4688 | 2P | BG7NUC-LC207 |
| 4689 | 2P | BG7NUC-LC208 |
| 4690 | 2P | BG7NUC-LC209 |
| 4691 | 2P | BG7NUC-LC210 |
| 4692 | 2P | BG7NUC-LC211 |
| 4693 | 2P | BG7NUC-LC212 |
| 4694 | 2P | BG7NUC-LC213 |
| 4695 | 2P | BG7NUC-LC214 |
| 4696 | 2P | BG7NUC-LC215 |
| 4697 | 2P | BG7NUC-LC216 |
| 4698 | 2P | BG7NUC-LC217 |
| 4699 | 2P | BG7NUC-LC218 |
| 4700 | 2P | BG7NUC-LC219 |
| 4701 | 2P | BG7NUC-LC220 |
| 4702 | 2P | BG7NUC-LC221 |
| 4703 | 2P | BG7NUC-LC222 |
| 4704 | 2P | BG7NUC-LC223 |
| 4705 | 2P | BG7NUC-LC224 |
| 4706 | 2P | BG7NUC-LC225 |
| 4707 | 2P | BG7NUC-LC226 |
| 4708 | 2P | BG7NUC-LC227 |
| 4709 | 2P | BG7NUC-LC228 |
| 4710 | 2P | BG7NUC-LC229 |
| 4711 | 2P | BG7NUC-LC230 |
| 4712 | 2P | BG7NUC-LC231 |
| 4713 | 2P | BG7NUC-LC232 |
| 4714 | 2P | BG7NUC-LC233 |
| 4715 | 2P | BG7NUC-LC234 |
| 4716 | 2P | BG7NUC-LC235 |
| 4717 | 2P | BG7NUC-LC236 |
| 4718 | 2P | BG7NUC-LC237 |
| 4719 | 2P | BG7NUC-LC238 |
| 4720 | 2P | BG7NUC-LC239 |
| 4721 | 2P | BG7NUC-LC240 |
| 4722 | 2P | BG7NUC-LC241 |
| 4723 | 2P | BG7NUC-LC242 |
| 4724 | 2P | BG7NUC-LC243 |
| 4725 | 2P | BG7NUC-LC244 |
| 4726 | 2P | BG7NUC-LC245 |
| 4727 | 2P | BG7NUC-LC246 |
| 4728 | 2P | BG7NUC-LC247 |
| 4729 | 2P | BG7NUC-LC248 |
| 4730 | 2P | BG7NUC-LC249 |
| 4731 | 2P | BG7NUC-LC250 |
| 4732 | 2P | BG7NUC-LC251 |
| 4733 | 2P | BG7NUC-LC252 |
| 4734 | 2P | BG7NUC-LC253 |
| 4735 | RBD | BG7NUC-LC254 |
| 4736 | RBD | BG7NUC-LC255 |
| 4737 | RBD | BG7NUC-LC256 |
| 4738 | RBD | BG7NUC-LC257 |
| 4739 | RBD | BG7NUC-LC258 |
| 4740 | RBD | BG7NUC-LC259 |
| 4741 | RBD | BG7NUC-LC260 |
| 4742 | RBD | BG7NUC-LC261 |
| 4743 | RBD | BG7NUC-LC262 |
| 4744 | RBD | BG7NUC-LC263 |
| 4745 | RBD | BG7NUC-LC264 |
| 4746 | RBD | BG7NUC-LC265 |
| 4747 | RBD | BG7NUC-LC266 |
| 4748 | RBD | BG7NUC-LC267 |
| 4749 | RBD | BG7NUC-LC268 |
| 4750 | RBD | BG7NUC-LC269 |
| 4751 | RBD | BG7NUC-LC270 |
| 4752 | RBD | BG7NUC-LC271 |
| 4753 | RBD | BG7NUC-LC272 |
| 4754 | RBD | BG7NUC-LC273 |
| 4755 | RBD | BG7NUC-LC274 |
| 4756 | RBD | BG7NUC-LC275 |
| 4757 | RBD | BG7NUC-LC276 |
| 4758 | RBD | BG7NUC-LC277 |
| 4759 | RBD | BG7NUC-LC278 |
| 4760 | RBD | BG7NUC-LC279 |
| 4761 | RBD | BG7NUC-LC280 |
| 4762 | RBD | BG7NUC-LC281 |
| 4763 | RBD | BG7NUC-LC282 |
| 4764 | RBD | BG7NUC-LC283 |
| 4765 | RBD | BG7NUC-LC284 |
| 4766 | RBD | BG7NUC-LC285 |
| 4767 | RBD | BG7NUC-LC286 |
| 4768 | RBD | BG7NUC-LC287 |
| 4769 | RBD | BG7NUC-LC288 |
| 4770 | RBD | BG7NUC-LC289 |
| 4771 | RBD | BG7NUC-LC290 |
| 4772 | RBD | BG7NUC-LC291 |
| 4773 | RBD | BG7NUC-LC292 |
| 4774 | RBD | BG7NUC-LC293 |
| 4775 | RBD | BG7NUC-LC294 |
| 4776 | RBD | BG7NUC-LC295 |
| 4777 | RBD | BG7NUC-LC296 |
| 4778 | RBD | BG7NUC-LC297 |
| 4779 | RBD | BG7NUC-LC298 |
| 4780 | RBD | BG7NUC-LC299 |
| 4781 | RBD | BG7NUC-LC300 |
| 4782 | RBD | BG7NUC-LC301 |
| 4783 | RBD | BG7NUC-LC302 |
| 4784 | RBD | BG7NUC-LC303 |
| 4785 | RBD | BG7NUC-LC304 |
| 4786 | RBD | BG7NUC-LC305 |
| 4787 | RBD | BG7NUC-LC306 |
| 4788 | RBD | BG7NUC-LC307 |
| 4789 | RBD | BG7NUC-LC308 |
| 4790 | RBD | BG7NUC-LC309 |
| 4791 | RBD | BG7NUC-LC310 |
| 4792 | RBD | BG7NUC-LC311 |
| 4793 | RBD | BG7NUC-LC312 |
| 4794 | RBD | BG7NUC-LC313 |
| 4795 | RBD | BG7NUC-LC314 |
| 4796 | RBD | BG7NUC-LC315 |
| 4797 | RBD | BG7NUC-LC316 |
| 4798 | RBD | BG7NUC-LC317 |
| 4799 | RBD | BG7NUC-LC318 |
| 4800 | RBD | BG7NUC-LC319 |
| 4801 | RBD | BG7NUC-LC320 |
| 4802 | RBD | BG7NUC-LC321 |
| 4803 | RBD | BG7NUC-LC322 |
| 4804 | RBD | BG7NUC-LC323 |
| 4805 | RBD | BG7NUC-LC324 |
| 4806 | RBD | BG7NUC-LC325 |
| 4807 | RBD | BG7NUC-LC326 |
| 4808 | RBD | BG7NUC-LC327 |
| 4809 | RBD | BG7NUC-LC328 |
| 4810 | RBD | BG7NUC-LC329 |
| 4811 | RBD | BG7NUC-LC330 |
| 4812 | RBD | BG7NUC-LC331 |
| 4813 | RBD | BG7NUC-LC332 |
| 4814 | RBD | BG7NUC-LC333 |
| 4815 | RBD | BG7NUC-LC334 |
| 4816 | RBD | BG7NUC-LC335 |
| 4817 | RBD | BG7NUC-LC336 |
| 4818 | RBD | BG7NUC-LC337 |
| 4819 | RBD | BG7NUC-LC338 |
| 4820 | RBD | BG7NUC-LC339 |
| 4821 | RBD | BG7NUC-LC340 |
| 4822 | RBD | BG7NUC-LC341 |
| 4823 | RBD | BG7NUC-LC342 |
| 4824 | RBD | BG7NUC-LC343 |
| 4825 | RBD | BG7NUC-LC344 |
| 4826 | RBD | BG7NUC-LC345 |
| 4827 | RBD | BG7NUC-LC346 |
| 4828 | RBD | BG7NUC-LC347 |
| 4829 | RBD | BG7NUC-LC348 |
| 4830 | RBD | BG7NUC-LC349 |
| 4831 | RBD | BG7NUC-LC350 |
| 4832 | RBD | BG7NUC-LC351 |
| 4833 | RBD | BG7NUC-LC352 |
| 4834 | RBD | BG7NUC-LC353 |
| 4835 | RBD | BG7NUC-LC354 |
| 4836 | RBD | BG7NUC-LC355 |
| 4837 | RBD | BG7NUC-LC356 |
| 4838 | RBD | BG7NUC-LC357 |
| 4839 | RBD | BG7NUC-LC358 |
| 4840 | RBD | BG7NUC-LC359 |
| 4841 | RBD | BG7NUC-LC360 |
| 4842 | RBD | BG7NUC-LC361 |
| 4843 | RBD | BG7NUC-LC362 |
| 4844 | RBD | BG7NUC-LC363 |
| 4845 | RBD | BG7NUC-LC364 |
| 4846 | RBD | BG7NUC-LC365 |
| 4847 | RBD | BG7NUC-LC366 |
| 4848 | RBD | BG7NUC-LC367 |
| 4849 | RBD | BG7NUC-LC368 |
| 4850 | RBD | BG7NUC-LC369 |
| 4851 | RBD | BG7NUC-LC370 |
| 4852 | RBD | BG7NUC-LC371 |
| 4853 | RBD | BG7NUC-LC372 |
| 4854 | RBD | BG7NUC-LC373 |
| 4855 | RBD | BG7NUC-LC374 |
| 4856 | RBD | BG7NUC-LC375 |
| 4857 | RBD | BG7NUC-LC376 |
| 4858 | RBD | BG7NUC-LC377 |
| 4859 | RBD | BG7NUC-LC378 |
| 4860 | RBD | BG7NUC-LC379 |
| 4861 | RBD | BG7NUC-LC380 |
| 4862 | RBD | BG7NUC-LC381 |
| 4863 | RBD | BG7NUC-LC382 |
| 4864 | RBD | BG7NUC-LC383 |
| 4865 | RBD | BG7NUC-LC384 |
| 4866 | RBD | BG7NUC-LC385 |
| 4867 | RBD | BG7NUC-LC386 |
| 4868 | RBD | BG7NUC-LC387 |
| 4869 | RBD | BG7NUC-LC388 |
| 4870 | RBD | BG7NUC-LC389 |
| 4871 | RBD | BG7NUC-LC390 |
| 4872 | RBD | BG7NUC-LC391 |
| 4873 | RBD | BG7NUC-LC392 |
| 4874 | RBD | BG7NUC-LC393 |
| 4875 | RBD | BG7NUC-LC394 |
| 4876 | RBD | BG7NUC-LC395 |
| 4877 | RBD | BG7NUC-LC396 |
| 4878 | RBD | BG7NUC-LC397 |
| 4879 | 2P | BG9NUC-HC1 |
| 4880 | 2P | BG9NUC-HC2 |
| 4881 | 2P | BG9NUC-HC3 |
| 4882 | 2P | BG9NUC-HC4 |
| 4883 | 2P | BG9NUC-HC5 |
| 4884 | 2P | BG9NUC-HC6 |
| 4885 | 2P | BG9NUC-HC7 |
| 4886 | RBD | BG9NUC-HC8 |
| 4887 | RBD | BG9NUC-HC9 |
| 4888 | RBD | BG9NUC-HC10 |
| 4889 | 2P | BG9NUC-HC11 |
| 4890 | RBD | BG9NUC-HC12 |
| 4891 | RBD | BG9NUC-HC13 |
| 4892 | RBD | BG9NUC-HC14 |
| 4893 | RBD | BG9NUC-HC15 |
| 4894 | 2P | BG9NUC-HC16 |
| 4895 | 2P | BG9NUC-HC17 |
| 4896 | 2P | BG9NUC-HC18 |
| 4897 | 2P | BG9NUC-HC19 |
| 4898 | 2P | BG9NUC-HC20 |
| 4899 | 2P | BG9NUC-HC21 |
| 4900 | 2P | BG9NUC-HC22 |
| 4901 | RBD | BG9NUC-HC23 |
| 4902 | 2P | BG9NUC-HC24 |
| 4903 | RBD | BG9NUC-HC25 |
| 4904 | RBD | BG9NUC-HC26 |
| 4905 | RBD | BG9NUC-HC27 |
| 4906 | RBD | BG9NUC-HC28 |
| 4907 | 2P | BG9NUC-HC29 |
| 4908 | 2P | BG9NUC-HC30 |
| 4909 | RBD | BG9NUC-HC31 |
| 4910 | 2P | BG9NUC-HC32 |
| 4911 | RBD | BG9NUC-HC33 |
| 4912 | RBD | BG9NUC-HC34 |
| 4913 | RBD | BG9NUC-HC35 |
| 4914 | RBD | BG9NUC-HC36 |
| 4915 | 2P | BG9NUC-HC37 |
| 4916 | 2P | BG9NUC-HC38 |
| 4917 | 2P | BG9NUC-HC39 |
| 4918 | RBD | BG9NUC-HC40 |
| 4919 | 2P | BG9NUC-HC41 |
| 4920 | 2P | BG9NUC-HC42 |
| 4921 | RBD | BG9NUC-HC43 |
| 4922 | 2P | BG9NUC-HC44 |
| 4923 | 2P | BG9NUC-HC45 |
| 4924 | RBD | BG9NUC-HC46 |
| 4925 | 2P | BG9NUC-HC47 |
| 4926 | 2P | BG9NUC-HC48 |
| 4927 | RBD | BG9NUC-HC49 |
| 4928 | 2P | BG9NUC-HC50 |
| 4929 | 2P | BG9NUC-HC51 |
| 4930 | RBD | BG9NUC-HC52 |
| 4931 | 2P | BG9NUC-HC53 |
| 4932 | RBD | BG9NUC-HC54 |
| 4933 | RBD | BG9NUC-HC55 |
| 4934 | 2P | BG9NUC-HC56 |
| 4935 | 2P | BG9NUC-HC57 |
| 4936 | RBD | BG9NUC-HC58 |
| 4937 | RBD | BG9NUC-HC59 |
| 4938 | RBD | BG9NUC-HC60 |
| 4939 | 2P | BG9NUC-HC61 |
| 4940 | 2P | BG9NUC-HC62 |
| 4941 | RBD | BG9NUC-HC63 |
| 4942 | 2P | BG9NUC-HC64 |
| 4943 | 2P | BG9NUC-HC65 |
| 4944 | 2P | BG9NUC-HC66 |
| 4945 | 2P | BG9NUC-HC67 |
| 4946 | 2P | BG9NUC-HC68 |
| 4947 | RBD | BG9NUC-HC69 |
| 4948 | 2P | BG9NUC-HC70 |
| 4949 | 2P | BG9NUC-HC71 |
| 4950 | RBD | BG9NUC-HC72 |
| 4951 | 2P | BG9NUC-HC73 |
| 4952 | 2P | BG9NUC-HC74 |
| 4953 | 2P | BG9NUC-HC75 |
| 4954 | RBD | BG9NUC-HC76 |
| 4955 | RBD | BG9NUC-HC77 |
| 4956 | RBD | BG9NUC-HC78 |
| 4957 | 2P | BG9NUC-HC79 |
| 4958 | 2P | BG9NUC-HC80 |
| 4959 | 2P | BG9NUC-HC81 |
| 4960 | 2P | BG9NUC-HC82 |
| 4961 | RBD | BG9NUC-HC83 |
| 4962 | RBD | BG9NUC-HC84 |
| 4963 | RBD | BG9NUC-HC85 |
| 4964 | RBD | BG9NUC-HC86 |
| 4965 | 2P | BG9NUC-HC87 |
| 4966 | 2P | BG9NUC-HC88 |
| 4967 | 2P | BG9NUC-HC89 |
| 4968 | 2P | BG9NUC-HC90 |
| 4969 | 2P | BG9NUC-HC91 |
| 4970 | RBD | BG9NUC-HC92 |
| 4971 | 2P | BG9NUC-HC93 |
| 4972 | 2P | BG9NUC-HC94 |
| 4973 | RBD | BG9NUC-HC95 |
| 4974 | 2P | BG9NUC-HC96 |
| 4975 | 2P | BG9NUC-HC97 |
| 4976 | 2P | BG9NUC-HC98 |
| 4977 | 2P | BG9NUC-HC99 |
| 4978 | RBD | BG9NUC-HC100 |
| 4979 | RBD | BG9NUC-HC101 |
| 4980 | RBD | BG9NUC-HC102 |
| 4981 | 2P | BG9NUC-HC103 |
| 4982 | RBD | BG9NUC-HC104 |
| 4983 | 2P | BG9NUC-HC105 |
| 4984 | 2P | BG9NUC-HC106 |
| 4985 | RBD | BG9NUC-HC107 |
| 4986 | RBD | BG9NUC-HC108 |
| 4987 | RBD | BG9NUC-HC109 |
| 4988 | 2P | BG9NUC-HC110 |
| 4989 | 2P | BG9NUC-HC111 |
| 4990 | 2P | BG9NUC-HC112 |
| 4991 | RBD | BG9NUC-HC113 |
| 4992 | 2P | BG9NUC-HC114 |
| 4993 | 2P | BG9NUC-HC115 |
| 4994 | RBD | BG9NUC-HC116 |
| 4995 | RBD | BG9NUC-HC117 |
| 4996 | RBD | BG9NUC-HC118 |
| 4997 | 2P | BG9NUC-HC119 |
| 4998 | 2P | BG9NUC-HC120 |
| 4999 | RBD | BG9NUC-HC121 |
| 5000 | RBD | BG9NUC-HC122 |
| 5001 | 2P | BG9NUC-HC123 |
| 5002 | 2P | BG9NUC-HC124 |
| 5003 | 2P | BG9NUC-HC125 |
| 5004 | 2P | BG9NUC-HC126 |
| 5005 | RBD | BG9NUC-HC127 |
| 5006 | 2P | BG9NUC-HC128 |
| 5007 | RBD | BG9NUC-HC129 |
| 5008 | RBD | BG9NUC-HC130 |
| 5009 | RBD | BG9NUC-HC131 |
| 5010 | RBD | BG9NUC-HC132 |
| 5011 | 2P | BG9NUC-HC133 |
| 5012 | RBD | BG9NUC-HC134 |
| 5013 | 2P | BG9NUC-HC135 |
| 5014 | RBD | BG9NUC-HC136 |
| 5015 | 2P | BG9NUC-HC137 |
| 5016 | RBD | BG9NUC-HC138 |
| 5017 | 2P | BG9NUC-HC139 |
| 5018 | 2P | BG9NUC-HC140 |
| 5019 | 2P | BG9NUC-HC141 |
| 5020 | 2P | BG9NUC-HC142 |
| 5021 | 2P | BG9NUC-HC143 |
| 5022 | 2P | BG9NUC-HC144 |
| 5023 | 2P | BG9NUC-HC145 |
| 5024 | 2P | BG9NUC-HC146 |
| 5025 | RBD | BG9NUC-HC147 |
| 5026 | RBD | BG9NUC-HC148 |
| 5027 | RBD | BG9NUC-HC149 |
| 5028 | RBD | BG9NUC-HC150 |
| 5029 | 2P | BG9NUC-HC151 |
| 5030 | 2P | BG9NUC-HC152 |
| 5031 | RBD | BG9NUC-HC153 |
| 5032 | 2P | BG9NUC-HC154 |
| 5033 | 2P | BG9NUC-HC155 |
| 5034 | 2P | BG9NUC-HC156 |
| 5035 | 2P | BG9NUC-HC157 |
| 5036 | RBD | BG9NUC-HC158 |
| 5037 | 2P | BG9NUC-HC159 |
| 5038 | 2P | BG9NUC-HC160 |
| 5039 | 2P | BG9NUC-HC161 |
| 5040 | RBD | BG9NUC-HC162 |
| 5041 | 2P | BG9NUC-HC163 |
| 5042 | 2P | BG9NUC-HC164 |
| 5043 | RBD | BG9NUC-HC165 |
| 5044 | 2P | BG9NUC-HC166 |
| 5045 | RBD | BG9NUC-HC167 |
| 5046 | RBD | BG9NUC-HC168 |
| 5047 | RBD | BG9NUC-HC169 |
| 5048 | RBD | BG9NUC-HC170 |
| 5049 | RBD | BG9NUC-HC171 |
| 5050 | 2P | BG9NUC-HC172 |
| 5051 | RBD | BG9NUC-HC173 |
| 5052 | 2P | BG9NUC-HC174 |
| 5053 | RBD | BG9NUC-HC175 |
| 5054 | RBD | BG9NUC-HC176 |
| 5055 | 2P | BG9NUC-HC177 |
| 5056 | RBD | BG9NUC-HC178 |
| 5057 | 2P | BG9NUC-HC179 |
| 5058 | 2P | BG9NUC-HC180 |
| 5059 | 2P | BG9NUC-HC181 |
| 5060 | RBD | BG9NUC-HC182 |
| 5061 | 2P | BG9NUC-HC183 |
| 5062 | RBD | BG9NUC-HC184 |
| 5063 | 2P | BG9NUC-HC185 |
| 5064 | RBD | BG9NUC-HC186 |
| 5065 | 2P | BG9NUC-HC187 |
| 5066 | 2P | BG9NUC-HC188 |
| 5067 | 2P | BG9NUC-HC189 |
| 5068 | 2P | BG9NUC-HC190 |
| 5069 | 2P | BG9NUC-HC191 |
| 5070 | RBD | BG9NUC-HC192 |
| 5071 | 2P | BG9NUC-HC193 |
| 5072 | 2P | BG9NUC-HC194 |
| 5073 | RBD | BG9NUC-HC195 |
| 5074 | RBD | BG9NUC-HC196 |
| 5075 | 2P | BG9NUC-HC197 |
| 5076 | RBD | BG9NUC-HC198 |
| 5077 | 2P | BG9NUC-HC199 |
| 5078 | 2P | BG9NUC-HC200 |
| 5079 | RBD | BG9NUC-HC201 |
| 5080 | 2P | BG9NUC-HC202 |
| 5081 | 2P | BG9NUC-HC203 |
| 5082 | 2P | BG9NUC-HC204 |
| 5083 | 2P | BG9NUC-HC205 |
| 5084 | RBD | BG9NUC-HC206 |
| 5085 | RBD | BG9NUC-HC207 |
| 5086 | 2P | BG9NUC-HC208 |
| 5087 | RBD | BG9NUC-HC209 |
| 5088 | RBD | BG9NUC-HC210 |
| 5089 | 2P | BG9NUC-HC211 |
| 5090 | RBD | BG9NUC-HC212 |
| 5091 | RBD | BG9NUC-HC213 |
| 5092 | 2P | BG9NUC-HC214 |
| 5093 | 2P | BG9NUC-HC215 |
| 5094 | RBD | BG9NUC-HC216 |
| 5095 | 2P | BG9NUC-HC217 |
| 5096 | 2P | BG9NUC-HC218 |
| 5097 | RBD | BG9NUC-HC219 |
| 5098 | RBD | BG9NUC-HC220 |
| 5099 | 2P | BG9NUC-HC221 |
| 5100 | RBD | BG9NUC-HC222 |
| 5101 | 2P | BG9NUC-HC223 |
| 5102 | 2P | BG9NUC-HC224 |
| 5103 | RBD | BG9NUC-HC225 |
| 5104 | 2P | BG9NUC-HC226 |
| 5105 | RBD | BG9NUC-HC227 |
| 5106 | RBD | BG9NUC-HC228 |
| 5107 | 2P | BG9NUC-HC229 |
| 5108 | 2P | BG9NUC-HC230 |
| 5109 | 2P | BG9NUC-HC231 |
| 5110 | RBD | BG9NUC-HC232 |
| 5111 | RBD | BG9NUC-HC233 |
| 5112 | RBD | BG9NUC-HC234 |
| 5113 | 2P | BG9NUC-LC1 |
| 5114 | 2P | BG9NUC-LC2 |
| 5115 | 2P | BG9NUC-LC3 |
| 5116 | 2P | BG9NUC-LC4 |
| 5117 | 2P | BG9NUC-LC5 |
| 5118 | 2P | BG9NUC-LC6 |
| 5119 | 2P | BG9NUC-LC7 |
| 5120 | RBD | BG9NUC-LC8 |
| 5121 | RBD | BG9NUC-LC9 |
| 5122 | RBD | BG9NUC-LC10 |
| 5123 | 2F | BG9NUC-LC11 |
| 5124 | RBD | BG9NUC-LC12 |
| 5125 | RBD | BG9NUC-LC13 |
| 5126 | RBD | BG9NUC-LC14 |
| 5127 | RBD | BG9NUC-LC15 |
| 5128 | 2P | BG9NUC-LC16 |
| 5129 | 2P | BG9NUC-LC17 |
| 5130 | 2P | BG9NUC-LC18 |
| 5131 | 2P | BG9NUC-LC19 |
| 5132 | 2P | BG9NUC-LC20 |
| 5133 | 2P | BG9NUC-LC21 |
| 5134 | 2P | BG9NUC-LC22 |
| 5135 | RBD | BG9NUC-LC23 |
| 5136 | 2F | BG9NUC-LC24 |
| 5137 | RBD | BG9NUC-LC25 |
| 5138 | RBD | BG9NUC-LC26 |
| 5139 | RBD | BG9NUC-LC27 |
| 5140 | RBD | BG9NUC-LC28 |
| 5141 | 2F | BG9NUC-LC29 |
| 5142 | 2P | BG9NUC-LC30 |
| 5143 | RBD | BG9NUC-LC31 |
| 5144 | 2P | BG9NUC-LC32 |
| 5145 | RBD | BG9NUC-LC33 |
| 5146 | RBD | BG9NUC-LC34 |
| 5147 | RBD | BG9NUC-LC35 |
| 5148 | RBD | BG9NUC-LC36 |
| 5149 | 2P | BG9NUC-LC37 |
| 5150 | 2P | BG9NUC-LC38 |
| 5151 | 2P | BG9NUC-LC39 |
| 5152 | RBD | BG9NUC-LC40 |
| 5153 | 2P | BG9NUC-LC41 |
| 5154 | 2P | BG9NUC-LC42 |
| 5155 | RBD | BG9NUC-LC43 |
| 5156 | 2P | BG9NUC-LC44 |
| 5157 | 2F | BG9NUC-LC45 |
| 5158 | RBD | BG9NUC-LC46 |
| 5159 | 2P | BG9NUC-LC47 |
| 5160 | 2P | BG9NUC-LC48 |
| 5161 | RBD | BG9NUC-LC49 |
| 5162 | 2P | BG9NUC-LC50 |
| 5163 | 2P | BG9NUC-LC51 |
| 5164 | RBD | BG9NUC-LC52 |
| 5165 | 2P | BG9NUC-LC53 |
| 5166 | RBD | BG9NUC-LC54 |
| 5167 | RBD | BG9NUC-LC55 |
| 5168 | 2P | BG9NUC-LC56 |
| 5169 | 2F | BG9NUC-LC57 |
| 5170 | RBD | BG9NUC-LC58 |
| 5171 | RBD | BG9NUC-LC59 |
| 5172 | RBD | BG9NUC-LC60 |
| 5173 | 2P | BG9NUC-LC61 |
| 5174 | 2P | BG9NUC-LC62 |
| 5175 | RBD | BG9NUC-LC63 |
| 5176 | 2P | BG9NUC-LC64 |
| 5177 | 2P | BG9NUC-LC65 |
| 5178 | 2P | BG9NUC-LC66 |
| 5179 | 2P | BG9NUC-LC67 |
| 5180 | 2P | BG9NUC-LC68 |
| 5181 | RBD | BG9NUC-LC69 |
| 5182 | 2F | BG9NUC-LC70 |
| 5183 | 2P | BG9NUC-LC71 |
| 5184 | RBD | BG9NUC-LC72 |
| 5185 | 2P | BG9NUC-LC73 |
| 5186 | 2P | BG9NUC-LC74 |
| 5187 | 2P | BG9NUC-LC75 |
| 5188 | RBD | BG9NUC-LC76 |
| 5189 | RBD | BG9NUC-LC77 |
| 5190 | RBD | BG9NUC-LC78 |
| 5191 | 2F | BG9NUC-LC79 |
| 5192 | 2P | BG9NUC-LC80 |
| 5193 | 2P | BG9NUC-LC81 |
| 5194 | 2P | BG9NUC-LC82 |
| 5195 | RBD | BG9NUC-LC83 |
| 5196 | RBD | BG9NUC-LC84 |
| 5197 | RBD | BG9NUC-LC85 |
| 5198 | RBD | BG9NUC-LC86 |
| 5199 | 2P | BG9NUC-LC87 |
| 5200 | 2P | BG9NUC-LC88 |
| 5201 | 2P | BG9NUC-LC89 |
| 5202 | 2P | BG9NUC-LC90 |
| 5203 | 2P | BG9NUC-LC91 |
| 5204 | RBD | BG9NUC-LC92 |
| 5205 | 2P | BG9NUC-LC93 |
| 5206 | 2P | BG9NUC-LC94 |
| 5207 | RBD | BG9NUC-LC95 |
| 5208 | 2P | BG9NUC-LC96 |
| 5209 | 2P | BG9NUC-LC97 |
| 5210 | 2P | BG9NUC-LC98 |
| 5211 | 2P | BG9NUC-LC99 |
| 5212 | RBD | BG9NUC-LC100 |
| 5213 | RBD | BG9NUC-LC101 |
| 5214 | RBD | BG9NUC-LC102 |
| 5215 | 2P | BG9NUC-LC103 |
| 5216 | RBD | BG9NUC-LC104 |
| 5217 | 2P | BG9NUC-LC105 |
| 5218 | 2P | BG9NUC-LC106 |
| 5219 | RBD | BG9NUC-LC107 |
| 5220 | RBD | BG9NUC-LC108 |
| 5221 | RBD | BG9NUC-LC109 |
| 5222 | 2P | BG9NUC-LC110 |
| 5223 | 2P | BG9NUC-LC111 |
| 5224 | 2P | BG9NUC-LC112 |
| 5225 | RBD | BG9NUC-LC113 |
| 5226 | 2P | BG9NUC-LC114 |
| 5227 | 2P | BG9NUC-LC115 |
| 5228 | RBD | BG9NUC-LC116 |
| 5229 | RBD | BG9NUC-LC117 |
| 5230 | RBD | BG9NUC-LC118 |
| 5231 | 2P | BG9NUC-LC119 |
| 5232 | 2P | BG9NUC-LC120 |
| 5233 | RBD | BG9NUC-LC121 |
| 5234 | RBD | BG9NUC-LC122 |
| 5235 | 2P | BG9NUC-LC123 |
| 5236 | 2P | BG9NUC-LC124 |
| 5237 | 2P | BG9NUC-LC125 |
| 5238 | 2P | BG9NUC-LC126 |
| 5239 | RBD | BG9NUC-LC127 |
| 5240 | 2P | BG9NUC-LC128 |
| 5241 | RBD | BG9NUC-LC129 |
| 5242 | RBD | BG9NUC-LC130 |
| 5243 | RBD | BG9NUC-LC131 |
| 5244 | RBD | BG9NUC-LC132 |
| 5245 | 2P | BG9NUC-LC133 |
| 5246 | RBD | BG9NUC-LC134 |
| 5247 | 2P | BG9NUC-LC135 |
| 5248 | RBD | BG9NUC-LC136 |
| 5249 | 2P | BG9NUC-LC137 |
| 5250 | RBD | BG9NUC-LC138 |
| 5251 | 2P | BG9NUC-LC139 |
| 5252 | 2P | BG9NUC-LC140 |
| 5253 | 2P | BG9NUC-LC141 |
| 5254 | 2P | BG9NUC-LC142 |
| 5255 | 2P | BG9NUC-LC143 |
| 5256 | 2P | BG9NUC-LC144 |
| 5257 | 2P | BG9NUC-LC145 |
| 5258 | 2P | BG9NUC-LC146 |
| 5259 | RBD | BG9NUC-LC147 |
| 5260 | RBD | BG9NUC-LC148 |
| 5261 | RBD | BG9NUC-LC149 |
| 5262 | RBD | BG9NUC-LC150 |
| 5263 | 2P | BG9NUC-LC151 |
| 5264 | 2P | BG9NUC-LC152 |
| 5265 | RBD | BG9NUC-LC153 |
| 5266 | 2P | BG9NUC-LC154 |
| 5267 | 2P | BG9NUC-LC155 |
| 5268 | 2P | BG9NUC-LC156 |
| 5269 | 2P | BG9NUC-LC157 |
| 5270 | RBD | BG9NUC-LC158 |
| 5271 | 2P | BG9NUC-LC159 |
| 5272 | 2P | BG9NUC-LC160 |
| 5273 | 2P | BG9NUC-LC161 |
| 5274 | RBD | BG9NUC-LC162 |
| 5275 | 2P | BG9NUC-LC163 |
| 5276 | 2P | BG9NUC-LC164 |
| 5277 | RBD | BG9NUC-LC165 |
| 5278 | 2P | BG9NUC-LC166 |
| 5279 | RBD | BG9NUC-LC167 |
| 5280 | RBD | BG9NUC-LC168 |
| 5281 | RBD | BG9NUC-LC169 |
| 5282 | RBD | BG9NUC-LC170 |
| 5283 | RBD | BG9NUC-LC171 |
| 5284 | 2P | BG9NUC-LC172 |
| 5285 | RBD | BG9NUC-LC173 |
| 5286 | 2P | BG9NUC-LC174 |
| 5287 | RBD | BG9NUC-LC175 |
| 5288 | RBD | BG9NUC-LC176 |
| 5289 | 2P | BG9NUC-LC177 |
| 5290 | RBD | BG9NUC-LC178 |
| 5291 | 2P | BG9NUC-LC179 |
| 5292 | 2P | BG9NUC-LC180 |
| 5293 | 2P | BG9NUC-LC181 |
| 5294 | RBD | BG9NUC-LC182 |
| 5295 | 2P | BG9NUC-LC183 |
| 5296 | RBD | BG9NUC-LC184 |
| 5297 | 2P | BG9NUC-LC185 |
| 5298 | RBD | BG9NUC-LC186 |
| 5299 | 2P | BG9NUC-LC187 |
| 5300 | 2P | BG9NUC-LC188 |
| 5301 | 2P | BG9NUC-LC189 |
| 5302 | 2P | BG9NUC-LC190 |
| 5303 | 2P | BG9NUC-LC191 |
| 5304 | RBD | BG9NUC-LC192 |
| 5305 | 2P | BG9NUC-LC193 |
| 5306 | 2P | BG9NUC-LC194 |
| 5307 | RBD | BG9NUC-LC195 |
| 5308 | RBD | BG9NUC-LC196 |
| 5309 | 2P | BG9NUC-LC197 |
| 5310 | RBD | BG9NUC-LC198 |
| 5311 | 2P | BG9NUC-LC199 |
| 5312 | 2P | BG9NUC-LC200 |
| 5313 | RBD | BG9NUC-LC201 |
| 5314 | 2P | BG9NUC-LC202 |
| 5315 | 2P | BG9NUC-LC203 |
| 5316 | 2P | BG9NUC-LC204 |
| 5317 | 2P | BG9NUC-LC205 |
| 5318 | RBD | BG9NUC-LC206 |
| 5319 | RBD | BG9NUC-LC207 |
| 5320 | 2P | BG9NUC-LC208 |
| 5321 | RBD | BG9NUC-LC209 |
| 5322 | RBD | BG9NUC-LC210 |
| 5323 | 2P | BG9NUC-LC211 |
| 5324 | RBD | BG9NUC-LC212 |
| 5325 | RBD | BG9NUC-LC213 |
| 5326 | 2P | BG9NUC-LC214 |
| 5327 | 2P | BG9NUC-LC215 |
| 5328 | RBD | BG9NUC-LC216 |
| 5329 | 2P | BG9NUC-LC217 |
| 5330 | 2P | BG9NUC-LC218 |
| 5331 | RBD | BG9NUC-LC219 |
| 5332 | RBD | BG9NUC-LC220 |
| 5333 | 2P | BG9NUC-LC221 |
| 5334 | RBD | BG9NUC-LC222 |
| 5335 | 2P | BG9NUC-LC223 |
| 5336 | 2P | BG9NUC-LC224 |
| 5337 | RBD | BG9NUC-LC225 |
| 5338 | 2P | BG9NUC-LC226 |
| 5339 | RBD | BG9NUC-LC227 |
| 5340 | RBD | BG9NUC-LC228 |
| 5341 | 2P | BG9NUC-LC229 |
| 5342 | 2P | BG9NUC-LC230 |
| 5343 | 2P | BG9NUC-LC231 |
| 5344 | RBD | BG9NUC-LC232 |
| 5345 | RBD | BG9NUC-LC233 |
| 5346 | RBD | BG9NUC-LC234 |
| 5347 | RBD | BG10NUC-HC1 |
| 5348 | RBD | BG10NUC-HC2 |
| 5349 | RBD | BG10NUC-HC3 |
| 5350 | RBD | BG10NUC-HC4 |
| 5351 | RBD | BG10NUC-HC5 |
| 5352 | RBD | BG10NUC-HC6 |
| 5353 | RBD | BG10NUC-HC7 |
| 5354 | RBD | BG10NUC-HC8 |
| 5355 | RBD | BG10NUC-HC9 |
| 5356 | RBD | BG10NUC-HC10 |
| 5357 | RBD | BG10NUC-HC11 |
| 5358 | RBD | BG10NUC-HC12 |
| 5359 | RBD | BG10NUC-HC13 |
| 5360 | RBD | BG10NUC-HC14 |
| 5361 | RBD | BG10NUC-HC15 |
| 5362 | RBD | BG10NUC-HC16 |
| 5363 | RBD | BG10NUC-HC17 |
| 5364 | RBD | BG10NUC-HC18 |
| 5365 | RBD | BG10NUC-HC19 |
| 5366 | RBD | BG10NUC-HC20 |
| 5367 | RBD | BG10NUC-HC21 |
| 5368 | RBD | BG10NUC-HC22 |
| 5369 | RBD | BG10NUC-HC23 |
| 5370 | RBD | BG10NUC-HC24 |
| 5371 | RBD | BG10NUC-HC25 |
| 5372 | RBD | BG10NUC-HC26 |
| 5373 | RBD | BG10NUC-HC27 |
| 5374 | 2P | BG10NUC-HC28 |
| 5375 | 2P | BG10NUC-HC29 |
| 5376 | 2P | BG10NUC-HC30 |
| 5377 | 2P | BG10NUC-HC31 |
| 5378 | 2P | BG10NUC-HC32 |
| 5379 | 2P | BG10NUC-HC33 |
| 5380 | 2P | BG10NUC-HC34 |
| 5381 | 2P | BG10NUC-HC35 |
| 5382 | 2P | BG10NUC-HC36 |
| 5383 | RBD | BG10NUC-HC37 |
| 5384 | RBD | BG10NUC-HC38 |
| 5385 | RBD | BG10NUC-HC39 |
| 5386 | RBD | BG10NUC-HC40 |
| 5387 | RBD | BG10NUC-HC41 |
| 5388 | RBD | BG10NUC-HC42 |
| 5389 | RBD | BG10NUC-HC43 |
| 5390 | RBD | BG10NUC-HC44 |
| 5391 | RBD | BG10NUC-HC45 |
| 5392 | RBD | BG10NUC-HC46 |
| 5393 | RBD | BG10NUC-HC47 |
| 5394 | RBD | BG10NUC-HC48 |
| 5395 | RBD | BG10NUC-HC49 |
| 5396 | RBD | BG10NUC-HC50 |
| 5397 | RBD | BG10NUC-HC51 |
| 5398 | RBD | BG10NUC-HC52 |
| 5399 | RBD | BG10NUC-HC53 |
| 5400 | RBD | BG10NUC-HC54 |
| 5401 | 2P | BG10NUC-HC55 |
| 5402 | RBD | BG10NUC-HC56 |
| 5403 | RBD | BG10NUC-HC57 |
| 5404 | 2P | BG10NUC-HC58 |
| 5405 | RBD | BG10NUC-HC59 |
| 5406 | RBD | BG10NUC-HC60 |
| 5407 | RBD | BG10NUC-HC61 |
| 5408 | RBD | BG10NUC-HC62 |
| 5409 | RBD | BG10NUC-HC63 |
| 5410 | RBD | BG10NUC-HC64 |
| 5411 | RBD | BG10NUC-HC65 |
| 5412 | RBD | BG10NUC-HC66 |
| 5413 | 2P | BG10NUC-HC67 |
| 5414 | 2P | BG10NUC-HC68 |
| 5415 | 2P | BG10NUC-HC69 |
| 5416 | RBD | BG10NUC-HC70 |
| 5417 | RBD | BG10NUC-HC71 |
| 5418 | RBD | BG10NUC-HC72 |
| 5419 | RBD | BG10NUC-HC73 |
| 5420 | 2P | BG10NUC-HC74 |
| 5421 | 2P | BG10NUC-HC75 |
| 5422 | 2P | BG10NUC-HC76 |
| 5423 | 2P | BG10NUC-HC77 |
| 5424 | RBD | BG10NUC-HC78 |
| 5425 | RBD | BG10NUC-HC79 |
| 5426 | RBD | BG10NUC-HC80 |
| 5427 | RBD | BG10NUC-HC81 |
| 5428 | 2P | BG10NUC-HC82 |
| 5429 | RBD | BG10NUC-HC83 |
| 5430 | RBD | BG10NUC-HC84 |
| 5431 | RBD | BG10NUC-HC85 |
| 5432 | RBD | BG10NUC-HC86 |
| 5433 | RBD | BG10NUC-HC87 |
| 5434 | 2P | BG10NUC-HC88 |
| 5435 | 2P | BG10NUC-HC89 |
| 5436 | RBD | BG10NUC-HC90 |
| 5437 | RBD | BG10NUC-HC91 |
| 5438 | 2P | BG10NUC-HC92 |
| 5439 | RBD | BG10NUC-HC93 |
| 5440 | RBD | BG10NUC-HC94 |
| 5441 | 2P | BG10NUC-HC95 |
| 5442 | 2P | BG10NUC-HC96 |
| 5443 | 2P | BG10NUC-HC97 |
| 5444 | 2P | BG10NUC-HC98 |
| 5445 | 2P | BG10NUC-HC99 |
| 5446 | RBD | BG10NUC-HC100 |
| 5447 | RBD | BG10NUC-HC101 |
| 5448 | 2P | BG10NUC-HC102 |
| 5449 | 2P | BG10NUC-HC103 |
| 5450 | RBD | BG10NUC-HC104 |
| 5451 | 2P | BG10NUC-HC105 |
| 5452 | 2P | BG10NUC-HC106 |
| 5453 | 2P | BG10NUC-HC107 |
| 5454 | 2P | BG10NUC-HC108 |
| 5455 | 2P | BG10NUC-HC109 |
| 5456 | 2P | BG10NUC-HC110 |
| 5457 | RBD | BG10NUC-HC111 |
| 5458 | 2P | BG10NUC-HC112 |
| 5459 | 2P | BG10NUC-HC113 |
| 5460 | 2P | BG10NUC-HC114 |
| 5461 | RBD | BG10NUC-HC115 |
| 5462 | 2P | BG10NUC-HC116 |
| 5463 | 2P | BG10NUC-HC117 |
| 5464 | 2P | BG10NUC-HC118 |
| 5465 | 2P | BG10NUC-HC119 |
| 5466 | 2P | BG10NUC-HC120 |
| 5467 | 2P | BG10NUC-HC121 |
| 5468 | 2P | BG10NUC-HC122 |
| 5469 | 2P | BG10NUC-HC123 |
| 5470 | 2P | BG10NUC-HC124 |
| 5471 | 2P | BG10NUC-HC125 |
| 5472 | 2P | BG10NUC-HC126 |
| 5473 | RBD | BG10NUC-HC127 |
| 5474 | RBD | BG10NUC-HC128 |
| 5475 | RBD | BG10NUC-HC129 |
| 5476 | 2P | BG10NUC-HC130 |
| 5477 | 2P | BG10NUC-HC131 |
| 5478 | 2P | BG10NUC-HC132 |
| 5479 | 2P | BG10NUC-HC133 |
| 5480 | 2P | BG10NUC-HC134 |
| 5481 | 2P | BG10NUC-HC135 |
| 5482 | 2P | BG10NUC-HC136 |
| 5483 | 2P | BG10NUC-HC137 |
| 5484 | RBD | BG10NUC-HC138 |
| 5485 | 2P | BG10NUC-HC139 |
| 5486 | 2P | BG10NUC-HC140 |
| 5487 | RBD | BG10NUC-HC141 |
| 5488 | RBD | BG10NUC-HC142 |
| 5489 | 2P | BG10NUC-HC143 |
| 5490 | 2P | BG10NUC-HC144 |
| 5491 | 2P | BG10NUC-HC145 |
| 5492 | 2P | BG10NUC-HC146 |
| 5493 | 2P | BG10NUC-HC147 |
| 5494 | 2P | BG10NUC-HC148 |
| 5495 | 2P | BG10NUC-HC149 |
| 5496 | 2P | BG10NUC-HC150 |
| 5497 | RBD | BG10NUC-HC151 |
| 5498 | RBD | BG10NUC-HC152 |
| 5499 | RBD | BG10NUC-HC153 |
| 5500 | RBD | BG10NUC-HC154 |
| 5501 | 2P | BG10NUC-HC155 |
| 5502 | 2P | BG10NUC-HC156 |
| 5503 | 2P | BG10NUC-HC157 |
| 5504 | RBD | BG10NUC-HC158 |
| 5505 | 2P | BG10NUC-HC159 |
| 5506 | RBD | BG10NUC-HC160 |
| 5507 | 2P | BG10NUC-HC161 |
| 5508 | RBD | BG10NUC-HC162 |
| 5509 | RBD | BG10NUC-HC163 |
| 5510 | RBD | BG10NUC-HC164 |
| 5511 | 2P | BG10NUC-HC165 |
| 5512 | 2P | BG10NUC-HC166 |
| 5513 | 2P | BG10NUC-HC167 |
| 5514 | RBD | BG10NUC-HC168 |
| 5515 | RBD | BG10NUC-HC169 |
| 5516 | RBD | BG10NUC-HC170 |
| 5517 | 2P | BG10NUC-HC171 |
| 5518 | 2P | BG10NUC-HC172 |
| 5519 | 2P | BG10NUC-HC173 |
| 5520 | 2P | BG10NUC-HC174 |
| 5521 | 2F | BG10NUC-HC175 |
| 5522 | 2P | BG10NUC-HC176 |
| 5523 | RBD | BG10NUC-HC177 |
| 5524 | 2P | BG10NUC-HC178 |
| 5525 | RBD | BG10NUC-HC179 |
| 5526 | 2P | BG10NUC-HC180 |
| 5527 | 2P | BG10NUC-HC181 |
| 5528 | RBD | BG10NUC-HC182 |
| 5529 | 2P | BG10NUC-HC183 |
| 5530 | 2P | BG10NUC-HC184 |
| 5531 | 2P | BG10NUC-HC185 |
| 5532 | 2P | BG10NUC-HC186 |
| 5533 | 2P | BG10NUC-HC187 |
| 5534 | 2P | BG10NUC-HC188 |
| 5535 | 2P | BG10NUC-HC189 |
| 5536 | 2P | BG10NUC-HC190 |
| 5537 | 2P | BG10NUC-HC191 |
| 5538 | RBD | BG10NUC-HC192 |
| 5539 | 2P | BG10NUC-HC193 |
| 5540 | RBD | BG10NUC-HC194 |
| 5541 | 2P | BG10NUC-HC195 |
| 5542 | RBD | BG10NUC-HC196 |
| 5543 | 2P | BG10NUC-HC197 |
| 5544 | 2P | BG10NUC-HC198 |
| 5545 | RBD | BG10NUC-HC199 |
| 5546 | 2P | BG10NUC-HC200 |
| 5547 | RBD | BG10NUC-HC201 |
| 5548 | 2P | BG10NUC-HC202 |
| 5549 | RBD | BG10NUC-HC203 |
| 5550 | 2P | BG10NUC-HC204 |
| 5551 | RBD | BG10NUC-HC205 |
| 5552 | 2P | BG10NUC-HC206 |
| 5553 | RBD | BG10NUC-HC207 |
| 5554 | RBD | BG10NUC-HC208 |
| 5555 | 2P | BG10NUC-HC209 |
| 5556 | 2P | BG10NUC-HC210 |
| 5557 | 2P | BG10NUC-HC211 |
| 5558 | 2P | BG10NUC-HC212 |
| 5559 | 2P | BG10NUC-HC213 |
| 5560 | 2P | BG10NUC-HC214 |
| 5561 | 2P | BG10NUC-HC215 |
| 5562 | 2P | BG10NUC-HC216 |
| 5563 | 2P | BG10NUC-HC217 |
| 5564 | RBD | BG10NUC-HC218 |
| 5565 | 2P | BG10NUC-HC219 |
| 5566 | 2P | BG10NUC-HC220 |
| 5567 | 2F | BG10NUC-HC221 |
| 5568 | 2F | BG10NUC-HC222 |
| 5569 | RBD | BG10NUC-HC223 |
| 5570 | RBD | BG10NUC-HC224 |
| 5571 | RBD | BG10NUC-HC225 |
| 5572 | 2P | BG10NUC-HC226 |
| 5573 | 2F | BG10NUC-HC227 |
| 5574 | 2P | BG10NUC-HC228 |
| 5575 | 2P | BG10NUC-HC229 |
| 5576 | 2F | BG10NUC-HC230 |
| 5577 | 2P | BG10NUC-HC231 |
| 5578 | RBD | BG10NUC-HC232 |
| 5579 | RBD | BG10NUC-HC233 |
| 5580 | RBD | BG10NUC-HC234 |
| 5581 | RBD | BG10NUC-HC235 |
| 5582 | 2P | BG10NUC-HC236 |
| 5583 | RBD | BG10NUC-HC237 |
| 5584 | RBD | BG10NUC-HC238 |
| 5585 | 2P | BG10NUC-HC239 |
| 5586 | 2P | BG10NUC-HC240 |
| 5587 | 2P | BG10NUC-HC241 |
| 5588 | 2P | BG10NUC-HC242 |
| 5589 | RBD | BG10NUC-HC243 |
| 5590 | 2P | BG10NUC-HC244 |
| 5591 | RBD | BG10NUC-HC245 |
| 5592 | 2P | BG10NUC-HC246 |
| 5593 | 2P | BG10NUC-HC247 |
| 5594 | 2P | BG10NUC-HC248 |
| 5595 | 2P | BG10NUC-HC249 |
| 5596 | 2P | BG10NUC-HC250 |
| 5597 | 2P | BG10NUC-HC251 |
| 5598 | 2F | BG10NUC-HC252 |
| 5599 | 2P | BG10NUC-HC253 |
| 5600 | 2P | BG10NUC-HC254 |
| 5601 | 2P | BG10NUC-HC255 |
| 5602 | 2P | BG10NUC-HC256 |
| 5603 | 2P | BG10NUC-HC257 |
| 5604 | 2P | BG10NUC-HC258 |
| 5605 | RBD | BG10NUC-HC259 |
| 5606 | RBD | BG10NUC-HC260 |
| 5607 | 2P | BG10NUC-HC261 |
| 5608 | RBD | BG10NUC-HC262 |
| 5609 | 2P | BG10NUC-HC263 |
| 5610 | 2P | BG10NUC-HC264 |
| 5611 | RBD | BG10NUC-HC265 |
| 5612 | RBD | BG10NUC-HC266 |
| 5613 | RBD | BG10NUC-HC267 |
| 5614 | RBD | BG10NUC-HC268 |
| 5615 | 2P | BG10NUC-HC269 |
| 5616 | 2P | BG10NUC-HC270 |
| 5617 | RBD | BG10NUC-HC271 |
| 5618 | 2P | BG10NUC-HC272 |
| 5619 | 2P | BG10NUC-HC273 |
| 5620 | RBD | BG10NUC-HC274 |
| 5621 | 2P | BG10NUC-HC275 |
| 5622 | RBD | BG10NUC-HC276 |
| 5623 | RBD | BG10NUC-HC277 |
| 5624 | RBD | BG10NUC-HC278 |
| 5625 | 2P | BG10NUC-HC279 |
| 5626 | 2P | BG10NUC-HC280 |
| 5627 | 2P | BG10NUC-HC281 |
| 5628 | 2F | BG10NUC-HC282 |
| 5629 | 2F | BG10NUC-HC283 |
| 5630 | 2P | BG10NUC-HC284 |
| 5631 | 2P | BG10NUC-HC285 |
| 5632 | 2P | BG10NUC-HC286 |
| 5633 | RBD | BG10NUC-HC287 |
| 5634 | RBD | BG10NUC-HC288 |
| 5635 | RBD | BG10NUC-HC289 |
| 5636 | 2P | BG10NUC-HC290 |
| 5637 | 2P | BG10NUC-HC291 |
| 5638 | RBD | BG10NUC-HC292 |
| 5639 | 2P | BG10NUC-HC293 |
| 5640 | RBD | BG10NUC-HC294 |
| 5641 | RBD | BG10NUC-HC295 |
| 5642 | RBD | BG10NUC-HC296 |
| 5643 | RBD | BG10NUC-HC297 |
| 5644 | 2P | BG10NUC-HC298 |
| 5645 | RBD | BG10NUC-HC299 |
| 5646 | RBD | BG10NUC-HC300 |
| 5647 | RBD | BG10NUC-HC301 |
| 5648 | RBD | BG10NUC-HC302 |
| 5649 | RBD | BG10NUC-HC303 |
| 5650 | RBD | BG10NUC-HC304 |
| 5651 | 2P | BG10NUC-HC305 |
| 5652 | RBD | BG10NUC-HC306 |
| 5653 | RBD | BG10NUC-HC307 |
| 5654 | 2P | BG10NUC-HC308 |
| 5655 | 2P | BG10NUC-HC309 |
| 5656 | RBD | BG10NUC-HC310 |
| 5657 | 2P | BG10NUC-HC311 |
| 5658 | 2P | BG10NUC-HC312 |
| 5659 | 2P | BG10NUC-HC313 |
| 5660 | 2P | BG10NUC-HC314 |
| 5661 | 2P | BG10NUC-HC315 |
| 5662 | 2P | BG10NUC-HC316 |
| 5663 | 2F | BG10NUC-HC317 |
| 5664 | RBD | BG10NUC-HC318 |
| 5665 | 2P | BG10NUC-HC319 |
| 5666 | 2P | BG10NUC-HC320 |
| 5667 | 2P | BG10NUC-HC321 |
| 5668 | 2P | BG10NUC-HC322 |
| 5669 | 2P | BG10NUC-HC323 |
| 5670 | RBD | BG10NUC-HC324 |
| 5671 | RBD | BG10NUC-HC325 |
| 5672 | 2P | BG10NUC-HC326 |
| 5673 | 2P | BG10NUC-HC327 |
| 5674 | 2P | BG10NUC-HC328 |
| 5675 | 2P | BG10NUC-HC329 |
| 5676 | RBD | BG10NUC-HC330 |
| 5677 | 2P | BG10NUC-HC331 |
| 5678 | RBD | BG10NUC-HC332 |
| 5679 | RBD | BG10NUC-HC333 |
| 5680 | RBD | BG10NUC-HC334 |
| 5681 | 2P | BG10NUC-HC335 |
| 5682 | 2P | BG10NUC-HC336 |
| 5683 | 2P | BG10NUC-HC337 |
| 5684 | RBD | BG10NUC-HC338 |
| 5685 | 2P | BG10NUC-HC339 |
| 5686 | 2P | BG10NUC-HC340 |
| 5687 | 2P | BG10NUC-HC341 |
| 5688 | 2P | BG10NUC-HC342 |
| 5689 | RBD | BG10NUC-HC343 |
| 5690 | 2P | BG10NUC-HC344 |
| 5691 | RBD | BG10NUC-HC345 |
| 5692 | 2P | BG10NUC-HC346 |
| 5693 | 2P | BG10NUC-HC347 |
| 5694 | 2P | BG10NUC-HC348 |
| 5695 | 2P | BG10NUC-HC349 |
| 5696 | 2P | BG10NUC-HC350 |
| 5697 | 2P | BG10NUC-HC351 |
| 5698 | RBD | BG10NUC-HC352 |
| 5699 | 2P | BG10NUC-HC353 |
| 5700 | 2P | BG10NUC-HC354 |
| 5701 | 2F | BG10NUC-HC355 |
| 5702 | RBD | BG10NUC-HC356 |
| 5703 | RBD | BG10NUC-HC357 |
| 5704 | 2P | BG10NUC-HC358 |
| 5705 | RBD | BG10NUC-HC359 |
| 5706 | RBD | BG10NUC-HC360 |
| 5707 | 2P | BG10NUC-HC361 |
| 5708 | 2P | BG10NUC-HC362 |
| 5709 | 2P | BG10NUC-HC363 |
| 5710 | RBD | BG10NUC-HC364 |
| 5711 | 2P | BG10NUC-HC365 |
| 5712 | 2P | BG10NUC-HC366 |
| 5713 | RBD | BG10NUC-HC367 |
| 5714 | RBD | BG10NUC-HC368 |
| 5715 | 2P | BG10NUC-HC369 |
| 5716 | 2P | BG10NUC-HC370 |
| 5717 | 2P | BG10NUC-HC371 |
| 5718 | RBD | BG10NUC-HC372 |
| 5719 | RBD | BG10NUC-HC373 |
| 5720 | RBD | BG10NUC-HC374 |
| 5721 | 2P | BG10NUC-HC375 |
| 5722 | 2F | BG10NUC-HC376 |
| 5723 | 2P | BG10NUC-HC377 |
| 5724 | 2P | BG10NUC-HC378 |
| 5725 | 2P | BG10NUC-HC379 |
| 5726 | 2P | BG10NUC-HC380 |
| 5727 | 2P | BG10NUC-HC381 |
| 5728 | RBD | BG10NUC-HC382 |
| 5729 | RBD | BG10NUC-HC383 |
| 5730 | RBD | BG10NUC-HC384 |
| 5731 | RBD | BG10NUC-HC385 |
| 5732 | RBD | BG10NUC-HC386 |
| 5733 | 2P | BG10NUC-HC387 |
| 5734 | 2F | BG10NUC-HC388 |
| 5735 | RBD | BG10NUC-HC389 |
| 5736 | 2P | BG10NUC-HC390 |
| 5737 | 2P | BG10NUC-HC391 |
| 5738 | 2P | BG10NUC-HC392 |
| 5739 | 2P | BG10NUC-HC393 |
| 5740 | 2P | BG10NUC-HC394 |
| 5741 | 2P | BG10NUC-HC395 |
| 5742 | 2P | BG10NUC-HC396 |
| 5743 | RBD | BG10NUC-HC397 |
| 5744 | 2P | BG10NUC-HC398 |
| 5745 | 2P | BG10NUC-HC399 |
| 5746 | RBD | BG10NUC-HC400 |
| 5747 | 2P | BG10NUC-HC401 |
| 5748 | 2P | BG10NUC-HC402 |
| 5749 | 2P | BG10NUC-HC403 |
| 5750 | RBD | BG10NUC-HC404 |
| 5751 | RBD | BG10NUC-HC405 |
| 5752 | 2P | BG10NUC-HC406 |
| 5753 | 2P | BG10NUC-HC407 |
| 5754 | 2P | BG10NUC-HC408 |
| 5755 | 2P | BG10NUC-HC409 |
| 5756 | RBD | BG10NUC-HC410 |
| 5757 | 2P | BG10NUC-HC411 |
| 5758 | 2P | BG10NUC-HC412 |
| 5759 | 2P | BG10NUC-HC413 |
| 5760 | RBD | BG10NUC-HC414 |
| 5761 | 2P | BG10NUC-HC415 |
| 5762 | RBD | BG10NUC-HC416 |
| 5763 | 2P | BG10NUC-HC417 |
| 5764 | RBD | BG10NUC-HC418 |
| 5765 | RBD | BG10NUC-HC419 |
| 5766 | RBD | BG10NUC-HC420 |
| 5767 | 2P | BG10NUC-HC421 |
| 5768 | RBD | BG10NUC-HC422 |
| 5769 | RBD | BG10NUC-HC423 |
| 5770 | RBD | BG10NUC-HC424 |
| 5771 | RBD | BG10NUC-HC425 |
| 5772 | 2P | BG10NUC-HC426 |
| 5773 | RBD | BG10NUC-HC427 |
| 5774 | RBD | BG10NUC-LC1 |
| 5775 | RBD | BG10NUC-LC2 |
| 5776 | RBD | BG10NUC-LC3 |
| 5777 | RBD | BG10NUC-LC4 |
| 5778 | RBD | BG10NUC-LC5 |
| 5779 | RBD | BG10NUC-LC6 |
| 5780 | RBD | BG10NUC-LC7 |
| 5781 | RBD | BG10NUC-LC8 |
| 5782 | RBD | BG10NUC-LC9 |
| 5783 | RBD | BG10NUC-LC10 |
| 5784 | RBD | BG10NUC-LC11 |
| 5785 | RBD | BG10NUC-LC12 |
| 5786 | RBD | BG10NUC-LC13 |
| 5787 | RBD | BG10NUC-LC14 |
| 5788 | RBD | BG10NUC-LC15 |
| 5789 | RBD | BG10NUC-LC16 |
| 5790 | RBD | BG10NUC-LC17 |
| 5791 | RBD | BG10NUC-LC18 |
| 5792 | RBD | BG10NUC-LC19 |
| 5793 | RBD | BG10NUC-LC20 |
| 5794 | RBD | BG10NUC-LC21 |
| 5795 | RBD | BG10NUC-LC22 |
| 5796 | RBD | BG10NUC-LC23 |
| 5797 | RBD | BG10NUC-LC24 |
| 5798 | RBD | BG10NUC-LC25 |
| 5799 | RBD | BG10NUC-LC26 |
| 5800 | RBD | BG10NUC-LC27 |
| 5801 | 2P | BG10NUC-LC28 |
| 5802 | 2P | BG10NUC-LC29 |
| 5803 | 2P | BG10NUC-LC30 |
| 5804 | 2P | BG10NUC-LC31 |
| 5805 | 2P | BG10NUC-LC32 |
| 5806 | 2P | BG10NUC-LC33 |
| 5807 | 2P | BG10NUC-LC34 |
| 5808 | 2P | BG10NUC-LC35 |
| 5809 | 2P | BG10NUC-LC36 |
| 5810 | RBD | BG10NUC-LC37 |
| 5811 | RBD | BG10NUC-LC38 |
| 5812 | RBD | BG10NUC-LC39 |
| 5813 | RBD | BG10NUC-LC40 |
| 5814 | RBD | BG10NUC-LC41 |
| 5815 | RBD | BG10NUC-LC42 |
| 5816 | RBD | BG10NUC-LC43 |
| 5817 | RBD | BG10NUC-LC44 |
| 5818 | RBD | BG10NUC-LC45 |
| 5819 | RBD | BG10NUC-LC46 |
| 5820 | RBD | BG10NUC-LC47 |
| 5821 | RBD | BG10NUC-LC48 |
| 5822 | RBD | BG10NUC-LC49 |
| 5823 | RBD | BG10NUC-LC50 |
| 5824 | RBD | BG10NUC-LC51 |
| 5825 | RBD | BG10NUC-LC52 |
| 5826 | RBD | BG10NUC-LC53 |
| 5827 | RBD | BG10NUC-LC54 |
| 5828 | 2P | BG10NUC-LC55 |
| 5829 | RBD | BG10NUC-LC56 |
| 5830 | RBD | BG10NUC-LC57 |
| 5831 | 2P | BG10NUC-LC58 |
| 5832 | RBD | BG10NUC-LC59 |
| 5833 | RBD | BG10NUC-LC60 |
| 5834 | RBD | BG10NUC-LC61 |
| 5835 | RBD | BG10NUC-LC62 |
| 5836 | RBD | BG10NUC-LC63 |
| 5837 | RBD | BG10NUC-LC64 |
| 5838 | RBD | BG10NUC-LC65 |
| 5839 | RBD | BG10NUC-LC66 |
| 5840 | 2P | BG10NUC-LC67 |
| 5841 | 2F | BG10NUC-LC68 |
| 5842 | 2P | BG10NUC-LC69 |
| 5843 | RBD | BG10NUC-LC70 |
| 5844 | RBD | BG10NUC-LC71 |
| 5845 | RBD | BG10NUC-LC72 |
| 5846 | RBD | BG10NUC-LC73 |
| 5847 | 2P | BG10NUC-LC74 |
| 5848 | 2P | BG10NUC-LC75 |
| 5849 | 2P | BG10NUC-LC76 |
| 5850 | 2P | BG10NUC-LC77 |
| 5851 | RBD | BG10NUC-LC78 |
| 5852 | RBD | BG10NUC-LC79 |
| 5853 | RBD | BG10NUC-LC80 |
| 5854 | RBD | BG10NUC-LC81 |
| 5855 | 2P | BG10NUC-LC82 |
| 5856 | RBD | BG10NUC-LC83 |
| 5857 | RBD | BG10NUC-LC84 |
| 5858 | RBD | BG10NUC-LC85 |
| 5859 | RBD | BG10NUC-LC86 |
| 5860 | RBD | BG10NUC-LC87 |
| 5861 | 2P | BG10NUC-LC88 |
| 5862 | 2P | BG10NUC-LC89 |
| 5863 | RBD | BG10NUC-LC90 |
| 5864 | RBD | BG10NUC-LC91 |
| 5865 | 2P | BG10NUC-LC92 |
| 5866 | RBD | BG10NUC-LC93 |
| 5867 | RBD | BG10NUC-LC94 |
| 5868 | 2P | BG10NUC-LC95 |
| 5869 | 2P | BG10NUC-LC96 |
| 5870 | 2P | BG10NUC-LC97 |
| 5871 | 2P | BG10NUC-LC98 |
| 5872 | 2P | BG10NUC-LC99 |
| 5873 | RBD | BG10NUC-LC100 |
| 5874 | RBD | BG10NUC-LC101 |
| 5875 | 2P | BG10NUC-LC102 |
| 5876 | 2P | BG10NUC-LC103 |
| 5877 | RBD | BG10NUC-LC104 |
| 5878 | 2P | BG10NUC-LC105 |
| 5879 | 2P | BG10NUC-LC106 |
| 5880 | 2P | BG10NUC-LC107 |
| 5881 | 2P | BG10NUC-LC108 |
| 5882 | 2P | BG10NUC-LC109 |
| 5883 | 2P | BG10NUC-LC110 |
| 5884 | RBD | BG10NUC-LC111 |
| 5885 | 2P | BG10NUC-LC112 |
| 5886 | 2P | BG10NUC-LC113 |
| 5887 | 2P | BG10NUC-LC114 |
| 5888 | RBD | BG10NUC-LC115 |
| 5889 | 2P | BG10NUC-LC116 |
| 5890 | 2P | BG10NUC-LC117 |
| 5891 | 2P | BG10NUC-LC118 |
| 5892 | 2P | BG10NUC-LC119 |
| 5893 | 2P | BG10NUC-LC120 |
| 5894 | 2P | BG10NUC-LC121 |
| 5895 | 2P | BG10NUC-LC122 |
| 5896 | 2P | BG10NUC-LC123 |
| 5897 | 2P | BG10NUC-LC124 |
| 5898 | 2P | BG10NUC-LC125 |
| 5899 | 2P | BG10NUC-LC126 |
| 5900 | RBD | BG10NUC-LC127 |
| 5901 | RBD | BG10NUC-LC128 |
| 5902 | RBD | BG10NUC-LC129 |
| 5903 | 2P | BG10NUC-LC130 |
| 5904 | 2P | BG10NUC-LC131 |
| 5905 | 2P | BG10NUC-LC132 |
| 5906 | 2P | BG10NUC-LC133 |
| 5907 | 2P | BG10NUC-LC134 |
| 5908 | 2P | BG10NUC-LC135 |
| 5909 | 2P | BG10NUC-LC136 |
| 5910 | 2P | BG10NUC-LC137 |
| 5911 | RBD | BG10NUC-LC138 |
| 5912 | 2P | BG10NUC-LC139 |
| 5913 | 2P | BG10NUC-LC140 |
| 5914 | RBD | BG10NUC-LC141 |
| 5915 | RBD | BG10NUC-LC142 |
| 5916 | 2F | BG10NUC-LC143 |
| 5917 | 2P | BG10NUC-LC144 |
| 5918 | 2P | BG10NUC-LC145 |
| 5919 | 2P | BG10NUC-LC146 |
| 5920 | 2P | BG10NUC-LC147 |
| 5921 | 2P | BG10NUC-LC148 |
| 5922 | 2P | BG10NUC-LC149 |
| 5923 | 2P | BG10NUC-LC150 |
| 5924 | RBD | BG10NUC-LC151 |
| 5925 | RBD | BG10NUC-LC152 |
| 5926 | RBD | BG10NUC-LC153 |
| 5927 | RBD | BG10NUC-LC154 |
| 5928 | 2P | BG10NUC-LC155 |
| 5929 | 2P | BG10NUC-LC156 |
| 5930 | 2P | BG10NUC-LC157 |
| 5931 | RBD | BG10NUC-LC158 |
| 5932 | 2P | BG10NUC-LC159 |
| 5933 | RBD | BG10NUC-LC160 |
| 5934 | 2P | BG10NUC-LC161 |
| 5935 | RBD | BG10NUC-LC162 |
| 5936 | RBD | BG10NUC-LC163 |
| 5937 | RBD | BG10NUC-LC164 |
| 5938 | 2P | BG10NUC-LC165 |
| 5939 | 2P | BG10NUC-LC166 |
| 5940 | 2P | BG10NUC-LC167 |
| 5941 | RBD | BG10NUC-LC168 |
| 5942 | RBD | BG10NUC-LC169 |
| 5943 | RBD | BG10NUC-LC170 |
| 5944 | 2P | BG10NUC-LC171 |
| 5945 | 2P | BG10NUC-LC172 |
| 5946 | 2P | BG10NUC-LC173 |
| 5947 | 2P | BG10NUC-LC174 |
| 5948 | 2P | BG10NUC-LC175 |
| 5949 | 2P | BG10NUC-LC176 |
| 5950 | RBD | BG10NUC-LC177 |
| 5951 | 2P | BG10NUC-LC178 |
| 5952 | RBD | BG10NUC-LC179 |
| 5953 | 2P | BG10NUC-LC180 |
| 5954 | 2P | BG10NUC-LC181 |
| 5955 | RBD | BG10NUC-LC182 |
| 5956 | 2P | BG10NUC-LC183 |
| 5957 | 2P | BG10NUC-LC184 |
| 5958 | 2P | BG10NUC-LC185 |
| 5959 | 2P | BG10NUC-LC186 |
| 5960 | 2P | BG10NUC-LC187 |
| 5961 | 2P | BG10NUC-LC188 |
| 5962 | 2P | BG10NUC-LC189 |
| 5963 | 2P | BG10NUC-LC190 |
| 5964 | 2P | BG10NUC-LC191 |
| 5965 | RBD | BG10NUC-LC192 |
| 5966 | 2P | BG10NUC-LC193 |
| 5967 | RBD | BG10NUC-LC194 |
| 5968 | 2P | BG10NUC-LC195 |
| 5969 | RBD | BG10NUC-LC196 |
| 5970 | 2P | BG10NUC-LC197 |
| 5971 | 2P | BG10NUC-LC198 |
| 5972 | RBD | BG10NUC-LC199 |
| 5973 | 2P | BG10NUC-LC200 |
| 5974 | RBD | BG10NUC-LC201 |
| 5975 | 2P | BG10NUC-LC202 |
| 5976 | RBD | BG10NUC-LC203 |
| 5977 | 2P | BG10NUC-LC204 |
| 5978 | RBD | BG10NUC-LC205 |
| 5979 | 2P | BG10NUC-LC206 |
| 5980 | RBD | BG10NUC-LC207 |
| 5981 | RBD | BG10NUC-LC208 |
| 5982 | 2P | BG10NUC-LC209 |
| 5983 | 2P | BG10NUC-LC210 |
| 5984 | 2P | BG10NUC-LC211 |
| 5985 | 2P | BG10NUC-LC212 |
| 5986 | 2P | BG10NUC-LC213 |
| 5987 | 2P | BG10NUC-LC214 |
| 5988 | 2P | BG10NUC-LC215 |
| 5989 | 2P | BG10NUC-LC216 |
| 5990 | 2P | BG10NUC-LC217 |
| 5991 | RBD | BG10NUC-LC218 |
| 5992 | 2P | BG10NUC-LC219 |
| 5993 | 2P | BG10NUC-LC220 |
| 5994 | 2P | BG10NUC-LC221 |
| 5995 | 2P | BG10NUC-LC222 |
| 5996 | RBD | BG10NUC-LC223 |
| 5997 | RBD | BG10NUC-LC224 |
| 5998 | RBD | BG10NUC-LC225 |
| 5999 | 2P | BG10NUC-LC226 |
| 6000 | 2P | BG10NUC-LC227 |
| 6001 | 2P | BG10NUC-LC228 |
| 6002 | 2P | BG10NUC-LC229 |
| 6003 | 2P | BG10NUC-LC230 |
| 6004 | 2P | BG10NUC-LC231 |
| 6005 | RBD | BG10NUC-LC232 |
| 6006 | RBD | BG10NUC-LC233 |
| 6007 | RBD | BG10NUC-LC234 |
| 6008 | RBD | BG10NUC-LC235 |
| 6009 | 2P | BG10NUC-LC236 |
| 6010 | RBD | BG10NUC-LC237 |
| 6011 | RBD | BG10NUC-LC238 |
| 6012 | 2P | BG10NUC-LC239 |
| 6013 | 2P | BG10NUC-LC240 |
| 6014 | 2P | BG10NUC-LC241 |
| 6015 | 2P | BG10NUC-LC242 |
| 6016 | RBD | BG10NUC-LC243 |
| 6017 | 2P | BG10NUC-LC244 |
| 6018 | RBD | BG10NUC-LC245 |
| 6019 | 2P | BG10NUC-LC246 |
| 6020 | 2P | BG10NUC-LC247 |
| 6021 | 2P | BG10NUC-LC248 |
| 6022 | 2P | BG10NUC-LC249 |
| 6023 | 2P | BG10NUC-LC250 |
| 6024 | 2P | BG10NUC-LC251 |
| 6025 | 2P | BG10NUC-LC252 |
| 6026 | 2P | BG10NUC-LC253 |
| 6027 | 2P | BG10NUC-LC254 |
| 6028 | 2P | BG10NUC-LC255 |
| 6029 | 2P | BG10NUC-LC256 |
| 6030 | 2P | BG10NUC-LC257 |
| 6031 | 2P | BG10NUC-LC258 |
| 6032 | RBD | BG10NUC-LC259 |
| 6033 | RBD | BG10NUC-LC260 |
| 6034 | 2P | BG10NUC-LC261 |
| 6035 | RBD | BG10NUC-LC262 |
| 6036 | 2P | BG10NUC-LC263 |
| 6037 | 2P | BG10NUC-LC264 |
| 6038 | RBD | BG10NUC-LC265 |
| 6039 | RBD | BG10NUC-LC266 |
| 6040 | RBD | BG10NUC-LC267 |
| 6041 | RBD | BG10NUC-LC268 |
| 6042 | 2P | BG10NUC-LC269 |
| 6043 | 2P | BG10NUC-LC270 |
| 6044 | RBD | BG10NUC-LC271 |
| 6045 | 2P | BG10NUC-LC272 |
| 6046 | 2P | BG10NUC-LC273 |
| 6047 | RBD | BG10NUC-LC274 |
| 6048 | 2P | BG10NUC-LC275 |
| 6049 | RBD | BG10NUC-LC276 |
| 6050 | RBD | BG10NUC-LC277 |
| 6051 | RBD | BG10NUC-LC278 |
| 6052 | 2P | BG10NUC-LC279 |
| 6053 | 2P | BG10NUC-LC280 |
| 6054 | 2P | BG10NUC-LC281 |
| 6055 | 2P | BG10NUC-LC282 |
| 6056 | 2P | BG10NUC-LC283 |
| 6057 | 2P | BG10NUC-LC284 |
| 6058 | 2P | BG10NUC-LC285 |
| 6059 | 2P | BG10NUC-LC286 |
| 6060 | RBD | BG10NUC-LC287 |
| 6061 | RBD | BG10NUC-LC288 |
| 6062 | RBD | BG10NUC-LC289 |
| 6063 | 2P | BG10NUC-LC290 |
| 6064 | 2P | BG10NUC-LC291 |
| 6065 | RBD | BG10NUC-LC292 |
| 6066 | 2P | BG10NUC-LC293 |
| 6067 | RBD | BG10NUC-LC294 |
| 6068 | RBD | BG10NUC-LC295 |
| 6069 | RBD | BG10NUC-LC296 |
| 6070 | RBD | BG10NUC-LC297 |
| 6071 | 2P | BG10NUC-LC298 |
| 6072 | RBD | BG10NUC-LC299 |
| 6073 | RBD | BG10NUC-LC300 |
| 6074 | RBD | BG10NUC-LC301 |
| 6075 | RBD | BG10NUC-LC302 |
| 6076 | RBD | BG10NUC-LC303 |
| 6077 | RBD | BG10NUC-LC304 |
| 6078 | 2P | BG10NUC-LC305 |
| 6079 | RBD | BG10NUC-LC306 |
| 6080 | RBD | BG10NUC-LC307 |
| 6081 | 2P | BG10NUC-LC308 |
| 6082 | 2P | BG10NUC-LC309 |
| 6083 | RBD | BG10NUC-LC310 |
| 6084 | 2P | BG10NUC-LC311 |
| 6085 | 2P | BG10NUC-LC312 |
| 6086 | 2P | BG10NUC-LC313 |
| 6087 | 2P | BG10NUC-LC314 |
| 6088 | 2P | BG10NUC-LC315 |
| 6089 | 2P | BG10NUC-LC316 |
| 6090 | 2P | BG10NUC-LC317 |
| 6091 | RBD | BG10NUC-LC318 |
| 6092 | 2P | BG10NUC-LC319 |
| 6093 | 2P | BG10NUC-LC320 |
| 6094 | 2P | BG10NUC-LC321 |
| 6095 | 2P | BG10NUC-LC322 |
| 6096 | 2P | BG10NUC-LC323 |
| 6097 | RBD | BG10NUC-LC324 |
| 6098 | RBD | BG10NUC-LC325 |
| 6099 | 2P | BG10NUC-LC326 |
| 6100 | 2P | BG10NUC-LC327 |
| 6101 | 2P | BG10NUC-LC328 |
| 6102 | 2P | BG10NUC-LC329 |
| 6103 | RBD | BG10NUC-LC330 |
| 6104 | 2P | BG10NUC-LC331 |
| 6105 | RBD | BG10NUC-LC332 |
| 6106 | RBD | BG10NUC-LC333 |
| 6107 | RBD | BG10NUC-LC334 |
| 6108 | 2P | BG10NUC-LC335 |
| 6109 | 2P | BG10NUC-LC336 |
| 6110 | 2P | BG10NUC-LC337 |
| 6111 | RBD | BG10NUC-LC338 |
| 6112 | 2P | BG10NUC-LC339 |
| 6113 | 2P | BG10NUC-LC340 |
| 6114 | 2P | BG10NUC-LC341 |
| 6115 | 2P | BG10NUC-LC342 |
| 6116 | RBD | BG10NUC-LC343 |
| 6117 | 2P | BG10NUC-LC344 |
| 6118 | RBD | BG10NUC-LC345 |
| 6119 | 2P | BG10NUC-LC346 |
| 6120 | 2P | BG10NUC-LC347 |
| 6121 | 2P | BG10NUC-LC348 |
| 6122 | 2P | BG10NUC-LC349 |
| 6123 | 2P | BG10NUC-LC350 |
| 6124 | 2P | BG10NUC-LC351 |
| 6125 | RBD | BG10NUC-LC352 |
| 6126 | 2P | BG10NUC-LC353 |
| 6127 | 2P | BG10NUC-LC354 |
| 6128 | 2P | BG10NUC-LC355 |
| 6129 | RBD | BG10NUC-LC356 |
| 6130 | RBD | BG10NUC-LC357 |
| 6131 | 2P | BG10NUC-LC358 |
| 6132 | RBD | BG10NUC-LC359 |
| 6133 | RBD | BG10NUC-LC360 |
| 6134 | 2P | BG10NUC-LC361 |
| 6135 | 2P | BG10NUC-LC362 |
| 6136 | 2P | BG10NUC-LC363 |
| 6137 | RBD | BG10NUC-LC364 |
| 6138 | 2P | BG10NUC-LC365 |
| 6139 | 2P | BG10NUC-LC366 |
| 6140 | RBD | BG10NUC-LC367 |
| 6141 | RBD | BG10NUC-LC368 |
| 6142 | 2P | BG10NUC-LC369 |
| 6143 | 2P | BG10NUC-LC370 |
| 6144 | 2F | BG10NUC-LC371 |
| 6145 | RBD | BG10NUC-LC372 |
| 6146 | RBD | BG10NUC-LC373 |
| 6147 | RBD | BG10NUC-LC374 |
| 6148 | 2P | BG10NUC-LC375 |
| 6149 | 2P | BG10NUC-LC376 |
| 6150 | 2P | BG10NUC-LC377 |
| 6151 | 2P | BG10NUC-LC378 |
| 6152 | 2P | BG10NUC-LC379 |
| 6153 | 2P | BG10NUC-LC380 |
| 6154 | 2P | BG10NUC-LC381 |
| 6155 | RBD | BG10NUC-LC382 |
| 6156 | RBD | BG10NUC-LC383 |
| 6157 | RBD | BG10NUC-LC384 |
| 6158 | RBD | BG10NUC-LC385 |
| 6159 | RBD | BG10NUC-LC386 |
| 6160 | 2P | BG10NUC-LC387 |
| 6161 | 2P | BG10NUC-LC388 |
| 6162 | RBD | BG10NUC-LC389 |
| 6163 | 2P | BG10NUC-LC390 |
| 6164 | 2P | BG10NUC-LC391 |
| 6165 | 2P | BG10NUC-LC392 |
| 6166 | 2P | BG10NUC-LC393 |
| 6167 | 2P | BG10NUC-LC394 |
| 6168 | 2P | BG10NUC-LC395 |
| 6169 | 2P | BG10NUC-LC396 |
| 6170 | RBD | BG10NUC-LC397 |
| 6171 | 2P | BG10NUC-LC398 |
| 6172 | 2P | BG10NUC-LC399 |
| 6173 | RBD | BG10NUC-LC400 |
| 6174 | 2P | BG10NUC-LC401 |
| 6175 | 2P | BG10NUC-LC402 |
| 6176 | 2F | BG10NUC-LC403 |
| 6177 | RBD | BG10NUC-LC404 |
| 6178 | RBD | BG10NUC-LC405 |
| 6179 | 2P | BG10NUC-LC406 |
| 6180 | 2P | BG10NUC-LC407 |
| 6181 | 2P | BG10NUC-LC408 |
| 6182 | 2P | BG10NUC-LC409 |
| 6183 | RBD | BG10NUC-LC410 |
| 6184 | 2P | BG10NUC-LC411 |
| 6185 | 2P | BG10NUC-LC412 |
| 6186 | 2P | BG10NUC-LC413 |
| 6187 | RBD | BG10NUC-LC414 |
| 6188 | 2P | BG10NUC-LC415 |
| 6189 | RBD | BG10NUC-LC416 |
| 6190 | 2P | BG10NUC-LC417 |
| 6191 | RBD | BG10NUC-LC418 |
| 6192 | RBD | BG10NUC-LC419 |
| 6193 | RBD | BG10NUC-LC420 |
| 6194 | 2P | BG10NUC-LC421 |
| 6195 | RBD | BG10NUC-LC422 |
| 6196 | RBD | BG10NUC-LC423 |
| 6197 | RBD | BG10NUC-LC424 |
| 6198 | RBD | BG10NUC-LC425 |
| 6199 | 2P | BG10NUC-LC426 |
| 6200 | RBD | BG10NUC-LC427 |
| 6201 | RBD | BG13NUC-HC1 |
| 6202 | RBD | BG13NUC-HC2 |
| 6203 | RBD | BG13NUC-HC3 |
| 6204 | RBD | BG13NUC-HC4 |
| 6205 | RBD | BG13NUC-HC5 |
| 6206 | RBD | BG13NUC-HC6 |
| 6207 | RBD | BG13NUC-HC7 |
| 6208 | RBD | BG13NUC-HC8 |
| 6209 | RBD | BG13NUC-HC9 |
| 6210 | RBD | BG13NUC-HC10 |
| 6211 | RBD | BG13NUC-HC11 |
| 6212 | 2P | BG13NUC-HC12 |
| 6213 | 2P | BG13NUC-HC13 |
| 6214 | 2P | BG13NUC-HC14 |
| 6215 | 2P | BG13NUC-HC15 |
| 6216 | 2P | BG13NUC-HC16 |
| 6217 | 2P | BG13NUC-HC17 |
| 6218 | 2F | BG13NUC-HC18 |
| 6219 | 2P | BG13NUC-HC19 |
| 6220 | RBD | BG13NUC-HC20 |
| 6221 | RBD | BG13NUC-HC21 |
| 6222 | RBD | BG13NUC-HC22 |
| 6223 | RBD | BG13NUC-HC23 |
| 6224 | RBD | BG13NUC-HC24 |
| 6225 | RBD | BG13NUC-HC25 |
| 6226 | RBD | BG13NUC-HC26 |
| 6227 | 2P | BG13NUC-HC27 |
| 6228 | 2F | BG13NUC-HC28 |
| 6229 | 2F | BG13NUC-HC29 |
| 6230 | 2P | BG13NUC-HC30 |
| 6231 | 2P | BG13NUC-HC31 |
| 6232 | 2P | BG13NUC-HC32 |
| 6233 | 2P | BG13NUC-HC33 |
| 6234 | 2P | BG13NUC-HC34 |
| 6235 | RBD | BG13NUC-HC35 |
| 6236 | RBD | BG13NUC-HC36 |
| 6237 | RBD | BG13NUC-HC37 |
| 6238 | RBD | BG13NUC-HC38 |
| 6239 | RBD | BG13NUC-HC39 |
| 6240 | RBD | BG13NUC-HC40 |
| 6241 | RBD | BG13NUC-HC41 |
| 6242 | 2P | BG13NUC-HC42 |
| 6243 | 2P | BG13NUC-HC43 |
| 6244 | 2P | BG13NUC-HC44 |
| 6245 | 2P | BG13NUC-HC45 |
| 6246 | 2P | BG13NUC-HC46 |
| 6247 | RBD | BG13NUC-HC47 |
| 6248 | RBD | BG13NUC-HC48 |
| 6249 | RBD | BG13NUC-HC49 |
| 6250 | RBD | BG13NUC-HC50 |
| 6251 | RBD | BG13NUC-HC51 |
| 6252 | RBD | BG13NUC-HC52 |
| 6253 | RBD | BG13NUC-HC53 |
| 6254 | RBD | BG13NUC-HC54 |
| 6255 | RBD | BG13NUC-HC55 |
| 6256 | RBD | BG13NUC-HC56 |
| 6257 | 2P | BG13NUC-HC57 |
| 6258 | 2P | BG13NUC-HC58 |
| 6259 | RBD | BG13NUC-HC59 |
| 6260 | RBD | BG13NUC-HC60 |
| 6261 | RBD | BG13NUC-HC61 |
| 6262 | RBD | BG13NUC-HC62 |
| 6263 | RBD | BG13NUC-HC63 |
| 6264 | RBD | BG13NUC-HC64 |
| 6265 | RBD | BG13NUC-HC65 |
| 6266 | RBD | BG13NUC-HC66 |
| 6267 | 2P | BG13NUC-HC67 |
| 6268 | 2P | BG13NUC-HC68 |
| 6269 | RBD | BG13NUC-HC69 |
| 6270 | RBD | BG13NUC-HC70 |
| 6271 | RBD | BG13NUC-HC71 |
| 6272 | RBD | BG13NUC-HC72 |
| 6273 | RBD | BG13NUC-HC73 |
| 6274 | 2P | BG13NUC-HC74 |
| 6275 | 2P | BG13NUC-HC75 |
| 6276 | 2F | BG13NUC-HC76 |
| 6277 | 2P | BG13NUC-HC77 |
| 6278 | 2F | BG13NUC-HC78 |
| 6279 | RBD | BG13NUC-HC79 |
| 6280 | RBD | BG13NUC-HC80 |
| 6281 | RBD | BG13NUC-HC81 |
| 6282 | RBD | BG13NUC-HC82 |
| 6283 | RBD | BG13NUC-HC83 |
| 6284 | 2F | BG13NUC-HC84 |
| 6285 | 2P | BG13NUC-HC85 |
| 6286 | 2F | BG13NUC-HC86 |
| 6287 | RBD | BG13NUC-HC87 |
| 6288 | RBD | BG13NUC-HC88 |
| 6289 | RBD | BG13NUC-HC89 |
| 6290 | RBD | BG13NUC-HC90 |
| 6291 | RBD | BG13NUC-HC91 |
| 6292 | 2P | BG13NUC-HC92 |
| 6293 | 2P | BG13NUC-HC93 |
| 6294 | 2P | BG13NUC-HC94 |
| 6295 | RBD | BG13NUC-HC95 |
| 6296 | RBD | BG13NUC-HC96 |
| 6297 | RBD | BG13NUC-HC97 |
| 6298 | RBD | BG13NUC-HC98 |
| 6299 | RBD | BG13NUC-HC99 |
| 6300 | 2P | BG13NUC-HC100 |
| 6301 | 2P | BG13NUC-HC101 |
| 6302 | RBD | BG13NUC-HC102 |
| 6303 | RBD | BG13NUC-HC103 |
| 6304 | 2P | BG13NUC-HC104 |
| 6305 | 2P | BG13NUC-HC105 |
| 6306 | RBD | BG13NUC-HC106 |
| 6307 | RBD | BG13NUC-HC107 |
| 6308 | RBD | BG13NUC-HC108 |
| 6309 | RBD | BG13NUC-HC109 |
| 6310 | RBD | BG13NUC-HC110 |
| 6311 | 2P | BG13NUC-HC111 |
| 6312 | 2P | BG13NUC-HC112 |
| 6313 | RBD | BG13NUC-HC113 |
| 6314 | RBD | BG13NUC-HC114 |
| 6315 | RBD | BG13NUC-HC115 |
| 6316 | RBD | BG13NUC-HC116 |
| 6317 | RBD | BG13NUC-HC117 |
| 6318 | RBD | BG13NUC-HC118 |
| 6319 | RBD | BG13NUC-HC119 |
| 6320 | 2P | BG13NUC-HC120 |
| 6321 | 2P | BG13NUC-HC121 |
| 6322 | 2P | BG13NUC-HC122 |
| 6323 | RBD | BG13NUC-HC123 |
| 6324 | RBD | BG13NUC-HC124 |
| 6325 | RBD | BG13NUC-HC125 |
| 6326 | 2P | BG13NUC-HC126 |
| 6327 | 2P | BG13NUC-HC127 |
| 6328 | 2P | BG13NUC-HC128 |
| 6329 | 2P | BG13NUC-HC129 |
| 6330 | 2P | BG13NUC-HC130 |
| 6331 | 2P | BG13NUC-HC131 |
| 6332 | 2P | BG13NUC-HC132 |
| 6333 | RBD | BG13NUC-HC133 |
| 6334 | RBD | BG13NUC-HC134 |
| 6335 | 2P | BG13NUC-HC135 |
| 6336 | 2P | BG13NUC-HC136 |
| 6337 | RBD | BG13NUC-HC137 |
| 6338 | RBD | BG13NUC-HC138 |
| 6339 | RBD | BG13NUC-HC139 |
| 6340 | 2P | BG13NUC-HC140 |
| 6341 | RBD | BG13NUC-HC141 |
| 6342 | RBD | BG13NUC-HC142 |
| 6343 | RBD | BG13NUC-HC143 |
| 6344 | 2P | BG13NUC-HC144 |
| 6345 | RBD | BG13NUC-HC145 |
| 6346 | RBD | BG13NUC-HC146 |
| 6347 | RBD | BG13NUC-HC147 |
| 6348 | 2P | BG13NUC-HC148 |
| 6349 | RBD | BG13NUC-HC149 |
| 6350 | 2P | BG13NUC-HC150 |
| 6351 | 2P | BG13NUC-HC151 |
| 6352 | 2P | BG13NUC-HC152 |
| 6353 | RBD | BG13NUC-HC153 |
| 6354 | RBD | BG13NUC-HC154 |
| 6355 | 2P | BG13NUC-HC155 |
| 6356 | 2P | BG13NUC-HC156 |
| 6357 | RBD | BG13NUC-HC157 |
| 6358 | RBD | BG13NUC-HC158 |
| 6359 | RBD | BG13NUC-HC159 |
| 6360 | 2P | BG13NUC-HC160 |
| 6361 | 2P | BG13NUC-HC161 |
| 6362 | 2P | BG13NUC-HC162 |
| 6363 | RBD | BG13NUC-HC163 |
| 6364 | 2P | BG13NUC-HC164 |
| 6365 | RBD | BG13NUC-HC165 |
| 6366 | RBD | BG13NUC-HC166 |
| 6367 | RBD | BG13NUC-HC167 |
| 6368 | 2P | BG13NUC-HC168 |
| 6369 | RBD | BG13NUC-HC169 |
| 6370 | 2P | BG13NUC-HC170 |
| 6371 | RBD | BG13NUC-HC171 |
| 6372 | RBD | BG13NUC-HC172 |
| 6373 | RBD | BG13NUC-HC173 |
| 6374 | RBD | BG13NUC-HC174 |
| 6375 | RBD | BG13NUC-HC175 |
| 6376 | RBD | BG13NUC-HC176 |
| 6377 | RBD | BG13NUC-HC177 |
| 6378 | 2P | BG13NUC-HC178 |
| 6379 | 2P | BG13NUC-HC179 |
| 6380 | 2P | BG13NUC-HC180 |
| 6381 | 2P | BG13NUC-HC181 |
| 6382 | 2P | BG13NUC-HC182 |
| 6383 | 2P | BG13NUC-HC183 |
| 6384 | RBD | BG13NUC-HC184 |
| 6385 | RBD | BG13NUC-HC185 |
| 6386 | RBD | BG13NUC-HC186 |
| 6387 | RBD | BG13NUC-HC187 |
| 6388 | 2P | BG13NUC-HC188 |
| 6389 | 2P | BG13NUC-HC189 |
| 6390 | 2P | BG13NUC-HC190 |
| 6391 | RBD | BG13NUC-HC191 |
| 6392 | 2P | BG13NUC-HC192 |
| 6393 | 2P | BG13NUC-HC193 |
| 6394 | 2P | BG13NUC-HC194 |
| 6395 | RBD | BG13NUC-HC195 |
| 6396 | 2P | BG13NUC-HC196 |
| 6397 | RBD | BG13NUC-HC197 |
| 6398 | 2P | BG13NUC-HC198 |
| 6399 | RBD | BG13NUC-HC199 |
| 6400 | RBD | BG13NUC-HC200 |
| 6401 | RBD | BG13NUC-HC201 |
| 6402 | 2P | BG13NUC-HC202 |
| 6403 | 2P | BG13NUC-HC203 |
| 6404 | 2P | BG13NUC-HC204 |
| 6405 | 2P | BG13NUC-HC205 |
| 6406 | 2P | BG13NUC-HC206 |
| 6407 | RBD | BG13NUC-HC207 |
| 6408 | 2P | BG13NUC-HC208 |
| 6409 | RBD | BG13NUC-HC209 |
| 6410 | 2P | BG13NUC-HC210 |
| 6411 | RBD | BG13NUC-HC211 |
| 6412 | 2P | BG13NUC-HC212 |
| 6413 | RBD | BG13NUC-HC213 |
| 6414 | 2P | BG13NUC-HC214 |
| 6415 | RBD | BG13NUC-HC215 |
| 6416 | RBD | BG13NUC-HC216 |
| 6417 | RBD | BG13NUC-HC217 |
| 6418 | 2P | BG13NUC-HC218 |
| 6419 | RBD | BG13NUC-HC219 |
| 6420 | RBD | BG13NUC-HC220 |
| 6421 | 2P | BG13NUC-HC221 |
| 6422 | 2P | BG13NUC-HC222 |
| 6423 | RBD | BG13NUC-HC223 |
| 6424 | 2P | BG13NUC-HC224 |
| 6425 | RBD | BG13NUC-HC225 |
| 6426 | 2P | BG13NUC-HC226 |
| 6427 | 2P | BG13NUC-HC227 |
| 6428 | 2P | BG13NUC-HC228 |
| 6429 | RBD | BG13NUC-HC229 |
| 6430 | RBD | BG13NUC-HC230 |
| 6431 | RBD | BG13NUC-HC231 |
| 6432 | 2P | BG13NUC-HC232 |
| 6433 | 2P | BG13NUC-HC233 |
| 6434 | 2P | BG13NUC-HC234 |
| 6435 | RBD | BG13NUC-HC235 |
| 6436 | 2P | BG13NUC-HC236 |
| 6437 | 2P | BG13NUC-HC237 |
| 6438 | 2P | BG13NUC-HC238 |
| 6439 | RBD | BG13NUC-HC239 |
| 6440 | RBD | BG13NUC-HC240 |
| 6441 | RBD | BG13NUC-HC241 |
| 6442 | RBD | BG13NUC-HC242 |
| 6443 | RBD | BG13NUC-HC243 |
| 6444 | RBD | BG13NUC-HC244 |
| 6445 | RBD | BG13NUC-HC245 |
| 6446 | 2P | BG13NUC-HC246 |
| 6447 | RBD | BG13NUC-HC247 |
| 6448 | 2P | BG13NUC-HC248 |
| 6449 | RBD | BG13NUC-HC249 |
| 6450 | 2P | BG13NUC-HC250 |
| 6451 | 2P | BG13NUC-HC251 |
| 6452 | 2P | BG13NUC-HC252 |
| 6453 | RBD | BG13NUC-HC253 |
| 6454 | 2P | BG13NUC-HC254 |
| 6455 | RBD | BG13NUC-HC255 |
| 6456 | 2P | BG13NUC-HC256 |
| 6457 | RBD | BG13NUC-HC257 |
| 6458 | RBD | BG13NUC-HC258 |
| 6459 | 2P | BG13NUC-HC259 |
| 6460 | 2P | BG13NUC-HC260 |
| 6461 | 2P | BG13NUC-HC261 |
| 6462 | 2P | BG13NUC-HC262 |
| 6463 | RBD | BG13NUC-HC263 |
| 6464 | RBD | BG13NUC-HC264 |
| 6465 | RBD | BG13NUC-HC265 |
| 6466 | 2P | BG13NUC-HC266 |
| 6467 | RBD | BG13NUC-HC267 |
| 6468 | 2P | BG13NUC-HC268 |
| 6469 | RBD | BG13NUC-HC269 |
| 6470 | 2P | BG13NUC-HC270 |
| 6471 | 2P | BG13NUC-HC271 |
| 6472 | 2P | BG13NUC-HC272 |
| 6473 | 2P | BG13NUC-HC273 |
| 6474 | 2P | BG13NUC-HC274 |
| 6475 | 2P | BG13NUC-HC275 |
| 6476 | 2P | BG13NUC-HC276 |
| 6477 | RBD | BG13NUC-HC277 |
| 6478 | 2P | BG13NUC-HC278 |
| 6479 | 2P | BG13NUC-HC279 |
| 6480 | RBD | BG13NUC-HC280 |
| 6481 | RBD | BG13NUC-HC281 |
| 6482 | RBD | BG13NUC-HC282 |
| 6483 | RBD | BG13NUC-HC283 |
| 6484 | RBD | BG13NUC-HC284 |
| 6485 | RBD | BG13NUC-HC285 |
| 6486 | 2P | BG13NUC-HC286 |
| 6487 | 2P | BG13NUC-HC287 |
| 6488 | RBD | BG13NUC-HC288 |
| 6489 | 2P | BG13NUC-HC289 |
| 6490 | 2P | BG13NUC-HC290 |
| 6491 | 2P | BG13NUC-HC291 |
| 6492 | RBD | BG13NUC-HC292 |
| 6493 | 2P | BG13NUC-HC293 |
| 6494 | RBD | BG13NUC-HC294 |
| 6495 | RBD | BG13NUC-HC295 |
| 6496 | RBD | BG13NUC-HC296 |
| 6497 | 2P | BG13NUC-HC297 |
| 6498 | 2P | BG13NUC-HC298 |
| 6499 | RBD | BG13NUC-HC299 |
| 6500 | 2F | BG13NUC-HC300 |
| 6501 | RBD | BG13NUC-HC301 |
| 6502 | 2P | BG13NUC-HC302 |
| 6503 | RBD | BG13NUC-HC303 |
| 6504 | 2P | BG13NUC-HC304 |
| 6505 | RBD | BG13NUC-HC305 |
| 6506 | 2P | BG13NUC-HC306 |
| 6507 | 2P | BG13NUC-HC307 |
| 6508 | RBD | BG13NUC-HC308 |
| 6509 | RBD | BG13NUC-HC309 |
| 6510 | 2P | BG13NUC-HC310 |
| 6511 | RBD | BG13NUC-HC311 |
| 6512 | 2P | BG13NUC-HC312 |
| 6513 | 2P | BG13NUC-HC313 |
| 6514 | RBD | BG13NUC-HC314 |
| 6515 | 2P | BG13NUC-HC315 |
| 6516 | 2P | BG13NUC-HC316 |
| 6517 | RBD | BG13NUC-HC317 |
| 6518 | 2P | BG13NUC-HC318 |
| 6519 | RBD | BG13NUC-HC319 |
| 6520 | RBD | BG13NUC-HC320 |
| 6521 | 2P | BG13NUC-HC321 |
| 6522 | 2P | BG13NUC-HC322 |
| 6523 | 2P | BG13NUC-HC323 |
| 6524 | RBD | BG13NUC-HC324 |
| 6525 | RBD | BG13NUC-HC325 |
| 6526 | RBD | BG13NUC-HC326 |
| 6527 | 2P | BG13NUC-HC327 |
| 6528 | 2P | BG13NUC-HC328 |
| 6529 | 2P | BG13NUC-HC329 |
| 6530 | 2P | BG13NUC-HC330 |
| 6531 | RBD | BG13NUC-HC331 |
| 6532 | 2P | BG13NUC-HC332 |
| 6533 | RBD | BG13NUC-HC333 |
| 6534 | RBD | BG13NUC-HC334 |
| 6535 | 2P | BG13NUC-HC335 |
| 6536 | RBD | BG13NUC-HC336 |
| 6537 | RBD | BG13NUC-HC337 |
| 6538 | RBD | BG13NUC-HC338 |
| 6539 | RBD | BG13NUC-HC339 |
| 6540 | RBD | BG13NUC-HC340 |
| 6541 | 2P | BG13NUC-HC341 |
| 6542 | 2P | BG13NUC-HC342 |
| 6543 | 2P | BG13NUC-HC343 |
| 6544 | RBD | BG13NUC-HC344 |
| 6545 | 2P | BG13NUC-HC345 |
| 6546 | 2P | BG13NUC-HC346 |
| 6547 | RBD | BG13NUC-HC347 |
| 6548 | 2P | BG13NUC-HC348 |
| 6549 | 2P | BG13NUC-HC349 |
| 6550 | 2P | BG13NUC-HC350 |
| 6551 | 2P | BG13NUC-HC351 |
| 6552 | RBD | BG13NUC-HC352 |
| 6553 | 2P | BG13NUC-HC353 |
| 6554 | RBD | BG13NUC-HC354 |
| 6555 | 2P | BG13NUC-HC355 |
| 6556 | RBD | BG13NUC-HC356 |
| 6557 | 2P | BG13NUC-HC357 |
| 6558 | RBD | BG13NUC-HC358 |
| 6559 | 2P | BG13NUC-HC359 |
| 6560 | RBD | BG13NUC-HC360 |
| 6561 | 2P | BG13NUC-HC361 |
| 6562 | 2P | BG13NUC-HC362 |
| 6563 | RBD | BG13NUC-HC363 |
| 6564 | RBD | BG13NUC-HC364 |
| 6565 | RBD | BG13NUC-HC365 |
| 6566 | 2P | BG13NUC-HC366 |
| 6567 | 2P | BG13NUC-HC367 |
| 6568 | 2P | BG13NUC-HC368 |
| 6569 | 2P | BG13NUC-HC369 |
| 6570 | RBD | BG13NUC-HC370 |
| 6571 | 2P | BG13NUC-HC371 |
| 6572 | 2P | BG13NUC-HC372 |
| 6573 | 2P | BG13NUC-HC373 |
| 6574 | RBD | BG13NUC-HC374 |
| 6575 | 2P | BG13NUC-HC375 |
| 6576 | 2P | BG13NUC-HC376 |
| 6577 | RBD | BG13NUC-HC377 |
| 6578 | 2P | BG13NUC-HC378 |
| 6579 | RBD | BG13NUC-HC379 |
| 6580 | RBD | BG13NUC-HC380 |
| 6581 | 2P | BG13NUC-HC381 |
| 6582 | RBD | BG13NUC-HC382 |
| 6583 | RBD | BG13NUC-HC383 |
| 6584 | 2P | BG13NUC-HC384 |
| 6585 | RBD | BG13NUC-HC385 |
| 6586 | RBD | BG13NUC-HC386 |
| 6587 | RBD | BG13NUC-HC387 |
| 6588 | 2P | BG13NUC-HC388 |
| 6589 | 2P | BG13NUC-HC389 |
| 6590 | 2P | BG13NUC-HC390 |
| 6591 | 2P | BG13NUC-HC391 |
| 6592 | RBD | BG13NUC-HC392 |
| 6593 | RBD | BG13NUC-HC393 |
| 6594 | RBD | BG13NUC-HC394 |
| 6595 | 2P | BG13NUC-HC395 |
| 6596 | 2P | BG13NUC-HC396 |
| 6597 | 2P | BG13NUC-HC397 |
| 6598 | 2P | BG13NUC-HC398 |
| 6599 | 2P | BG13NUC-HC399 |
| 6600 | RBD | BG13NUC-HC400 |
| 6601 | 2P | BG13NUC-HC401 |
| 6602 | 2P | BG13NUC-HC402 |
| 6603 | 2P | BG13NUC-HC403 |
| 6604 | 2P | BG13NUC-HC404 |
| 6605 | 2P | BG13NUC-HC405 |
| 6606 | RBD | BG13NUC-HC406 |
| 6607 | 2P | BG13NUC-HC407 |
| 6608 | 2P | BG13NUC-HC408 |
| 6609 | 2P | BG13NUC-HC409 |
| 6610 | 2P | BG13NUC-HC410 |
| 6611 | RBD | BG13NUC-HC411 |
| 6612 | RBD | BG13NUC-HC412 |
| 6613 | RBD | BG13NUC-HC413 |
| 6614 | RBD | BG13NUC-HC414 |
| 6615 | 2P | BG13NUC-HC415 |
| 6616 | 2P | BG13NUC-HC416 |
| 6617 | RBD | BG13NUC-HC417 |
| 6618 | RBD | BG13NUC-HC418 |
| 6619 | 2P | BG13NUC-HC419 |
| 6620 | 2P | BG13NUC-HC420 |
| 6621 | RBD | BG13NUC-HC421 |
| 6622 | 2P | BG13NUC-HC422 |
| 6623 | 2P | BG13NUC-HC423 |
| 6624 | RBD | BG13NUC-HC424 |
| 6625 | RBD | BG13NUC-HC425 |
| 6626 | RBD | BG13NUC-HC426 |
| 6627 | 2P | BG13NUC-HC427 |
| 6628 | 2P | BG13NUC-HC428 |
| 6629 | 2P | BG13NUC-HC429 |
| 6630 | 2P | BG13NUC-HC430 |
| 6631 | 2P | BG13NUC-HC431 |
| 6632 | RBD | BG13NUC-HC432 |
| 6633 | 2P | BG13NUC-HC433 |
| 6634 | 2P | BG13NUC-HC434 |
| 6635 | 2P | BG13NUC-HC435 |
| 6636 | 2P | BG13NUC-HC436 |
| 6637 | 2P | BG13NUC-HC437 |
| 6638 | 2P | BG13NUC-HC438 |
| 6639 | RBD | BG13NUC-HC439 |
| 6640 | RBD | BG13NUC-HC440 |
| 6641 | 2P | BG13NUC-HC441 |
| 6642 | 2P | BG13NUC-HC442 |
| 6643 | 2P | BG13NUC-HC443 |
| 6644 | 2P | BG13NUC-HC444 |
| 6645 | 2P | BG13NUC-HC445 |
| 6646 | 2P | BG13NUC-HC446 |
| 6647 | 2P | BG13NUC-HC447 |
| 6648 | 2P | BG13NUC-HC448 |
| 6649 | RBD | BG13NUC-HC449 |
| 6650 | 2P | BG13NUC-HC450 |
| 6651 | 2P | BG13NUC-HC451 |
| 6652 | 2P | BG13NUC-HC452 |
| 6653 | 2P | BG13NUC-HC453 |
| 6654 | RBD | BG13NUC-HC454 |
| 6655 | RBD | BG13NUC-HC455 |
| 6656 | 2P | BG13NUC-HC456 |
| 6657 | 2P | BG13NUC-HC457 |
| 6658 | 2P | BG13NUC-HC458 |
| 6659 | RBD | BG13NUC-HC459 |
| 6660 | 2P | BG13NUC-HC460 |
| 6661 | RBD | BG13NUC-HC461 |
| 6662 | 2P | BG13NUC-HC462 |
| 6663 | RBD | BG13NUC-HC463 |
| 6664 | RBD | BG13NUC-HC464 |
| 6665 | 2P | BG13NUC-HC465 |
| 6666 | 2P | BG13NUC-HC466 |
| 6667 | 2P | BG13NUC-HC467 |
| 6668 | 2P | BG13NUC-HC468 |
| 6669 | 2P | BG13NUC-HC469 |
| 6670 | 2P | BG13NUC-HC470 |
| 6671 | RBD | BG13NUC-HC471 |
| 6672 | 2P | BG13NUC-HC472 |
| 6673 | RBD | BG13NUC-HC473 |
| 6674 | RBD | BG13NUC-HC474 |
| 6675 | 2P | BG13NUC-HC475 |
| 6676 | 2P | BG13NUC-HC476 |
| 6677 | 2P | BG13NUC-HC477 |
| 6678 | 2P | BG13NUC-HC478 |
| 6679 | 2P | BG13NUC-HC479 |
| 6680 | 2P | BG13NUC-HC480 |
| 6681 | 2P | BG13NUC-HC481 |
| 6682 | 2P | BG13NUC-HC482 |
| 6683 | 2P | BG13NUC-HC483 |
| 6684 | 2P | BG13NUC-HC484 |
| 6685 | 2P | BG13NUC-HC485 |
| 6686 | RBD | BG13NUC-HC486 |
| 6687 | RBD | BG13NUC-HC487 |
| 6688 | 2P | BG13NUC-HC488 |
| 6689 | 2P | BG13NUC-HC489 |
| 6690 | 2P | BG13NUC-HC490 |
| 6691 | 2P | BG13NUC-HC491 |
| 6692 | 2P | BG13NUC-HC492 |
| 6693 | 2P | BG13NUC-HC493 |
| 6694 | 2P | BG13NUC-HC494 |
| 6695 | 2P | BG13NUC-HC495 |
| 6696 | RBD | BG13NUC-HC496 |
| 6697 | RBD | BG13NUC-HC497 |
| 6698 | 2P | BG13NUC-HC498 |
| 6699 | RBD | BG13NUC-HC499 |
| 6700 | 2P | BG13NUC-HC500 |
| 6701 | RBD | BG13NUC-HC501 |
| 6702 | 2P | BG13NUC-HC502 |
| 6703 | 2P | BG13NUC-HC503 |
| 6704 | RBD | BG13NUC-HC504 |
| 6705 | 2P | BG13NUC-HC505 |
| 6706 | RBD | BG13NUC-HC506 |
| 6707 | RBD | BG13NUC-HC507 |
| 6708 | 2P | BG13NUC-HC508 |
| 6709 | 2P | BG13NUC-HC509 |
| 6710 | 2P | BG13NUC-HC510 |
| 6711 | 2P | BG13NUC-HC511 |
| 6712 | RBD | BG13NUC-HC512 |
| 6713 | RBD | BG13NUC-HC513 |
| 6714 | RBD | BG13NUC-HC514 |
| 6715 | 2P | BG13NUC-HC515 |
| 6716 | 2P | BG13NUC-HC516 |
| 6717 | 2P | BG13NUC-HC517 |
| 6718 | RBD | BG13NUC-HC518 |
| 6719 | RBD | BG13NUC-HC519 |
| 6720 | 2P | BG13NUC-HC520 |
| 6721 | 2P | BG13NUC-HC521 |
| 6722 | RBD | BG13NUC-HC522 |
| 6723 | RBD | BG13NUC-HC523 |
| 6724 | RBD | BG13NUC-HC524 |
| 6725 | 2P | BG13NUC-HC525 |
| 6726 | 2P | BG13NUC-HC526 |
| 6727 | 2P | BG13NUC-HC527 |
| 6728 | 2P | BG13NUC-HC528 |
| 6729 | 2P | BG13NUC-HC529 |
| 6730 | RBD | BG13NUC-HC530 |
| 6731 | 2P | BG13NUC-HC531 |
| 6732 | RBD | BG13NUC-HC532 |
| 6733 | RBD | BG13NUC-HC533 |
| 6734 | RBD | BG13NUC-HC534 |
| 6735 | RBD | BG13NUC-HC535 |
| 6736 | 2P | BG13NUC-HC536 |
| 6737 | 2P | BG13NUC-HC537 |
| 6738 | 2P | BG13NUC-HC538 |
| 6739 | 2P | BG13NUC-HC539 |
| 6740 | 2P | BG13NUC-HC540 |
| 6741 | 2P | BG13NUC-HC541 |
| 6742 | RBD | BG13NUC-HC542 |
| 6743 | RBD | BG13NUC-HC543 |
| 6744 | 2P | BG13NUC-HC544 |
| 6745 | 2P | BG13NUC-HC545 |
| 6746 | RBD | BG13NUC-HC546 |
| 6747 | RBD | BG13NUC-HC547 |
| 6748 | 2F | BG13NUC-HC548 |
| 6749 | RBD | BG13NUC-HC549 |
| 6750 | 2P | BG13NUC-HC550 |
| 6751 | 2P | BG13NUC-HC551 |
| 6752 | RBD | BG13NUC-HC552 |
| 6753 | RBD | BG13NUC-HC553 |
| 6754 | RBD | BG13NUC-HC554 |
| 6755 | RBD | BG13NUC-HC555 |
| 6756 | 2P | BG13NUC-HC556 |
| 6757 | 2P | BG13NUC-HC557 |
| 6758 | RBD | BG13NUC-HC558 |
| 6759 | 2F | BG13NUC-HC559 |
| 6760 | RBD | BG13NUC-HC560 |
| 6761 | 2P | BG13NUC-HC561 |
| 6762 | 2P | BG13NUC-HC562 |
| 6763 | 2P | BG13NUC-HC563 |
| 6764 | 2P | BG13NUC-HC564 |
| 6765 | 2F | BG13NUC-HC565 |
| 6766 | 2F | BG13NUC-HC566 |
| 6767 | 2P | BG13NUC-HC567 |
| 6768 | RBD | BG13NUC-HC568 |
| 6769 | RBD | BG13NUC-HC569 |
| 6770 | RBD | BG13NUC-HC570 |
| 6771 | RBD | BG13NUC-HC571 |
| 6772 | 2F | BG13NUC-HC572 |
| 6773 | 2P | BG13NUC-HC573 |
| 6774 | 2P | BG13NUC-HC574 |
| 6775 | 2P | BG13NUC-HC575 |
| 6776 | 2P | BG13NUC-HC576 |
| 6777 | 2P | BG13NUC-HC577 |
| 6778 | 2P | BG13NUC-HC578 |
| 6779 | 2P | BG13NUC-HC579 |
| 6780 | 2P | BG13NUC-HC580 |
| 6781 | 2F | BG13NUC-HC581 |
| 6782 | 2P | BG13NUC-HC582 |
| 6783 | 2P | BG13NUC-HC583 |
| 6784 | 2P | BG13NUC-HC584 |
| 6785 | 2P | BG13NUC-HC585 |
| 6786 | 2P | BG13NUC-HC586 |
| 6787 | 2P | BG13NUC-HC587 |
| 6788 | 2P | BG13NUC-HC588 |
| 6789 | 2P | BG13NUC-HC589 |
| 6790 | 2P | BG13NUC-HC590 |
| 6791 | RBD | BG13NUC-HC591 |
| 6792 | 2P | BG13NUC-HC592 |
| 6793 | RBD | BG13NUC-HC593 |
| 6794 | 2P | BG13NUC-HC594 |
| 6795 | RBD | BG13NUC-HC595 |
| 6796 | 2F | BG13NUC-HC596 |
| 6797 | 2F | BG13NUC-HC597 |
| 6798 | RBD | BG13NUC-HC598 |
| 6799 | 2P | BG13NUC-HC599 |
| 6800 | 2P | BG13NUC-HC600 |
| 6801 | 2P | BG13NUC-HC601 |
| 6802 | 2P | BG13NUC-HC602 |
| 6803 | 2P | BG13NUC-HC603 |
| 6804 | 2P | BG13NUC-HC604 |
| 6805 | 2P | BG13NUC-HC605 |
| 6806 | RBD | BG13NUC-HC606 |
| 6807 | RBD | BG13NUC-HC607 |
| 6808 | RBD | BG13NUC-HC608 |
| 6809 | 2P | BG13NUC-HC609 |
| 6810 | RBD | BG13NUC-HC610 |
| 6811 | 2P | BG13NUC-HC611 |
| 6812 | RBD | BG13NUC-HC612 |
| 6813 | RBD | BG13NUC-HC613 |
| 6814 | RBD | BG13NUC-HC614 |
| 6815 | 2P | BG13NUC-HC615 |
| 6816 | 2P | BG13NUC-HC616 |
| 6817 | RBD | BG13NUC-HC617 |
| 6818 | RBD | BG13NUC-HC618 |
| 6819 | 2P | BG13NUC-HC619 |
| 6820 | RBD | BG13NUC-HC620 |
| 6821 | 2P | BG13NUC-HC621 |
| 6822 | 2P | BG13NUC-HC622 |
| 6823 | RBD | BG13NUC-HC623 |
| 6824 | RBD | BG13NUC-HC624 |
| 6825 | 2P | BG13NUC-HC625 |
| 6826 | 2P | BG13NUC-HC626 |
| 6827 | 2P | BG13NUC-HC627 |
| 6828 | RBD | BG13NUC-HC628 |
| 6829 | RBD | BG13NUC-HC629 |
| 6830 | RBD | BG13NUC-HC630 |
| 6831 | 2P | BG13NUC-HC631 |
| 6832 | 2P | BG13NUC-HC632 |
| 6833 | 2P | BG13NUC-HC633 |
| 6834 | 2P | BG13NUC-HC634 |
| 6835 | 2P | BG13NUC-HC635 |
| 6836 | RBD | BG13NUC-HC636 |
| 6837 | RBD | BG13NUC-HC637 |
| 6838 | RBD | BG13NUC-HC638 |
| 6839 | 2P | BG13NUC-HC639 |
| 6840 | RBD | BG13NUC-HC640 |
| 6841 | RBD | BG13NUC-HC641 |
| 6842 | 2P | BG13NUC-HC642 |
| 6843 | 2P | BG13NUC-HC643 |
| 6844 | RBD | BG13NUC-HC644 |
| 6845 | RBD | BG13NUC-HC645 |
| 6846 | 2P | BG13NUC-HC646 |
| 6847 | RBD | BG13NUC-HC647 |
| 6848 | 2P | BG13NUC-HC648 |
| 6849 | 2P | BG13NUC-HC649 |
| 6850 | RBD | BG13NUC-HC650 |
| 6851 | RBD | BG13NUC-HC651 |
| 6852 | 2P | BG13NUC-HC652 |
| 6853 | 2P | BG13NUC-HC653 |
| 6854 | 2P | BG13NUC-HC654 |
| 6855 | 2P | BG13NUC-HC655 |
| 6856 | RBD | BG13NUC-HC656 |
| 6857 | RBD | BG13NUC-HC657 |
| 6858 | 2P | BG13NUC-HC658 |
| 6859 | 2P | BG13NUC-HC659 |
| 6860 | 2P | BG13NUC-HC660 |
| 6861 | 2P | BG13NUC-HC661 |
| 6862 | 2P | BG13NUC-HC662 |
| 6863 | 2P | BG13NUC-HC663 |
| 6864 | RBD | BG13NUC-HC664 |
| 6865 | 2P | BG13NUC-HC665 |
| 6866 | RBD | BG13NUC-HC666 |
| 6867 | 2P | BG13NUC-HC667 |
| 6868 | RBD | BG13NUC-HC668 |
| 6869 | RBD | BG13NUC-HC669 |
| 6870 | RBD | BG13NUC-HC670 |
| 6871 | 2P | BG13NUC-HC671 |
| 6872 | RBD | BG13NUC-HC672 |
| 6873 | 2P | BG13NUC-HC673 |
| 6874 | RBD | BG13NUC-HC674 |
| 6875 | RBD | BG13NUC-HC675 |
| 6876 | 2P | BG13NUC-HC676 |
| 6877 | 2P | BG13NUC-HC677 |
| 6878 | 2P | BG13NUC-HC678 |
| 6879 | RBD | BG13NUC-HC679 |
| 6880 | 2P | BG13NUC-HC680 |
| 6881 | 2P | BG13NUC-HC681 |
| 6882 | 2P | BG13NUC-HC682 |
| 6883 | 2P | BG13NUC-HC683 |
| 6884 | RBD | BG13NUC-HC684 |
| 6885 | 2P | BG13NUC-HC685 |
| 6886 | 2P | BG13NUC-HC686 |
| 6887 | 2P | BG13NUC-HC687 |
| 6888 | RBD | BG13NUC-HC688 |
| 6889 | 2P | BG13NUC-HC689 |
| 6890 | RBD | BG13NUC-HC690 |
| 6891 | 2P | BG13NUC-HC691 |
| 6892 | RBD | BG13NUC-HC692 |
| 6893 | RBD | BG13NUC-HC693 |
| 6894 | RBD | BG13NUC-HC694 |
| 6895 | 2P | BG13NUC-HC695 |
| 6896 | RBD | BG13NUC-HC696 |
| 6897 | RBD | BG13NUC-HC697 |
| 6898 | RBD | BG13NUC-HC698 |
| 6899 | 2P | BG13NUC-HC699 |
| 6900 | RBD | BG13NUC-HC700 |
| 6901 | RBD | BG13NUC-HC701 |
| 6902 | 2P | BG13NUC-HC702 |
| 6903 | 2P | BG13NUC-HC703 |
| 6904 | 2P | BG13NUC-HC704 |
| 6905 | 2P | BG13NUC-HC705 |
| 6906 | 2P | BG13NUC-HC706 |
| 6907 | 2P | BG13NUC-HC707 |
| 6908 | 2P | BG13NUC-HC708 |
| 6909 | 2P | BG13NUC-HC709 |
| 6910 | 2P | BG13NUC-HC710 |
| 6911 | 2P | BG13NUC-HC711 |
| 6912 | 2P | BG13NUC-HC712 |
| 6913 | 2P | BG13NUC-HC713 |
| 6914 | 2P | BG13NUC-HC714 |
| 6915 | RBD | BG13NUC-HC715 |
| 6916 | RBD | BG13NUC-HC716 |
| 6917 | RBD | BG13NUC-HC717 |
| 6918 | RBD | BG13NUC-HC718 |
| 6919 | RBD | BG13NUC-HC719 |
| 6920 | 2P | BG13NUC-HC720 |
| 6921 | 2P | BG13NUC-HC721 |
| 6922 | 2P | BG13NUC-HC722 |
| 6923 | 2P | BG13NUC-HC723 |
| 6924 | 2P | BG13NUC-HC724 |
| 6925 | 2P | BG13NUC-HC725 |
| 6926 | 2P | BG13NUC-HC726 |
| 6927 | 2P | BG13NUC-HC727 |
| 6928 | 2P | BG13NUC-HC728 |
| 6929 | RBD | BG13NUC-HC729 |
| 6930 | 2P | BG13NUC-HC730 |
| 6931 | 2P | BG13NUC-HC731 |
| 6932 | 2P | BG13NUC-HC732 |
| 6933 | 2P | BG13NUC-HC733 |
| 6934 | 2P | BG13NUC-HC734 |
| 6935 | 2P | BG13NUC-HC735 |
| 6936 | 2P | BG13NUC-HC736 |
| 6937 | RBD | BG13NUC-HC737 |
| 6938 | 2P | BG13NUC-HC738 |
| 6939 | RBD | BG13NUC-HC739 |
| 6940 | RBD | BG13NUC-HC740 |
| 6941 | RBD | BG13NUC-HC741 |
| 6942 | 2P | BG13NUC-HC742 |
| 6943 | 2P | BG13NUC-HC743 |
| 6944 | 2P | BG13NUC-HC744 |
| 6945 | RBD | BG13NUC-HC745 |
| 6946 | 2P | BG13NUC-HC746 |
| 6947 | RBD | BG13NUC-HC747 |
| 6948 | 2F | BG13NUC-HC748 |
| 6949 | 2P | BG13NUC-HC749 |
| 6950 | 2P | BG13NUC-HC750 |
| 6951 | 2P | BG13NUC-HC751 |
| 6952 | 2P | BG13NUC-HC752 |
| 6953 | 2P | BG13NUC-HC753 |
| 6954 | 2P | BG13NUC-HC754 |
| 6955 | 2P | BG13NUC-HC755 |
| 6956 | 2P | BG13NUC-HC756 |
| 6957 | 2P | BG13NUC-HC757 |
| 6958 | RBD | BG13NUC-HC758 |
| 6959 | 2P | BG13NUC-HC759 |
| 6960 | 2P | BG13NUC-HC760 |
| 6961 | 2P | BG13NUC-HC761 |
| 6962 | 2P | BG13NUC-HC762 |
| 6963 | RBD | BG13NUC-HC763 |
| 6964 | 2P | BG13NUC-HC764 |
| 6965 | 2P | BG13NUC-HC765 |
| 6966 | 2P | BG13NUC-HC766 |
| 6967 | 2P | BG13NUC-HC767 |
| 6968 | 2P | BG13NUC-HC768 |
| 6969 | 2P | BG13NUC-HC769 |
| 6970 | 2P | BG13NUC-HC770 |
| 6971 | 2P | BG13NUC-HC771 |
| 6972 | 2P | BG13NUC-HC772 |
| 6973 | RBD | BG13NUC-HC773 |
| 6974 | RBD | BG13NUC-HC774 |
| 6975 | RBD | BG13NUC-HC775 |
| 6976 | RBD | BG13NUC-HC776 |
| 6977 | RBD | BG13NUC-HC777 |
| 6978 | RBD | BG13NUC-HC778 |
| 6979 | 2P | BG13NUC-HC779 |
| 6980 | RBD | BG13NUC-HC780 |
| 6981 | RBD | BG13NUC-HC781 |
| 6982 | 2P | BG13NUC-HC782 |
| 6983 | 2P | BG13NUC-HC783 |
| 6984 | RBD | BG13NUC-HC784 |
| 6985 | RBD | BG13NUC-HC785 |
| 6986 | 2P | BG13NUC-HC786 |
| 6987 | 2P | BG13NUC-HC787 |
| 6988 | 2P | BG13NUC-HC788 |
| 6989 | 2P | BG13NUC-HC789 |
| 6990 | RBD | BG13NUC-HC790 |
| 6991 | 2P | BG13NUC-HC791 |
| 6992 | RBD | BG13NUC-HC792 |
| 6993 | RBD | BG13NUC-HC793 |
| 6994 | 2P | BG13NUC-HC794 |
| 6995 | 2P | BG13NUC-HC795 |
| 6996 | 2P | BG13NUC-HC796 |
| 6997 | 2P | BG13NUC-HC797 |
| 6998 | 2P | BG13NUC-HC798 |
| 6999 | RBD | BG13NUC-HC799 |
| 7000 | RBD | BG13NUC-HC800 |
| 7001 | RBD | BG13NUC-HC801 |
| 7002 | 2P | BG13NUC-HC802 |
| 7003 | 2P | BG13NUC-HC803 |
| 7004 | 2P | BG13NUC-HC804 |
| 7005 | 2P | BG13NUC-HC805 |
| 7006 | 2P | BG13NUC-HC806 |
| 7007 | 2P | BG13NUC-HC807 |
| 7008 | 2P | BG13NUC-HC808 |
| 7009 | 2P | BG13NUC-HC809 |
| 7010 | 2P | BG13NUC-HC810 |
| 7011 | RBD | BG13NUC-HC811 |
| 7012 | RBD | BG13NUC-HC812 |
| 7013 | RBD | BG13NUC-HC813 |
| 7014 | 2P | BG13NUC-HC814 |
| 7015 | 2P | BG13NUC-HC815 |
| 7016 | 2P | BG13NUC-HC816 |
| 7017 | 2P | BG13NUC-HC817 |
| 7018 | 2P | BG13NUC-HC818 |
| 7019 | 2P | BG13NUC-HC819 |
| 7020 | 2P | BG13NUC-HC820 |
| 7021 | 2P | BG13NUC-HC821 |
| 7022 | 2P | BG13NUC-HC822 |
| 7023 | RBD | BG13NUC-HC823 |
| 7024 | RBD | BG13NUC-HC824 |
| 7025 | RBD | BG13NUC-HC825 |
| 7026 | RBD | BG13NUC-HC826 |
| 7027 | RBD | BG13NUC-HC827 |
| 7028 | 2P | BG13NUC-HC828 |
| 7029 | 2P | BG13NUC-HC829 |
| 7030 | 2P | BG13NUC-HC830 |
| 7031 | 2P | BG13NUC-HC831 |
| 7032 | 2P | BG13NUC-HC832 |
| 7033 | RBD | BG13NUC-HC833 |
| 7034 | RBD | BG13NUC-HC834 |
| 7035 | RBD | BG13NUC-HC835 |
| 7036 | 2P | BG13NUC-HC836 |
| 7037 | RBD | BG13NUC-HC837 |
| 7038 | 2P | BG13NUC-HC838 |
| 7039 | RBD | BG13NUC-HC839 |
| 7040 | 2P | BG13NUC-HC840 |
| 7041 | 2P | BG13NUC-HC841 |
| 7042 | RBD | BG13NUC-HC842 |
| 7043 | RBD | BG13NUC-HC843 |
| 7044 | 2P | BG13NUC-HC844 |
| 7045 | RBD | BG13NUC-HC845 |
| 7046 | RBD | BG13NUC-HC846 |
| 7047 | 2P | BG13NUC-HC847 |
| 7048 | 2P | BG13NUC-HC848 |
| 7049 | RBD | BG13NUC-HC849 |
| 7050 | RBD | BG13NUC-HC850 |
| 7051 | RBD | BG13NUC-HC851 |
| 7052 | 2P | BG13NUC-HC852 |
| 7053 | 2P | BG13NUC-HC853 |
| 7054 | 2P | BG13NUC-HC854 |
| 7055 | 2P | BG13NUC-HC855 |
| 7056 | 2P | BG13NUC-HC856 |
| 7057 | RBD | BG13NUC-HC857 |
| 7058 | 2P | BG13NUC-HC858 |
| 7059 | 2P | BG13NUC-HC859 |
| 7060 | 2P | BG13NUC-HC860 |
| 7061 | 2P | BG13NUC-HC861 |
| 7062 | 2P | BG13NUC-HC862 |
| 7063 | RBD | BG13NUC-HC863 |
| 7064 | 2P | BG13NUC-HC864 |
| 7065 | 2P | BG13NUC-HC865 |
| 7066 | 2P | BG13NUC-HC866 |
| 7067 | 2P | BG13NUC-HC867 |
| 7068 | 2P | BG13NUC-HC868 |
| 7069 | 2P | BG13NUC-HC869 |
| 7070 | RBD | BG13NUC-HC870 |
| 7071 | 2P | BG13NUC-HC871 |
| 7072 | RBD | BG13NUC-HC872 |
| 7073 | RBD | BG13NUC-HC873 |
| 7074 | RBD | BG13NUC-HC874 |
| 7075 | RBD | BG13NUC-HC875 |
| 7076 | RBD | BG13NUC-HC876 |
| 7077 | RBD | BG13NUC-HC877 |
| 7078 | 2P | BG13NUC-HC878 |
| 7079 | 2P | BG13NUC-HC879 |
| 7080 | 2P | BG13NUC-HC880 |
| 7081 | 2P | BG13NUC-HC881 |
| 7082 | RBD | BG13NUC-HC882 |
| 7083 | 2P | BG13NUC-HC883 |
| 7084 | RBD | BG13NUC-HC884 |
| 7085 | RBD | BG13NUC-HC885 |
| 7086 | 2P | BG13NUC-HC886 |
| 7087 | RBD | BG13NUC-HC887 |
| 7088 | RBD | BG13NUC-HC888 |
| 7089 | RBD | BG13NUC-HC889 |
| 7090 | RBD | BG13NUC-HC890 |
| 7091 | RBD | BG13NUC-HC891 |
| 7092 | RBD | BG13NUC-HC892 |
| 7093 | 2P | BG13NUC-HC893 |
| 7094 | RBD | BG13NUC-HC894 |
| 7095 | RBD | BG13NUC-HC895 |
| 7096 | RBD | BG13NUC-HC896 |
| 7097 | RBD | BG13NUC-HC897 |
| 7098 | RBD | BG13NUC-HC898 |
| 7099 | RBD | BG13NUC-HC899 |
| 7100 | 2P | BG13NUC-HC900 |
| 7101 | 2P | BG13NUC-HC901 |
| 7102 | 2P | BG13NUC-HC902 |
| 7103 | 2P | BG13NUC-HC903 |
| 7104 | 2P | BG13NUC-HC904 |
| 7105 | RBD | BG13NUC-HC905 |
| 7106 | 2F | BG13NUC-HC906 |
| 7107 | RBD | BG13NUC-HC907 |
| 7108 | 2P | BG13NUC-HC908 |
| 7109 | 2P | BG13NUC-HC909 |
| 7110 | 2P | BG13NUC-HC910 |
| 7111 | 2P | BG13NUC-HC911 |
| 7112 | 2P | BG13NUC-HC912 |
| 7113 | 2P | BG13NUC-HC913 |
| 7114 | 2P | BG13NUC-HC914 |
| 7115 | 2P | BG13NUC-HC915 |
| 7116 | 2P | BG13NUC-HC916 |
| 7117 | 2P | BG13NUC-HC917 |
| 7118 | 2P | BG13NUC-HC918 |
| 7119 | RBD | BG13NUC-HC919 |
| 7120 | 2P | BG13NUC-HC920 |
| 7121 | RBD | BG13NUC-HC921 |
| 7122 | RBD | BG13NUC-HC922 |
| 7123 | RBD | BG13NUC-HC923 |
| 7124 | RBD | BG13NUC-HC924 |
| 7125 | 2F | BG13NUC-HC925 |
| 7126 | 2P | BG13NUC-HC926 |
| 7127 | RBD | BG13NUC-HC927 |
| 7128 | 2P | BG13NUC-HC928 |
| 7129 | 2P | BG13NUC-HC929 |
| 7130 | 2P | BG13NUC-HC930 |
| 7131 | 2P | BG13NUC-HC931 |
| 7132 | 2P | BG13NUC-HC932 |
| 7133 | 2P | BG13NUC-HC933 |
| 7134 | 2P | BG13NUC-HC934 |
| 7135 | RBD | BG13NUC-HC935 |
| 7136 | RBD | BG13NUC-HC936 |
| 7137 | 2P | BG13NUC-HC937 |
| 7138 | 2P | BG13NUC-HC938 |
| 7139 | 2P | BG13NUC-HC939 |
| 7140 | RBD | BG13NUC-HC940 |
| 7141 | 2P | BG13NUC-HC941 |
| 7142 | RBD | BG13NUC-HC942 |
| 7143 | RBD | BG13NUC-HC943 |
| 7144 | RBD | BG13NUC-HC944 |
| 7145 | RBD | BG13NUC-HC945 |
| 7146 | 2P | BG13NUC-HC946 |
| 7147 | RBD | BG13NUC-HC947 |
| 7148 | RBD | BG13NUC-HC948 |
| 7149 | RBD | BG13NUC-HC949 |
| 7150 | 2P | BG13NUC-HC950 |
| 7151 | RBD | BG13NUC-HC951 |
| 7152 | 2F | BG13NUC-HC952 |
| 7153 | 2P | BG13NUC-HC953 |
| 7154 | 2P | BG13NUC-HC954 |
| 7155 | 2P | BG13NUC-HC955 |
| 7156 | 2P | BG13NUC-HC956 |
| 7157 | RBD | BG13NUC-HC957 |
| 7158 | 2P | BG13NUC-HC958 |
| 7159 | RBD | BG13NUC-HC959 |
| 7160 | RBD | BG13NUC-HC960 |
| 7161 | 2P | BG13NUC-HC961 |
| 7162 | RBD | BG13NUC-HC962 |
| 7163 | RBD | BG13NUC-HC963 |
| 7164 | 2P | BG13NUC-HC964 |
| 7165 | RBD | BG13NUC-HC965 |
| 7166 | 2P | BG13NUC-HC966 |
| 7167 | 2P | BG13NUC-HC967 |
| 7168 | 2P | BG13NUC-HC968 |
| 7169 | 2P | BG13NUC-HC969 |
| 7170 | RBD | BG13NUC-HC970 |
| 7171 | 2P | BG13NUC-HC971 |
| 7172 | RBD | BG13NUC-HC972 |
| 7173 | 2P | BG13NUC-HC973 |
| 7174 | RBD | BG13NUC-HC974 |
| 7175 | RBD | BG13NUC-HC975 |
| 7176 | RBD | BG13NUC-HC976 |
| 7177 | 2P | BG13NUC-HC977 |
| 7178 | RBD | BG13NUC-HC978 |
| 7179 | 2P | BG13NUC-HC979 |
| 7180 | 2P | BG13NUC-HC980 |
| 7181 | RBD | BG13NUC-HC981 |
| 7182 | 2P | BG13NUC-HC982 |
| 7183 | 2P | BG13NUC-HC983 |
| 7184 | RBD | BG13NUC-HC984 |
| 7185 | RBD | BG13NUC-HC985 |
| 7186 | RBD | BG13NUC-HC986 |
| 7187 | RBD | BG13NUC-HC987 |
| 7188 | 2P | BG13NUC-HC988 |
| 7189 | 2P | BG13NUC-HC989 |
| 7190 | 2P | BG13NUC-HC990 |
| 7191 | RBD | BG13NUC-HC991 |
| 7192 | 2P | BG13NUC-HC992 |
| 7193 | 2P | BG13NUC-HC993 |
| 7194 | RBD | BG13NUC-HC994 |
| 7195 | RBD | BG13NUC-HC995 |
| 7196 | 2P | BG13NUC-HC996 |
| 7197 | RBD | BG13NUC-HC997 |
| 7198 | 2P | BG13NUC-HC998 |
| 7199 | RBD | BG13NUC-HC999 |
| 7200 | 2P | BG13NUC-HC1000 |
| 7201 | RBD | BG13NUC-HC1001 |
| 7202 | RBD | BG13NUC-HC1002 |
| 7203 | 2P | BG13NUC-HC1003 |
| 7204 | RBD | BG13NUC-HC1004 |
| 7205 | 2P | BG13NUC-HC1005 |
| 7206 | 2P | BG13NUC-HC1006 |
| 7207 | 2P | BG13NUC-HC1007 |
| 7208 | 2P | BG13NUC-HC1008 |
| 7209 | 2P | BG13NUC-HC1009 |
| 7210 | RBD | BG13NUC-HC1010 |
| 7211 | 2P | BG13NUC-HC1011 |
| 7212 | 2P | BG13NUC-HC1012 |
| 7213 | RBD | BG13NUC-HC1013 |
| 7214 | RBD | BG13NUC-HC1014 |
| 7215 | 2P | BG13NUC-HC1015 |
| 7216 | 2P | BG13NUC-HC1016 |
| 7217 | 2P | BG13NUC-HC1017 |
| 7218 | 2P | BG13NUC-HC1018 |
| 7219 | 2P | BG13NUC-HC1019 |
| 7220 | 2P | BG13NUC-HC1020 |
| 7221 | 2P | BG13NUC-HC1021 |
| 7222 | RBD | BG13NUC-HC1022 |
| 7223 | 2P | BG13NUC-HC1023 |
| 7224 | 2P | BG13NUC-HC1024 |
| 7225 | 2P | BG13NUC-HC1025 |
| 7226 | RBD | BG13NUC-HC1026 |
| 7227 | 2P | BG13NUC-HC1027 |
| 7228 | 2P | BG13NUC-HC1028 |
| 7229 | 2P | BG13NUC-HC1029 |
| 7230 | 2P | BG13NUC-HC1030 |
| 7231 | 2P | BG13NUC-HC1031 |
| 7232 | RBD | BG13NUC-HC1032 |
| 7233 | RBD | BG13NUC-HC1033 |
| 7234 | 2P | BG13NUC-HC1034 |
| 7235 | 2P | BG13NUC-HC1035 |
| 7236 | 2P | BG13NUC-HC1036 |
| 7237 | 2P | BG13NUC-HC1037 |
| 7238 | RBD | BG13NUC-HC1038 |
| 7239 | RBD | BG13NUC-HC1039 |
| 7240 | RBD | BG13NUC-HC1040 |
| 7241 | 2P | BG13NUC-HC1041 |
| 7242 | 2F | BG13NUC-HC1042 |
| 7243 | RBD | BG13NUC-HC1043 |
| 7244 | 2P | BG13NUC-HC1044 |
| 7245 | 2P | BG13NUC-HC1045 |
| 7246 | 2P | BG13NUC-HC1046 |
| 7247 | 2P | BG13NUC-HC1047 |
| 7248 | 2P | BG13NUC-HC1048 |
| 7249 | RBD | BG13NUC-HC1049 |
| 7250 | 2P | BG13NUC-HC1050 |
| 7251 | 2P | BG13NUC-HC1051 |
| 7252 | 2P | BG13NUC-HC1052 |
| 7253 | RBD | BG13NUC-HC1053 |
| 7254 | 2P | BG13NUC-HC1054 |
| 7255 | 2P | BG13NUC-HC1055 |
| 7256 | 2P | BG13NUC-HC1056 |
| 7257 | 2F | BG13NUC-HC1057 |
| 7258 | 2P | BG13NUC-HC1058 |
| 7259 | RBD | BG13NUC-HC1059 |
| 7260 | 2P | BG13NUC-HC1060 |
| 7261 | RBD | BG13NUC-HC1061 |
| 7262 | 2P | BG13NUC-HC1062 |
| 7263 | 2P | BG13NUC-HC1063 |
| 7264 | 2P | BG13NUC-HC1064 |
| 7265 | RBD | BG13NUC-HC1065 |
| 7266 | 2P | BG13NUC-HC1066 |
| 7267 | 2P | BG13NUC-HC1067 |
| 7268 | RBD | BG13NUC-HC1068 |
| 7269 | 2P | BG13NUC-HC1069 |
| 7270 | 2P | BG13NUC-HC1070 |
| 7271 | 2P | BG13NUC-HC1071 |
| 7272 | RBD | BG13NUC-HC1072 |
| 7273 | RBD | BG13NUC-HC1073 |
| 7274 | 2P | BG13NUC-HC1074 |
| 7275 | RBD | BG13NUC-HC1075 |
| 7276 | 2P | BG13NUC-HC1076 |
| 7277 | 2P | BG13NUC-HC1077 |
| 7278 | RBD | BG13NUC-HC1078 |
| 7279 | RBD | BG13NUC-HC1079 |
| 7280 | RBD | BG13NUC-HC1080 |
| 7281 | RBD | BG13NUC-HC1081 |
| 7282 | 2P | BG13NUC-HC1082 |
| 7283 | 2P | BG13NUC-HC1083 |
| 7284 | RBD | BG13NUC-HC1084 |
| 7285 | 2P | BG13NUC-HC1085 |
| 7286 | RBD | BG13NUC-HC1086 |
| 7287 | 2P | BG13NUC-HC1087 |
| 7288 | 2P | BG13NUC-HC1088 |
| 7289 | 2P | BG13NUC-HC1089 |
| 7290 | RBD | BG13NUC-HC1090 |
| 7291 | RBD | BG13NUC-HC1091 |
| 7292 | 2P | BG13NUC-HC1092 |
| 7293 | RBD | BG13NUC-HC1093 |
| 7294 | 2P | BG13NUC-HC1094 |
| 7295 | RBD | BG13NUC-HC1095 |
| 7296 | RBD | BG13NUC-HC1096 |
| 7297 | RBD | BG13NUC-HC1097 |
| 7298 | RBD | BG13NUC-HC1098 |
| 7299 | 2P | BG13NUC-HC1099 |
| 7300 | RBD | BG13NUC-HC1100 |
| 7301 | RBD | BG13NUC-HC1101 |
| 7302 | RBD | BG13NUC-HC1102 |
| 7303 | RBD | BG13NUC-HC1103 |
| 7304 | 2P | BG13NUC-HC1104 |
| 7305 | 2P | BG13NUC-HC1105 |
| 7306 | RBD | BG13NUC-HC1106 |
| 7307 | 2P | BG13NUC-HC1107 |
| 7308 | RBD | BG13NUC-HC1108 |
| 7309 | 2P | BG13NUC-HC1109 |
| 7310 | 2F | BG13NUC-HC1110 |
| 7311 | 2P | BG13NUC-HC1111 |
| 7312 | RBD | BG13NUC-HC1112 |
| 7313 | 2P | BG13NUC-HC1113 |
| 7314 | RBD | BG13NUC-HC1114 |
| 7315 | 2P | BG13NUC-HC1115 |
| 7316 | 2P | BG13NUC-HC1116 |
| 7317 | 2P | BG13NUC-HC1117 |
| 7318 | 2P | BG13NUC-HC1118 |
| 7319 | 2P | BG13NUC-HC1119 |
| 7320 | 2P | BG13NUC-HC1120 |
| 7321 | 2P | BG13NUC-HC1121 |
| 7322 | 2P | BG13NUC-HC1122 |
| 7323 | 2P | BG13NUC-HC1123 |
| 7324 | 2P | BG13NUC-HC1124 |
| 7325 | 2P | BG13NUC-HC1125 |
| 7326 | RBD | BG13NUC-HC1126 |
| 7327 | 2P | BG13NUC-HC1127 |
| 7328 | 2P | BG13NUC-HC1128 |
| 7329 | 2P | BG13NUC-HC1129 |
| 7330 | 2P | BG13NUC-HC1130 |
| 7331 | RBD | BG13NUC-HC1131 |
| 7332 | RBD | BG13NUC-HC1132 |
| 7333 | 2P | BG13NUC-HC1133 |
| 7334 | RBD | BG13NUC-HC1134 |
| 7335 | RBD | BG13NUC-HC1135 |
| 7336 | RBD | BG13NUC-HC1136 |
| 7337 | RBD | BG13NUC-LC1 |
| 7338 | RBD | BG13NUC-LC2 |
| 7339 | RBD | BG13NUC-LC3 |
| 7340 | RBD | BG13NUC-LC4 |
| 7341 | RBD | BG13NUC-LC5 |
| 7342 | RBD | BG13NUC-LC6 |
| 7343 | RBD | BG13NUC-LC7 |
| 7344 | RBD | BG13NUC-LC8 |
| 7345 | RBD | BG13NUC-LC9 |
| 7346 | RBD | BG13NUC-LC10 |
| 7347 | RBD | BG13NUC-LC11 |
| 7348 | 2P | BG13NUC-LC12 |
| 7349 | 2P | BG13NUC-LC13 |
| 7350 | 2P | BG13NUC-LC14 |
| 7351 | 2P | BG13NUC-LC15 |
| 7352 | 2P | BG13NUC-LC16 |
| 7353 | 2P | BG13NUC-LC17 |
| 7354 | 2F | BG13NUC-LC18 |
| 7355 | 2P | BG13NUC-LC19 |
| 7356 | RBD | BG13NUC-LC20 |
| 7357 | RBD | BG13NUC-LC21 |
| 7358 | RBD | BG13NUC-LC22 |
| 7359 | RBD | BG13NUC-LC23 |
| 7360 | RBD | BG13NUC-LC24 |
| 7361 | RBD | BG13NUC-LC25 |
| 7362 | RBD | BG13NUC-LC26 |
| 7363 | 2P | BG13NUC-LC27 |
| 7364 | 2P | BG13NUC-LC28 |
| 7365 | 2P | BG13NUC-LC29 |
| 7366 | 2P | BG13NUC-LC30 |
| 7367 | 2P | BG13NUC-LC31 |
| 7368 | 2P | BG13NUC-LC32 |
| 7369 | 2P | BG13NUC-LC33 |
| 7370 | 2P | BG13NUC-LC34 |
| 7371 | RBD | BG13NUC-LC35 |
| 7372 | RBD | BG13NUC-LC36 |
| 7373 | RBD | BG13NUC-LC37 |
| 7374 | RBD | BG13NUC-LC38 |
| 7375 | RBD | BG13NUC-LC39 |
| 7376 | RBD | BG13NUC-LC40 |
| 7377 | RBD | BG13NUC-LC41 |
| 7378 | 2P | BG13NUC-LC42 |
| 7379 | 2P | BG13NUC-LC43 |
| 7380 | 2P | BG13NUC-LC44 |
| 7381 | 2P | BG13NUC-LC45 |
| 7382 | 2P | BG13NUC-LC46 |
| 7383 | RBD | BG13NUC-LC47 |
| 7384 | RBD | BG13NUC-LC48 |
| 7385 | RBD | BG13NUC-LC49 |
| 7386 | RBD | BG13NUC-LC50 |
| 7387 | RBD | BG13NUC-LC51 |
| 7388 | RBD | BG13NUC-LC52 |
| 7389 | RBD | BG13NUC-LC53 |
| 7390 | RBD | BG13NUC-LC54 |
| 7391 | RBD | BG13NUC-LC55 |
| 7392 | RBD | BG13NUC-LC56 |
| 7393 | 2P | BG13NUC-LC57 |
| 7394 | 2P | BG13NUC-LC58 |
| 7395 | RBD | BG13NUC-LC59 |
| 7396 | RBD | BG13NUC-LC60 |
| 7397 | RBD | BG13NUC-LC61 |
| 7398 | RBD | BG13NUC-LC62 |
| 7399 | RBD | BG13NUC-LC63 |
| 7400 | RBD | BG13NUC-LC64 |
| 7401 | RBD | BG13NUC-LC65 |
| 7402 | RBD | BG13NUC-LC66 |
| 7403 | 2P | BG13NUC-LC67 |
| 7404 | 2P | BG13NUC-LC68 |
| 7405 | RBD | BG13NUC-LC69 |
| 7406 | RBD | BG13NUC-LC70 |
| 7407 | RBD | BG13NUC-LC71 |
| 7408 | RBD | BG13NUC-LC72 |
| 7409 | RBD | BG13NUC-LC73 |
| 7410 | 2P | BG13NUC-LC74 |
| 7411 | 2P | BG13NUC-LC75 |
| 7412 | 2P | BG13NUC-LC76 |
| 7413 | 2P | BG13NUC-LC77 |
| 7414 | 2P | BG13NUC-LC78 |
| 7415 | RBD | BG13NUC-LC79 |
| 7416 | RBD | BG13NUC-LC80 |
| 7417 | RBD | BG13NUC-LC81 |
| 7418 | RBD | BG13NUC-LC82 |
| 7419 | RBD | BG13NUC-LC83 |
| 7420 | 2P | BG13NUC-LC84 |
| 7421 | 2P | BG13NUC-LC85 |
| 7422 | 2P | BG13NUC-LC86 |
| 7423 | RBD | BG13NUC-LC87 |
| 7424 | RBD | BG13NUC-LC88 |
| 7425 | RBD | BG13NUC-LC89 |
| 7426 | RBD | BG13NUC-LC90 |
| 7427 | RBD | BG13NUC-LC91 |
| 7428 | 2P | BG13NUC-LC92 |
| 7429 | 2P | BG13NUC-LC93 |
| 7430 | 2P | BG13NUC-LC94 |
| 7431 | RBD | BG13NUC-LC95 |
| 7432 | RBD | BG13NUC-LC96 |
| 7433 | RBD | BG13NUC-LC97 |
| 7434 | RBD | BG13NUC-LC98 |
| 7435 | RBD | BG13NUC-LC99 |
| 7436 | 2P | BG13NUC-LC100 |
| 7437 | 2P | BG13NUC-LC101 |
| 7438 | RBD | BG13NUC-LC102 |
| 7439 | RBD | BG13NUC-LC103 |
| 7440 | 2P | BG13NUC-LC104 |
| 7441 | 2P | BG13NUC-LC105 |
| 7442 | RBD | BG13NUC-LC106 |
| 7443 | RBD | BG13NUC-LC107 |
| 7444 | RBD | BG13NUC-LC108 |
| 7445 | RBD | BG13NUC-LC109 |
| 7446 | RBD | BG13NUC-LC110 |
| 7447 | 2P | BG13NUC-LC111 |
| 7448 | 2P | BG13NUC-LC112 |
| 7449 | RBD | BG13NUC-LC113 |
| 7450 | RBD | BG13NUC-LC114 |
| 7451 | RBD | BG13NUC-LC115 |
| 7452 | RBD | BG13NUC-LC116 |
| 7453 | RBD | BG13NUC-LC117 |
| 7454 | RBD | BG13NUC-LC118 |
| 7455 | RBD | BG13NUC-LC119 |
| 7456 | 2P | BG13NUC-LC120 |
| 7457 | 2P | BG13NUC-LC121 |
| 7458 | 2P | BG13NUC-LC122 |
| 7459 | RBD | BG13NUC-LC123 |
| 7460 | RBD | BG13NUC-LC124 |
| 7461 | RBD | BG13NUC-LC125 |
| 7462 | 2P | BG13NUC-LC126 |
| 7463 | 2P | BG13NUC-LC127 |
| 7464 | 2P | BG13NUC-LC128 |
| 7465 | 2P | BG13NUC-LC129 |
| 7466 | 2P | BG13NUC-LC130 |
| 7467 | 2P | BG13NUC-LC131 |
| 7468 | 2P | BG13NUC-LC132 |
| 7469 | RBD | BG13NUC-LC133 |
| 7470 | RBD | BG13NUC-LC134 |
| 7471 | 2P | BG13NUC-LC135 |
| 7472 | 2P | BG13NUC-LC136 |
| 7473 | RBD | BG13NUC-LC137 |
| 7474 | RBD | BG13NUC-LC138 |
| 7475 | RBD | BG13NUC-LC139 |
| 7476 | 2P | BG13NUC-LC140 |
| 7477 | RBD | BG13NUC-LC141 |
| 7478 | RBD | BG13NUC-LC142 |
| 7479 | RBD | BG13NUC-LC143 |
| 7480 | 2P | BG13NUC-LC144 |
| 7481 | RBD | BG13NUC-LC145 |
| 7482 | RBD | BG13NUC-LC146 |
| 7483 | RBD | BG13NUC-LC147 |
| 7484 | 2P | BG13NUC-LC148 |
| 7485 | RBD | BG13NUC-LC149 |
| 7486 | 2P | BG13NUC-LC150 |
| 7487 | 2P | BG13NUC-LC151 |
| 7488 | 2P | BG13NUC-LC152 |
| 7489 | RBD | BG13NUC-LC153 |
| 7490 | RBD | BG13NUC-LC154 |
| 7491 | 2P | BG13NUC-LC155 |
| 7492 | 2P | BG13NUC-LC156 |
| 7493 | RBD | BG13NUC-LC157 |
| 7494 | RBD | BG13NUC-LC158 |
| 7495 | RBD | BG13NUC-LC159 |
| 7496 | 2P | BG13NUC-LC160 |
| 7497 | 2P | BG13NUC-LC161 |
| 7498 | 2P | BG13NUC-LC162 |
| 7499 | RBD | BG13NUC-LC163 |
| 7500 | 2P | BG13NUC-LC164 |
| 7501 | RBD | BG13NUC-LC165 |
| 7502 | RBD | BG13NUC-LC166 |
| 7503 | RBD | BG13NUC-LC167 |
| 7504 | 2P | BG13NUC-LC168 |
| 7505 | RBD | BG13NUC-LC169 |
| 7506 | 2P | BG13NUC-LC170 |
| 7507 | RBD | BG13NUC-LC171 |
| 7508 | RBD | BG13NUC-LC172 |
| 7509 | RBD | BG13NUC-LC173 |
| 7510 | RBD | BG13NUC-LC174 |
| 7511 | RBD | BG13NUC-LC175 |
| 7512 | RBD | BG13NUC-LC176 |
| 7513 | RBD | BG13NUC-LC177 |
| 7514 | 2P | BG13NUC-LC178 |
| 7515 | 2P | BG13NUC-LC179 |
| 7516 | 2P | BG13NUC-LC180 |
| 7517 | 2P | BG13NUC-LC181 |
| 7518 | 2P | BG13NUC-LC182 |
| 7519 | 2P | BG13NUC-LC183 |
| 7520 | RBD | BG13NUC-LC184 |
| 7521 | RBD | BG13NUC-LC185 |
| 7522 | RBD | BG13NUC-LC186 |
| 7523 | RBD | BG13NUC-LC187 |
| 7524 | 2P | BG13NUC-LC188 |
| 7525 | 2P | BG13NUC-LC189 |
| 7526 | 2P | BG13NUC-LC190 |
| 7527 | RBD | BG13NUC-LC191 |
| 7528 | 2P | BG13NUC-LC192 |
| 7529 | 2P | BG13NUC-LC193 |
| 7530 | 2P | BG13NUC-LC194 |
| 7531 | RBD | BG13NUC-LC195 |
| 7532 | 2P | BG13NUC-LC196 |
| 7533 | RBD | BG13NUC-LC197 |
| 7534 | 2P | BG13NUC-LC198 |
| 7535 | RBD | BG13NUC-LC199 |
| 7536 | RBD | BG13NUC-LC200 |
| 7537 | RBD | BG13NUC-LC201 |
| 7538 | 2P | BG13NUC-LC202 |
| 7539 | 2P | BG13NUC-LC203 |
| 7540 | 2P | BG13NUC-LC204 |
| 7541 | 2P | BG13NUC-LC205 |
| 7542 | 2P | BG13NUC-LC206 |
| 7543 | RBD | BG13NUC-LC207 |
| 7544 | 2P | BG13NUC-LC208 |
| 7545 | RBD | BG13NUC-LC209 |
| 7546 | 2P | BG13NUC-LC210 |
| 7547 | RBD | BG13NUC-LC211 |
| 7548 | 2P | BG13NUC-LC212 |
| 7549 | RBD | BG13NUC-LC213 |
| 7550 | 2P | BG13NUC-LC214 |
| 7551 | RBD | BG13NUC-LC215 |
| 7552 | RBD | BG13NUC-LC216 |
| 7553 | RBD | BG13NUC-LC217 |
| 7554 | 2P | BG13NUC-LC218 |
| 7555 | RBD | BG13NUC-LC219 |
| 7556 | RBD | BG13NUC-LC220 |
| 7557 | 2P | BG13NUC-LC221 |
| 7558 | 2P | BG13NUC-LC222 |
| 7559 | RBD | BG13NUC-LC223 |
| 7560 | 2P | BG13NUC-LC224 |
| 7561 | RBD | BG13NUC-LC225 |
| 7562 | 2P | BG13NUC-LC226 |
| 7563 | 2P | BG13NUC-LC227 |
| 7564 | 2P | BG13NUC-LC228 |
| 7565 | RBD | BG13NUC-LC229 |
| 7566 | RBD | BG13NUC-LC230 |
| 7567 | RBD | BG13NUC-LC231 |
| 7568 | 2P | BG13NUC-LC232 |
| 7569 | 2P | BG13NUC-LC233 |
| 7570 | 2P | BG13NUC-LC234 |
| 7571 | RBD | BG13NUC-LC235 |
| 7572 | 2P | BG13NUC-LC236 |
| 7573 | 2P | BG13NUC-LC237 |
| 7574 | 2P | BG13NUC-LC238 |
| 7575 | RBD | BG13NUC-LC239 |
| 7576 | RBD | BG13NUC-LC240 |
| 7577 | RBD | BG13NUC-LC241 |
| 7578 | RBD | BG13NUC-LC242 |
| 7579 | RBD | BG13NUC-LC243 |
| 7580 | RBD | BG13NUC-LC244 |
| 7581 | RBD | BG13NUC-LC245 |
| 7582 | 2P | BG13NUC-LC246 |
| 7583 | RBD | BG13NUC-LC247 |
| 7584 | 2P | BG13NUC-LC248 |
| 7585 | RBD | BG13NUC-LC249 |
| 7586 | 2P | BG13NUC-LC250 |
| 7587 | 2P | BG13NUC-LC251 |
| 7588 | 2P | BG13NUC-LC252 |
| 7589 | RBD | BG13NUC-LC253 |
| 7590 | 2P | BG13NUC-LC254 |
| 7591 | RBD | BG13NUC-LC255 |
| 7592 | 2P | BG13NUC-LC256 |
| 7593 | RBD | BG13NUC-LC257 |
| 7594 | RBD | BG13NUC-LC258 |
| 7595 | 2P | BG13NUC-LC259 |
| 7596 | 2P | BG13NUC-LC260 |
| 7597 | 2P | BG13NUC-LC261 |
| 7598 | 2P | BG13NUC-LC262 |
| 7599 | RBD | BG13NUC-LC263 |
| 7600 | RBD | BG13NUC-LC264 |
| 7601 | RBD | BG13NUC-LC265 |
| 7602 | 2P | BG13NUC-LC266 |
| 7603 | RBD | BG13NUC-LC267 |
| 7604 | 2P | BG13NUC-LC268 |
| 7605 | RBD | BG13NUC-LC269 |
| 7606 | 2P | BG13NUC-LC270 |
| 7607 | 2P | BG13NUC-LC271 |
| 7608 | 2P | BG13NUC-LC272 |
| 7609 | 2F | BG13NUC-LC273 |
| 7610 | 2P | BG13NUC-LC274 |
| 7611 | 2P | BG13NUC-LC275 |
| 7612 | 2P | BG13NUC-LC276 |
| 7613 | RBD | BG13NUC-LC277 |
| 7614 | 2P | BG13NUC-LC278 |
| 7615 | 2P | BG13NUC-LC279 |
| 7616 | RBD | BG13NUC-LC280 |
| 7617 | RBD | BG13NUC-LC281 |
| 7618 | RBD | BG13NUC-LC282 |
| 7619 | RBD | BG13NUC-LC283 |
| 7620 | RBD | BG13NUC-LC284 |
| 7621 | RBD | BG13NUC-LC285 |
| 7622 | 2P | BG13NUC-LC286 |
| 7623 | 2P | BG13NUC-LC287 |
| 7624 | RBD | BG13NUC-LC288 |
| 7625 | 2P | BG13NUC-LC289 |
| 7626 | 2P | BG13NUC-LC290 |
| 7627 | 2P | BG13NUC-LC291 |
| 7628 | RBD | BG13NUC-LC292 |
| 7629 | 2P | BG13NUC-LC293 |
| 7630 | RBD | BG13NUC-LC294 |
| 7631 | RBD | BG13NUC-LC295 |
| 7632 | RBD | BG13NUC-LC296 |
| 7633 | 2F | BG13NUC-LC297 |
| 7634 | 2P | BG13NUC-LC298 |
| 7635 | RBD | BG13NUC-LC299 |
| 7636 | 2P | BG13NUC-LC300 |
| 7637 | RBD | BG13NUC-LC301 |
| 7638 | 2P | BG13NUC-LC302 |
| 7639 | RBD | BG13NUC-LC303 |
| 7640 | 2P | BG13NUC-LC304 |
| 7641 | RBD | BG13NUC-LC305 |
| 7642 | 2P | BG13NUC-LC306 |
| 7643 | 2P | BG13NUC-LC307 |
| 7644 | RBD | BG13NUC-LC308 |
| 7645 | RBD | BG13NUC-LC309 |
| 7646 | 2P | BG13NUC-LC310 |
| 7647 | RBD | BG13NUC-LC311 |
| 7648 | 2P | BG13NUC-LC312 |
| 7649 | 2P | BG13NUC-LC313 |
| 7650 | RBD | BG13NUC-LC314 |
| 7651 | 2P | BG13NUC-LC315 |
| 7652 | 2P | BG13NUC-LC316 |
| 7653 | RBD | BG13NUC-LC317 |
| 7654 | 2P | BG13NUC-LC318 |
| 7655 | RBD | BG13NUC-LC319 |
| 7656 | RBD | BG13NUC-LC320 |
| 7657 | 2P | BG13NUC-LC321 |
| 7658 | 2P | BG13NUC-LC322 |
| 7659 | 2P | BG13NUC-LC323 |
| 7660 | RBD | BG13NUC-LC324 |
| 7661 | RBD | BG13NUC-LC325 |
| 7662 | RBD | BG13NUC-LC326 |
| 7663 | 2P | BG13NUC-LC327 |
| 7664 | 2P | BG13NUC-LC328 |
| 7665 | 2P | BG13NUC-LC329 |
| 7666 | 2P | BG13NUC-LC330 |
| 7667 | RBD | BG13NUC-LC331 |
| 7668 | 2P | BG13NUC-LC332 |
| 7669 | RBD | BG13NUC-LC333 |
| 7670 | RBD | BG13NUC-LC334 |
| 7671 | 2P | BG13NUC-LC335 |
| 7672 | RBD | BG13NUC-LC336 |
| 7673 | RBD | BG13NUC-LC337 |
| 7674 | RBD | BG13NUC-LC338 |
| 7675 | RBD | BG13NUC-LC339 |
| 7676 | RBD | BG13NUC-LC340 |
| 7677 | 2P | BG13NUC-LC341 |
| 7678 | 2P | BG13NUC-LC342 |
| 7679 | 2P | BG13NUC-LC343 |
| 7680 | RBD | BG13NUC-LC344 |
| 7681 | 2P | BG13NUC-LC345 |
| 7682 | 2P | BG13NUC-LC346 |
| 7683 | RBD | BG13NUC-LC347 |
| 7684 | 2P | BG13NUC-LC348 |
| 7685 | 2P | BG13NUC-LC349 |
| 7686 | 2P | BG13NUC-LC350 |
| 7687 | 2P | BG13NUC-LC351 |
| 7688 | RBD | BG13NUC-LC352 |
| 7689 | 2P | BG13NUC-LC353 |
| 7690 | RBD | BG13NUC-LC354 |
| 7691 | 2P | BG13NUC-LC355 |
| 7692 | RBD | BG13NUC-LC356 |
| 7693 | 2P | BG13NUC-LC357 |
| 7694 | RBD | BG13NUC-LC358 |
| 7695 | 2P | BG13NUC-LC359 |
| 7696 | RBD | BG13NUC-LC360 |
| 7697 | 2P | BG13NUC-LC361 |
| 7698 | 2P | BG13NUC-LC362 |
| 7699 | RBD | BG13NUC-LC363 |
| 7700 | RBD | BG13NUC-LC364 |
| 7701 | RBD | BG13NUC-LC365 |
| 7702 | 2P | BG13NUC-LC366 |
| 7703 | 2P | BG13NUC-LC367 |
| 7704 | 2P | BG13NUC-LC368 |
| 7705 | 2P | BG13NUC-LC369 |
| 7706 | RBD | BG13NUC-LC370 |
| 7707 | 2P | BG13NUC-LC371 |
| 7708 | 2P | BG13NUC-LC372 |
| 7709 | 2P | BG13NUC-LC373 |
| 7710 | RBD | BG13NUC-LC374 |
| 7711 | 2P | BG13NUC-LC375 |
| 7712 | 2P | BG13NUC-LC376 |
| 7713 | RBD | BG13NUC-LC377 |
| 7714 | 2P | BG13NUC-LC378 |
| 7715 | RBD | BG13NUC-LC379 |
| 7716 | RBD | BG13NUC-LC380 |
| 7717 | 2P | BG13NUC-LC381 |
| 7718 | RBD | BG13NUC-LC382 |
| 7719 | RBD | BG13NUC-LC383 |
| 7720 | 2P | BG13NUC-LC384 |
| 7721 | RBD | BG13NUC-LC385 |
| 7722 | RBD | BG13NUC-LC386 |
| 7723 | RBD | BG13NUC-LC387 |
| 7724 | 2P | BG13NUC-LC388 |
| 7725 | 2P | BG13NUC-LC389 |
| 7726 | 2P | BG13NUC-LC390 |
| 7727 | 2P | BG13NUC-LC391 |
| 7728 | RBD | BG13NUC-LC392 |
| 7729 | RBD | BG13NUC-LC393 |
| 7730 | RBD | BG13NUC-LC394 |
| 7731 | 2P | BG13NUC-LC395 |
| 7732 | 2P | BG13NUC-LC396 |
| 7733 | 2P | BG13NUC-LC397 |
| 7734 | 2P | BG13NUC-LC398 |
| 7735 | 2P | BG13NUC-LC399 |
| 7736 | RBD | BG13NUC-LC400 |
| 7737 | 2P | BG13NUC-LC401 |
| 7738 | 2P | BG13NUC-LC402 |
| 7739 | 2P | BG13NUC-LC403 |
| 7740 | 2P | BG13NUC-LC404 |
| 7741 | 2P | BG13NUC-LC405 |
| 7742 | RBD | BG13NUC-LC406 |
| 7743 | 2P | BG13NUC-LC407 |
| 7744 | 2P | BG13NUC-LC408 |
| 7745 | 2P | BG13NUC-LC409 |
| 7746 | 2P | BG13NUC-LC410 |
| 7747 | RBD | BG13NUC-LC411 |
| 7748 | RBD | BG13NUC-LC412 |
| 7749 | RBD | BG13NUC-LC413 |
| 7750 | RBD | BG13NUC-LC414 |
| 7751 | 2P | BG13NUC-LC415 |
| 7752 | 2P | BG13NUC-LC416 |
| 7753 | RBD | BG13NUC-LC417 |
| 7754 | RBD | BG13NUC-LC418 |
| 7755 | 2P | BG13NUC-LC419 |
| 7756 | 2P | BG13NUC-LC420 |
| 7757 | RBD | BG13NUC-LC421 |
| 7758 | 2P | BG13NUC-LC422 |
| 7759 | 2P | BG13NUC-LC423 |
| 7760 | RBD | BG13NUC-LC424 |
| 7761 | RBD | BG13NUC-LC425 |
| 7762 | RBD | BG13NUC-LC426 |
| 7763 | 2P | BG13NUC-LC427 |
| 7764 | 2P | BG13NUC-LC428 |
| 7765 | 2P | BG13NUC-LC429 |
| 7766 | 2P | BG13NUC-LC430 |
| 7767 | 2P | BG13NUC-LC431 |
| 7768 | RBD | BG13NUC-LC432 |
| 7769 | 2P | BG13NUC-LC433 |
| 7770 | 2P | BG13NUC-LC434 |
| 7771 | 2P | BG13NUC-LC435 |
| 7772 | 2P | BG13NUC-LC436 |
| 7773 | 2P | BG13NUC-LC437 |
| 7774 | 2P | BG13NUC-LC438 |
| 7775 | RBD | BG13NUC-LC439 |
| 7776 | RBD | BG13NUC-LC440 |
| 7777 | 2P | BG13NUC-LC441 |
| 7778 | 2P | BG13NUC-LC442 |
| 7779 | 2P | BG13NUC-LC443 |
| 7780 | 2P | BG13NUC-LC444 |
| 7781 | 2P | BG13NUC-LC445 |
| 7782 | 2P | BG13NUC-LC446 |
| 7783 | 2P | BG13NUC-LC447 |
| 7784 | 2P | BG13NUC-LC448 |
| 7785 | RBD | BG13NUC-LC449 |
| 7786 | 2P | BG13NUC-LC450 |
| 7787 | 2P | BG13NUC-LC451 |
| 7788 | 2P | BG13NUC-LC452 |
| 7789 | 2P | BG13NUC-LC453 |
| 7790 | RBD | BG13NUC-LC454 |
| 7791 | RBD | BG13NUC-LC455 |
| 7792 | 2P | BG13NUC-LC456 |
| 7793 | 2P | BG13NUC-LC457 |
| 7794 | 2P | BG13NUC-LC458 |
| 7795 | RBD | BG13NUC-LC459 |
| 7796 | 2P | BG13NUC-LC460 |
| 7797 | RBD | BG13NUC-LC461 |
| 7798 | 2P | BG13NUC-LC462 |
| 7799 | RBD | BG13NUC-LC463 |
| 7800 | RBD | BG13NUC-LC464 |
| 7801 | 2P | BG13NUC-LC465 |
| 7802 | 2P | BG13NUC-LC466 |
| 7803 | 2P | BG13NUC-LC467 |
| 7804 | 2P | BG13NUC-LC468 |
| 7805 | 2P | BG13NUC-LC469 |
| 7806 | 2P | BG13NUC-LC470 |
| 7807 | RBD | BG13NUC-LC471 |
| 7808 | 2P | BG13NUC-LC472 |
| 7809 | RBD | BG13NUC-LC473 |
| 7810 | RBD | BG13NUC-LC474 |
| 7811 | 2P | BG13NUC-LC475 |
| 7812 | 2P | BG13NUC-LC476 |
| 7813 | 2P | BG13NUC-LC477 |
| 7814 | 2P | BG13NUC-LC478 |
| 7815 | 2P | BG13NUC-LC479 |
| 7816 | 2P | BG13NUC-LC480 |
| 7817 | 2P | BG13NUC-LC481 |
| 7818 | 2P | BG13NUC-LC482 |
| 7819 | 2P | BG13NUC-LC483 |
| 7820 | 2P | BG13NUC-LC484 |
| 7821 | 2P | BG13NUC-LC485 |
| 7822 | RBD | BG13NUC-LC486 |
| 7823 | RBD | BG13NUC-LC487 |
| 7824 | 2P | BG13NUC-LC488 |
| 7825 | 2P | BG13NUC-LC489 |
| 7826 | 2P | BG13NUC-LC490 |
| 7827 | 2P | BG13NUC-LC491 |
| 7828 | 2P | BG13NUC-LC492 |
| 7829 | 2P | BG13NUC-LC493 |
| 7830 | 2P | BG13NUC-LC494 |
| 7831 | 2P | BG13NUC-LC495 |
| 7832 | RBD | BG13NUC-LC496 |
| 7833 | RBD | BG13NUC-LC497 |
| 7834 | 2P | BG13NUC-LC498 |
| 7835 | RBD | BG13NUC-LC499 |
| 7836 | 2P | BG13NUC-LC500 |
| 7837 | RBD | BG13NUC-LC501 |
| 7838 | 2P | BG13NUC-LC502 |
| 7839 | 2P | BG13NUC-LC503 |
| 7840 | RBD | BG13NUC-LC504 |
| 7841 | 2P | BG13NUC-LC505 |
| 7842 | RBD | BG13NUC-LC506 |
| 7843 | RBD | BG13NUC-LC507 |
| 7844 | 2P | BG13NUC-LC508 |
| 7845 | 2P | BG13NUC-LC509 |
| 7846 | 2P | BG13NUC-LC510 |
| 7847 | 2P | BG13NUC-LC511 |
| 7848 | RBD | BG13NUC-LC512 |
| 7849 | RBD | BG13NUC-LC513 |
| 7850 | RBD | BG13NUC-LC514 |
| 7851 | 2P | BG13NUC-LC515 |
| 7852 | 2P | BG13NUC-LC516 |
| 7853 | 2P | BG13NUC-LC517 |
| 7854 | RBD | BG13NUC-LC518 |
| 7855 | RBD | BG13NUC-LC519 |
| 7856 | 2P | BG13NUC-LC520 |
| 7857 | 2P | BG13NUC-LC521 |
| 7858 | RBD | BG13NUC-LC522 |
| 7859 | RBD | BG13NUC-LC523 |
| 7860 | RBD | BG13NUC-LC524 |
| 7861 | 2P | BG13NUC-LC525 |
| 7862 | 2P | BG13NUC-LC526 |
| 7863 | 2P | BG13NUC-LC527 |
| 7864 | 2P | BG13NUC-LC528 |
| 7865 | 2P | BG13NUC-LC529 |
| 7866 | RBD | BG13NUC-LC530 |
| 7867 | 2P | BG13NUC-LC531 |
| 7868 | RBD | BG13NUC-LC532 |
| 7869 | RBD | BG13NUC-LC533 |
| 7870 | RBD | BG13NUC-LC534 |
| 7871 | RBD | BG13NUC-LC535 |
| 7872 | 2P | BG13NUC-LC536 |
| 7873 | 2P | BG13NUC-LC537 |
| 7874 | 2P | BG13NUC-LC538 |
| 7875 | 2P | BG13NUC-LC539 |
| 7876 | 2P | BG13NUC-LC540 |
| 7877 | 2P | BG13NUC-LC541 |
| 7878 | RBD | BG13NUC-LC542 |
| 7879 | RBD | BG13NUC-LC543 |
| 7880 | 2P | BG13NUC-LC544 |
| 7881 | 2P | BG13NUC-LC545 |
| 7882 | RBD | BG13NUC-LC546 |
| 7883 | RBD | BG13NUC-LC547 |
| 7884 | 2P | BG13NUC-LC548 |
| 7885 | RBD | BG13NUC-LC549 |
| 7886 | 2P | BG13NUC-LC550 |
| 7887 | 2P | BG13NUC-LC551 |
| 7888 | RBD | BG13NUC-LC552 |
| 7889 | RBD | BG13NUC-LC553 |
| 7890 | RBD | BG13NUC-LC554 |
| 7891 | RBD | BG13NUC-LC555 |
| 7892 | 2P | BG13NUC-LC556 |
| 7893 | 2P | BG13NUC-LC557 |
| 7894 | RBD | BG13NUC-LC558 |
| 7895 | 2P | BG13NUC-LC559 |
| 7896 | RBD | BG13NUC-LC560 |
| 7897 | 2P | BG13NUC-LC561 |
| 7898 | 2P | BG13NUC-LC562 |
| 7899 | 2P | BG13NUC-LC563 |
| 7900 | 2P | BG13NUC-LC564 |
| 7901 | 2P | BG13NUC-LC565 |
| 7902 | 2P | BG13NUC-LC566 |
| 7903 | 2P | BG13NUC-LC567 |
| 7904 | RBD | BG13NUC-LC568 |
| 7905 | RBD | BG13NUC-LC569 |
| 7906 | RBD | BG13NUC-LC570 |
| 7907 | RBD | BG13NUC-LC571 |
| 7908 | 2P | BG13NUC-LC572 |
| 7909 | 2P | BG13NUC-LC573 |
| 7910 | 2P | BG13NUC-LC574 |
| 7911 | 2P | BG13NUC-LC575 |
| 7912 | 2P | BG13NUC-LC576 |
| 7913 | 2P | BG13NUC-LC577 |
| 7914 | 2P | BG13NUC-LC578 |
| 7915 | 2P | BG13NUC-LC579 |
| 7916 | 2P | BG13NUC-LC580 |
| 7917 | 2P | BG13NUC-LC581 |
| 7918 | 2P | BG13NUC-LC582 |
| 7919 | 2P | BG13NUC-LC583 |
| 7920 | 2P | BG13NUC-LC584 |
| 7921 | 2P | BG13NUC-LC585 |
| 7922 | 2P | BG13NUC-LC586 |
| 7923 | 2P | BG13NUC-LC587 |
| 7924 | 2P | BG13NUC-LC588 |
| 7925 | 2P | BG13NUC-LC589 |
| 7926 | 2P | BG13NUC-LC590 |
| 7927 | RBD | BG13NUC-LC591 |
| 7928 | 2P | BG13NUC-LC592 |
| 7929 | RBD | BG13NUC-LC593 |
| 7930 | 2P | BG13NUC-LC594 |
| 7931 | RBD | BG13NUC-LC595 |
| 7932 | 2P | BG13NUC-LC596 |
| 7933 | 2P | BG13NUC-LC597 |
| 7934 | RBD | BG13NUC-LC598 |
| 7935 | 2P | BG13NUC-LC599 |
| 7936 | 2P | BG13NUC-LC600 |
| 7937 | 2P | BG13NUC-LC601 |
| 7938 | 2P | BG13NUC-LC602 |
| 7939 | 2P | BG13NUC-LC603 |
| 7940 | 2P | BG13NUC-LC604 |
| 7941 | 2P | BG13NUC-LC605 |
| 7942 | RBD | BG13NUC-LC606 |
| 7943 | RBD | BG13NUC-LC607 |
| 7944 | RBD | BG13NUC-LC608 |
| 7945 | 2P | BG13NUC-LC609 |
| 7946 | RBD | BG13NUC-LC610 |
| 7947 | 2P | BG13NUC-LC611 |
| 7948 | RBD | BG13NUC-LC612 |
| 7949 | RBD | BG13NUC-LC613 |
| 7950 | RBD | BG13NUC-LC614 |
| 7951 | 2P | BG13NUC-LC615 |
| 7952 | 2P | BG13NUC-LC616 |
| 7953 | RBD | BG13NUC-LC617 |
| 7954 | RBD | BG13NUC-LC618 |
| 7955 | 2P | BG13NUC-LC619 |
| 7956 | RBD | BG13NUC-LC620 |
| 7957 | 2P | BG13NUC-LC621 |
| 7958 | 2P | BG13NUC-LC622 |
| 7959 | RBD | BG13NUC-LC623 |
| 7960 | RBD | BG13NUC-LC624 |
| 7961 | 2P | BG13NUC-LC625 |
| 7962 | 2P | BG13NUC-LC626 |
| 7963 | 2P | BG13NUC-LC627 |
| 7964 | RBD | BG13NUC-LC628 |
| 7965 | RBD | BG13NUC-LC629 |
| 7966 | RBD | BG13NUC-LC630 |
| 7967 | 2P | BG13NUC-LC631 |
| 7968 | 2P | BG13NUC-LC632 |
| 7969 | 2P | BG13NUC-LC633 |
| 7970 | 2P | BG13NUC-LC634 |
| 7971 | 2P | BG13NUC-LC635 |
| 7972 | RBD | BG13NUC-LC636 |
| 7973 | RBD | BG13NUC-LC637 |
| 7974 | RBD | BG13NUC-LC638 |
| 7975 | 2P | BG13NUC-LC639 |
| 7976 | RBD | BG13NUC-LC640 |
| 7977 | RBD | BG13NUC-LC641 |
| 7978 | 2P | BG13NUC-LC642 |
| 7979 | 2P | BG13NUC-LC643 |
| 7980 | RBD | BG13NUC-LC644 |
| 7981 | RBD | BG13NUC-LC645 |
| 7982 | 2P | BG13NUC-LC646 |
| 7983 | RBD | BG13NUC-LC647 |
| 7984 | 2P | BG13NUC-LC648 |
| 7985 | 2P | BG13NUC-LC649 |
| 7986 | RBD | BG13NUC-LC650 |
| 7987 | RBD | BG13NUC-LC651 |
| 7988 | 2P | BG13NUC-LC652 |
| 7989 | 2P | BG13NUC-LC653 |
| 7990 | 2P | BG13NUC-LC654 |
| 7991 | 2P | BG13NUC-LC655 |
| 7992 | RBD | BG13NUC-LC656 |
| 7993 | RBD | BG13NUC-LC657 |
| 7994 | 2P | BG13NUC-LC658 |
| 7995 | 2P | BG13NUC-LC659 |
| 7996 | 2P | BG13NUC-LC660 |
| 7997 | 2P | BG13NUC-LC661 |
| 7998 | 2P | BG13NUC-LC662 |
| 7999 | 2P | BG13NUC-LC663 |
| 8000 | RBD | BG13NUC-LC664 |
| 8001 | 2P | BG13NUC-LC665 |
| 8002 | RBD | BG13NUC-LC666 |
| 8003 | 2P | BG13NUC-LC667 |
| 8004 | RBD | BG13NUC-LC668 |
| 8005 | RBD | BG13NUC-LC669 |
| 8006 | RBD | BG13NUC-LC670 |
| 8007 | 2F | BG13NUC-LC671 |
| 8008 | RBD | BG13NUC-LC672 |
| 8009 | 2P | BG13NUC-LC673 |
| 8010 | RBD | BG13NUC-LC674 |
| 8011 | RBD | BG13NUC-LC675 |
| 8012 | 2P | BG13NUC-LC676 |
| 8013 | 2P | BG13NUC-LC677 |
| 8014 | 2P | BG13NUC-LC678 |
| 8015 | RBD | BG13NUC-LC679 |
| 8016 | 2P | BG13NUC-LC680 |
| 8017 | 2P | BG13NUC-LC681 |
| 8018 | 2P | BG13NUC-LC682 |
| 8019 | 2P | BG13NUC-LC683 |
| 8020 | RBD | BG13NUC-LC684 |
| 8021 | 2P | BG13NUC-LC685 |
| 8022 | 2P | BG13NUC-LC686 |
| 8023 | 2P | BG13NUC-LC687 |
| 8024 | RBD | BG13NUC-LC688 |
| 8025 | 2P | BG13NUC-LC689 |
| 8026 | RBD | BG13NUC-LC690 |
| 8027 | 2F | BG13NUC-LC691 |
| 8028 | RBD | BG13NUC-LC692 |
| 8029 | RBD | BG13NUC-LC693 |
| 8030 | RBD | BG13NUC-LC694 |
| 8031 | 2P | BG13NUC-LC695 |
| 8032 | RBD | BG13NUC-LC696 |
| 8033 | RBD | BG13NUC-LC697 |
| 8034 | RBD | BG13NUC-LC698 |
| 8035 | 2P | BG13NUC-LC699 |
| 8036 | RBD | BG13NUC-LC700 |
| 8037 | RBD | BG13NUC-LC701 |
| 8038 | 2P | BG13NUC-LC702 |
| 8039 | 2P | BG13NUC-LC703 |
| 8040 | 2P | BG13NUC-LC704 |
| 8041 | 2P | BG13NUC-LC705 |
| 8042 | 2P | BG13NUC-LC706 |
| 8043 | 2P | BG13NUC-LC707 |
| 8044 | 2P | BG13NUC-LC708 |
| 8045 | 2P | BG13NUC-LC709 |
| 8046 | 2P | BG13NUC-LC710 |
| 8047 | 2P | BG13NUC-LC711 |
| 8048 | 2P | BG13NUC-LC712 |
| 8049 | 2P | BG13NUC-LC713 |
| 8050 | 2P | BG13NUC-LC714 |
| 8051 | RBD | BG13NUC-LC715 |
| 8052 | RBD | BG13NUC-LC716 |
| 8053 | RBD | BG13NUC-LC717 |
| 8054 | RBD | BG13NUC-LC718 |
| 8055 | RBD | BG13NUC-LC719 |
| 8056 | 2P | BG13NUC-LC720 |
| 8057 | 2P | BG13NUC-LC721 |
| 8058 | 2P | BG13NUC-LC722 |
| 8059 | 2P | BG13NUC-LC723 |
| 8060 | 2P | BG13NUC-LC724 |
| 8061 | 2P | BG13NUC-LC725 |
| 8062 | 2P | BG13NUC-LC726 |
| 8063 | 2P | BG13NUC-LC727 |
| 8064 | 2P | BG13NUC-LC728 |
| 8065 | RBD | BG13NUC-LC729 |
| 8066 | 2P | BG13NUC-LC730 |
| 8067 | 2P | BG13NUC-LC731 |
| 8068 | 2P | BG13NUC-LC732 |
| 8069 | 2P | BG13NUC-LC733 |
| 8070 | 2P | BG13NUC-LC734 |
| 8071 | 2P | BG13NUC-LC735 |
| 8072 | 2P | BG13NUC-LC736 |
| 8073 | RBD | BG13NUC-LC737 |
| 8074 | 2P | BG13NUC-LC738 |
| 8075 | RBD | BG13NUC-LC739 |
| 8076 | RBD | BG13NUC-LC740 |
| 8077 | RBD | BG13NUC-LC741 |
| 8078 | 2P | BG13NUC-LC742 |
| 8079 | 2P | BG13NUC-LC743 |
| 8080 | 2P | BG13NUC-LC744 |
| 8081 | RBD | BG13NUC-LC745 |
| 8082 | 2F | BG13NUC-LC746 |
| 8083 | RBD | BG13NUC-LC747 |
| 8084 | 2P | BG13NUC-LC748 |
| 8085 | 2P | BG13NUC-LC749 |
| 8086 | 2P | BG13NUC-LC750 |
| 8087 | 2P | BG13NUC-LC751 |
| 8088 | 2P | BG13NUC-LC752 |
| 8089 | 2P | BG13NUC-LC753 |
| 8090 | 2P | BG13NUC-LC754 |
| 8091 | 2P | BG13NUC-LC755 |
| 8092 | 2P | BG13NUC-LC756 |
| 8093 | 2P | BG13NUC-LC757 |
| 8094 | RBD | BG13NUC-LC758 |
| 8095 | 2P | BG13NUC-LC759 |
| 8096 | 2P | BG13NUC-LC760 |
| 8097 | 2P | BG13NUC-LC761 |
| 8098 | 2P | BG13NUC-LC762 |
| 8099 | RBD | BG13NUC-LC763 |
| 8100 | 2P | BG13NUC-LC764 |
| 8101 | 2P | BG13NUC-LC765 |
| 8102 | 2P | BG13NUC-LC766 |
| 8103 | 2P | BG13NUC-LC767 |
| 8104 | 2P | BG13NUC-LC768 |
| 8105 | 2P | BG13NUC-LC769 |
| 8106 | 2P | BG13NUC-LC770 |
| 8107 | 2P | BG13NUC-LC771 |
| 8108 | 2P | BG13NUC-LC772 |
| 8109 | RBD | BG13NUC-LC773 |
| 8110 | RBD | BG13NUC-LC774 |
| 8111 | RBD | BG13NUC-LC775 |
| 8112 | RBD | BG13NUC-LC776 |
| 8113 | RBD | BG13NUC-LC777 |
| 8114 | RBD | BG13NUC-LC778 |
| 8115 | 2P | BG13NUC-LC779 |
| 8116 | RBD | BG13NUC-LC780 |
| 8117 | RBD | BG13NUC-LC781 |
| 8118 | 2P | BG13NUC-LC782 |
| 8119 | 2P | BG13NUC-LC783 |
| 8120 | RBD | BG13NUC-LC784 |
| 8121 | RBD | BG13NUC-LC785 |
| 8122 | 2P | BG13NUC-LC786 |
| 8123 | 2P | BG13NUC-LC787 |
| 8124 | 2P | BG13NUC-LC788 |
| 8125 | 2P | BG13NUC-LC789 |
| 8126 | RBD | BG13NUC-LC790 |
| 8127 | 2P | BG13NUC-LC791 |
| 8128 | RBD | BG13NUC-LC792 |
| 8129 | RBD | BG13NUC-LC793 |
| 8130 | 2P | BG13NUC-LC794 |
| 8131 | 2P | BG13NUC-LC795 |
| 8132 | 2P | BG13NUC-LC796 |
| 8133 | 2P | BG13NUC-LC797 |
| 8134 | 2P | BG13NUC-LC798 |
| 8135 | RBD | BG13NUC-LC799 |
| 8136 | RBD | BG13NUC-LC800 |
| 8137 | RBD | BG13NUC-LC801 |
| 8138 | 2P | BG13NUC-LC802 |
| 8139 | 2P | BG13NUC-LC803 |
| 8140 | 2P | BG13NUC-LC804 |
| 8141 | 2P | BG13NUC-LC805 |
| 8142 | 2P | BG13NUC-LC806 |
| 8143 | 2P | BG13NUC-LC807 |
| 8144 | 2P | BG13NUC-LC808 |
| 8145 | 2P | BG13NUC-LC809 |
| 8146 | 2P | BG13NUC-LC810 |
| 8147 | RBD | BG13NUC-LC811 |
| 8148 | RBD | BG13NUC-LC812 |
| 8149 | RBD | BG13NUC-LC813 |
| 8150 | 2P | BG13NUC-LC814 |
| 8151 | 2P | BG13NUC-LC815 |
| 8152 | 2P | BG13NUC-LC816 |
| 8153 | 2P | BG13NUC-LC817 |
| 8154 | 2P | BG13NUC-LC818 |
| 8155 | 2P | BG13NUC-LC819 |
| 8156 | 2P | BG13NUC-LC820 |
| 8157 | 2P | BG13NUC-LC821 |
| 8158 | 2P | BG13NUC-LC822 |
| 8159 | RBD | BG13NUC-LC823 |
| 8160 | RBD | BG13NUC-LC824 |
| 8161 | RBD | BG13NUC-LC825 |
| 8162 | RBD | BG13NUC-LC826 |
| 8163 | RBD | BG13NUC-LC827 |
| 8164 | 2P | BG13NUC-LC828 |
| 8165 | 2P | BG13NUC-LC829 |
| 8166 | 2P | BG13NUC-LC830 |
| 8167 | 2P | BG13NUC-LC831 |
| 8168 | 2P | BG13NUC-LC832 |
| 8169 | RBD | BG13NUC-LC833 |
| 8170 | RBD | BG13NUC-LC834 |
| 8171 | RBD | BG13NUC-LC835 |
| 8172 | 2P | BG13NUC-LC836 |
| 8173 | RBD | BG13NUC-LC837 |
| 8174 | 2P | BG13NUC-LC838 |
| 8175 | RBD | BG13NUC-LC839 |
| 8176 | 2P | BG13NUC-LC840 |
| 8177 | 2P | BG13NUC-LC841 |
| 8178 | RBD | BG13NUC-LC842 |
| 8179 | RBD | BG13NUC-LC843 |
| 8180 | 2P | BG13NUC-LC844 |
| 8181 | RBD | BG13NUC-LC845 |
| 8182 | RBD | BG13NUC-LC846 |
| 8183 | 2P | BG13NUC-LC847 |
| 8184 | 2P | BG13NUC-LC848 |
| 8185 | RBD | BG13NUC-LC849 |
| 8186 | RBD | BG13NUC-LC850 |
| 8187 | RBD | BG13NUC-LC851 |
| 8188 | 2P | BG13NUC-LC852 |
| 8189 | 2P | BG13NUC-LC853 |
| 8190 | 2P | BG13NUC-LC854 |
| 8191 | 2P | BG13NUC-LC855 |
| 8192 | 2P | BG13NUC-LC856 |
| 8193 | RBD | BG13NUC-LC857 |
| 8194 | 2P | BG13NUC-LC858 |
| 8195 | 2P | BG13NUC-LC859 |
| 8196 | 2P | BG13NUC-LC860 |
| 8197 | 2P | BG13NUC-LC861 |
| 8198 | 2P | BG13NUC-LC862 |
| 8199 | RBD | BG13NUC-LC863 |
| 8200 | 2P | BG13NUC-LC864 |
| 8201 | 2P | BG13NUC-LC865 |
| 8202 | 2P | BG13NUC-LC866 |
| 8203 | 2P | BG13NUC-LC867 |
| 8204 | 2F | BG13NUC-LC868 |
| 8205 | 2P | BG13NUC-LC869 |
| 8206 | RBD | BG13NUC-LC870 |
| 8207 | 2P | BG13NUC-LC871 |
| 8208 | RBD | BG13NUC-LC872 |
| 8209 | RBD | BG13NUC-LC873 |
| 8210 | RBD | BG13NUC-LC874 |
| 8211 | RBD | BG13NUC-LC875 |
| 8212 | RBD | BG13NUC-LC876 |
| 8213 | RBD | BG13NUC-LC877 |
| 8214 | 2P | BG13NUC-LC878 |
| 8215 | 2P | BG13NUC-LC879 |
| 8216 | 2P | BG13NUC-LC880 |
| 8217 | 2P | BG13NUC-LC881 |
| 8218 | RBD | BG13NUC-LC882 |
| 8219 | 2P | BG13NUC-LC883 |
| 8220 | RBD | BG13NUC-LC884 |
| 8221 | RBD | BG13NUC-LC885 |
| 8222 | 2P | BG13NUC-LC886 |
| 8223 | RBD | BG13NUC-LC887 |
| 8224 | RBD | BG13NUC-LC888 |
| 8225 | RBD | BG13NUC-LC889 |
| 8226 | RBD | BG13NUC-LC890 |
| 8227 | RBD | BG13NUC-LC891 |
| 8228 | RBD | BG13NUC-LC892 |
| 8229 | 2P | BG13NUC-LC893 |
| 8230 | RBD | BG13NUC-LC894 |
| 8231 | RBD | BG13NUC-LC895 |
| 8232 | RBD | BG13NUC-LC896 |
| 8233 | RBD | BG13NUC-LC897 |
| 8234 | RBD | BG13NUC-LC898 |
| 8235 | RBD | BG13NUC-LC899 |
| 8236 | 2P | BG13NUC-LC900 |
| 8237 | 2P | BG13NUC-LC901 |
| 8238 | 2P | BG13NUC-LC902 |
| 8239 | 2P | BG13NUC-LC903 |
| 8240 | 2P | BG13NUC-LC904 |
| 8241 | RBD | BG13NUC-LC905 |
| 8242 | 2P | BG13NUC-LC906 |
| 8243 | RBD | BG13NUC-LC907 |
| 8244 | 2P | BG13NUC-LC908 |
| 8245 | 2P | BG13NUC-LC909 |
| 8246 | 2P | BG13NUC-LC910 |
| 8247 | 2P | BG13NUC-LC911 |
| 8248 | 2P | BG13NUC-LC912 |
| 8249 | 2P | BG13NUC-LC913 |
| 8250 | 2P | BG13NUC-LC914 |
| 8251 | 2P | BG13NUC-LC915 |
| 8252 | 2P | BG13NUC-LC916 |
| 8253 | 2P | BG13NUC-LC917 |
| 8254 | 2P | BG13NUC-LC918 |
| 8255 | RBD | BG13NUC-LC919 |
| 8256 | 2P | BG13NUC-LC920 |
| 8257 | RBD | BG13NUC-LC921 |
| 8258 | RBD | BG13NUC-LC922 |
| 8259 | RBD | BG13NUC-LC923 |
| 8260 | RBD | BG13NUC-LC924 |
| 8261 | 2P | BG13NUC-LC925 |
| 8262 | 2P | BG13NUC-LC926 |
| 8263 | RBD | BG13NUC-LC927 |
| 8264 | 2P | BG13NUC-LC928 |
| 8265 | 2P | BG13NUC-LC929 |
| 8266 | 2P | BG13NUC-LC930 |
| 8267 | 2P | BG13NUC-LC931 |
| 8268 | 2P | BG13NUC-LC932 |
| 8269 | 2P | BG13NUC-LC933 |
| 8270 | 2P | BG13NUC-LC934 |
| 8271 | RBD | BG13NUC-LC935 |
| 8272 | RBD | BG13NUC-LC936 |
| 8273 | 2P | BG13NUC-LC937 |
| 8274 | 2P | BG13NUC-LC938 |
| 8275 | 2P | BG13NUC-LC939 |
| 8276 | RBD | BG13NUC-LC940 |
| 8277 | 2P | BG13NUC-LC941 |
| 8278 | RBD | BG13NUC-LC942 |
| 8279 | RBD | BG13NUC-LC943 |
| 8280 | RBD | BG13NUC-LC944 |
| 8281 | RBD | BG13NUC-LC945 |
| 8282 | 2P | BG13NUC-LC946 |
| 8283 | RBD | BG13NUC-LC947 |
| 8284 | RBD | BG13NUC-LC948 |
| 8285 | RBD | BG13NUC-LC949 |
| 8286 | 2P | BG13NUC-LC950 |
| 8287 | RBD | BG13NUC-LC951 |
| 8288 | 2P | BG13NUC-LC952 |
| 8289 | 2P | BG13NUC-LC953 |
| 8290 | 2P | BG13NUC-LC954 |
| 8291 | 2P | BG13NUC-LC955 |
| 8292 | 2P | BG13NUC-LC956 |
| 8293 | RBD | BG13NUC-LC957 |
| 8294 | 2P | BG13NUC-LC958 |
| 8295 | RBD | BG13NUC-LC959 |
| 8296 | RBD | BG13NUC-LC960 |
| 8297 | 2P | BG13NUC-LC961 |
| 8298 | RBD | BG13NUC-LC962 |
| 8299 | RBD | BG13NUC-LC963 |
| 8300 | 2P | BG13NUC-LC964 |
| 8301 | RBD | BG13NUC-LC965 |
| 8302 | 2P | BG13NUC-LC966 |
| 8303 | 2P | BG13NUC-LC967 |
| 8304 | 2P | BG13NUC-LC968 |
| 8305 | 2P | BG13NUC-LC969 |
| 8306 | RBD | BG13NUC-LC970 |
| 8307 | 2P | BG13NUC-LC971 |
| 8308 | RBD | BG13NUC-LC972 |
| 8309 | 2P | BG13NUC-LC973 |
| 8310 | RBD | BG13NUC-LC974 |
| 8311 | RBD | BG13NUC-LC975 |
| 8312 | RBD | BG13NUC-LC976 |
| 8313 | 2P | BG13NUC-LC977 |
| 8314 | RBD | BG13NUC-LC978 |
| 8315 | 2P | BG13NUC-LC979 |
| 8316 | 2P | BG13NUC-LC980 |
| 8317 | RBD | BG13NUC-LC981 |
| 8318 | 2P | BG13NUC-LC982 |
| 8319 | 2P | BG13NUC-LC983 |
| 8320 | RBD | BG13NUC-LC984 |
| 8321 | RBD | BG13NUC-LC985 |
| 8322 | RBD | BG13NUC-LC986 |
| 8323 | RBD | BG13NUC-LC987 |
| 8324 | 2P | BG13NUC-LC988 |
| 8325 | 2P | BG13NUC-LC989 |
| 8326 | 2P | BG13NUC-LC990 |
| 8327 | RBD | BG13NUC-LC991 |
| 8328 | 2P | BG13NUC-LC992 |
| 8329 | 2P | BG13NUC-LC993 |
| 8330 | RBD | BG13NUC-LC994 |
| 8331 | RBD | BG13NUC-LC995 |
| 8332 | 2P | BG13NUC-LC996 |
| 8333 | RBD | BG13NUC-LC997 |
| 8334 | 2P | BG13NUC-LC998 |
| 8335 | RBD | BG13NUC-LC999 |
| 8336 | 2P | BG13NUC-LC1000 |
| 8337 | RBD | BG13NUC-LC1001 |
| 8338 | RBD | BG13NUC-LC1002 |
| 8339 | 2P | BG13NUC-LC1003 |
| 8340 | RBD | BG13NUC-LC1004 |
| 8341 | 2P | BG13NUC-LC1005 |
| 8342 | 2P | BG13NUC-LC1006 |
| 8343 | 2P | BG13NUC-LC1007 |
| 8344 | 2P | BG13NUC-LC1008 |
| 8345 | 2P | BG13NUC-LC1009 |
| 8346 | RBD | BG13NUC-LC1010 |
| 8347 | 2P | BG13NUC-LC1011 |
| 8348 | 2P | BG13NUC-LC1012 |
| 8349 | RBD | BG13NUC-LC1013 |
| 8350 | RBD | BG13NUC-LC1014 |
| 8351 | 2P | BG13NUC-LC1015 |
| 8352 | 2P | BG13NUC-LC1016 |
| 8353 | 2P | BG13NUC-LC1017 |
| 8354 | 2P | BG13NUC-LC1018 |
| 8355 | 2P | BG13NUC-LC1019 |
| 8356 | 2P | BG13NUC-LC1020 |
| 8357 | 2P | BG13NUC-LC1021 |
| 8358 | RBD | BG13NUC-LC1022 |
| 8359 | 2P | BG13NUC-LC1023 |
| 8360 | 2P | BG13NUC-LC1024 |
| 8361 | 2P | BG13NUC-LC1025 |
| 8362 | RBD | BG13NUC-LC1026 |
| 8363 | 2P | BG13NUC-LC1027 |
| 8364 | 2P | BG13NUC-LC1028 |
| 8365 | 2P | BG13NUC-LC1029 |
| 8366 | 2P | BG13NUC-LC1030 |
| 8367 | 2P | BG13NUC-LC1031 |
| 8368 | RBD | BG13NUC-LC1032 |
| 8369 | RBD | BG13NUC-LC1033 |
| 8370 | 2P | BG13NUC-LC1034 |
| 8371 | 2P | BG13NUC-LC1035 |
| 8372 | 2P | BG13NUC-LC1036 |
| 8373 | 2P | BG13NUC-LC1037 |
| 8374 | RBD | BG13NUC-LC1038 |
| 8375 | RBD | BG13NUC-LC1039 |
| 8376 | RBD | BG13NUC-LC1040 |
| 8377 | 2P | BG13NUC-LC1041 |
| 8378 | 2F | BG13NUC-LC1042 |
| 8379 | RBD | BG13NUC-LC1043 |
| 8380 | 2P | BG13NUC-LC1044 |
| 8381 | 2P | BG13NUC-LC1045 |
| 8382 | 2P | BG13NUC-LC1046 |
| 8383 | 2P | BG13NUC-LC1047 |
| 8384 | 2P | BG13NUC-LC1048 |
| 8385 | RBD | BG13NUC-LC1049 |
| 8386 | 2P | BG13NUC-LC1050 |
| 8387 | 2P | BG13NUC-LC1051 |
| 8388 | 2P | BG13NUC-LC1052 |
| 8389 | RBD | BG13NUC-LC1053 |
| 8390 | 2P | BG13NUC-LC1054 |
| 8391 | 2P | BG13NUC-LC1055 |
| 8392 | 2P | BG13NUC-LC1056 |
| 8393 | 2P | BG13NUC-LC1057 |
| 8394 | 2P | BG13NUC-LC1058 |
| 8395 | RBD | BG13NUC-LC1059 |
| 8396 | 2P | BG13NUC-LC1060 |
| 8397 | RBD | BG13NUC-LC1061 |
| 8398 | 2P | BG13NUC-LC1062 |
| 8399 | 2P | BG13NUC-LC1063 |
| 8400 | 2P | BG13NUC-LC1064 |
| 8401 | RBD | BG13NUC-LC1065 |
| 8402 | 2P | BG13NUC-LC1066 |
| 8403 | 2P | BG13NUC-LC1067 |
| 8404 | RBD | BG13NUC-LC1068 |
| 8405 | 2P | BG13NUC-LC1069 |
| 8406 | 2P | BG13NUC-LC1070 |
| 8407 | 2P | BG13NUC-LC1071 |
| 8408 | RBD | BG13NUC-LC1072 |
| 8409 | RBD | BG13NUC-LC1073 |
| 8410 | 2P | BG13NUC-LC1074 |
| 8411 | RBD | BG13NUC-LC1075 |
| 8412 | 2P | BG13NUC-LC1076 |
| 8413 | 2F | BG13NUC-LC1077 |
| 8414 | RBD | BG13NUC-LC1078 |
| 8415 | RBD | BG13NUC-LC1079 |
| 8416 | RBD | BG13NUC-LC1080 |
| 8417 | RBD | BG13NUC-LC1081 |
| 8418 | 2P | BG13NUC-LC1082 |
| 8419 | 2P | BG13NUC-LC1083 |
| 8420 | RBD | BG13NUC-LC1084 |
| 8421 | 2P | BG13NUC-LC1085 |
| 8422 | RBD | BG13NUC-LC1086 |
| 8423 | 2P | BG13NUC-LC1087 |
| 8424 | 2P | BG13NUC-LC1088 |
| 8425 | 2P | BG13NUC-LC1089 |
| 8426 | RBD | BG13NUC-LC1090 |
| 8427 | RBD | BG13NUC-LC1091 |
| 8428 | 2P | BG13NUC-LC1092 |
| 8429 | RBD | BG13NUC-LC1093 |
| 8430 | 2P | BG13NUC-LC1094 |
| 8431 | RBD | BG13NUC-LC1095 |
| 8432 | RBD | BG13NUC-LC1096 |
| 8433 | RBD | BG13NUC-LC1097 |
| 8434 | RBD | BG13NUC-LC1098 |
| 8435 | 2P | BG13NUC-LC1099 |
| 8436 | RBD | BG13NUC-LC1100 |
| 8437 | RBD | BG13NUC-LC1101 |
| 8438 | RBD | BG13NUC-LC1102 |
| 8439 | RBD | BG13NUC-LC1103 |
| 8440 | 2P | BG13NUC-LC1104 |
| 8441 | 2P | BG13NUC-LC1105 |
| 8442 | RBD | BG13NUC-LC1106 |
| 8443 | 2P | BG13NUC-LC1107 |
| 8444 | RBD | BG13NUC-LC1108 |
| 8445 | 2P | BG13NUC-LC1109 |
| 8446 | 2P | BG13NUC-LC1110 |
| 8447 | 2P | BG13NUC-LC1111 |
| 8448 | RBD | BG13NUC-LC1112 |
| 8449 | 2P | BG13NUC-LC1113 |
| 8450 | RBD | BG13NUC-LC1114 |
| 8451 | 2P | BG13NUC-LC1115 |
| 8452 | 2P | BG13NUC-LC1116 |
| 8453 | 2P | BG13NUC-LC1117 |
| 8454 | 2P | BG13NUC-LC1118 |
| 8455 | 2P | BG13NUC-LC1119 |
| 8456 | 2P | BG13NUC-LC1120 |
| 8457 | 2P | BG13NUC-LC1121 |
| 8458 | 2P | BG13NUC-LC1122 |
| 8459 | 2P | BG13NUC-LC1123 |
| 8460 | 2P | BG13NUC-LC1124 |
| 8461 | 2F | BG13NUC-LC1125 |
| 8462 | RBD | BG13NUC-LC1126 |
| 8463 | 2P | BG13NUC-LC1127 |
| 8464 | 2P | BG13NUC-LC1128 |
| 8465 | 2P | BG13NUC-LC1129 |
| 8466 | 2P | BG13NUC-LC1130 |
| 8467 | RBD | BG13NUC-LC1131 |
| 8468 | RBD | BG13NUC-LC1132 |
| 8469 | 2P | BG13NUC-LC1133 |
| 8470 | RBD | BG13NUC-LC1134 |
| 8471 | RBD | BG13NUC-LC1135 |
| 8472 | RBD | BG13NUC-LC1136 |
| 8473 | RBD | BG14NUC-HC1 |
| 8474 | RBD | BG14NUC-HC2 |
| 8475 | RBD | BG14NUC-HC3 |
| 8476 | RBD | BG14NUC-HC4 |
| 8477 | RBD | BG14NUC-HC5 |
| 8478 | RBD | BG14NUC-HC6 |
| 8479 | RBD | BG14NUC-HC7 |
| 8480 | RBD | BG14NUC-HC8 |
| 8481 | RBD | BG14NUC-HC9 |
| 8482 | RBD | BG14NUC-HC10 |
| 8483 | RBD | BG14NUC-HC11 |
| 8484 | RBD | BG14NUC-HC12 |
| 8485 | RBD | BG14NUC-HC13 |
| 8486 | RBD | BG14NUC-HC14 |
| 8487 | RBD | BG14NUC-HC15 |
| 8488 | RBD | BG14NUC-HC16 |
| 8489 | RBD | BG14NUC-HC17 |
| 8490 | RBD | BG14NUC-HC18 |
| 8491 | RBD | BG14NUC-HC19 |
| 8492 | RBD | BG14NUC-HC20 |
| 8493 | RBD | BG14NUC-HC21 |
| 8494 | RBD | BG14NUC-HC22 |
| 8495 | RBD | BG14NUC-HC23 |
| 8496 | RBD | BG14NUC-HC24 |
| 8497 | RBD | BG14NUC-HC25 |
| 8498 | 2P | BG14NUC-HC26 |
| 8499 | 2P | BG14NUC-HC27 |
| 8500 | 2P | BG14NUC-HC28 |
| 8501 | 2P | BG14NUC-HC29 |
| 8502 | 2P | BG14NUC-HC30 |
| 8503 | 2P | BG14NUC-HC31 |
| 8504 | 2P | BG14NUC-HC32 |
| 8505 | 2P | BG14NUC-HC33 |
| 8506 | 2P | BG14NUC-HC34 |
| 8507 | 2P | BG14NUC-HC35 |
| 8508 | 2P | BG14NUC-HC36 |
| 8509 | 2P | BG14NUC-HC37 |
| 8510 | 2P | BG14NUC-HC38 |
| 8511 | 2P | BG14NUC-HC39 |
| 8512 | 2P | BG14NUC-HC40 |
| 8513 | 2P | BG14NUC-HC41 |
| 8514 | 2P | BG14NUC-HC42 |
| 8515 | 2P | BG14NUC-HC43 |
| 8516 | 2P | BG14NUC-HC44 |
| 8517 | 2P | BG14NUC-HC45 |
| 8518 | 2P | BG14NUC-HC46 |
| 8519 | 2P | BG14NUC-HC47 |
| 8520 | 2P | BG14NUC-HC48 |
| 8521 | 2P | BG14NUC-HC49 |
| 8522 | RBD | BG14NUC-HC50 |
| 8523 | RBD | BG14NUC-HC51 |
| 8524 | RBD | BG14NUC-HC52 |
| 8525 | RBD | BG14NUC-HC53 |
| 8526 | RBD | BG14NUC-HC54 |
| 8527 | RBD | BG14NUC-HC55 |
| 8528 | RBD | BG14NUC-HC56 |
| 8529 | RBD | BG14NUC-HC57 |
| 8530 | RBD | BG14NUC-HC58 |
| 8531 | 2P | BG14NUC-HC59 |
| 8532 | 2P | BG14NUC-HC60 |
| 8533 | 2P | BG14NUC-HC61 |
| 8534 | 2P | BG14NUC-HC62 |
| 8535 | 2P | BG14NUC-HC63 |
| 8536 | 2P | BG14NUC-HC64 |
| 8537 | 2P | BG14NUC-HC65 |
| 8538 | RBD | BG14NUC-HC66 |
| 8539 | RBD | BG14NUC-HC67 |
| 8540 | RBD | BG14NUC-HC68 |
| 8541 | RBD | BG14NUC-HC69 |
| 8542 | RBD | BG14NUC-HC70 |
| 8543 | 2F | BG14NUC-HC71 |
| 8544 | 2P | BG14NUC-HC72 |
| 8545 | RBD | BG14NUC-HC73 |
| 8546 | RBD | BG14NUC-HC74 |
| 8547 | RBD | BG14NUC-HC75 |
| 8548 | 2P | BG14NUC-HC76 |
| 8549 | RBD | BG14NUC-HC77 |
| 8550 | RBD | BG14NUC-HC78 |
| 8551 | RBD | BG14NUC-HC79 |
| 8552 | RBD | BG14NUC-HC80 |
| 8553 | RBD | BG14NUC-HC81 |
| 8554 | RBD | BG14NUC-HC82 |
| 8555 | RBD | BG14NUC-HC83 |
| 8556 | RBD | BG14NUC-HC84 |
| 8557 | RBD | BG14NUC-HC85 |
| 8558 | 2P | BG14NUC-HC86 |
| 8559 | 2P | BG14NUC-HC87 |
| 8560 | 2P | BG14NUC-HC88 |
| 8561 | 2F | BG14NUC-HC89 |
| 8562 | RBD | BG14NUC-HC90 |
| 8563 | RBD | BG14NUC-HC91 |
| 8564 | RBD | BG14NUC-HC92 |
| 8565 | 2P | BG14NUC-HC93 |
| 8566 | 2P | BG14NUC-HC94 |
| 8567 | 2P | BG14NUC-HC95 |
| 8568 | RBD | BG14NUC-HC96 |
| 8569 | RBD | BG14NUC-HC97 |
| 8570 | RBD | BG14NUC-HC98 |
| 8571 | 2P | BG14NUC-HC99 |
| 8572 | RBD | BG14NUC-HC100 |
| 8573 | RBD | BG14NUC-HC101 |
| 8574 | RBD | BG14NUC-HC102 |
| 8575 | 2P | BG14NUC-HC103 |
| 8576 | RBD | BG14NUC-HC104 |
| 8577 | RBD | BG14NUC-HC105 |
| 8578 | RBD | BG14NUC-HC106 |
| 8579 | RBD | BG14NUC-HC107 |
| 8580 | RBD | BG14NUC-HC108 |
| 8581 | 2P | BG14NUC-HC109 |
| 8582 | 2F | BG14NUC-HC110 |
| 8583 | RBD | BG14NUC-HC111 |
| 8584 | RBD | BG14NUC-HC112 |
| 8585 | 2P | BG14NUC-HC113 |
| 8586 | 2P | BG14NUC-HC114 |
| 8587 | 2P | BG14NUC-HC115 |
| 8588 | RBD | BG14NUC-HC116 |
| 8589 | RBD | BG14NUC-HC117 |
| 8590 | RBD | BG14NUC-HC118 |
| 8591 | RBD | BG14NUC-HC119 |
| 8592 | RBD | BG14NUC-HC120 |
| 8593 | 2P | BG14NUC-HC121 |
| 8594 | 2P | BG14NUC-HC122 |
| 8595 | RBD | BG14NUC-HC123 |
| 8596 | RBD | BG14NUC-HC124 |
| 8597 | RBD | BG14NUC-HC125 |
| 8598 | 2P | BG14NUC-HC126 |
| 8599 | 2P | BG14NUC-HC127 |
| 8600 | RBD | BG14NUC-HC128 |
| 8601 | RBD | BG14NUC-HC129 |
| 8602 | RBD | BG14NUC-HC130 |
| 8603 | 2P | BG14NUC-HC131 |
| 8604 | 2P | BG14NUC-HC132 |
| 8605 | RBD | BG14NUC-HC133 |
| 8606 | RBD | BG14NUC-HC134 |
| 8607 | RBD | BG14NUC-HC135 |
| 8608 | RBD | BG14NUC-HC136 |
| 8609 | RBD | BG14NUC-HC137 |
| 8610 | RBD | BG14NUC-HC138 |
| 8611 | RBD | BG14NUC-HC139 |
| 8612 | RBD | BG14NUC-HC140 |
| 8613 | 2P | BG14NUC-HC141 |
| 8614 | 2P | BG14NUC-HC142 |
| 8615 | RBD | BG14NUC-HC143 |
| 8616 | 2P | BG14NUC-HC144 |
| 8617 | 2P | BG14NUC-HC145 |
| 8618 | 2P | BG14NUC-HC146 |
| 8619 | RBD | BG14NUC-HC147 |
| 8620 | RBD | BG14NUC-HC148 |
| 8621 | 2P | BG14NUC-HC149 |
| 8622 | 2P | BG14NUC-HC150 |
| 8623 | 2P | BG14NUC-HC151 |
| 8624 | 2P | BG14NUC-HC152 |
| 8625 | 2F | BG14NUC-HC153 |
| 8626 | RBD | BG14NUC-HC154 |
| 8627 | 2P | BG14NUC-HC155 |
| 8628 | 2P | BG14NUC-HC156 |
| 8629 | RBD | BG14NUC-HC157 |
| 8630 | 2P | BG14NUC-HC158 |
| 8631 | 2P | BG14NUC-HC159 |
| 8632 | 2P | BG14NUC-HC160 |
| 8633 | 2P | BG14NUC-HC161 |
| 8634 | 2P | BG14NUC-HC162 |
| 8635 | RBD | BG14NUC-HC163 |
| 8636 | RBD | BG14NUC-HC164 |
| 8637 | RBD | BG14NUC-HC165 |
| 8638 | 2P | BG14NUC-HC166 |
| 8639 | 2P | BG14NUC-HC167 |
| 8640 | 2P | BG14NUC-HC168 |
| 8641 | 2P | BG14NUC-HC169 |
| 8642 | RBD | BG14NUC-HC170 |
| 8643 | 2P | BG14NUC-HC171 |
| 8644 | RBD | BG14NUC-HC172 |
| 8645 | 2P | BG14NUC-HC173 |
| 8646 | 2P | BG14NUC-HC174 |
| 8647 | 2P | BG14NUC-HC175 |
| 8648 | RBD | BG14NUC-HC176 |
| 8649 | 2P | BG14NUC-HC177 |
| 8650 | RBD | BG14NUC-HC178 |
| 8651 | 2P | BG14NUC-HC179 |
| 8652 | 2P | BG14NUC-HC180 |
| 8653 | 2P | BG14NUC-HC181 |
| 8654 | 2P | BG14NUC-HC182 |
| 8655 | 2P | BG14NUC-HC183 |
| 8656 | RBD | BG14NUC-HC184 |
| 8657 | 2P | BG14NUC-HC185 |
| 8658 | 2P | BG14NUC-HC186 |
| 8659 | 2P | BG14NUC-HC187 |
| 8660 | RBD | BG14NUC-HC188 |
| 8661 | 2P | BG14NUC-HC189 |
| 8662 | 2P | BG14NUC-HC190 |
| 8663 | 2P | BG14NUC-HC191 |
| 8664 | RBD | BG14NUC-HC192 |
| 8665 | RBD | BG14NUC-HC193 |
| 8666 | RBD | BG14NUC-HC194 |
| 8667 | RBD | BG14NUC-HC195 |
| 8668 | RBD | BG14NUC-HC196 |
| 8669 | 2P | BG14NUC-HC197 |
| 8670 | 2P | BG14NUC-HC198 |
| 8671 | 2P | BG14NUC-HC199 |
| 8672 | 2P | BG14NUC-HC200 |
| 8673 | RBD | BG14NUC-HC201 |
| 8674 | RBD | BG14NUC-HC202 |
| 8675 | 2P | BG14NUC-HC203 |
| 8676 | 2P | BG14NUC-HC204 |
| 8677 | 2P | BG14NUC-HC205 |
| 8678 | 2P | BG14NUC-HC206 |
| 8679 | 2P | BG14NUC-HC207 |
| 8680 | 2P | BG14NUC-HC208 |
| 8681 | 2P | BG14NUC-HC209 |
| 8682 | 2P | BG14NUC-HC210 |
| 8683 | 2P | BG14NUC-HC211 |
| 8684 | RBD | BG14NUC-HC212 |
| 8685 | 2P | BG14NUC-HC213 |
| 8686 | 2P | BG14NUC-HC214 |
| 8687 | RBD | BG14NUC-HC215 |
| 8688 | RBD | BG14NUC-HC216 |
| 8689 | 2P | BG14NUC-HC217 |
| 8690 | 2P | BG14NUC-HC218 |
| 8691 | 2P | BG14NUC-HC219 |
| 8692 | RBD | BG14NUC-HC220 |
| 8693 | 2P | BG14NUC-HC221 |
| 8694 | 2P | BG14NUC-HC222 |
| 8695 | 2P | BG14NUC-HC223 |
| 8696 | RBD | BG14NUC-HC224 |
| 8697 | 2P | BG14NUC-HC225 |
| 8698 | RBD | BG14NUC-HC226 |
| 8699 | 2P | BG14NUC-HC227 |
| 8700 | 2F | BG14NUC-HC228 |
| 8701 | RBD | BG14NUC-HC229 |
| 8702 | RBD | BG14NUC-HC230 |
| 8703 | 2P | BG14NUC-HC231 |
| 8704 | RBD | BG14NUC-HC232 |
| 8705 | 2P | BG14NUC-HC233 |
| 8706 | 2P | BG14NUC-HC234 |
| 8707 | 2P | BG14NUC-HC235 |
| 8708 | 2P | BG14NUC-HC236 |
| 8709 | 2P | BG14NUC-HC237 |
| 8710 | 2P | BG14NUC-HC238 |
| 8711 | 2P | BG14NUC-HC239 |
| 8712 | 2P | BG14NUC-HC240 |
| 8713 | 2P | BG14NUC-HC241 |
| 8714 | 2P | BG14NUC-HC242 |
| 8715 | 2P | BG14NUC-HC243 |
| 8716 | 2P | BG14NUC-HC244 |
| 8717 | 2P | BG14NUC-HC245 |
| 8718 | 2P | BG14NUC-HC246 |
| 8719 | 2P | BG14NUC-HC247 |
| 8720 | 2P | BG14NUC-HC248 |
| 8721 | 2P | BG14NUC-HC249 |
| 8722 | RBD | BG14NUC-HC250 |
| 8723 | 2P | BG14NUC-HC251 |
| 8724 | RBD | BG14NUC-HC252 |
| 8725 | RBD | BG14NUC-HC253 |
| 8726 | RBD | BG14NUC-HC254 |
| 8727 | RBD | BG14NUC-HC255 |
| 8728 | 2P | BG14NUC-HC256 |
| 8729 | RBD | BG14NUC-HC257 |
| 8730 | 2F | BG14NUC-HC258 |
| 8731 | 2P | BG14NUC-HC259 |
| 8732 | RBD | BG14NUC-HC260 |
| 8733 | RBD | BG14NUC-HC261 |
| 8734 | RBD | BG14NUC-HC262 |
| 8735 | 2P | BG14NUC-HC263 |
| 8736 | 2P | BG14NUC-HC264 |
| 8737 | 2P | BG14NUC-HC265 |
| 8738 | 2P | BG14NUC-HC266 |
| 8739 | RBD | BG14NUC-HC267 |
| 8740 | 2P | BG14NUC-HC268 |
| 8741 | RBD | BG14NUC-HC269 |
| 8742 | RBD | BG14NUC-HC270 |
| 8743 | 2P | BG14NUC-HC271 |
| 8744 | RBD | BG14NUC-HC272 |
| 8745 | RBD | BG14NUC-HC273 |
| 8746 | RBD | BG14NUC-HC274 |
| 8747 | RBD | BG14NUC-HC275 |
| 8748 | RBD | BG14NUC-HC276 |
| 8749 | 2P | BG14NUC-HC277 |
| 8750 | RBD | BG14NUC-HC278 |
| 8751 | 2P | BG14NUC-HC279 |
| 8752 | 2F | BG14NUC-HC280 |
| 8753 | RBD | BG14NUC-HC281 |
| 8754 | RBD | BG14NUC-HC282 |
| 8755 | 2P | BG14NUC-HC283 |
| 8756 | RBD | BG14NUC-HC284 |
| 8757 | RBD | BG14NUC-HC285 |
| 8758 | 2P | BG14NUC-HC286 |
| 8759 | RBD | BG14NUC-HC287 |
| 8760 | RBD | BG14NUC-HC288 |
| 8761 | 2P | BG14NUC-HC289 |
| 8762 | RBD | BG14NUC-HC290 |
| 8763 | 2P | BG14NUC-HC291 |
| 8764 | 2P | BG14NUC-HC292 |
| 8765 | 2P | BG14NUC-HC293 |
| 8766 | 2P | BG14NUC-HC294 |
| 8767 | 2P | BG14NUC-HC295 |
| 8768 | 2P | BG14NUC-HC296 |
| 8769 | 2P | BG14NUC-HC297 |
| 8770 | 2P | BG14NUC-HC298 |
| 8771 | RBD | BG14NUC-HC299 |
| 8772 | RBD | BG14NUC-HC300 |
| 8773 | RBD | BG14NUC-HC301 |
| 8774 | 2P | BG14NUC-HC302 |
| 8775 | RBD | BG14NUC-HC303 |
| 8776 | 2P | BG14NUC-HC304 |
| 8777 | RBD | BG14NUC-HC305 |
| 8778 | RBD | BG14NUC-HC306 |
| 8779 | 2P | BG14NUC-HC307 |
| 8780 | 2F | BG14NUC-HC308 |
| 8781 | 2P | BG14NUC-HC309 |
| 8782 | 2P | BG14NUC-HC310 |
| 8783 | 2P | BG14NUC-HC311 |
| 8784 | 2P | BG14NUC-HC312 |
| 8785 | RBD | BG14NUC-HC313 |
| 8786 | 2P | BG14NUC-HC314 |
| 8787 | 2P | BG14NUC-HC315 |
| 8788 | 2P | BG14NUC-HC316 |
| 8789 | RBD | BG14NUC-HC317 |
| 8790 | RBD | BG14NUC-HC318 |
| 8791 | RBD | BG14NUC-HC319 |
| 8792 | RBD | BG14NUC-HC320 |
| 8793 | 2P | BG14NUC-HC321 |
| 8794 | RBD | BG14NUC-HC322 |
| 8795 | RBD | BG14NUC-HC323 |
| 8796 | RBD | BG14NUC-HC324 |
| 8797 | 2P | BG14NUC-HC325 |
| 8798 | 2F | BG14NUC-HC326 |
| 8799 | 2P | BG14NUC-HC327 |
| 8800 | RBD | BG14NUC-HC328 |
| 8801 | RBD | BG14NUC-HC329 |
| 8802 | RBD | BG14NUC-HC330 |
| 8803 | 2P | BG14NUC-HC331 |
| 8804 | RBD | BG14NUC-HC332 |
| 8805 | RBD | BG14NUC-HC333 |
| 8806 | RBD | BG14NUC-HC334 |
| 8807 | 2F | BG14NUC-HC335 |
| 8808 | 2P | BG14NUC-HC336 |
| 8809 | 2P | BG14NUC-HC337 |
| 8810 | 2P | BG14NUC-HC338 |
| 8811 | RBD | BG14NUC-HC339 |
| 8812 | 2P | BG14NUC-HC340 |
| 8813 | RBD | BG14NUC-HC341 |
| 8814 | RBD | BG14NUC-HC342 |
| 8815 | 2F | BG14NUC-HC343 |
| 8816 | 2P | BG14NUC-HC344 |
| 8817 | RBD | BG14NUC-HC345 |
| 8818 | RBD | BG14NUC-HC346 |
| 8819 | RBD | BG14NUC-HC347 |
| 8820 | RBD | BG14NUC-HC348 |
| 8821 | 2P | BG14NUC-HC349 |
| 8822 | 2F | BG14NUC-HC350 |
| 8823 | 2P | BG14NUC-HC351 |
| 8824 | RBD | BG14NUC-HC352 |
| 8825 | RBD | BG14NUC-HC353 |
| 8826 | RBD | BG14NUC-HC354 |
| 8827 | 2F | BG14NUC-HC355 |
| 8828 | 2F | BG14NUC-HC356 |
| 8829 | 2F | BG14NUC-HC357 |
| 8830 | 2P | BG14NUC-HC358 |
| 8831 | 2P | BG14NUC-HC359 |
| 8832 | 2P | BG14NUC-HC360 |
| 8833 | 2P | BG14NUC-HC361 |
| 8834 | 2P | BG14NUC-HC362 |
| 8835 | RBD | BG14NUC-HC363 |
| 8836 | RBD | BG14NUC-HC364 |
| 8837 | 2F | BG14NUC-HC365 |
| 8838 | 2F | BG14NUC-HC366 |
| 8839 | 2P | BG14NUC-HC367 |
| 8840 | 2P | BG14NUC-HC368 |
| 8841 | 2P | BG14NUC-HC369 |
| 8842 | RBD | BG14NUC-HC370 |
| 8843 | 2P | BG14NUC-HC371 |
| 8844 | 2P | BG14NUC-HC372 |
| 8845 | RBD | BG14NUC-HC373 |
| 8846 | 2P | BG14NUC-HC374 |
| 8847 | 2P | BG14NUC-HC375 |
| 8848 | RBD | BG14NUC-HC376 |
| 8849 | RBD | BG14NUC-HC377 |
| 8850 | RBD | BG14NUC-HC378 |
| 8851 | 2P | BG14NUC-HC379 |
| 8852 | RBD | BG14NUC-HC380 |
| 8853 | 2P | BG14NUC-HC381 |
| 8854 | RBD | BG14NUC-HC382 |
| 8855 | RBD | BG14NUC-HC383 |
| 8856 | RBD | BG14NUC-HC384 |
| 8857 | RBD | BG14NUC-HC385 |
| 8858 | RBD | BG14NUC-HC386 |
| 8859 | 2P | BG14NUC-HC387 |
| 8860 | 2F | BG14NUC-HC388 |
| 8861 | 2P | BG14NUC-HC389 |
| 8862 | 2P | BG14NUC-HC390 |
| 8863 | RBD | BG14NUC-HC391 |
| 8864 | 2P | BG14NUC-HC392 |
| 8865 | 2P | BG14NUC-HC393 |
| 8866 | 2P | BG14NUC-HC394 |
| 8867 | 2P | BG14NUC-HC395 |
| 8868 | 2P | BG14NUC-HC396 |
| 8869 | 2P | BG14NUC-HC397 |
| 8870 | 2P | BG14NUC-HC398 |
| 8871 | 2P | BG14NUC-HC399 |
| 8872 | RBD | BG14NUC-HC400 |
| 8873 | 2P | BG14NUC-HC401 |
| 8874 | 2P | BG14NUC-HC402 |
| 8875 | 2P | BG14NUC-HC403 |
| 8876 | 2P | BG14NUC-HC404 |
| 8877 | 2P | BG14NUC-HC405 |
| 8878 | RBD | BG14NUC-HC406 |
| 8879 | 2F | BG14NUC-HC407 |
| 8880 | 2P | BG14NUC-HC408 |
| 8881 | RBD | BG14NUC-HC409 |
| 8882 | RBD | BG14NUC-HC410 |
| 8883 | 2P | BG14NUC-HC411 |
| 8884 | 2P | BG14NUC-HC412 |
| 8885 | 2P | BG14NUC-HC413 |
| 8886 | 2P | BG14NUC-HC414 |
| 8887 | 2P | BG14NUC-HC415 |
| 8888 | RBD | BG14NUC-HC416 |
| 8889 | 2P | BG14NUC-HC417 |
| 8890 | 2P | BG14NUC-HC418 |
| 8891 | RBD | BG14NUC-HC419 |
| 8892 | RBD | BG14NUC-HC420 |
| 8893 | 2P | BG14NUC-HC421 |
| 8894 | 2P | BG14NUC-HC422 |
| 8895 | 2P | BG14NUC-HC423 |
| 8896 | RBD | BG14NUC-HC424 |
| 8897 | 2P | BG14NUC-HC425 |
| 8898 | 2P | BG14NUC-HC426 |
| 8899 | RBD | BG14NUC-HC427 |
| 8900 | RBD | BG14NUC-HC428 |
| 8901 | 2F | BG14NUC-HC429 |
| 8902 | RBD | BG14NUC-HC430 |
| 8903 | 2P | BG14NUC-HC431 |
| 8904 | RBD | BG14NUC-HC432 |
| 8905 | 2P | BG14NUC-HC433 |
| 8906 | RBD | BG14NUC-HC434 |
| 8907 | RBD | BG14NUC-HC435 |
| 8908 | 2P | BG14NUC-HC436 |
| 8909 | RBD | BG14NUC-HC437 |
| 8910 | RBD | BG14NUC-HC438 |
| 8911 | RBD | BG14NUC-HC439 |
| 8912 | RBD | BG14NUC-HC440 |
| 8913 | 2P | BG14NUC-HC441 |
| 8914 | 2P | BG14NUC-HC442 |
| 8915 | RBD | BG14NUC-HC443 |
| 8916 | RBD | BG14NUC-HC444 |
| 8917 | 2P | BG14NUC-HC445 |
| 8918 | RBD | BG14NUC-HC446 |
| 8919 | 2P | BG14NUC-HC447 |
| 8920 | 2P | BG14NUC-HC448 |
| 8921 | 2P | BG14NUC-HC449 |
| 8922 | RBD | BG14NUC-HC450 |
| 8923 | RBD | BG14NUC-HC451 |
| 8924 | RBD | BG14NUC-HC452 |
| 8925 | RBD | BG14NUC-HC453 |
| 8926 | 2P | BG14NUC-HC454 |
| 8927 | RBD | BG14NUC-HC455 |
| 8928 | RBD | BG14NUC-HC456 |
| 8929 | RBD | BG14NUC-HC457 |
| 8930 | 2P | BG14NUC-HC458 |
| 8931 | 2P | BG14NUC-HC459 |
| 8932 | RBD | BG14NUC-HC460 |
| 8933 | 2P | BG14NUC-HC461 |
| 8934 | RBD | BG14NUC-HC462 |
| 8935 | 2P | BG14NUC-HC463 |
| 8936 | RBD | BG14NUC-HC464 |
| 8937 | RBD | BG14NUC-HC465 |
| 8938 | RBD | BG14NUC-HC466 |
| 8939 | RBD | BG14NUC-HC467 |
| 8940 | 2P | BG14NUC-HC468 |
| 8941 | 2P | BG14NUC-HC469 |
| 8942 | RBD | BG14NUC-HC470 |
| 8943 | 2P | BG14NUC-HC471 |
| 8944 | 2P | BG14NUC-HC472 |
| 8945 | RBD | BG14NUC-HC473 |
| 8946 | 2P | BG14NUC-HC474 |
| 8947 | RBD | BG14NUC-HC475 |
| 8948 | RBD | BG14NUC-HC476 |
| 8949 | 2P | BG14NUC-HC477 |
| 8950 | 2P | BG14NUC-HC478 |
| 8951 | RBD | BG14NUC-HC479 |
| 8952 | RBD | BG14NUC-HC480 |
| 8953 | RBD | BG14NUC-HC481 |
| 8954 | 2P | BG14NUC-HC482 |
| 8955 | 2P | BG14NUC-HC483 |
| 8956 | 2P | BG14NUC-HC484 |
| 8957 | 2P | BG14NUC-HC485 |
| 8958 | 2P | BG14NUC-HC486 |
| 8959 | 2P | BG14NUC-HC487 |
| 8960 | 2P | BG14NUC-HC488 |
| 8961 | RBD | BG14NUC-HC489 |
| 8962 | 2P | BG14NUC-HC490 |
| 8963 | RBD | BG14NUC-HC491 |
| 8964 | RBD | BG14NUC-HC492 |
| 8965 | 2P | BG14NUC-HC493 |
| 8966 | 2P | BG14NUC-HC494 |
| 8967 | 2P | BG14NUC-HC495 |
| 8968 | RBD | BG14NUC-HC496 |
| 8969 | RBD | BG14NUC-HC497 |
| 8970 | 2P | BG14NUC-HC498 |
| 8971 | RBD | BG14NUC-HC499 |
| 8972 | RBD | BG14NUC-HC500 |
| 8973 | RBD | BG14NUC-HC501 |
| 8974 | RBD | BG14NUC-HC502 |
| 8975 | RBD | BG14NUC-HC503 |
| 8976 | RBD | BG14NUC-HC504 |
| 8977 | 2P | BG14NUC-HC505 |
| 8978 | 2P | BG14NUC-HC506 |
| 8979 | 2P | BG14NUC-HC507 |
| 8980 | RBD | BG14NUC-HC508 |
| 8981 | RBD | BG14NUC-HC509 |
| 8982 | RBD | BG14NUC-HC510 |
| 8983 | RBD | BG14NUC-HC511 |
| 8984 | 2P | BG14NUC-HC512 |
| 8985 | RBD | BG14NUC-HC513 |
| 8986 | 2P | BG14NUC-HC514 |
| 8987 | 2P | BG14NUC-HC515 |
| 8988 | RBD | BG14NUC-HC516 |
| 8989 | 2P | BG14NUC-HC517 |
| 8990 | RBD | BG14NUC-HC518 |
| 8991 | 2P | BG14NUC-HC519 |
| 8992 | RBD | BG14NUC-HC520 |
| 8993 | RBD | BG14NUC-HC521 |
| 8994 | RBD | BG14NUC-HC522 |
| 8995 | 2P | BG14NUC-HC523 |
| 8996 | RBD | BG14NUC-HC524 |
| 8997 | 2P | BG14NUC-HC525 |
| 8998 | 2P | BG14NUC-HC526 |
| 8999 | 2P | BG14NUC-HC527 |
| 9000 | 2P | BG14NUC-HC528 |
| 9001 | 2P | BG14NUC-HC529 |
| 9002 | 2P | BG14NUC-HC530 |
| 9003 | RBD | BG14NUC-HC531 |
| 9004 | RBD | BG14NUC-HC532 |
| 9005 | RBD | BG14NUC-HC533 |
| 9006 | 2P | BG14NUC-HC534 |
| 9007 | 2P | BG14NUC-HC535 |
| 9008 | RBD | BG14NUC-LC1 |
| 9009 | RBD | BG14NUC-LC2 |
| 9010 | RBD | BG14NUC-LC3 |
| 9011 | RBD | BG14NUC-LC4 |
| 9012 | RBD | BG14NUC-LC5 |
| 9013 | RBD | BG14NUC-LC6 |
| 9014 | RBD | BG14NUC-LC7 |
| 9015 | RBD | BG14NUC-LC8 |
| 9016 | RBD | BG14NUC-LC9 |
| 9017 | RBD | BG14NUC-LC10 |
| 9018 | RBD | BG14NUC-LC11 |
| 9019 | RBD | BG14NUC-LC12 |
| 9020 | RBD | BG14NUC-LC13 |
| 9021 | RBD | BG14NUC-LC14 |
| 9022 | RBD | BG14NUC-LC15 |
| 9023 | RBD | BG14NUC-LC16 |
| 9024 | RBD | BG14NUC-LC17 |
| 9025 | RBD | BG14NUC-LC18 |
| 9026 | RBD | BG14NUC-LC19 |
| 9027 | RBD | BG14NUC-LC20 |
| 9028 | RBD | BG14NUC-LC21 |
| 9029 | RBD | BG14NUC-LC22 |
| 9030 | RBD | BG14NUC-LC23 |
| 9031 | RBD | BG14NUC-LC24 |
| 9032 | RBD | BG14NUC-LC25 |
| 9033 | 2P | BG14NUC-LC26 |
| 9034 | 2P | BG14NUC-LC27 |
| 9035 | 2P | BG14NUC-LC28 |
| 9036 | 2P | BG14NUC-LC29 |
| 9037 | 2P | BG14NUC-LC30 |
| 9038 | 2P | BG14NUC-LC31 |
| 9039 | 2P | BG14NUC-LC32 |
| 9040 | 2P | BG14NUC-LC33 |
| 9041 | 2P | BG14NUC-LC34 |
| 9042 | 2P | BG14NUC-LC35 |
| 9043 | 2P | BG14NUC-LC36 |
| 9044 | 2P | BG14NUC-LC37 |
| 9045 | 2P | BG14NUC-LC38 |
| 9046 | 2P | BG14NUC-LC39 |
| 9047 | 2P | BG14NUC-LC40 |
| 9048 | 2P | BG14NUC-LC41 |
| 9049 | 2P | BG14NUC-LC42 |
| 9050 | 2P | BG14NUC-LC43 |
| 9051 | 2P | BG14NUC-LC44 |
| 9052 | 2P | BG14NUC-LC45 |
| 9053 | 2P | BG14NUC-LC46 |
| 9054 | 2P | BG14NUC-LC47 |
| 9055 | 2P | BG14NUC-LC48 |
| 9056 | 2P | BG14NUC-LC49 |
| 9057 | RBD | BG14NUC-LC50 |
| 9058 | RBD | BG14NUC-LC51 |
| 9059 | RBD | BG14NUC-LC52 |
| 9060 | RBD | BG14NUC-LC53 |
| 9061 | RBD | BG14NUC-LC54 |
| 9062 | RBD | BG14NUC-LC55 |
| 9063 | RBD | BG14NUC-LC56 |
| 9064 | RBD | BG14NUC-LC57 |
| 9065 | RBD | BG14NUC-LC58 |
| 9066 | 2P | BG14NUC-LC59 |
| 9067 | 2P | BG14NUC-LC60 |
| 9068 | 2P | BG14NUC-LC61 |
| 9069 | 2P | BG14NUC-LC62 |
| 9070 | 2P | BG14NUC-LC63 |
| 9071 | 2P | BG14NUC-LC64 |
| 9072 | 2P | BG14NUC-LC65 |
| 9073 | RBD | BG14NUC-LC66 |
| 9074 | RBD | BG14NUC-LC67 |
| 9075 | RBD | BG14NUC-LC68 |
| 9076 | RBD | BG14NUC-LC69 |
| 9077 | RBD | BG14NUC-LC70 |
| 9078 | 2P | BG14NUC-LC71 |
| 9079 | 2P | BG14NUC-LC72 |
| 9080 | RBD | BG14NUC-LC73 |
| 9081 | RBD | BG14NUC-LC74 |
| 9082 | RBD | BG14NUC-LC75 |
| 9083 | 2P | BG14NUC-LC76 |
| 9084 | RBD | BG14NUC-LC77 |
| 9085 | RBD | BG14NUC-LC78 |
| 9086 | RBD | BG14NUC-LC79 |
| 9087 | RBD | BG14NUC-LC80 |
| 9088 | RBD | BG14NUC-LC81 |
| 9089 | RBD | BG14NUC-LC82 |
| 9090 | RBD | BG14NUC-LC83 |
| 9091 | RBD | BG14NUC-LC84 |
| 9092 | RBD | BG14NUC-LC85 |
| 9093 | 2P | BG14NUC-LC86 |
| 9094 | 2P | BG14NUC-LC87 |
| 9095 | 2P | BG14NUC-LC88 |
| 9096 | 2P | BG14NUC-LC89 |
| 9097 | RBD | BG14NUC-LC90 |
| 9098 | RBD | BG14NUC-LC91 |
| 9099 | RBD | BG14NUC-LC92 |
| 9100 | 2P | BG14NUC-LC93 |
| 9101 | 2P | BG14NUC-LC94 |
| 9102 | 2P | BG14NUC-LC95 |
| 9103 | RBD | BG14NUC-LC96 |
| 9104 | RBD | BG14NUC-LC97 |
| 9105 | RBD | BG14NUC-LC98 |
| 9106 | 2P | BG14NUC-LC99 |
| 9107 | RBD | BG14NUC-LC100 |
| 9108 | RBD | BG14NUC-LC101 |
| 9109 | RBD | BG14NUC-LC102 |
| 9110 | 2P | BG14NUC-LC103 |
| 9111 | RBD | BG14NUC-LC104 |
| 9112 | RBD | BG14NUC-LC105 |
| 9113 | RBD | BG14NUC-LC106 |
| 9114 | RBD | BG14NUC-LC107 |
| 9115 | RBD | BG14NUC-LC108 |
| 9116 | 2P | BG14NUC-LC109 |
| 9117 | 2P | BG14NUC-LC110 |
| 9118 | RBD | BG14NUC-LC111 |
| 9119 | RBD | BG14NUC-LC112 |
| 9120 | 2P | BG14NUC-LC113 |
| 9121 | 2P | BG14NUC-LC114 |
| 9122 | 2P | BG14NUC-LC115 |
| 9123 | RBD | BG14NUC-LC116 |
| 9124 | RBD | BG14NUC-LC117 |
| 9125 | RBD | BG14NUC-LC118 |
| 9126 | RBD | BG14NUC-LC119 |
| 9127 | RBD | BG14NUC-LC120 |
| 9128 | 2P | BG14NUC-LC121 |
| 9129 | 2P | BG14NUC-LC122 |
| 9130 | RBD | BG14NUC-LC123 |
| 9131 | RBD | BG14NUC-LC124 |
| 9132 | RBD | BG14NUC-LC125 |
| 9133 | 2P | BG14NUC-LC126 |
| 9134 | 2P | BG14NUC-LC127 |
| 9135 | RBD | BG14NUC-LC128 |
| 9136 | RBD | BG14NUC-LC129 |
| 9137 | RBD | BG14NUC-LC130 |
| 9138 | 2P | BG14NUC-LC131 |
| 9139 | 2P | BG14NUC-LC132 |
| 9140 | RBD | BG14NUC-LC133 |
| 9141 | RBD | BG14NUC-LC134 |
| 9142 | RBD | BG14NUC-LC135 |
| 9143 | RBD | BG14NUC-LC136 |
| 9144 | RBD | BG14NUC-LC137 |
| 9145 | RBD | BG14NUC-LC138 |
| 9146 | RBD | BG14NUC-LC139 |
| 9147 | RBD | BG14NUC-LC140 |
| 9148 | 2P | BG14NUC-LC141 |
| 9149 | 2P | BG14NUC-LC142 |
| 9150 | RBD | BG14NUC-LC143 |
| 9151 | 2P | BG14NUC-LC144 |
| 9152 | 2P | BG14NUC-LC145 |
| 9153 | 2P | BG14NUC-LC146 |
| 9154 | RBD | BG14NUC-LC147 |
| 9155 | RBD | BG14NUC-LC148 |
| 9156 | 2P | BG14NUC-LC149 |
| 9157 | 2P | BG14NUC-LC150 |
| 9158 | 2P | BG14NUC-LC151 |
| 9159 | 2P | BG14NUC-LC152 |
| 9160 | 2P | BG14NUC-LC153 |
| 9161 | RBD | BG14NUC-LC154 |
| 9162 | 2P | BG14NUC-LC155 |
| 9163 | 2P | BG14NUC-LC156 |
| 9164 | RBD | BG14NUC-LC157 |
| 9165 | 2P | BG14NUC-LC158 |
| 9166 | 2P | BG14NUC-LC159 |
| 9167 | 2P | BG14NUC-LC160 |
| 9168 | 2P | BG14NUC-LC161 |
| 9169 | 2P | BG14NUC-LC162 |
| 9170 | RBD | BG14NUC-LC163 |
| 9171 | RBD | BG14NUC-LC164 |
| 9172 | RBD | BG14NUC-LC165 |
| 9173 | 2P | BG14NUC-LC166 |
| 9174 | 2P | BG14NUC-LC167 |
| 9175 | 2P | BG14NUC-LC168 |
| 9176 | 2P | BG14NUC-LC169 |
| 9177 | RBD | BG14NUC-LC170 |
| 9178 | 2P | BG14NUC-LC171 |
| 9179 | RBD | BG14NUC-LC172 |
| 9180 | 2P | BG14NUC-LC173 |
| 9181 | 2P | BG14NUC-LC174 |
| 9182 | 2P | BG14NUC-LC175 |
| 9183 | RBD | BG14NUC-LC176 |
| 9184 | 2P | BG14NUC-LC177 |
| 9185 | RBD | BG14NUC-LC178 |
| 9186 | 2P | BG14NUC-LC179 |
| 9187 | 2P | BG14NUC-LC180 |
| 9188 | 2P | BG14NUC-LC181 |
| 9189 | 2P | BG14NUC-LC182 |
| 9190 | 2P | BG14NUC-LC183 |
| 9191 | RBD | BG14NUC-LC184 |
| 9192 | 2P | BG14NUC-LC185 |
| 9193 | 2P | BG14NUC-LC186 |
| 9194 | 2P | BG14NUC-LC187 |
| 9195 | RBD | BG14NUC-LC188 |
| 9196 | 2P | BG14NUC-LC189 |
| 9197 | 2P | BG14NUC-LC190 |
| 9198 | 2P | BG14NUC-LC191 |
| 9199 | RBD | BG14NUC-LC192 |
| 9200 | RBD | BG14NUC-LC193 |
| 9201 | RBD | BG14NUC-LC194 |
| 9202 | RBD | BG14NUC-LC195 |
| 9203 | RBD | BG14NUC-LC196 |
| 9204 | 2P | BG14NUC-LC197 |
| 9205 | 2P | BG14NUC-LC198 |
| 9206 | 2P | BG14NUC-LC199 |
| 9207 | 2P | BG14NUC-LC200 |
| 9208 | RBD | BG14NUC-LC201 |
| 9209 | RBD | BG14NUC-LC202 |
| 9210 | 2P | BG14NUC-LC203 |
| 9211 | 2P | BG14NUC-LC204 |
| 9212 | 2P | BG14NUC-LC205 |
| 9213 | 2P | BG14NUC-LC206 |
| 9214 | 2P | BG14NUC-LC207 |
| 9215 | 2P | BG14NUC-LC208 |
| 9216 | 2P | BG14NUC-LC209 |
| 9217 | 2P | BG14NUC-LC210 |
| 9218 | 2P | BG14NUC-LC211 |
| 9219 | RBD | BG14NUC-LC212 |
| 9220 | 2P | BG14NUC-LC213 |
| 9221 | 2P | BG14NUC-LC214 |
| 9222 | RBD | BG14NUC-LC215 |
| 9223 | RBD | BG14NUC-LC216 |
| 9224 | 2P | BG14NUC-LC217 |
| 9225 | 2P | BG14NUC-LC218 |
| 9226 | 2P | BG14NUC-LC219 |
| 9227 | RBD | BG14NUC-LC220 |
| 9228 | 2P | BG14NUC-LC221 |
| 9229 | 2P | BG14NUC-LC222 |
| 9230 | 2P | BG14NUC-LC223 |
| 9231 | RBD | BG14NUC-LC224 |
| 9232 | 2P | BG14NUC-LC225 |
| 9233 | RBD | BG14NUC-LC226 |
| 9234 | 2P | BG14NUC-LC227 |
| 9235 | 2P | BG14NUC-LC228 |
| 9236 | RBD | BG14NUC-LC229 |
| 9237 | RBD | BG14NUC-LC230 |
| 9238 | 2P | BG14NUC-LC231 |
| 9239 | RBD | BG14NUC-LC232 |
| 9240 | 2P | BG14NUC-LC233 |
| 9241 | 2P | BG14NUC-LC234 |
| 9242 | 2P | BG14NUC-LC235 |
| 9243 | 2P | BG14NUC-LC236 |
| 9244 | 2P | BG14NUC-LC237 |
| 9245 | 2P | BG14NUC-LC238 |
| 9246 | 2P | BG14NUC-LC239 |
| 9247 | 2P | BG14NUC-LC240 |
| 9248 | 2P | BG14NUC-LC241 |
| 9249 | 2P | BG14NUC-LC242 |
| 9250 | 2P | BG14NUC-LC243 |
| 9251 | 2P | BG14NUC-LC244 |
| 9252 | 2P | BG14NUC-LC245 |
| 9253 | 2P | BG14NUC-LC246 |
| 9254 | 2P | BG14NUC-LC247 |
| 9255 | 2P | BG14NUC-LC248 |
| 9256 | 2P | BG14NUC-LC249 |
| 9257 | RBD | BG14NUC-LC250 |
| 9258 | 2P | BG14NUC-LC251 |
| 9259 | RBD | BG14NUC-LC252 |
| 9260 | RBD | BG14NUC-LC253 |
| 9261 | RBD | BG14NUC-LC254 |
| 9262 | RBD | BG14NUC-LC255 |
| 9263 | 2P | BG14NUC-LC256 |
| 9264 | RBD | BG14NUC-LC257 |
| 9265 | 2P | BG14NUC-LC258 |
| 9266 | 2P | BG14NUC-LC259 |
| 9267 | RBD | BG14NUC-LC260 |
| 9268 | RBD | BG14NUC-LC261 |
| 9269 | RBD | BG14NUC-LC262 |
| 9270 | 2P | BG14NUC-LC263 |
| 9271 | 2P | BG14NUC-LC264 |
| 9272 | 2P | BG14NUC-LC265 |
| 9273 | 2P | BG14NUC-LC266 |
| 9274 | RBD | BG14NUC-LC267 |
| 9275 | 2P | BG14NUC-LC268 |
| 9276 | RBD | BG14NUC-LC269 |
| 9277 | RBD | BG14NUC-LC270 |
| 9278 | 2P | BG14NUC-LC271 |
| 9279 | RBD | BG14NUC-LC272 |
| 9280 | RBD | BG14NUC-LC273 |
| 9281 | RBD | BG14NUC-LC274 |
| 9282 | RBD | BG14NUC-LC275 |
| 9283 | RBD | BG14NUC-LC276 |
| 9284 | 2P | BG14NUC-LC277 |
| 9285 | RBD | BG14NUC-LC278 |
| 9286 | 2P | BG14NUC-LC279 |
| 9287 | 2P | BG14NUC-LC280 |
| 9288 | RBD | BG14NUC-LC281 |
| 9289 | RBD | BG14NUC-LC282 |
| 9290 | 2P | BG14NUC-LC283 |
| 9291 | RBD | BG14NUC-LC284 |
| 9292 | RBD | BG14NUC-LC285 |
| 9293 | 2P | BG14NUC-LC286 |
| 9294 | RBD | BG14NUC-LC287 |
| 9295 | RBD | BG14NUC-LC288 |
| 9296 | 2P | BG14NUC-LC289 |
| 9297 | RBD | BG14NUC-LC290 |
| 9298 | 2P | BG14NUC-LC291 |
| 9299 | 2P | BG14NUC-LC292 |
| 9300 | 2P | BG14NUC-LC293 |
| 9301 | 2P | BG14NUC-LC294 |
| 9302 | 2P | BG14NUC-LC295 |
| 9303 | 2P | BG14NUC-LC296 |
| 9304 | 2P | BG14NUC-LC297 |
| 9305 | 2P | BG14NUC-LC298 |
| 9306 | RBD | BG14NUC-LC299 |
| 9307 | RBD | BG14NUC-LC300 |
| 9308 | RBD | BG14NUC-LC301 |
| 9309 | 2P | BG14NUC-LC302 |
| 9310 | RBD | BG14NUC-LC303 |
| 9311 | 2P | BG14NUC-LC304 |
| 9312 | RBD | BG14NUC-LC305 |
| 9313 | RBD | BG14NUC-LC306 |
| 9314 | 2P | BG14NUC-LC307 |
| 9315 | 2P | BG14NUC-LC308 |
| 9316 | 2P | BG14NUC-LC309 |
| 9317 | 2P | BG14NUC-LC310 |
| 9318 | 2P | BG14NUC-LC311 |
| 9319 | 2P | BG14NUC-LC312 |
| 9320 | RBD | BG14NUC-LC313 |
| 9321 | 2P | BG14NUC-LC314 |
| 9322 | 2P | BG14NUC-LC315 |
| 9323 | 2P | BG14NUC-LC316 |
| 9324 | RBD | BG14NUC-LC317 |
| 9325 | RBD | BG14NUC-LC318 |
| 9326 | RBD | BG14NUC-LC319 |
| 9327 | RBD | BG14NUC-LC320 |
| 9328 | 2P | BG14NUC-LC321 |
| 9329 | RBD | BG14NUC-LC322 |
| 9330 | RBD | BG14NUC-LC323 |
| 9331 | RBD | BG14NUC-LC324 |
| 9332 | 2P | BG14NUC-LC325 |
| 9333 | 2P | BG14NUC-LC326 |
| 9334 | 2P | BG14NUC-LC327 |
| 9335 | RBD | BG14NUC-LC328 |
| 9336 | RBD | BG14NUC-LC329 |
| 9337 | RBD | BG14NUC-LC330 |
| 9338 | 2P | BG14NUC-LC331 |
| 9339 | RBD | BG14NUC-LC332 |
| 9340 | RBD | BG14NUC-LC333 |
| 9341 | RBD | BG14NUC-LC334 |
| 9342 | 2P | BG14NUC-LC335 |
| 9343 | 2P | BG14NUC-LC336 |
| 9344 | 2P | BG14NUC-LC337 |
| 9345 | 2P | BG14NUC-LC338 |
| 9346 | RBD | BG14NUC-LC339 |
| 9347 | 2P | BG14NUC-LC340 |
| 9348 | RBD | BG14NUC-LC341 |
| 9349 | RBD | BG14NUC-LC342 |
| 9350 | 2P | BG14NUC-LC343 |
| 9351 | 2P | BG14NUC-LC344 |
| 9352 | RBD | BG14NUC-LC345 |
| 9353 | RBD | BG14NUC-LC346 |
| 9354 | RBD | BG14NUC-LC347 |
| 9355 | RBD | BG14NUC-LC348 |
| 9356 | 2P | BG14NUC-LC349 |
| 9357 | 2P | BG14NUC-LC350 |
| 9358 | 2P | BG14NUC-LC351 |
| 9359 | RBD | BG14NUC-LC352 |
| 9360 | RBD | BG14NUC-LC353 |
| 9361 | RBD | BG14NUC-LC354 |
| 9362 | 2P | BG14NUC-LC355 |
| 9363 | 2P | BG14NUC-LC356 |
| 9364 | 2P | BG14NUC-LC357 |
| 9365 | 2P | BG14NUC-LC358 |
| 9366 | 2P | BG14NUC-LC359 |
| 9367 | 2P | BG14NUC-LC360 |
| 9368 | 2P | BG14NUC-LC361 |
| 9369 | 2P | BG14NUC-LC362 |
| 9370 | RBD | BG14NUC-LC363 |
| 9371 | RBD | BG14NUC-LC364 |
| 9372 | 2P | BG14NUC-LC365 |
| 9373 | 2P | BG14NUC-LC366 |
| 9374 | 2P | BG14NUC-LC367 |
| 9375 | 2P | BG14NUC-LC368 |
| 9376 | 2P | BG14NUC-LC369 |
| 9377 | RBD | BG14NUC-LC370 |
| 9378 | 2P | BG14NUC-LC371 |
| 9379 | 2P | BG14NUC-LC372 |
| 9380 | RBD | BG14NUC-LC373 |
| 9381 | 2P | BG14NUC-LC374 |
| 9382 | 2P | BG14NUC-LC375 |
| 9383 | RBD | BG14NUC-LC376 |
| 9384 | RBD | BG14NUC-LC377 |
| 9385 | RBD | BG14NUC-LC378 |
| 9386 | 2P | BG14NUC-LC379 |
| 9387 | RBD | BG14NUC-LC380 |
| 9388 | 2P | BG14NUC-LC381 |
| 9389 | RBD | BG14NUC-LC382 |
| 9390 | RBD | BG14NUC-LC383 |
| 9391 | RBD | BG14NUC-LC384 |
| 9392 | RBD | BG14NUC-LC385 |
| 9393 | RBD | BG14NUC-LC386 |
| 9394 | 2P | BG14NUC-LC387 |
| 9395 | 2P | BG14NUC-LC388 |
| 9396 | 2P | BG14NUC-LC389 |
| 9397 | 2P | BG14NUC-LC390 |
| 9398 | RBD | BG14NUC-LC391 |
| 9399 | 2P | BG14NUC-LC392 |
| 9400 | 2P | BG14NUC-LC393 |
| 9401 | 2P | BG14NUC-LC394 |
| 9402 | 2P | BG14NUC-LC395 |
| 9403 | 2P | BG14NUC-LC396 |
| 9404 | 2P | BG14NUC-LC397 |
| 9405 | 2P | BG14NUC-LC398 |
| 9406 | 2P | BG14NUC-LC399 |
| 9407 | RBD | BG14NUC-LC400 |
| 9408 | 2P | BG14NUC-LC401 |
| 9409 | 2P | BG14NUC-LC402 |
| 9410 | 2P | BG14NUC-LC403 |
| 9411 | 2P | BG14NUC-LC404 |
| 9412 | 2P | BG14NUC-LC405 |
| 9413 | RBD | BG14NUC-LC406 |
| 9414 | 2P | BG14NUC-LC407 |
| 9415 | 2P | BG14NUC-LC408 |
| 9416 | RBD | BG14NUC-LC409 |
| 9417 | RBD | BG14NUC-LC410 |
| 9418 | 2P | BG14NUC-LC411 |
| 9419 | 2P | BG14NUC-LC412 |
| 9420 | 2P | BG14NUC-LC413 |
| 9421 | 2P | BG14NUC-LC414 |
| 9422 | 2P | BG14NUC-LC415 |
| 9423 | RBD | BG14NUC-LC416 |
| 9424 | 2P | BG14NUC-LC417 |
| 9425 | 2P | BG14NUC-LC418 |
| 9426 | RBD | BG14NUC-LC419 |
| 9427 | RBD | BG14NUC-LC420 |
| 9428 | 2P | BG14NUC-LC421 |
| 9429 | 2P | BG14NUC-LC422 |
| 9430 | 2P | BG14NUC-LC423 |
| 9431 | RBD | BG14NUC-LC424 |
| 9432 | 2P | BG14NUC-LC425 |
| 9433 | 2P | BG14NUC-LC426 |
| 9434 | RBD | BG14NUC-LC427 |
| 9435 | RBD | BG14NUC-LC428 |
| 9436 | 2P | BG14NUC-LC429 |
| 9437 | RBD | BG14NUC-LC430 |
| 9438 | 2P | BG14NUC-LC431 |
| 9439 | RBD | BG14NUC-LC432 |
| 9440 | 2P | BG14NUC-LC433 |
| 9441 | RBD | BG14NUC-LC434 |
| 9442 | RBD | BG14NUC-LC435 |
| 9443 | 2P | BG14NUC-LC436 |
| 9444 | RBD | BG14NUC-LC437 |
| 9445 | RBD | BG14NUC-LC438 |
| 9446 | RBD | BG14NUC-LC439 |
| 9447 | RBD | BG14NUC-LC440 |
| 9448 | 2P | BG14NUC-LC441 |
| 9449 | 2P | BG14NUC-LC442 |
| 9450 | RBD | BG14NUC-LC443 |
| 9451 | RBD | BG14NUC-LC444 |
| 9452 | 2P | BG14NUC-LC445 |
| 9453 | RBD | BG14NUC-LC446 |
| 9454 | 2P | BG14NUC-LC447 |
| 9455 | 2P | BG14NUC-LC448 |
| 9456 | 2P | BG14NUC-LC449 |
| 9457 | RBD | BG14NUC-LC450 |
| 9458 | RBD | BG14NUC-LC451 |
| 9459 | RBD | BG14NUC-LC452 |
| 9460 | RBD | BG14NUC-LC453 |
| 9461 | 2P | BG14NUC-LC454 |
| 9462 | RBD | BG14NUC-LC455 |
| 9463 | RBD | BG14NUC-LC456 |
| 9464 | RBD | BG14NUC-LC457 |
| 9465 | 2P | BG14NUC-LC458 |
| 9466 | 2P | BG14NUC-LC459 |
| 9467 | RBD | BG14NUC-LC460 |
| 9468 | 2P | BG14NUC-LC461 |
| 9469 | RBD | BG14NUC-LC462 |
| 9470 | 2P | BG14NUC-LC463 |
| 9471 | RBD | BG14NUC-LC464 |
| 9472 | RBD | BG14NUC-LC465 |
| 9473 | RBD | BG14NUC-LC466 |
| 9474 | RBD | BG14NUC-LC467 |
| 9475 | 2P | BG14NUC-LC468 |
| 9476 | 2P | BG14NUC-LC469 |
| 9477 | RBD | BG14NUC-LC470 |
| 9478 | 2P | BG14NUC-LC471 |
| 9479 | 2P | BG14NUC-LC472 |
| 9480 | RBD | BG14NUC-LC473 |
| 9481 | 2P | BG14NUC-LC474 |
| 9482 | RBD | BG14NUC-LC475 |
| 9483 | RBD | BG14NUC-LC476 |
| 9484 | 2P | BG14NUC-LC477 |
| 9485 | 2P | BG14NUC-LC478 |
| 9486 | RBD | BG14NUC-LC479 |
| 9487 | RBD | BG14NUC-LC480 |
| 9488 | RBD | BG14NUC-LC481 |
| 9489 | 2P | BG14NUC-LC482 |
| 9490 | 2P | BG14NUC-LC483 |
| 9491 | 2P | BG14NUC-LC484 |
| 9492 | 2P | BG14NUC-LC485 |
| 9493 | 2P | BG14NUC-LC486 |
| 9494 | 2P | BG14NUC-LC487 |
| 9495 | 2P | BG14NUC-LC488 |
| 9496 | RBD | BG14NUC-LC489 |
| 9497 | 2P | BG14NUC-LC490 |
| 9498 | RBD | BG14NUC-LC491 |
| 9499 | RBD | BG14NUC-LC492 |
| 9500 | 2P | BG14NUC-LC493 |
| 9501 | 2P | BG14NUC-LC494 |
| 9502 | 2P | BG14NUC-LC495 |
| 9503 | RBD | BG14NUC-LC496 |
| 9504 | RBD | BG14NUC-LC497 |
| 9505 | 2P | BG14NUC-LC498 |
| 9506 | RBD | BG14NUC-LC499 |
| 9507 | RBD | BG14NUC-LC500 |
| 9508 | RBD | BG14NUC-LC501 |
| 9509 | RBD | BG14NUC-LC502 |
| 9510 | RBD | BG14NUC-LC503 |
| 9511 | RBD | BG14NUC-LC504 |
| 9512 | 2P | BG14NUC-LC505 |
| 9513 | 2P | BG14NUC-LC506 |
| 9514 | 2P | BG14NUC-LC507 |
| 9515 | RBD | BG14NUC-LC508 |
| 9516 | RBD | BG14NUC-LC509 |
| 9517 | RBD | BG14NUC-LC510 |
| 9518 | RBD | BG14NUC-LC511 |
| 9519 | 2P | BG14NUC-LC512 |
| 9520 | RBD | BG14NUC-LC513 |
| 9521 | 2P | BG14NUC-LC514 |
| 9522 | 2P | BG14NUC-LC515 |
| 9523 | RBD | BG14NUC-LC516 |
| 9524 | 2P | BG14NUC-LC517 |
| 9525 | RBD | BG14NUC-LC518 |
| 9526 | 2P | BG14NUC-LC519 |
| 9527 | RBD | BG14NUC-LC520 |
| 9528 | RBD | BG14NUC-LC521 |
| 9529 | RBD | BG14NUC-LC522 |
| 9530 | 2P | BG14NUC-LC523 |
| 9531 | RBD | BG14NUC-LC524 |
| 9532 | 2P | BG14NUC-LC525 |
| 9533 | 2P | BG14NUC-LC526 |
| 9534 | 2P | BG14NUC-LC527 |
| 9535 | 2P | BG14NUC-LC528 |
| 9536 | 2P | BG14NUC-LC529 |
| 9537 | 2P | BG14NUC-LC530 |
| 9538 | RBD | BG14NUC-LC531 |
| 9539 | RBD | BG14NUC-LC532 |
| 9540 | RBD | BG14NUC-LC533 |
| 9541 | 2P | BG14NUC-LC534 |
| 9542 | 2P | BG14NUC-LC535 |
| 9543 | 2P | BG18NUC-HC1 |
| 9544 | 2P | BG18NUC-HC2 |
| 9545 | RBD | BG18NUC-HC3 |
| 9546 | 2P | BG18NUC-HC4 |
| 9547 | 2P | BG18NUC-HC5 |
| 9548 | RBD | BG18NUC-HC6 |
| 9549 | 2P | BG18NUC-HC7 |
| 9550 | 2P | BG18NUC-HC8 |
| 9551 | RBD | BG18NUC-HC9 |
| 9552 | 2P | BG18NUC-HC10 |
| 9553 | 2P | BG18NUC-HC11 |
| 9554 | 2P | BG18NUC-HC12 |
| 9555 | 2P | BG18NUC-HC13 |
| 9556 | 2P | BG18NUC-HC14 |
| 9557 | 2P | BG18NUC-HC15 |
| 9558 | 2P | BG18NUC-HC16 |
| 9559 | RBD | BG18NUC-HC17 |
| 9560 | 2P | BG18NUC-HC18 |
| 9561 | 2P | BG18NUC-HC19 |
| 9562 | RBD | BG18NUC-HC20 |
| 9563 | 2P | BG18NUC-HC21 |
| 9564 | RBD | BG18NUC-HC22 |
| 9565 | 2P | BG18NUC-HC23 |
| 9566 | 2P | BG18NUC-HC24 |
| 9567 | 2P | BG18NUC-HC25 |
| 9568 | 2P | BG18NUC-HC26 |
| 9569 | 2P | BG18NUC-HC27 |
| 9570 | 2P | BG18NUC-HC28 |
| 9571 | 2P | BG18NUC-HC29 |
| 9572 | 2P | BG18NUC-HC30 |
| 9573 | RBD | BG18NUC-HC31 |
| 9574 | RBD | BG18NUC-HC32 |
| 9575 | RBD | BG18NUC-HC33 |
| 9576 | 2P | BG18NUC-HC34 |
| 9577 | 2P | BG18NUC-HC35 |
| 9578 | 2F | BG18NUC-HC36 |
| 9579 | 2P | BG18NUC-HC37 |
| 9580 | 2P | BG18NUC-HC38 |
| 9581 | 2P | BG18NUC-HC39 |
| 9582 | 2P | BG18NUC-HC40 |
| 9583 | RBD | BG18NUC-HC41 |
| 9584 | RBD | BG18NUC-HC42 |
| 9585 | 2P | BG18NUC-HC43 |
| 9586 | 2P | BG18NUC-HC44 |
| 9587 | 2P | BG18NUC-HC45 |
| 9588 | RBD | BG18NUC-HC46 |
| 9589 | 2P | BG18NUC-HC47 |
| 9590 | 2P | BG18NUC-HC48 |
| 9591 | 2P | BG18NUC-HC49 |
| 9592 | 2P | BG18NUC-HC50 |
| 9593 | 2P | BG18NUC-HC51 |
| 9594 | 2P | BG18NUC-HC52 |
| 9595 | 2P | BG18NUC-HC53 |
| 9596 | RBD | BG18NUC-HC54 |
| 9597 | 2P | BG18NUC-HC55 |
| 9598 | 2P | BG18NUC-HC56 |
| 9599 | 2P | BG18NUC-HC57 |
| 9600 | 2P | BG18NUC-HC58 |
| 9601 | RBD | BG18NUC-HC59 |
| 9602 | 2P | BG18NUC-HC60 |
| 9603 | 2P | BG18NUC-HC61 |
| 9604 | 2P | BG18NUC-HC62 |
| 9605 | RBD | BG18NUC-HC63 |
| 9606 | RBD | BG18NUC-HC64 |
| 9607 | 2P | BG18NUC-HC65 |
| 9608 | 2P | BG18NUC-HC66 |
| 9609 | 2P | BG18NUC-HC67 |
| 9610 | 2P | BG18NUC-HC68 |
| 9611 | RBD | BG18NUC-HC69 |
| 9612 | 2P | BG18NUC-HC70 |
| 9613 | RBD | BG18NUC-HC71 |
| 9614 | 2P | BG18NUC-HC72 |
| 9615 | 2P | BG18NUC-HC73 |
| 9616 | 2P | BG18NUC-HC74 |
| 9617 | 2P | BG18NUC-HC75 |
| 9618 | 2P | BG18NUC-HC76 |
| 9619 | RBD | BG18NUC-HC77 |
| 9620 | RBD | BG18NUC-HC78 |
| 9621 | 2P | BG18NUC-HC79 |
| 9622 | 2P | BG18NUC-HC80 |
| 9623 | 2P | BG18NUC-HC81 |
| 9624 | 2P | BG18NUC-HC82 |
| 9625 | 2P | BG18NUC-HC83 |
| 9626 | 2P | BG18NUC-HC84 |
| 9627 | 2P | BG18NUC-HC85 |
| 9628 | 2P | BG18NUC-HC86 |
| 9629 | 2P | BG18NUC-HC87 |
| 9630 | 2P | BG18NUC-HC88 |
| 9631 | 2P | BG18NUC-HC89 |
| 9632 | 2P | BG18NUC-HC90 |
| 9633 | RBD | BG18NUC-HC91 |
| 9634 | RBD | BG18NUC-HC92 |
| 9635 | 2P | BG18NUC-HC93 |
| 9636 | 2P | BG18NUC-HC94 |
| 9637 | 2P | BG18NUC-HC95 |
| 9638 | RBD | BG18NUC-HC96 |
| 9639 | 2P | BG18NUC-HC97 |
| 9640 | 2P | BG18NUC-HC98 |
| 9641 | 2P | BG18NUC-HC99 |
| 9642 | 2P | BG18NUC-HC100 |
| 9643 | RBD | BG18NUC-HC101 |
| 9644 | 2P | BG18NUC-HC102 |
| 9645 | RBD | BG18NUC-HC103 |
| 9646 | RBD | BG18NUC-HC104 |
| 9647 | 2P | BG18NUC-HC105 |
| 9648 | RBD | BG18NUC-HC106 |
| 9649 | RBD | BG18NUC-HC107 |
| 9650 | 2P | BG18NUC-HC108 |
| 9651 | 2P | BG18NUC-HC109 |
| 9652 | RBD | BG18NUC-HC110 |
| 9653 | 2P | BG18NUC-HC111 |
| 9654 | 2P | BG18NUC-HC112 |
| 9655 | 2P | BG18NUC-HC113 |
| 9656 | 2P | BG18NUC-HC114 |
| 9657 | 2P | BG18NUC-HC115 |
| 9658 | RBD | BG18NUC-HC116 |
| 9659 | 2P | BG18NUC-HC117 |
| 9660 | 2P | BG18NUC-HC118 |
| 9661 | 2P | BG18NUC-HC119 |
| 9662 | 2P | BG18NUC-HC120 |
| 9663 | 2P | BG18NUC-HC121 |
| 9664 | 2P | BG18NUC-HC122 |
| 9665 | 2P | BG18NUC-HC123 |
| 9666 | 2P | BG18NUC-HC124 |
| 9667 | 2P | BG18NUC-HC125 |
| 9668 | 2P | BG18NUC-HC126 |
| 9669 | 2P | BG18NUC-HC127 |
| 9670 | 2P | BG18NUC-HC128 |
| 9671 | 2P | BG18NUC-HC129 |
| 9672 | 2P | BG18NUC-HC130 |
| 9673 | 2P | BG18NUC-HC131 |
| 9674 | RBD | BG18NUC-HC132 |
| 9675 | 2P | BG18NUC-HC133 |
| 9676 | RBD | BG18NUC-HC134 |
| 9677 | 2P | BG18NUC-HC135 |
| 9678 | 2P | BG18NUC-HC136 |
| 9679 | RBD | BG18NUC-HC137 |
| 9680 | 2P | BG18NUC-HC138 |
| 9681 | RBD | BG18NUC-HC139 |
| 9682 | RBD | BG18NUC-HC140 |
| 9683 | RBD | BG18NUC-HC141 |
| 9684 | 2P | BG18NUC-HC142 |
| 9685 | 2P | BG18NUC-HC143 |
| 9686 | 2P | BG18NUC-HC144 |
| 9687 | 2P | BG18NUC-HC145 |
| 9688 | 2P | BG18NUC-HC146 |
| 9689 | 2P | BG18NUC-HC147 |
| 9690 | 2P | BG18NUC-HC148 |
| 9691 | 2P | BG18NUC-HC149 |
| 9692 | 2P | BG18NUC-HC150 |
| 9693 | 2P | BG18NUC-HC151 |
| 9694 | 2P | BG18NUC-HC152 |
| 9695 | 2P | BG18NUC-HC153 |
| 9696 | 2P | BG18NUC-HC154 |
| 9697 | RBD | BG18NUC-HC155 |
| 9698 | 2P | BG18NUC-HC156 |
| 9699 | 2P | BG18NUC-HC157 |
| 9700 | 2P | BG18NUC-HC158 |
| 9701 | 2P | BG18NUC-HC159 |
| 9702 | 2P | BG18NUC-HC160 |
| 9703 | 2P | BG18NUC-HC161 |
| 9704 | 2P | BG18NUC-HC162 |
| 9705 | RBD | BG18NUC-HC163 |
| 9706 | 2P | BG18NUC-HC164 |
| 9707 | RBD | BG18NUC-HC165 |
| 9708 | RBD | BG18NUC-HC166 |
| 9709 | 2P | BG18NUC-HC167 |
| 9710 | 2P | BG18NUC-HC168 |
| 9711 | RBD | BG18NUC-HC169 |
| 9712 | 2P | BG18NUC-HC170 |
| 9713 | 2P | BG18NUC-HC171 |
| 9714 | 2P | BG18NUC-HC172 |
| 9715 | 2P | BG18NUC-HC173 |
| 9716 | 2P | BG18NUC-HC174 |
| 9717 | 2P | BG18NUC-HC175 |
| 9718 | 2P | BG18NUC-HC176 |
| 9719 | 2P | BG18NUC-HC177 |
| 9720 | 2P | BG18NUC-HC178 |
| 9721 | 2P | BG18NUC-HC179 |
| 9722 | RBD | BG18NUC-HC180 |
| 9723 | RBD | BG18NUC-HC181 |
| 9724 | 2P | BG18NUC-HC182 |
| 9725 | 2P | BG18NUC-HC183 |
| 9726 | 2P | BG18NUC-HC184 |
| 9727 | 2P | BG18NUC-HC185 |
| 9728 | RBD | BG18NUC-HC186 |
| 9729 | 2P | BG18NUC-HC187 |
| 9730 | 2P | BG18NUC-HC188 |
| 9731 | 2P | BG18NUC-HC189 |
| 9732 | 2P | BG18NUC-HC190 |
| 9733 | 2P | BG18NUC-HC191 |
| 9734 | 2P | BG18NUC-HC192 |
| 9735 | RBD | BG18NUC-HC193 |
| 9736 | 2P | BG18NUC-HC194 |
| 9737 | 2P | BG18NUC-HC195 |
| 9738 | RBD | BG18NUC-HC196 |
| 9739 | RBD | BG18NUC-HC197 |
| 9740 | RBD | BG18NUC-HC198 |
| 9741 | RBD | BG18NUC-HC199 |
| 9742 | 2P | BG18NUC-HC200 |
| 9743 | RBD | BG18NUC-HC201 |
| 9744 | 2P | BG18NUC-HC202 |
| 9745 | RBD | BG18NUC-HC203 |
| 9746 | 2P | BG18NUC-HC204 |
| 9747 | RBD | BG18NUC-HC205 |
| 9748 | 2P | BG18NUC-HC206 |
| 9749 | 2P | BG18NUC-HC207 |
| 9750 | 2P | BG18NUC-HC208 |
| 9751 | 2P | BG18NUC-HC209 |
| 9752 | 2P | BG18NUC-HC210 |
| 9753 | 2P | BG18NUC-HC211 |
| 9754 | RBD | BG18NUC-HC212 |
| 9755 | 2P | BG18NUC-HC213 |
| 9756 | RBD | BG18NUC-HC214 |
| 9757 | RBD | BG18NUC-HC215 |
| 9758 | RBD | BG18NUC-HC216 |
| 9759 | RBD | BG18NUC-HC217 |
| 9760 | 2P | BG18NUC-HC218 |
| 9761 | RBD | BG18NUC-HC219 |
| 9762 | 2P | BG18NUC-HC220 |
| 9763 | 2P | BG18NUC-HC221 |
| 9764 | 2P | BG18NUC-HC222 |
| 9765 | 2P | BG18NUC-HC223 |
| 9766 | RBD | BG18NUC-HC224 |
| 9767 | RBD | BG18NUC-HC225 |
| 9768 | 2P | BG18NUC-HC226 |
| 9769 | 2P | BG18NUC-HC227 |
| 9770 | 2P | BG18NUC-HC228 |
| 9771 | 2P | BG18NUC-HC229 |
| 9772 | RBD | BG18NUC-HC230 |
| 9773 | 2P | BG18NUC-HC231 |
| 9774 | RBD | BG18NUC-HC232 |
| 9775 | 2P | BG18NUC-HC233 |
| 9776 | 2P | BG18NUC-HC234 |
| 9777 | 2P | BG18NUC-HC235 |
| 9778 | 2P | BG18NUC-HC236 |
| 9779 | 2P | BG18NUC-HC237 |
| 9780 | RBD | BG18NUC-HC238 |
| 9781 | RBD | BG18NUC-HC239 |
| 9782 | RBD | BG18NUC-HC240 |
| 9783 | 2P | BG18NUC-HC241 |
| 9784 | RBD | BG18NUC-HC242 |
| 9785 | 2P | BG18NUC-HC243 |
| 9786 | 2P | BG18NUC-HC244 |
| 9787 | 2P | BG18NUC-HC245 |
| 9788 | 2P | BG18NUC-HC246 |
| 9789 | 2P | BG18NUC-HC247 |
| 9790 | 2P | BG18NUC-HC248 |
| 9791 | 2P | BG18NUC-HC249 |
| 9792 | 2P | BG18NUC-HC250 |
| 9793 | 2P | BG18NUC-HC251 |
| 9794 | RBD | BG18NUC-HC252 |
| 9795 | 2P | BG18NUC-HC253 |
| 9796 | RBD | BG18NUC-HC254 |
| 9797 | RBD | BG18NUC-HC255 |
| 9798 | 2P | BG18NUC-HC256 |
| 9799 | 2P | BG18NUC-HC257 |
| 9800 | 2P | BG18NUC-HC258 |
| 9801 | RBD | BG18NUC-HC259 |
| 9802 | 2P | BG18NUC-HC260 |
| 9803 | RBD | BG18NUC-HC261 |
| 9804 | 2P | BG18NUC-HC262 |
| 9805 | RBD | BG18NUC-HC263 |
| 9806 | 2P | BG18NUC-HC264 |
| 9807 | 2P | BG18NUC-HC265 |
| 9808 | 2P | BG18NUC-HC266 |
| 9809 | RBD | BG18NUC-HC267 |
| 9810 | 2P | BG18NUC-HC268 |
| 9811 | 2P | BG18NUC-HC269 |
| 9812 | 2P | BG18NUC-HC270 |
| 9813 | 2P | BG18NUC-HC271 |
| 9814 | 2P | BG18NUC-HC272 |
| 9815 | 2P | BG18NUC-HC273 |
| 9816 | 2P | BG18NUC-HC274 |
| 9817 | 2P | BG18NUC-HC275 |
| 9818 | 2P | BG18NUC-HC276 |
| 9819 | 2P | BG18NUC-HC277 |
| 9820 | 2P | BG18NUC-HC278 |
| 9821 | RBD | BG18NUC-HC279 |
| 9822 | 2P | BG18NUC-HC280 |
| 9823 | 2P | BG18NUC-HC281 |
| 9824 | RBD | BG18NUC-HC282 |
| 9825 | 2P | BG18NUC-HC283 |
| 9826 | RBD | BG18NUC-HC284 |
| 9827 | 2P | BG18NUC-HC285 |
| 9828 | RBD | BG18NUC-HC286 |
| 9829 | RBD | BG18NUC-HC287 |
| 9830 | 2P | BG18NUC-HC288 |
| 9831 | 2P | BG18NUC-HC289 |
| 9832 | 2P | BG18NUC-HC290 |
| 9833 | RBD | BG18NUC-HC291 |
| 9834 | RBD | BG18NUC-HC292 |
| 9835 | RBD | BG18NUC-HC293 |
| 9836 | 2P | BG18NUC-HC294 |
| 9837 | 2P | BG18NUC-HC295 |
| 9838 | 2P | BG18NUC-HC296 |
| 9839 | 2P | BG18NUC-HC297 |
| 9840 | 2P | BG18NUC-HC298 |
| 9841 | RBD | BG18NUC-HC299 |
| 9842 | 2P | BG18NUC-HC300 |
| 9843 | 2P | BG18NUC-HC301 |
| 9844 | 2P | BG18NUC-HC302 |
| 9845 | 2P | BG18NUC-HC303 |
| 9846 | 2P | BG18NUC-HC304 |
| 9847 | 2P | BG18NUC-HC305 |
| 9848 | 2P | BG18NUC-HC306 |
| 9849 | 2P | BG18NUC-HC307 |
| 9850 | 2P | BG18NUC-HC308 |
| 9851 | RBD | BG18NUC-HC309 |
| 9852 | 2P | BG18NUC-HC310 |
| 9853 | RBD | BG18NUC-HC311 |
| 9854 | 2P | BG18NUC-HC312 |
| 9855 | 2P | BG18NUC-HC313 |
| 9856 | 2P | BG18NUC-HC314 |
| 9857 | RBD | BG18NUC-HC315 |
| 9858 | 2F | BG18NUC-HC316 |
| 9859 | 2P | BG18NUC-HC317 |
| 9860 | 2P | BG18NUC-HC318 |
| 9861 | 2P | BG18NUC-HC319 |
| 9862 | 2P | BG18NUC-HC320 |
| 9863 | 2P | BG18NUC-HC321 |
| 9864 | RBD | BG18NUC-HC322 |
| 9865 | RBD | BG18NUC-HC323 |
| 9866 | RBD | BG18NUC-HC324 |
| 9867 | RBD | BG18NUC-HC325 |
| 9868 | 2P | BG18NUC-HC326 |
| 9869 | 2P | BG18NUC-HC327 |
| 9870 | RBD | BG18NUC-HC328 |
| 9871 | 2P | BG18NUC-HC329 |
| 9872 | 2P | BG18NUC-HC330 |
| 9873 | 2P | BG18NUC-HC331 |
| 9874 | 2P | BG18NUC-HC332 |
| 9875 | 2P | BG18NUC-HC333 |
| 9876 | RBD | BG18NUC-HC334 |
| 9877 | 2P | BG18NUC-HC335 |
| 9878 | RBD | BG18NUC-HC336 |
| 9879 | RBD | BG18NUC-HC337 |
| 9880 | 2P | BG18NUC-HC338 |
| 9881 | 2P | BG18NUC-HC339 |
| 9882 | 2P | BG18NUC-HC340 |
| 9883 | RBD | BG18NUC-HC341 |
| 9884 | 2P | BG18NUC-HC342 |
| 9885 | RBD | BG18NUC-HC343 |
| 9886 | 2P | BG18NUC-HC344 |
| 9887 | 2P | BG18NUC-HC345 |
| 9888 | 2P | BG18NUC-HC346 |
| 9889 | 2P | BG18NUC-HC347 |
| 9890 | 2P | BG18NUC-HC348 |
| 9891 | 2P | BG18NUC-HC349 |
| 9892 | 2P | BG18NUC-HC350 |
| 9893 | RBD | BG18NUC-HC351 |
| 9894 | 2P | BG18NUC-HC352 |
| 9895 | 2P | BG18NUC-HC353 |
| 9896 | 2P | BG18NUC-HC354 |
| 9897 | 2P | BG18NUC-HC355 |
| 9898 | 2P | BG18NUC-HC356 |
| 9899 | 2P | BG18NUC-HC357 |
| 9900 | RBD | BG18NUC-HC358 |
| 9901 | 2P | BG18NUC-HC359 |
| 9902 | 2P | BG18NUC-HC360 |
| 9903 | 2P | BG18NUC-HC361 |
| 9904 | 2P | BG18NUC-HC362 |
| 9905 | 2F | BG18NUC-HC363 |
| 9906 | RBD | BG18NUC-HC364 |
| 9907 | RBD | BG18NUC-HC365 |
| 9908 | 2P | BG18NUC-HC366 |
| 9909 | 2P | BG18NUC-HC367 |
| 9910 | 2P | BG18NUC-HC368 |
| 9911 | 2P | BG18NUC-HC369 |
| 9912 | 2P | BG18NUC-HC370 |
| 9913 | 2P | BG18NUC-HC371 |
| 9914 | 2P | BG18NUC-HC372 |
| 9915 | RBD | BG18NUC-HC373 |
| 9916 | RBD | BG18NUC-HC374 |
| 9917 | RBD | BG18NUC-HC375 |
| 9918 | RBD | BG18NUC-HC376 |
| 9919 | RBD | BG18NUC-HC377 |
| 9920 | 2P | BG18NUC-HC378 |
| 9921 | 2P | BG18NUC-HC379 |
| 9922 | 2P | BG18NUC-HC380 |
| 9923 | RBD | BG18NUC-HC381 |
| 9924 | RBD | BG18NUC-HC382 |
| 9925 | 2P | BG18NUC-HC383 |
| 9926 | RBD | BG18NUC-HC384 |
| 9927 | 2P | BG18NUC-HC385 |
| 9928 | 2P | BG18NUC-HC386 |
| 9929 | 2P | BG18NUC-HC387 |
| 9930 | 2P | BG18NUC-HC388 |
| 9931 | RBD | BG18NUC-HC389 |
| 9932 | 2P | BG18NUC-HC390 |
| 9933 | RBD | BG18NUC-HC391 |
| 9934 | 2P | BG18NUC-HC392 |
| 9935 | 2P | BG18NUC-HC393 |
| 9936 | 2P | BG18NUC-HC394 |
| 9937 | 2P | BG18NUC-HC395 |
| 9938 | 2P | BG18NUC-HC396 |
| 9939 | 2P | BG18NUC-HC397 |
| 9940 | 2P | BG18NUC-HC398 |
| 9941 | 2P | BG18NUC-HC399 |
| 9942 | 2P | BG18NUC-HC400 |
| 9943 | 2P | BG18NUC-HC401 |
| 9944 | 2P | BG18NUC-HC402 |
| 9945 | 2P | BG18NUC-HC403 |
| 9946 | 2P | BG18NUC-HC404 |
| 9947 | RBD | BG18NUC-HC405 |
| 9948 | 2P | BG18NUC-HC406 |
| 9949 | RBD | BG18NUC-HC407 |
| 9950 | RBD | BG18NUC-HC408 |
| 9951 | RBD | BG18NUC-HC409 |
| 9952 | 2P | BG18NUC-HC410 |
| 9953 | 2P | BG18NUC-HC411 |
| 9954 | 2P | BG18NUC-HC412 |
| 9955 | RBD | BG18NUC-HC413 |
| 9956 | RBD | BG18NUC-HC414 |
| 9957 | 2P | BG18NUC-HC415 |
| 9958 | RBD | BG18NUC-HC416 |
| 9959 | 2P | BG18NUC-HC417 |
| 9960 | 2P | BG18NUC-HC418 |
| 9961 | 2P | BG18NUC-HC419 |
| 9962 | RBD | BG18NUC-HC420 |
| 9963 | 2P | BG18NUC-HC421 |
| 9964 | 2P | BG18NUC-HC422 |
| 9965 | 2P | BG18NUC-HC423 |
| 9966 | RBD | BG18NUC-HC424 |
| 9967 | 2P | BG18NUC-HC425 |
| 9968 | 2P | BG18NUC-HC426 |
| 9969 | 2P | BG18NUC-HC427 |
| 9970 | RBD | BG18NUC-HC428 |
| 9971 | 2P | BG18NUC-HC429 |
| 9972 | 2P | BG18NUC-HC430 |
| 9973 | RBD | BG18NUC-HC431 |
| 9974 | 2P | BG18NUC-HC432 |
| 9975 | 2P | BG18NUC-HC433 |
| 9976 | RBD | BG18NUC-HC434 |
| 9977 | RBD | BG18NUC-HC435 |
| 9978 | 2P | BG18NUC-HC436 |
| 9979 | 2P | BG18NUC-HC437 |
| 9980 | 2P | BG18NUC-HC438 |
| 9981 | 2P | BG18NUC-HC439 |
| 9982 | 2P | BG18NUC-HC440 |
| 9983 | 2P | BG18NUC-HC441 |
| 9984 | 2P | BG18NUC-HC442 |
| 9985 | 2P | BG18NUC-HC443 |
| 9986 | 2P | BG18NUC-HC444 |
| 9987 | 2P | BG18NUC-HC445 |
| 9988 | RBD | BG18NUC-HC446 |
| 9989 | 2P | BG18NUC-HC447 |
| 9990 | 2P | BG18NUC-HC448 |
| 9991 | 2P | BG18NUC-HC449 |
| 9992 | 2P | BG18NUC-HC450 |
| 9993 | 2P | BG18NUC-HC451 |
| 9994 | 2P | BG18NUC-HC452 |
| 9995 | 2P | BG18NUC-HC453 |
| 9996 | 2P | BG18NUC-HC454 |
| 9997 | 2P | BG18NUC-HC455 |
| 9998 | 2P | BG18NUC-HC456 |
| 9999 | 2P | BG18NUC-HC457 |
| 10000 | 2P | BG18NUC-HC458 |
| 10001 | 2P | BG18NUC-HC459 |
| 10002 | 2P | BG18NUC-HC460 |
| 10003 | 2P | BG18NUC-HC461 |
| 10004 | RBD | BG18NUC-HC462 |
| 10005 | RBD | BG18NUC-HC463 |
| 10006 | 2P | BG18NUC-HC464 |
| 10007 | 2P | BG18NUC-HC465 |
| 10008 | 2P | BG18NUC-HC466 |
| 10009 | 2P | BG18NUC-HC467 |
| 10010 | RBD | BG18NUC-HC468 |
| 10011 | RBD | BG18NUC-HC469 |
| 10012 | RBD | BG18NUC-HC470 |
| 10013 | 2P | BG18NUC-HC471 |
| 10014 | 2P | BG18NUC-HC472 |
| 10015 | 2P | BG18NUC-HC473 |
| 10016 | 2P | BG18NUC-HC474 |
| 10017 | RBD | BG18NUC-HC475 |
| 10018 | 2P | BG18NUC-HC476 |
| 10019 | RBD | BG18NUC-HC477 |
| 10020 | RBD | BG18NUC-HC478 |
| 10021 | RBD | BG18NUC-HC479 |
| 10022 | RBD | BG18NUC-HC480 |
| 10023 | 2P | BG18NUC-HC481 |
| 10024 | RBD | BG18NUC-HC482 |
| 10025 | 2P | BG18NUC-HC483 |
| 10026 | 2P | BG18NUC-HC484 |
| 10027 | 2P | BG18NUC-HC485 |
| 10028 | 2P | BG18NUC-HC486 |
| 10029 | 2P | BG18NUC-HC487 |
| 10030 | 2P | BG18NUC-HC488 |
| 10031 | 2P | BG18NUC-HC489 |
| 10032 | 2P | BG18NUC-HC490 |
| 10033 | 2P | BG18NUC-HC491 |
| 10034 | RBD | BG18NUC-HC492 |
| 10035 | 2P | BG18NUC-HC493 |
| 10036 | 2P | BG18NUC-HC494 |
| 10037 | 2P | BG18NUC-HC495 |
| 10038 | 2P | BG18NUC-HC496 |
| 10039 | RBD | BG18NUC-HC497 |
| 10040 | 2P | BG18NUC-HC498 |
| 10041 | 2P | BG18NUC-HC499 |
| 10042 | 2P | BG18NUC-HC500 |
| 10043 | 2P | BG18NUC-HC501 |
| 10044 | RBD | BG18NUC-HC502 |
| 10045 | 2P | BG18NUC-HC503 |
| 10046 | 2P | BG18NUC-HC504 |
| 10047 | 2P | BG18NUC-HC505 |
| 10048 | RBD | BG18NUC-HC506 |
| 10049 | 2P | BG18NUC-HC507 |
| 10050 | 2P | BG18NUC-HC508 |
| 10051 | 2P | BG18NUC-HC509 |
| 10052 | 2P | BG18NUC-HC510 |
| 10053 | 2P | BG18NUC-HC511 |
| 10054 | 2P | BG18NUC-HC512 |
| 10055 | 2P | BG18NUC-HC513 |
| 10056 | 2P | BG18NUC-HC514 |
| 10057 | 2P | BG18NUC-HC515 |
| 10058 | RBD | BG18NUC-HC516 |
| 10059 | 2P | BG18NUC-HC517 |
| 10060 | 2P | BG18NUC-HC518 |
| 10061 | 2P | BG18NUC-HC519 |
| 10062 | 2P | BG18NUC-HC520 |
| 10063 | 2P | BG18NUC-HC521 |
| 10064 | RBD | BG18NUC-HC522 |
| 10065 | 2P | BG18NUC-HC523 |
| 10066 | 2P | BG18NUC-HC524 |
| 10067 | 2P | BG18NUC-HC525 |
| 10068 | RBD | BG18NUC-HC526 |
| 10069 | RBD | BG18NUC-HC527 |
| 10070 | 2P | BG18NUC-HC528 |
| 10071 | 2P | BG18NUC-HC529 |
| 10072 | 2P | BG18NUC-HC530 |
| 10073 | 2P | BG18NUC-HC531 |
| 10074 | 2P | BG18NUC-HC532 |
| 10075 | RBD | BG18NUC-HC533 |
| 10076 | 2P | BG18NUC-HC534 |
| 10077 | 2P | BG18NUC-HC535 |
| 10078 | 2P | BG18NUC-HC536 |
| 10079 | 2P | BG18NUC-HC537 |
| 10080 | RBD | BG18NUC-HC538 |
| 10081 | RBD | BG18NUC-HC539 |
| 10082 | 2F | BG18NUC-HC540 |
| 10083 | RBD | BG18NUC-HC541 |
| 10084 | RBD | BG18NUC-HC542 |
| 10085 | RBD | BG18NUC-HC543 |
| 10086 | RBD | BG18NUC-HC544 |
| 10087 | RBD | BG18NUC-HC545 |
| 10088 | RBD | BG18NUC-HC546 |
| 10089 | 2P | BG18NUC-HC547 |
| 10090 | RBD | BG18NUC-HC548 |
| 10091 | RBD | BG18NUC-HC549 |
| 10092 | 2P | BG18NUC-HC550 |
| 10093 | 2P | BG18NUC-HC551 |
| 10094 | 2P | BG18NUC-HC552 |
| 10095 | 2P | BG18NUC-HC553 |
| 10096 | 2P | BG18NUC-HC554 |
| 10097 | 2P | BG18NUC-HC555 |
| 10098 | RBD | BG18NUC-HC556 |
| 10099 | 2P | BG18NUC-HC557 |
| 10100 | RBD | BG18NUC-HC558 |
| 10101 | 2P | BG18NUC-HC559 |
| 10102 | 2P | BG18NUC-HC560 |
| 10103 | 2P | BG18NUC-HC561 |
| 10104 | 2P | BG18NUC-HC562 |
| 10105 | RBD | BG18NUC-HC563 |
| 10106 | 2P | BG18NUC-HC564 |
| 10107 | 2P | BG18NUC-HC565 |
| 10108 | 2P | BG18NUC-HC566 |
| 10109 | 2P | BG18NUC-HC567 |
| 10110 | 2P | BG18NUC-HC568 |
| 10111 | 2P | BG18NUC-HC569 |
| 10112 | 2P | BG18NUC-HC570 |
| 10113 | 2P | BG18NUC-HC571 |
| 10114 | 2P | BG18NUC-HC572 |
| 10115 | 2P | BG18NUC-HC573 |
| 10116 | RBD | BG18NUC-HC574 |
| 10117 | RBD | BG18NUC-HC575 |
| 10118 | 2F | BG18NUC-HC576 |
| 10119 | RBD | BG18NUC-HC577 |
| 10120 | 2P | BG18NUC-HC578 |
| 10121 | 2P | BG18NUC-HC579 |
| 10122 | 2P | BG18NUC-HC580 |
| 10123 | 2P | BG18NUC-HC581 |
| 10124 | 2P | BG18NUC-HC582 |
| 10125 | 2P | BG18NUC-HC583 |
| 10126 | 2P | BG18NUC-HC584 |
| 10127 | 2P | BG18NUC-HC585 |
| 10128 | RBD | BG18NUC-HC586 |
| 10129 | 2P | BG18NUC-HC587 |
| 10130 | 2P | BG18NUC-HC588 |
| 10131 | 2P | BG18NUC-HC589 |
| 10132 | 2P | BG18NUC-HC590 |
| 10133 | 2P | BG18NUC-HC591 |
| 10134 | 2P | BG18NUC-HC592 |
| 10135 | 2P | BG18NUC-HC593 |
| 10136 | 2P | BG18NUC-HC594 |
| 10137 | 2P | BG18NUC-HC595 |
| 10138 | RBD | BG18NUC-HC596 |
| 10139 | 2P | BG18NUC-HC597 |
| 10140 | RBD | BG18NUC-HC598 |
| 10141 | RBD | BG18NUC-HC599 |
| 10142 | RBD | BG18NUC-HC600 |
| 10143 | 2P | BG18NUC-HC601 |
| 10144 | 2P | BG18NUC-HC602 |
| 10145 | 2P | BG18NUC-HC603 |
| 10146 | 2P | BG18NUC-HC604 |
| 10147 | 2P | BG18NUC-HC605 |
| 10148 | RBD | BG18NUC-HC606 |
| 10149 | RBD | BG18NUC-HC607 |
| 10150 | 2P | BG18NUC-HC608 |
| 10151 | RBD | BG18NUC-HC609 |
| 10152 | 2P | BG18NUC-HC610 |
| 10153 | 2P | BG18NUC-HC611 |
| 10154 | 2P | BG18NUC-HC612 |
| 10155 | 2P | BG18NUC-HC613 |
| 10156 | RBD | BG18NUC-HC614 |
| 10157 | 2P | BG18NUC-HC615 |
| 10158 | RBD | BG18NUC-HC616 |
| 10159 | 2P | BG18NUC-HC617 |
| 10160 | 2P | BG18NUC-HC618 |
| 10161 | 2P | BG18NUC-HC619 |
| 10162 | RBD | BG18NUC-HC620 |
| 10163 | 2P | BG18NUC-HC621 |
| 10164 | 2P | BG18NUC-HC622 |
| 10165 | 2P | BG18NUC-HC623 |
| 10166 | 2P | BG18NUC-HC624 |
| 10167 | 2P | BG18NUC-HC625 |
| 10168 | RBD | BG18NUC-HC626 |
| 10169 | 2P | BG18NUC-HC627 |
| 10170 | RBD | BG18NUC-HC628 |
| 10171 | 2P | BG18NUC-HC629 |
| 10172 | 2P | BG18NUC-HC630 |
| 10173 | RBD | BG18NUC-HC631 |
| 10174 | RBD | BG18NUC-HC632 |
| 10175 | 2P | BG18NUC-HC633 |
| 10176 | 2P | BG18NUC-HC634 |
| 10177 | 2P | BG18NUC-HC635 |
| 10178 | 2P | BG18NUC-HC636 |
| 10179 | 2P | BG18NUC-HC637 |
| 10180 | 2P | BG18NUC-HC638 |
| 10181 | RBD | BG18NUC-HC639 |
| 10182 | 2P | BG18NUC-HC640 |
| 10183 | RBD | BG18NUC-HC641 |
| 10184 | 2P | BG18NUC-HC642 |
| 10185 | 2P | BG18NUC-HC643 |
| 10186 | 2P | BG18NUC-HC644 |
| 10187 | 2P | BG18NUC-HC645 |
| 10188 | 2P | BG18NUC-HC646 |
| 10189 | 2P | BG18NUC-HC647 |
| 10190 | RBD | BG18NUC-HC648 |
| 10191 | 2P | BG18NUC-HC649 |
| 10192 | 2P | BG18NUC-HC650 |
| 10193 | 2P | BG18NUC-HC651 |
| 10194 | RBD | BG18NUC-HC652 |
| 10195 | 2P | BG18NUC-HC653 |
| 10196 | RBD | BG18NUC-HC654 |
| 10197 | 2P | BG18NUC-HC655 |
| 10198 | 2P | BG18NUC-HC656 |
| 10199 | 2P | BG18NUC-HC657 |
| 10200 | RBD | BG18NUC-HC658 |
| 10201 | 2P | BG18NUC-HC659 |
| 10202 | 2P | BG18NUC-HC660 |
| 10203 | 2P | BG18NUC-HC661 |
| 10204 | 2P | BG18NUC-HC662 |
| 10205 | RBD | BG18NUC-HC663 |
| 10206 | RBD | BG18NUC-HC664 |
| 10207 | RBD | BG18NUC-HC665 |
| 10208 | RBD | BG18NUC-HC666 |
| 10209 | RBD | BG18NUC-HC667 |
| 10210 | RBD | BG18NUC-HC668 |
| 10211 | RBD | BG18NUC-HC669 |
| 10212 | RBD | BG18NUC-HC670 |
| 10213 | 2P | BG18NUC-HC671 |
| 10214 | 2P | BG18NUC-HC672 |
| 10215 | 2P | BG18NUC-HC673 |
| 10216 | RBD | BG18NUC-HC674 |
| 10217 | 2P | BG18NUC-HC675 |
| 10218 | 2P | BG18NUC-HC676 |
| 10219 | 2P | BG18NUC-HC677 |
| 10220 | RBD | BG18NUC-HC678 |
| 10221 | 2P | BG18NUC-HC679 |
| 10222 | 2P | BG18NUC-HC680 |
| 10223 | 2P | BG18NUC-HC681 |
| 10224 | RBD | BG18NUC-HC682 |
| 10225 | 2P | BG18NUC-HC683 |
| 10226 | 2P | BG18NUC-HC684 |
| 10227 | RBD | BG18NUC-HC685 |
| 10228 | 2P | BG18NUC-HC686 |
| 10229 | 2P | BG18NUC-HC687 |
| 10230 | 2P | BG18NUC-HC688 |
| 10231 | 2P | BG18NUC-HC689 |
| 10232 | 2P | BG18NUC-HC690 |
| 10233 | RBD | BG18NUC-HC691 |
| 10234 | RBD | BG18NUC-HC692 |
| 10235 | RBD | BG18NUC-HC693 |
| 10236 | 2P | BG18NUC-HC694 |
| 10237 | RBD | BG18NUC-HC695 |
| 10238 | 2P | BG18NUC-HC696 |
| 10239 | 2P | BG18NUC-HC697 |
| 10240 | 2P | BG18NUC-HC698 |
| 10241 | 2P | BG18NUC-HC699 |
| 10242 | 2P | BG18NUC-HC700 |
| 10243 | 2P | BG18NUC-HC701 |
| 10244 | 2P | BG18NUC-HC702 |
| 10245 | 2P | BG18NUC-HC703 |
| 10246 | 2P | BG18NUC-HC704 |
| 10247 | 2P | BG18NUC-HC705 |
| 10248 | 2P | BG18NUC-HC706 |
| 10249 | RBD | BG18NUC-HC707 |
| 10250 | 2P | BG18NUC-HC708 |
| 10251 | 2P | BG18NUC-HC709 |
| 10252 | 2P | BG18NUC-HC710 |
| 10253 | RBD | BG18NUC-HC711 |
| 10254 | 2P | BG18NUC-HC712 |
| 10255 | RBD | BG18NUC-HC713 |
| 10256 | RBD | BG18NUC-HC714 |
| 10257 | RBD | BG18NUC-HC715 |
| 10258 | RBD | BG18NUC-HC716 |
| 10259 | RBD | BG18NUC-HC717 |
| 10260 | 2P | BG18NUC-HC718 |
| 10261 | 2P | BG18NUC-HC719 |
| 10262 | RBD | BG18NUC-HC720 |
| 10263 | RBD | BG18NUC-HC721 |
| 10264 | RBD | BG18NUC-HC722 |
| 10265 | 2P | BG18NUC-HC723 |
| 10266 | RBD | BG18NUC-HC724 |
| 10267 | 2P | BG18NUC-HC725 |
| 10268 | 2P | BG18NUC-HC726 |
| 10269 | 2P | BG18NUC-HC727 |
| 10270 | 2P | BG18NUC-HC728 |
| 10271 | 2P | BG18NUC-HC729 |
| 10272 | 2P | BG18NUC-HC730 |
| 10273 | RBD | BG18NUC-HC731 |
| 10274 | RBD | BG18NUC-HC732 |
| 10275 | 2P | BG18NUC-HC733 |
| 10276 | RBD | BG18NUC-HC734 |
| 10277 | 2P | BG18NUC-HC735 |
| 10278 | 2P | BG18NUC-HC736 |
| 10279 | 2P | BG18NUC-HC737 |
| 10280 | 2P | BG18NUC-HC738 |
| 10281 | RBD | BG18NUC-HC739 |
| 10282 | 2P | BG18NUC-HC740 |
| 10283 | 2P | BG18NUC-HC741 |
| 10284 | 2P | BG18NUC-HC742 |
| 10285 | 2F | BG18NUC-HC743 |
| 10286 | RBD | BG18NUC-HC744 |
| 10287 | 2P | BG18NUC-HC745 |
| 10288 | RBD | BG18NUC-HC746 |
| 10289 | RBD | BG18NUC-HC747 |
| 10290 | RBD | BG18NUC-HC748 |
| 10291 | 2P | BG18NUC-HC749 |
| 10292 | 2P | BG18NUC-HC750 |
| 10293 | 2P | BG18NUC-HC751 |
| 10294 | 2P | BG18NUC-LC1 |
| 10295 | 2P | BG18NUC-LC2 |
| 10296 | RBD | BG18NUC-LC3 |
| 10297 | 2P | BG18NUC-LC4 |
| 10298 | 2P | BG18NUC-LC5 |
| 10299 | RBD | BG18NUC-LC6 |
| 10300 | 2P | BG18NUC-LC7 |
| 10301 | 2P | BG18NUC-LC8 |
| 10302 | RBD | BG18NUC-LC9 |
| 10303 | 2P | BG18NUC-LC10 |
| 10304 | 2P | BG18NUC-LC11 |
| 10305 | 2P | BG18NUC-LC12 |
| 10306 | 2P | BG18NUC-LC13 |
| 10307 | 2P | BG18NUC-LC14 |
| 10308 | 2P | BG18NUC-LC15 |
| 10309 | 2P | BG18NUC-LC16 |
| 10310 | RBD | BG18NUC-LC17 |
| 10311 | 2P | BG18NUC-LC18 |
| 10312 | 2P | BG18NUC-LC19 |
| 10313 | RBD | BG18NUC-LC20 |
| 10314 | 2P | BG18NUC-LC21 |
| 10315 | RBD | BG18NUC-LC22 |
| 10316 | 2P | BG18NUC-LC23 |
| 10317 | 2P | BG18NUC-LC24 |
| 10318 | 2P | BG18NUC-LC25 |
| 10319 | 2P | BG18NUC-LC26 |
| 10320 | 2P | BG18NUC-LC27 |
| 10321 | 2P | BG18NUC-LC28 |
| 10322 | 2P | BG18NUC-LC29 |
| 10323 | 2P | BG18NUC-LC30 |
| 10324 | RBD | BG18NUC-LC31 |
| 10325 | RBD | BG18NUC-LC32 |
| 10326 | RBD | BG18NUC-LC33 |
| 10327 | 2P | BG18NUC-LC34 |
| 10328 | 2P | BG18NUC-LC35 |
| 10329 | 2P | BG18NUC-LC36 |
| 10330 | 2P | BG18NUC-LC37 |
| 10331 | 2P | BG18NUC-LC38 |
| 10332 | 2P | BG18NUC-LC39 |
| 10333 | 2P | BG18NUC-LC40 |
| 10334 | RBD | BG18NUC-LC41 |
| 10335 | RBD | BG18NUC-LC42 |
| 10336 | 2P | BG18NUC-LC43 |
| 10337 | 2P | BG18NUC-LC44 |
| 10338 | 2P | BG18NUC-LC45 |
| 10339 | RBD | BG18NUC-LC46 |
| 10340 | 2P | BG18NUC-LC47 |
| 10341 | 2P | BG18NUC-LC48 |
| 10342 | 2P | BG18NUC-LC49 |
| 10343 | 2P | BG18NUC-LC50 |
| 10344 | 2P | BG18NUC-LC51 |
| 10345 | 2P | BG18NUC-LC52 |
| 10346 | 2P | BG18NUC-LC53 |
| 10347 | RBD | BG18NUC-LC54 |
| 10348 | 2P | BG18NUC-LC55 |
| 10349 | 2P | BG18NUC-LC56 |
| 10350 | 2P | BG18NUC-LC57 |
| 10351 | 2P | BG18NUC-LC58 |
| 10352 | RBD | BG18NUC-LC59 |
| 10353 | 2P | BG18NUC-LC60 |
| 10354 | 2P | BG18NUC-LC61 |
| 10355 | 2P | BG18NUC-LC62 |
| 10356 | RBD | BG18NUC-LC63 |
| 10357 | RBD | BG18NUC-LC64 |
| 10358 | 2P | BG18NUC-LC65 |
| 10359 | 2P | BG18NUC-LC66 |
| 10360 | 2P | BG18NUC-LC67 |
| 10361 | 2P | BG18NUC-LC68 |
| 10362 | RBD | BG18NUC-LC69 |
| 10363 | 2P | BG18NUC-LC70 |
| 10364 | RBD | BG18NUC-LC71 |
| 10365 | 2P | BG18NUC-LC72 |
| 10366 | 2P | BG18NUC-LC73 |
| 10367 | 2P | BG18NUC-LC74 |
| 10368 | 2P | BG18NUC-LC75 |
| 10369 | 2P | BG18NUC-LC76 |
| 10370 | RBD | BG18NUC-LC77 |
| 10371 | RBD | BG18NUC-LC78 |
| 10372 | 2P | BG18NUC-LC79 |
| 10373 | 2P | BG18NUC-LC80 |
| 10374 | 2P | BG18NUC-LC81 |
| 10375 | 2P | BG18NUC-LC82 |
| 10376 | 2P | BG18NUC-LC83 |
| 10377 | 2P | BG18NUC-LC84 |
| 10378 | 2P | BG18NUC-LC85 |
| 10379 | 2P | BG18NUC-LC86 |
| 10380 | 2P | BG18NUC-LC87 |
| 10381 | 2P | BG18NUC-LC88 |
| 10382 | 2P | BG18NUC-LC89 |
| 10383 | 2P | BG18NUC-LC90 |
| 10384 | RBD | BG18NUC-LC91 |
| 10385 | RBD | BG18NUC-LC92 |
| 10386 | 2P | BG18NUC-LC93 |
| 10387 | 2P | BG18NUC-LC94 |
| 10388 | 2P | BG18NUC-LC95 |
| 10389 | RBD | BG18NUC-LC96 |
| 10390 | 2P | BG18NUC-LC97 |
| 10391 | 2P | BG18NUC-LC98 |
| 10392 | 2P | BG18NUC-LC99 |
| 10393 | 2P | BG18NUC-LC100 |
| 10394 | RBD | BG18NUC-LC101 |
| 10395 | 2P | BG18NUC-LC102 |
| 10396 | RBD | BG18NUC-LC103 |
| 10397 | RBD | BG18NUC-LC104 |
| 10398 | 2P | BG18NUC-LC105 |
| 10399 | RBD | BG18NUC-LC106 |
| 10400 | RBD | BG18NUC-LC107 |
| 10401 | 2P | BG18NUC-LC108 |
| 10402 | 2P | BG18NUC-LC109 |
| 10403 | RBD | BG18NUC-LC110 |
| 10404 | 2P | BG18NUC-LC111 |
| 10405 | 2P | BG18NUC-LC112 |
| 10406 | 2P | BG18NUC-LC113 |
| 10407 | 2P | BG18NUC-LC114 |
| 10408 | 2P | BG18NUC-LC115 |
| 10409 | RBD | BG18NUC-LC116 |
| 10410 | 2P | BG18NUC-LC117 |
| 10411 | 2P | BG18NUC-LC118 |
| 10412 | 2P | BG18NUC-LC119 |
| 10413 | 2P | BG18NUC-LC120 |
| 10414 | 2P | BG18NUC-LC121 |
| 10415 | 2P | BG18NUC-LC122 |
| 10416 | 2P | BG18NUC-LC123 |
| 10417 | 2P | BG18NUC-LC124 |
| 10418 | 2P | BG18NUC-LC125 |
| 10419 | 2P | BG18NUC-LC126 |
| 10420 | 2P | BG18NUC-LC127 |
| 10421 | 2P | BG18NUC-LC128 |
| 10422 | 2P | BG18NUC-LC129 |
| 10423 | 2P | BG18NUC-LC130 |
| 10424 | 2P | BG18NUC-LC131 |
| 10425 | RBD | BG18NUC-LC132 |
| 10426 | 2P | BG18NUC-LC133 |
| 10427 | RBD | BG18NUC-LC134 |
| 10428 | 2P | BG18NUC-LC135 |
| 10429 | 2P | BG18NUC-LC136 |
| 10430 | RBD | BG18NUC-LC137 |
| 10431 | 2P | BG18NUC-LC138 |
| 10432 | RBD | BG18NUC-LC139 |
| 10433 | RBD | BG18NUC-LC140 |
| 10434 | RBD | BG18NUC-LC141 |
| 10435 | 2P | BG18NUC-LC142 |
| 10436 | 2P | BG18NUC-LC143 |
| 10437 | 2P | BG18NUC-LC144 |
| 10438 | 2P | BG18NUC-LC145 |
| 10439 | 2P | BG18NUC-LC146 |
| 10440 | 2P | BG18NUC-LC147 |
| 10441 | 2P | BG18NUC-LC148 |
| 10442 | 2P | BG18NUC-LC149 |
| 10443 | 2P | BG18NUC-LC150 |
| 10444 | 2P | BG18NUC-LC151 |
| 10445 | 2P | BG18NUC-LC152 |
| 10446 | 2P | BG18NUC-LC153 |
| 10447 | 2P | BG18NUC-LC154 |
| 10448 | RBD | BG18NUC-LC155 |
| 10449 | 2P | BG18NUC-LC156 |
| 10450 | 2P | BG18NUC-LC157 |
| 10451 | 2P | BG18NUC-LC158 |
| 10452 | 2P | BG18NUC-LC159 |
| 10453 | 2P | BG18NUC-LC160 |
| 10454 | 2P | BG18NUC-LC161 |
| 10455 | 2P | BG18NUC-LC162 |
| 10456 | RBD | BG18NUC-LC163 |
| 10457 | 2P | BG18NUC-LC164 |
| 10458 | RBD | BG18NUC-LC165 |
| 10459 | RBD | BG18NUC-LC166 |
| 10460 | 2P | BG18NUC-LC167 |
| 10461 | 2P | BG18NUC-LC168 |
| 10462 | RBD | BG18NUC-LC169 |
| 10463 | 2P | BG18NUC-LC170 |
| 10464 | 2P | BG18NUC-LC171 |
| 10465 | 2P | BG18NUC-LC172 |
| 10466 | 2P | BG18NUC-LC173 |
| 10467 | 2P | BG18NUC-LC174 |
| 10468 | 2P | BG18NUC-LC175 |
| 10469 | 2P | BG18NUC-LC176 |
| 10470 | 2P | BG18NUC-LC177 |
| 10471 | 2P | BG18NUC-LC178 |
| 10472 | 2P | BG18NUC-LC179 |
| 10473 | RBD | BG18NUC-LC180 |
| 10474 | RBD | BG18NUC-LC181 |
| 10475 | 2P | BG18NUC-LC182 |
| 10476 | 2P | BG18NUC-LC183 |
| 10477 | 2P | BG18NUC-LC184 |
| 10478 | 2P | BG18NUC-LC185 |
| 10479 | RBD | BG18NUC-LC186 |
| 10480 | 2P | BG18NUC-LC187 |
| 10481 | 2P | BG18NUC-LC188 |
| 10482 | 2P | BG18NUC-LC189 |
| 10483 | 2P | BG18NUC-LC190 |
| 10484 | 2P | BG18NUC-LC191 |
| 10485 | 2P | BG18NUC-LC192 |
| 10486 | RBD | BG18NUC-LC193 |
| 10487 | 2P | BG18NUC-LC194 |
| 10488 | 2P | BG18NUC-LC195 |
| 10489 | RBD | BG18NUC-LC196 |
| 10490 | RBD | BG18NUC-LC197 |
| 10491 | RBD | BG18NUC-LC198 |
| 10492 | RBD | BG18NUC-LC199 |
| 10493 | 2P | BG18NUC-LC200 |
| 10494 | RBD | BG18NUC-LC201 |
| 10495 | 2P | BG18NUC-LC202 |
| 10496 | RBD | BG18NUC-LC203 |
| 10497 | 2P | BG18NUC-LC204 |
| 10498 | RBD | BG18NUC-LC205 |
| 10499 | 2P | BG18NUC-LC206 |
| 10500 | 2P | BG18NUC-LC207 |
| 10501 | 2P | BG18NUC-LC208 |
| 10502 | 2P | BG18NUC-LC209 |
| 10503 | 2P | BG18NUC-LC210 |
| 10504 | 2P | BG18NUC-LC211 |
| 10505 | RBD | BG18NUC-LC212 |
| 10506 | 2P | BG18NUC-LC213 |
| 10507 | RBD | BG18NUC-LC214 |
| 10508 | RBD | BG18NUC-LC215 |
| 10509 | RBD | BG18NUC-LC216 |
| 10510 | RBD | BG18NUC-LC217 |
| 10511 | 2P | BG18NUC-LC218 |
| 10512 | RBD | BG18NUC-LC219 |
| 10513 | 2P | BG18NUC-LC220 |
| 10514 | 2P | BG18NUC-LC221 |
| 10515 | 2P | BG18NUC-LC222 |
| 10516 | 2P | BG18NUC-LC223 |
| 10517 | RBD | BG18NUC-LC224 |
| 10518 | RBD | BG18NUC-LC225 |
| 10519 | 2P | BG18NUC-LC226 |
| 10520 | 2P | BG18NUC-LC227 |
| 10521 | 2P | BG18NUC-LC228 |
| 10522 | 2P | BG18NUC-LC229 |
| 10523 | RBD | BG18NUC-LC230 |
| 10524 | 2P | BG18NUC-LC231 |
| 10525 | RBD | BG18NUC-LC232 |
| 10526 | 2P | BG18NUC-LC233 |
| 10527 | 2P | BG18NUC-LC234 |
| 10528 | 2P | BG18NUC-LC235 |
| 10529 | 2P | BG18NUC-LC236 |
| 10530 | 2P | BG18NUC-LC237 |
| 10531 | RBD | BG18NUC-LC238 |
| 10532 | RBD | BG18NUC-LC239 |
| 10533 | RBD | BG18NUC-LC240 |
| 10534 | 2P | BG18NUC-LC241 |
| 10535 | RBD | BG18NUC-LC242 |
| 10536 | 2P | BG18NUC-LC243 |
| 10537 | 2P | BG18NUC-LC244 |
| 10538 | 2P | BG18NUC-LC245 |
| 10539 | 2P | BG18NUC-LC246 |
| 10540 | 2P | BG18NUC-LC247 |
| 10541 | 2P | BG18NUC-LC248 |
| 10542 | 2P | BG18NUC-LC249 |
| 10543 | 2P | BG18NUC-LC250 |
| 10544 | 2P | BG18NUC-LC251 |
| 10545 | RBD | BG18NUC-LC252 |
| 10546 | 2P | BG18NUC-LC253 |
| 10547 | RBD | BG18NUC-LC254 |
| 10548 | RBD | BG18NUC-LC255 |
| 10549 | 2P | BG18NUC-LC256 |
| 10550 | 2P | BG18NUC-LC257 |
| 10551 | 2F | BG18NUC-LC258 |
| 10552 | RBD | BG18NUC-LC259 |
| 10553 | 2P | BG18NUC-LC260 |
| 10554 | RBD | BG18NUC-LC261 |
| 10555 | 2P | BG18NUC-LC262 |
| 10556 | RBD | BG18NUC-LC263 |
| 10557 | 2P | BG18NUC-LC264 |
| 10558 | 2P | BG18NUC-LC265 |
| 10559 | 2P | BG18NUC-LC266 |
| 10560 | RBD | BG18NUC-LC267 |
| 10561 | 2P | BG18NUC-LC268 |
| 10562 | 2P | BG18NUC-LC269 |
| 10563 | 2P | BG18NUC-LC270 |
| 10564 | 2F | BG18NUC-LC271 |
| 10565 | 2P | BG18NUC-LC272 |
| 10566 | 2P | BG18NUC-LC273 |
| 10567 | 2P | BG18NUC-LC274 |
| 10568 | 2P | BG18NUC-LC275 |
| 10569 | 2P | BG18NUC-LC276 |
| 10570 | 2P | BG18NUC-LC277 |
| 10571 | 2P | BG18NUC-LC278 |
| 10572 | RBD | BG18NUC-LC279 |
| 10573 | 2P | BG18NUC-LC280 |
| 10574 | 2P | BG18NUC-LC281 |
| 10575 | RBD | BG18NUC-LC282 |
| 10576 | 2P | BG18NUC-LC283 |
| 10577 | RBD | BG18NUC-LC284 |
| 10578 | 2P | BG18NUC-LC285 |
| 10579 | RBD | BG18NUC-LC286 |
| 10580 | RBD | BG18NUC-LC287 |
| 10581 | 2P | BG18NUC-LC288 |
| 10582 | 2P | BG18NUC-LC289 |
| 10583 | 2P | BG18NUC-LC290 |
| 10584 | RBD | BG18NUC-LC291 |
| 10585 | RBD | BG18NUC-LC292 |
| 10586 | RBD | BG18NUC-LC293 |
| 10587 | 2P | BG18NUC-LC294 |
| 10588 | 2P | BG18NUC-LC295 |
| 10589 | 2P | BG18NUC-LC296 |
| 10590 | 2P | BG18NUC-LC297 |
| 10591 | 2P | BG18NUC-LC298 |
| 10592 | RBD | BG18NUC-LC299 |
| 10593 | 2P | BG18NUC-LC300 |
| 10594 | 2P | BG18NUC-LC301 |
| 10595 | 2P | BG18NUC-LC302 |
| 10596 | 2P | BG18NUC-LC303 |
| 10597 | 2P | BG18NUC-LC304 |
| 10598 | 2P | BG18NUC-LC305 |
| 10599 | 2P | BG18NUC-LC306 |
| 10600 | 2P | BG18NUC-LC307 |
| 10601 | 2P | BG18NUC-LC308 |
| 10602 | RBD | BG18NUC-LC309 |
| 10603 | 2P | BG18NUC-LC310 |
| 10604 | RBD | BG18NUC-LC311 |
| 10605 | 2P | BG18NUC-LC312 |
| 10606 | 2P | BG18NUC-LC313 |
| 10607 | 2P | BG18NUC-LC314 |
| 10608 | RBD | BG18NUC-LC315 |
| 10609 | 2P | BG18NUC-LC316 |
| 10610 | 2P | BG18NUC-LC317 |
| 10611 | 2P | BG18NUC-LC318 |
| 10612 | 2P | BG18NUC-LC319 |
| 10613 | 2P | BG18NUC-LC320 |
| 10614 | 2P | BG18NUC-LC321 |
| 10615 | RBD | BG18NUC-LC322 |
| 10616 | RBD | BG18NUC-LC323 |
| 10617 | RBD | BG18NUC-LC324 |
| 10618 | RBD | BG18NUC-LC325 |
| 10619 | 2P | BG18NUC-LC326 |
| 10620 | 2P | BG18NUC-LC327 |
| 10621 | RBD | BG18NUC-LC328 |
| 10622 | 2P | BG18NUC-LC329 |
| 10623 | 2P | BG18NUC-LC330 |
| 10624 | 2P | BG18NUC-LC331 |
| 10625 | 2P | BG18NUC-LC332 |
| 10626 | 2P | BG18NUC-LC333 |
| 10627 | RBD | BG18NUC-LC334 |
| 10628 | 2P | BG18NUC-LC335 |
| 10629 | RBD | BG18NUC-LC336 |
| 10630 | RBD | BG18NUC-LC337 |
| 10631 | 2P | BG18NUC-LC338 |
| 10632 | 2P | BG18NUC-LC339 |
| 10633 | 2P | BG18NUC-LC340 |
| 10634 | RBD | BG18NUC-LC341 |
| 10635 | 2P | BG18NUC-LC342 |
| 10636 | RBD | BG18NUC-LC343 |
| 10637 | 2P | BG18NUC-LC344 |
| 10638 | 2P | BG18NUC-LC345 |
| 10639 | 2P | BG18NUC-LC346 |
| 10640 | 2P | BG18NUC-LC347 |
| 10641 | 2P | BG18NUC-LC348 |
| 10642 | 2P | BG18NUC-LC349 |
| 10643 | 2P | BG18NUC-LC350 |
| 10644 | RBD | BG18NUC-LC351 |
| 10645 | 2P | BG18NUC-LC352 |
| 10646 | 2P | BG18NUC-LC353 |
| 10647 | 2P | BG18NUC-LC354 |
| 10648 | 2P | BG18NUC-LC355 |
| 10649 | 2P | BG18NUC-LC356 |
| 10650 | 2P | BG18NUC-LC357 |
| 10651 | RBD | BG18NUC-LC358 |
| 10652 | 2P | BG18NUC-LC359 |
| 10653 | 2P | BG18NUC-LC360 |
| 10654 | 2P | BG18NUC-LC361 |
| 10655 | 2P | BG18NUC-LC362 |
| 10656 | 2P | BG18NUC-LC363 |
| 10657 | RBD | BG18NUC-LC364 |
| 10658 | RBD | BG18NUC-LC365 |
| 10659 | 2P | BG18NUC-LC366 |
| 10660 | 2P | BG18NUC-LC367 |
| 10661 | 2P | BG18NUC-LC368 |
| 10662 | 2P | BG18NUC-LC369 |
| 10663 | 2P | BG18NUC-LC370 |
| 10664 | 2P | BG18NUC-LC371 |
| 10665 | 2P | BG18NUC-LC372 |
| 10666 | RBD | BG18NUC-LC373 |
| 10667 | RBD | BG18NUC-LC374 |
| 10668 | RBD | BG18NUC-LC375 |
| 10669 | RBD | BG18NUC-LC376 |
| 10670 | RBD | BG18NUC-LC377 |
| 10671 | 2P | BG18NUC-LC378 |
| 10672 | 2P | BG18NUC-LC379 |
| 10673 | 2P | BG18NUC-LC380 |
| 10674 | RBD | BG18NUC-LC381 |
| 10675 | RBD | BG18NUC-LC382 |
| 10676 | 2P | BG18NUC-LC383 |
| 10677 | RBD | BG18NUC-LC384 |
| 10678 | 2P | BG18NUC-LC385 |
| 10679 | 2P | BG18NUC-LC386 |
| 10680 | 2P | BG18NUC-LC387 |
| 10681 | 2P | BG18NUC-LC388 |
| 10682 | RBD | BG18NUC-LC389 |
| 10683 | 2P | BG18NUC-LC390 |
| 10684 | RBD | BG18NUC-LC391 |
| 10685 | 2P | BG18NUC-LC392 |
| 10686 | 2P | BG18NUC-LC393 |
| 10687 | 2P | BG18NUC-LC394 |
| 10688 | 2P | BG18NUC-LC395 |
| 10689 | 2P | BG18NUC-LC396 |
| 10690 | 2P | BG18NUC-LC397 |
| 10691 | 2P | BG18NUC-LC398 |
| 10692 | 2P | BG18NUC-LC399 |
| 10693 | 2P | BG18NUC-LC400 |
| 10694 | 2P | BG18NUC-LC401 |
| 10695 | 2P | BG18NUC-LC402 |
| 10696 | 2P | BG18NUC-LC403 |
| 10697 | 2F | BG18NUC-LC404 |
| 10698 | RBD | BG18NUC-LC405 |
| 10699 | 2P | BG18NUC-LC406 |
| 10700 | RBD | BG18NUC-LC407 |
| 10701 | RBD | BG18NUC-LC408 |
| 10702 | RBD | BG18NUC-LC409 |
| 10703 | 2P | BG18NUC-LC410 |
| 10704 | 2P | BG18NUC-LC411 |
| 10705 | 2P | BG18NUC-LC412 |
| 10706 | RBD | BG18NUC-LC413 |
| 10707 | RBD | BG18NUC-LC414 |
| 10708 | 2P | BG18NUC-LC415 |
| 10709 | RBD | BG18NUC-LC416 |
| 10710 | 2P | BG18NUC-LC417 |
| 10711 | 2P | BG18NUC-LC418 |
| 10712 | 2P | BG18NUC-LC419 |
| 10713 | RBD | BG18NUC-LC420 |
| 10714 | 2P | BG18NUC-LC421 |
| 10715 | 2P | BG18NUC-LC422 |
| 10716 | 2P | BG18NUC-LC423 |
| 10717 | RBD | BG18NUC-LC424 |
| 10718 | 2P | BG18NUC-LC425 |
| 10719 | 2P | BG18NUC-LC426 |
| 10720 | 2P | BG18NUC-LC427 |
| 10721 | RBD | BG18NUC-LC428 |
| 10722 | 2P | BG18NUC-LC429 |
| 10723 | 2F | BG18NUC-LC430 |
| 10724 | RBD | BG18NUC-LC431 |
| 10725 | 2P | BG18NUC-LC432 |
| 10726 | 2P | BG18NUC-LC433 |
| 10727 | RBD | BG18NUC-LC434 |
| 10728 | RBD | BG18NUC-LC435 |
| 10729 | 2P | BG18NUC-LC436 |
| 10730 | 2P | BG18NUC-LC437 |
| 10731 | 2P | BG18NUC-LC438 |
| 10732 | 2P | BG18NUC-LC439 |
| 10733 | 2P | BG18NUC-LC440 |
| 10734 | 2P | BG18NUC-LC441 |
| 10735 | 2P | BG18NUC-LC442 |
| 10736 | 2P | BG18NUC-LC443 |
| 10737 | 2F | BG18NUC-LC444 |
| 10738 | 2P | BG18NUC-LC445 |
| 10739 | RBD | BG18NUC-LC446 |
| 10740 | 2P | BG18NUC-LC447 |
| 10741 | 2P | BG18NUC-LC448 |
| 10742 | 2F | BG18NUC-LC449 |
| 10743 | 2P | BG18NUC-LC450 |
| 10744 | 2P | BG18NUC-LC451 |
| 10745 | 2P | BG18NUC-LC452 |
| 10746 | 2P | BG18NUC-LC453 |
| 10747 | 2P | BG18NUC-LC454 |
| 10748 | 2F | BG18NUC-LC455 |
| 10749 | 2P | BG18NUC-LC456 |
| 10750 | 2P | BG18NUC-LC457 |
| 10751 | 2P | BG18NUC-LC458 |
| 10752 | 2P | BG18NUC-LC459 |
| 10753 | 2P | BG18NUC-LC460 |
| 10754 | 2P | BG18NUC-LC461 |
| 10755 | RBD | BG18NUC-LC462 |
| 10756 | RBD | BG18NUC-LC463 |
| 10757 | 2P | BG18NUC-LC464 |
| 10758 | 2F | BG18NUC-LC465 |
| 10759 | 2F | BG18NUC-LC466 |
| 10760 | 2F | BG18NUC-LC467 |
| 10761 | RBD | BG18NUC-LC468 |
| 10762 | RBD | BG18NUC-LC469 |
| 10763 | RBD | BG18NUC-LC470 |
| 10764 | 2P | BG18NUC-LC471 |
| 10765 | 2P | BG18NUC-LC472 |
| 10766 | 2P | BG18NUC-LC473 |
| 10767 | 2P | BG18NUC-LC474 |
| 10768 | RBD | BG18NUC-LC475 |
| 10769 | 2P | BG18NUC-LC476 |
| 10770 | RBD | BG18NUC-LC477 |
| 10771 | RBD | BG18NUC-LC478 |
| 10772 | RBD | BG18NUC-LC479 |
| 10773 | RBD | BG18NUC-LC480 |
| 10774 | 2P | BG18NUC-LC481 |
| 10775 | RBD | BG18NUC-LC482 |
| 10776 | 2P | BG18NUC-LC483 |
| 10777 | 2P | BG18NUC-LC484 |
| 10778 | 2P | BG18NUC-LC485 |
| 10779 | 2P | BG18NUC-LC486 |
| 10780 | 2P | BG18NUC-LC487 |
| 10781 | 2P | BG18NUC-LC488 |
| 10782 | 2P | BG18NUC-LC489 |
| 10783 | 2P | BG18NUC-LC490 |
| 10784 | 2P | BG18NUC-LC491 |
| 10785 | RBD | BG18NUC-LC492 |
| 10786 | 2P | BG18NUC-LC493 |
| 10787 | 2F | BG18NUC-LC494 |
| 10788 | 2P | BG18NUC-LC495 |
| 10789 | 2P | BG18NUC-LC496 |
| 10790 | RBD | BG18NUC-LC497 |
| 10791 | 2F | BG18NUC-LC498 |
| 10792 | 2F | BG18NUC-LC499 |
| 10793 | 2P | BG18NUC-LC500 |
| 10794 | 2P | BG18NUC-LC501 |
| 10795 | RBD | BG18NUC-LC502 |
| 10796 | 2P | BG18NUC-LC503 |
| 10797 | 2F | BG18NUC-LC504 |
| 10798 | 2P | BG18NUC-LC505 |
| 10799 | RBD | BG18NUC-LC506 |
| 10800 | 2P | BG18NUC-LC507 |
| 10801 | 2P | BG18NUC-LC508 |
| 10802 | 2F | BG18NUC-LC509 |
| 10803 | 2P | BG18NUC-LC510 |
| 10804 | 2P | BG18NUC-LC511 |
| 10805 | 2P | BG18NUC-LC512 |
| 10806 | 2P | BG18NUC-LC513 |
| 10807 | 2P | BG18NUC-LC514 |
| 10808 | 2P | BG18NUC-LC515 |
| 10809 | RBD | BG18NUC-LC516 |
| 10810 | 2P | BG18NUC-LC517 |
| 10811 | 2P | BG18NUC-LC518 |
| 10812 | 2P | BG18NUC-LC519 |
| 10813 | 2P | BG18NUC-LC520 |
| 10814 | 2P | BG18NUC-LC521 |
| 10815 | RBD | BG18NUC-LC522 |
| 10816 | 2P | BG18NUC-LC523 |
| 10817 | 2P | BG18NUC-LC524 |
| 10818 | 2P | BG18NUC-LC525 |
| 10819 | RBD | BG18NUC-LC526 |
| 10820 | RBD | BG18NUC-LC527 |
| 10821 | 2P | BG18NUC-LC528 |
| 10822 | 2P | BG18NUC-LC529 |
| 10823 | 2P | BG18NUC-LC530 |
| 10824 | 2P | BG18NUC-LC531 |
| 10825 | 2P | BG18NUC-LC532 |
| 10826 | RBD | BG18NUC-LC533 |
| 10827 | 2P | BG18NUC-LC534 |
| 10828 | 2P | BG18NUC-LC535 |
| 10829 | 2P | BG18NUC-LC536 |
| 10830 | 2P | BG18NUC-LC537 |
| 10831 | RBD | BG18NUC-LC538 |
| 10832 | RBD | BG18NUC-LC539 |
| 10833 | 2P | BG18NUC-LC540 |
| 10834 | RBD | BG18NUC-LC541 |
| 10835 | RBD | BG18NUC-LC542 |
| 10836 | RBD | BG18NUC-LC543 |
| 10837 | RBD | BG18NUC-LC544 |
| 10838 | RBD | BG18NUC-LC545 |
| 10839 | RBD | BG18NUC-LC546 |
| 10840 | 2P | BG18NUC-LC547 |
| 10841 | RBD | BG18NUC-LC548 |
| 10842 | RBD | BG18NUC-LC549 |
| 10843 | 2P | BG18NUC-LC550 |
| 10844 | 2P | BG18NUC-LC551 |
| 10845 | 2P | BG18NUC-LC552 |
| 10846 | 2P | BG18NUC-LC553 |
| 10847 | 2P | BG18NUC-LC554 |
| 10848 | 2P | BG18NUC-LC555 |
| 10849 | RBD | BG18NUC-LC556 |
| 10850 | 2P | BG18NUC-LC557 |
| 10851 | RBD | BG18NUC-LC558 |
| 10852 | 2P | BG18NUC-LC559 |
| 10853 | 2P | BG18NUC-LC560 |
| 10854 | 2P | BG18NUC-LC561 |
| 10855 | 2P | BG18NUC-LC562 |
| 10856 | RBD | BG18NUC-LC563 |
| 10857 | 2P | BG18NUC-LC564 |
| 10858 | 2P | BG18NUC-LC565 |
| 10859 | 2P | BG18NUC-LC566 |
| 10860 | 2P | BG18NUC-LC567 |
| 10861 | 2P | BG18NUC-LC568 |
| 10862 | 2P | BG18NUC-LC569 |
| 10863 | 2P | BG18NUC-LC570 |
| 10864 | 2P | BG18NUC-LC571 |
| 10865 | 2P | BG18NUC-LC572 |
| 10866 | 2P | BG18NUC-LC573 |
| 10867 | RBD | BG18NUC-LC574 |
| 10868 | RBD | BG18NUC-LC575 |
| 10869 | 2P | BG18NUC-LC576 |
| 10870 | RBD | BG18NUC-LC577 |
| 10871 | 2P | BG18NUC-LC578 |
| 10872 | 2P | BG18NUC-LC579 |
| 10873 | 2P | BG18NUC-LC580 |
| 10874 | 2P | BG18NUC-LC581 |
| 10875 | 2P | BG18NUC-LC582 |
| 10876 | 2P | BG18NUC-LC583 |
| 10877 | 2P | BG18NUC-LC584 |
| 10878 | 2P | BG18NUC-LC585 |
| 10879 | RBD | BG18NUC-LC586 |
| 10880 | 2P | BG18NUC-LC587 |
| 10881 | 2P | BG18NUC-LC588 |
| 10882 | 2P | BG18NUC-LC589 |
| 10883 | 2P | BG18NUC-LC590 |
| 10884 | 2P | BG18NUC-LC591 |
| 10885 | 2P | BG18NUC-LC592 |
| 10886 | 2P | BG18NUC-LC593 |
| 10887 | 2P | BG18NUC-LC594 |
| 10888 | 2P | BG18NUC-LC595 |
| 10889 | RBD | BG18NUC-LC596 |
| 10890 | 2P | BG18NUC-LC597 |
| 10891 | RBD | BG18NUC-LC598 |
| 10892 | RBD | BG18NUC-LC599 |
| 10893 | RBD | BG18NUC-LC600 |
| 10894 | 2P | BG18NUC-LC601 |
| 10895 | 2P | BG18NUC-LC602 |
| 10896 | 2P | BG18NUC-LC603 |
| 10897 | 2P | BG18NUC-LC604 |
| 10898 | 2P | BG18NUC-LC605 |
| 10899 | RBD | BG18NUC-LC606 |
| 10900 | RBD | BG18NUC-LC607 |
| 10901 | 2P | BG18NUC-LC608 |
| 10902 | RBD | BG18NUC-LC609 |
| 10903 | 2P | BG18NUC-LC610 |
| 10904 | 2P | BG18NUC-LC611 |
| 10905 | 2P | BG18NUC-LC612 |
| 10906 | 2P | BG18NUC-LC613 |
| 10907 | RBD | BG18NUC-LC614 |
| 10908 | 2P | BG18NUC-LC615 |
| 10909 | RBD | BG18NUC-LC616 |
| 10910 | 2P | BG18NUC-LC617 |
| 10911 | 2P | BG18NUC-LC618 |
| 10912 | 2P | BG18NUC-LC619 |
| 10913 | RBD | BG18NUC-LC620 |
| 10914 | 2P | BG18NUC-LC621 |
| 10915 | 2P | BG18NUC-LC622 |
| 10916 | 2P | BG18NUC-LC623 |
| 10917 | 2P | BG18NUC-LC624 |
| 10918 | 2P | BG18NUC-LC625 |
| 10919 | RBD | BG18NUC-LC626 |
| 10920 | 2P | BG18NUC-LC627 |
| 10921 | RBD | BG18NUC-LC628 |
| 10922 | 2P | BG18NUC-LC629 |
| 10923 | 2P | BG18NUC-LC630 |
| 10924 | RBD | BG18NUC-LC631 |
| 10925 | RBD | BG18NUC-LC632 |
| 10926 | 2P | BG18NUC-LC633 |
| 10927 | 2P | BG18NUC-LC634 |
| 10928 | 2P | BG18NUC-LC635 |
| 10929 | 2P | BG18NUC-LC636 |
| 10930 | 2P | BG18NUC-LC637 |
| 10931 | 2P | BG18NUC-LC638 |
| 10932 | RBD | BG18NUC-LC639 |
| 10933 | 2P | BG18NUC-LC640 |
| 10934 | RBD | BG18NUC-LC641 |
| 10935 | 2P | BG18NUC-LC642 |
| 10936 | 2P | BG18NUC-LC643 |
| 10937 | 2P | BG18NUC-LC644 |
| 10938 | 2P | BG18NUC-LC645 |
| 10939 | 2P | BG18NUC-LC646 |
| 10940 | 2P | BG18NUC-LC647 |
| 10941 | RBD | BG18NUC-LC648 |
| 10942 | 2P | BG18NUC-LC649 |
| 10943 | 2P | BG18NUC-LC650 |
| 10944 | 2F | BG18NUC-LC651 |
| 10945 | RBD | BG18NUC-LC652 |
| 10946 | 2P | BG18NUC-LC653 |
| 10947 | RBD | BG18NUC-LC654 |
| 10948 | 2P | BG18NUC-LC655 |
| 10949 | 2P | BG18NUC-LC656 |
| 10950 | 2P | BG18NUC-LC657 |
| 10951 | RBD | BG18NUC-LC658 |
| 10952 | 2P | BG18NUC-LC659 |
| 10953 | 2P | BG18NUC-LC660 |
| 10954 | 2P | BG18NUC-LC661 |
| 10955 | 2P | BG18NUC-LC662 |
| 10956 | RBD | BG18NUC-LC663 |
| 10957 | RBD | BG18NUC-LC664 |
| 10958 | RBD | BG18NUC-LC665 |
| 10959 | RBD | BG18NUC-LC666 |
| 10960 | RBD | BG18NUC-LC667 |
| 10961 | RBD | BG18NUC-LC668 |
| 10962 | RBD | BG18NUC-LC669 |
| 10963 | RBD | BG18NUC-LC670 |
| 10964 | 2P | BG18NUC-LC671 |
| 10965 | 2P | BG18NUC-LC672 |
| 10966 | 2P | BG18NUC-LC673 |
| 10967 | RBD | BG18NUC-LC674 |
| 10968 | 2P | BG18NUC-LC675 |
| 10969 | 2P | BG18NUC-LC676 |
| 10970 | 2P | BG18NUC-LC677 |
| 10971 | RBD | BG18NUC-LC678 |
| 10972 | 2P | BG18NUC-LC679 |
| 10973 | 2P | BG18NUC-LC680 |
| 10974 | 2P | BG18NUC-LC681 |
| 10975 | RBD | BG18NUC-LC682 |
| 10976 | 2P | BG18NUC-LC683 |
| 10977 | 2P | BG18NUC-LC684 |
| 10978 | RBD | BG18NUC-LC685 |
| 10979 | 2P | BG18NUC-LC686 |
| 10980 | 2P | BG18NUC-LC687 |
| 10981 | 2P | BG18NUC-LC688 |
| 10982 | 2P | BG18NUC-LC689 |
| 10983 | 2P | BG18NUC-LC690 |
| 10984 | RBD | BG18NUC-LC691 |
| 10985 | RBD | BG18NUC-LC692 |
| 10986 | RBD | BG18NUC-LC693 |
| 10987 | 2P | BG18NUC-LC694 |
| 10988 | RBD | BG18NUC-LC695 |
| 10989 | 2P | BG18NUC-LC696 |
| 10990 | 2P | BG18NUC-LC697 |
| 10991 | 2P | BG18NUC-LC698 |
| 10992 | 2P | BG18NUC-LC699 |
| 10993 | 2P | BG18NUC-LC700 |
| 10994 | 2P | BG18NUC-LC701 |
| 10995 | 2P | BG18NUC-LC702 |
| 10996 | 2P | BG18NUC-LC703 |
| 10997 | 2P | BG18NUC-LC704 |
| 10998 | 2P | BG18NUC-LC705 |
| 10999 | 2P | BG18NUC-LC706 |
| 11000 | RBD | BG18NUC-LC707 |
| 11001 | 2P | BG18NUC-LC708 |
| 11002 | 2P | BG18NUC-LC709 |
| 11003 | 2P | BG18NUC-LC710 |
| 11004 | RBD | BG18NUC-LC711 |
| 11005 | 2P | BG18NUC-LC712 |
| 11006 | RBD | BG18NUC-LC713 |
| 11007 | RBD | BG18NUC-LC714 |
| 11008 | RBD | BG18NUC-LC715 |
| 11009 | RBD | BG18NUC-LC716 |
| 11010 | RBD | BG18NUC-LC717 |
| 11011 | 2P | BG18NUC-LC718 |
| 11012 | 2P | BG18NUC-LC719 |
| 11013 | RBD | BG18NUC-LC720 |
| 11014 | RBD | BG18NUC-LC721 |
| 11015 | RBD | BG18NUC-LC722 |
| 11016 | 2P | BG18NUC-LC723 |
| 11017 | RBD | BG18NUC-LC724 |
| 11018 | 2P | BG18NUC-LC725 |
| 11019 | 2P | BG18NUC-LC726 |
| 11020 | 2P | BG18NUC-LC727 |
| 11021 | 2P | BG18NUC-LC728 |
| 11022 | 2P | BG18NUC-LC729 |
| 11023 | 2P | BG18NUC-LC730 |
| 11024 | RBD | BG18NUC-LC731 |
| 11025 | RBD | BG18NUC-LC732 |
| 11026 | 2P | BG18NUC-LC733 |
| 11027 | RBD | BG18NUC-LC734 |
| 11028 | 2P | BG18NUC-LC735 |
| 11029 | 2P | BG18NUC-LC736 |
| 11030 | 2P | BG18NUC-LC737 |
| 11031 | 2P | BG18NUC-LC738 |
| 11032 | RBD | BG18NUC-LC739 |
| 11033 | 2P | BG18NUC-LC740 |
| 11034 | 2P | BG18NUC-LC741 |
| 11035 | 2P | BG18NUC-LC742 |
| 11036 | 2P | BG18NUC-LC743 |
| 11037 | RBD | BG18NUC-LC744 |
| 11038 | 2P | BG18NUC-LC745 |
| 11039 | RBD | BG18NUC-LC746 |
| 11040 | RBD | BG18NUC-LC747 |
| 11041 | RBD | BG18NUC-LC748 |
| 11042 | 2P | BG18NUC-LC749 |
| 11043 | 2P | BG18NUC-LC750 |
| 11044 | 2P | BG18NUC-LC751 |
| 11045 | 2P | BG19NUC-HC1 |
| 11046 | RBD | BG19NUC-HC2 |
| 11047 | RBD | BG19NUC-HC3 |
| 11048 | RBD | BG19NUC-HC4 |
| 11049 | 2P | BG19NUC-HC5 |
| 11050 | 2P | BG19NUC-HC6 |
| 11051 | RBD | BG19NUC-HC7 |
| 11052 | RBD | BG19NUC-HC8 |
| 11053 | 2P | BG19NUC-HC9 |
| 11054 | 2P | BG19NUC-HC10 |
| 11055 | 2P | BG19NUC-HC11 |
| 11056 | RBD | BG19NUC-HC12 |
| 11057 | RBD | BG19NUC-HC13 |
| 11058 | 2P | BG19NUC-HC14 |
| 11059 | RBD | BG19NUC-HC15 |
| 11060 | RBD | BG19NUC-HC16 |
| 11061 | 2P | BG19NUC-HC17 |
| 11062 | 2P | BG19NUC-HC18 |
| 11063 | 2P | BG19NUC-HC19 |
| 11064 | 2P | BG19NUC-HC20 |
| 11065 | 2P | BG19NUC-HC21 |
| 11066 | 2P | BG19NUC-HC22 |
| 11067 | RBD | BG19NUC-HC23 |
| 11068 | 2P | BG19NUC-HC24 |
| 11069 | 2P | BG19NUC-HC25 |
| 11070 | RBD | BG19NUC-HC26 |
| 11071 | 2P | BG19NUC-HC27 |
| 11072 | RBD | BG19NUC-HC28 |
| 11073 | 2P | BG19NUC-HC29 |
| 11074 | RBD | BG19NUC-HC30 |
| 11075 | 2P | BG19NUC-HC31 |
| 11076 | 2P | BG19NUC-HC32 |
| 11077 | 2P | BG19NUC-HC33 |
| 11078 | RBD | BG19NUC-HC34 |
| 11079 | 2P | BG19NUC-HC35 |
| 11080 | RBD | BG19NUC-HC36 |
| 11081 | RBD | BG19NUC-HC37 |
| 11082 | 2P | BG19NUC-HC38 |
| 11083 | 2P | BG19NUC-HC39 |
| 11084 | RBD | BG19NUC-HC40 |
| 11085 | 2P | BG19NUC-HC41 |
| 11086 | 2P | BG19NUC-HC42 |
| 11087 | 2P | BG19NUC-HC43 |
| 11088 | 2P | BG19NUC-HC44 |
| 11089 | 2P | BG19NUC-HC45 |
| 11090 | 2P | BG19NUC-HC46 |
| 11091 | RBD | BG19NUC-HC47 |
| 11092 | RBD | BG19NUC-HC48 |
| 11093 | RBD | BG19NUC-HC49 |
| 11094 | 2P | BG19NUC-HC50 |
| 11095 | 2P | BG19NUC-HC51 |
| 11096 | 2P | BG19NUC-HC52 |
| 11097 | 2F | BG19NUC-HC53 |
| 11098 | RBD | BG19NUC-HC54 |
| 11099 | 2P | BG19NUC-HC55 |
| 11100 | 2P | BG19NUC-HC56 |
| 11101 | 2P | BG19NUC-HC57 |
| 11102 | 2P | BG19NUC-HC58 |
| 11103 | RBD | BG19NUC-HC59 |
| 11104 | RBD | BG19NUC-HC60 |
| 11105 | RBD | BG19NUC-HC61 |
| 11106 | 2P | BG19NUC-HC62 |
| 11107 | 2P | BG19NUC-HC63 |
| 11108 | RBD | BG19NUC-HC64 |
| 11109 | 2P | BG19NUC-HC65 |
| 11110 | 2P | BG19NUC-HC66 |
| 11111 | 2P | BG19NUC-HC67 |
| 11112 | 2P | BG19NUC-HC68 |
| 11113 | 2P | BG19NUC-HC69 |
| 11114 | 2P | BG19NUC-HC70 |
| 11115 | 2F | BG19NUC-HC71 |
| 11116 | 2P | BG19NUC-HC72 |
| 11117 | 2P | BG19NUC-HC73 |
| 11118 | 2P | BG19NUC-HC74 |
| 11119 | 2P | BG19NUC-HC75 |
| 11120 | RBD | BG19NUC-HC76 |
| 11121 | 2P | BG19NUC-HC77 |
| 11122 | RBD | BG19NUC-HC78 |
| 11123 | 2P | BG19NUC-HC79 |
| 11124 | RBD | BG19NUC-HC80 |
| 11125 | 2P | BG19NUC-HC81 |
| 11126 | 2P | BG19NUC-HC82 |
| 11127 | 2P | BG19NUC-HC83 |
| 11128 | 2P | BG19NUC-HC84 |
| 11129 | 2P | BG19NUC-HC85 |
| 11130 | 2P | BG19NUC-HC86 |
| 11131 | 2P | BG19NUC-HC87 |
| 11132 | 2P | BG19NUC-HC88 |
| 11133 | 2P | BG19NUC-HC89 |
| 11134 | 2P | BG19NUC-HC90 |
| 11135 | 2P | BG19NUC-HC91 |
| 11136 | RBD | BG19NUC-HC92 |
| 11137 | 2P | BG19NUC-HC93 |
| 11138 | 2P | BG19NUC-HC94 |
| 11139 | 2P | BG19NUC-HC95 |
| 11140 | 2P | BG19NUC-HC96 |
| 11141 | 2P | BG19NUC-HC97 |
| 11142 | 2P | BG19NUC-HC98 |
| 11143 | 2P | BG19NUC-HC99 |
| 11144 | 2P | BG19NUC-HC100 |
| 11145 | 2P | BG19NUC-HC101 |
| 11146 | 2P | BG19NUC-HC102 |
| 11147 | 2P | BG19NUC-HC103 |
| 11148 | 2P | BG19NUC-HC104 |
| 11149 | 2P | BG19NUC-HC105 |
| 11150 | RBD | BG19NUC-HC106 |
| 11151 | RBD | BG19NUC-HC107 |
| 11152 | RBD | BG19NUC-HC108 |
| 11153 | 2P | BG19NUC-HC109 |
| 11154 | RBD | BG19NUC-HC110 |
| 11155 | 2P | BG19NUC-HC111 |
| 11156 | 2F | BG19NUC-HC112 |
| 11157 | 2F | BG19NUC-HC113 |
| 11158 | RBD | BG19NUC-HC114 |
| 11159 | 2P | BG19NUC-HC115 |
| 11160 | 2P | BG19NUC-HC116 |
| 11161 | 2P | BG19NUC-HC117 |
| 11162 | RBD | BG19NUC-HC118 |
| 11163 | RBD | BG19NUC-HC119 |
| 11164 | RBD | BG19NUC-HC120 |
| 11165 | RBD | BG19NUC-HC121 |
| 11166 | 2P | BG19NUC-HC122 |
| 11167 | 2P | BG19NUC-HC123 |
| 11168 | 2P | BG19NUC-HC124 |
| 11169 | 2P | BG19NUC-HC125 |
| 11170 | RBD | BG19NUC-HC126 |
| 11171 | RBD | BG19NUC-HC127 |
| 11172 | 2P | BG19NUC-HC128 |
| 11173 | 2P | BG19NUC-HC129 |
| 11174 | 2P | BG19NUC-HC130 |
| 11175 | 2P | BG19NUC-HC131 |
| 11176 | RBD | BG19NUC-HC132 |
| 11177 | 2P | BG19NUC-HC133 |
| 11178 | 2P | BG19NUC-HC134 |
| 11179 | RBD | BG19NUC-HC135 |
| 11180 | RBD | BG19NUC-HC136 |
| 11181 | 2P | BG19NUC-HC137 |
| 11182 | 2P | BG19NUC-HC138 |
| 11183 | RBD | BG19NUC-HC139 |
| 11184 | 2P | BG19NUC-HC140 |
| 11185 | RBD | BG19NUC-HC141 |
| 11186 | 2P | BG19NUC-HC142 |
| 11187 | 2P | BG19NUC-HC143 |
| 11188 | 2P | BG19NUC-HC144 |
| 11189 | 2P | BG19NUC-HC145 |
| 11190 | RBD | BG19NUC-HC146 |
| 11191 | 2P | BG19NUC-HC147 |
| 11192 | 2P | BG19NUC-HC148 |
| 11193 | 2P | BG19NUC-HC149 |
| 11194 | 2P | BG19NUC-HC150 |
| 11195 | 2P | BG19NUC-HC151 |
| 11196 | RBD | BG19NUC-HC152 |
| 11197 | 2P | BG19NUC-HC153 |
| 11198 | 2P | BG19NUC-HC154 |
| 11199 | 2P | BG19NUC-HC155 |
| 11200 | RBD | BG19NUC-HC156 |
| 11201 | 2P | BG19NUC-HC157 |
| 11202 | 2P | BG19NUC-HC158 |
| 11203 | 2P | BG19NUC-HC159 |
| 11204 | 2P | BG19NUC-HC160 |
| 11205 | 2P | BG19NUC-HC161 |
| 11206 | 2P | BG19NUC-HC162 |
| 11207 | 2P | BG19NUC-HC163 |
| 11208 | RBD | BG19NUC-HC164 |
| 11209 | 2P | BG19NUC-HC165 |
| 11210 | 2P | BG19NUC-HC166 |
| 11211 | RBD | BG19NUC-HC167 |
| 11212 | 2P | BG19NUC-HC168 |
| 11213 | 2P | BG19NUC-HC169 |
| 11214 | 2P | BG19NUC-HC170 |
| 11215 | 2P | BG19NUC-HC171 |
| 11216 | 2P | BG19NUC-HC172 |
| 11217 | 2P | BG19NUC-HC173 |
| 11218 | RBD | BG19NUC-HC174 |
| 11219 | RBD | BG19NUC-HC175 |
| 11220 | 2P | BG19NUC-HC176 |
| 11221 | RBD | BG19NUC-HC177 |
| 11222 | RBD | BG19NUC-HC178 |
| 11223 | 2P | BG19NUC-HC179 |
| 11224 | 2P | BG19NUC-HC180 |
| 11225 | 2P | BG19NUC-HC181 |
| 11226 | 2P | BG19NUC-HC182 |
| 11227 | RBD | BG19NUC-HC183 |
| 11228 | RBD | BG19NUC-HC184 |
| 11229 | RBD | BG19NUC-HC185 |
| 11230 | RBD | BG19NUC-HC186 |
| 11231 | RBD | BG19NUC-HC187 |
| 11232 | 2P | BG19NUC-HC188 |
| 11233 | 2P | BG19NUC-HC189 |
| 11234 | RBD | BG19NUC-HC190 |
| 11235 | RBD | BG19NUC-HC191 |
| 11236 | 2P | BG19NUC-HC192 |
| 11237 | 2P | BG19NUC-HC193 |
| 11238 | 2P | BG19NUC-HC194 |
| 11239 | RBD | BG19NUC-HC195 |
| 11240 | RBD | BG19NUC-HC196 |
| 11241 | 2P | BG19NUC-HC197 |
| 11242 | RBD | BG19NUC-HC198 |
| 11243 | 2P | BG19NUC-HC199 |
| 11244 | RBD | BG19NUC-HC200 |
| 11245 | RBD | BG19NUC-HC201 |
| 11246 | RBD | BG19NUC-HC202 |
| 11247 | RBD | BG19NUC-HC203 |
| 11248 | 2P | BG19NUC-HC204 |
| 11249 | 2P | BG19NUC-HC205 |
| 11250 | 2P | BG19NUC-HC206 |
| 11251 | 2P | BG19NUC-HC207 |
| 11252 | 2P | BG19NUC-HC208 |
| 11253 | 2P | BG19NUC-HC209 |
| 11254 | 2P | BG19NUC-HC210 |
| 11255 | 2P | BG19NUC-HC211 |
| 11256 | 2P | BG19NUC-HC212 |
| 11257 | 2P | BG19NUC-HC213 |
| 11258 | RBD | BG19NUC-HC214 |
| 11259 | RBD | BG19NUC-HC215 |
| 11260 | 2P | BG19NUC-HC216 |
| 11261 | 2P | BG19NUC-HC217 |
| 11262 | RBD | BG19NUC-HC218 |
| 11263 | 2P | BG19NUC-HC219 |
| 11264 | 2P | BG19NUC-HC220 |
| 11265 | 2P | BG19NUC-HC221 |
| 11266 | RBD | BG19NUC-HC222 |
| 11267 | RBD | BG19NUC-HC223 |
| 11268 | 2P | BG19NUC-HC224 |
| 11269 | 2P | BG19NUC-HC225 |
| 11270 | 2P | BG19NUC-HC226 |
| 11271 | 2P | BG19NUC-HC227 |
| 11272 | 2P | BG19NUC-HC228 |
| 11273 | RBD | BG19NUC-HC229 |
| 11274 | 2P | BG19NUC-HC230 |
| 11275 | 2P | BG19NUC-HC231 |
| 11276 | RBD | BG19NUC-HC232 |
| 11277 | RBD | BG19NUC-HC233 |
| 11278 | 2P | BG19NUC-HC234 |
| 11279 | 2P | BG19NUC-HC235 |
| 11280 | 2P | BG19NUC-HC236 |
| 11281 | RBD | BG19NUC-HC237 |
| 11282 | RBD | BG19NUC-HC238 |
| 11283 | RBD | BG19NUC-HC239 |
| 11284 | 2P | BG19NUC-HC240 |
| 11285 | 2P | BG19NUC-HC241 |
| 11286 | 2P | BG19NUC-HC242 |
| 11287 | 2P | BG19NUC-HC243 |
| 11288 | 2P | BG19NUC-HC244 |
| 11289 | RBD | BG19NUC-HC245 |
| 11290 | RBD | BG19NUC-HC246 |
| 11291 | 2P | BG19NUC-HC247 |
| 11292 | RBD | BG19NUC-HC248 |
| 11293 | 2P | BG19NUC-HC249 |
| 11294 | 2P | BG19NUC-HC250 |
| 11295 | 2P | BG19NUC-HC251 |
| 11296 | 2P | BG19NUC-HC252 |
| 11297 | 2P | BG19NUC-HC253 |
| 11298 | RBD | BG19NUC-HC254 |
| 11299 | RBD | BG19NUC-HC255 |
| 11300 | 2P | BG19NUC-HC256 |
| 11301 | 2P | BG19NUC-HC257 |
| 11302 | RBD | BG19NUC-HC258 |
| 11303 | 2P | BG19NUC-HC259 |
| 11304 | 2P | BG19NUC-HC260 |
| 11305 | RBD | BG19NUC-HC261 |
| 11306 | 2P | BG19NUC-HC262 |
| 11307 | 2P | BG19NUC-HC263 |
| 11308 | RBD | BG19NUC-HC264 |
| 11309 | 2P | BG19NUC-HC265 |
| 11310 | 2P | BG19NUC-HC266 |
| 11311 | 2P | BG19NUC-HC267 |
| 11312 | 2P | BG19NUC-HC268 |
| 11313 | RBD | BG19NUC-HC269 |
| 11314 | 2P | BG19NUC-HC270 |
| 11315 | RBD | BG19NUC-HC271 |
| 11316 | RBD | BG19NUC-HC272 |
| 11317 | RBD | BG19NUC-HC273 |
| 11318 | 2P | BG19NUC-HC274 |
| 11319 | RBD | BG19NUC-HC275 |
| 11320 | 2P | BG19NUC-LC1 |
| 11321 | RBD | BG19NUC-LC2 |
| 11322 | RBD | BG19NUC-LC3 |
| 11323 | RBD | BG19NUC-LC4 |
| 11324 | 2F | BG19NUC-LC5 |
| 11325 | 2P | BG19NUC-LC6 |
| 11326 | RBD | BG19NUC-LC7 |
| 11327 | RBD | BG19NUC-LC8 |
| 11328 | 2P | BG19NUC-LC9 |
| 11329 | 2P | BG19NUC-LC10 |
| 11330 | 2F | BG19NUC-LC11 |
| 11331 | RBD | BG19NUC-LC12 |
| 11332 | RBD | BG19NUC-LC13 |
| 11333 | 2P | BG19NUC-LC14 |
| 11334 | RBD | BG19NUC-LC15 |
| 11335 | RBD | BG19NUC-LC16 |
| 11336 | 2P | BG19NUC-LC17 |
| 11337 | 2P | BG19NUC-LC18 |
| 11338 | 2P | BG19NUC-LC19 |
| 11339 | 2F | BG19NUC-LC20 |
| 11340 | 2F | BG19NUC-LC21 |
| 11341 | 2P | BG19NUC-LC22 |
| 11342 | RBD | BG19NUC-LC23 |
| 11343 | 2P | BG19NUC-LC24 |
| 11344 | 2P | BG19NUC-LC25 |
| 11345 | RBD | BG19NUC-LC26 |
| 11346 | 2P | BG19NUC-LC27 |
| 11347 | RBD | BG19NUC-LC28 |
| 11348 | 2P | BG19NUC-LC29 |
| 11349 | RBD | BG19NUC-LC30 |
| 11350 | 2P | BG19NUC-LC31 |
| 11351 | 2P | BG19NUC-LC32 |
| 11352 | 2P | BG19NUC-LC33 |
| 11353 | RBD | BG19NUC-LC34 |
| 11354 | 2P | BG19NUC-LC35 |
| 11355 | RBD | BG19NUC-LC36 |
| 11356 | RBD | BG19NUC-LC37 |
| 11357 | 2P | BG19NUC-LC38 |
| 11358 | 2P | BG19NUC-LC39 |
| 11359 | RBD | BG19NUC-LC40 |
| 11360 | 2P | BG19NUC-LC41 |
| 11361 | 2P | BG19NUC-LC42 |
| 11362 | 2P | BG19NUC-LC43 |
| 11363 | 2P | BG19NUC-LC44 |
| 11364 | 2P | BG19NUC-LC45 |
| 11365 | 2P | BG19NUC-LC46 |
| 11366 | RBD | BG19NUC-LC47 |
| 11367 | RBD | BG19NUC-LC48 |
| 11368 | RBD | BG19NUC-LC49 |
| 11369 | 2P | BG19NUC-LC50 |
| 11370 | 2P | BG19NUC-LC51 |
| 11371 | 2P | BG19NUC-LC52 |
| 11372 | 2P | BG19NUC-LC53 |
| 11373 | RBD | BG19NUC-LC54 |
| 11374 | 2P | BG19NUC-LC55 |
| 11375 | 2P | BG19NUC-LC56 |
| 11376 | 2P | BG19NUC-LC57 |
| 11377 | 2P | BG19NUC-LC58 |
| 11378 | RBD | BG19NUC-LC59 |
| 11379 | RBD | BG19NUC-LC60 |
| 11380 | RBD | BG19NUC-LC61 |
| 11381 | 2P | BG19NUC-LC62 |
| 11382 | 2P | BG19NUC-LC63 |
| 11383 | RBD | BG19NUC-LC64 |
| 11384 | 2P | BG19NUC-LC65 |
| 11385 | 2P | BG19NUC-LC66 |
| 11386 | 2P | BG19NUC-LC67 |
| 11387 | 2P | BG19NUC-LC68 |
| 11388 | 2P | BG19NUC-LC69 |
| 11389 | 2P | BG19NUC-LC70 |
| 11390 | 2P | BG19NUC-LC71 |
| 11391 | 2P | BG19NUC-LC72 |
| 11392 | 2P | BG19NUC-LC73 |
| 11393 | 2P | BG19NUC-LC74 |
| 11394 | 2P | BG19NUC-LC75 |
| 11395 | RBD | BG19NUC-LC76 |
| 11396 | 2P | BG19NUC-LC77 |
| 11397 | RBD | BG19NUC-LC78 |
| 11398 | 2P | BG19NUC-LC79 |
| 11399 | RBD | BG19NUC-LC80 |
| 11400 | 2P | BG19NUC-LC81 |
| 11401 | 2P | BG19NUC-LC82 |
| 11402 | 2P | BG19NUC-LC83 |
| 11403 | 2P | BG19NUC-LC84 |
| 11404 | 2P | BG19NUC-LC85 |
| 11405 | 2P | BG19NUC-LC86 |
| 11406 | 2P | BG19NUC-LC87 |
| 11407 | 2P | BG19NUC-LC88 |
| 11408 | 2P | BG19NUC-LC89 |
| 11409 | 2P | BG19NUC-LC90 |
| 11410 | 2P | BG19NUC-LC91 |
| 11411 | RBD | BG19NUC-LC92 |
| 11412 | 2P | BG19NUC-LC93 |
| 11413 | 2P | BG19NUC-LC94 |
| 11414 | 2P | BG19NUC-LC95 |
| 11415 | 2P | BG19NUC-LC96 |
| 11416 | 2P | BG19NUC-LC97 |
| 11417 | 2P | BG19NUC-LC98 |
| 11418 | 2P | BG19NUC-LC99 |
| 11419 | 2P | BG19NUC-LC100 |
| 11420 | 2P | BG19NUC-LC101 |
| 11421 | 2P | BG19NUC-LC102 |
| 11422 | 2P | BG19NUC-LC103 |
| 11423 | 2P | BG19NUC-LC104 |
| 11424 | 2F | BG19NUC-LC105 |
| 11425 | RBD | BG19NUC-LC106 |
| 11426 | RBD | BG19NUC-LC107 |
| 11427 | RBD | BG19NUC-LC108 |
| 11428 | 2P | BG19NUC-LC109 |
| 11429 | RBD | BG19NUC-LC110 |
| 11430 | 2P | BG19NUC-LC111 |
| 11431 | 2P | BG19NUC-LC112 |
| 11432 | 2P | BG19NUC-LC113 |
| 11433 | RBD | BG19NUC-LC114 |
| 11434 | 2P | BG19NUC-LC115 |
| 11435 | 2P | BG19NUC-LC116 |
| 11436 | 2P | BG19NUC-LC117 |
| 11437 | RBD | BG19NUC-LC118 |
| 11438 | RBD | BG19NUC-LC119 |
| 11439 | RBD | BG19NUC-LC120 |
| 11440 | RBD | BG19NUC-LC121 |
| 11441 | 2P | BG19NUC-LC122 |
| 11442 | 2P | BG19NUC-LC123 |
| 11443 | 2P | BG19NUC-LC124 |
| 11444 | 2P | BG19NUC-LC125 |
| 11445 | RBD | BG19NUC-LC126 |
| 11446 | RBD | BG19NUC-LC127 |
| 11447 | 2P | BG19NUC-LC128 |
| 11448 | 2P | BG19NUC-LC129 |
| 11449 | 2P | BG19NUC-LC130 |
| 11450 | 2P | BG19NUC-LC131 |
| 11451 | RBD | BG19NUC-LC132 |
| 11452 | 2P | BG19NUC-LC133 |
| 11453 | 2P | BG19NUC-LC134 |
| 11454 | RBD | BG19NUC-LC135 |
| 11455 | RBD | BG19NUC-LC136 |
| 11456 | 2P | BG19NUC-LC137 |
| 11457 | 2P | BG19NUC-LC138 |
| 11458 | RBD | BG19NUC-LC139 |
| 11459 | 2P | BG19NUC-LC140 |
| 11460 | RBD | BG19NUC-LC141 |
| 11461 | 2P | BG19NUC-LC142 |
| 11462 | 2P | BG19NUC-LC143 |
| 11463 | 2P | BG19NUC-LC144 |
| 11464 | 2P | BG19NUC-LC145 |
| 11465 | RBD | BG19NUC-LC146 |
| 11466 | 2P | BG19NUC-LC147 |
| 11467 | 2P | BG19NUC-LC148 |
| 11468 | 2P | BG19NUC-LC149 |
| 11469 | 2P | BG19NUC-LC150 |
| 11470 | 2P | BG19NUC-LC151 |
| 11471 | RBD | BG19NUC-LC152 |
| 11472 | 2P | BG19NUC-LC153 |
| 11473 | 2P | BG19NUC-LC154 |
| 11474 | 2P | BG19NUC-LC155 |
| 11475 | RBD | BG19NUC-LC156 |
| 11476 | 2P | BG19NUC-LC157 |
| 11477 | 2P | BG19NUC-LC158 |
| 11478 | 2P | BG19NUC-LC159 |
| 11479 | 2P | BG19NUC-LC160 |
| 11480 | 2P | BG19NUC-LC161 |
| 11481 | 2P | BG19NUC-LC162 |
| 11482 | 2P | BG19NUC-LC163 |
| 11483 | RBD | BG19NUC-LC164 |
| 11484 | 2P | BG19NUC-LC165 |
| 11485 | 2P | BG19NUC-LC166 |
| 11486 | RBD | BG19NUC-LC167 |
| 11487 | 2P | BG19NUC-LC168 |
| 11488 | 2P | BG19NUC-LC169 |
| 11489 | 2P | BG19NUC-LC170 |
| 11490 | 2P | BG19NUC-LC171 |
| 11491 | 2P | BG19NUC-LC172 |
| 11492 | 2P | BG19NUC-LC173 |
| 11493 | RBD | BG19NUC-LC174 |
| 11494 | RBD | BG19NUC-LC175 |
| 11495 | 2P | BG19NUC-LC176 |
| 11496 | RBD | BG19NUC-LC177 |
| 11497 | RBD | BG19NUC-LC178 |
| 11498 | 2P | BG19NUC-LC179 |
| 11499 | 2P | BG19NUC-LC180 |
| 11500 | 2P | BG19NUC-LC181 |
| 11501 | 2P | BG19NUC-LC182 |
| 11502 | RBD | BG19NUC-LC183 |
| 11503 | RBD | BG19NUC-LC184 |
| 11504 | RBD | BG19NUC-LC185 |
| 11505 | RBD | BG19NUC-LC186 |
| 11506 | RBD | BG19NUC-LC187 |
| 11507 | 2P | BG19NUC-LC188 |
| 11508 | 2P | BG19NUC-LC189 |
| 11509 | RBD | BG19NUC-LC190 |
| 11510 | RBD | BG19NUC-LC191 |
| 11511 | 2P | BG19NUC-LC192 |
| 11512 | 2P | BG19NUC-LC193 |
| 11513 | 2P | BG19NUC-LC194 |
| 11514 | RBD | BG19NUC-LC195 |
| 11515 | RBD | BG19NUC-LC196 |
| 11516 | 2P | BG19NUC-LC197 |
| 11517 | RBD | BG19NUC-LC198 |
| 11518 | 2P | BG19NUC-LC199 |
| 11519 | RBD | BG19NUC-LC200 |
| 11520 | RBD | BG19NUC-LC201 |
| 11521 | RBD | BG19NUC-LC202 |
| 11522 | RBD | BG19NUC-LC203 |
| 11523 | 2P | BG19NUC-LC204 |
| 11524 | 2P | BG19NUC-LC205 |
| 11525 | 2P | BG19NUC-LC206 |
| 11526 | 2P | BG19NUC-LC207 |
| 11527 | 2P | BG19NUC-LC208 |
| 11528 | 2P | BG19NUC-LC209 |
| 11529 | 2P | BG19NUC-LC210 |
| 11530 | 2P | BG19NUC-LC211 |
| 11531 | 2P | BG19NUC-LC212 |
| 11532 | 2P | BG19NUC-LC213 |
| 11533 | RBD | BG19NUC-LC214 |
| 11534 | RBD | BG19NUC-LC215 |
| 11535 | 2P | BG19NUC-LC216 |
| 11536 | 2P | BG19NUC-LC217 |
| 11537 | RBD | BG19NUC-LC218 |
| 11538 | 2P | BG19NUC-LC219 |
| 11539 | 2P | BG19NUC-LC220 |
| 11540 | 2P | BG19NUC-LC221 |
| 11541 | RBD | BG19NUC-LC222 |
| 11542 | RBD | BG19NUC-LC223 |
| 11543 | 2P | BG19NUC-LC224 |
| 11544 | 2P | BG19NUC-LC225 |
| 11545 | 2P | BG19NUC-LC226 |
| 11546 | 2P | BG19NUC-LC227 |
| 11547 | 2P | BG19NUC-LC228 |
| 11548 | RBD | BG19NUC-LC229 |
| 11549 | 2P | BG19NUC-LC230 |
| 11550 | 2P | BG19NUC-LC231 |
| 11551 | RBD | BG19NUC-LC232 |
| 11552 | RBD | BG19NUC-LC233 |
| 11553 | 2P | BG19NUC-LC234 |
| 11554 | 2P | BG19NUC-LC235 |
| 11555 | 2P | BG19NUC-LC236 |
| 11556 | RBD | BG19NUC-LC237 |
| 11557 | RBD | BG19NUC-LC238 |
| 11558 | RBD | BG19NUC-LC239 |
| 11559 | 2P | BG19NUC-LC240 |
| 11560 | 2P | BG19NUC-LC241 |
| 11561 | 2P | BG19NUC-LC242 |
| 11562 | 2P | BG19NUC-LC243 |
| 11563 | 2P | BG19NUC-LC244 |
| 11564 | RBD | BG19NUC-LC245 |
| 11565 | RBD | BG19NUC-LC246 |
| 11566 | 2P | BG19NUC-LC247 |
| 11567 | RBD | BG19NUC-LC248 |
| 11568 | 2P | BG19NUC-LC249 |
| 11569 | 2P | BG19NUC-LC250 |
| 11570 | 2P | BG19NUC-LC251 |
| 11571 | 2P | BG19NUC-LC252 |
| 11572 | 2P | BG19NUC-LC253 |
| 11573 | RBD | BG19NUC-LC254 |
| 11574 | RBD | BG19NUC-LC255 |
| 11575 | 2P | BG19NUC-LC256 |
| 11576 | 2P | BG19NUC-LC257 |
| 11577 | RBD | BG19NUC-LC258 |
| 11578 | 2P | BG19NUC-LC259 |
| 11579 | 2P | BG19NUC-LC260 |
| 11580 | RBD | BG19NUC-LC261 |
| 11581 | 2P | BG19NUC-LC262 |
| 11582 | 2P | BG19NUC-LC263 |
| 11583 | RBD | BG19NUC-LC264 |
| 11584 | 2P | BG19NUC-LC265 |
| 11585 | 2P | BG19NUC-LC266 |
| 11586 | 2P | BG19NUC-LC267 |
| 11587 | 2P | BG19NUC-LC268 |
| 11588 | RBD | BG19NUC-LC269 |
| 11589 | 2P | BG19NUC-LC270 |
| 11590 | RBD | BG19NUC-LC271 |
| 11591 | RBD | BG19NUC-LC272 |
| 11592 | RBD | BG19NUC-LC273 |
| 11593 | 2P | BG19NUC-LC274 |
| 11594 | RBD | BG19NUC-LC275 |
| 11595 | 2P | BG20NUC-HC1 |
| 11596 | 2P | BG20NUC-HC2 |
| 11597 | 2P | BG20NUC-HC3 |
| 11598 | RBD | BG20NUC-HC4 |
| 11599 | 2P | BG20NUC-HC5 |
| 11600 | 2P | BG20NUC-HC6 |
| 11601 | 2P | BG20NUC-HC7 |
| 11602 | 2P | BG20NUC-HC8 |
| 11603 | 2P | BG20NUC-HC9 |
| 11604 | 2P | BG20NUC-HC10 |
| 11605 | 2P | BG20NUC-HC11 |
| 11606 | 2P | BG20NUC-HC12 |
| 11607 | RBD | BG20NUC-HC13 |
| 11608 | 2P | BG20NUC-HC14 |
| 11609 | 2P | BG20NUC-HC15 |
| 11610 | 2P | BG20NUC-HC16 |
| 11611 | 2P | BG20NUC-HC17 |
| 11612 | RBD | BG20NUC-HC18 |
| 11613 | RBD | BG20NUC-HC19 |
| 11614 | RBD | BG20NUC-HC20 |
| 11615 | 2P | BG20NUC-HC21 |
| 11616 | 2P | BG20NUC-HC22 |
| 11617 | 2P | BG20NUC-HC23 |
| 11618 | 2F | BG20NUC-HC24 |
| 11619 | 2P | BG20NUC-HC25 |
| 11620 | 2F | BG20NUC-HC26 |
| 11621 | 2P | BG20NUC-HC27 |
| 11622 | 2F | BG20NUC-HC28 |
| 11623 | 2P | BG20NUC-HC29 |
| 11624 | 2P | BG20NUC-HC30 |
| 11625 | RBD | BG20NUC-HC31 |
| 11626 | 2P | BG20NUC-HC32 |
| 11627 | 2P | BG20NUC-HC33 |
| 11628 | RBD | BG20NUC-HC34 |
| 11629 | 2P | BG20NUC-HC35 |
| 11630 | 2P | BG20NUC-HC36 |
| 11631 | 2P | BG20NUC-HC37 |
| 11632 | 2P | BG20NUC-HC38 |
| 11633 | RBD | BG20NUC-HC39 |
| 11634 | 2P | BG20NUC-HC40 |
| 11635 | 2P | BG20NUC-HC41 |
| 11636 | 2P | BG20NUC-HC42 |
| 11637 | 2P | BG20NUC-HC43 |
| 11638 | 2P | BG20NUC-HC44 |
| 11639 | RBD | BG20NUC-HC45 |
| 11640 | 2P | BG20NUC-HC46 |
| 11641 | 2P | BG20NUC-HC47 |
| 11642 | RBD | BG20NUC-HC48 |
| 11643 | RBD | BG20NUC-HC49 |
| 11644 | 2P | BG20NUC-HC50 |
| 11645 | 2P | BG20NUC-HC51 |
| 11646 | 2P | BG20NUC-HC52 |
| 11647 | RBD | BG20NUC-HC53 |
| 11648 | 2P | BG20NUC-HC54 |
| 11649 | 2P | BG20NUC-HC55 |
| 11650 | 2P | BG20NUC-HC56 |
| 11651 | RBD | BG20NUC-HC57 |
| 11652 | 2P | BG20NUC-HC58 |
| 11653 | 2F | BG20NUC-HC59 |
| 11654 | 2P | BG20NUC-HC60 |
| 11655 | 2P | BG20NUC-HC61 |
| 11656 | 2P | BG20NUC-HC62 |
| 11657 | RBD | BG20NUC-HC63 |
| 11658 | 2P | BG20NUC-HC64 |
| 11659 | 2P | BG20NUC-HC65 |
| 11660 | 2P | BG20NUC-HC66 |
| 11661 | 2P | BG20NUC-HC67 |
| 11662 | 2P | BG20NUC-HC68 |
| 11663 | 2P | BG20NUC-HC69 |
| 11664 | 2P | BG20NUC-HC70 |
| 11665 | RBD | BG20NUC-HC71 |
| 11666 | 2P | BG20NUC-HC72 |
| 11667 | 2P | BG20NUC-HC73 |
| 11668 | 2P | BG20NUC-HC74 |
| 11669 | 2P | BG20NUC-HC75 |
| 11670 | 2P | BG20NUC-HC76 |
| 11671 | 2P | BG20NUC-HC77 |
| 11672 | 2P | BG20NUC-HC78 |
| 11673 | 2P | BG20NUC-HC79 |
| 11674 | 2P | BG20NUC-HC80 |
| 11675 | 2P | BG20NUC-HC81 |
| 11676 | 2P | BG20NUC-HC82 |
| 11677 | 2P | BG20NUC-HC83 |
| 11678 | 2P | BG20NUC-HC84 |
| 11679 | RBD | BG20NUC-HC85 |
| 11680 | RBD | BG20NUC-HC86 |
| 11681 | 2P | BG20NUC-HC87 |
| 11682 | RBD | BG20NUC-HC88 |
| 11683 | RBD | BG20NUC-HC89 |
| 11684 | 2P | BG20NUC-HC90 |
| 11685 | RBD | BG20NUC-HC91 |
| 11686 | RBD | BG20NUC-HC92 |
| 11687 | 2P | BG20NUC-HC93 |
| 11688 | 2P | BG20NUC-HC94 |
| 11689 | 2P | BG20NUC-HC95 |
| 11690 | 2P | BG20NUC-HC96 |
| 11691 | 2P | BG20NUC-HC97 |
| 11692 | 2P | BG20NUC-HC98 |
| 11693 | 2P | BG20NUC-HC99 |
| 11694 | 2P | BG20NUC-HC100 |
| 11695 | 2F | BG20NUC-HC101 |
| 11696 | RBD | BG20NUC-HC102 |
| 11697 | 2P | BG20NUC-HC103 |
| 11698 | 2P | BG20NUC-HC104 |
| 11699 | RBD | BG20NUC-HC105 |
| 11700 | RBD | BG20NUC-HC106 |
| 11701 | RBD | BG20NUC-HC107 |
| 11702 | 2P | BG20NUC-HC108 |
| 11703 | 2P | BG20NUC-HC109 |
| 11704 | 2P | BG20NUC-HC110 |
| 11705 | 2P | BG20NUC-HC111 |
| 11706 | 2F | BG20NUC-HC112 |
| 11707 | 2P | BG20NUC-HC113 |
| 11708 | RBD | BG20NUC-HC114 |
| 11709 | 2P | BG20NUC-HC115 |
| 11710 | 2P | BG20NUC-HC116 |
| 11711 | 2P | BG20NUC-HC117 |
| 11712 | RBD | BG20NUC-HC118 |
| 11713 | 2P | BG20NUC-HC119 |
| 11714 | 2P | BG20NUC-HC120 |
| 11715 | 2P | BG20NUC-HC121 |
| 11716 | 2P | BG20NUC-HC122 |
| 11717 | 2P | BG20NUC-HC123 |
| 11718 | 2P | BG20NUC-HC124 |
| 11719 | 2F | BG20NUC-HC125 |
| 11720 | RBD | BG20NUC-HC126 |
| 11721 | 2P | BG20NUC-HC127 |
| 11722 | 2P | BG20NUC-HC128 |
| 11723 | 2P | BG20NUC-HC129 |
| 11724 | RBD | BG20NUC-HC130 |
| 11725 | RBD | BG20NUC-HC131 |
| 11726 | RBD | BG20NUC-HC132 |
| 11727 | 2P | BG20NUC-HC133 |
| 11728 | RBD | BG20NUC-HC134 |
| 11729 | 2P | BG20NUC-HC135 |
| 11730 | 2P | BG20NUC-HC136 |
| 11731 | RBD | BG20NUC-HC137 |
| 11732 | RBD | BG20NUC-HC138 |
| 11733 | RBD | BG20NUC-HC139 |
| 11734 | 2P | BG20NUC-HC140 |
| 11735 | RBD | BG20NUC-HC141 |
| 11736 | 2P | BG20NUC-HC142 |
| 11737 | 2P | BG20NUC-HC143 |
| 11738 | 2P | BG20NUC-HC144 |
| 11739 | 2P | BG20NUC-HC145 |
| 11740 | 2P | BG20NUC-HC146 |
| 11741 | 2P | BG20NUC-HC147 |
| 11742 | RBD | BG20NUC-HC148 |
| 11743 | 2P | BG20NUC-HC149 |
| 11744 | 2F | BG20NUC-HC150 |
| 11745 | RBD | BG20NUC-HC151 |
| 11746 | 2P | BG20NUC-HC152 |
| 11747 | RBD | BG20NUC-HC153 |
| 11748 | RBD | BG20NUC-HC154 |
| 11749 | 2P | BG20NUC-HC155 |
| 11750 | RBD | BG20NUC-HC156 |
| 11751 | RBD | BG20NUC-HC157 |
| 11752 | RBD | BG20NUC-HC158 |
| 11753 | 2P | BG20NUC-HC159 |
| 11754 | 2P | BG20NUC-HC160 |
| 11755 | 2P | BG20NUC-HC161 |
| 11756 | 2P | BG20NUC-HC162 |
| 11757 | RBD | BG20NUC-HC163 |
| 11758 | 2P | BG20NUC-HC164 |
| 11759 | RBD | BG20NUC-HC165 |
| 11760 | 2P | BG20NUC-HC166 |
| 11761 | 2P | BG20NUC-HC167 |
| 11762 | 2P | BG20NUC-HC168 |
| 11763 | 2P | BG20NUC-HC169 |
| 11764 | 2P | BG20NUC-HC170 |
| 11765 | 2P | BG20NUC-HC171 |
| 11766 | RBD | BG20NUC-HC172 |
| 11767 | 2P | BG20NUC-HC173 |
| 11768 | RBD | BG20NUC-HC174 |
| 11769 | 2P | BG20NUC-HC175 |
| 11770 | 2P | BG20NUC-HC176 |
| 11771 | 2P | BG20NUC-LC1 |
| 11772 | 2F | BG20NUC-LC2 |
| 11773 | 2P | BG20NUC-LC3 |
| 11774 | RBD | BG20NUC-LC4 |
| 11775 | 2P | BG20NUC-LC5 |
| 11776 | 2P | BG20NUC-LC6 |
| 11777 | 2P | BG20NUC-LC7 |
| 11778 | 2P | BG20NUC-LC8 |
| 11779 | 2P | BG20NUC-LC9 |
| 11780 | 2P | BG20NUC-LC10 |
| 11781 | 2F | BG20NUC-LC11 |
| 11782 | 2F | BG20NUC-LC12 |
| 11783 | RBD | BG20NUC-LC13 |
| 11784 | 2P | BG20NUC-LC14 |
| 11785 | 2P | BG20NUC-LC15 |
| 11786 | 2P | BG20NUC-LC16 |
| 11787 | 2P | BG20NUC-LC17 |
| 11788 | RBD | BG20NUC-LC18 |
| 11789 | RBD | BG20NUC-LC19 |
| 11790 | RBD | BG20NUC-LC20 |
| 11791 | 2P | BG20NUC-LC21 |
| 11792 | 2F | BG20NUC-LC22 |
| 11793 | 2F | BG20NUC-LC23 |
| 11794 | 2P | BG20NUC-LC24 |
| 11795 | 2P | BG20NUC-LC25 |
| 11796 | 2P | BG20NUC-LC26 |
| 11797 | 2P | BG20NUC-LC27 |
| 11798 | 2P | BG20NUC-LC28 |
| 11799 | 2P | BG20NUC-LC29 |
| 11800 | 2P | BG20NUC-LC30 |
| 11801 | RBD | BG20NUC-LC31 |
| 11802 | 2P | BG20NUC-LC32 |
| 11803 | 2F | BG20NUC-LC33 |
| 11804 | RBD | BG20NUC-LC34 |
| 11805 | 2P | BG20NUC-LC35 |
| 11806 | 2P | BG20NUC-LC36 |
| 11807 | 2P | BG20NUC-LC37 |
| 11808 | 2P | BG20NUC-LC38 |
| 11809 | RBD | BG20NUC-LC39 |
| 11810 | 2P | BG20NUC-LC40 |
| 11811 | 2P | BG20NUC-LC41 |
| 11812 | 2P | BG20NUC-LC42 |
| 11813 | 2P | BG20NUC-LC43 |
| 11814 | 2F | BG20NUC-LC44 |
| 11815 | RBD | BG20NUC-LC45 |
| 11816 | 2P | BG20NUC-LC46 |
| 11817 | 2P | BG20NUC-LC47 |
| 11818 | RBD | BG20NUC-LC48 |
| 11819 | RBD | BG20NUC-LC49 |
| 11820 | 2P | BG20NUC-LC50 |
| 11821 | 2P | BG20NUC-LC51 |
| 11822 | 2P | BG20NUC-LC52 |
| 11823 | RBD | BG20NUC-LC53 |
| 11824 | 2P | BG20NUC-LC54 |
| 11825 | 2P | BG20NUC-LC55 |
| 11826 | 2P | BG20NUC-LC56 |
| 11827 | RBD | BG20NUC-LC57 |
| 11828 | 2P | BG20NUC-LC58 |
| 11829 | 2P | BG20NUC-LC59 |
| 11830 | 2P | BG20NUC-LC60 |
| 11831 | 2P | BG20NUC-LC61 |
| 11832 | 2P | BG20NUC-LC62 |
| 11833 | RBD | BG20NUC-LC63 |
| 11834 | 2P | BG20NUC-LC64 |
| 11835 | 2P | BG20NUC-LC65 |
| 11836 | 2P | BG20NUC-LC66 |
| 11837 | 2P | BG20NUC-LC67 |
| 11838 | 2P | BG20NUC-LC68 |
| 11839 | 2P | BG20NUC-LC69 |
| 11840 | 2F | BG20NUC-LC70 |
| 11841 | RBD | BG20NUC-LC71 |
| 11842 | 2P | BG20NUC-LC72 |
| 11843 | 2P | BG20NUC-LC73 |
| 11844 | 2P | BG20NUC-LC74 |
| 11845 | 2P | BG20NUC-LC75 |
| 11846 | 2P | BG20NUC-LC76 |
| 11847 | 2P | BG20NUC-LC77 |
| 11848 | 2P | BG20NUC-LC78 |
| 11849 | 2P | BG20NUC-LC79 |
| 11850 | 2P | BG20NUC-LC80 |
| 11851 | 2P | BG20NUC-LC81 |
| 11852 | 2P | BG20NUC-LC82 |
| 11853 | 2P | BG20NUC-LC83 |
| 11854 | 2P | BG20NUC-LC84 |
| 11855 | RBD | BG20NUC-LC85 |
| 11856 | RBD | BG20NUC-LC86 |
| 11857 | 2P | BG20NUC-LC87 |
| 11858 | RBD | BG20NUC-LC88 |
| 11859 | RBD | BG20NUC-LC89 |
| 11860 | 2P | BG20NUC-LC90 |
| 11861 | RBD | BG20NUC-LC91 |
| 11862 | RBD | BG20NUC-LC92 |
| 11863 | 2P | BG20NUC-LC93 |
| 11864 | 2P | BG20NUC-LC94 |
| 11865 | 2P | BG20NUC-LC95 |
| 11866 | 2P | BG20NUC-LC96 |
| 11867 | 2P | BG20NUC-LC97 |
| 11868 | 2P | BG20NUC-LC98 |
| 11869 | 2P | BG20NUC-LC99 |
| 11870 | 2P | BG20NUC-LC100 |
| 11871 | 2P | BG20NUC-LC101 |
| 11872 | RBD | BG20NUC-LC102 |
| 11873 | 2P | BG20NUC-LC103 |
| 11874 | 2F | BG20NUC-LC104 |
| 11875 | RBD | BG20NUC-LC105 |
| 11876 | RBD | BG20NUC-LC106 |
| 11877 | RBD | BG20NUC-LC107 |
| 11878 | 2P | BG20NUC-LC108 |
| 11879 | 2P | BG20NUC-LC109 |
| 11880 | 2P | BG20NUC-LC110 |
| 11881 | 2P | BG20NUC-LC111 |
| 11882 | 2P | BG20NUC-LC112 |
| 11883 | 2P | BG20NUC-LC113 |
| 11884 | RBD | BG20NUC-LC114 |
| 11885 | 2P | BG20NUC-LC115 |
| 11886 | 2P | BG20NUC-LC116 |
| 11887 | 2P | BG20NUC-LC117 |
| 11888 | RBD | BG20NUC-LC118 |
| 11889 | 2P | BG20NUC-LC119 |
| 11890 | 2P | BG20NUC-LC120 |
| 11891 | 2P | BG20NUC-LC121 |
| 11892 | 2P | BG20NUC-LC122 |
| 11893 | 2P | BG20NUC-LC123 |
| 11894 | 2P | BG20NUC-LC124 |
| 11895 | 2P | BG20NUC-LC125 |
| 11896 | RBD | BG20NUC-LC126 |
| 11897 | 2P | BG20NUC-LC127 |
| 11898 | 2P | BG20NUC-LC128 |
| 11899 | 2P | BG20NUC-LC129 |
| 11900 | RBD | BG20NUC-LC130 |
| 11901 | RBD | BG20NUC-LC131 |
| 11902 | RBD | BG20NUC-LC132 |
| 11903 | 2P | BG20NUC-LC133 |
| 11904 | RBD | BG20NUC-LC134 |
| 11905 | 2P | BG20NUC-LC135 |
| 11906 | 2P | BG20NUC-LC136 |
| 11907 | RBD | BG20NUC-LC137 |
| 11908 | RBD | BG20NUC-LC138 |
| 11909 | RBD | BG20NUC-LC139 |
| 11910 | 2P | BG20NUC-LC140 |
| 11911 | RBD | BG20NUC-LC141 |
| 11912 | 2P | BG20NUC-LC142 |
| 11913 | 2P | BG20NUC-LC143 |
| 11914 | 2P | BG20NUC-LC144 |
| 11915 | 2P | BG20NUC-LC145 |
| 11916 | 2P | BG20NUC-LC146 |
| 11917 | 2P | BG20NUC-LC147 |
| 11918 | RBD | BG20NUC-LC148 |
| 11919 | 2P | BG20NUC-LC149 |
| 11920 | 2P | BG20NUC-LC150 |
| 11921 | RBD | BG20NUC-LC151 |
| 11922 | 2P | BG20NUC-LC152 |
| 11923 | RBD | BG20NUC-LC153 |
| 11924 | RBD | BG20NUC-LC154 |
| 11925 | 2P | BG20NUC-LC155 |
| 11926 | RBD | BG20NUC-LC156 |
| 11927 | RBD | BG20NUC-LC157 |
| 11928 | RBD | BG20NUC-LC158 |
| 11929 | 2P | BG20NUC-LC159 |
| 11930 | 2P | BG20NUC-LC160 |
| 11931 | 2P | BG20NUC-LC161 |
| 11932 | 2P | BG20NUC-LC162 |
| 11933 | RBD | BG20NUC-LC163 |
| 11934 | 2P | BG20NUC-LC164 |
| 11935 | RBD | BG20NUC-LC165 |
| 11936 | 2P | BG20NUC-LC166 |
| 11937 | 2P | BG20NUC-LC167 |
| 11938 | 2P | BG20NUC-LC168 |
| 11939 | 2P | BG20NUC-LC169 |
| 11940 | 2P | BG20NUC-LC170 |
| 11941 | 2P | BG20NUC-LC171 |
| 11942 | RBD | BG20NUC-LC172 |
| 11943 | 2P | BG20NUC-LC173 |
| 11944 | RBD | BG20NUC-LC174 |
| 11945 | 2P | BG20NUC-LC175 |
| 11946 | 2P | BG20NUC-LC176 |
| 11947 | 2P | BG21NUC-HC1 |
| 11948 | 2P | BG21NUC-HC2 |
| 11949 | RBD | BG21NUC-HC3 |
| 11950 | 2P | BG21NUC-HC4 |
| 11951 | 2P | BG21NUC-HC5 |
| 11952 | 2P | BG21NUC-HC6 |
| 11953 | 2P | BG21NUC-HC7 |
| 11954 | 2P | BG21NUC-HC8 |
| 11955 | 2P | BG21NUC-HC9 |
| 11956 | 2P | BG21NUC-HC10 |
| 11957 | 2P | BG21NUC-HC11 |
| 11958 | RBD | BG21NUC-HC12 |
| 11959 | 2P | BG21NUC-HC13 |
| 11960 | 2P | BG21NUC-HC14 |
| 11961 | 2P | BG21NUC-HC15 |
| 11962 | 2P | BG21NUC-HC16 |
| 11963 | 2P | BG21NUC-HC17 |
| 11964 | 2P | BG21NUC-HC18 |
| 11965 | 2P | BG21NUC-HC19 |
| 11966 | 2P | BG21NUC-HC20 |
| 11967 | 2P | BG21NUC-HC21 |
| 11968 | RBD | BG21NUC-HC22 |
| 11969 | 2P | BG21NUC-HC23 |
| 11970 | 2P | BG21NUC-HC24 |
| 11971 | 2P | BG21NUC-HC25 |
| 11972 | 2P | BG21NUC-HC26 |
| 11973 | RBD | BG21NUC-HC27 |
| 11974 | 2P | BG21NUC-HC28 |
| 11975 | 2P | BG21NUC-HC29 |
| 11976 | 2P | BG21NUC-HC30 |
| 11977 | 2P | BG21NUC-HC31 |
| 11978 | 2P | BG21NUC-HC32 |
| 11979 | RBD | BG21NUC-HC33 |
| 11980 | 2P | BG21NUC-HC34 |
| 11981 | 2P | BG21NUC-HC35 |
| 11982 | RBD | BG21NUC-HC36 |
| 11983 | 2P | BG21NUC-HC37 |
| 11984 | 2P | BG21NUC-HC38 |
| 11985 | 2P | BG21NUC-HC39 |
| 11986 | 2P | BG21NUC-HC40 |
| 11987 | 2P | BG21NUC-HC41 |
| 11988 | RBD | BG21NUC-HC42 |
| 11989 | 2P | BG21NUC-HC43 |
| 11990 | 2P | BG21NUC-HC44 |
| 11991 | RBD | BG21NUC-HC45 |
| 11992 | 2P | BG21NUC-HC46 |
| 11993 | 2P | BG21NUC-HC47 |
| 11994 | 2P | BG21NUC-HC48 |
| 11995 | 2F | BG21NUC-HC49 |
| 11996 | 2P | BG21NUC-HC50 |
| 11997 | RBD | BG21NUC-HC51 |
| 11998 | RBD | BG21NUC-HC52 |
| 11999 | 2P | BG21NUC-HC53 |
| 12000 | RBD | BG21NUC-HC54 |
| 12001 | 2F | BG21NUC-HC55 |
| 12002 | RBD | BG21NUC-HC56 |
| 12003 | 2P | BG21NUC-HC57 |
| 12004 | RBD | BG21NUC-HC58 |
| 12005 | RBD | BG21NUC-HC59 |
| 12006 | RBD | BG21NUC-HC60 |
| 12007 | RBD | BG21NUC-HC61 |
| 12008 | RBD | BG21NUC-HC62 |
| 12009 | RBD | BG21NUC-HC63 |
| 12010 | 2P | BG21NUC-HC64 |
| 12011 | 2P | BG21NUC-HC65 |
| 12012 | RBD | BG21NUC-HC66 |
| 12013 | RBD | BG21NUC-HC67 |
| 12014 | RBD | BG21NUC-HC68 |
| 12015 | 2P | BG21NUC-HC69 |
| 12016 | RBD | BG21NUC-HC70 |
| 12017 | RBD | BG21NUC-HC71 |
| 12018 | 2P | BG21NUC-HC72 |
| 12019 | RBD | BG21NUC-HC73 |
| 12020 | 2P | BG21NUC-HC74 |
| 12021 | 2P | BG21NUC-HC75 |
| 12022 | 2P | BG21NUC-HC76 |
| 12023 | 2P | BG21NUC-HC77 |
| 12024 | 2P | BG21NUC-HC78 |
| 12025 | 2P | BG21NUC-HC79 |
| 12026 | 2P | BG21NUC-HC80 |
| 12027 | RBD | BG21NUC-HC81 |
| 12028 | 2P | BG21NUC-HC82 |
| 12029 | 2P | BG21NUC-HC83 |
| 12030 | 2P | BG21NUC-HC84 |
| 12031 | 2P | BG21NUC-HC85 |
| 12032 | 2P | BG21NUC-HC86 |
| 12033 | 2P | BG21NUC-HC87 |
| 12034 | 2P | BG21NUC-HC88 |
| 12035 | RBD | BG21NUC-HC89 |
| 12036 | 2P | BG21NUC-HC90 |
| 12037 | 2P | BG21NUC-HC91 |
| 12038 | 2P | BG21NUC-HC92 |
| 12039 | 2P | BG21NUC-HC93 |
| 12040 | 2P | BG21NUC-HC94 |
| 12041 | RBD | BG21NUC-HC95 |
| 12042 | 2P | BG21NUC-HC96 |
| 12043 | 2P | BG21NUC-HC97 |
| 12044 | 2P | BG21NUC-HC98 |
| 12045 | 2P | BG21NUC-HC99 |
| 12046 | 2P | BG21NUC-HC100 |
| 12047 | 2P | BG21NUC-HC101 |
| 12048 | 2P | BG21NUC-HC102 |
| 12049 | RBD | BG21NUC-HC103 |
| 12050 | RBD | BG21NUC-HC104 |
| 12051 | RBD | BG21NUC-HC105 |
| 12052 | RBD | BG21NUC-HC106 |
| 12053 | 2P | BG21NUC-HC107 |
| 12054 | 2P | BG21NUC-HC108 |
| 12055 | 2P | BG21NUC-HC109 |
| 12056 | 2P | BG21NUC-HC110 |
| 12057 | 2P | BG21NUC-HC111 |
| 12058 | 2P | BG21NUC-HC112 |
| 12059 | 2P | BG21NUC-HC113 |
| 12060 | 2P | BG21NUC-HC114 |
| 12061 | 2P | BG21NUC-HC115 |
| 12062 | 2P | BG21NUC-HC116 |
| 12063 | 2P | BG21NUC-HC117 |
| 12064 | 2P | BG21NUC-HC118 |
| 12065 | 2P | BG21NUC-HC119 |
| 12066 | RBD | BG21NUC-HC120 |
| 12067 | 2P | BG21NUC-HC121 |
| 12068 | 2P | BG21NUC-HC122 |
| 12069 | 2P | BG21NUC-HC123 |
| 12070 | 2P | BG21NUC-HC124 |
| 12071 | RBD | BG21NUC-HC125 |
| 12072 | 2P | BG21NUC-HC126 |
| 12073 | 2P | BG21NUC-HC127 |
| 12074 | 2P | BG21NUC-HC128 |
| 12075 | 2P | BG21NUC-HC129 |
| 12076 | RBD | BG21NUC-HC130 |
| 12077 | RBD | BG21NUC-HC131 |
| 12078 | RBD | BG21NUC-HC132 |
| 12079 | 2P | BG21NUC-HC133 |
| 12080 | 2P | BG21NUC-HC134 |
| 12081 | RBD | BG21NUC-HC135 |
| 12082 | 2P | BG21NUC-HC136 |
| 12083 | 2P | BG21NUC-HC137 |
| 12084 | RBD | BG21NUC-HC138 |
| 12085 | 2P | BG21NUC-HC139 |
| 12086 | 2P | BG21NUC-HC140 |
| 12087 | 2P | BG21NUC-HC141 |
| 12088 | RBD | BG21NUC-HC142 |
| 12089 | 2P | BG21NUC-HC143 |
| 12090 | 2P | BG21NUC-HC144 |
| 12091 | 2P | BG21NUC-HC145 |
| 12092 | 2F | BG21NUC-HC146 |
| 12093 | 2P | BG21NUC-HC147 |
| 12094 | RBD | BG21NUC-HC148 |
| 12095 | RBD | BG21NUC-HC149 |
| 12096 | RBD | BG21NUC-HC150 |
| 12097 | 2P | BG21NUC-HC151 |
| 12098 | 2P | BG21NUC-HC152 |
| 12099 | 2P | BG21NUC-HC153 |
| 12100 | 2P | BG21NUC-HC154 |
| 12101 | 2P | BG21NUC-HC155 |
| 12102 | RBD | BG21NUC-HC156 |
| 12103 | RBD | BG21NUC-HC157 |
| 12104 | RBD | BG21NUC-HC158 |
| 12105 | RBD | BG21NUC-HC159 |
| 12106 | RBD | BG21NUC-HC160 |
| 12107 | 2P | BG21NUC-HC161 |
| 12108 | 2P | BG21NUC-HC162 |
| 12109 | RBD | BG21NUC-HC163 |
| 12110 | 2P | BG21NUC-HC164 |
| 12111 | 2P | BG21NUC-HC165 |
| 12112 | RBD | BG21NUC-HC166 |
| 12113 | 2P | BG21NUC-HC167 |
| 12114 | 2P | BG21NUC-HC168 |
| 12115 | 2P | BG21NUC-HC169 |
| 12116 | 2P | BG21NUC-HC170 |
| 12117 | 2P | BG21NUC-HC171 |
| 12118 | 2P | BG21NUC-HC172 |
| 12119 | 2P | BG21NUC-HC173 |
| 12120 | 2P | BG21NUC-HC174 |
| 12121 | 2P | BG21NUC-HC175 |
| 12122 | 2P | BG21NUC-HC176 |
| 12123 | 2P | BG21NUC-HC177 |
| 12124 | 2P | BG21NUC-HC178 |
| 12125 | 2P | BG21NUC-HC179 |
| 12126 | 2P | BG21NUC-HC180 |
| 12127 | 2P | BG21NUC-HC181 |
| 12128 | 2P | BG21NUC-HC182 |
| 12129 | 2P | BG21NUC-HC183 |
| 12130 | RBD | BG21NUC-HC184 |
| 12131 | 2P | BG21NUC-HC185 |
| 12132 | 2P | BG21NUC-HC186 |
| 12133 | RBD | BG21NUC-HC187 |
| 12134 | RBD | BG21NUC-HC188 |
| 12135 | RBD | BG21NUC-HC189 |
| 12136 | 2P | BG21NUC-HC190 |
| 12137 | 2P | BG21NUC-HC191 |
| 12138 | RBD | BG21NUC-HC192 |
| 12139 | RBD | BG21NUC-HC193 |
| 12140 | 2P | BG21NUC-HC194 |
| 12141 | 2P | BG21NUC-HC195 |
| 12142 | 2P | BG21NUC-HC196 |
| 12143 | 2P | BG21NUC-HC197 |
| 12144 | 2P | BG21NUC-HC198 |
| 12145 | 2P | BG21NUC-HC199 |
| 12146 | RBD | BG21NUC-HC200 |
| 12147 | RBD | BG21NUC-HC201 |
| 12148 | 2P | BG21NUC-HC202 |
| 12149 | RBD | BG21NUC-HC203 |
| 12150 | 2P | BG21NUC-HC204 |
| 12151 | 2P | BG21NUC-HC205 |
| 12152 | 2P | BG21NUC-HC206 |
| 12153 | 2P | BG21NUC-HC207 |
| 12154 | 2P | BG21NUC-HC208 |
| 12155 | 2P | BG21NUC-HC209 |
| 12156 | 2P | BG21NUC-HC210 |
| 12157 | RBD | BG21NUC-HC211 |
| 12158 | 2P | BG21NUC-HC212 |
| 12159 | 2P | BG21NUC-HC213 |
| 12160 | 2P | BG21NUC-HC214 |
| 12161 | 2P | BG21NUC-HC215 |
| 12162 | 2P | BG21NUC-HC216 |
| 12163 | 2P | BG21NUC-HC217 |
| 12164 | 2P | BG21NUC-HC218 |
| 12165 | RBD | BG21NUC-HC219 |
| 12166 | 2P | BG21NUC-HC220 |
| 12167 | RBD | BG21NUC-HC221 |
| 12168 | 2P | BG21NUC-HC222 |
| 12169 | RBD | BG21NUC-HC223 |
| 12170 | 2P | BG21NUC-HC224 |
| 12171 | 2P | BG21NUC-HC225 |
| 12172 | 2P | BG21NUC-HC226 |
| 12173 | 2P | BG21NUC-HC227 |
| 12174 | 2P | BG21NUC-HC228 |
| 12175 | 2P | BG21NUC-HC229 |
| 12176 | 2P | BG21NUC-HC230 |
| 12177 | 2P | BG21NUC-HC231 |
| 12178 | 2P | BG21NUC-HC232 |
| 12179 | 2P | BG21NUC-HC233 |
| 12180 | 2P | BG21NUC-HC234 |
| 12181 | 2P | BG21NUC-HC235 |
| 12182 | 2P | BG21NUC-HC236 |
| 12183 | 2P | BG21NUC-HC237 |
| 12184 | 2P | BG21NUC-HC238 |
| 12185 | 2P | BG21NUC-HC239 |
| 12186 | RBD | BG21NUC-HC240 |
| 12187 | 2P | BG21NUC-HC241 |
| 12188 | 2P | BG21NUC-HC242 |
| 12189 | RBD | BG21NUC-HC243 |
| 12190 | RBD | BG21NUC-HC244 |
| 12191 | 2P | BG21NUC-HC245 |
| 12192 | RBD | BG21NUC-HC246 |
| 12193 | RBD | BG21NUC-HC247 |
| 12194 | RBD | BG21NUC-HC248 |
| 12195 | 2P | BG21NUC-HC249 |
| 12196 | 2P | BG21NUC-HC250 |
| 12197 | RBD | BG21NUC-HC251 |
| 12198 | 2P | BG21NUC-HC252 |
| 12199 | 2P | BG21NUC-HC253 |
| 12200 | 2P | BG21NUC-HC254 |
| 12201 | 2P | BG21NUC-HC255 |
| 12202 | 2P | BG21NUC-HC256 |
| 12203 | RBD | BG21NUC-HC257 |
| 12204 | 2P | BG21NUC-HC258 |
| 12205 | 2P | BG21NUC-HC259 |
| 12206 | 2P | BG21NUC-HC260 |
| 12207 | 2P | BG21NUC-HC261 |
| 12208 | 2P | BG21NUC-HC262 |
| 12209 | RBD | BG21NUC-HC263 |
| 12210 | 2P | BG21NUC-HC264 |
| 12211 | 2P | BG21NUC-HC265 |
| 12212 | RBD | BG21NUC-HC266 |
| 12213 | 2P | BG21NUC-HC267 |
| 12214 | 2P | BG21NUC-HC268 |
| 12215 | 2P | BG21NUC-HC269 |
| 12216 | 2P | BG21NUC-HC270 |
| 12217 | 2P | BG21NUC-HC271 |
| 12218 | 2P | BG21NUC-HC272 |
| 12219 | 2P | BG21NUC-HC273 |
| 12220 | 2P | BG21NUC-HC274 |
| 12221 | 2P | BG21NUC-HC275 |
| 12222 | 2P | BG21NUC-HC276 |
| 12223 | 2P | BG21NUC-HC277 |
| 12224 | 2P | BG21NUC-HC278 |
| 12225 | 2P | BG21NUC-HC279 |
| 12226 | RBD | BG21NUC-HC280 |
| 12227 | 2P | BG21NUC-HC281 |
| 12228 | RBD | BG21NUC-HC282 |
| 12229 | 2P | BG21NUC-HC283 |
| 12230 | 2P | BG21NUC-HC284 |
| 12231 | RBD | BG21NUC-HC285 |
| 12232 | 2P | BG21NUC-HC286 |
| 12233 | RBD | BG21NUC-HC287 |
| 12234 | RBD | BG21NUC-HC288 |
| 12235 | 2P | BG21NUC-HC289 |
| 12236 | 2P | BG21NUC-HC290 |
| 12237 | 2P | BG21NUC-HC291 |
| 12238 | RBD | BG21NUC-HC292 |
| 12239 | 2P | BG21NUC-HC293 |
| 12240 | 2P | BG21NUC-HC294 |
| 12241 | RBD | BG21NUC-HC295 |
| 12242 | 2P | BG21NUC-HC296 |
| 12243 | 2P | BG21NUC-HC297 |
| 12244 | 2P | BG21NUC-HC298 |
| 12245 | 2P | BG21NUC-HC299 |
| 12246 | 2P | BG21NUC-HC300 |
| 12247 | 2P | BG21NUC-HC301 |
| 12248 | 2P | BG21NUC-HC302 |
| 12249 | 2P | BG21NUC-HC303 |
| 12250 | 2P | BG21NUC-HC304 |
| 12251 | RBD | BG21NUC-HC305 |
| 12252 | 2P | BG21NUC-HC306 |
| 12253 | 2P | BG21NUC-HC307 |
| 12254 | RBD | BG21NUC-HC308 |
| 12255 | RBD | BG21NUC-HC309 |
| 12256 | RBD | BG21NUC-HC310 |
| 12257 | 2P | BG21NUC-HC311 |
| 12258 | 2P | BG21NUC-HC312 |
| 12259 | 2P | BG21NUC-HC313 |
| 12260 | 2P | BG21NUC-HC314 |
| 12261 | RBD | BG21NUC-HC315 |
| 12262 | 2P | BG21NUC-HC316 |
| 12263 | RBD | BG21NUC-HC317 |
| 12264 | 2P | BG21NUC-HC318 |
| 12265 | 2P | BG21NUC-HC319 |
| 12266 | 2P | BG21NUC-HC320 |
| 12267 | RBD | BG21NUC-HC321 |
| 12268 | 2F | BG21NUC-HC322 |
| 12269 | 2P | BG21NUC-HC323 |
| 12270 | 2P | BG21NUC-HC324 |
| 12271 | 2P | BG21NUC-HC325 |
| 12272 | RBD | BG21NUC-HC326 |
| 12273 | 2P | BG21NUC-HC327 |
| 12274 | RBD | BG21NUC-HC328 |
| 12275 | 2P | BG21NUC-HC329 |
| 12276 | 2P | BG21NUC-HC330 |
| 12277 | 2P | BG21NUC-HC331 |
| 12278 | 2P | BG21NUC-HC332 |
| 12279 | 2P | BG21NUC-HC333 |
| 12280 | 2P | BG21NUC-HC334 |
| 12281 | RBD | BG21NUC-HC335 |
| 12282 | 2P | BG21NUC-HC336 |
| 12283 | RBD | BG21NUC-HC337 |
| 12284 | RBD | BG21NUC-HC338 |
| 12285 | RBD | BG21NUC-HC339 |
| 12286 | 2P | BG21NUC-HC340 |
| 12287 | 2P | BG21NUC-HC341 |
| 12288 | 2P | BG21NUC-HC342 |
| 12289 | 2P | BG21NUC-HC343 |
| 12290 | 2P | BG21NUC-HC344 |
| 12291 | RBD | BG21NUC-HC345 |
| 12292 | 2P | BG21NUC-HC346 |
| 12293 | 2P | BG21NUC-HC347 |
| 12294 | 2P | BG21NUC-HC348 |
| 12295 | 2P | BG21NUC-HC349 |
| 12296 | 2P | BG21NUC-HC350 |
| 12297 | 2P | BG21NUC-HC351 |
| 12298 | 2P | BG21NUC-HC352 |
| 12299 | RBD | BG21NUC-HC353 |
| 12300 | 2P | BG21NUC-HC354 |
| 12301 | 2P | BG21NUC-HC355 |
| 12302 | 2P | BG21NUC-HC356 |
| 12303 | 2P | BG21NUC-HC357 |
| 12304 | 2P | BG21NUC-HC358 |
| 12305 | 2P | BG21NUC-HC359 |
| 12306 | 2P | BG21NUC-HC360 |
| 12307 | RBD | BG21NUC-HC361 |
| 12308 | 2F | BG21NUC-HC362 |
| 12309 | 2P | BG21NUC-HC363 |
| 12310 | 2P | BG21NUC-LC1 |
| 12311 | 2P | BG21NUC-LC2 |
| 12312 | RBD | BG21NUC-LC3 |
| 12313 | 2P | BG21NUC-LC4 |
| 12314 | 2P | BG21NUC-LC5 |
| 12315 | 2P | BG21NUC-LC6 |
| 12316 | 2P | BG21NUC-LC7 |
| 12317 | 2P | BG21NUC-LC8 |
| 12318 | 2P | BG21NUC-LC9 |
| 12319 | 2P | BG21NUC-LC10 |
| 12320 | 2F | BG21NUC-LC11 |
| 12321 | RBD | BG21NUC-LC12 |
| 12322 | 2P | BG21NUC-LC13 |
| 12323 | 2P | BG21NUC-LC14 |
| 12324 | 2P | BG21NUC-LC15 |
| 12325 | 2P | BG21NUC-LC16 |
| 12326 | 2P | BG21NUC-LC17 |
| 12327 | 2P | BG21NUC-LC18 |
| 12328 | 2P | BG21NUC-LC19 |
| 12329 | 2P | BG21NUC-LC20 |
| 12330 | 2P | BG21NUC-LC21 |
| 12331 | RBD | BG21NUC-LC22 |
| 12332 | 2P | BG21NUC-LC23 |
| 12333 | 2F | BG21NUC-LC24 |
| 12334 | 2P | BG21NUC-LC25 |
| 12335 | 2P | BG21NUC-LC26 |
| 12336 | RBD | BG21NUC-LC27 |
| 12337 | 2P | BG21NUC-LC28 |
| 12338 | 2P | BG21NUC-LC29 |
| 12339 | 2P | BG21NUC-LC30 |
| 12340 | 2P | BG21NUC-LC31 |
| 12341 | 2P | BG21NUC-LC32 |
| 12342 | RBD | BG21NUC-LC33 |
| 12343 | 2P | BG21NUC-LC34 |
| 12344 | 2P | BG21NUC-LC35 |
| 12345 | RBD | BG21NUC-LC36 |
| 12346 | 2P | BG21NUC-LC37 |
| 12347 | 2P | BG21NUC-LC38 |
| 12348 | 2P | BG21NUC-LC39 |
| 12349 | 2P | BG21NUC-LC40 |
| 12350 | 2P | BG21NUC-LC41 |
| 12351 | RBD | BG21NUC-LC42 |
| 12352 | 2P | BG21NUC-LC43 |
| 12353 | 2P | BG21NUC-LC44 |
| 12354 | RBD | BG21NUC-LC45 |
| 12355 | 2P | BG21NUC-LC46 |
| 12356 | 2P | BG21NUC-LC47 |
| 12357 | 2P | BG21NUC-LC48 |
| 12358 | 2P | BG21NUC-LC49 |
| 12359 | 2P | BG21NUC-LC50 |
| 12360 | RBD | BG21NUC-LC51 |
| 12361 | RBD | BG21NUC-LC52 |
| 12362 | 2P | BG21NUC-LC53 |
| 12363 | RBD | BG21NUC-LC54 |
| 12364 | 2P | BG21NUC-LC55 |
| 12365 | RBD | BG21NUC-LC56 |
| 12366 | 2P | BG21NUC-LC57 |
| 12367 | RBD | BG21NUC-LC58 |
| 12368 | RBD | BG21NUC-LC59 |
| 12369 | RBD | BG21NUC-LC60 |
| 12370 | RBD | BG21NUC-LC61 |
| 12371 | RBD | BG21NUC-LC62 |
| 12372 | RBD | BG21NUC-LC63 |
| 12373 | 2P | BG21NUC-LC64 |
| 12374 | 2P | BG21NUC-LC65 |
| 12375 | RBD | BG21NUC-LC66 |
| 12376 | RBD | BG21NUC-LC67 |
| 12377 | RBD | BG21NUC-LC68 |
| 12378 | 2P | BG21NUC-LC69 |
| 12379 | RBD | BG21NUC-LC70 |
| 12380 | RBD | BG21NUC-LC71 |
| 12381 | 2P | BG21NUC-LC72 |
| 12382 | RBD | BG21NUC-LC73 |
| 12383 | 2P | BG21NUC-LC74 |
| 12384 | 2P | BG21NUC-LC75 |
| 12385 | 2P | BG21NUC-LC76 |
| 12386 | 2P | BG21NUC-LC77 |
| 12387 | 2P | BG21NUC-LC78 |
| 12388 | 2P | BG21NUC-LC79 |
| 12389 | 2P | BG21NUC-LC80 |
| 12390 | RBD | BG21NUC-LC81 |
| 12391 | 2P | BG21NUC-LC82 |
| 12392 | 2P | BG21NUC-LC83 |
| 12393 | 2P | BG21NUC-LC84 |
| 12394 | 2P | BG21NUC-LC85 |
| 12395 | 2P | BG21NUC-LC86 |
| 12396 | 2P | BG21NUC-LC87 |
| 12397 | 2P | BG21NUC-LC88 |
| 12398 | RBD | BG21NUC-LC89 |
| 12399 | 2P | BG21NUC-LC90 |
| 12400 | 2P | BG21NUC-LC91 |
| 12401 | 2P | BG21NUC-LC92 |
| 12402 | 2P | BG21NUC-LC93 |
| 12403 | 2P | BG21NUC-LC94 |
| 12404 | RBD | BG21NUC-LC95 |
| 12405 | 2P | BG21NUC-LC96 |
| 12406 | 2P | BG21NUC-LC97 |
| 12407 | 2P | BG21NUC-LC98 |
| 12408 | 2P | BG21NUC-LC99 |
| 12409 | 2P | BG21NUC-LC100 |
| 12410 | 2P | BG21NUC-LC101 |
| 12411 | 2P | BG21NUC-LC102 |
| 12412 | RBD | BG21NUC-LC103 |
| 12413 | RBD | BG21NUC-LC104 |
| 12414 | RBD | BG21NUC-LC105 |
| 12415 | RBD | BG21NUC-LC106 |
| 12416 | 2P | BG21NUC-LC107 |
| 12417 | 2P | BG21NUC-LC108 |
| 12418 | 2P | BG21NUC-LC109 |
| 12419 | 2P | BG21NUC-LC110 |
| 12420 | 2P | BG21NUC-LC111 |
| 12421 | 2P | BG21NUC-LC112 |
| 12422 | 2P | BG21NUC-LC113 |
| 12423 | 2P | BG21NUC-LC114 |
| 12424 | 2P | BG21NUC-LC115 |
| 12425 | 2P | BG21NUC-LC116 |
| 12426 | 2P | BG21NUC-LC117 |
| 12427 | 2P | BG21NUC-LC118 |
| 12428 | 2P | BG21NUC-LC119 |
| 12429 | RBD | BG21NUC-LC120 |
| 12430 | 2P | BG21NUC-LC121 |
| 12431 | 2P | BG21NUC-LC122 |
| 12432 | 2P | BG21NUC-LC123 |
| 12433 | 2P | BG21NUC-LC124 |
| 12434 | RBD | BG21NUC-LC125 |
| 12435 | 2P | BG21NUC-LC126 |
| 12436 | 2P | BG21NUC-LC127 |
| 12437 | 2P | BG21NUC-LC128 |
| 12438 | 2P | BG21NUC-LC129 |
| 12439 | RBD | BG21NUC-LC130 |
| 12440 | RBD | BG21NUC-LC131 |
| 12441 | RBD | BG21NUC-LC132 |
| 12442 | 2P | BG21NUC-LC133 |
| 12443 | 2P | BG21NUC-LC134 |
| 12444 | RBD | BG21NUC-LC135 |
| 12445 | 2P | BG21NUC-LC136 |
| 12446 | 2P | BG21NUC-LC137 |
| 12447 | RBD | BG21NUC-LC138 |
| 12448 | 2P | BG21NUC-LC139 |
| 12449 | 2P | BG21NUC-LC140 |
| 12450 | 2P | BG21NUC-LC141 |
| 12451 | RBD | BG21NUC-LC142 |
| 12452 | 2P | BG21NUC-LC143 |
| 12453 | 2P | BG21NUC-LC144 |
| 12454 | 2P | BG21NUC-LC145 |
| 12455 | 2P | BG21NUC-LC146 |
| 12456 | 2F | BG21NUC-LC147 |
| 12457 | RBD | BG21NUC-LC148 |
| 12458 | RBD | BG21NUC-LC149 |
| 12459 | RBD | BG21NUC-LC150 |
| 12460 | 2P | BG21NUC-LC151 |
| 12461 | 2P | BG21NUC-LC152 |
| 12462 | 2P | BG21NUC-LC153 |
| 12463 | 2P | BG21NUC-LC154 |
| 12464 | 2P | BG21NUC-LC155 |
| 12465 | RBD | BG21NUC-LC156 |
| 12466 | RBD | BG21NUC-LC157 |
| 12467 | RBD | BG21NUC-LC158 |
| 12468 | RBD | BG21NUC-LC159 |
| 12469 | RBD | BG21NUC-LC160 |
| 12470 | 2P | BG21NUC-LC161 |
| 12471 | 2P | BG21NUC-LC162 |
| 12472 | RBD | BG21NUC-LC163 |
| 12473 | 2P | BG21NUC-LC164 |
| 12474 | 2P | BG21NUC-LC165 |
| 12475 | RBD | BG21NUC-LC166 |
| 12476 | 2P | BG21NUC-LC167 |
| 12477 | 2P | BG21NUC-LC168 |
| 12478 | 2P | BG21NUC-LC169 |
| 12479 | 2P | BG21NUC-LC170 |
| 12480 | 2P | BG21NUC-LC171 |
| 12481 | 2P | BG21NUC-LC172 |
| 12482 | 2P | BG21NUC-LC173 |
| 12483 | 2P | BG21NUC-LC174 |
| 12484 | 2F | BG21NUC-LC175 |
| 12485 | 2F | BG21NUC-LC176 |
| 12486 | 2P | BG21NUC-LC177 |
| 12487 | 2P | BG21NUC-LC178 |
| 12488 | 2F | BG21NUC-LC179 |
| 12489 | 2F | BG21NUC-LC180 |
| 12490 | 2F | BG21NUC-LC181 |
| 12491 | 2P | BG21NUC-LC182 |
| 12492 | 2P | BG21NUC-LC183 |
| 12493 | RBD | BG21NUC-LC184 |
| 12494 | 2P | BG21NUC-LC185 |
| 12495 | 2P | BG21NUC-LC186 |
| 12496 | RBD | BG21NUC-LC187 |
| 12497 | RBD | BG21NUC-LC188 |
| 12498 | RBD | BG21NUC-LC189 |
| 12499 | 2P | BG21NUC-LC190 |
| 12500 | 2P | BG21NUC-LC191 |
| 12501 | RBD | BG21NUC-LC192 |
| 12502 | RBD | BG21NUC-LC193 |
| 12503 | 2P | BG21NUC-LC194 |
| 12504 | 2P | BG21NUC-LC195 |
| 12505 | 2P | BG21NUC-LC196 |
| 12506 | 2F | BG21NUC-LC197 |
| 12507 | 2P | BG21NUC-LC198 |
| 12508 | 2P | BG21NUC-LC199 |
| 12509 | RBD | BG21NUC-LC200 |
| 12510 | RBD | BG21NUC-LC201 |
| 12511 | 2P | BG21NUC-LC202 |
| 12512 | RBD | BG21NUC-LC203 |
| 12513 | 2P | BG21NUC-LC204 |
| 12514 | 2F | BG21NUC-LC205 |
| 12515 | 2P | BG21NUC-LC206 |
| 12516 | 2P | BG21NUC-LC207 |
| 12517 | 2P | BG21NUC-LC208 |
| 12518 | 2F | BG21NUC-LC209 |
| 12519 | 2P | BG21NUC-LC210 |
| 12520 | RBD | BG21NUC-LC211 |
| 12521 | 2P | BG21NUC-LC212 |
| 12522 | 2F | BG21NUC-LC213 |
| 12523 | 2P | BG21NUC-LC214 |
| 12524 | 2P | BG21NUC-LC215 |
| 12525 | 2P | BG21NUC-LC216 |
| 12526 | 2P | BG21NUC-LC217 |
| 12527 | 2P | BG21NUC-LC218 |
| 12528 | RBD | BG21NUC-LC219 |
| 12529 | 2P | BG21NUC-LC220 |
| 12530 | RBD | BG21NUC-LC221 |
| 12531 | 2P | BG21NUC-LC222 |
| 12532 | RBD | BG21NUC-LC223 |
| 12533 | 2P | BG21NUC-LC224 |
| 12534 | 2F | BG21NUC-LC225 |
| 12535 | 2P | BG21NUC-LC226 |
| 12536 | 2F | BG21NUC-LC227 |
| 12537 | 2F | BG21NUC-LC228 |
| 12538 | 2F | BG21NUC-LC229 |
| 12539 | 2F | BG21NUC-LC230 |
| 12540 | 2P | BG21NUC-LC231 |
| 12541 | 2P | BG21NUC-LC232 |
| 12542 | 2P | BG21NUC-LC233 |
| 12543 | 2P | BG21NUC-LC234 |
| 12544 | 2P | BG21NUC-LC235 |
| 12545 | 2P | BG21NUC-LC236 |
| 12546 | 2P | BG21NUC-LC237 |
| 12547 | 2P | BG21NUC-LC238 |
| 12548 | 2P | BG21NUC-LC239 |
| 12549 | RBD | BG21NUC-LC240 |
| 12550 | 2P | BG21NUC-LC241 |
| 12551 | 2P | BG21NUC-LC242 |
| 12552 | RBD | BG21NUC-LC243 |
| 12553 | RBD | BG21NUC-LC244 |
| 12554 | 2P | BG21NUC-LC245 |
| 12555 | RBD | BG21NUC-LC246 |
| 12556 | RBD | BG21NUC-LC247 |
| 12557 | RBD | BG21NUC-LC248 |
| 12558 | 2P | BG21NUC-LC249 |
| 12559 | 2P | BG21NUC-LC250 |
| 12560 | RBD | BG21NUC-LC251 |
| 12561 | 2P | BG21NUC-LC252 |
| 12562 | 2P | BG21NUC-LC253 |
| 12563 | 2P | BG21NUC-LC254 |
| 12564 | 2P | BG21NUC-LC255 |
| 12565 | 2P | BG21NUC-LC256 |
| 12566 | RBD | BG21NUC-LC257 |
| 12567 | 2P | BG21NUC-LC258 |
| 12568 | 2P | BG21NUC-LC259 |
| 12569 | 2P | BG21NUC-LC260 |
| 12570 | 2P | BG21NUC-LC261 |
| 12571 | 2P | BG21NUC-LC262 |
| 12572 | RBD | BG21NUC-LC263 |
| 12573 | 2P | BG21NUC-LC264 |
| 12574 | 2P | BG21NUC-LC265 |
| 12575 | RBD | BG21NUC-LC266 |
| 12576 | 2P | BG21NUC-LC267 |
| 12577 | 2P | BG21NUC-LC268 |
| 12578 | 2P | BG21NUC-LC269 |
| 12579 | 2P | BG21NUC-LC270 |
| 12580 | 2P | BG21NUC-LC271 |
| 12581 | 2P | BG21NUC-LC272 |
| 12582 | 2P | BG21NUC-LC273 |
| 12583 | 2P | BG21NUC-LC274 |
| 12584 | 2P | BG21NUC-LC275 |
| 12585 | 2P | BG21NUC-LC276 |
| 12586 | 2P | BG21NUC-LC277 |
| 12587 | 2P | BG21NUC-LC278 |
| 12588 | 2P | BG21NUC-LC279 |
| 12589 | RBD | BG21NUC-LC280 |
| 12590 | 2P | BG21NUC-LC281 |
| 12591 | RBD | BG21NUC-LC282 |
| 12592 | 2P | BG21NUC-LC283 |
| 12593 | 2P | BG21NUC-LC284 |
| 12594 | RBD | BG21NUC-LC285 |
| 12595 | 2F | BG21NUC-LC286 |
| 12596 | RBD | BG21NUC-LC287 |
| 12597 | RBD | BG21NUC-LC288 |
| 12598 | 2P | BG21NUC-LC289 |
| 12599 | 2P | BG21NUC-LC290 |
| 12600 | 2P | BG21NUC-LC291 |
| 12601 | RBD | BG21NUC-LC292 |
| 12602 | 2P | BG21NUC-LC293 |
| 12603 | 2P | BG21NUC-LC294 |
| 12604 | RBD | BG21NUC-LC295 |
| 12605 | 2P | BG21NUC-LC296 |
| 12606 | 2P | BG21NUC-LC297 |
| 12607 | 2P | BG21NUC-LC298 |
| 12608 | 2P | BG21NUC-LC299 |
| 12609 | 2P | BG21NUC-LC300 |
| 12610 | 2P | BG21NUC-LC301 |
| 12611 | 2P | BG21NUC-LC302 |
| 12612 | 2P | BG21NUC-LC303 |
| 12613 | 2P | BG21NUC-LC304 |
| 12614 | RBD | BG21NUC-LC305 |
| 12615 | 2P | BG21NUC-LC306 |
| 12616 | 2P | BG21NUC-LC307 |
| 12617 | RBD | BG21NUC-LC308 |
| 12618 | RBD | BG21NUC-LC309 |
| 12619 | RBD | BG21NUC-LC310 |
| 12620 | 2P | BG21NUC-LC311 |
| 12621 | 2P | BG21NUC-LC312 |
| 12622 | 2P | BG21NUC-LC313 |
| 12623 | 2P | BG21NUC-LC314 |
| 12624 | RBD | BG21NUC-LC315 |
| 12625 | 2P | BG21NUC-LC316 |
| 12626 | RBD | BG21NUC-LC317 |
| 12627 | 2P | BG21NUC-LC318 |
| 12628 | 2P | BG21NUC-LC319 |
| 12629 | 2P | BG21NUC-LC320 |
| 12630 | RBD | BG21NUC-LC321 |
| 12631 | 2P | BG21NUC-LC322 |
| 12632 | 2F | BG21NUC-LC323 |
| 12633 | 2P | BG21NUC-LC324 |
| 12634 | 2F | BG21NUC-LC325 |
| 12635 | RBD | BG21NUC-LC326 |
| 12636 | 2F | BG21NUC-LC327 |
| 12637 | RBD | BG21NUC-LC328 |
| 12638 | 2F | BG21NUC-LC329 |
| 12639 | 2P | BG21NUC-LC330 |
| 12640 | 2F | BG21NUC-LC331 |
| 12641 | 2P | BG21NUC-LC332 |
| 12642 | 2F | BG21NUC-LC333 |
| 12643 | 2F | BG21NUC-LC334 |
| 12644 | RBD | BG21NUC-LC335 |
| 12645 | 2P | BG21NUC-LC336 |
| 12646 | RBD | BG21NUC-LC337 |
| 12647 | RBD | BG21NUC-LC338 |
| 12648 | RBD | BG21NUC-LC339 |
| 12649 | 2F | BG21NUC-LC340 |
| 12650 | 2P | BG21NUC-LC341 |
| 12651 | 2F | BG21NUC-LC342 |
| 12652 | 2P | BG21NUC-LC343 |
| 12653 | 2F | BG21NUC-LC344 |
| 12654 | RBD | BG21NUC-LC345 |
| 12655 | 2P | BG21NUC-LC346 |
| 12656 | 2P | BG21NUC-LC347 |
| 12657 | 2F | BG21NUC-LC348 |
| 12658 | 2P | BG21NUC-LC349 |
| 12659 | 2F | BG21NUC-LC350 |
| 12660 | 2P | BG21NUC-LC351 |
| 12661 | 2F | BG21NUC-LC352 |
| 12662 | RBD | BG21NUC-LC353 |
| 12663 | 2F | BG21NUC-LC354 |
| 12664 | 2F | BG21NUC-LC355 |
| 12665 | 2F | BG21NUC-LC356 |
| 12666 | 2P | BG21NUC-LC357 |
| 12667 | 2P | BG21NUC-LC358 |
| 12668 | 2P | BG21NUC-LC359 |
| 12669 | 2P | BG21NUC-LC360 |
| 12670 | RBD | BG21NUC-LC361 |
| 12671 | 2F | BG21NUC-LC362 |
| 12672 | 2P | BG21NUC-LC363 |

TABLE 8B

| SEQ | BAIT | | Produced | SEQ | BAIT | | Produced |
|---|---|---|---|---|---|---|---|
| ID NO | USED | SEQUENCE NAME | Antibody Name | ID NO | USED | SEQUENCE NAME | Antibody Name |
| 447 | RBD | BG1NUC-HC1 | BG1-1 | 2271 | 2P | BG4NUC-LC40 | BG4-12 |
| 456 | RBD | BG1NUC-HC10 | BG1-3 | 2273 | RBD | BG4NUC-LC42 | BG4-13 |
| 459 | RBD | BG1NUC-HC13 | BG1-4 | 2275 | 2P | BG4NUC-LC44 | BG4-14 |
| 462 | RBD | BG1NUC-HC16 | BG1-5 | 2277 | 2P | BG4NUC-LC46 | BG4-15 |
| 466 | RBD | BG1NUC-HC20 | BG1-6 | 2279 | RBD | BG4NUC-LC48 | BG4-16 |
| 467 | 2P | BG1NUC-HC21 | BG1-7 | 2281 | 2P | BG4NUC-LC50 | BG4-17 |
| 469 | 2P | BG1NUC-HC23 | BG1-8 | 2283 | 2P | BG4NUC-LC52 | BG4-18 |
| 471 | RBD | BG1NUC-HC25 | BG1-9 | 2285 | RBD | BG4NUC-LC54 | BG4-19 |
| 473 | RBD | BG1NUC-HC27 | BG1-10 | 2288 | 2P | BG4NUC-LC57 | BG4-20 |
| 475 | RBD | BG1NUC-HC29 | BG1-11 | 2289 | RBD | BG4NUC-LC58 | BG4-21 |
| 477 | RBD | BG1NUC-HC31 | BG1-12 | 2364 | 2P | BG4NUC-LC133 | BG4-22 |
| 479 | 2P | BG1NUC-HC33 | BG1-13 | 2389 | RBD | BG4NUC-LC158 | BG4-23 |
| 481 | RBD | BG1NUC-HC35 | BG1-14 | 2448 | RBD | BG4NUC-LC217 | BG4-24 |
| 483 | RBD | BG1NUC-HC37 | BG1-15 | 2460 | 2P | BG4NUC-LC229 | BG4-25 |
| 485 | 2P | BG1NUC-HC39 | BG1-16 | 2537 | RBD | BG4NUC-LC306 | BG4-26 |
| 487 | RBD | BG1NUC-HC41 | BG1-17 | 2614 | 2P | BG4NUC-LC383 | BG4-27 |
| 489 | 2F | BG1NUC-HC43 | BG1-18 | 4085 | 2P | BG7NUC-HC1 | BG7-1 |
| 514 | RBD | BG1NUC-HC68 | BG1-19 | 4097 | 2P | BG7NUC-HC13 | BG7-2 |
| 528 | 2P | BG1NUC-HC82 | BG1-20 | 4106 | 2P | BG7NUC-HC22 | BG7-3 |
| 536 | RBD | BG1NUC-HC90 | BG1-21 | 4115 | 2P | BG7NUC-HC31 | BG7-4 |
| 542 | RBD | BG1NUC-HC96 | BG1-22 | 4121 | 2P | BG7NUC-HC37 | BG7-5 |
| 548 | RBD | BG1NUC-HC102 | BG1-23 | 4126 | 2P | BG7NUC-HC42 | BG7-6 |
| 571 | RBD | BG1NUC-HC125 | BG1-24 | 4129 | 2P | BG7NUC-HC45 | BG7-8 |
| 602 | RBD | BG1NUC-HC156 | BG1-25 | 4132 | 2P | BG7NUC-HC48 | BG7-9 |
| 634 | RBD | BG1NUC-HC188 | BG1-26 | 4134 | 2P | BG7NUC-HC50 | BG7-10 |
| 690 | RBD | BG1NUC-HC244 | BG1-27 | 4136 | 2P | BG7NUC-HC52 | BG7-11 |
| 701 | RBD | BG1NUC-HC255 | BG1-28 | 4138 | 2P | BG7NUC-HC54 | BG7-12 |
| 755 | RBD | BG1NUC-LC1 | BG1-1 | 4140 | 2P | BG7NUC-HC56 | BG7-13 |
| 764 | RBD | BG1NUC-LC10 | BG1-3 | 4346 | RBD | BG7NUC-HC262 | BG7-14 |
| 767 | RBD | BG1NUC-LC13 | BG1-4 | 4369 | RBD | BG7NUC-HC285 | BG7-15 |
| 770 | RBD | BG1NUC-LC16 | BG1-5 | 4372 | RBD | BG7NUC-HC288 | BG7-16 |
| 774 | RBD | BG1NUC-LC20 | BG1-6 | 4386 | RBD | BG7NUC-HC302 | BG7-17 |
| 775 | 2P | BG1NUC-LC21 | BG1-7 | 4393 | RBD | BG7NUC-HC309 | BG7-18 |
| 777 | 2P | BG1NUC-LC23 | BG1-8 | 4402 | RBD | BG7NUC-HC318 | BG7-19 |
| 779 | RBD | BG1NUC-LC25 | BG1-9 | 4414 | RBD | BG7NUC-HC330 | BG7-20 |
| 781 | RBD | BG1NUC-LC27 | BG1-10 | 4482 | 2P | BG7NUC-LC1 | BG7-1 |
| 783 | RBD | BG1NUC-LC29 | BG1-11 | 4494 | 2P | BG7NUC-LC13 | BG7-2 |
| 785 | RBD | BG1NUC-LC31 | BG1-12 | 4503 | 2P | BG7NUC-LC22 | BG7-3 |
| 787 | 2P | BG1NUC-LC33 | BG1-13 | 4512 | 2P | BG7NUC-LC31 | BG7-4 |
| 789 | RBD | BG1NUC-LC35 | BG1-14 | 4518 | 2P | BG7NUC-LC37 | BG7-5 |
| 791 | RBD | BG1NUC-LC37 | BG1-15 | 4523 | 2P | BG7NUC-LC42 | BG7-6 |
| 793 | 2P | BG1NUC-LC39 | BG1-16 | 4526 | 2P | BG7NUC-LC45 | BG7-8 |
| 795 | RBD | BG1NUC-LC41 | BG1-17 | 4529 | 2P | BG7NUC-LC48 | BG7-9 |
| 797 | 2P | BG1NUC-LC43 | BG1-18 | 4531 | 2P | BG7NUC-LC50 | BG7-10 |
| 822 | RBD | BG1NUC-LC68 | BG1-19 | 4533 | 2P | BG7NUC-LC52 | BG7-11 |
| 836 | 2P | BG1NUC-LC82 | BG1-20 | 4535 | 2P | BG7NUC-LC54 | BG7-12 |
| 844 | RBD | BG1NUC-LC90 | BG1-21 | 4537 | 2P | BG7NUC-LC56 | BG7-13 |
| 850 | RBD | BG1NUC-LC96 | BG1-22 | 4743 | RBD | BG7NUC-LC262 | BG7-14 |
| 856 | RBD | BG1NUC-LC102 | BG1-23 | 4766 | RBD | BG7NUC-LC285 | BG7-15 |
| 879 | RBD | BG1NUC-LC125 | BG1-24 | 4769 | RBD | BG7NUC-LC288 | BG7-16 |
| 910 | RBD | BG1NUC-LC156 | BG1-25 | 4783 | RBD | BG7NUC-LC302 | BG7-17 |
| 942 | RBD | BG1NUC-LC188 | BG1-26 | 4790 | RBD | BG7NUC-LC309 | BG7-18 |
| 998 | RBD | BG1NUC-LC244 | BG1-27 | 4799 | RBD | BG7NUC-LC318 | BG7-19 |
| 1009 | RBD | BG1NUC-LC255 | BG1-28 | 4811 | RBD | BG7NUC-LC330 | BG7-20 |
| 1823 | 2F | BG4NUC-HC1 | BG4-1 | 5347 | RBD | BG10NUC-HC1 | BG10-1 |
| 1834 | 2P | BG4NUC-HC12 | BG4-2 | 5383 | RBD | BG10NUC-HC37 | BG10-2 |
| 1838 | 2P | BG4NUC-HC16 | BG4-3 | 5398 | RBD | BG10NUC-HC52 | BG10-3 |
| 1843 | 2F | BG4NUC-HC21 | BG4-4 | 5407 | RBD | BG10NUC-HC61 | BG10-4 |
| 1845 | RBD | BG4NUC-HC23 | BG4-5 | 5416 | RBD | BG10NUC-HC70 | BG10-5 |
| 1847 | RBD | BG4NUC-HC25 | BG4-6 | 5424 | RBD | BG10NUC-HC78 | BG10-6 |
| 1850 | RBD | BG4NUC-HC28 | BG4-7 | 5431 | RBD | BG10NUC-HC85 | BG10-7 |
| 1853 | RBD | BG4NUC-HC31 | BG4-8 | 5436 | RBD | BG10NUC-HC90 | BG10-8 |
| 1856 | RBD | BG4NUC-HC34 | BG4-9 | 5441 | 2P | BG10NUC-HC95 | BG10-9 |
| 1859 | RBD | BG4NUC-HC37 | BG4-10 | 5446 | RBD | BG10NUC-HC100 | BG10-10 |
| 1860 | RBD | BG4NUC-HC38 | BG4-11 | 5450 | RBD | BG10NUC-HC104 | BG10-11 |
| 1862 | 2P | BG4NUC-HC40 | BG4-12 | 5453 | 2P | BG10NUC-HC107 | BG10-12 |
| 1864 | RBD | BG4NUC-HC42 | BG4-13 | 5457 | RBD | BG10NUC-HC111 | BG10-13 |
| 1866 | 2P | BG4NUC-HC44 | BG4-14 | 5459 | 2P | BG10NUC-HC113 | BG10-14 |
| 1868 | 2F | BG4NUC-HC46 | BG4-15 | 5461 | RBD | BG10NUC-HC115 | BG10-15 |
| 1870 | RBD | BG4NUC-HC48 | BG4-16 | 5467 | 2P | BG10NUC-HC121 | BG10-16 |
| 1872 | 2F | BG4NUC-HC50 | BG4-17 | 5471 | 2P | BG10NUC-HC125 | BG10-17 |
| 1874 | 2P | BG4NUC-HC52 | BG4-18 | 5473 | RBD | BG10NUC-HC127 | BG10-18 |
| 1876 | RBD | BG4NUC-HC54 | BG4-19 | 5475 | RBD | BG10NUC-HC129 | BG10-19 |
| 1879 | 2P | BG4NUC-HC57 | BG4-20 | 5774 | RBD | BG10NUC-LC1 | BG10-1 |
| 1880 | RBD | BG4NUC-HC58 | BG4-21 | 5810 | RBD | BG10NUC-LC37 | BG10-2 |
| 1955 | 2P | BG4NUC-HC133 | BG4-22 | 5825 | RBD | BG10NUC-LC52 | BG10-3 |
| 1980 | RBD | BG4NUC-HC158 | BG4-23 | 5834 | RBD | BG10NUC-LC61 | BG10-4 |
| 2039 | RBD | BG4NUC-HC217 | BG4-24 | 5843 | RBD | BG10NUC-LC70 | BG10-5 |
| 2051 | 2P | BG4NUC-HC229 | BG4-25 | 5851 | RBD | BG10NUC-LC78 | BG10-6 |
| 2128 | RBD | BG4NUC-HC306 | BG4-26 | 5858 | RBD | BG10NUC-LC85 | BG10-7 |
| 2205 | 2P | BG4NUC-HC383 | BG4-27 | 5863 | RBD | BG10NUC-LC90 | BG10-8 |
| 2232 | 2P | BG4NUC-LC1 | BG4-1 | 5868 | 2P | BG10NUC-LC95 | BG10-9 |
| 2243 | 2P | BG4NUC-LC12 | BG4-2 | 5873 | RBD | BG10NUC-LC100 | BG10-10 |
| 2247 | 2P | BG4NUC-LC16 | BG4-3 | 5877 | RBD | BG10NUC-LC104 | BG10-11 |
| 2252 | 2P | BG4NUC-LC21 | BG4-4 | 5880 | 2P | BG10NUC-LC107 | BG10-12 |
| 2254 | RBD | BG4NUC-LC23 | BG4-5 | 5884 | RBD | BG10NUC-LC111 | BG10-13 |
| 2256 | RBD | BG4NUC-LC25 | BG4-6 | 5886 | 2P | BG10NUC-LC113 | BG10-14 |
| 2259 | RBD | BG4NUC-LC28 | BG4-7 | 5888 | RBD | BG10NUC-LC115 | BG10-15 |
| 2262 | RBD | BG4NUC-LC31 | BG4-8 | 5894 | 2P | BG10NUC-LC121 | BG10-16 |
| 2265 | RBD | BG4NUC-LC34 | BG4-9 | 5898 | 2P | BG10NUC-LC125 | BG10-17 |
| 2268 | RBD | BG4NUC-LC37 | BG4-10 | 5900 | RBD | BG10NUC-LC127 | BG10-18 |
| 2269 | RBD | BG4NUC-LC38 | BG4-11 | 5902 | RBD | BG10NUC-LC129 | BG10-19 |

### REFERENCES

Adams, P. D., Afonine, P. V., Bunkoczi, G., Chen, V. B., Davis, I. W., Echols, N., Headd, J. J., Hung, L. W., Kapral, G. J., Grosse-Kunstleve, R. W., et al. (2010). PHENIX: a comprehensive Python-based system for macromolecular structure solution. Acta Crystallogr D Biol Crystallogr 66, 213-221.
Barnes, C. O., Jette, C. A., Abernathy, M. E., Dam, K.-M. A., Esswein, S. R., Gristick, H. B., Malyutin, A. G., Sharaf, N. G., Huey-Tubman, K. E., Lee, Y. E., et al. (2020a). SARS-COV-2 neutralizing antibody structures inform therapeutic strategies. Nature.
Barnes, C. O., West, A. P., Jr., Huey-Tubman, K. E., Hoffmann, M. A. G., Sharaf, N. G., Hoffman, P. R., Koranda, N., Gristick, H. B., Gaebler, C., Muecksch, F., et al. (2020b). Structures of Human Antibodies Bound to SARS-COV-2 Spike Reveal Common Epitopes and Recurrent Features of Antibodies. Cell 182, 828-842 e816.
Baum, A., Fulton, B. O., Wloga, E., Copin, R., Pascal, K. E., Russo, V., Giordano, S., Lanza, K., Negron, N., Ni, M., et al. (2020). Antibody cocktail to SARS-COV-2 spike protein prevents rapid mutational escape seen with individual antibodies. Science 369, 1014-1018.
Bournazos, S., Gupta, A., and Ravetch, J. V. (2020). The role of IgG Fc receptors in antibody-dependent enhancement. Nat Rev Immunol 20, 633-643.
Brouwer, P. J. M., Caniels, T. G., van der Straten, K., Snitselaar, J. L., Aldon, Y., Bangaru, S., Torres, J. L., Okba, N. M. A., Claireaux, M., Kerster, G., et al. (2020). Potent neutralizing antibodies from COVID-19 patients define multiple targets of vulnerability. Science 369, 643-650.
Butler, A., Hoffman, P., Smibert, P., Papalexi, E., and Satija, R. (2018). Integrating single-cell transcriptomic data across different conditions, technologies, and species. Nat Biotechnol 36, 411-420.
Cao, Y., Su, B., Guo, X., Sun, W., Deng, Y., Bao, L., Zhu, Q., Zhang, X., Zheng, Y., Geng, C., et al. (2020a). Potent neutralizing antibodies against SARS-COV-2 identified by high-throughput single-cell sequencing of convalescent patients' B cells. Cell.
Cao, Y., Su, B., Guo, X., Sun, W., Deng, Y., Bao, L., Zhu, Q., Zhang, X., Zheng, Y., Geng, C., et al. (2020b). Potent Neutralizing Antibodies against SARS-COV-2 Identified by High-Throughput Single-Cell Sequencing of Convalescent Patients' B Cells. Cell 182, 73-84 e16.
Caskey, M., Klein, F., and Nussenzweig, M. C. (2019). Broadly neutralizing anti-HIV-1 monoclonal antibodies in the clinic. Nat Med 25, 547-553.
Chao, A., Gotelli, N. J., Hsieh, T. C., Sander, E. L., Ma, K. H., Colwell, R. K., and Ellison, A. M. (2014). Rarefaction and extrapolation with Hill numbers: a framework for sampling and estimation in species diversity studies. Ecological Monographs 84, 45-67.
Chao, A., Hsieh, T. C., Chazdon, R. L., Colwell, R. K., and Gotelli, N. J. (2015). Unveiling the species-rank abundance distribution by generalizing the Good-Turing sample coverage theory. Ecology 96, 1189-1201.
Chen, F., Tzarum, N., Wilson, I. A., and Law, M. (2019). VH1-69 antiviral broadly neutralizing antibodies: genetics, structures, and relevance to rational vaccine design. Curr Opin Virol 34, 149-159.
Chen, P., Nirula, A., Heller, B., Gottlieb, R. L., Boscia, J., Morris, J., Huhn, G., Cardona, J., Mocherla, B., Stosor, V., et al. (2020). SARS-COV-2 Neutralizing Antibody LY-CoV555 in Outpatients with Covid-19. N Engl J Med.
Chen, V. B., Arendall, W. B., 3rd, Headd, J. J., Keedy, D. A., Immormino, R. M., Kapral, G. J., Murray, L. W., Richardson, J. S., and Richardson, D. C. (2010). MolProbity: all-atom structure validation for macromolecular crystallography. Acta Crystallogr D Biol Crystallogr 66, 12-21.
Chen, Y. Q., Wohlbold, T. J., Zheng, N. Y., Huang, M., Huang, Y., Neu, K. E., Lee, J., Wan, H., Rojas, K. T., Kirkpatrick, E., et al. (2018). Influenza Infection in Humans Induces Broadly Cross-Reactive and Protective Neuraminidase-Reactive Antibodies. Cell 173, 417-429 e410.
Chi, X., Yan, R., Zhang, J., Zhang, G., Zhang, Y., Hao, M., Zhang, Z., Fan, P., Dong, Y., Yang, Y., et al. (2020). A neutralizing human antibody binds to the N-terminal domain of the Spike protein of SARS-COV-2. Science 369, 650-655.
Cohen, A. A., Gnanapragasam, P. N. P., Lee, Y. E., Hoffman, P. R., Ou, S., Kakutani, L. M., Keeffe, J. R., Barnes, C. O., Nussenzweig, M. C., and Bjorkman, P. J. (2020). Mosaic RBD nanoparticles elicit neutralizing antibodies against SARS-COV-2 and zoonotic coronaviruses. bioRxiv.
Corti, D., and Lanzavecchia, A. (2013). Broadly neutralizing antiviral antibodies. Annu Rev Immunol 31, 705-742.
Corti, D., Zhao, J., Pedotti, M., Simonelli, L., Agnihothram, S., Fett, C., Fernandez-Rodriguez, B., Foglierini, M., Agatic, G., Vanzetta, F., et al. (2015). Prophylactic and postexposure efficacy of a potent human monoclonal antibody against MERS coronavirus. Proc Natl Acad Sci USA 112, 10473-10478.
Crawford, K. H. D., Dingens, A. S., Eguia, R., Wolf, C. R., Wilcox, N., Logue, J. K., Shuey, K., Casto, A. M., Fiala, B., Wrenn, S., et al. (2020a). Dynamics of neutralizing antibody titers in the months after SARS-COV-2 infection. J Infect Dis.
Crawford, K. H. D., Eguia, R., Dingens, A. S., Loes, A. N., Malone, K. D., Wolf, C. R., Chu, H. Y., Tortorici, M. A., Veesler, D., Murphy, M., et al. (2020b). Protocol and Reagents for Pseudotyping Lentiviral Particles with SARS-COV-2 Spike Protein for Neutralization Assays. Viruses 12.
Davies, N. G., Abbott, S., Barnard, R. C., Jarvis, C. I., Kucharski, A. J., Munday, J., Pearson, C. A. B., Russell, T. W., Tully, D. C., Washburne, A. D., et al. (2021). Estimated transmissibility and severity of novel SARS-COV-2 Variant of Concern 202012/01 in England. medRxiv, 2020.2012.2024.20248822.
de Wit, E., van Doremalen, N., Falzarano, D., and Munster, V. J. (2016). SARS and MERS: recent insights into emerging coronaviruses. Nat Rev Microbiol 14, 523-534.
Diskin, R., Klein, F., Horwitz, J. A., Halper-Stromberg, A., Sather, D. N., Marcovecchio, P. M., Lee, T., West, A. P., Jr., Gao, H., Seaman, M. S., et al. (2013). Restricting HIV-1 pathways for escape using rationally designed anti-HIV-1 antibodies. J Exp Med 210, 1235-1249.
Dunbar, J., Krawczyk, K., Leem, J., Baker, T., Fuchs, A., Georges, G., Shi, J., and Deane, C. M. (2014). SAbDab: the structural antibody database. Nucleic Acids Res 42, D1140-1146.
Ellebedy, A. H., Jackson, K. J., Kissick, H. T., Nakaya, H. I., Davis, C. W., Roskin, K. M., McElroy, A. K., Oshansky, C. M., Elbein, R., Thomas, S., et al. (2016). Defining antigen-specific plasmablast and memory B cell subsets in human blood after viral infection or vaccination. Nat Immunol 17, 1226-1234.
Emsley, P., Lohkamp, B., Scott, W. G., and Cowtan, K. (2010). Features and development of Coot. Acta Crystallogr D Biol Crystallogr 66, 486-501.
Goddard, T. D., Huang, C. C., Meng, E. C., Pettersen, E. F., Couch, G. S., Morris, J. H., and Ferrin, T. E. (2018). UCSF ChimeraX: Meeting modern challenges in visualization and analysis. Protein Sci 27, 14-25.
Greaney, A. J., Starr, T. N., Gilchuk, P., Zost, S. J., Binshtein, E., Loes, A. N., Hilton, S. K., Huddleston, J., Eguia, R., Crawford, K. H. D., et al. (2020). Complete Mapping of Mutations to the SARS-COV-2 Spike Receptor-Binding Domain that Escape Antibody Recognition. Cell Host Microbe.
Gristick, H. B., von Boehmer, L., West, A. P., Jr., Schamber, M., Gazumyan, A., Golijanin, J., Seaman, M. S., Fatkenheuer, G., Klein, F., Nussenzweig, M. C., et al. (2016). Natively glycosylated HIV-1 Env structure reveals new mode for antibody recognition of the CD4-binding site. Nat Struct Mol Biol 23, 906-915.
Gupta, N. T., Vander Heiden, J. A., Uduman, M., Gadala-Maria, D., Yaari, G., and Kleinstein, S. H. (2015). Change-O: a toolkit for analyzing large-scale B cell immunoglobulin repertoire sequencing data. Bioinformatics 31, 3356-3358.
Guthmiller, J. J., Lan, L. Y., Fernandez-Quintero, M. L., Han, J., Utset, H. A., Bitar, D. J., Hamel, N. J., Stovicek, O., Li, L., Tepora, M., et al. (2020). Polyreactive Broadly Neutralizing B cells Are Selected to Provide Defense against Pandemic Threat Influenza Viruses. Immunity 53, 1230-1244 e1235.
Hansen, J., Baum, A., Pascal, K. E., Russo, V., Giordano, S., Wloga, E., Fulton, B. O., Yan, Y., Koon, K., Patel, K., et al. (2020). Studies in humanized mice and convalescent humans yield a SARS-COV-2 antibody cocktail. Science.
Hill, M. O. (1973). Diversity and Evenness: A Unifying Notation and Its Consequences. Ecology 54, 427-432.
Horns, F., Dekker, C. L., and Quake, S. R. (2020). Memory B Cell Activation, Broad Anti-influenza Antibodies, and Bystander Activation Revealed by Single-Cell Transcriptomics. Cell Rep 30, 905-913 e906.
Horwitz, J. A., Halper-Stromberg, A., Mouquet, H., Gitlin, A. D., Tretiakova, A., Eisenreich, T. R., Malbec, M., Gravemann, S., Billerbeck, E., Dorner, M., et al. (2013). HIV-1 suppression and durable control by combining single broadly neutralizing antibodies and antiretroviral drugs in humanized mice. Proc Natl Acad Sci USA 110, 16538-16543.
Hotzel, I., Theil, F. P., Bernstein, L. J., Prabhu, S., Deng, R., Quintana, L., Lutman, J., Sibia, R., Chan, P., Bumbaca, D., et al. (2012). A strategy for risk mitigation of antibodies with fast clearance. MAbs 4, 753-760.
Hsieh, C. L., Goldsmith, J. A., Schaub, J. M., DiVenere, A. M., Kuo, H. C., Javanmardi, K., Le, K. C., Wrapp, D., Lee, A. G., Liu, Y., et al. (2020). Structure-based design of prefusion-stabilized SARS-COV-2 spikes. Science 369, 1501-1505.
Hurlburt, N. K., Seydoux, E., Wan, Y. H., Edara, V. V., Stuart, A. B., Feng, J., Suthar, M. S., McGuire, A. T., Stamatatos, L., and Pancera, M. (2020). Structural basis for potent neutralization of SARS-COV-2 and role of antibody affinity maturation. Nat Commun 11, 5413.
Izawa, K., Martin, E., Soudais, C., Bruneau, J., Boutboul, D., Rodriguez, R., Lenoir, C., Hislop, A. D., Besson, C., Touzot, F., et al. (2017). Inherited CD70 deficiency in humans reveals a critical role for the CD70-CD27 pathway in immunity to Epstein-Barr virus infection. J Exp Med 214, 73-89.
Johnson, W. E., Li, C., and Rabinovic, A. (2007). Adjusting batch effects in microarray expression data using empirical Bayes methods. Biostatistics 8, 118-127.
Ju, B., Zhang, Q., Ge, J., Wang, R., Sun, J., Ge, X., Yu, J., Shan, S., Zhou, B., Song, S., et al. (2020). Human neutralizing antibodies elicited by SARS-COV-2 infection. Nature 584, 115-119
Kabsch, W. (2010). XDS. Acta Crystallogr D Biol Crystallogr 66, 125-132.
Klein, J. S., and Bjorkman, P. J. (2010). Few and far between: how HIV may be evading antibody avidity. PLOS Pathog 6, e1000908.
Kremmidiotis, G., and Zola, H. (1995). Changes in CD44 expression during B cell differentiation in the human tonsil. Cell Immunol 161, 147-157.
Krissinel, E., and Henrick, K. (2007). Inference of macromolecular assemblies from crystalline state. Journal of molecular biology 372, 774-797.
Kulkarni, P. S., Hurwitz, J. L., Simoes, E. A. F., and Piedra, P. A. (2018). Establishing Correlates of Protection for Vaccine Development: Considerations for the Respiratory Syncytial Virus Vaccine Field. Viral Immunol 31, 195-203.
Kyte, J., and Doolittle, R. F. (1982). A simple method for displaying the hydropathic character of a protein. Journal of molecular biology 157, 105-132.
Lam, J. H., and Baumgarth, N. (2019). The Multifaceted B Cell Response to Influenza Virus. J Immunol 202, 351-359.
Lefranc, M. P., Giudicelli, V., Duroux, P., Jabado-Michaloud, J., Folch, G., Aouinti, S., Carillon, E., Duvergey, H., Houles, A., Paysan-Lafosse, T., et al. (2015). IMGT(R), the international ImMunoGeneTics information system(R) 25 years on. Nucleic Acids Res 43, D413-422.
Leyendeckers, H., Odendahl, M., Lohndorf, A., Irsch, J., Spangfort, M., Miltenyi, S., Hunzelmann, N., Assenmacher, M., Radbruch, A., and Schmitz, J. (1999). Correlation analysis between frequencies of circulating antigen-specific IgG-bearing memory B cells and serum titers of antigen-specific IgG. Eur J Immunol 29, 1406-1417.
Li, B., Gould, J., Yang, Y., Sarkizova, S., Tabaka, M., Ashenberg, O., Rosen, Y., Slyper, M., Kowalczyk, M. S., Villani, A. C., et al. (2020). Cumulus provides cloud-based data analysis for large-scale single-cell and single-nucleus RNA-seq. Nat Methods 17, 793-798.
Li, W., Moore, M. J., Vasilieva, N., Sui, J., Wong, S. K., Berne, M. A., Somasundaran, M., Sullivan, J. L., Luzuriaga, K., Greenough, T. C., et al. (2003). Angiotensin-converting enzyme 2 is a functional receptor for the SARS coronavirus. Nature 426, 450-454.
Liu, L., Wang, P., Nair, M. S., Yu, J., Rapp, M., Wang, Q., Luo, Y., Chan, J. F., Sahi, V., Figueroa, A., et al. (2020). Potent neutralizing antibodies against multiple epitopes on SARS-COV-2 spike. Nature 584, 450-456.
Liu, Y., Liu, J., Xia, H., Zhang, X., Fontes-Garfias, C. R., Swanson, K. A., Cai, H., Sarkar, R., Chen, W., Cutler, M., et al. (2021). Neutralizing Activity of BNT162b2-Elicited Serum—Preliminary Report. N Engl J Med.
Lv, Z., Deng, Y. Q., Ye, Q., Cao, L., Sun, C. Y., Fan, C., Huang, W., Sun, S., Sun, Y., Zhu, L., et al. (2020). Structural basis for neutralization of SARS-COV-2 and SARS-COV by a potent therapeutic antibody. Science 369, 1505-1509.
Mastronarde, D. N. (2005). Automated electron microscope tomography using robust prediction of specimen movements. J Struct Biol 152, 36-51.
Mathew, N. R., Jayanthan, J. K., Smirnov, I., Robinson, J. L., Axelsson, H., Nakka, S. S., Emmanouilidi, A., Czarnewski, P., Yewdell, W. T., Lebrero-Fernández, C., et al. (2020). Single cell BCR and transcriptome analysis after respiratory virus infection reveals spatiotemporal dynamics of antigen-specific B cell responses. bioRxiv, 2020.2008.2024.264069.
McCoy, A. J., Grosse-Kunstleve, R. W., Adams, P. D., Winn, M. D., Storoni, L. C., and Read, R. J. (2007). Phaser crystallographic software. J Appl Crystallogr 40, 658-674.
Moir, S., Ho, J., Malaspina, A., Wang, W., DiPoto, A. C., O'Shea, M. A., Roby, G., Kottilil, S., Arthos, J., Proschan, M. A., et al. (2008). Evidence for HIV-associated B cell exhaustion in a dysfunctional memory B cell compartment in HIV-infected viremic individuals. J Exp Med 205, 1797-1805.
Moore, D. S. (1985). Amino acid and peptide net charges: A simple calculational procedure. Biochemical Education 13, 10-11.
Moroney, J. B., Vasudev, A., Pertsemlidis, A., Zan, H., and Casali, P. (2020). Integrative transcriptome and chromatin landscape analysis reveals distinct epigenetic regulations in human memory B cells. Nat Commun 11, 5435.
Mouquet, H., Scheid, J. F., Zoller, M. J., Krogsgaard, M., Ott, R. G., Shukair, S., Artyomov, M. N., Pietzsch, J., Connors, M., Pereyra, F., et al. (2010). Polyreactivity increases the apparent affinity of anti-HIV antibodies by heteroligation. Nature 467, 591-595.
Muehlinghaus, G., Cigliano, L., Huehn, S., Peddinghaus, A., Leyendeckers, H., Hauser, A. E., Hiepe, F., Radbruch, A., Arce, S., and Manz, R. A. (2005). Regulation of CXCR3 and CXCR4 expression during terminal differentiation of memory B cells into plasma cells. Blood 105, 3965-3971.
Mulangu, S., Dodd, L. E., Davey, R. T., Jr., Tshiani Mbaya, O., Proschan, M., Mukadi, D., Lusakibanza Manzo, M., Nzolo, D., Tshomba Oloma, A., Ibanda, A., et al. (2019). A Randomized, Controlled Trial of Ebola Virus Disease Therapeutics. N Engl J Med 381, 2293-2303.
Nagasawa, T., Nakajima, T., Tachibana, K., Iizasa, H., Bleul, C. C., Yoshie, O., Matsushima, K., Yoshida, N., Springer, T. A., and Kishimoto, T. (1996). Molecular cloning and characterization of a murine pre-B-cell growth-stimulating factor/stromal cell-derived factor 1 receptor, a murine homolog of the human immunodeficiency virus 1 entry coreceptor fusin. Proc Natl Acad Sci USA 93, 14726-14729.
Neu, K. E., Guthmiller, J. J., Huang, M., La, J., Vieira, M. C., Kim, K., Zheng, N. Y., Cortese, M., Tepora, M. E., Hamel, N. J., et al. (2019). Spec-seq unveils transcriptional subpopulations of antibody-secreting cells following influenza vaccination. J Clin Invest 129, 93-105.
Nie, Y., Waite, J., Brewer, F., Sunshine, M. J., Littman, D. R., and Zou, Y. R. (2004). The role of CXCR4 in maintaining peripheral B cell compartments and humoral immunity. J Exp Med 200, 1145-1156.
Nouri, N., and Kleinstein, S. H. (2018). A spectral clustering-based method for identifying clones from high-throughput B cell repertoire sequencing data. Bioinformatics 34, 1341-1349.
Piccoli, L., Park, Y.-J., Tortorici, M. A., Czudnochowski, N., Walls, A. C., Beltramello, M., Silacci-Fregni, C., Pinto, D., Rosen, L. E., Bowen, J. E., et al. (2020). Mapping neutralizing and immunodominant sites on the SARS-COV-2 spike receptor-binding domain by structure-guided high-resolution serology. Cell.
Pinto, D., Park, Y.-J., Beltramello, M., Walls, A. C., Tortorici, M. A., Bianchi, S., Jaconi, S., Culap, K., Zatta, F., De Marco, A., et al. (2020a). Cross-neutralization of SARS-COV-2 by a human monoclonal SARS-COV antibody. Nature 583, 290-295.
Pinto, D., Park, Y. J., Beltramello, M., Walls, A. C., Tortorici, M. A., Bianchi, S., Jaconi, S., Culap, K., Zatta, F., De Marco, A., et al. (2020b). Cross-neutralization of SARS-COV-2 by a human monoclonal SARS-COV antibody. Nature 583, 290-295.
Prabakaran, P., Gan, J., Feng, Y., Zhu, Z., Choudhry, V., Xiao, X., Ji, X., and Dimitrov, D. S. (2006). Structure of severe acute respiratory syndrome coronavirus receptor-binding domain complexed with neutralizing antibody. J Biol Chem 281, 15829-15836.
Prigent, J., Jarossay, A., Planchais, C., Eden, C., Dufloo, J., Kok, A., Lorin, V., Vratskikh, O., Couderc, T., Bruel, T., et al. (2018). Conformational Plasticity in Broadly Neutralizing HIV-1 Antibodies Triggers Polyreactivity. Cell Rep 23, 2568-2581.
Punjani, A., Rubinstein, J. L., Fleet, D. J., and Brubaker, M. A. (2017). cryoSPARC: algorithms for rapid unsupervised cryo-EM structure determination. Nat Methods 14, 290-296.
Robbiani, D. F., Bozzacco, L., Keeffe, J. R., Khouri, R., Olsen, P. C., Gazumyan, A., Schaefer-Babajew, D., Avila-Rios, S., Nogueira, L., Patel, R., et al. (2017). Recurrent Potent Human Neutralizing Antibodies to Zika Virus in Brazil and Mexico. Cell 169, 597-609 e511.
Robbiani, D. F., Gaebler, C., Muecksch, F., Lorenzi, J. C. C., Wang, Z., Cho, A., Agudelo, M., Barnes, C. O., Gazumyan, A., Finkin, S., et al. (2020). Convergent antibody responses to SARS-COV-2 in convalescent individuals. Nature 584, 437-442.
Rockx, B., Corti, D., Donaldson, E., Sheahan, T., Stadler, K., Lanzavecchia, A., and Baric, R. (2008). Structural basis for potent cross-neutralizing human monoclonal antibody protection against lethal human and zoonotic severe acute respiratory syndrome coronavirus challenge. J Virol 82, 3220-3235.
Rogers, T. F., Zhao, F., Huang, D., Beutler, N., Burns, A., He, W. T., Limbo, O., Smith, C., Song, G., Woehl, J., et al. (2020). Isolation of potent SARS-COV-2 neutralizing antibodies and protection from disease in a small animal model. Science 369, 956-963.
Roy, V., Fischinger, S., Atyeo, C., Slein, M., Loos, C., Balazs, A., Luedemann, C., Astudillo, M. G., Yang, D., Wesemann, D. R., et al. (2020). SARS-COV-2-specific ELISA development. J Immunol Methods 484-485, 112832.
Rubelt, F., Sievert, V., Knaust, F., Diener, C., Lim, T. S., Skriner, K., Klipp, E., Reinhardt, R., Lehrach, H., and Konthur, Z. (2012). Onset of immune senescence defined by unbiased pyrosequencing of human immunoglobulin mRNA repertoires. PLOS One 7, e49774.
Scheid, J. F., Horwitz, J. A., Bar-On, Y., Kreider, E. F., Lu, C. L., Lorenzi, J. C., Feldmann, A., Braunschweig, M., Nogueira, L., Oliveira, T., et al. (2016). HIV-1 antibody 3BNC117 suppresses viral rebound in humans during treatment interruption. Nature 535, 556-560.
Scheid, J. F., Mouquet, H., Feldhahn, N., Seaman, M. S., Velinzon, K., Pietzsch, J., Ott, R. G., Anthony, R. M., Zebroski, H., Hurley, A., et al. (2009a). Broad diversity of neutralizing antibodies isolated from memory B cells in HIV-infected individuals. Nature 458, 636-640.
Scheid, J. F., Mouquet, H., Feldhahn, N., Walker, B. D., Pereyra, F., Cutrell, E., Seaman, M. S., Mascola, J. R., Wyatt, R. T., Wardemann, H., et al. (2009b). A method for identification of HIV gp140 binding memory B cells in human blood. J Immunol Methods 343, 65-67.
Scheid, J. F., Mouquet, H., Ueberheide, B., Diskin, R., Klein, F., Oliveira, T. Y., Pietzsch, J., Fenyo, D., Abadir, A., Velinzon, K., et al. (2011). Sequence and structural convergence of broad and potent HIV antibodies that mimic CD4 binding. Science 333, 1633-1637.
Schmidt, F., Weisblum, Y., Muecksch, F., Hoffmann, H. H., Michailidis, E., Lorenzi, J. C. C., Mendoza, P., Rutkowska, M., Bednarski, E., Gaebler, C., et al. (2020). Measuring SARS-CoV-2 neutralizing antibody activity using pseudotyped and chimeric viruses. J Exp Med 217.
Shi, R., Shan, C., Duan, X., Chen, Z., Liu, P., Song, J., Song, T., Bi, X., Han, C., Wu, L., et al. (2020). A human neutralizing antibody targets the receptor-binding site of SARS-COV-2. Nature 584, 120-124.
Shingai, M., Donau, O. K., Plishka, R. J., Buckler-White, A., Mascola, J. R., Nabel, G. J., Nason, M. C., Montefiori, D., Moldt, B., Poignard, P., et al. (2014). Passive transfer of modest titers of potent and broadly neutralizing anti-HIV monoclonal antibodies block SHIV infection in macaques. J Exp Med 211, 2061-2074.
Stuart, T., Butler, A., Hoffman, P., Hafemeister, C., Papalexi, E., Mauck, W. M., 3rd, Hao, Y., Stoeckius, M., Smibert, P., and Satija, R. (2019). Comprehensive Integration of Single-Cell Data. Cell 177, 1888-1902 e1821.
Sui, J., Li, W., Murakami, A., Tamin, A., Matthews, L. J., Wong, S. K., Moore, M. J., Tallarico, A. S., Olurinde, M., Choe, H., et al. (2004). Potent neutralization of severe acute respiratory syndrome (SARS) coronavirus by a human mAb to S1 protein that blocks receptor association. Proc Natl Acad Sci USA 101, 2536-2541.
Tada, T., Fan, C., Chen, J. S., Kaur, R., Stapleford, K. A., Gristick, H., Dcosta, B. M., Wilen, C. B., Nimigean, C. M., and Landau, N. R. (2020). An ACE2 Microbody Containing a Single Immunoglobulin Fc Domain Is a Potent Inhibitor of SARS-COV-2. Cell Rep 33, 108528.
Tan, T. J. C., Yuan, M., Kuzelka, K., Padron, G. C., Beal, J. R., Chen, X., Wang, Y., Rivera-Cardona, J., Zhu, X., Stadtmueller, B. M., et al. (2021). Sequence signatures of two IGHV3-53/3-66 public clonotypes to SARS-COV-2 receptor binding domain. bioRxiv.
Tang, X. C., Agnihothram, S. S., Jiao, Y., Stanhope, J., Graham, R. L., Peterson, E. C., Avnir, Y., Tallarico, A. S., Sheehan, J., Zhu, Q., et al. (2014). Identification of human neutralizing antibodies against MERS-COV and their role in virus adaptive evolution. Proc Natl Acad Sci USA III, E2018-2026.
Tegally, H., Wilkinson, E., Giovanetti, M., Iranzadeh, A., Fonseca, V., Giandhari, J., Doolabh, D., Pillay, S., San, E. J., Msomi, N., et al. (2020). Emergence and rapid spread of a new severe acute respiratory syndrome-related coronavirus 2 (SARS-COV-2) lineage with multiple spike mutations in South Africa. medRxiv, 2020.2012.2021.20248640.
Terwilliger, T. C., Adams, P. D., Afonine, P. V., and Sobolev, O. V. (2018). A fully automatic method yielding initial models from high-resolution cryo-electron microscopy maps. Nat Methods 15, 905-908.
Tian, X., Li, C., Huang, A., Xia, S., Lu, S., Shi, Z., Lu, L., Jiang, S., Yang, Z., Wu, Y., et al. (2020). Potent binding of 2019 novel coronavirus spike protein by a SARS coronavirus-specific human monoclonal antibody. Emerg Microbes Infect 9, 382-385.
Tiller, T., Meffre, E., Yurasov, S., Tsuiji, M., Nussenzweig, M. C., and Wardemann, H. (2008). Efficient generation of monoclonal antibodies from single human B cells by single cell RT-PCR and expression vector cloning. J Immunol Methods 329, 112-124.
Tiller, T., Tsuiji, M., Yurasov, S., Velinzon, K., Nussenzweig, M. C., and Wardemann, H. (2007). Autoreactivity in human IgG+ memory B cells. Immunity 26, 205-213.
Tirosh, I., Izar, B., Prakadan, S. M., Wadsworth, M. H., 2^{nd}, Treacy, D., Trombetta, J. J., Rotem, A., Rodman, C., Lian, C., Murphy, G., et al. (2016). Dissecting the multicellular ecosystem of metastatic melanoma by single-cell RNA-seq. Science 352, 189-196.
Tortorici, M. A. (2020). Ultrapotent human antibodies protect against SARS-COV-2 challenge via multiple mechanisms.
Traag, V. A., Waltman, L., and van Eck, N. J. (2019). From Louvain to Leiden: guaranteeing well-connected communities. Sci Rep 9, 5233.
Traggiai, E., Becker, S., Subbarao, K., Kolesnikova, L., Uematsu, Y., Gismondo, M. R., Murphy, B. R., Rappuoli, R., and Lanzavecchia, A. (2004). An efficient method to make human monoclonal antibodies from memory B cells: potent neutralization of SARS coronavirus. Nat Med 10, 871-875.
Triller, G., Scally, S. W., Costa, G., Pissarev, M., Kreschel, C., Bosch, A., Marois, E., Sack, B. K., Murugan, R., Salman, A. M., et al. (2017). Natural Parasite Exposure Induces Protective Human Anti-Malarial Antibodies. Immunity 47, 1197-1209 e1110.
van Gisbergen, K. P., Klarenbeek, P. L., Kragten, N. A., Unger, P. P., Nieuwenhuis, M. B., Wensveen, F. M., ten Brinke, A., Tak, P. P., Eldering, E., Nolte, M. A., et al. (2011). The costimulatory molecule CD27 maintains clonally diverse CD8(+) T cell responses of low antigen affinity to protect against viral variants. Immunity 35, 97-108.
Verschoor, C. P., Singh, P., Russell, M. L., Bowdish, D. M., Brewer, A., Cyr, L., Ward, B. J., and Loeb, M. (2015). Microneutralization assay titres correlate with protection against seasonal influenza H1N1 and H3N2 in children. PLOS One 10, e0131531.
Victora, G. D., and Nussenzweig, M. C. (2012). Germinal centers. Annu Rev Immunol 30, 429-457.
Waickman, A. T., Gromowski, G. D., Rutvisuttinunt, W., Li, T., Siegfried, H., Victor, K., Kuklis, C., Gomootsukavadee, M., McCracken, M. K., Gabriel, B., et al. (2020). Transcriptional and clonal characterization of B cell plasmablast diversity following primary and secondary natural DENV infection. EBioMedicine 54, 102733.
Wan, J., Xing, S., Ding, L., Wang, Y., Gu, C., Wu, Y., Rong, B., Li, C., Wang, S., Chen, K., et al. (2020). Human-IgG-Neutralizing Monoclonal Antibodies Block the SARS-COV-2 Infection. Cell Rep 32, 107918.
Wang, Q., Michailidis, E., Yu, Y., Wang, Z., Hurley, A. M., Oren, D. A., Mayer, C. T., Gazumyan, A., Liu, Z., Zhou, Y., et al. (2020a). A Combination of Human Broadly Neutralizing Antibodies against Hepatitis B Virus HBsAg with Distinct Epitopes Suppresses Escape Mutations. Cell Host Microbe 28, 335-349 e336.
Wang, Z., Lorenzi, J. C. C., Muecksch, F., Finkin, S., Viant, C., Gaebler, C., Cipolla, M., Hoffman, H. H., Oliveira, T. Y., Oren, D. A., et al. (2020b). Enhanced SARS-COV-2 Neutralization by Secretory IgA in vitro. bioRxiv.
Wang, Z., Schmidt, F., Weisblum, Y., Muecksch, F., Barnes, C. O., Finkin, S., Schaefer-Babajew, D., Cipolla, M., Gaebler, C., Lieberman, J. A., et al. (2021). mRNA vaccine-elicited antibodies to SARS-COV-2 and circulating variants. Nature.
Wardemann, H., Yurasov, S., Schaefer, A., Young, J. W., Meffre, E., and Nussenzweig, M. C. (2003). Predominant autoantibody production by early human B cell precursors. Science 301, 1374-1377.
Weisblum, Y., Schmidt, F., Zhang, F., DaSilva, J., Poston, D., Lorenzi, J. C., Muecksch, F., Rutkowska, M., Hoffmann, H. H., Michailidis, E., et al. (2020). Escape from neutralizing antibodies by SARS-COV-2 spike protein variants. Elife 9.
West, A. P., Jr., Galimidi, R. P., Foglesong, C. P., Gnanapragasam, P. N., Klein, J. S., and Bjorkman, P. J. (2010). Evaluation of CD4-CD4i antibody architectures yields potent, broadly cross-reactive anti-human immunodeficiency virus reagents. J Virol 84, 261-269.
West, A. P., Jr., Scharf, L., Horwitz, J., Klein, F., Nussenzweig, M. C., and Bjorkman, P. J. (2013). Computational analysis of anti-HIV-1 antibody neutralization panel data to identify potential functional epitope residues. Proc Natl Acad Sci USA 110, 10598-10603.
Wibmer, C. K., Ayres, F., Hermanus, T., Madzivhandila, M., Kgagudi, P., Lambson, B. E., Vermeulen, M., van den Berg, K., Rossouw, T., Boswell, M., et al. (2021). SARS-COV-2 501Y. V2 escapes neutralization by South African COVID-19 donor plasma. bioRxiv.
Winn, M. D., Ballard, C. C., Cowtan, K. D., Dodson, E. J., Emsley, P., Evans, P. R., Keegan, R. M., Krissinel, E. B., Leslie, A. G., McCoy, A., et al. (2011). Overview of the CCP4 suite and current developments. Acta Crystallogr D Biol Crystallogr 67, 235-242.
Wrammert, J., Smith, K., Miller, J., Langley, W. A., Kokko, K., Larsen, C., Zheng, N. Y., Mays, I., Garman, L., Helms, C., et al. (2008). Rapid cloning of high-affinity human monoclonal antibodies against influenza virus. Nature 453, 667-671.
Wrapp, D., Wang, N., Corbett, K. S., Goldsmith, J. A., Hsieh, C. L., Abiona, O., Graham, B. S., and Mclellan, J. S. (2020). Cryo-EM structure of the 2019-nCOV spike in the prefusion conformation. Science 367, 1260-1263.
Wu, K., Werner, A. P., Koch, M., Choi, A., Narayanan, E., Stewart-Jones, G. B. E., Colpitts, T., Bennett, H., Boyoglu-Barnum, S., Shi, W., et al. (2021). Serum Neutralizing Activity Elicited by mRNA-1273 Vaccine-Preliminary Report. N Engl J Med.
Wu, N. C., Yuan, M., Liu, H., Lee, C. D., Zhu, X., Bangaru, S., Torres, J. L., Caniels, T. G., Brouwer, P. J. M., van Gils, M. J., et al. (2020a). An Alternative Binding Mode of IGHV3-53 Antibodies to the SARS-COV-2 Receptor Binding Domain. Cell Rep 33, 108274.
Wu, Y., Wang, F., Shen, C., Peng, W., Li, D., Zhao, C., Li, Z., Li, S., Bi, Y., Yang, Y., et al. (2020b). A noncompeting pair of human neutralizing antibodies block COVID-19 virus binding to its receptor ACE2. Science.
Yaari, G., Uduman, M., and Kleinstein, S. H. (2012). Quantifying selection in high-throughput Immunoglobulin sequencing data sets. Nucleic Acids Res 40, e134.
Yan, R., Zhang, Y., Li, Y., Xia, L., Guo, Y., and Zhou, Q. (2020). Structural basis for the recognition of the SARS-COV-2 by full-length human ACE2. Science.
Yuan, M., Liu, H., Wu, N. C., Lee, C.-C. D., Zhu, X., Zhao, F., Huang, D., Yu, W., Hua, Y., Tien, H., et al. (2020). Structural basis of a shared antibody response to SARS-COV-2. Science, eabd2321.
Zost, S. J., Gilchuk, P., Case, J. B., Binshtein, E., Chen, R. E., Nkolola, J. P., Schafer, A., Reidy, J. X., Trivette, A., Nargi, R. S., et al. (2020). Potently neutralizing and protective human antibodies against SARS-COV-2. Nature 584, 443-449.
Zuccarino-Catania, G. V., Sadanand, S., Weisel, F. J., Tomayko, M. M., Meng, H., Kleinstein, S. H., Good-Jacobson, K. L., and Shlomchik, M. J. (2014). CD80 and PD-L2 define functionally distinct memory B cell subsets that are independent of antibody isotype. Nat Immunol 15, 631-637.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. An antibody or antigen binding fragment thereof that binds to the SARS-COV-2 receptor-binding domain (RBD) and/or SARS-COV-2 spike trimer comprising:
a complementarity-determining region 3 (CDR3) heavy chain and light chain pair that shares at least 90% identity with the heavy chain and/or light chain of a pair selected from the group consisting of: BG1-1, BG1-3 to BG1-28, BG4-1 to BG4-27, BG7-1 to BG7-7, BG7-9 to BG7-20, and BG10-1 to BG10-19 (SEQ ID NOS: 1-184); or
a heavy chain and light chain pair that shares at least 90% identity with the heavy chain and/or light chain of a pair selected from the group consisting of: BG1-1, BG1-3 to BG1-28, BG4-1 to BG4-27, BG7-1 to BG7-7, BG7-9 to BG7-20, and BG10-1 to BG10-19 (SEQ ID NOS: 185-368).

2. The antibody or antigen binding fragment thereof of claim 1, wherein said antibody or antigen binding fragment is an IgG antibody or antigen binding fragment.

3. The antibody or antigen binding fragment thereof of claim 1, wherein said antibody or antigen binding fragment is a monoclonal antibody.

4. The antibody or antigen binding fragment thereof of claim 1, wherein said CDR3 heavy chain and light chain pair or heavy chain and light chain pair is selected from the group consisting of: BG1-6, BG1-12, BG1-14, BG1-17, BG1-22, BG1-23, BG1-24, BG1-25, BG1-26, BG1-28, BG4-10, BG4-11, BG4-14, BG4-16, BG4-17, BG4-24, BG4-25, BG4-26, BG7-14, BG7-15, BG7-16, BG7-18, BG7-19, BG7-20, BG10-10, BG1014, and BG10-19.

5. The antibody or antigen binding fragment thereof of claim 1, wherein said CDR3 heavy chain and light chain pair or heavy chain and light chain pair is selected from the group consisting of: BG1-22, BG1-24, BG4-25, BG7-15, BG7-20, and BG10-19.

6. The antibody or antigen binding fragment thereof of claim 1, wherein said heavy chain and light chain pair is BG7-15.

7. The antibody or antigen binding fragment thereof of claim 6, wherein said antibody or antigen binding fragment thereof binds the two “down”/one “up” RBD conformation on the SARS COV2 spike trimer with no glycan or interprotomer contacts.

8. The antibody or antigen binding fragment thereof of claim 6, wherein said antibody or antigen binding fragment recognizes an epitope in proximity to RBD residues 439-450.

9. The antibody or antigen binding fragment thereof of claim 6, wherein said antibody or antigen binding fragment blocks RBD-ACE2 interactions.

10. The antibody or antigen binding fragment thereof of claim 1, wherein said heavy chain and light chain pair is BG7-20.

11. The antibody or antigen binding fragment thereof of claim 10, wherein said antibody or antigen binding fragment thereof binds a 2d/1u or 1d/2u binding state in the SARS CoV2 spike trimer.

12. The antibody or antigen binding fragment thereof of claim 1, wherein said heavy chain and light chain pair is BG10-19.

13. The antibody or antigen binding fragment thereof of claim 12, wherein said antibody or antigen binding fragment thereof binds to an all down RBD conformation in the SARS CoV2 spike trimer.

14. The antibody or antigen binding fragment thereof of claim 12, wherein said antibody or antigen binding fragment recognizes an epitope in proximity to the N343-glycan.

15. The antibody or antigen binding fragment thereof of claim 12, wherein said antibody or antigen binding fragment light chain makes secondary contacts with an adjacent RBD.

16. The antibody or antigen binding fragment thereof of claim 12, wherein said antibody or antigen binding fragment heavy chain makes contacts with an epitope in proximity to the N343-glycan and the light chain contacts adjacent down RBDs and interacts with residues that overlap with the ACE2 receptor binding motif.

17. The antibody or antigen binding fragment thereof of claim 1, wherein said heavy chain and light chain pair is BG1-24.

18. The antibody or antigen binding fragment thereof of claim 17, wherein said antibody or antigen binding fragment thereof binds both the up/down RBD conformation on the SARS COV2 spike trimer.

19. The antibody or antigen binding fragment thereof of claim 1, wherein said heavy chain and light chain pair is BG1-22.

20. The antibody or antigen binding fragment thereof of claim 19, wherein said antibody or antigen binding fragment thereof binds only the up RBD conformation on the SARS CoV2 spike trimer.

21. The antibody or antigen binding fragment thereof of claim 1, wherein said antibody or antigen binding fragment is a VH3-53/3-66 antibody.

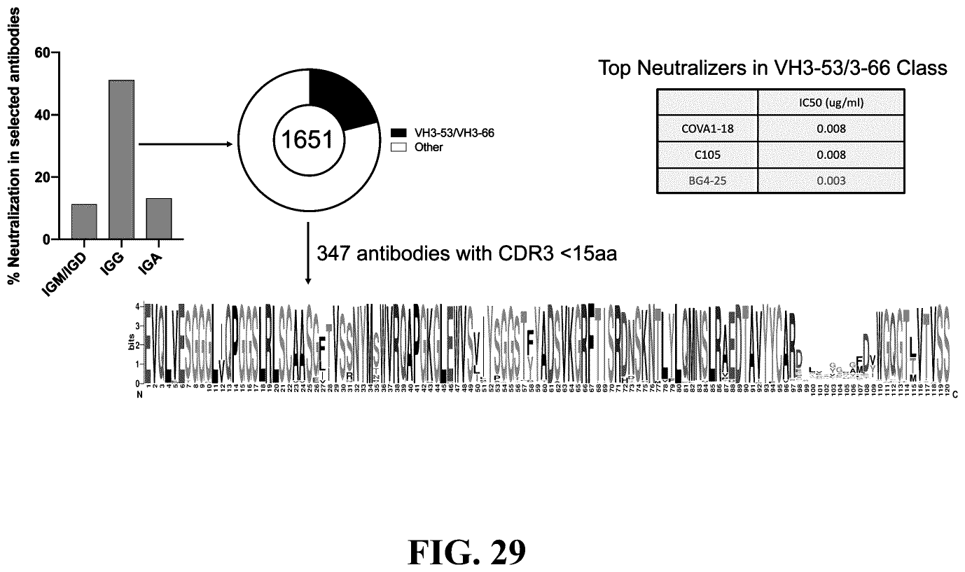

22. The antibody or antigen binding fragment thereof of claim 21, wherein said antibody or antigen binding fragment comprises a CDR3 having a consensus sequence according to FIG. 29.

23. The antibody or antigen binding fragment thereof of claim 21, wherein said heavy chain and light chain pair is BG4-25.

24. An antibody or antigen binding fragment thereof that binds to the SARS, MERS and CoV2 spike trimers outside of the receptor-binding domain (RBD) comprising a complementarity-determining region 3 (CDR3) heavy chain and light chain pair that shares at least 90% identity with the heavy chain and/or light chain of a pair selected from the group consisting of LKA-1-17 (Table 7).

25. The antibody or antigen binding fragment of claim 1, wherein the antibody or antigen binding fragment is modified to enhance stabilization, in vivo half-life, neutralizing activity and/or dimerization.

26. The antibody or antigen binding fragment of claim 25, wherein the antibody or antigen binding fragment is a fusion protein.

27. The antibody or antigen binding fragment of claim 26, wherein the antibody or antigen binding fragment is fused to another antibody or antibody fragment, Fc domain, antigen binding domain, glutathione S-transferase (GST), and/or serum albumin.

28. The antibody or antigen binding fragment of claim 25, wherein the antibody or antigen binding fragment comprises amino acid substitutions that increase antibody binding and/or viral neutralization activity.

29. A method of treating a coronavirus infection comprising administering to a subject in need thereof one or more antibodies or antigen binding fragments of claim 1.

30. The method of claim 29, wherein the coronavirus is selected from the group consisting of SARS-COV-2, SARS and MERS.

31. The method of claim 30, wherein the SARS-COV-2 is a SARS-COV-2 variant.

32. The method of claim 31, wherein the SARS-COV-2 variant is selected from the group consisting of B.1.1.7 and B.1.351.

33. The method of claim 29, wherein BG10-19 is administered.

34. The method of claim 29, wherein BG10-19 and BG4-25 are administered.

35. A method of identifying neutralizing antibodies from a subject infected with a virus comprising selecting antibodies expressed in B cells further expressing a transcriptional program, wherein the transcriptional program comprises one or more genes selected from the group consisting of:
a. GRAMD1C, TMEM156, PDE4D, NFKBIA, S100A10, TKT, CAPG, CXCR3, CFLAR, HMGA1, MARCKSL1, PIM3, RHOF, MIF, ZFP36L1, NME2, MGAT4A, COCH, HOPX, ITGB2-AS1, BASP1, CD80, PAPSS1, CD70, LYPLAL1, LMNA, and FAS; or
b. TKT, ARHGDIB, CFL1, CNN2, S100A10, HSPA8, HMGA1, PPIA, RAC2, CLIC1, SLC25A5, ARPC1B, SELL, PPP1CA, CAPZB, PPP1R18, CAPG, LDHB, S100A4, VIM, LTB, ANXA2, LCP1, TUBB, ACTG1, GAPDH, ACTB, PFN1, CORO1A, and TMSB10; or
c. CD27, CD80, CD46 and CD86.
